(19) United States
(12) Patent Application Publication
(10) Pub. No.: US 2022/0242957 A1
(43) Pub. Date: Aug. 4, 2022

(54) CSF1R/CCR2 MULTISPECIFIC ANTIBODIES

(71) Applicant: MARENGO THERAPEUTICS, INC., Cambridge, MA (US)

(72) Inventors: Andreas LOEW, Cambridge, MA (US); Brian Edward VASH, Cambridge, MA (US); Stephanie J. MAIOCCO, Cambridge, MA (US); Peter MAREK, Cambridge, MA (US)

(21) Appl. No.: 17/280,766

(22) PCT Filed: Sep. 27, 2019
(86) PCT No.: PCT/US2019/053544

§ 371 Date: Mar. 26, 2021

Related U.S. Application Data
(60) Provisional application No. 62/737,742, filed on Sep. 27, 2018.
(60) Provisional application No. 62/750,163, filed on Oct. 24, 2018.
(60) Provisional application No. 62/776,820, filed on Dec. 7, 2018.

(51) Int. Cl. C07K 16/28; C07K 16/46; A61P 35/00

(52) U.S. Cl. CPC C07K 16/2866; C07K 16/2893; C07K 16/2896; A61K 2039/507; C07K 16/2827; A61P 35/00; C07K 16/468

(57) ABSTRACT

Molecules, e.g., multispecific molecules, targeting CSF1R or CCR2 and methods of using the same, are disclosed.

### RELATED APPLICATIONS

This application is a U.S. National Phase Application under 35 U.S.C. § 371 of International Application No. PCT/US2019/053544 filed Sep. 27, 2019, which claims priority to U.S. Provisional Application 62/737,742 filed on Sep. 27, 2018, U.S. Provisional Application 62/750,163 filed on Oct. 24, 2018, and U.S. Provisional Application 62/776,820 filed on Dec. 7, 2018, the entire contents of each of which are hereby incorporated by reference.

### SEQUENCE LISTING

The instant application contains a Sequence Listing which has been submitted electronically in ASCII format and is hereby incorporated by reference in its entirety. Said ASCII copy, created on Sep. 27, 2019, is named 53676_728_601_Seq_Listing.txt and is 711,029 bytes in size.

### BACKGROUND

Multispecific molecules targeting tumor associated macrophages (TAMs) or myeloid derived suppressor cells (MDSCs) and methods of using the same, are disclosed.

### SUMMARY OF THE INVENTION

The disclosure relates, inter alia, to novel multispecific molecules comprising: (i) a first immunosuppressive myeloid cell (IMC) binding moiety (e.g., a first tumor associated macrophage (TAM) binding moiety; or a first myeloid derived suppressor cell (MDSC) binding moiety) (e.g., an antibody molecule); and (ii) a second IMC binding moiety (e.g., a first TAM binding moiety; or a second MDSC binding moiety) (e.g., an antibody molecule), wherein the first and the second IMC (e.g., TAM or MDSC) binding moieties are different. Without being bound by theory, the multispecific molecules disclosed herein are expected to deplete TAMs and/or MDSCs. Accordingly, provided herein are, inter alia, multispecific molecules (e.g., multispecific antibody molecules) that include the aforesaid moieties, nucleic acids encoding the same, methods of producing the aforesaid molecules, and methods of treating a cancer using the aforesaid molecules.

In one aspect, provided herein are isolated multispecific, e.g., a bispecific, molecules, comprising: (i) a first immunosuppressive myeloid cell (IMC) binding moiety (e.g., a first tumor associated macrophage (TAM) binding moiety; or a first myeloid derived suppressor cell (MDSC) binding moiety) (e.g., an antibody molecule); and (ii) a second IMC binding moiety (e.g., a second TAM binding moiety; or a second MDSC binding moiety) (e.g., an antibody molecule), wherein the first and the second IMC (e.g., TAM or MDSC) binding moieties are different. In some embodiments, the first and the second IMC (e.g., TAM or MDSC) binding moieties bind to different epitopes. In some embodiments, the first and the second IMC (e.g., TAM or MDSC) binding moieties bind to different antigens.

In some embodiments, the first IMC binding moiety is a first MDSC binding moiety; and the second IMC binding moiety is a second MDSC binding moiety. In some embodiments, the first IMC binding moiety is a first TAM binding moiety; and the second IMC binding moiety is a second TAM binding moiety. In some embodiments, the first TAM binding moiety binds to CSF1R, CCR2, CXCR2, CD86, CD163, CX3CR1, MARCO, CD204, CD52, folate receptor beta, or PD-L1; and the second TAM binding moiety binds to CCR2, CSF1R, CXCR2, CD86, CD163, CX3CR1, MARCO, CD204, CD52, folate receptor beta, or PD-L1. In some embodiments, the first TAM binding moiety binds to CSF1R, CCR2, CXCR2, or PD-L1 (e.g., human CSF1R, CCR2, CXCR2, or PD-L1) and the second TAM binding moiety binds to CCR2, CSF1R, CXCR2, or PD-L1 (e.g., human CCR2, CSF1R, CXCR2, or PD-L1). In some embodiments, the first TAM binding moiety binds to CSF1R and the second TAM binding moiety binds to CCR2. In some embodiments, the first TAM binding moiety binds to CSF1R and the second TAM binding moiety binds to CXCR2. In some embodiments, the first TAM binding moiety binds to CCR2 and the second TAM binding moiety binds to CXCR2. In some embodiments, the first TAM binding moiety binds to CSF1R and the second TAM binding moiety binds to PD-L1. In some embodiments, the first TAM binding moiety binds to CCR2 and the second TAM binding moiety binds to PD-L1. In some embodiments, the first TAM binding moiety binds to CXCR2 and the second TAM binding moiety binds to PD-L1.

In some embodiments, the first TAM binding moiety binds to CSF1R, CCR2, CXCR2, or PD-L1 with a dissociation constant of less than about 10 nM, and more typically, 10-100 pM; and the second TAM binding moiety binds to CCR2, CSF1R, CXCR2, or PD-L1 with a dissociation constant of less than about 10 nM, and more typically, 10-100 pM. In some embodiments, the first TAM binding moiety binds to a conformational or a linear epitope on CSF1R, CCR2, CXCR2, or PD-L1; and the second TAM binding moiety binds to a conformational or a linear epitope on CCR2, CSF1R, CXCR2, or PD-L1.

In some embodiments, the multispecific molecule comprises at least two non-contiguous polypeptide chains. In some embodiments, the first IMC binding moiety comprises a first anti-IC antibody molecule and/or the second IMC binding moiety comprises a second anti-IMC antibody molecule. In some embodiments, the first anti-IMC antibody molecule and the second anti-IMC antibody molecule are, independently, a full antibody (e.g., an antibody that includes at least one, and preferably two, complete heavy chains, and at least one, and preferably two, complete light chains), or an antigen-binding fragment (e.g., a Fab, F(ab′)2, Fv, a scFv, a single domain antibody, or a diabody (dAb)).

In some embodiments, the first anti-IMC antibody molecule and/or the second anti-IMC antibody molecule comprises a heavy chain constant region chosen from IgG1, IgG2, IgG3, or IgG4, or a fragment thereof.

In some embodiments, the first anti-IMC antibody molecule and/or the second anti-IMC antibody molecule comprises a light chain constant region chosen from the light chain constant regions of kappa or lambda, or a fragment thereof. In some embodiments, the first anti-IMC antibody molecule comprises a kappa light chain constant region, or a fragment thereof, and the second anti-IMC antibody molecule comprises a lambda light chain constant region, or a fragment thereof. In some embodiments, the first anti-IMC antibody molecule comprises a lambda light chain constant region, or a fragment thereof, and the second anti-IMC antibody molecule comprises a kappa light chain constant region, or a fragment thereof. In some embodiments, the first anti-IMC antibody molecule and the second anti-IMC antibody molecule have a common light chain variable region.

In some embodiments the multispecific molecule further comprises a heavy chain constant region (e.g., an Fc region) chosen from the heavy chain constant regions of IgG1, IgG2, and IgG4, more particularly, the heavy chain constant region of human IgG1, IgG2 or IgG4. In some embodiments, the heavy chain constant region (e.g., an Fc region) is linked to, e.g., covalently linked to, one or both of the first anti-IMC antibody molecule and the second anti-IMC antibody molecule. In some embodiments, the heavy chain constant region (e.g., an Fc region) is altered, e.g., mutated, to increase or decrease one or more of: Fc receptor binding, antibody glycosylation, the number of cysteine residues, effector cell function, or complement function. In some embodiments, an interface of a first and second heavy chain constant regions (e.g., Fc region) is altered, e.g., mutated, to increase or decrease dimerization, e.g., relative to a non-engineered interface. In some embodiments, the dimerization of the heavy chain constant region (e.g., Fc region) is enhanced by providing an Fc interface of a first and a second Fc region with one or more of: a paired cavity-protuberance (“knob-in-a hole”), an electrostatic interaction, or a strand-exchange, such that a greater ratio of heteromultimer:homomultimer forms, e.g., relative to a non-engineered interface. In some embodiments, the heavy chain constant region (e.g., Fc region) comprises an amino acid substitution at a position chosen from one or more of 347, 349, 350, 351, 366, 368, 370, 392, 394, 395, 397, 398, 399, 405, 407, or 409, e.g., of the Fc region of human IgG1, numbered based on the Eu numbering system. In some embodiments, the heavy chain constant region (e.g., Fc region) comprises an amino acid substitution chosen from: T366S, L368A, or Y407V (e.g., corresponding to a cavity or hole), or T366W (e.g., corresponding to a protuberance or knob), or a combination thereof, numbered based on the Eu numbering system.

In some embodiments, the heavy chain constant region (e.g., an Fc region) comprises one or more mutations that increase or decrease one or more of: Fc receptor binding, antibody glycosylation, the number of cysteine residues, effector cell function, or complement function, relative to a naturally-existing heavy chain constant region. In some embodiments, the first anti-IMC antibody molecule comprises a first heavy chain constant region (e.g., a first Fc region) and the second anti-IMC antibody molecule comprises a second heavy chain constant region (e.g., a second Fc region), wherein the first heavy chain constant region comprises one or more mutations that increase heterodimerization of the first heavy chain constant region and the second heavy chain constant region, relative to a naturally-existing heavy chain constant region, and/or wherein the second heavy chain constant region comprises one or more mutations that increase heterodimerization of the second heavy chain constant region and the first heavy chain constant region, relative to a naturally-existing heavy chain constant region. In some embodiments, the first and the second heavy chain constant regions (e.g., first and second Fc regions) comprise one or more of: a paired cavity-protuberance (“knob-in-a hole”), an electrostatic interaction, or a strand-exchange, such that a greater ratio of heteromultimer:homomultimer forms, e.g., relative to naturally-existing heavy chain constant regions. In some embodiments, the first and/or second heavy chain constant region (e.g., a first and/or second Fc region, e.g., a first and/or second IgG1 Fc region) comprises an amino acid substitution at a position chosen from one or more of 347, 349, 350, 351, 366, 368, 370, 392, 394, 395, 397, 398, 399, 405, 407, or 409, numbered based on the Eu numbering system. In some embodiments, the first and/or second heavy chain constant region (e.g., a first and/or second Fc region, e.g., a first and/or second IgG1 Fc region) comprises an amino acid substitution chosen from: T366S, L368A, Y407V, or Y349C (e.g., corresponding to a cavity or hole), or T366W or S354C (e.g., corresponding to a protuberance or knob), or a combination thereof, numbered based on the Eu numbering system.

In some embodiments, the multispecific molecule further comprises a linker, e.g., a linker between one or more of: the first anti-IMC antibody molecule and the second anti-IMC antibody molecule, the first anti-IMC antibody molecule and the heavy chain constant region (e.g., the Fc region), or the second anti-IMC antibody molecule and the heavy chain constant region. In some embodiments, the linker is chosen from: a cleavable linker, a non-cleavable linker, a peptide linker, a flexible linker, a rigid linker, a helical linker, or a non-helical linker. In some embodiments, the linker is a peptide linker. In some embodiments, the peptide linker comprises Gly and Ser.

In some embodiments, the heavy chain constant region (e.g., Fc region) induces antibody dependent cellular cytotoxicity (ADCC).

In some embodiments, the first or the second TAM binding moiety is an antibody molecule that binds to CSF1R and comprises one, two, or three CDRs from the heavy chain variable region sequence of: SEQ ID NO: 48, SEQ ID NO: 66, or SEQ ID NO: 69, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 48, SEQ ID NO: 66, or SEQ ID NO: 69; and/or comprises one, two, or three CDRs from the light chain variable region sequence of: SEQ ID NO: 50, SEQ ID NO: 67, or SEQ ID NO: 70, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 50, SEQ ID NO: 67, or SEQ ID NO: 70. In some embodiments, the antibody molecule that binds to CSF1R comprises the heavy chain variable region sequence of: SEQ ID NO: 48, SEQ ID NO: 66, or SEQ ID NO: 69, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 48, SEQ ID NO: 66, or SEQ ID NO: 69; and/or comprises the light chain variable region sequence of: SEQ ID NO: 50, SEQ ID NO: 67, or SEQ ID NO: 70, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 50, SEQ ID NO: 67, or SEQ ID NO: 70.

In some embodiments, the first or the second TAM binding moiety is an antibody molecule that binds to CCR2 and comprises one, two, or three CDRs from the heavy chain variable region sequence of: SEQ ID NO: 44, SEQ ID NO: 54, SEQ ID NO: 59, SEQ ID NO: 62, SEQ ID NO: 64, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 44, SEQ ID NO: 54, SEQ ID NO: 59, SEQ ID NO: 62, SEQ ID NO: 64; and/or comprises one, two, or three CDRs from the light chain variable region sequence of: SEQ ID NO: 45, SEQ ID NO: 57, SEQ ID NO: 60, SEQ ID NO: 63, SEQ ID NO: 65, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 45, SEQ ID NO: 57, SEQ ID NO: 60, SEQ ID NO: 63, SEQ ID NO: 65. In some embodiments, the antibody molecule that binds to CCR2 comprises the heavy chain variable region sequence of: SEQ ID NO: 44, SEQ ID NO: 54, SEQ ID NO: 59, SEQ ID NO: 62, SEQ ID NO: 64, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 44, SEQ ID NO: 54, SEQ ID NO: 59, SEQ ID NO: 62, SEQ ID NO: 64; and/or comprises the light chain variable region sequence of: SEQ ID NO: 45, SEQ ID NO: 57, SEQ ID NO: 60, SEQ ID NO: 63, SEQ ID NO: 65, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 45, SEQ ID NO: 57, SEQ ID NO: 60, SEQ ID NO: 63, SEQ ID NO: 65.

In one embodiment, the first TAM binding moiety is an antibody molecule that binds to CCR2 and comprises one, two, or three CDRs from the heavy chain variable region sequence of: SEQ ID NO: 44, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 44; and/or comprises one, two, or three CDRs from the light chain variable region sequence of: SEQ ID NO: 45, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 45; and the second TAM binding moiety is an antibody molecule that binds to CSF1R and comprises one, two, or three CDRs from the heavy chain variable region sequence of: SEQ ID NO: 48, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 48; and/or comprises one, two, or three CDRs from the light chain variable region sequence of: SEQ ID NO: 50, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 50

In one embodiment, the first TAM binding moiety is an antibody molecule that binds to CCR2 and comprises one, two, or three CDRs from the heavy chain variable region sequence of: SEQ ID NO: 54, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 54; and/or comprises one, two, or three CDRs from the light chain variable region sequence of: SEQ ID NO: 57, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 57; and the second TAM binding moiety is an antibody molecule that binds to CSF1R and comprises one, two, or three CDRs from the heavy chain variable region sequence of: SEQ ID NO: 66, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 66; and/or comprises one, two, or three CDRs from the light chain variable region sequence of: SEQ ID NO: 67, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 67

In one embodiment, the first TAM binding moiety is an antibody molecule that binds to CCR2 and comprises one, two, or three CDRs from the heavy chain variable region sequence of: SEQ ID NO: 54, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 54; and/or comprises one, two, or three CDRs from the light chain variable region sequence of: SEQ ID NO: 57, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 57; and the second TAM binding moiety is an antibody molecule that binds to CSF1R and comprises one, two, or three CDRs from the heavy chain variable region sequence of: SEQ ID NO: 69, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 69; and/or comprises one, two, or three CDRs from the light chain variable region sequence of: SEQ ID NO: 70, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 70.

In one embodiment, the first TAM binding moiety is an antibody molecule that binds to CCR2 and comprises one, two, or three CDRs from the heavy chain variable region sequence of: SEQ ID NO: 59, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 59; and/or comprises one, two, or three CDRs from the light chain variable region sequence of: SEQ ID NO: 60, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 60; and the second TAM binding moiety is an antibody molecule that binds to CSF1R and comprises one, two, or three CDRs from the heavy chain variable region sequence of: SEQ ID NO: 66, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 66; and/or comprises one, two, or three CDRs from the light chain variable region sequence of: SEQ ID NO: 67, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 67.

In one embodiment, the first TAM binding moiety is an antibody molecule that binds to CCR2 and comprises one, two, or three CDRs from the heavy chain variable region sequence of: SEQ ID NO: 59, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 59; and/or comprises one, two, or three CDRs from the light chain variable region sequence of: SEQ ID NO: 60, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 60; and the second TAM binding moiety is an antibody molecule that binds to CSF1R and comprises one, two, or three CDRs from the heavy chain variable region sequence of: SEQ ID NO: 69, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 69; and/or comprises one, two, or three CDRs from the light chain variable region sequence of: SEQ ID NO: 70, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 70.

In one embodiment, the first TAM binding moiety is an antibody molecule that binds to CCR2 and comprises one, two, or three CDRs from the heavy chain variable region sequence of: SEQ ID NO: 62, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 62; and/or comprises one, two, or three CDRs from the light chain variable region sequence of: SEQ ID NO: 63, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 63; and the second TAM binding moiety is an antibody molecule that binds to CSF1R and comprises one, two, or three CDRs from the heavy chain variable region sequence of: SEQ ID NO: 66, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 66; and/or comprises one, two, or three CDRs from the light chain variable region sequence of: SEQ ID NO: 67, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 67.

In one embodiment, the first TAM binding moiety is an antibody molecule that binds to CCR2 and comprises one, two, or three CDRs from the heavy chain variable region sequence of: SEQ ID NO: 62, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 62; and/or comprises one, two, or three CDRs from the light chain variable region sequence of: SEQ ID NO: 63, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 63; and the second TAM binding moiety is an antibody molecule that binds to CSF1R and comprises one, two, or three CDRs from the heavy chain variable region sequence of: SEQ ID NO: 69, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 69; and/or comprises one, two, or three CDRs from the light chain variable region sequence of: SEQ ID NO: 70, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 70.

In one embodiment, the first TAM binding moiety is an antibody molecule that binds to CCR2 and comprises one, two, or three CDRs from the heavy chain variable region sequence of: SEQ ID NO: 64, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 64; and/or comprises one, two, or three CDRs from the light chain variable region sequence of: SEQ ID NO: 65, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 65; and the second TAM binding moiety is an antibody molecule that binds to CSF1R and comprises one, two, or three CDRs from the heavy chain variable region sequence of: SEQ ID NO: 66, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 66; and/or comprises one, two, or three CDRs from the light chain variable region sequence of: SEQ ID NO: 67, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 67.

In one embodiment, the first TAM binding moiety is an antibody molecule that binds to CCR2 and comprises one, two, or three CDRs from the heavy chain variable region sequence of: SEQ ID NO: 64, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 64; and/or comprises one, two, or three CDRs from the light chain variable region sequence of: SEQ ID NO: 65, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 65; and the second TAM binding moiety is an antibody molecule that binds to CSF1R and comprises one, two, or three CDRs from the heavy chain variable region sequence of: SEQ ID NO: 69, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 69; and/or comprises one, two, or three CDRs from the light chain variable region sequence of: SEQ ID NO: 70, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 70

In one embodiment, the first TAM binding moiety is an antibody molecule that binds to CCR2 and comprises the heavy chain variable region sequence of: SEQ ID NO: 44, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 44; and/or comprises the light chain variable region sequence of: SEQ ID NO: 45, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 45; and the second TAM binding moiety is an antibody molecule that binds to CSF1R and comprises the heavy chain variable region sequence of: SEQ ID NO: 48, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 48; and/or comprises the light chain variable region sequence of: SEQ ID NO: 50, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 50.

In one embodiment, the first TAM binding moiety is an antibody molecule that binds to CCR2 and comprises the heavy chain variable region sequence of: SEQ ID NO: 54, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 54; and/or comprises the light chain variable region sequence of: SEQ ID NO: 57, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 57; and the second TAM binding moiety is an antibody molecule that binds to CSF1R and comprises the heavy chain variable region sequence of: SEQ ID NO: 66, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 66; and/or comprises the light chain variable region sequence of: SEQ ID NO: 67, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 67

In one embodiment, the first TAM binding moiety is an antibody molecule that binds to CCR2 and comprises the heavy chain variable region sequence of: SEQ ID NO: 54, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 54; and/or comprises the light chain variable region sequence of: SEQ ID NO: 57, or a an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 57; and the second TAM binding moiety is an antibody molecule that binds to CSF1R and comprises the heavy chain variable region sequence of: SEQ ID NO: 69, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 69; and/or comprises the light chain variable region sequence of: SEQ ID NO: 70, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 70

In one embodiment, the first TAM binding moiety is an antibody molecule that binds to CCR2 and comprises the heavy chain variable region sequence of: SEQ ID NO: 59, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 59; and/or comprises the light chain variable region sequence of: SEQ ID NO: 60, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 60; and the second TAM binding moiety is an antibody molecule that binds to CSF1R and comprises the heavy chain variable region sequence of: SEQ ID NO: 66, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 66; and/or comprises the light chain variable region sequence of: SEQ ID NO: 67, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 67.

In one embodiment, the first TAM binding moiety is an antibody molecule that binds to CCR2 and comprises the heavy chain variable region sequence of: SEQ ID NO: 59, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 59; and/or comprises the light chain variable region sequence of: SEQ ID NO: 60, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 60; and the second TAM binding moiety is an antibody molecule that binds to CSF1R and comprises the heavy chain variable region sequence of: SEQ ID NO: 69, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 69; and/or comprises the light chain variable region sequence of: SEQ ID NO: 70, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 70.

In one embodiment, the first TAM binding moiety is an antibody molecule that binds to CCR2 and comprises the heavy chain variable region sequence of: SEQ ID NO: 62, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 62; and/or comprises the light chain variable region sequence of: SEQ ID NO: 63, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 63; and the second TAM binding moiety is an antibody molecule that binds to CSF1R and comprises the heavy chain variable region sequence of: SEQ ID NO: 66, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 66; and/or comprises the light chain variable region sequence of: SEQ ID NO: 67, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 67

In one embodiment, the first TAM binding moiety is an antibody molecule that binds to CCR2 and comprises the heavy chain variable region sequence of: SEQ ID NO: 62, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 62; and/or comprises the light chain variable region sequence of: SEQ ID NO: 63, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 63; and the second TAM binding moiety is an antibody molecule that binds to CSF1R and comprises the heavy chain variable region sequence of: SEQ ID NO: 69, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 69; and/or comprises the light chain variable region sequence of: SEQ ID NO: 70, an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 70.

In one embodiment, the first TAM binding moiety is an antibody molecule that binds to CCR2 and comprises the heavy chain variable region sequence of: SEQ ID NO: 64, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 64; and/or comprises the light chain variable region sequence of: SEQ ID NO: 65, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 65; and the second TAM binding moiety is an antibody molecule that binds to CSF1R and comprises the heavy chain variable region sequence of: SEQ ID NO: 66, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 66; and/or comprises the light chain variable region sequence of: SEQ ID NO: 67, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 67.

In one embodiment, the first TAM binding moiety is an antibody molecule that binds to CCR2 and comprises the heavy chain variable region sequence of: SEQ ID NO: 64, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 64; and/or comprises the light chain variable region sequence of: SEQ ID NO: 65, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 65; and the second TAM binding moiety is an antibody molecule that binds to CSF1R and comprises the heavy chain variable region sequence of: SEQ ID NO: 69, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 69; and/or comprises the light chain variable region sequence of: SEQ ID NO: 70, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 70.

In some embodiments, the first or the second TAM binding moiety is an antibody molecule that binds to PD-L1 and comprises one, two, or three CDRs from the heavy chain variable region sequence of: SEQ ID NO: 109, SEQ ID NO: 111, or SEQ ID NO: 113, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 109, SEQ ID NO: 111, or SEQ ID NO: 113; and/or comprises one, two, or three CDRs from the light chain variable region sequence of: SEQ ID NO: 110, SEQ ID NO: 112, or SEQ ID NO: 114, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 110, SEQ ID NO: 112, or SEQ ID NO: 114 In some embodiments, the antibody molecule that binds to PD-L1 comprises the heavy chain variable region sequence of: SEQ ID NO: 109, SEQ ID NO: 111, or SEQ ID NO: 113, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 109, SEQ ID NO: 111, or SEQ ID NO: 113); and/or comprises the light chain variable region sequence of: SEQ ID NO: 110, SEQ ID NO: 112, or SEQ ID NO: 114, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 110, SEQ ID NO: 112, or SEQ ID NO: 114).

In some embodiments, (i) the first IMC binding moiety binds to a first antigen (e.g., CSF1R, CCR2, CXCR2, CD86, CD163, CX3CR1, MARCO, CD204, CD52, folate receptor beta, or PD-L1) monovalently, and/or (ii) the second IMC binding moiety binds to a second antigen (e.g., CCR2, CSF1R, CXCR2, CD86, CD163, CX3CR1, MARCO, CD204, CD52, folate receptor beta, or PD-L1) monovalently, wherein the first antigen is different from the second antigen.

In some embodiments, (i) the multispecific molecule binds to a first antigen (e.g., CSF1R, CCR2, CXCR2, CD86, CD163, CX3CR1, MARCO, CD204, CD52, folate receptor beta, or PD-L1) monovalently, and/or (ii) the multispecific molecule binds to a second antigen (e.g., CCR2, CSF1R, CXCR2, CD86, CD163, CX3CR1, MARCO, CD204, CD52, folate receptor beta, or PD-L1) monovalently, wherein the first antigen is different from the second antigen.

In some embodiments, (i) the multispecific molecule inhibits a first antigen in the presence of a second antigen, optionally wherein the multispecific molecule reduces an activity of the first antigen in a cell, e.g., by at least 40, 50, 60, 70, 80, or 90%, when the cell expresses both the first antigen and the second antigen on the cell surface, and/or (ii) the multispecific molecule does not inhibit or does not substantially inhibit the first antigen in the absence of the second antigen, optionally wherein the multispecific molecule does not reduce an activity of the first antigen, or does not reduce an activity of the first antigen by more than 2, 4, 6, 8, 10, or 15%, when the cell expresses the first antigen but not the second antigen on the cell surface.

In some embodiments, (i) the multispecific molecule inhibits a second antigen in the presence of a first antigen, optionally wherein the multispecific molecule reduces an activity of the second antigen in a cell, e.g., by at least 40, 50, 60, 70, 80, or 90%, when the cell expresses both the first antigen and the second antigen on the cell surface, and/or (ii) the multispecific molecule does not inhibit or does not substantially inhibit the second antigen in the absence of the first antigen, optionally wherein the multispecific molecule does not reduce an activity of the second antigen, or does not reduce an activity of the second antigen by more than 2, 4, 6, 8, 10, or 15%, when the cell expresses the second antigen but not the first antigen on the cell surface.

In some embodiments, the multispecific molecule further comprises one or more additional binding moieties (e.g., a third binding moiety, a fourth binding moiety, (e.g., a trispecific or a tetraspecific molecule). In some embodiments, the multispecific molecule further comprises one or more additional binding moieties (e.g., a third binding moiety, a fourth binding moiety, (e.g., a trispecific or a tetraspecific molecule). In some embodiments, the multispecific molecule comprises a third TAM binding moiety (e.g., an antibody molecule), wherein the third TAM binding moiety is different from the first and the second TAM binding moieties. In some embodiments, the first TAM binding moiety binds to human CSF1R, the second TAM binding moiety binds to human CCR2, and the third TAM binding moiety binds to CXCR2.

In some embodiments, the multispecific molecule comprises a third binding moiety (e.g., antibody molecule) that is a tumor targeting moiety. In some embodiments, the tumor targeting moiety binds to PD-L1, mesothelin, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDCl27, CD47, α actinin-4, TRP1/gp75, TRP2, gp100, Melan-A/MART1, gangliosides, WT1, EphA3, Epidermal growth factor receptor (EGFR), CD20, MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, EGFRvIII, gpA33, GD3, GM2, VEGFR, Intergrins (Integrin alphaVbeta3, Integrin alpha5Beta1), Carbohydrates (Le), IGF1R, EPHA3, TRAILR1, TRAILR2, or RANKL.

In some embodiments, the multispecific molecule is a bispecific molecule comprising a first and a second non-contiguous polypeptides, wherein: (i) the first polypeptide includes, e.g., in the N- to C-orientation, the first TAM binding moiety (e.g., an antibody molecule (e.g., a first portion of a first antigen domain, e.g., a first VH-CH1 of a Fab molecule)), that binds to, e.g., a first TAM antigen, e.g., CSF1R, CCR2, CXCR2, or PD-L1, connected, optionally via a linker to, a first domain that promotes association between the first and the second polypeptide (e.g., a first immunoglobulin constant domain (e.g., a first Fc molecule as described herein); (ii) the second polypeptide includes, e.g., in the N- to C-orientation, the second TAM binding moiety (e.g., an antibody molecule, e.g., a scFv that binds to, e.g., a second TAM antigen, e.g., CCR2, CSF1R, CXCR2, or PD-L1)), connected, optionally, via a linker to, a second domain that promotes association between the first and the second polypeptide (e.g., a second immunoglobulin constant domain (e.g., a second Fc molecule as described herein); and (iii) the third polypeptide includes, e.g., in the N- to C-orientation, a second portion of the first antigen domain, e.g., a first VL-CL of the Fab, that binds to the first TAM antigen, e.g., wherein the third polypeptide associates non-covalently to the first polypeptide; and (iv) the fourth polypeptide includes, e.g., in the N- to C-orientation, a second portion of the second antigen domain, e.g., a second VL-CL of the Fab, that binds to the second TAM antigen, e.g., wherein the fourth polypeptide associates non-covalently to the second polypeptide. In some embodiments, the first and the second polypeptides are homo- or heterodimers.

In some embodiments, the multispecific molecule is a bispecific molecule, wherein:

(i) the first TAM binding moiety (e.g., a binding moiety that binds to a first TAM antigen, e.g., CSF1R, CCR2, or CXCR2) comprises a first and a second non-contiguous polypeptides, and

(ii) the second TAM binding moiety (e.g., a binding moiety that binds to a second TAM antigen, e.g., CSF1R, CCR2, or CXCR2) comprises a third and a fourth non-contiguous polypeptides, wherein:

(a) the first polypeptide comprises, e.g., in the N- to C-orientation, a first VH, a first CH1, connected, optionally via a linker, to a first domain (e.g., a first Fc region) that promotes association between the first and the third polypeptides,

(b) the second polypeptide comprises, e.g., in the N- to C-orientation, a first VL and a first CL,

(c) the third polypeptide comprises, e.g., in the N- to C-orientation, a second VH, a second CH1, connected, optionally via a linker, to a second domain (e.g., a second Fc region) that promotes association between the first and the third polypeptides, and

(d) the fourth polypeptide comprises, e.g., in the N- to C-orientation, a second VL and a second CL. In some embodiments, the first and the second domains (e.g., the first and the second Fc regions) form a homo- or heterodimer.

In one aspect, the invention provides an isolated multispecific, e.g., a bispecific, molecule, comprising (i) an anti-CSF1R binding moiety (e.g., an anti-CSF1R antibody molecule); and (ii) an anti-CCR2 binding moiety (e.g., an anti-CCR2 antibody molecule). Without wishing to be bound by theory, the anti-CSF1R/anti-CCR2 multispecific molecule may preferentially bind to a CSF1R-positive, CCR2-positive cell relative to a CSF1R-positive, CCR2-negative cell, or a CSF1R-negative, CCR2-positive cell. Exemplary CSF1R-positive, CCR2-positive cell include, but are not limited to, tumor-associated macrophages (TAMs) and myeloid derived suppressor cells (MDSCs). Exemplary CSF1R-positive, CCR2-negative cells include, but are not limited to, tissue-resident macrophages (e.g., Kupffer cells), and Langerhans cells. Exemplary CSF1R-negative, CCR2-positive cells include, but are not limited to, T cells (e.g., activated T cells, e.g., activated CD4+ and/or CD8+ T cells), NK cells, and neutrophils. Without wishing to be bound by theory, the anti-CSF1R/anti-CCR2 multispecific molecule may preferentially bind to CSF1R-positive, CCR2-positive cells (e.g., pro-tumorigenic TAMs or MDSCs) relative to CSF1R-positive, CCR2-negative cells (e.g., tissue-resident macrophages (e.g., Kupffer cells), or Langerhans cells), or CSF1R-negative, CCR2-positive cells (e.g., activated T cells or NK cells).

Without wishing to be bound by theory, pro-tumorigenic tumor associated macrophages (TAMs) and myeloid-derived suppressor cells (MDSC) express both CSF1R and CCR2. In contrast, Kupffer cells and other tissue macrophages express CSF1R but not CCR2. In one embodiment, the anti-CSF1R/anti-CCR2 multispecific antibody molecule disclosed herein selectively binds to intratumoral M-MDSCs and M2 macrophages. In one embodiment, the anti-CSF1R/anti-CCR2 multispecific antibody molecule disclosed herein reduces immunosuppressive myeloid cells and/or increases infiltration of cytotoxic T cells in the tumor. In one embodiment, the anti-CSF1R/anti-CCR2 multispecific antibody molecule disclosed herein depletes TAMs but spares healthy liver Kupffer cells. In one embodiment, the anti-CSF1R/anti-CCR2 multispecific antibody molecule disclosed herein is localized selectively to the tumor to reduce systemic immunotoxicity.

In some embodiments, the anti-CSF1R/anti-CCR2 multispecific molecule, when it binds to a target cell, may induce antibody-dependent cellular cytotoxicity (ADCC) or complement-dependent cytotoxicity (CDC) of the target cell. In some embodiments, the anti-CSF1R/anti-CCR2 multispecific molecule may preferentially bind to and reduce the number of immunosuppressive myeloid cells in the tumor microenvironment (e.g., TAMs or MDSCs), while sparing homeostatic myeloid cells (e.g., tissue-resident macrophages (e.g., Kupffer cells)) and other anti-tumor immune cells (e.g., activated T cells and NK cells). Depletion of homeostatic myeloid cells may be partially responsible for adverse events in patients receiving anti-CSF1R antibody therapies.

In some embodiments, the multispecific molecule has one or more (e.g., 1, 2, 3, 4, 5, 6, 7, 8, 9, 10, 11, 12, 13, or more) of the following properties:

(i) the multispecific molecule preferentially binds to a CSF1R-positive, CCR2-positive cell relative to a CSF1R-positive, CCR2-negative cell, or a CSF1R-negative, CCR2-positive cell, e.g., the binding of the multispecific molecule to the CSF1R-positive, CCR2-positive cell is at least 2, 4, 6, 8, 10, 15, 20, or 25-fold stronger than the binding of the multispecific molecule to the CSF1R-positive, CCR2-negative cell, or the CSF1R-negative, CCR2-positive cell, e.g., as measured using a flow cytometry analysis, e.g., as measured using methods described in Example 2 with respect to FIG. 1;

(ii) the multispecific molecule preferentially binds to a CSF1R-positive, CCR2-positive cell relative to a CSF1R-positive, CCR2-negative cell, e.g., the EC50 of the multispecific molecule for binding to a CSF1R-positive, CCR2-positive cell is no more than 60, 50, 40, 30, 20, or 10% of the EC50 of the multispecific molecule for binding to a CSF1R-positive, CCR2-negative cell, e.g., as measured using a flow cytometry analysis, e.g., as measured using methods described in Example 2 with respect to FIG. 1;

(iii) the multispecific molecule preferentially binds to a CSF1R-positive, CCR2-positive cell relative to a CSF1R-negative, CCR2-positive cell, e.g., the EC50 of the multispecific molecule for binding to a CSF1R-positive, CCR2-positive cell is no more than 50, 40, 30, 20, 10, or 5% of the EC50 of the multispecific molecule for binding to a CSF1R-negative, CCR2-positive cell, e.g., as measured using a flow cytometry analysis, e.g., as measured using methods described in Example 2 with respect to FIG. 1;

(iv) the multispecific molecule preferentially binds to tumor-associated macrophages (TAMs) or myeloid derived suppressor cells (MDSCs) relative to T cells, NK cells, neutrophils, tissue-resident macrophages (e.g., Kupffer cells), or Langerhans cells, e.g., the binding of the multispecific molecule to TAMs or MDSCs is at least 2, 4, 6, 8, 10, 15, 20, or 25-fold stronger than the binding of the multispecific molecule to T cells, NK cells, neutrophils, tissue-resident macrophages (e.g., Kupffer cells), or Langerhans cells, e.g., as measured using a flow cytometry analysis, e.g., as measured using methods described in Example 6 with respect to FIG. 5;

(v) the multispecific molecule inhibits monocyte migration, e.g., monocyte chemoattractant protein 1 (MCP1)-induced monocyte migration, e.g., reduces MCP1-induced monocyte migration by at least 40, 50, 60, or 70%, e.g., as measured using a transwell plate migration assay, e.g., as measured using methods described in Example 3 with respect to FIG. 2;

(vi) the multispecific molecule inhibits the proliferation of macrophages, e.g., bone marrow-derived macrophages, e.g., CSF-1-induced proliferation of bone marrow-derived macrophages, e.g., reduces CSF-1-induced proliferation of bone marrow-derived macrophages by at least 50, 60, 70, or 80%, e.g., as measured using a cell proliferation MTT assay, e.g., as measured using methods described in Example 4 with respect to FIG. 3B;

(vii) the multispecific molecule does not inhibit or does not substantially inhibit the differentiation of monocytes, e.g., bone marrow-derived monocytes, e.g., CSF-1-induced differentiation of bone marrow-derived monocytes, e.g., does not reduce CSF-1-induced differentiation of bone marrow-derived monocytes by more than 2, 4, 6, 8, or 10%, e.g., as measured using a flow cytometry analysis, e.g., as measured using methods described in Example 5 with respect to FIG. 4;

(viii) the multispecific molecule depletes suppressive myeloid cells, e.g., TAMs or MDSCs, e.g., reduces the number of suppressive myeloid cells, e.g., TAMs or MDSCs, by at least 80, 85, 90, 95, 99, or 99.5%, in vivo, e.g., as measured using a flow cytometry analysis, e.g., as measured using methods described in Example 7 with respect to FIG. 6;

(ix) the multispecific molecule does not deplete or does not substantially deplete tissue-resident macrophages, e.g., Kupffer cells, e.g., does not reduce the number of tissue-resident macrophages, e.g., Kupffer cells, by more than 4, 6, 8, 10, or 15%, in vivo, e.g., as measured using an immunohistochemistry analysis, e.g., as measured using methods described in Example 8 with respect to FIGS. 7B and 7D;

(x) the multispecific molecule increases CD86 or MHC class II expression on TAMs, e.g., as measured using a flow cytometry analysis or an immunohistochemistry analysis, e.g., as measured using methods described with respect to FIG. 21;

(xi) the multispecific molecule does not inhibit or does not substantially inhibit CSF-1 dependent cell survival of CSF1R-positive, CCR2-negative cells, e.g., does not reduce CSF-1 dependent cell survival of CSF1R-positive, CCR2-negative cells by more than 5, 10, or 15%, e.g., as measured using a cell viability MTT assay, e.g., as measured using methods described in Example 9 with respect to FIG. 8A;

(xii) the multispecific molecule increases CD8+ T cell tumor infiltration in vivo, e.g., increases % CD8+ T cells in CD3+ T cells in tumor by at least 1.5, 2, or 2.5-fold, e.g., as measured using a flow cytometry analysis, e.g., as measured using methods described in Example 10 with respect to FIG. 9;

(xiii) the multispecific molecule reduces Treg frequency in tumor in vivo, e.g., reduces Treg frequency in tumor by at least 15, 20, 25, or 30%, e.g., as measured using a flow cytometry analysis, e.g., as measured using methods described in Example 11 with respect to FIG. 10A;

(xiv) the multispecific molecule increases the CD8+ T cell/Treg ratio in tumor in vivo, e.g., increases the CD8+ T cell/Treg ratio in tumor by at least 2.5, 3, 3.5, 4, or 4.5-fold, e.g., as measured using a flow cytometry analysis, e.g., as measured using methods described in Example 11 with respect to FIG. 10B; or

(xv) the multispecific molecule reduces tumor growth, increases survival of a tumor-bearing animal, and/or enhances anti-tumor immune memory, e.g., as measured using methods described in Example 12 with respect to FIGS. 11A and 111B, or Example 19 with respect to FIG. 23.

In some embodiments, the multispecific molecule preferentially binds to a CSF1R-positive, CCR2-positive cell relative to a CSF1R-positive, CCR2-negative cell, or a CSF1R-negative, CCR2-positive cell, e.g., the binding of the multispecific molecule to the CSF1R-positive, CCR2-positive cell is at least 2, 4, 6, 8, 10, 15, 20, or 25-fold stronger than the binding of the multispecific molecule to the CSF1R-positive, CCR2-negative cell, or the CSF1R-negative, CCR2-positive cell, e.g., as measured using a flow cytometry analysis, e.g., as measured using methods described in Example 2 with respect to FIG. 1.

In some embodiments, the multispecific molecule preferentially binds to a CSF1R-positive, CCR2-positive cell relative to a CSF1R-positive, CCR2-negative cell, e.g., the EC50 of the multispecific molecule for binding to a CSF1R-positive, CCR2-positive cell is no more than 60, 50, 40, 30, 20, or 10% of the EC50 of the multispecific molecule for binding to a CSF1R-positive, CCR2-negative cell, e.g., as measured using a flow cytometry analysis, e.g., as measured using methods described in Example 2 with respect to FIG. 1.

In some embodiments, the multispecific molecule preferentially binds to a CSF1R-positive, CCR2-positive cell relative to a CSF1R-negative, CCR2-positive cell, e.g., the EC50 of the multispecific molecule for binding to a CSF1R-positive, CCR2-positive cell is no more than 50, 40, 30, 20, 10, or 5% of the EC50 of the multispecific molecule for binding to a CSF1R-negative, CCR2-positive cell, e.g., as measured using a flow cytometry analysis, e.g., as measured using methods described in Example 2 with respect to FIG. 1.

In some embodiments, the multispecific molecule preferentially binds to tumor-associated macrophages (TAMs) or myeloid derived suppressor cells (MDSCs) relative to T cells, NK cells, neutrophils, tissue-resident macrophages (e.g., Kupffer cells), or Langerhans cells, e.g., the binding of the multispecific molecule to TAMs or MDSCs is at least 2, 4, 6, 8, 10, 15, 20, or 25-fold stronger than the binding of the multispecific molecule to T cells, NK cells, neutrophils, tissue-resident macrophages (e.g., Kupffer cells), or Langerhans cells, e.g., as measured using a flow cytometry analysis, e.g., as measured using methods described in Example 6 with respect to FIG. 5.

In some embodiments, the multispecific molecule inhibits monocyte migration, e.g., monocyte chemoattractant protein 1 (MCP1)-induced monocyte migration, e.g., reduces MCP1-induced monocyte migration by at least 40, 50, 60, or 70%, e.g., as measured using a transwell plate migration assay, e.g., as measured using methods described in Example 3 with respect to FIG. 2.

In some embodiments, the multispecific molecule inhibits the proliferation of macrophages, e.g., bone marrow-derived macrophages, e.g., CSF-1-induced proliferation of bone marrow-derived macrophages, e.g., reduces CSF-1-induced proliferation of bone marrow-derived macrophages by at least 50, 60, 70, or 80%, e.g., as measured using a cell proliferation MTT assay, e.g., as measured using methods described in Example 4 with respect to FIG. 3B.

In some embodiments, the multispecific molecule does not inhibit or does not substantially inhibit the differentiation of monocytes, e.g., bone marrow-derived monocytes, e.g., CSF-1-induced differentiation of bone marrow-derived monocytes, e.g., does not reduce CSF-1-induced differentiation of bone marrow-derived monocytes by more than 2, 4, 6, 8, or 10%, e.g., as measured using a flow cytometry analysis, e.g., as measured using methods described in Example 5 with respect to FIG. 4.

In some embodiments, the multispecific molecule depletes suppressive myeloid cells, e.g., TAMs or MDSCs, e.g., reduces the number of suppressive myeloid cells, e.g., TAMs or MDSCs, by at least 80, 85, 90, 95, 99, or 99.5%, in vivo, e.g., as measured using a flow cytometry analysis, e.g., as measured using methods described in Example 7 with respect to FIG. 6.

In some embodiments, the multispecific molecule does not deplete or does not substantially deplete tissue-resident macrophages, e.g., Kupffer cells, e.g., does not reduce the number of tissue-resident macrophages, e.g., Kupffer cells, by more than 4, 6, 8, 10, or 15%, in vivo, e.g., as measured using an immunohistochemistry analysis, e.g., as measured using methods described in Example 8 with respect to FIGS. 7B and 7D.

In some embodiments, the multispecific molecule increases CD86 or MHC class II expression on TAMs, e.g., as measured using a flow cytometry analysis or an immunohistochemistry analysis, e.g., as measured using methods described with respect to FIG. 21.

In some embodiments, the multispecific molecule does not inhibit or does not substantially inhibit CSF-1 dependent cell survival of CSF1R-positive, CCR2-negative cells, e.g., does not reduce CSF-1 dependent cell survival of CSF1R-positive, CCR2-negative cells by more than 5, 10, or 15%, e.g., as measured using a cell viability MTT assay, e.g., as measured using methods described in Example 9 with respect to FIG. 8A.

In some embodiments, the multispecific molecule increases CD8+ T cell tumor infiltration in vivo, e.g., increases % CD8+ T cells in CD3+ T cells in tumor by at least 1.5, 2, or 2.5-fold, e.g., as measured using a flow cytometry analysis, e.g., as measured using methods described in Example 10 with respect to FIG. 9.

In some embodiments, the multispecific molecule reduces Treg frequency in tumor in vivo, e.g., reduces Treg frequency in tumor by at least 15, 20, 25, or 30%, e.g., as measured using a flow cytometry analysis, e.g., as measured using methods described in Example 11 with respect to FIG. 10A.

In some embodiments, the multispecific molecule increases the CD8+ T cell/Treg ratio in tumor in vivo, e.g., increases the CD8+ T cell/Treg ratio in tumor by at least 2.5, 3, 3.5, 4, or 4.5-fold, e.g., as measured using a flow cytometry analysis, e.g., as measured using methods described in Example 11 with respect to FIG. 10B.

In some embodiments, the multispecific molecule reduces tumor growth, increases survival of a tumor-bearing animal, and/or enhances anti-tumor immune memory, e.g., as measured using methods described in Example 12 with respect to FIGS. 11A and 111B, or Example 19 with respect to FIG. 23.

In some embodiments, the anti-CSF1R binding moiety and the anti-CCR2 binding moiety are, independently, a full antibody (e.g., an antibody that includes at least one, and preferably two, complete heavy chains, and at least one, and preferably two, complete light chains), or an antigen-binding fragment (e.g., a Fab, F(ab′)2, Fv, a scFv, a single domain antibody, or a diabody (dAb)). In some embodiments, the anti-CSF1R binding moiety and/or the anti-CCR2 binding moiety comprises a heavy chain constant region chosen from IgG1, IgG2, IgG3, or IgG4, or a fragment thereof. In some embodiments, the anti-CSF1R binding moiety and/or the anti-CCR2 binding moiety comprises a heavy chain constant region that can mediate antibody-dependent cellular cytotoxicity (ADCC) or complement-dependent cytotoxicity (CDC). In some embodiments, the anti-CSF1R binding moiety comprises a first heavy chain constant region (e.g., a first Fc region) and the anti-CCR2 binding moiety comprises a second heavy chain constant region (e.g., a second Fc region), wherein the first heavy chain constant region comprises one or more mutations that increase heterodimerization of the first heavy chain constant region and the second heavy chain constant region, relative to a naturally-existing heavy chain constant region, and/or wherein the second heavy chain constant region comprises one or more mutations that increase heterodimerization of the second heavy chain constant region and the first heavy chain constant region, relative to a naturally-existing heavy chain constant region. In some embodiments, the anti-CSF1R binding moiety and/or the anti-CCR2 binding moiety comprises a light chain constant region chosen from the light chain constant regions of kappa or lambda, or a fragment thereof. In some embodiments, the anti-CSF1R binding moiety comprises a kappa light chain constant region, or a fragment thereof, and the anti-CCR2 binding moiety comprises a lambda light chain constant region, or a fragment thereof. In some embodiments, the anti-CSF1R binding moiety comprises a lambda light chain constant region, or a fragment thereof, and the anti-CCR2 binding moiety comprises a kappa light chain constant region, or a fragment thereof. In some embodiments, the anti-CSF1R binding moiety and the anti-CCR2 binding moiety have a common light chain variable region. In some embodiments, the multispecific molecule further comprises a heavy chain constant region (e.g., an Fc region) chosen from the heavy chain constant regions of IgG1, IgG2, and IgG4, more particularly, the heavy chain constant region of human IgG1, IgG2 or IgG4. In some embodiments, the multispecific molecule further comprises a heavy chain constant region that can mediate antibody-dependent cellular cytotoxicity (ADCC) or complement-dependent cytotoxicity (CDC).

In some embodiments, the multispecific molecule comprises an anti-CSF1R antibody molecule and an anti-CCR2 antibody molecule, wherein:

(i) the anti-CSF1R antibody molecule comprises a first polypeptide and a second polypeptide, wherein the first polypeptide comprises a first light chain variable region (VL) and a first light chain constant region (CL), and the second polypeptide comprises a first heavy chain variable region (VH), a first heavy chain constant region 1 (CH1), and optionally, a first CH2 and a first CH3, and

(ii) the anti-CCR2 antibody molecule comprises a third polypeptide and a fourth polypeptide, wherein the third polypeptide comprises a second VL and a second CL, and the fourth polypeptide comprises a second VH, a second CH1, and optionally, a second CH2 and a second CH3.

In some embodiments, (i) the anti-CSF1R antibody molecule binds to CSF1R monovalently, and/or (ii) the anti-CCR2 antibody molecule binds to CCR2 monovalently. In some embodiments, the multispecific molecule binds to CSF1R monovalently, and/or binds to CCR2 monovalently. In some embodiments, the multispecific molecule binds to CSF1R monovalently, and binds to CCR2 monovalently.

In some embodiments,

(i) the multispecific molecule inhibits CSF1R in the presence of CCR2, optionally wherein the multispecific molecule reduces an activity of CSF1R (e.g., CSF1R signaling, e.g., CSF1-induced CSF1R signaling) in a cell, e.g., by at least 40, 50, 60, 70, 80, or 90%, when the cell expresses both CSF1R and CCR2 on the cell surface, and/or

(ii) the multispecific molecule does not inhibit or does not substantially inhibit CSF1R in the absence of CCR2, optionally wherein the multispecific molecule does not reduce an activity of CSF1R (e.g., CSF1R signaling, e.g., CSF1-induced CSF1R signaling), or does not reduce an activity of CSF1R by more than 2, 4, 6, 8, 10, or 15%, when the cell expresses CSF1R but not CCR2 on the cell surface.

In some embodiments,

(i) the multispecific molecule inhibits CCR2 in the presence of CSF1R, optionally wherein the multispecific molecule reduces an activity of CCR2 in a cell, e.g., by at least 40, 50, 60, 70, 80, or 90%, when the cell expresses both CCR2 and CSF1R on the cell surface, and/or

(ii) the multispecific molecule does not inhibit or does not substantially inhibit CCR2 in the absence of CSF1R, optionally wherein the multispecific molecule does not reduce an activity of CCR2, or does not reduce an activity of CCR2 by more than 2, 4, 6, 8, 10, or 15%, when the cell expresses CCR2 but not CSF1R on the cell surface.

In one aspect, this invention provides an isolated multispecific, e.g., a bispecific, molecule, comprising:

(i) a first binding moiety that binds to a molecule that mediates the trafficking of monocytes, e.g., inflammatory monocytes, e.g., Ly6C^{Hi} CCR2+CX3CR1^{Lo} inflammatory monocytes, optionally wherein the first binding moiety binds to CCR2; and

(ii) a second binding moiety that binds to a molecule that mediates the maturation and/or survival of monocytes and/or macrophages at an inflamed tissue, optionally wherein the second binding moiety binds to CSF1R.

In one aspect, this invention provides an isolated multispecific, e.g., a bispecific, molecule, comprising:

(i) a first binding moiety that reduces recruitment of inflammatory monocytes to tumor, optionally wherein the first binding moiety binds to and/or inhibits CCR2; and

(ii) a second binding moiety that reduces maturation and/or survival of monocytes and/or macrophages in the tumor microenvironment, optionally wherein the second binding moiety binds to and/or inhibits CSF1R.

In one aspect, this invention provides an isolated multispecific, e.g., a bispecific, molecule, comprising (i) an anti-CSF1R binding moiety (e.g., an anti-CSF1R antibody molecule); and (ii) an anti-PD-L1 binding moiety (e.g., an anti-PD-L1 antibody molecule).

In some embodiments of the aforementioned aspects and embodiments, the multispecific molecule further comprises one or more additional binding moieties (e.g., a third binding moiety, a fourth binding moiety, (e.g., a trispecific or a tetraspecific molecule), optionally wherein the third binding moiety is a third IMC binding moiety or a tumor targeting moiety. In some embodiments, the tumor targeting moiety is a tumor targeting moiety disclosed in WO2017165464, e.g., pages 108-118 of WO2017165464, herein incorporated by reference in its entirety.

In some embodiments of the aforementioned aspects and embodiments, the multispecific molecule further comprises an immune cell engager chosen from a T cell engager, an NK cell engager, a B cell engager, a dendritic cell engager, or a macrophage cell engager. In some embodiment, the immune cell engager is an immune cell engager disclosed in WO2017165464, e.g., pages 119-131 of WO2017165464, herein incorporated by reference in its entirety. In some embodiments, the immune cell engager binds to and activates an immune cell, e.g., an effector cell. In some embodiments, the immune cell engager binds to, but does not activate, an immune cell, e.g., an effector cell.

In some embodiments, the immune cell engager is a T cell engager, e.g., a T cell engager that mediates binding to and activation of a T cell, or a T cell engager that mediates binding to but not activation of a T cell. In some embodiments, the T cell engager binds to CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226, e.g., the T cell engager is an anti-CD3 antibody molecule.

In some embodiments, the immune cell engager is an NK cell engager, e.g., an NK cell engager that mediates binding to and activation of an NK cell, or an NK cell engager that mediates binding to but not activation of an NK cell. In some embodiments, the NK cell engager is chosen from an antibody molecule, e.g., an antigen binding domain, or ligand that binds to (e.g., activates): NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160.

In some embodiments, the immune cell engager is a B cell engager, e.g., a CD40L, an OX40L, or a CD70 ligand, or an antibody molecule that binds to OX40, CD40 or CD70. In some embodiments, the immune cell engager is a macrophage cell engager, e.g., a CD2 agonist; a CD40L; an OX40L; an antibody molecule that binds to OX40, CD40 or CD70; an agonist of a Toll-like receptor (TLR) (e.g., a TLR4, e.g., a constitutively active TLR4 (caTLR4) or a TLR9 agonist); CD47; or a STING agonist.

In some embodiments, the immune cell engager is a dendritic cell engager, e.g., a CD2 agonist, an OX40 antibody, an OX40L, 41BB agonist, a Toll-like receptor agonist or a fragment thereof (e.g., a TLR4, e.g., a constitutively active TLR4 (caTLR4)), CD47 agonist, or a STING agonist.

In some embodiments of the aforementioned aspects and embodiments, the multispecific molecule further comprises a cytokine molecule. In some embodiments, the cytokine molecule is chosen from interleukin-2 (IL-2), interleukin-7 (IL-7), interleukin-12 (IL-12), interleukin-15 (IL-15), interleukin-18 (IL-18), interleukin-21 (IL-21), or interferon gamma, or a fragment or variant thereof, or a combination of any of the aforesaid cytokines. In some embodiments, the cytokine molecule is a cytokine molecule disclosed in WO2017165464, e.g., pages 108-118 of WO2017165464, herein incorporated by reference in its entirety.

In some embodiments of the aforementioned aspects and embodiments, the multispecific molecule further comprises a stromal modifying moiety. In some embodiments, the stromal modifying moiety causes one or more of: decreases the level or production of a stromal or extracellular matrix (ECM) component; decreases tumor fibrosis; increases interstitial tumor transport; improves tumor perfusion; expands the tumor microvasculature; decreases interstitial fluid pressure (IFP) in a tumor; or decreases or enhances penetration or diffusion of an agent, e.g., a cancer therapeutic or a cellular therapy, into a tumor or tumor vasculature. In some embodiments, the stromal modifying moiety is a stromal modifying moiety disclosed in WO2017165464, e.g., pages 131-136 of WO2017165464, herein incorporated by reference in its entirety.

In one aspect, provided herein is a multispecific, e.g., a bispecific, molecule, comprising:

(i) an anti-CSF1R binding moiety (e.g., an anti-CSF1R antibody molecule);

(ii) an anti-CCR2 binding moiety (e.g., an anti-CCR2 antibody molecule); and one, two,

or all of:

(iii) a TGF-beta inhibitor;

(iv) an anti-PDL1 binding moiety (e.g., an anti-PDL1 antibody molecule); and

(v) a cytokine molecule, e.g., an IL-2 molecule.

In one embodiment, the multispecific molecule preferentially binds to a CSF1R-positive, CCR2-positive cell relative to a CSF1R-positive, CCR2-negative cell, or a CSF1R-negative, CCR2-positive cell, e.g., the binding of the multispecific molecule to the CSF1R-positive, CCR2-positive cell is at least 2, 4, 6, 8, 10, 15, 20, or 25-fold stronger than the binding of the multispecific molecule to the CSF1R-positive, CCR2-negative cell, or the CSF1R-negative, CCR2-positive cell, e.g., as measured using a flow cytometry analysis, e.g., as measured using methods described in Example 2 with respect to FIG. 1. In one embodiment, the multispecific molecule preferentially binds to a CSF1R-positive, CCR2-positive cell relative to a CSF1R-positive, CCR2-negative cell, e.g., the EC50 of the multispecific molecule for binding to a CSF1R-positive, CCR2-positive cell is no more than 60, 50, 40, 30, 20, or 10% of the EC50 of the multispecific molecule for binding to a CSF1R-positive, CCR2-negative cell, e.g., as measured using a flow cytometry analysis, e.g., as measured using methods described in Example 2 with respect to FIG. 1. In one embodiment, the multispecific molecule preferentially binds to a CSF1R-positive, CCR2-positive cell relative to a CSF1R-negative, CCR2-positive cell, e.g., the EC50 of the multispecific molecule for binding to a CSF1R-positive, CCR2-positive cell is no more than 50, 40, 30, 20, 10, or 5% of the EC50 of the multispecific molecule for binding to a CSF1R-negative, CCR2-positive cell, e.g., as measured using a flow cytometry analysis, e.g., as measured using methods described in Example 2 with respect to FIG. 1. In one embodiment, the multispecific molecule preferentially binds to tumor-associated macrophages (TAMs) or myeloid derived suppressor cells (MDSCs) relative to T cells, NK cells, neutrophils, tissue-resident macrophages (e.g., Kupffer cells), or Langerhans cells, e.g., the binding of the multispecific molecule to TAMs or MDSCs is at least 2, 4, 6, 8, 10, 15, 20, or 25-fold stronger than the binding of the multispecific molecule to T cells, NK cells, neutrophils, tissue-resident macrophages (e.g., Kupffer cells), or Langerhans cells, e.g., as measured using a flow cytometry analysis, e.g., as measured using methods described in Example 6 with respect to FIG. 5. In one embodiment, the multispecific molecule inhibits monocyte migration, e.g., monocyte chemoattractant protein 1 (MCP1)-induced monocyte migration, e.g., reduces MCP1-induced monocyte migration by at least 40, 50, 60, or 70%, e.g., as measured using a transwell plate migration assay, e.g., as measured using methods described in Example 3 with respect to FIG. 2. In one embodiment, the multispecific molecule inhibits the proliferation of macrophages, e.g., bone marrow-derived macrophages, e.g., CSF-1-induced proliferation of bone marrow-derived macrophages, e.g., reduces CSF-1-induced proliferation of bone marrow-derived macrophages by at least 50, 60, 70, or 80%, e.g., as measured using a cell proliferation MTT assay, e.g., as measured using methods described in Example 4 with respect to FIG. 3B. In one embodiment, the multispecific molecule does not inhibit or does not substantially inhibit the differentiation of monocytes, e.g., bone marrow-derived monocytes, e.g., CSF-1-induced differentiation of bone marrow-derived monocytes, e.g., does not reduce CSF-1-induced differentiation of bone marrow-derived monocytes by more than 2, 4, 6, 8, or 10%, e.g., as measured using a flow cytometry analysis, e.g., as measured using methods described in Example 5 with respect to FIG. 4. In one embodiment, the multispecific molecule depletes suppressive myeloid cells, e.g., TAMs or MDSCs, e.g., reduces the number of suppressive myeloid cells, e.g., TAMs or MDSCs, by at least 80, 85, 90, 95, 99, or 99.5%, in vivo, e.g., as measured using a flow cytometry analysis, e.g., as measured using methods described in Example 7 with respect to FIG. 6. In one embodiment, the multispecific molecule does not deplete or does not substantially deplete tissue-resident macrophages, e.g., Kupffer cells, e.g., does not reduce the number of tissue-resident macrophages, e.g., Kupffer cells, by more than 4, 6, 8, 10, or 15%, in vivo, e.g., as measured using an immunohistochemistry analysis, e.g., as measured using methods described in Example 8 with respect to FIGS. 7B and 7D. In one embodiment, the multispecific molecule increases CD86 or MHC class II expression on TAMs, e.g., as measured using a flow cytometry analysis or an immunohistochemistry analysis, e.g., as measured using methods described with respect to FIG. 21. In one embodiment, the multispecific molecule does not inhibit or does not substantially inhibit CSF-1 dependent cell survival of CSF1R-positive, CCR2-negative cells, e.g., does not reduce CSF-1 dependent cell survival of CSF1R-positive, CCR2-negative cells by more than 5, 10, or 15%, e.g., as measured using a cell viability MTT assay, e.g., as measured using methods described in Example 9 with respect to FIG. 8A. In one embodiment, the multispecific molecule increases CD8+ T cell tumor infiltration in vivo, e.g., increases % CD8+ T cells in CD3+ T cells in tumor by at least 1.5, 2, or 2.5-fold, e.g., as measured using a flow cytometry analysis, e.g., as measured using methods described in Example 10 with respect to FIG. 9. In one embodiment, the multispecific molecule reduces Treg frequency in tumor in vivo, e.g., reduces Treg frequency in tumor by at least 15, 20, 25, or 30%, e.g., as measured using a flow cytometry analysis, e.g., as measured using methods described in Example 11 with respect to FIG. 10A. In one embodiment, the multispecific molecule increases the CD8+ T cell/Treg ratio in tumor in vivo, e.g., increases the CD8+ T cell/Treg ratio in tumor by at least 2.5, 3, 3.5, 4, or 4.5-fold, e.g., as measured using a flow cytometry analysis, e.g., as measured using methods described in Example 11 with respect to FIG. 10B. In one embodiment, the multispecific molecule reduces tumor growth, increases survival of a tumor-bearing animal, and/or enhances anti-tumor immune memory, e.g., as measured using methods described in Example 12 with respect to FIGS. 11A and 111B, or Example 19 with respect to FIG. 23.

In one embodiment, the anti-CSF1R binding moiety and the anti-CCR2 binding moiety are, independently, a full antibody (e.g., an antibody that includes at least one, and preferably two, complete heavy chains, and at least one, and preferably two, complete light chains), or an antigen-binding fragment (e.g., a Fab, F(ab′)2, Fv, a scFv, a single domain antibody, or a diabody (dAb)). In one embodiment, the anti-CSF1R binding moiety and/or the anti-CCR2 binding moiety comprises a heavy chain constant region chosen from IgG1, IgG2, IgG3, or IgG4, or a fragment thereof. In one embodiment, the anti-CSF1R binding moiety and/or the anti-CCR2 binding moiety comprises a heavy chain constant region that can mediate antibody-dependent cellular cytotoxicity (ADCC) or complement-dependent cytotoxicity (CDC). In one embodiment, the anti-CSF1R binding moiety comprises a first heavy chain constant region (e.g., a first Fc region) and the anti-CCR2 binding moiety comprises a second heavy chain constant region (e.g., a second Fc region), wherein the first heavy chain constant region comprises one or more mutations that increase heterodimerization of the first heavy chain constant region and the second heavy chain constant region, relative to a naturally-existing heavy chain constant region, and/or wherein the second heavy chain constant region comprises one or more mutations that increase heterodimerization of the second heavy chain constant region and the first heavy chain constant region, relative to a naturally-existing heavy chain constant region. In one embodiment, the anti-CSF1R binding moiety and/or the anti-CCR2 binding moiety comprises a light chain constant region chosen from the light chain constant regions of kappa or lambda, or a fragment thereof. In one embodiment, the anti-CSF1R binding moiety comprises a kappa light chain constant region, or a fragment thereof, and the anti-CCR2 binding moiety comprises a lambda light chain constant region, or a fragment thereof. In one embodiment, the anti-CSF1R binding moiety comprises a lambda light chain constant region, or a fragment thereof, and the anti-CCR2 binding moiety comprises a kappa light chain constant region, or a fragment thereof. In one embodiment, the anti-CSF1R binding moiety and the anti-CCR2 binding moiety have a common light chain variable region. In one embodiment, the multispecific molecule further comprises a heavy chain constant region (e.g., an Fc region) chosen from the heavy chain constant regions of IgG1, IgG2, and IgG4, more particularly, the heavy chain constant region of human IgG1, IgG2 or IgG4. In one embodiment, the multispecific molecule further comprises a heavy chain constant region that can mediate antibody-dependent cellular cytotoxicity (ADCC) or complement-dependent cytotoxicity (CDC). In one embodiment, the multispecific molecule comprises an anti-CSF1R antibody molecule and an anti-CCR2 antibody molecule, wherein: (i) the anti-CSF1R antibody molecule comprises a first polypeptide and a second polypeptide, wherein the first polypeptide comprises a first light chain variable region (VL) and a first light chain constant region (CL), and the second polypeptide comprises a first heavy chain variable region (VH), a first heavy chain constant region 1 (CH1), and optionally, a first CH2 and a first CH3, and (ii) the anti-CCR2 antibody molecule comprises a third polypeptide and a fourth polypeptide, wherein the third polypeptide comprises a second VL and a second CL, and the fourth polypeptide comprises a second VH, a second CH1, and optionally, a second CH2 and a second CH3.

In one embodiment, the anti-CSF1R antibody molecule binds to CSF1R monovalently. In one embodiment, the anti-CCR2 antibody molecule binds to CCR2 monovalently. In one embodiment, the multispecific molecule binds to CSF1R monovalently. In one embodiment, the multispecific molecule binds to CCR2 monovalently. In one embodiment, the multispecific molecule binds to CSF1R monovalently, and binds to CCR2 monovalently.

In one embodiment, the multispecific molecule inhibits CSF1R in the presence of CCR2, optionally wherein the multispecific molecule reduces an activity of CSF1R (e.g., CSF1R signaling, e.g., CSF1-induced CSF1R signaling) in a cell, e.g., by at least 40, 50, 60, 70, 80, or 90%, when the cell expresses both CSF1R and CCR2 on the cell surface. In one embodiment, the multispecific molecule does not inhibit or does not substantially inhibit CSF1R in the absence of CCR2, optionally wherein the multispecific molecule does not reduce an activity of CSF1R (e.g., CSF1R signaling, e.g., CSF1-induced CSF1R signaling), or does not reduce an activity of CSF1R by more than 2, 4, 6, 8, 10, or 15%, when the cell expresses CSF1R but not CCR2 on the cell surface. In one embodiment, the multispecific molecule inhibits CCR2 in the presence of CSF1R, optionally wherein the multispecific molecule reduces an activity of CCR2 in a cell, e.g., by at least 40, 50, 60, 70, 80, or 90%, when the cell expresses both CCR2 and CSF1R on the cell surface. In one embodiment, the multispecific molecule does not inhibit or does not substantially inhibit CCR2 in the absence of CSF1R, optionally wherein the multispecific molecule does not reduce an activity of CCR2, or does not reduce an activity of CCR2 by more than 2, 4, 6, 8, 10, or 15%, when the cell expresses CCR2 but not CSF1R on the cell surface.

In one embodiment, the multispecific molecule comprises (i) an anti-CSF1R binding moiety (e.g., an anti-CSF1R antibody molecule); (ii) an anti-CCR2 binding moiety (e.g., an anti-CCR2 antibody molecule); and (iii) a TGF beta inhibitor. In one embodiment, the TGF beta inhibitor inhibits TGF-beta 1, TGF-beta 3, or both TGF-beta 1 and TGF-beta 3, e.g., as measured using the methods described in Example 20 with respect to FIG. 26B. In one embodiment, the TGF beta inhibitor inhibits TGF-beta 1, TGF-beta 2, TGF-beta 3, both TGF-beta 1 and TGF-beta 3, or TGF-beta 1, TGF-beta 2, and TGF-beta 3, e.g., as measured using the methods described in Example 20 with respect to FIG. 26B. In one embodiment, the multispecific molecule reduces tumor growth and/or increases survival of a tumor-bearing animal, e.g., as measured using methods described in Example 20 with respect to FIGS. 27A-27C. In one embodiment, the TGF beta inhibitor comprises a TGF-beta receptor polypeptide (e.g., an extracellular domain of a TGF-beta receptor, or a functional variant thereof). In one embodiment, the TGF-beta inhibitor comprises one, two, or all of: a TGFBR1 polypeptide (e.g., 1, 2, 3, or more of a TGFBR1 polypeptide), a TGFBR2 polypeptide (e.g., 1, 2, 3, or more of a TGFBR2 polypeptide), or a TGFBR3 polypeptide (e.g., 1, 2, 3, or more of a TGFBR3 polypeptide). In one embodiment, the TGF-beta inhibitor comprises a TGFBR1 polypeptide. In one embodiment, the TGF-beta inhibitor comprises an extracellular domain of TGFBR1 or a sequence substantially identical thereto (e.g., a sequence that is at least 80%, 85%, 90%, or 95% identical thereto). In one embodiment, the TGF-beta inhibitor comprises an extracellular domain of SEQ ID NO: 95, 96, 97, 120, 121, or 122, or a sequence substantially identical thereto (e.g., a sequence that is at least 80%, 85%, 90%, or 95% identical thereto). In one embodiment, the TGF-beta inhibitor comprises the amino acid sequence of SEQ ID NO: 104 or 105, or a sequence substantially identical thereto (e.g., a sequence that is at least 80%, 85%, 90%, or 95% identical thereto). In one embodiment, the TGF beta inhibitor comprises a TGFBR2 polypeptide. In one embodiment, the TGF-beta inhibitor comprises an extracellular domain of TGFBR2 or a sequence substantially identical thereto (e.g., a sequence that is 80%, 85%, 90%, or 95% identical thereto). In one embodiment, the TGF-beta inhibitor comprises an extracellular domain of SEQ ID NO: 98, 99, 123, or 124, or a sequence substantially identical thereto (e.g., a sequence that is 80%, 85%, 90%, or 95% identical thereto). In one embodiment, the TGF-beta inhibitor comprises an amino acid sequence selected from the group consisting of SEQ ID NOs: 100, 101, 102, and 103, or a sequence substantially identical thereto (e.g., a sequence that is at least 80%, 85%, 90%, or 95% identical thereto). In one embodiment, the TGF-beta inhibitor comprises a TGFBR3 polypeptide. In one embodiment, the TGF-beta inhibitor comprises an extracellular domain of TGFBR3 or a sequence substantially identical thereto (e.g., a sequence that is at least 80%, 85%, 90%, or 95% identical thereto). In one embodiment, the TGF-beta inhibitor comprises an extracellular domain of SEQ ID NO: 106, 107, 125, or 126, or a sequence substantially identical thereto (e.g., a sequence that is at least 80%, 85%, 90%, or 95% identical thereto). In one embodiment, the TGF-beta inhibitor comprises the amino acid sequence of SEQ ID NO: 108, or a sequence substantially identical thereto (e.g., a sequence that is at least 80%, 85%, 90%, or 95% identical thereto). In one embodiment, the TGF-beta inhibitor comprises two TGF-beta receptor polypeptides that form a homodimer. In one embodiment, the TGF-beta inhibitor comprises two TGFBR1 polypeptides that form a homodimer. In one embodiment, the TGF-beta inhibitor comprises two TGFBR2 polypeptides that form a homodimer. In one embodiment, the TGF-beta inhibitor comprises two TGFBR3 polypeptides that form a homodimer. In one embodiment, the TGF-beta inhibitor comprises two TGF-beta receptor polypeptides that form a heterodimer. In one embodiment, the TGF-beta inhibitor comprises a TGFBR1 polypeptide and a TGFBR2 polypeptide that form a heterodimer. In one embodiment, the TGF-beta inhibitor comprises a TGFBR1 polypeptide and a TGFBR3 polypeptide that form a heterodimer. In one embodiment, the TGF-beta inhibitor comprises a TGFBR2 polypeptide and a TGFBR3 polypeptide that form a heterodimer.

In one embodiment, the TGF-beta inhibitor comprises a first TGF-beta receptor polypeptide and a second TGF-beta receptor polypeptide. In one embodiment, the multispecific molecule comprises a first Fc region (e.g., a first CH1-Fc region) and a second Fc region (e.g., a second CH1-Fc region), optionally wherein: (i) the first TGF-beta receptor polypeptide is linked, e.g., via a linker, to the first Fc region (e.g., a first CH1-Fc region), e.g., the C-terminus of the first Fc region (e.g., a first CH1-Fc region), and (ii) the second TGF-beta receptor polypeptide is linked, e.g., via a linker, to the second Fc region (e.g., a second CH1-Fc region), e.g., the C-terminus of the second Fc region (e.g., a second CH1-Fc region). In one embodiment, the first TGF-beta receptor polypeptide and the second TGF-beta receptor polypeptide form a homodimer or heterodimer, e.g., a homodimer. In one embodiment, the first or second TGF-beta receptor polypeptide comprises an extracellular domain of TGFBR1, TGFBR2, or TGFBR3, e.g., an extracellular domain of TGFBR2. In one embodiment, the multispecific molecule has the configuration of FIG. 35A or 35B. In one embodiment, the multispecific molecule comprises the amino acid sequence of SEQ ID NO: 192 (or a sequence substantially identical thereto, e.g., a sequence that is at least 80%, 85%, 90%, or 95% identical thereto) and the amino acid sequence of SEQ ID NO: 193 (or a sequence substantially identical thereto, e.g., a sequence that is at least 80%, 85%, 90%, or 95% identical thereto). In one embodiment, the multispecific molecule comprises the amino acid sequence of SEQ ID NO: 192 (or a sequence substantially identical thereto, e.g., a sequence that is at least 80%, 85%, 90%, or 95% identical thereto) and the amino acid sequence of SEQ ID NO: 195 (or a sequence substantially identical thereto, e.g., a sequence that is at least 80%, 85%, 90%, or 95% identical thereto). In one embodiment, the multispecific molecule comprises the amino acid sequence of SEQ ID NO: 194 (or a sequence substantially identical thereto, e.g., a sequence that is at least 80%, 85%, 90%, or 95% identical thereto) and the amino acid sequence of SEQ ID NO: 193 (or a sequence substantially identical thereto, e.g., a sequence that is at least 80%, 85%, 90%, or 95% identical thereto). In one embodiment, the multispecific molecule comprises the amino acid sequence of SEQ ID NO: 194 (or a sequence substantially identical thereto, e.g., a sequence that is at least 80%, 85%, 90%, or 95% identical thereto) and the amino acid sequence of SEQ ID NO: 195 (or a sequence substantially identical thereto, e.g., a sequence that is at least 80%, 85%, 90%, or 95% identical thereto).

In one embodiment, the TGF-beta inhibitor comprises a first TGF-beta receptor polypeptide and a second TGF-beta receptor polypeptide. In one embodiment, the multispecific molecule comprises a heavy chain constant region 1 (CH1) and a light chain constant region (CL), optionally wherein: (i) the first TGF-beta receptor polypeptide is linked, e.g., via a linker, to the CH1, e.g., the N-terminus of the CH1, and (ii) the second TGF-beta receptor polypeptide is linked, e.g., via a linker, to the CL, e.g., the N-terminus of the CL. In one embodiment, the first TGF-beta receptor polypeptide and the second TGF-beta receptor polypeptide form a homodimer or heterodimer, e.g., a homodimer. In one embodiment, the first or second TGF-beta receptor polypeptide comprises an extracellular domain of TGFBR1, TGFBR2, or TGFBR3, e.g., an extracellular domain of TGFBR2. In one embodiment, the multispecific molecule has the configuration of FIG. 35C or 35D. In one embodiment, the multispecific molecule comprises the amino acid sequence of SEQ ID NO: 196 (or a sequence substantially identical thereto, e.g., a sequence that is at least 80%, 85%, 90%, or 95% identical thereto) and the amino acid sequence of SEQ ID NO: 198 (or a sequence substantially identical thereto, e.g., a sequence that is at least 80%, 85%, 90%, or 95% identical thereto). In one embodiment, the multispecific molecule comprises the amino acid sequence of SEQ ID NO: 196 (or a sequence substantially identical thereto, e.g., a sequence that is at least 80%, 85%, 90%, or 95% identical thereto) and the amino acid sequence of SEQ ID NO: 199 (or a sequence substantially identical thereto, e.g., a sequence that is at least 80%, 85%, 90%, or 95% identical thereto). In one embodiment, the multispecific molecule comprises the amino acid sequence of SEQ ID NO: 197 (or a sequence substantially identical thereto, e.g., a sequence that is at least 80%, 85%, 90%, or 95% identical thereto) and the amino acid sequence of SEQ ID NO: 198 (or a sequence substantially identical thereto, e.g., a sequence that is at least 80%, 85%, 90%, or 95% identical thereto). In one embodiment, the multispecific molecule comprises the amino acid sequence of SEQ ID NO: 197 (or a sequence substantially identical thereto, e.g., a sequence that is at least 80%, 85%, 90%, or 95% identical thereto) and the amino acid sequence of SEQ ID NO: 199 (or a sequence substantially identical thereto, e.g., a sequence that is at least 80%, 85%, 90%, or 95% identical thereto).

In one embodiment, the TGF inhibitor is linked, e.g., via a linker, to the anti-CSF1R binding moiety (e.g., an anti-CSF1R antibody molecule). In one embodiment, the TGF inhibitor is linked, e.g., via a linker, to the anti-CCR2 binding moiety (e.g., an anti-CCR2 antibody molecule). In one embodiment, the anti-CSF1R binding moiety and/or the anti-CCR2 binding moiety comprises an Fc region, wherein the TGF inhibitor is linked, e.g., via a linker, to the Fc region, e.g., the C-terminus of the Fc region. In one embodiment, the multispecific molecule comprises a first TGF-beta inhibitor and a second TGF-beta inhibitor, wherein the first TGF-beta inhibitor is linked, e.g., via a linker, to the anti-CSF1R binding moiety (e.g., an anti-CSF1R antibody molecule) and the second TGF-beta inhibitor is linked, e.g., via a linker, to the anti-CCR2 binding moiety (e.g., an anti-CCR2 antibody molecule). In one embodiment, the anti-CSF1R binding moiety comprises a first Fc region, the anti-CCR2 binding moiety comprises a second Fc region, and the multispecific molecule comprises a first TGF-beta inhibitor and a second TGF-beta inhibitor, wherein the first TGF-beta inhibitor is linked, e.g., via a linker, to the first Fc region, e.g., the C-terminus of the first Fc region, and the second TGF-beta inhibitor is linked, e.g., via a linker, to the second Fc region, e.g., the C-terminus of the second Fc region.

In one embodiment, the anti-CSF1R binding moiety comprises a first light chain and a first heavy chain, wherein the first heavy chain comprises a first Fc region, wherein the C-terminus of the first Fc region is linked to an extracellular domain of TGFBR2 or a sequence substantially identical thereto (e.g., a sequence that is at least 80%, 85%, 90%, or 95% identical thereto). In one embodiment, the anti-CCR2 binding moiety comprises a second light chain and a second heavy chain, wherein the second heavy chain comprises a second Fc region, wherein the C-terminus of the second Fc region is linked to an extracellular domain of TGFBR2 or a sequence substantially identical thereto (e.g., a sequence that is at least 80%, 85%, 90%, or 95% identical thereto). In one embodiment, the multispecific molecule has the configuration shown in FIG. 12B.

In one embodiment, the TGF inhibitor is linked, e.g., via a linker, to the anti-CSF1R binding moiety (e.g., an anti-CSF1R antibody molecule). In one embodiment, the TGF inhibitor is linked, e.g., via a linker, to the anti-CCR2 binding moiety (e.g., an anti-CCR2 antibody molecule). In one embodiment, the anti-CSF1R binding moiety and/or the anti-CCR2 binding moiety comprises an Fc region, wherein the TGF inhibitor is linked, e.g., via a linker, to the Fc region, e.g., the N-terminus of the Fc region.

In one embodiment, the multispecific molecule comprises (i) an anti-CSF1R binding moiety (e.g., an anti-CSF1R antibody molecule); (ii) an anti-CCR2 binding moiety (e.g., an anti-CCR2 antibody molecule); and (iii) an anti-PDL1 binding moiety (e.g., an anti-PDL1 antibody molecule). In one embodiment, the anti-PDL1 binding moiety inhibits PDL1. In one embodiment, the anti-PDL1 binding moiety comprises a heavy chain variable domain (VH) comprising a HCDR1, a HCDR2, and a HCDR3 of any VH sequence of Table 10, or a sequence having at least one amino acid alteration, but not more than two, three or four alterations (e.g., substitutions, deletions, or insertions, e.g., conservative substitutions). In one embodiment, the anti-PDL1 binding moiety comprises a VH comprising any VH sequence of Table 10, or a sequence substantially identical thereto, e.g., 95% to 99.9% identical thereto, or having at least one amino acid alteration, but not more than five, ten or fifteen alterations (e.g., substitutions, deletions, or insertions, e.g., conservative substitutions). In one embodiment, the anti-PDL1 binding moiety comprises a light chain variable domain (VL) comprising a LCDR1, a LCDR2, and a LCDR3 of any VL sequence of Table 10, or a sequence having at least one amino acid alteration, but not more than two, three or four alterations (e.g., substitutions, deletions, or insertions, e.g., conservative substitutions). In one embodiment, the anti-PDL1 binding moiety comprises a VL comprising any VL sequence of Table 10, or a sequence substantially identical thereto, e.g., 95% to 99.9% identical thereto, or having at least one amino acid alteration, but not more than five, ten or fifteen alterations (e.g., substitutions, deletions, or insertions, e.g., conservative substitutions).

In one embodiment, the anti-PDL1 binding moiety is linked, e.g., via a linker, to the anti-CSF1R binding moiety (e.g., an anti-CSF1R antibody molecule) or the anti-CCR2 binding moiety (e.g., an anti-CCR2 antibody molecule). In one embodiment, the anti-CSF1R binding moiety and/or the anti-CCR2 binding moiety comprises an Fc region, wherein the anti-PDL1 binding moiety is linked, e.g., via a linker, to the Fc region, e.g., the C-terminus of the Fc region. In one embodiment, the PDL1 binding moiety is an scFv. In one embodiment, the multispecific molecule has the configuration shown in FIG. 12C or 12D.

In one embodiment, the multispecific molecule comprises (i) an anti-CSF1R binding moiety (e.g., an anti-CSF1R antibody molecule); (ii) an anti-CCR2 binding moiety (e.g., an anti-CCR2 antibody molecule); and (iii) an IL-2 molecule. In one embodiment, the IL-2 molecule is an IL-2 molecule disclosed herein, e.g., an IL-2 molecule comprising the amino acid sequence of SEQ ID NO: 237 or 238, or a sequence substantially identical thereto (e.g., a sequence that is 80%, 85%, 90%, or 95% identical thereto).

In one embodiment, the IL-2 molecule is linked, e.g., via a linker, to the anti-CSF1R binding moiety (e.g., an anti-CSF1R antibody molecule) or the anti-CCR2 binding moiety (e.g., an anti-CCR2 antibody molecule). In one embodiment, the anti-CSF1R binding moiety and/or the anti-CCR2 binding moiety comprises a light chain constant region, wherein the IL-2 molecule is linked, e.g., via a linker, to the light chain constant region, e.g., the C-terminus of the light chain constant region. In one embodiment, the multispecific molecule comprises a first IL-2 molecule and a second IL-2 molecule, wherein the first IL-2 molecule is linked, e.g., via a linker, to the anti-CSF1R binding moiety (e.g., an anti-CSF1R antibody molecule) and the second IL-2 molecule is linked, e.g., via a linker, to the anti-CCR2 binding moiety (e.g., an anti-CCR2 antibody molecule). In one embodiment, the anti-CSF1R binding moiety comprises a first light chain constant region, the anti-CCR2 binding moiety comprises a second light chain constant region, and the multispecific molecule comprises a first IL-2 molecule and a second IL-2 molecule, wherein the first IL-2 molecule is linked, e.g., via a linker, to the first light chain constant region, e.g., the C-terminus of the first light chain constant region, and the second IL-2 molecule is linked, e.g., via a linker, to the second light chain constant region, e.g., the C-terminus of the second light chain constant region. In one embodiment, the multispecific molecule has the configuration shown in FIG. 12E, 12F, or 12G.

In one aspect, provided herein is an antibody molecule (e.g., an isolated antibody molecule) that binds to CSF1R (e.g., human CSF1R), comprising a heavy chain variable region (VH) comprising a heavy chain complementarity determining region 1 (HCDR1), a HCDR2, and a HCDR3, wherein the HCDR1, HCDR2, and HCDR3 comprise the amino acid sequences of SEQ ID NOs: 402, 474, and 475, respectively, or an amino acid sequence comprising no more than 1, 2, 3, 4, 5, or 6 modifications. In some embodiments, the antibody molecule further comprises a light chain variable region (VL) comprising a light chain complementarity determining region 1 (LCDR1), a LCDR2, and a LCDR3. In some embodiments, the LCDR1, LCDR2, and LCDR3 comprise the amino acid sequences of SEQ ID NOs: 432, 434, and 436, respectively, or an amino acid sequence comprising no more than 1, 2, 3, 4, 5, or 6 modifications. In some embodiments, the LCDR1, LCDR2, and LCDR3 comprise the amino acid sequences of SEQ ID NOs: 433, 435, and 437, respectively, or an amino acid sequence comprising no more than 1, 2, 3, 4, 5, or 6 modifications. In one aspect, provided herein is an antibody molecule (e.g., an isolated antibody molecule) that binds to CSF1R (e.g., human CSF1R), comprising a light chain variable region (VL) comprising a light chain complementarity determining region 1 (LCDR1), a LCDR2, and a LCDR3, wherein the LCDR1, LCDR2, and LCDR3 comprise the amino acid sequences of SEQ ID NOs: 432, 434, and 436, respectively, or SEQ ID NOs: 433, 435, and 437, respectively, or an amino acid sequence comprising no more than 1, 2, 3, 4, 5, or 6 modifications.

In some embodiments, the HCDR1, HCDR2, and HCDR3 comprise the amino acid sequences of SEQ ID NOs: 402, 476, and 477, respectively, or an amino acid sequence comprising no more than 1, 2, 3, 4, 5, or 6 modifications.

In some embodiments, the HCDR1, HCDR2, and HCDR3 comprise the amino acid sequences of: SEQ ID NOs: 402, 404, and 413, respectively, or an amino acid sequence comprising no more than 1, 2, 3, 4, 5, or 6 modifications; SEQ ID NOs: 402, 405, and 413, respectively, or an amino acid sequence comprising no more than 1, 2, 3, 4, 5, or 6 modifications; SEQ ID NOs: 402, 406, and 413, respectively, or an amino acid sequence comprising no more than 1, 2, 3, 4, 5, or 6 modifications; SEQ ID NOs: 402, 404, and 414, respectively, or an amino acid sequence comprising no more than 1, 2, 3, 4, 5, or 6 modifications; SEQ ID NOs: 402, 407, and 413, respectively, or an amino acid sequence comprising no more than 1, 2, 3, 4, 5, or 6 modifications; SEQ ID NOs: 402, 404, and 415, respectively, or an amino acid sequence comprising no more than 1, 2, 3, 4, 5, or 6 modifications; SEQ ID NOs: 402, 408, and 413, respectively, or an amino acid sequence comprising no more than 1, 2, 3, 4, 5, or 6 modifications; SEQ ID NOs: 402, 404, and 416, respectively, or an amino acid sequence comprising no more than 1, 2, 3, 4, 5, or 6 modifications; SEQ ID NOs: 402, 404, and 417, respectively, or an amino acid sequence comprising no more than 1, 2, 3, 4, 5, or 6 modifications; SEQ ID NOs: 402, 409, and 413, respectively, or an amino acid sequence comprising no more than 1, 2, 3, 4, 5, or 6 modifications; SEQ ID NOs: 402, 404, and 418, respectively, or an amino acid sequence comprising no more than 1, 2, 3, 4, 5, or 6 modifications; SEQ ID NOs: 402, 404, and 419, respectively, or an amino acid sequence comprising no more than 1, 2, 3, 4, 5, or 6 modifications; SEQ ID NOs: 402, 404, and 420, respectively, or an amino acid sequence comprising no more than 1, 2, 3, 4, 5, or 6 modifications; or SEQ ID NOs: 402, 404, and 421, respectively, or an amino acid sequence comprising no more than 1, 2, 3, 4, 5, or 6 modifications. In some embodiments, the HCDR1, HCDR2, and HCDR3 comprise the amino acid sequences of SEQ ID NOs: 402, 404, and 413, respectively. In some embodiments, the HCDR1, HCDR2, and HCDR3 comprise the amino acid sequences of SEQ ID NOs: 402, 405, and 413, respectively. In some embodiments, the HCDR1, HCDR2, and HCDR3 comprise the amino acid sequences of SEQ ID NOs: 402, 406, and 413, respectively. In some embodiments, the HCDR1, HCDR2, and HCDR3 comprise the amino acid sequences of SEQ ID NOs: 402, 404, and 414, respectively. In some embodiments, the HCDR1, HCDR2, and HCDR3 comprise the amino acid sequences of SEQ ID NOs: 402, 407, and 413, respectively. In some embodiments, the HCDR1, HCDR2, and HCDR3 comprise the amino acid sequences of SEQ ID NOs: 402, 404, and 415, respectively. In some embodiments, the HCDR1, HCDR2, and HCDR3 comprise the amino acid sequences of SEQ ID NOs: 402, 408, and 413, respectively. In some embodiments, the HCDR1, HCDR2, and HCDR3 comprise the amino acid sequences of SEQ ID NOs: 402, 404, and 416, respectively. In some embodiments, the HCDR1, HCDR2, and HCDR3 comprise the amino acid sequences of SEQ ID NOs: 402, 404, and 417, respectively. In some embodiments, the HCDR1, HCDR2, and HCDR3 comprise the amino acid sequences of SEQ ID NOs: 402, 409, and 413, respectively. In some embodiments, the HCDR1, HCDR2, and HCDR3 comprise the amino acid sequences of SEQ ID NOs: 402, 404, and 418, respectively. In some embodiments, the HCDR1, HCDR2, and HCDR3 comprise the amino acid sequences of SEQ ID NOs: 402, 404, and 419, respectively. In some embodiments, the HCDR1, HCDR2, and HCDR3 comprise the amino acid sequences of SEQ ID NOs: 402, 404, and 420, respectively. In some embodiments, the HCDR1, HCDR2, and HCDR3 comprise the amino acid sequences of SEQ ID NOs: 402, 404, and 421, respectively.

In some embodiments, the LCDR1, LCDR2, and LCDR3 comprise the amino acid sequences of SEQ ID NOs: 432, 434, and 436, respectively. In some embodiments, the LCDR1, LCDR2, and LCDR3 comprise the amino acid sequences of SEQ ID NOs: 433, 435, and 437, respectively.

In some embodiments, the HCDR1, HCDR2, HCDR3, LCDR1, LCDR2, and LCDR3 comprise the amino acid sequences of SEQ ID NOs: 402, 404, 413, 432, 434, and 436, respectively. In some embodiments, the HCDR1, HCDR2, HCDR3, LCDR1, LCDR2, and LCDR3 comprise the amino acid sequences of SEQ ID NOs: 402, 405, 413, 432, 434, and 436, respectively. In some embodiments, the HCDR1, HCDR2, HCDR3, LCDR1, LCDR2, and LCDR3 comprise the amino acid sequences of SEQ ID NOs: 402, 406, 413, 432, 434, and 436, respectively. In some embodiments, the HCDR1, HCDR2, HCDR3, LCDR1, LCDR2, and LCDR3 comprise the amino acid sequences of SEQ ID NOs: 402, 404, 414, 432, 434, and 436, respectively. In some embodiments, the HCDR1, HCDR2, HCDR3, LCDR1, LCDR2, and LCDR3 comprise the amino acid sequences of SEQ ID NOs: 402, 407, 413, 432, 434, and 436, respectively. In some embodiments, the HCDR1, HCDR2, HCDR3, LCDR1, LCDR2, and LCDR3 comprise the amino acid sequences of SEQ ID NOs: 402, 404, 415, 432, 434, and 436, respectively. In some embodiments, the HCDR1, HCDR2, HCDR3, LCDR1, LCDR2, and LCDR3 comprise the amino acid sequences of SEQ ID NOs: 402, 408, 413, 432, 434, and 436, respectively. In some embodiments, the HCDR1, HCDR2, HCDR3, LCDR1, LCDR2, and LCDR3 comprise the amino acid sequences of SEQ ID NOs: 402, 404, 416, 432, 434, and 436, respectively. In some embodiments, the HCDR1, HCDR2, HCDR3, LCDR1, LCDR2, and LCDR3 comprise the amino acid sequences of SEQ ID NOs: 402, 404, 417, 432, 434, and 436, respectively. In some embodiments, the HCDR1, HCDR2, HCDR3, LCDR1, LCDR2, and LCDR3 comprise the amino acid sequences of SEQ ID NOs: 402, 409, 413, 432, 434, and 436, respectively. In some embodiments, the HCDR1, HCDR2, HCDR3, LCDR1, LCDR2, and LCDR3 comprise the amino acid sequences of SEQ ID NOs: 402, 404, 418, 432, 434, and 436, respectively. In some embodiments, the HCDR1, HCDR2, HCDR3, LCDR1, LCDR2, and LCDR3 comprise the amino acid sequences of SEQ ID NOs: 402, 404, 419, 432, 434, and 436, respectively. In some embodiments, the HCDR1, HCDR2, HCDR3, LCDR1, LCDR2, and LCDR3 comprise the amino acid sequences of SEQ ID NOs: 402, 404, 420, 432, 434, and 436, respectively. In some embodiments, the HCDR1, HCDR2, HCDR3, LCDR1, LCDR2, and LCDR3 comprise the amino acid sequences of SEQ ID NOs: 402, 404, 421, 432, 434, and 436, respectively.

In some embodiments, the VH comprises the amino acid sequence of SEQ ID NO: 323, or an amino acid sequence having at least 90, 92, 94, 96, 98, or 99% identity thereto. In some embodiments, the VH comprises the amino acid sequence of SEQ ID NO: 324, or an amino acid sequence having at least 90, 92, 94, 96, 98, or 99% identity thereto. In some embodiments, the VH comprises the amino acid sequence of SEQ ID NO: 325, or an amino acid sequence having at least 90, 92, 94, 96, 98, or 99% identity thereto. In some embodiments, the VH comprises the amino acid sequence of SEQ ID NO: 326, or an amino acid sequence having at least 90, 92, 94, 96, 98, or 99% identity thereto. In some embodiments, the VH comprises the amino acid sequence of SEQ ID NO: 327, or an amino acid sequence having at least 90, 92, 94, 96, 98, or 99% identity thereto. In some embodiments, the VH comprises the amino acid sequence of SEQ ID NO: 328, or an amino acid sequence having at least 90, 92, 94, 96, 98, or 99% identity thereto. In some embodiments, the VH comprises the amino acid sequence of SEQ ID NO: 329, or an amino acid sequence having at least 90, 92, 94, 96, 98, or 99% identity thereto. In some embodiments, the VH comprises the amino acid sequence of SEQ ID NO: 330, or an amino acid sequence having at least 90, 92, 94, 96, 98, or 99% identity thereto. In some embodiments, the VH comprises the amino acid sequence of SEQ ID NO: 331, or an amino acid sequence having at least 90, 92, 94, 96, 98, or 99% identity thereto. In some embodiments, the VH comprises the amino acid sequence of SEQ ID NO: 332, or an amino acid sequence having at least 90, 92, 94, 96, 98, or 99% identity thereto. In some embodiments, the VH comprises the amino acid sequence of SEQ ID NO: 333, or an amino acid sequence having at least 90, 92, 94, 96, 98, or 99% identity thereto. In some embodiments, the VH comprises the amino acid sequence of SEQ ID NO: 334, or an amino acid sequence having at least 90, 92, 94, 96, 98, or 99% identity thereto. In some embodiments, the VH comprises the amino acid sequence of SEQ ID NO: 335, or an amino acid sequence having at least 90, 92, 94, 96, 98, or 99% identity thereto. In some embodiments, the VH comprises the amino acid sequence of SEQ ID NO: 336, or an amino acid sequence having at least 90, 92, 94, 96, 98, or 99% identity thereto.

In some embodiments, the VL comprises the amino acid sequence of SEQ ID NO: 341, or an amino acid sequence having at least 90, 92, 94, 96, 98, or 99% identity thereto. In some embodiments, the VL comprises the amino acid sequence of SEQ ID NO: 342, or an amino acid sequence having at least 90, 92, 94, 96, 98, or 99% identity thereto.

In some embodiments, the VH and VL comprise the amino acid sequences of SEQ ID NOs: 323 and 341, respectively. In some embodiments, the VH and VL comprise the amino acid sequences of SEQ ID NOs: 324 and 341, respectively. In some embodiments, the VH and VL comprise the amino acid sequences of SEQ ID NOs: 325 and 341, respectively. In some embodiments, the VH and VL comprise the amino acid sequences of SEQ ID NOs: 326 and 341, respectively. In some embodiments, the VH and VL comprise the amino acid sequences of SEQ ID NOs: 327 and 341, respectively. In some embodiments, the VH and VL comprise the amino acid sequences of SEQ ID NOs: 328 and 341, respectively. In some embodiments, the VH and VL comprise the amino acid sequences of SEQ ID NOs: 329 and 341, respectively. In some embodiments, the VH and VL comprise the amino acid sequences of SEQ ID NOs: 330 and 341, respectively. In some embodiments, the VH and VL comprise the amino acid sequences of SEQ ID NOs: 331 and 341, respectively. In some embodiments, the VH and VL comprise the amino acid sequences of SEQ ID NOs: 332 and 341, respectively. In some embodiments, the VH and VL comprise the amino acid sequences of SEQ ID NOs: 333 and 341, respectively. In some embodiments, the VH and VL comprise the amino acid sequences of SEQ ID NOs: 334 and 341, respectively. In some embodiments, the VH and VL comprise the amino acid sequences of SEQ ID NOs: 335 and 341, respectively. In some embodiments, the VH and VL comprise the amino acid sequences of SEQ ID NOs: 336 and 341, respectively.

In one aspect, disclosed herein is an antibody molecule (e.g., an isolated antibody molecule) that binds to CSF1R (e.g., human CSF1R), comprising a heavy chain variable region (VH) comprising a heavy chain complementarity determining region 1 (HCDR1), a HCDR2, and a HCDR3, wherein the HCDR1, HCDR2, and HCDR3 comprise the amino acid sequences of SEQ ID NOs: 403, 478, and 479, respectively, or an amino acid sequence comprising no more than 1, 2, 3, 4, 5, or 6 modifications. In some embodiments, the antibody molecule further comprises a light chain variable region (VL) comprising a light chain complementarity determining region 1 (LCDR1), a LCDR2, and a LCDR3. In some embodiments, the LCDR1, LCDR2, and LCDR3 comprise the amino acid sequences of SEQ ID NOs: 432, 434, and 436, respectively, or an amino acid sequence comprising no more than 1, 2, 3, 4, 5, or 6 modifications. In some embodiments, the LCDR1, LCDR2, and LCDR3 comprise the amino acid sequences of SEQ ID NOs: 433, 435, and 437, respectively, or an amino acid sequence comprising no more than 1, 2, 3, 4, 5, or 6 modifications. In one aspect, disclosed herein is an antibody molecule (e.g., an isolated antibody molecule) that binds to CSF1R (e.g., human CSF1R), comprising a light chain variable region (VL) comprising a light chain complementarity determining region 1 (LCDR1), a LCDR2, and a LCDR3, wherein the LCDR1, LCDR2, and LCDR3 comprise the amino acid sequences of SEQ ID NOs: 432, 434, and 436, respectively, or SEQ ID NOs: 433, 435, and 437, respectively, or an amino acid sequence comprising no more than 1, 2, 3, 4, 5, or 6 modifications.

In some embodiments, the HCDR1, HCDR2, and HCDR3 comprise the amino acid sequences of: SEQ ID NOs: 403, 410, and 422, respectively, or an amino acid sequence comprising no more than 1, 2, 3, 4, 5, or 6 modifications; SEQ ID NOs: 403, 410, and 423, respectively, or an amino acid sequence comprising no more than 1, 2, 3, 4, 5, or 6 modifications; SEQ ID NOs: 403, 411, and 422, respectively, or an amino acid sequence comprising no more than 1, 2, 3, 4, 5, or 6 modifications; or SEQ ID NOs: 403, 412, and 422, respectively, or an amino acid sequence comprising no more than 1, 2, 3, 4, 5, or 6 modifications.

In some embodiments, the LCDR1, LCDR2, and LCDR3 comprise the amino acid sequences of SEQ ID NOs: 432, 434, and 436, respectively. In some embodiments, the LCDR1, LCDR2, and LCDR3 comprise the amino acid sequences of SEQ ID NOs: 433, 435, and 437, respectively.

In some embodiments, the HCDR1, HCDR2, HCDR3, LCDR1, LCDR2, and LCDR3 comprise the amino acid sequences of SEQ ID NOs: 403, 410, 422, 433, 435, and 437, respectively. In some embodiments, the HCDR1, HCDR2, HCDR3, LCDR1, LCDR2, and LCDR3 comprise the amino acid sequences of SEQ ID NOs: 403, 410, 423, 433, 435, and 437, respectively. In some embodiments, the HCDR1, HCDR2, HCDR3, LCDR1, LCDR2, and LCDR3 comprise the amino acid sequences of SEQ ID NOs: 403, 411, 422, 433, 435, and 437, respectively. In some embodiments, the HCDR1, HCDR2, HCDR3, LCDR1, LCDR2, and LCDR3 comprise the amino acid sequences of SEQ ID NOs: 403, 412, 422, 433, 435, and 437, respectively.

In some embodiments, the VH comprises the amino acid sequence of SEQ ID NO: 337, or an amino acid sequence having at least 90, 92, 94, 96, 98, or 99% identity thereto. In some embodiments, the VH comprises the amino acid sequence of SEQ ID NO: 338, or an amino acid sequence having at least 90, 92, 94, 96, 98, or 99% identity thereto. In some embodiments, the VH comprises the amino acid sequence of SEQ ID NO: 339, or an amino acid sequence having at least 90, 92, 94, 96, 98, or 99% identity thereto. In some embodiments, the VH comprises the amino acid sequence of SEQ ID NO: 340, or an amino acid sequence having at least 90, 92, 94, 96, 98, or 99% identity thereto.

In some embodiments, the VL comprises the amino acid sequence of SEQ ID NO: 139, or an amino acid sequence having at least 90, 92, 94, 96, 98, or 99% identity thereto. In some embodiments, the VL comprises the amino acid sequence of SEQ ID NO: 341, or an amino acid sequence having at least 90, 92, 94, 96, 98, or 99% identity thereto. In some embodiments, the VL comprises the amino acid sequence of SEQ ID NO: 342, or an amino acid sequence having at least 90, 92, 94, 96, 98, or 99% identity thereto.

In some embodiments, the VH and VL comprise the amino acid sequences of SEQ ID NOs: 337 and 342, respectively. In some embodiments, the VH and VL comprise the amino acid sequences of SEQ ID NOs: 338 and 342, respectively. In some embodiments, the VH and VL comprise the amino acid sequences of SEQ ID NOs: 339 and 342, respectively. In some embodiments, the VH and VL comprise the amino acid sequences of SEQ ID NOs: 340 and 342, respectively. In some embodiments, the VH and VL comprise the amino acid sequences of SEQ ID NOs: 339 and 139, respectively.

In some embodiment, the antibody molecule binds to human and/or cynomolgus CSF1R, e.g., as measured using an ELISA analysis, e.g., as measured using methods described in Example 22 with respect to FIGS. 29A and 29B. In some embodiment, the antibody molecule binds to cells expressing human CSF1R, e.g., as measured using a flow cytometry analysis, e.g., as measured using methods described in Example 24 with respect to FIG. 31. In some embodiment, the antibody molecule reduces MCP-1 secretion from monocytes in the presence of CSF-1, e.g., as measured using an electrochemiluminescence analysis, e.g., as measured using methods described in Example 23 with respect to FIG. 30. In some embodiment, the antibody molecule reduces an activity of CSF1R.

In one aspect, provided herein is an antibody molecule (e.g., an isolated antibody molecule) that binds to CCR2 (e.g., human CCR2), comprising a heavy chain variable region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3, wherein the HCDR1, HCDR2, HCDR3, LCDR1, LCDR2, and LCDR3 comprise the amino acid sequences of SEQ ID NOs: 446, 447, 448, 454, 455, and 456, respectively, or an amino acid sequence comprising no more than 1, 2, 3, 4, 5, or 6 modifications, and wherein:

(i) the VH does not comprise the amino acid sequence of SEQ ID NO: 480, and/or

(ii) the VL does not comprise the amino acid sequence of SEQ ID NO: 481.

In some embodiments, the HCDR1, HCDR2, HCDR3, LCDR1, LCDR2, and LCDR3 comprise the amino acid sequences of SEQ ID NOs: 446, 447, 448, 454, 455, and 456, respectively. In some embodiments, the VH comprises the amino acid sequence of SEQ ID NO: 343, or an amino acid sequence having at least 90, 92, 94, 96, 98, or 99% identity thereto. In some embodiments, the VH comprises the amino acid sequence of SEQ ID NO: 344, or an amino acid sequence having at least 90, 92, 94, 96, 98, or 99% identity thereto. In some embodiments, the VL comprises the amino acid sequence of SEQ ID NO: 345, or an amino acid sequence having at least 90, 92, 94, 96, 98, or 99% identity thereto. In some embodiments, the VH and VL comprise the amino acid sequences of SEQ ID NOs: 343 and 345, respectively. In some embodiments, the VH and VL comprise the amino acid sequences of SEQ ID NOs: 344 and 345, respectively.

In one aspect, provided herein is an antibody molecule (e.g., an isolated antibody molecule) that binds to PD-L1 (e.g., human PD-L1), comprising a heavy chain variable region (VH) comprising a heavy chain complementarity determining region 1 (HCDR1), a HCDR2, and a HCDR3, wherein the HCDR1, HCDR2, and HCDR3 comprise the amino acid sequences of SEQ ID NOs: 461, 462, and 463, respectively, or an amino acid sequence comprising no more than 1, 2, 3, 4, 5, or 6 modifications. In some embodiments, the antibody molecule further comprises a light chain variable region (VL) comprising a light chain complementarity determining region 1 (LCDR1), a LCDR2, and a LCDR3, wherein the LCDR1, LCDR2, and LCDR3 comprise the amino acid sequences of SEQ ID NOs: 467, 468, and 469, respectively, or an amino acid sequence comprising no more than 1, 2, 3, 4, 5, or 6 modifications. In one aspect, provided herein is an antibody molecule (e.g., an isolated antibody molecule) that binds to PD-L1 (e.g., human PD-L1), comprising a light chain variable region (VL) comprising a light chain complementarity determining region 1 (LCDR1), a LCDR2, and a LCDR3, wherein the LCDR1, LCDR2, and LCDR3 comprise the amino acid sequences of SEQ ID NOs: 467, 468, and 469, respectively, or an amino acid sequence comprising no more than 1, 2, 3, 4, 5, or 6 modifications. In some embodiments, the HCDR1, HCDR2, HCDR3, LCDR1, LCDR2, and LCDR3 comprise the amino acid sequences of SEQ ID NOs: 461, 462, 463, 467, 468, and 469, respectively. In some embodiments, the VH comprises the amino acid sequence of SEQ ID NO: 346, or an amino acid sequence having at least 90, 92, 94, 96, 98, or 99% identity thereto. In some embodiments, the VL comprises the amino acid sequence of SEQ ID NO: 347, or an amino acid sequence having at least 90, 92, 94, 96, 98, or 99% identity thereto. In some embodiments, the VH and VL comprise the amino acid sequences of SEQ ID NOs: 346 and 347, respectively.

In some embodiments of the aforementioned aspects, the antibody molecule comprises a heavy chain constant region chosen from IgG1, IgG2, IgG3, or IgG4, or a fragment thereof. In some embodiments, the antibody molecule comprises a light chain constant region chosen from the light chain constant regions of kappa or lambda, or a fragment thereof.

In one aspect, provided herein is a multispecific, e.g., a bispecific, molecule, comprising an anti-CSF1R binding moiety and an anti-CCR2 binding moiety. In some embodiments, the anti-CSF1R binding moiety comprises an anti-CSF1R antibody molecule disclosed herein. In some embodiments, the anti-CCR2 binding moiety comprises an anti-CCR2 antibody molecule disclosed herein. In some embodiments, the anti-CSF1R binding moiety comprises an anti-CSF1R antibody molecule disclosed herein and the anti-CCR2 binding moiety comprises an anti-CCR2 antibody molecule disclosed herein.

In some embodiments, the anti-CSF1R binding moiety comprises a heavy chain variable region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3, wherein: (i) the HCDR1, HCDR2, and HCDR3 comprise the amino acid sequences of SEQ ID NOs: 402, 474, and 475, respectively; and (ii) the LCDR1, LCDR2, and LCDR3 comprise the amino acid sequences of SEQ ID NOs: 432, 434, and 436, respectively, or SEQ ID NOs: 433, 435, and 437, respectively. In some embodiments, the HCDR1, HCDR2, and HCDR3 comprise the amino acid sequences of SEQ ID NOs: 402, 476, and 477, respectively. In some embodiments, the HCDR1, HCDR2, and HCDR3 comprise the amino acid sequences of: SEQ ID NOs: 402, 404, and 413, respectively; SEQ ID NOs: 402, 405, and 413, respectively; SEQ ID NOs: 402, 406, and 413, respectively; SEQ ID NOs: 402, 404, and 414, respectively; SEQ ID NOs: 402, 407, and 413, respectively; SEQ ID NOs: 402, 404, and 415, respectively; SEQ ID NOs: 402, 408, and 413, respectively; SEQ ID NOs: 402, 404, and 416, respectively; SEQ ID NOs: 402, 404, and 417, respectively; SEQ ID NOs: 402, 409, and 413, respectively; SEQ ID NOs: 402, 404, and 418, respectively; SEQ ID NOs: 402, 404, and 419, respectively; SEQ ID NOs: 402, 404, and 420, respectively; or SEQ ID NOs: 402, 404, and 421, respectively. In some embodiments, the LCDR1, LCDR2, and LCDR3 comprise the amino acid sequences of SEQ ID NOs: 432, 434, and 436, respectively. In some embodiments, the LCDR1, LCDR2, and LCDR3 comprise the amino acid sequences of SEQ ID NOs: 433, 435, and 437, respectively. In some embodiments, the HCDR1, HCDR2, HCDR3, LCDR1, LCDR2, and LCDR3 comprise the amino acid sequences of: SEQ ID NOs: 402, 404, 413, 432, 434, and 436, respectively; SEQ ID NOs: 402, 405, 413, 432, 434, and 436, respectively; SEQ ID NOs: 402, 406, 413, 432, 434, and 436, respectively; SEQ ID NOs: 402, 404, 414, 432, 434, and 436, respectively; SEQ ID NOs: 402, 407, 413, 432, 434, and 436, respectively; SEQ ID NOs: 402, 404, 415, 432, 434, and 436, respectively; SEQ ID NOs: 402, 408, 413, 432, 434, and 436, respectively; SEQ ID NOs: 402, 404, 416, 432, 434, and 436, respectively; SEQ ID NOs: 402, 404, 417, 432, 434, and 436, respectively; SEQ ID NOs: 402, 409, 413, 432, 434, and 436, respectively; SEQ ID NOs: 402, 404, 418, 432, 434, and 436, respectively; SEQ ID NOs: 402, 404, 419, 432, 434, and 436, respectively; SEQ ID NOs: 402, 404, 420, 432, 434, and 436, respectively; or SEQ ID NOs: 402, 404, 421, 432, 434, and 436, respectively. In some embodiments, the VH comprises the amino acid sequence of any of SEQ ID NOs: 323-336, or an amino acid sequence having at least 90, 92, 94, 96, 98, or 99% identity thereto. In some embodiments, the VL comprises the amino acid sequence of SEQ ID NO: 341 or 342, or an amino acid sequence having at least 90, 92, 94, 96, 98, or 99% identity thereto. In some embodiments, the VH and VL comprise the amino acid sequences of: SEQ ID NOs: 323 and 341, respectively; SEQ ID NOs: 324 and 341, respectively; SEQ ID NOs: 325 and 341, respectively; SEQ ID NOs: 326 and 341, respectively; SEQ ID NOs: 327 and 341, respectively; SEQ ID NOs: 328 and 341, respectively; SEQ ID NOs: 329 and 341, respectively; SEQ ID NOs: 330 and 341, respectively; SEQ ID NOs: 331 and 341, respectively; SEQ ID NOs: 332 and 341, respectively; SEQ ID NOs: 333 and 341, respectively; SEQ ID NOs: 334 and 341, respectively; SEQ ID NOs: 335 and 341, respectively; or SEQ ID NOs: 336 and 341, respectively.

In some embodiments, the anti-CSF1R binding moiety comprises a heavy chain variable region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3, wherein: (i) the HCDR1, HCDR2, and HCDR3 comprise the amino acid sequences of SEQ ID NOs: 403, 478, and 479, respectively; and (ii) the LCDR1, LCDR2, and LCDR3 comprise the amino acid sequences of SEQ ID NOs: 432, 434, and 436, respectively, or SEQ ID NOs: 433, 435, and 437, respectively. In some embodiments, the HCDR1, HCDR2, and HCDR3 comprise the amino acid sequences of: SEQ ID NOs: 403, 410, and 422, respectively; SEQ ID NOs: 403, 410, and 423, respectively; SEQ ID NOs: 403, 411, and 422, respectively; or SEQ ID NOs: 403, 412, and 422, respectively. In some embodiments, the LCDR1, LCDR2, and LCDR3 comprise the amino acid sequences of SEQ ID NOs: 432, 434, and 436, respectively. In some embodiments, the LCDR1, LCDR2, and LCDR3 comprise the amino acid sequences of SEQ ID NOs: 433, 435, and 437, respectively. In some embodiments, the HCDR1, HCDR2, HCDR3, LCDR1, LCDR2, and LCDR3 comprise the amino acid sequences of: SEQ ID NOs: 403, 410, 422, 433, 435, and 437, respectively; SEQ ID NOs: 403, 410, 423, 433, 435, and 437, respectively; SEQ ID NOs: 403, 411, 422, 433, 435, and 437, respectively; or SEQ ID NOs: 403, 412, 422, 433, 435, and 437, respectively. In some embodiments, the VH comprises the amino acid sequence of any of SEQ ID NOs: 337-340, or an amino acid sequence having at least 90, 92, 94, 96, 98, or 99% identity thereto. In some embodiments, the VL comprises the amino acid sequence of SEQ ID NO: 341 or 342, or an amino acid sequence having at least 90, 92, 94, 96, 98, or 99% identity thereto. In some embodiments, the VH and VL comprise the amino acid sequences of: SEQ ID NOs: 337 and 342, respectively; SEQ ID NOs: 338 and 342, respectively; SEQ ID NOs: 339 and 342, respectively; or SEQ ID NOs: 340 and 342, respectively.

In some embodiments, the anti-CCR2 binding moiety comprises a heavy chain variable region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3, wherein the HCDR1, HCDR2, HCDR3, LCDR1, LCDR2, and LCDR3 comprise the amino acid sequences of SEQ ID NOs: 446, 447, 448, 454, 455, and 456, respectively, and wherein: (i) the VH does not comprise the amino acid sequence of SEQ ID NO: 480, or (ii) the VL does not comprise the amino acid sequence of SEQ ID NO: 481. In some embodiments, the VH comprises the amino acid sequence of SEQ ID NO: 343 or 344, or an amino acid sequence having at least 90, 92, 94, 96, 98, or 99% identity thereto. In some embodiments, the VL comprises the amino acid sequence of SEQ ID NO: 345, or an amino acid sequence having at least 90, 92, 94, 96, 98, or 99% identity thereto. In some embodiments, the VH and VL comprise the amino acid sequences of: SEQ ID NOs: 343 and 345, respectively, or SEQ ID NOs: 344 and 345, respectively.

In some embodiments, the anti-CSF1R binding moiety and the anti-CCR2 binding moiety are, independently, a full antibody (e.g., an antibody that includes at least one, and preferably two, complete heavy chains, and at least one, and preferably two, complete light chains), or an antigen-binding fragment (e.g., a Fab, F(ab′)₂, Fv, a scFv, a single domain antibody, or a diabody (dAb)). In some embodiments, the anti-CSF1R binding moiety and/or the anti-CCR2 binding moiety comprises a heavy chain constant region chosen from IgG1, IgG2, IgG3, or IgG4, or a fragment thereof. In some embodiments, the anti-CSF1R binding moiety comprises a first heavy chain constant region (e.g., a first Fc region) and the anti-CCR2 binding moiety comprises a second heavy chain constant region (e.g., a second Fc region), wherein the first heavy chain constant region comprises one or more mutations that increase heterodimerization of the first heavy chain constant region and the second heavy chain constant region, relative to a naturally-existing heavy chain constant region, and/or wherein the second heavy chain constant region comprises one or more mutations that increase heterodimerization of the second heavy chain constant region and the first heavy chain constant region, relative to a naturally-existing heavy chain constant region. In some embodiments, the anti-CSF1R binding moiety and/or the anti-CCR2 binding moiety comprises a light chain constant region chosen from the light chain constant regions of kappa or lambda, or a fragment thereof. In some embodiments, the anti-CSF1R binding moiety comprises a kappa light chain constant region, or a fragment thereof, and the anti-CCR2 binding moiety comprises a lambda light chain constant region, or a fragment thereof. In some embodiments, the anti-CSF1R binding moiety comprises a lambda light chain constant region, or a fragment thereof, and the anti-CCR2 binding moiety comprises a kappa light chain constant region, or a fragment thereof. In some embodiments, the anti-CSF1R binding moiety and the anti-CCR2 binding moiety have a common light chain variable region.

In some embodiments, the multispecific molecule binds to CSF1R monovalently, and/or binds to CCR2 monovalently, optionally wherein the multispecific molecule binds to CSF1R monovalently, and binds to CCR2 monovalently. In some embodiments, the multispecific molecule inhibits CSF1R in the presence of CCR2, optionally wherein the multispecific molecule reduces an activity of CSF1R (e.g., CSF1R signaling, e.g., CSF1-induced CSF1R signaling) in a cell, e.g., by at least 40, 50, 60, 70, 80, or 90%, when the cell expresses both CSF1R and CCR2 on the cell surface. In some embodiments, the multispecific molecule does not inhibit or does not substantially inhibit CSF1R in the absence of CCR2, optionally wherein the multispecific molecule does not reduce an activity of CSF1R (e.g., CSF1R signaling, e.g., CSF1-induced CSF1R signaling), or does not reduce an activity of CSF1R by more than 2, 4, 6, 8, 10, or 15%, when the cell expresses CSF1R but not CCR2 on the cell surface. In some embodiments, the multispecific molecule inhibits CCR2 in the presence of CSF1R, optionally wherein the multispecific molecule reduces an activity of CCR2 in a cell, e.g., by at least 40, 50, 60, 70, 80, or 90%, when the cell expresses both CCR2 and CSF1R on the cell surface. In some embodiments, the multispecific molecule does not inhibit or does not substantially inhibit CCR2 in the absence of CSF1R, optionally wherein the multispecific molecule does not reduce an activity of CCR2, or does not reduce an activity of CCR2 by more than 2, 4, 6, 8, 10, or 15%, when the cell expresses CCR2 but not CSF1R on the cell surface.

In some embodiments, the multispecific molecule further comprises a TGF beta inhibitor. In some embodiments, the TGF beta inhibitor inhibits TGF-beta 1, TGF-beta 3, or both TGF-beta 1 and TGF-beta 3, e.g., as measured using the methods described in Example 20 with respect to FIG. 26B. In one embodiment, the TGF beta inhibitor inhibits TGF-beta 1, TGF-beta 2, TGF-beta 3, both TGF-beta 1 and TGF-beta 3, or TGF-beta 1, TGF-beta 2, and TGF-beta 3, e.g., as measured using the methods described in Example 20 with respect to FIG. 26B. In one embodiment, the TGF beta inhibitor comprises a TGF-beta receptor polypeptide (e.g., an extracellular domain of a TGF-beta receptor, or a functional variant thereof). In one embodiment, the TGF-beta inhibitor comprises one, two, or all of: a TGFBR1 polypeptide (e.g., 1, 2, 3, or more of a TGFBR1 polypeptide), a TGFBR2 polypeptide (e.g., 1, 2, 3, or more of a TGFBR2 polypeptide), or a TGFBR3 polypeptide (e.g., 1, 2, 3, or more of a TGFBR3 polypeptide). In one embodiment, the TGF-beta inhibitor comprises a TGFBR1 polypeptide. In one embodiment, the TGF-beta inhibitor comprises an extracellular domain of TGFBR1 or a sequence substantially identical thereto (e.g., a sequence that is at least 80%, 85%, 90%, or 95% identical thereto). In one embodiment, the TGF-beta inhibitor comprises an extracellular domain of SEQ ID NO: 95, 96, 97, 120, 121, or 122, or a sequence substantially identical thereto (e.g., a sequence that is at least 80%, 85%, 90%, or 95% identical thereto). In one embodiment, the TGF-beta inhibitor comprises the amino acid sequence of SEQ ID NO: 104 or 105, or a sequence substantially identical thereto (e.g., a sequence that is at least 80%, 85%, 90%, or 95% identical thereto). In some embodiments, the TGF beta inhibitor comprises a TGFBR2 polypeptide. In some embodiments, the TGF-beta inhibitor comprises an extracellular domain of TGFBR2 or a sequence substantially identical thereto (e.g., a sequence that is at least 80%, 85%, 90%, or 95% identical thereto). In some embodiments, the TGF-beta inhibitor comprises an extracellular domain of SEQ ID NO: 98, 99, 123, or 124, or a sequence substantially identical thereto (e.g., a sequence that is at least 80%, 85%, 90%, or 95% identical thereto). In some embodiments, the TGF-beta inhibitor comprises an amino acid sequence selected from the group consisting of SEQ ID NOs: 100, 101, 102, and 103, or a sequence substantially identical thereto (e.g., a sequence that is at least 80%, 85%, 90%, or 95% identical thereto). In one embodiment, the TGF-beta inhibitor comprises a TGFBR3 polypeptide. In one embodiment, the TGF-beta inhibitor comprises an extracellular domain of TGFBR3 or a sequence substantially identical thereto (e.g., a sequence that is at least 80%, 85%, 90%, or 95% identical thereto). In one embodiment, the TGF-beta inhibitor comprises an extracellular domain of SEQ ID NO: 106, 107, 125, or 126, or a sequence substantially identical thereto (e.g., a sequence that is at least 80%, 85%, 90%, or 95% identical thereto). In one embodiment, the TGF-beta inhibitor comprises the amino acid sequence of SEQ ID NO: 108, or a sequence substantially identical thereto (e.g., a sequence that is at least 80%, 85%, 90%, or 95% identical thereto). In one embodiment, the TGF-beta inhibitor comprises two TGF-beta receptor polypeptides that form a homodimer. In one embodiment, the TGF-beta inhibitor comprises two TGFBR1 polypeptides that form a homodimer. In one embodiment, the TGF-beta inhibitor comprises two TGFBR2 polypeptides that form a homodimer. In one embodiment, the TGF-beta inhibitor comprises two TGFBR3 polypeptides that form a homodimer. In one embodiment, the TGF-beta inhibitor comprises two TGF-beta receptor polypeptides that form a heterodimer. In one embodiment, the TGF-beta inhibitor comprises a TGFBR1 polypeptide and a TGFBR2 polypeptide that form a heterodimer. In one embodiment, the TGF-beta inhibitor comprises a TGFBR1 polypeptide and a TGFBR3 polypeptide that form a heterodimer. In one embodiment, the TGF-beta inhibitor comprises a TGFBR2 polypeptide and a TGFBR3 polypeptide that form a heterodimer.

In one embodiment, the TGF-beta inhibitor comprises a first TGF-beta receptor polypeptide and a second TGF-beta receptor polypeptide. In one embodiment, the multispecific molecule comprises a first Fc region (e.g., a first CH1-Fc region) and a second Fc region (e.g., a second CH1-Fc region), optionally wherein: (i) the first TGF-beta receptor polypeptide is linked, e.g., via a linker, to the first Fc region (e.g., a first CH1-Fc region), e.g., the C-terminus of the first Fc region (e.g., a first CH1-Fc region), and (ii) the second TGF-beta receptor polypeptide is linked, e.g., via a linker, to the second Fc region (e.g., a second CH1-Fc region), e.g., the C-terminus of the second Fc region (e.g., a second CH1-Fc region). In one embodiment, the first TGF-beta receptor polypeptide and the second TGF-beta receptor polypeptide form a homodimer or heterodimer, e.g., a homodimer. In one embodiment, the first or second TGF-beta receptor polypeptide comprises an extracellular domain of TGFBR1, TGFBR2, or TGFBR3, e.g., an extracellular domain of TGFBR2. In one embodiment, the multispecific molecule has the configuration of FIG. 35A or 35B. In one embodiment, the multispecific molecule comprises the amino acid sequence of SEQ ID NO: 192 (or a sequence substantially identical thereto, e.g., a sequence that is at least 80%, 85%, 90%, or 95% identical thereto) and the amino acid sequence of SEQ ID NO: 193 (or a sequence substantially identical thereto, e.g., a sequence that is at least 80%, 85%, 90%, or 95% identical thereto). In one embodiment, the multispecific molecule comprises the amino acid sequence of SEQ ID NO: 192 (or a sequence substantially identical thereto, e.g., a sequence that is at least 80%, 85%, 90%, or 95% identical thereto) and the amino acid sequence of SEQ ID NO: 195 (or a sequence substantially identical thereto, e.g., a sequence that is at least 80%, 85%, 90%, or 95% identical thereto). In one embodiment, the multispecific molecule comprises the amino acid sequence of SEQ ID NO: 194 (or a sequence substantially identical thereto, e.g., a sequence that is at least 80%, 85%, 90%, or 95% identical thereto) and the amino acid sequence of SEQ ID NO: 193 (or a sequence substantially identical thereto, e.g., a sequence that is at least 80%, 85%, 90%, or 95% identical thereto). In one embodiment, the multispecific molecule comprises the amino acid sequence of SEQ ID NO: 194 (or a sequence substantially identical thereto, e.g., a sequence that is at least 80%, 85%, 90%, or 95% identical thereto) and the amino acid sequence of SEQ ID NO: 195 (or a sequence substantially identical thereto, e.g., a sequence that is at least 80%, 85%, 90%, or 95% identical thereto).

In one embodiment, the TGF-beta inhibitor comprises a first TGF-beta receptor polypeptide and a second TGF-beta receptor polypeptide. In one embodiment, the multispecific molecule comprises a heavy chain constant region 1 (CH1) and a light chain constant region (CL), optionally wherein: (i) the first TGF-beta receptor polypeptide is linked, e.g., via a linker, to the CH1, e.g., the N-terminus of the CH1, and (ii) the second TGF-beta receptor polypeptide is linked, e.g., via a linker, to the CL, e.g., the N-terminus of the CL. In one embodiment, the first TGF-beta receptor polypeptide and the second TGF-beta receptor polypeptide form a homodimer or heterodimer, e.g., a homodimer. In one embodiment, the first or second TGF-beta receptor polypeptide comprises an extracellular domain of TGFBR1, TGFBR2, or TGFBR3, e.g., an extracellular domain of TGFBR2. In one embodiment, the multispecific molecule has the configuration of FIG. 35C or 35D. In one embodiment, the multispecific molecule comprises the amino acid sequence of SEQ ID NO: 196 (or a sequence substantially identical thereto, e.g., a sequence that is at least 80%, 85%, 90%, or 95% identical thereto) and the amino acid sequence of SEQ ID NO: 198 (or a sequence substantially identical thereto, e.g., a sequence that is at least 80%, 85%, 90%, or 95% identical thereto). In one embodiment, the multispecific molecule comprises the amino acid sequence of SEQ ID NO: 196 (or a sequence substantially identical thereto, e.g., a sequence that is at least 80%, 85%, 90%, or 95% identical thereto) and the amino acid sequence of SEQ ID NO: 199 (or a sequence substantially identical thereto, e.g., a sequence that is at least 80%, 85%, 90%, or 95% identical thereto). In one embodiment, the multispecific molecule comprises the amino acid sequence of SEQ ID NO: 197 (or a sequence substantially identical thereto, e.g., a sequence that is at least 80%, 85%, 90%, or 95% identical thereto) and the amino acid sequence of SEQ ID NO: 198 (or a sequence substantially identical thereto, e.g., a sequence that is at least 80%, 85%, 90%, or 95% identical thereto). In one embodiment, the multispecific molecule comprises the amino acid sequence of SEQ ID NO: 197 (or a sequence substantially identical thereto, e.g., a sequence that is at least 80%, 85%, 90%, or 95% identical thereto) and the amino acid sequence of SEQ ID NO: 199 (or a sequence substantially identical thereto, e.g., a sequence that is at least 80%, 85%, 90%, or 95% identical thereto).

In some embodiments, the multispecific molecule further comprises an anti-PD-L1 binding moiety. In some embodiments, the anti-PD-L1 binding moiety comprises an anti-PD-L1 antibody molecule disclosed herein. In some embodiments, the anti-PD-L1 binding moiety inhibits PD-L1. In some embodiments, the anti-PDL1 binding moiety comprises a heavy chain variable region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3, wherein the HCDR1, HCDR2, HCDR3, LCDR1, LCDR2, and LCDR3 comprise the amino acid sequences of SEQ ID NOs: 461, 462, 463, 467, 468, and 469, respectively. In some embodiments, the VH comprises the amino acid sequence of SEQ ID NO: 346, or an amino acid sequence having at least 90, 92, 94, 96, 98, or 99% identity thereto. In some embodiments, the VL comprises the amino acid sequence of SEQ ID NO: 347, or an amino acid sequence having at least 90, 92, 94, 96, 98, or 99% identity thereto. In some embodiments, the VH and VL comprise the amino acid sequences of SEQ ID NOs: 346 and 347, respectively.

In some embodiments, the multispecific molecule further comprises an IL-2 molecule. In some embodiments, the IL-2 molecule is an IL-2 molecule disclosed herein, e.g., an IL-2 molecule comprising the amino acid sequence of SEQ ID NO: 237 or 238, or a sequence substantially identical thereto (e.g., a sequence that is at least 80%, 85%, 90%, or 95% identical thereto).

In some embodiments, the multispecific molecule comprises one or more CDRs (e.g., 1, 2, or 3 CDRs), one or more (e.g., 1 or 2) heavy chain variable regions, one or more (e.g., 1 or 2) light chain variable regions, a heavy chain, or a light chain of an amino acid sequence disclosed in Table 25, or an amino acid sequence having at least 90, 92, 94, 96, 98, or 99% identity thereto. In some embodiments, the multispecific molecule comprises one or more CDRs (e.g., 1, 2, 3, 4, 5, 6, 7, 8, 9, 10, 11, or 12 CDRs), one or more (e.g., 1 or 2) heavy chain variable regions, one or more (e.g., 1 or 2) light chain variable regions, one or more (e.g., 1 or 2) heavy chains, and/or one or more (e.g., 1 or 2) light chains of a multispecific molecule disclosed in Table 34 or Table 28. In some embodiments, the multispecific molecule comprises a multispecific molecule disclosed in Table 34 or Table 28. In some embodiments, the multispecific molecule is or comprises any of molecules 10-86 disclosed in Table 34. In some embodiments, the multispecific molecule is or comprises BIM0648 or BIM0652 disclosed in Table 34. In some embodiments, the multispecific molecule is or comprises any of molecules 10-86 disclosed in Table 28. In some embodiments, the multispecific molecule is or comprises any of molecules BIM0204, BIM0205, BIM0206, BIM0207, BIM0208, BIM0209, BIM0210, BIM0211, BIM0542, BIM0543, BIM0544, BIM0545, BIM0546, BIM0547, BIM0548, BIM0549, BIM0550, BIM0551, BIM0552, BIM0553, BIM0554, BIM0555, BIM0556, BIM0566, and BIM0567 disclosed in Table 28.

In some embodiments, the multispecific molecule has a configuration shown in any of FIGS. 12A-12J.

In one aspect, disclosed herein is a multispecific molecule comprising: (i) an anti-CSF1R binding moiety (e.g., an anti-CSF1R antibody molecule); (ii) an anti-CCR2 binding moiety (e.g., an anti-CCR2 antibody molecule); and (iii) a TGF-beta inhibitor. In some embodiments, the multispecific molecule has one or more (e.g., 1, 2, 3, 4, 5, 6, 7, 8, 9, 10, 11, 12, 13, 14, 15, 16, or more) of the following properties:

(i) the multispecific molecule preferentially binds to a CSF1R-positive, CCR2-positive cell relative to a CSF1R-positive, CCR2-negative cell, or a CSF1R-negative, CCR2-positive cell, e.g., the binding of the multispecific molecule to the CSF1R-positive, CCR2-positive cell is at least 2, 4, 6, 8, 10, 15, 20, or 25-fold stronger than the binding of the multispecific molecule to the CSF1R-positive, CCR2-negative cell, or the CSF1R-negative, CCR2-positive cell, e.g., as measured using a flow cytometry analysis, e.g., as measured using methods described in Example 2 with respect to FIG. 1;
(ii) the multispecific molecule preferentially binds to a CSF1R-positive, CCR2-positive cell relative to a CSF1R-positive, CCR2-negative cell, e.g., the EC50 of the multispecific molecule for binding to a CSF1R-positive, CCR2-positive cell is no more than 60, 50, 40, 30, 20, or 10% of the EC50 of the multispecific molecule for binding to a CSF1R-positive, CCR2-negative cell, e.g., as measured using a flow cytometry analysis, e.g., as measured using methods described in Example 2 with respect to FIG. 1;

(iii) the multispecific molecule preferentially binds to a CSF1R-positive, CCR2-positive cell relative to a CSF1R-negative, CCR2-positive cell, e.g., the EC50 of the multispecific molecule for binding to a CSF1R-positive, CCR2-positive cell is no more than 50, 40, 30, 20, 10, or 5% of the EC50 of the multispecific molecule for binding to a CSF1R-negative, CCR2-positive cell, e.g., as measured using a flow cytometry analysis, e.g., as measured using methods described in Example 2 with respect to FIG. 1;

(iv) the multispecific molecule preferentially binds to tumor-associated macrophages (TAMs) or myeloid derived suppressor cells (MDSCs) relative to T cells, NK cells, neutrophils, tissue-resident macrophages (e.g., Kupffer cells), or Langerhans cells, e.g., the binding of the multispecific molecule to TAMs or MDSCs is at least 2, 4, 6, 8, 10, 15, 20, or 25-fold stronger than the binding of the multispecific molecule to T cells, NK cells, neutrophils, tissue-resident macrophages (e.g., Kupffer cells), or Langerhans cells, e.g., as measured using a flow cytometry analysis, e.g., as measured using methods described in Example 6 with respect to FIG. 5;

(v) the multispecific molecule inhibits monocyte migration, e.g., monocyte chemoattractant protein 1 (MCP1)-induced monocyte migration, e.g., reduces MCP1-induced monocyte migration by at least 40, 50, 60, or 70%, e.g., as measured using a transwell plate migration assay, e.g., as measured using methods described in Example 3 with respect to FIG. 2;

(vi) the multispecific molecule inhibits the proliferation of macrophages, e.g., bone marrow-derived macrophages, e.g., CSF-1-induced proliferation of bone marrow-derived macrophages, e.g., reduces CSF-1-induced proliferation of bone marrow-derived macrophages by at least 50, 60, 70, or 80%, e.g., as measured using a cell proliferation MTT assay, e.g., as measured using methods described in Example 4 with respect to FIG. 3B;

(vii) the multispecific molecule does not inhibit or does not substantially inhibit the differentiation of monocytes, e.g., bone marrow-derived monocytes, e.g., CSF-1-induced differentiation of bone marrow-derived monocytes, e.g., does not reduce CSF-1-induced differentiation of bone marrow-derived monocytes by more than 2, 4, 6, 8, or 10%, e.g., as measured using a flow cytometry analysis, e.g., as measured using methods described in Example 5 with respect to FIG. 4;

(viii) the multispecific molecule depletes suppressive myeloid cells, e.g., TAMs or MDSCs, e.g., reduces the number of suppressive myeloid cells, e.g., TAMs or MDSCs, by at least 80, 85, 90, 95, 99, or 99.5%, in vivo, e.g., as measured using a flow cytometry analysis, e.g., as measured using methods described in Example 7 with respect to FIG. 6;

(ix) the multispecific molecule does not deplete or does not substantially deplete tissue-resident macrophages, e.g., Kupffer cells, e.g., does not reduce the number of tissue-resident macrophages, e.g., Kupffer cells, by more than 4, 6, 8, 10, or 15%, in vivo, e.g., as measured using an immunohistochemistry analysis, e.g., as measured using methods described in Example 8 with respect to FIGS. 7B and 7D;

(x) the multispecific molecule increases CD86 or MHC class II expression on TAMs, e.g., as measured using a flow cytometry analysis or an immunohistochemistry analysis, e.g., as measured using methods described with respect to FIG. 21;

(xi) the multispecific molecule does not inhibit or does not substantially inhibit CSF-1 dependent cell survival of CSF1R-positive, CCR2-negative cells, e.g., does not reduce CSF-1 dependent cell survival of CSF1R-positive, CCR2-negative cells by more than 5, 10, or 15%, e.g., as measured using a cell viability MTT assay, e.g., as measured using methods described in Example 9 with respect to FIG. 8A;

(xii) the multispecific molecule increases CD8+ T cell tumor infiltration in vivo, e.g., increases % CD8+ T cells in CD3+ T cells in tumor by at least 1.5, 2, or 2.5-fold, e.g., as measured using a flow cytometry analysis, e.g., as measured using methods described in Example 10 with respect to FIG. 9;

(xiii) the multispecific molecule reduces Treg frequency in tumor in vivo, e.g., reduces Treg frequency in tumor by at least 15, 20, 25, or 30%, e.g., as measured using a flow cytometry analysis, e.g., as measured using methods described in Example 11 with respect to FIG. 10A; (xiv) the multispecific molecule increases the CD8+ T cell/Treg ratio in tumor in vivo, e.g., increases the CD8+ T cell/Treg ratio in tumor by at least 2.5, 3, 3.5, 4, or 4.5-fold, e.g., as measured using a flow cytometry analysis, e.g., as measured using methods described in Example 11 with respect to FIG. 10B;

(xv) the multispecific molecule reduces tumor growth, increases survival of a tumor-bearing animal, and/or enhances anti-tumor immune memory, e.g., as measured using methods described in Example 12 with respect to FIGS. 11A and 111B, or Example 19 with respect to FIG. 23;

(xvi) the multispecific molecule preferentially binds to classical monocytes relative to intermediate monocytes or non-classical monocytes, e.g., as measured using methods described in Example 28 with respect to FIGS. 39B and 39D; or

(xvii) the multispecific molecule reduces the activity of TGFβ, e.g., by at least 30, 40, 50, 60, 70, 80, or 90%, e.g., as measured using methods described in Example 28 with respect to FIGS. 40A-40F.

In some embodiments, the anti-CSF1R binding moiety comprises a heavy chain variable region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3, wherein the HCDR1, HCDR2, HCDR3, LCDR1, LCDR2, and LCDR3 comprise the amino acid sequences of SEQ ID NOs: 402, 474, 475, 432, 434, and 436, respectively. In some embodiments, the HCDR1, HCDR2, and HCDR3 comprise the amino acid sequences of SEQ ID NOs: 402, 476, and 477, respectively. In some embodiments, the HCDR1, HCDR2, HCDR3, LCDR1, LCDR2, and LCDR3 comprise the amino acid sequences of SEQ ID NOs: 402, 405, 413, 432, 434, and 436, respectively. In some embodiments, the HCDR1, HCDR2, HCDR3, LCDR1, LCDR2, and LCDR3 comprise the amino acid sequences of SEQ ID NOs: 402, 404, 413, 432, 434, and 436, respectively. In some embodiments, the HCDR1, HCDR2, HCDR3, LCDR1, LCDR2, and LCDR3 comprise the amino acid sequences of SEQ ID NOs: 402, 406, 413, 432, 434, and 436, respectively. In some embodiments, the HCDR1, HCDR2, HCDR3, LCDR1, LCDR2, and LCDR3 comprise the amino acid sequences of SEQ ID NOs: 402, 404, 414, 432, 434, and 436, respectively. In some embodiments, the HCDR1, HCDR2, HCDR3, LCDR1, LCDR2, and LCDR3 comprise the amino acid sequences of SEQ ID NOs: 402, 407, 413, 432, 434, and 436, respectively. In some embodiments, the HCDR1, HCDR2, HCDR3, LCDR1, LCDR2, and LCDR3 comprise the amino acid sequences of SEQ ID NOs: 402, 404, 415, 432, 434, and 436, respectively. In some embodiments, the HCDR1, HCDR2, HCDR3, LCDR1, LCDR2, and LCDR3 comprise the amino acid sequences of SEQ ID NOs: 402, 408, 413, 432, 434, and 436, respectively. In some embodiments, the HCDR1, HCDR2, HCDR3, LCDR1, LCDR2, and LCDR3 comprise the amino acid sequences of SEQ ID NOs: 402, 404, 416, 432, 434, and 436, respectively. In some embodiments, the HCDR1, HCDR2, HCDR3, LCDR1, LCDR2, and LCDR3 comprise the amino acid sequences of SEQ ID NOs: 402, 404, 417, 432, 434, and 436, respectively. In some embodiments, the HCDR1, HCDR2, HCDR3, LCDR1, LCDR2, and LCDR3 comprise the amino acid sequences of SEQ ID NOs: 402, 409, 413, 432, 434, and 436, respectively. In some embodiments, the HCDR1, HCDR2, HCDR3, LCDR1, LCDR2, and LCDR3 comprise the amino acid sequences of SEQ ID NOs: 402, 404, 418, 432, 434, and 436, respectively. In some embodiments, the HCDR1, HCDR2, HCDR3, LCDR1, LCDR2, and LCDR3 comprise the amino acid sequences of SEQ ID NOs: 402, 404, 419, 432, 434, and 436, respectively. In some embodiments, the HCDR1, HCDR2, HCDR3, LCDR1, LCDR2, and LCDR3 comprise the amino acid sequences of SEQ ID NOs: 402, 404, 420, 432, 434, and 436, respectively. In some embodiments, the HCDR1, HCDR2, HCDR3, LCDR1, LCDR2, and LCDR3 comprise the amino acid sequences of SEQ ID NOs: 402, 404, 421, 432, 434, and 436, respectively.

In some embodiments, the VH comprises the amino acid sequence of any of SEQ ID NOs: 323-336, or an amino acid sequence having at least 80, 85, 90, or 95% identity thereto. In some embodiments, the VH comprises the amino acid sequence of SEQ ID NO: 324, or an amino acid sequence having at least 80, 85, 90, or 95% identity thereto. In some embodiments, the VH comprises an amino acid sequence encoded by the nucleotide sequence of SEQ ID NO: 129, or a nucleotide sequence having at least 80, 85, 90, or 95% identity thereto. In some embodiments, the VL comprises the amino acid sequence of SEQ ID NO: 341, or an amino acid sequence having at least 80, 85, 90, or 95% identity thereto. In some embodiments, the VL comprises an amino acid sequence encoded by the nucleotide sequence of SEQ ID NO: 130, or a nucleotide sequence having at least 80, 85, 90, or 95% identity thereto. In some embodiments, the VH and VL comprise the amino acid sequences of: SEQ ID NOs: 324 and 341, respectively, SEQ ID NOs: 323 and 341, respectively, SEQ ID NOs: 325 and 341, respectively, SEQ ID NOs: 326 and 341, respectively, SEQ ID NOs: 327 and 341, respectively, SEQ ID NOs: 328 and 341, respectively, SEQ ID NOs: 329 and 341, respectively, SEQ ID NOs: 330 and 341, respectively, SEQ ID NOs: 331 and 341, respectively, SEQ ID NOs: 332 and 341, respectively, SEQ ID NOs: 333 and 341, respectively, SEQ ID NOs: 334 and 341, respectively, SEQ ID NOs: 335 and 341, respectively, or SEQ ID NOs: 336 and 341, respectively.

In some embodiments, the multispecific molecule comprises the amino acid sequence of SEQ ID NO: 127, or an amino acid sequence having at least 80, 85, 90, or 95% identity thereto. In some embodiments, the multispecific molecule comprises an amino acid sequence encoded by the nucleotide sequence of SEQ ID NO: 128, or a nucleotide sequence having at least 80, 85, 90, or 95% identity thereto.

In some embodiments, the anti-CSF1R binding moiety comprises a heavy chain variable region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3, wherein the HCDR1, HCDR2, HCDR3, LCDR1, LCDR2, and LCDR3 comprise the amino acid sequences of SEQ ID NOs: 403, 478, 479, 433, 435, and 437, respectively. In some embodiments, the HCDR1, HCDR2, HCDR3, LCDR1, LCDR2, and LCDR3 comprise the amino acid sequences of SEQ ID NOs: 403, 411, 422, 433, 435, and 437, respectively. In some embodiments, the HCDR1, HCDR2, HCDR3, LCDR1, LCDR2, and LCDR3 comprise the amino acid sequences of SEQ ID NOs: 403, 410, 422, 433, 435, and 437, respectively. In some embodiments, the HCDR1, HCDR2, HCDR3, LCDR1, LCDR2, and LCDR3 comprise the amino acid sequences of SEQ ID NOs: 403, 410, 423, 433, 435, and 437, respectively. In some embodiments, the HCDR1, HCDR2, HCDR3, LCDR1, LCDR2, and LCDR3 comprise the amino acid sequences of SEQ ID NOs: 403, 412, 422, 433, 435, and 437, respectively.

In some embodiments, the VH comprises the amino acid sequence of any of SEQ ID NOs: 337-340, or an amino acid sequence having at least 90, 92, 94, 96, 98, or 99% identity thereto, optionally wherein the VH comprises the amino acid sequence of SEQ ID NO: 339, or an amino acid sequence having at least 80, 85, 90, or 95% identity thereto. In some embodiments, the VH comprises an amino acid sequence encoded by the nucleotide sequence of SEQ ID NO: 138, or a nucleotide sequence having at least 80, 85, 90, or 95% identity thereto. In some embodiments, the VL comprises the amino acid sequence of SEQ ID NO: 139 or 342, or an amino acid sequence having at least 90, 92, 94, 96, 98, or 99% identity thereto, optionally wherein the VL comprises the amino acid sequence of SEQ ID NO: 139, or an amino acid sequence having at least 80, 85, 90, or 95% identity thereto. In some embodiments, the VL comprises an amino acid sequence encoded by the nucleotide sequence of SEQ ID NO: 140, or a nucleotide sequence having at least 80, 85, 90, or 95% identity thereto. In some embodiments, the VH and VL comprise the amino acid sequences of: SEQ ID NOs: 339 and 139, respectively, SEQ ID NOs: 337 and 342, respectively, SEQ ID NOs: 338 and 342, respectively, SEQ ID NOs: 339 and 342, respectively, or SEQ ID NOs: 340 and 342, respectively.

In some embodiments, the multispecific molecule comprises the amino acid sequence of SEQ ID NO: 136, or an amino acid sequence having at least 80, 85, 90, or 95% identity thereto. In some embodiments, the multispecific molecule comprises an amino acid sequence encoded by the nucleotide sequence of SEQ ID NO: 137, or a nucleotide sequence having at least 80, 85, 90, or 95% identity thereto.

In some embodiments, the anti-CCR2 binding moiety comprises a heavy chain variable region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3, wherein the HCDR1, HCDR2, HCDR3, LCDR1, LCDR2, and LCDR3 comprise the amino acid sequences of SEQ ID NOs: 446, 447, 448, 454, 455, and 456, respectively. In some embodiments, the VH does not comprise the amino acid sequence of SEQ ID NO: 480. In some embodiments, the VL does not comprise the amino acid sequence of SEQ ID NO: 481. In some embodiments, the VH does not comprise the amino acid sequence of SEQ ID NO: 480 and the VL does not comprise the amino acid sequence of SEQ ID NO: 481.

In some embodiments, the VH comprises the amino acid sequence of SEQ ID NO: 343 or 344, or an amino acid sequence having at least 90, 92, 94, 96, 98, or 99% identity thereto, optionally wherein the VH comprises the amino acid sequence of SEQ ID NO: 343, or an amino acid sequence having at least 90, 92, 94, 96, 98, or 99% identity thereto. In some embodiments, the VH comprises an amino acid sequence encoded by the nucleotide sequence of SEQ ID NO: 133, or a nucleotide sequence having at least 80, 85, 90, or 95% identity thereto. In some embodiments, the VL comprises the amino acid sequence of SEQ ID NO: 345, or an amino acid sequence having at least 90, 92, 94, 96, 98, or 99% identity thereto. In some embodiments, the VL comprises an amino acid sequence encoded by the nucleotide sequence of SEQ ID NO: 272 or a nucleotide sequence having at least 80, 85, 90, or 95% identity thereto. In some embodiments, the VH and VL comprise the amino acid sequences of: SEQ ID NOs: 343 and 345, respectively, or SEQ ID NOs: 344 and 345, respectively.

In some embodiments, the multispecific molecule comprises the amino acid sequence of the amino acid sequence of SEQ ID NO: 131, or an amino acid sequence having at least 80, 85, 90, or 95% identity thereto. In some embodiments, the multispecific molecule comprises an amino acid sequence encoded by the nucleotide sequence of SEQ ID NO: 132, or a nucleotide sequence having at least 80, 85, 90, or 95% identity thereto. In some embodiments, the multispecific molecule comprises the amino acid sequence of SEQ ID NO: 373, or an amino acid sequence having at least 80, 85, 90, or 95% identity thereto. In some embodiments, the multispecific molecule comprises an amino acid sequence encoded by the nucleotide sequence of SEQ ID NO: 134, or a nucleotide sequence having at least 80, 85, 90, or 95% identity thereto.

In some embodiments, the multispecific molecule comprises the amino acid sequence of SEQ ID NO: 127, the amino acid sequence of SEQ ID NO: 131, the amino acid sequence of SEQ ID NO: 373. In some embodiments, the multispecific molecule comprises the amino acid sequence of SEQ ID NO: 136, the amino acid sequence of SEQ ID NO: 131, the amino acid sequence of SEQ ID NO: 373.

In some embodiments, the anti-CSF1R antibody molecule binds to CSF1R monovalently. In some embodiments, the anti-CCR2 antibody molecule binds to CCR2 monovalently. In some embodiments, the multispecific molecule binds to CSF1R monovalently, and binds to CCR2 monovalently. In some embodiments, the multispecific molecule inhibits CSF1R in the presence of CCR2, optionally wherein the multispecific molecule reduces an activity of CSF1R (e.g., CSF1R signaling, e.g., CSF1-induced CSF1R signaling) in a cell, e.g., by at least 40, 50, 60, 70, 80, or 90%, when the cell expresses both CSF1R and CCR2 on the cell surface. In some embodiments, the multispecific molecule does not inhibit or does not substantially inhibit CSF1R in the absence of CCR2, optionally wherein the multispecific molecule does not reduce an activity of CSF1R (e.g., CSF1R signaling, e.g., CSF1-induced CSF1R signaling), or does not reduce an activity of CSF1R by more than 2, 4, 6, 8, 10, or 15%, when the cell expresses CSF1R but not CCR2 on the cell surface.

In some embodiments, the multispecific molecule inhibits CCR2 in the presence of CSF1R, optionally wherein the multispecific molecule reduces an activity of CCR2 in a cell, e.g., by at least 40, 50, 60, 70, 80, or 90%, when the cell expresses both CCR2 and CSF1R on the cell surface. In some embodiments, the multispecific molecule does not inhibit or does not substantially inhibit CCR2 in the absence of CSF1R, optionally wherein the multispecific molecule does not reduce an activity of CCR2, or does not reduce an activity of CCR2 by more than 2, 4, 6, 8, 10, or 15%, when the cell expresses CCR2 but not CSF1R on the cell surface.

In some embodiments, the TGF beta inhibitor inhibits TGF-beta 1, TGF-beta 2, TGF-beta 3, both TGF-beta 1 and TGF-beta 3, or TGF-beta 1, TGF-beta 2, and TGF-beta 3, e.g., as measured using the methods described in Example 20 with respect to FIG. 26B. In some embodiments, the TGF beta inhibitor comprises a TGF-beta receptor polypeptide (e.g., an extracellular domain of a TGF-beta receptor, or a functional variant thereof). In some embodiments, the TGF-beta inhibitor comprises one, two, or all of: a TGFBR1 polypeptide (e.g., 1, 2, 3, or more of a TGFBR1 polypeptide), a TGFBR2 polypeptide (e.g., 1, 2, 3, or more of a TGFBR2 polypeptide), or a TGFBR3 polypeptide (e.g., 1, 2, 3, or more of a TGFBR3 polypeptide). In some embodiments, the TGF-beta inhibitor comprises a TGFBR1 polypeptide. In some embodiments, the TGF-beta inhibitor comprises: (i) an extracellular domain of TGFBR1 or a sequence substantially identical thereto (e.g., a sequence that is at least 80%, 85%, 90%, or 95% identical thereto), (ii) an extracellular domain of SEQ ID NO: 95, 96, 97, 120, 121, or 122, or a sequence substantially identical thereto (e.g., a sequence that is at least 80%, 85%, 90%, or 95% identical thereto), or (iii) the amino acid sequence of SEQ ID NO: 104 or 105, or a sequence substantially identical thereto (e.g., a sequence that is at least 80%, 85%, 90%, or 95% identical thereto).

In some embodiments, the TGF-beta inhibitor comprises a TGFBR2 polypeptide. In some embodiments, the TGF-beta inhibitor comprises: (i) an extracellular domain of TGFBR2 or a sequence substantially identical thereto (e.g., a sequence that is at least 80%, 85%, 90%, or 95% identical thereto), (ii) an extracellular domain of SEQ ID NO: 98, 99, 123, or 124, or a sequence substantially identical thereto (e.g., a sequence that is at least 80%, 85%, 90%, or 95% identical thereto), or (iii) an amino acid sequence selected from the group consisting of SEQ ID NOs: 100, 101, 102, and 103, or a sequence substantially identical thereto (e.g., a sequence that is at least 80%, 85%, 90%, or 95% identical thereto).

In some embodiments, the TGF-beta inhibitor comprises a TGFBR3 polypeptide. In some embodiments, the TGF-beta inhibitor comprises: (i) an extracellular domain of TGFBR3 or a sequence substantially identical thereto (e.g., a sequence that is at least 80%, 85%, 90%, or 95% identical thereto), (ii) an extracellular domain of SEQ ID NO: 106, 107, 125, or 126, or a sequence substantially identical thereto (e.g., a sequence that is at least 80%, 85%, 90%, or 95% identical thereto), or (iii) the amino acid sequence of SEQ ID NO: 108, or a sequence substantially identical thereto (e.g., a sequence that is at least 80%, 85%, 90%, or 95% identical thereto).

In some embodiments, the TGF-beta inhibitor comprises two TGF-beta receptor polypeptides that form a homodimer, optionally wherein the TGF-beta inhibitor comprises: (i) two TGFBR1 polypeptides that form a homodimer, (ii) two TGFBR2 polypeptides that form a homodimer, or (iii) two TGFBR3 polypeptides that form a homodimer.

In some embodiments, the TGF-beta inhibitor comprises two TGF-beta receptor polypeptides that form a heterodimer. In some embodiments, the TGF-beta inhibitor comprises: (i) a TGFBR1 polypeptide and a TGFBR2 polypeptide that form a heterodimer, (ii) a TGFBR1 polypeptide and a TGFBR3 polypeptide that form a heterodimer, or (iii) a TGFBR2 polypeptide and a TGFBR3 polypeptide that form a heterodimer.

In some embodiments, the TGF-beta inhibitor comprises a first TGF-beta receptor polypeptide and a second TGF-beta receptor polypeptide.

In some embodiments, the multispecific molecule comprises a first Fc region (e.g., a first CH1-Fc region) and a second Fc region (e.g., a second CH1-Fc region). In some embodiments, (i) the first TGF-beta receptor polypeptide is linked, e.g., via a linker, to the first Fc region (e.g., a first CH1-Fc region), e.g., the C-terminus of the first Fc region (e.g., a first CH1-Fc region), and (ii) the second TGF-beta receptor polypeptide is linked, e.g., via a linker, to the second Fc region (e.g., a second CH1-Fc region), e.g., the C-terminus of the second Fc region (e.g., a second CH1-Fc region). In some embodiments, the first TGF-beta receptor polypeptide and the second TGF-beta receptor polypeptide form a homodimer or heterodimer, e.g., a homodimer. In some embodiments, the first or second TGF-beta receptor polypeptide comprises an extracellular domain of TGFBR1, TGFBR2, or TGFBR3, e.g., an extracellular domain of TGFBR2. In some embodiments, the multispecific molecule has the configuration of FIG. 35A or 35B.

In some embodiments, the multispecific molecule comprises the amino acid sequence of SEQ ID NO: 192 and the amino acid sequence of SEQ ID NO: 193. In some embodiments, the multispecific molecule comprises the amino acid sequence of SEQ ID NO: 192 and the amino acid sequence of SEQ ID NO: 195. In some embodiments, the multispecific molecule comprises the amino acid sequence of SEQ ID NO: 194 and the amino acid sequence of SEQ ID NO: 193. In some embodiments, the multispecific molecule comprises the amino acid sequence of SEQ ID NO: 194 and the amino acid sequence of SEQ ID NO: 195.

In some embodiments, the multispecific molecule comprises a heavy chain constant region 1 (CH1) and a light chain constant region (CL). In some embodiments, (i) the first TGF-beta receptor polypeptide is linked, e.g., via a linker, to the CH1, e.g., the N-terminus of the CH1, and (ii) the second TGF-beta receptor polypeptide is linked, e.g., via a linker, to the CL, e.g., the N-terminus of the CL. In some embodiments, the first TGF-beta receptor polypeptide and the second TGF-beta receptor polypeptide form a homodimer or heterodimer, e.g., a homodimer. In some embodiments, the first or second TGF-beta receptor polypeptide comprises an extracellular domain of TGFBR1, TGFBR2, or TGFBR3, e.g., an extracellular domain of TGFBR2. In some embodiments, the multispecific molecule has the configuration of FIG. 35C or 35D.

In some embodiments, the multispecific molecule comprises the amino acid sequence of SEQ ID NO: 196 and the amino acid sequence of SEQ ID NO: 198. In some embodiments, the multispecific molecule comprises the amino acid sequence of SEQ ID NO: 196 and the amino acid sequence of SEQ ID NO: 199. In some embodiments, the multispecific molecule comprises the amino acid sequence of SEQ ID NO: 197 and the amino acid sequence of SEQ ID NO: 198. In some embodiments, the multispecific molecule comprises the amino acid sequence of SEQ ID NO: 197 and the amino acid sequence of SEQ ID NO: 199.

In some embodiments, the TGF inhibitor is linked, e.g., via a linker, to the anti-CSF1R binding moiety (e.g., an anti-CSF1R antibody molecule) or the anti-CCR2 binding moiety (e.g., an anti-CCR2 antibody molecule). In some embodiments, the anti-CSF1R binding moiety and/or the anti-CCR2 binding moiety comprises an Fc region, wherein the TGF inhibitor is linked, e.g., via a linker, to the Fc region, e.g., the C-terminus of the Fc region. In some embodiments, the multispecific molecule comprises a first TGF-beta inhibitor and a second TGF-beta inhibitor, wherein the first TGF-beta inhibitor is linked, e.g., via a linker, to the anti-CSF1R binding moiety (e.g., an anti-CSF1R antibody molecule) and the second TGF-beta inhibitor is linked, e.g., via a linker, to the anti-CCR2 binding moiety (e.g., an anti-CCR2 antibody molecule). In some embodiments, the anti-CSF1R binding moiety comprises a first Fc region, the anti-CCR2 binding moiety comprises a second Fc region, and the multispecific molecule comprises a first TGF-beta inhibitor and a second TGF-beta inhibitor, wherein the first TGF-beta inhibitor is linked, e.g., via a linker, to the first Fc region, e.g., the C-terminus of the first Fc region, and the second TGF-beta inhibitor is linked, e.g., via a linker, to the second Fc region, e.g., the C-terminus of the second Fc region.

In some embodiments, (i) the anti-CSF1R binding moiety comprises a first light chain and a first heavy chain, wherein the first heavy chain comprises a first Fc region, wherein the C-terminus of the first Fc region is linked to an extracellular domain of TGFBR2 or a sequence substantially identical thereto (e.g., a sequence that is at least 80%, 85%, 90%, or 95% identical thereto); and (ii) the anti-CCR2 binding moiety comprises a second light chain and a second heavy chain, wherein the second heavy chain comprises a second Fc region, wherein the C-terminus of the second Fc region is linked to an extracellular domain of TGFBR2 or a sequence substantially identical thereto (e.g., a sequence that is at least 80%, 85%, 90%, or 95% identical thereto). In some embodiments, the multispecific molecule has the configuration shown in FIG. 12B.

In some embodiments, the multispecific molecule comprises a molecule disclosed in Table 34 or Table 28. In some embodiments, the multispecific molecule has or comprises a configuration shown in any of FIG. 39F or 12A-12J.

In some embodiments, the multispecific molecule disclosed herein) induces antibody dependent cellular cytotoxicity (ADCC).

In some embodiments, the multispecific molecule disclosed herein) does not induce antibody dependent cellular cytotoxicity (ADCC).

In another aspect, provided herein are isolated nucleic acid molecules encoding the multispecific molecule (e.g., antibody) disclosed herein.

In another aspect, provided herein are isolated nucleic acid molecules, which comprises the nucleotide sequence encoding any of the multispecific molecules described herein, or a nucleotide sequence substantially homologous thereto (e.g., at least 95% to 99.9% identical thereto).

In another aspect, provided herein are vectors, e.g., expression vectors, comprising one or more of the nucleic acid molecules described herein.

In another aspect, provided herein are cells, e.g., host cells, comprising the nucleic acid molecule described herein or the vector described herein.

In another aspect, provided herein are methods of making, e.g., producing, the multispecific molecules described herein, comprising culturing the cell, e.g., the host cell, described herein, under suitable conditions, e.g., conditions suitable for gene expression and/or heterodimerization.

In another aspect, provided herein are pharmaceutical compositions comprising the multispecific molecule described herein and a pharmaceutically acceptable carrier, excipient, or stabilizer.

In another aspect, provided herein are methods of treating a hyperproliferative disorder, a cancer, a fibrotic disorder or condition, an inflammatory disorder or condition, or an autoimmune disorder. In one embodiment, the disorder is a hyperproliferative disorder, e.g., a hyperproliferative connective tissue disorder (e.g., a hyperproliferative fibrotic disease). In one embodiment, the fibrotic (e.g., hyperproliferative fibrotic) disease is multisystemic or organ-specific. Exemplary fibrotic diseases include, but are not limited to, multisystemic (e.g., systemic sclerosis, multifocal fibrosclerosis, sclerodermatous graft-versus-host disease in bone marrow transplant recipients, nephrogenic systemic fibrosis, scleroderma), and organ-specific disorders (e.g., fibrosis of the lung, liver, heart, kidney, pancreas, skin and other organs). In other embodiments, the fibrotic disease is chosen from liver fibrosis (e.g., liver cirrhosis, NASH, and other conditions described herein), pulmonary fibrosis (e.g., IPF), renal fibrosis, or fibrosis of the bone marrow (e.g., myelofibrosis).

In another aspect, provided herein are methods of treating a cancer in a subject, comprising administering to the subject in need thereof the multispecific molecule described herein, wherein the multispecific molecule is administered in an amount effective to treat the cancer.

In some embodiments, the cancer is a solid tumor cancer or a metastatic lesion. In some embodiments, the solid tumor cancer is one or more of pancreatic cancer (e.g., pancreatic adenocarcinoma), breast cancer, colorectal cancer, lung cancer (e.g., small or non-small cell lung cancer), skin cancer (e.g., melanoma), ovarian cancer, liver cancer, brain cancer (e.g., glioma), bladder cancer, cervix cancer, head and neck cancer, kidney cancer, mesothelium cancer, thyroid cancer, or uterus cancer.

In some embodiments, the methods further comprise administering a second therapeutic treatment. In some embodiments, the second therapeutic treatment comprises a therapeutic agent (e.g., a chemotherapeutic agent, a biologic agent, hormonal therapy), radiation, or surgery. In some embodiments, the therapeutic agent is selected from: a chemotherapeutic agent, or a biologic agent. In some embodiments, the therapeutic agent is a checkpoint inhibitor. In some embodiments, the check point inhibitor is selected from the group consisting of an anti-CTLA4 antibody, an anti-PD1 antibody (e.g., Nivolumab, Pembrolizumab or Pidilizumab), an anti-PD-L1 antibody, an anti-PD-L2 antibody, an anti-TIM3 antibody, an anti-LAG3 antibody, an anti-CD160 antibody, an anti-2B4 antibody, an anti-CD80 antibody, an anti-CD86 antibody, an anti-B7-H3 (CD276) antibody, an anti-B7-H4 (VTCN1) antibody, an anti-HVEM (TNFRSF14 or CD270) antibody, an anti-BTLA antibody, an anti-KIR antibody, an anti-MHC class I antibody, an anti-MHC class II antibody, an anti-GAL9 antibody, an anti-VISTA antibody, an anti-BTLA antibody, an anti-TIGIT antibody, an anti-LAIR1 antibody, and an anti-A2aR antibody. In some embodiments, the check point inhibitor is an anti-PD1 antibody.

In one aspect, provided herein are methods of treating a cancer in a subject, comprising administering to the subject in need thereof an effective amount of a multispecific molecule described herein (e.g., the anti-CSF1R/anti-CCR2 multispecific molecule described herein) in combination with an anti-PD1 antibody.

In one aspect, the invention pertains to the multispecific molecule of the invention, the antibody molecule of the invention, the nucleic acid molecule of the invention, the vector of the invention, the cell of the invention, or the pharmaceutical composition of the invention for use as a medicament.

In one aspect, the invention pertains to the multispecific molecule of the invention, the antibody molecule of the invention, the nucleic acid molecule of the invention, the vector of the invention, the cell of the invention, or the pharmaceutical composition of the invention for use as a medicament in the treatment of cancer.

In one aspect, provided herein are methods of treating a fibrotic disease or disorder in a subject in need thereof, comprising administering to the subject an effective amount of a multispecific molecule disclosed herein, thereby treating the fibrotic disease or disorder. In some embodiments, the fibrotic disease or disorder is a fibrotic disease or disorder of the lung, the liver, the heart or vasculature, the kidney, the pancreas, the skin, the gastrointestinal tract, the bone marrow or a hematopoietic tissue, the nervous system, the eye, or a combination thereof. In some embodiments, the fibrotic disease or disorder is lung fibrosis (e.g., Idiopathic pulmonary fibrosis (IPF)) or liver fibrosis (e.g., Nonalcoholic steatohepatitis (NASH)).

In some embodiments, treatment of a fibrotic condition includes reducing or inhibiting one or more of: formation or deposition of tissue fibrosis; reducing the size, cellularity (e.g., fibroblast or immune cell numbers), composition; or cellular content, of a fibrotic lesion; reducing the collagen or hydroxyproline content, of a fibrotic lesion; reducing expression or activity of a fibrogenic protein; reducing fibrosis associated with an inflammatory response; decreasing weight loss associated with fibrosis; or increasing survival.

In some embodiments, the fibrotic disease or disorder is primary fibrosis. In one embodiment, the fibrotic disease or disorder is idiopathic. In other embodiments, the fibrotic disease or disorder is associated with (e.g., is secondary to) a disease (e.g., an infectious disease, an inflammatory disease, an autoimmune disease, a malignant or cancerous disease, and/or a connective disease); a toxin; an insult (e.g., an environmental hazard (e.g., asbestos, coal dust, polycyclic aromatic hydrocarbons), cigarette smoking, a wound); a medical treatment (e.g., surgical incision, chemotherapy or radiation), or a combination thereof.

In one aspect, provided herein are methods of treating a liver disease or disorder in a subject in need thereof, comprising administering to the subject an effective amount of a multispecific molecule disclosed herein, thereby treating the liver disease or disorder. In some embodiments, the liver disease or disorder is a fibrotic disorder or connective tissue disorder affecting the function or physiology of the liver. In one embodiment, the fibrotic disorder or connective tissue disorder can be systemic (affecting the whole body), multi-organ, or organ-specific (e.g., liver-specific). Examples of fibrotic liver disorders include, but are not limited to, liver fibrosis (hepatic fibrosis), liver cirrhosis, and any disorder associated with accumulation of extracellular matrix proteins, e.g., collagen, in the liver, liver scarring, and/or abnormal hepatic vasculature. In one embodiment, the liver disease or disorder is liver cirrhosis. Liver cirrhosis is considered to be an end stage of liver fibrosis, involving regenerative nodules (as a result of repair processes), and is typically accompanied with the distortion of the hepatic vasculature. In other embodiments, the liver disease or disorder is a liver cancer. Examples of liver cancers include, but are not limited to, hepatocellular carcinoma (HCC), primary liver cell carcinoma, hepatoma, fibrolamellar carcinoma, focal nodular hyperplasia, cholangiosarcoma, intrahepatic bile duct cancer, angiosarcoma or hemangiosarcoma, hepatic adenoma, hepatic hemangiomas, hepatic hamartoma, hepatoblastoma, infantile hemangioendothelialoma, mixed tumors of the liver, tumors of mesenchymal tissue, and sarcoma of the liver. Liver cancers can also be associated with metastasis of non-liver cancers, such as breast cancer, colorectal cancer, esophageal cancer, kidney or renal cancer, lung cancer, ovarian cancer, pancreatic cancer, rectal cancer, skin cancer (e.g., melanoma), gastric or stomach cancer (including gastrointestinal cancer), and uterine cancer. In one embodiment, the liver disease or disorder is HCC. In certain embodiments, the liver disease or disorder is caused by one or more insults including, but not limited to, liver inflammation or damage; viral (e.g., chronic viral) infection (e.g., hepatitis B, hepatitis C virus, hepatitis A virus, hepatitis D virus (hepatitis delta virus), hepatitis E virus, Epstein-Barr adenovirus, or cytomegalovirus; or parasitic infection, such as schistosomiasis); alcoholism; fatty liver disease; metabolic disorders (e.g., hemachromatosis, diabetes, obesity, hypertension, dyslipidemia, galactosemia, or glycogen storage disease); autoimmune disorders (e.g., autoimmune hepatitis (AIH), autoimmune liver disease, lupoid hepatitis, systemic lupus erythematosus, primary biliary cirrhosis (PBC), scleroderma, or systemic scerlosis); inflammatory liver disorders (e.g., steatohepatitis, primary sclerosing cholangitis (PSC), ulcerative colitis, Crohn's disease, inflammatory bowel disease); inherited or congenital liver disease (e.g., Wilson's disease, Gilbert's disease, Byler syndrome, Greenland-Eskimo familial cholestasis, Zellweger's syndrome, Alagilles syndrome (ALGS), progressive familial intrahepatic cholestasis (PFIC), alpha 1-antitrypsin deficiency, cystic fibrosis, Indian childhood cirrhosis, or hereditary hemochromatosis); and liver injury (e.g., drug toxicity, alcoholism, ischemia, malnutrition, or physical trauma). In one embodiment, the liver disease or disorder is fatty liver (or FLD), alcoholic liver disease, non-alcoholic fatty liver disease (NAFLD), non-alcoholic steatohepatitis (NASH), alcoholic steatohepatitis, simple steatosis, Reye's syndrome, and any disorder associated with abnormal retention of lipids in liver cells.

In one aspect, provided herein are methods of treating an inflammatory disorder or condition in a subject in need thereof, comprising administering to the subject an effective amount of a multispecific molecule disclosed herein, thereby treating the inflammatory disorder or condition. In one embodiment, the inflammatory disorder or condition is an inflammatory disorder or condition in the kidney. In one embodiment, the inflammatory disorder or condition is Lupus nephritis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. In vitro binding of increasing concentrations of UniTI-01 to transiently transfected ExpiCHO cells expressing mCCR2 (circles), mCSF1R (squares), or both mCCR2 and mCSF1R (triangles), as determined by flow analysis. In FIG. 1, percent fluorescence (%) is plotted against antibody concentrations tested.

FIG. 2. Effect of increasing concentrations of isotype control (mIgG2a), anti-mouse CCR2 monoclonal antibody (aCCR2) or UniTI-01 on MCP-1-dependent cell migration of bone marrow-derived monocytes in a trans-well cell culture system.

FIGS. 3A and 3B. Effect of increasing concentrations of UniTI-01, anti-mouse CSF1R monoclonal antibody (aCSF1R), or isotype control (mIgG2a) on mCSF-1-dependent differentiation and proliferation of bone marrow-derived monocytes into macrophages in vitro. FIG. 3A is a pair of flow cytometry plots showing the staining of bone marrow cells with an anti-CCR2 antibody and an anti-CSF1R antibody at day 0 or day 4 of cell culture in the presence of mCSF1. In FIG. 3B, % proliferation is plotted for each condition tested. All antibodies were tested at 15 μg/ml.

FIG. 4. Effect of UniTI-01, anti-mouse CSF1R monoclonal antibody (aCSF1R), or isotype control (mIgG2a) on mCSF-1-dependent differentiation and proliferation of bone marrow-derived monocytes. All antibodies were tested at 15 μg/ml.

FIG. 5. In vitro binding of increasing concentrations of UniTI-01 to primary intratumoral M-MDSCs and M2-like macrophages, as determined by flow analysis. FIG. 5 is a panel of histograms showing staining of CD206+ macrophages, M-MDSCs, neutrophils, or CD3+ T cells using UniTI-01.

FIG. 6. Effect of in vivo administration of UniTI-01 on intratumoral myeloid cell populations in EMT6 and MC38 syngeneic tumor models.

FIGS. 7A-7D. Effect of in vivo administration of UniTI-01 on Kupffer cells in EMT6 syngeneic tumor model. FIGS. 7A and 7B are immunohistochemistry graphs of tumor and liver tissues, respectively, stained with the antibody F4/80. FIGS. 7C and 7D are graphs showing % F4/80 positive area for tumor and liver tissues, respectively.

FIGS. 7E-7H. Effect of in vivo administration of UniTI-01 on healthy tissue macrophages in small intestine and kidney. FIGS. 7E and 7G are immunohistochemistry graphs of small intestine and kidney, respectively, stained with the antibody F4/80. FIGS. 7F and 7H are graphs showing % F4/80 positive area for small intestine and kidney, respectively.

FIGS. 8A and 8B. Effect of UniTI-01 on CSF-1 dependent cell survival in CCR2-negative NFS-60 cells in vitro. In FIG. 8A, cell viability is plotted against the antibody concentrations tested for the anti-CCR2/anti-CSF1R bispecific antibody UniTI-01, a bivalent monospecific anti-CSF1R antibody (aCSF1R), or a monovalent monospecific anti-CSF1R antibody (mono-aCSF1R). FIG. 8B is a pair of flow cytometry plots showing the staining of NFS-60 cells with an anti-CCR2 antibody and an anti-CSF1R antibody, or the staining of NFS-60 cells with an isotype control antibody.

FIG. 9. Effect of in vivo administration of UniTI-01 on CD8+ T cell infiltration in the tumor of EMT6 model. FIG. 9 is a graph showing % CD8+ T cells in CD3+ T cells in tumors under indicated treatments.

FIGS. 10A and 10B. Effect of in vivo administration of UniTI-01 on Treg frequency in the tumor of MC38 model. FIG. 10A is a graph showing % FOXP3+ cells in CD4+ T cells in tumors under indicated treatments. In FIG. 10B, the CD8+ T cell/Treg ratio in tumors is plotted for each treatment.

FIGS. 11A, 11B, and 11C. FIGS. 11A and 11B: UniTI-01 shows antitumor efficacy, tumor regressions and enhanced survival when treated in combination with anti-PDL1 antibody in EMT6 tumor model. FIG. 11A is a panel of graphs showing tumor volumes for each treatment. FIG. 11B is a graph showing percent survival under indicated treatments. FIG. 11C: UniTI-01 shows superior anti-PDL1 combination benefit over anti-CSF1R antibody in MC38 colon model. FIG. 11C is a panel of graphs showing tumor volume for each treatment.

FIGS. 12A-12J. FIG. 12A shows an exemplary configuration of anti-CSF1R/anti-CCR2 bispecific antibody. In one embodiment, the anti-CSF1R/anti-CCR2 bispecific antibody comprises an anti-CSF1R binding moiety and an anti-CCR2 binding moiety. In one embodiment, the anti-CSF1R binding moiety comprises a heavy chain and a kappa light chain, and the anti-CCR2 binding moiety comprises a heavy chain and a lambda light chain. In one embodiment, the anti-CSF1R binding moiety comprises a heavy chain and a lambda light chain, and the anti-CCR2 binding moiety comprises a heavy chain and a kappa light chain. FIG. 12B shows an exemplary configuration of anti-CSF1R/anti-CCR2 bispecific antibody fused to TGFβ Trap. In one embodiment, the TGFβ Trap comprises a homodimer of two extracellular domains of TGFβ receptor 2. FIGS. 12C and 12D show exemplary configurations of anti-CSF1R/anti-CCR2 bispecific antibody fused to an anti-PDL1 binding moiety. In one embodiment, the anti-PDL1 binding moiety is an scFv. FIGS. 12E-12G show exemplary configurations of anti-CSF1R/anti-CCR2 bispecific antibody fused to one or more IL-2 molecules. In one embodiment, the one or more IL-2 molecules are fused to the C-terminus of the light chain of the anti-CSF1R binding moiety or the anti-CCR2 binding moiety. FIG. 12H shows an exemplary configuration of anti-CSF1R/anti-CCR2 bispecific antibody. In one embodiment, the anti-CSF1R/anti-CCR2 bispecific antibody comprises an anti-CSF1R binding moiety and an anti-CCR2 binding moiety. In one embodiment, the anti-CSF1R binding moiety comprises an anti-CSF1R scFv fused to an Fc. In one embodiment, the anti-CCR2 binding moiety comprises an anti-CCR2 Fab fused to an Fc. FIG. 12I shows an exemplary configuration of anti-CSF1R/anti-CCR2 bispecific antibody, additional comprising TGFβ Trap. In one embodiment, the anti-CSF1R/anti-CCR2 bispecific antibody comprises an anti-CSF1R binding moiety and an anti-CCR2 binding moiety. In one embodiment, the anti-CCR2 binding moiety comprises an anti-CCR2 Fab fused to an Fc. In one embodiment, the anti-CSF1R binding moiety comprises an anti-CSF1R scFv fused to the C-terminus of the Fc of the anti-CCR2 binding moiety. In one embodiment, the TGFβ Trap comprises a first TGFBR2 ECD fused to CH1 and Fc, and a second TGFBR2 ECD fused to CL. FIG. 12J shows an exemplary configuration of anti-CSF1R/anti-CCR2 bispecific antibody fused to TGFβ Trap. In one embodiment, the anti-CSF1R/anti-CCR2 bispecific antibody comprises an anti-CSF1R binding moiety and an anti-CCR2 binding moiety. In one embodiment, the anti-CSF1R binding moiety comprises an anti-CSF1R scFv fused to an Fc. In one embodiment, the anti-CCR2 binding moiety comprises an anti-CCR2 Fab fused to an Fc. In one embodiment, the TGFβ Trap comprises a first TGFBR2 ECD fused to the C-terminus of the Fc of the anti-CCR2 binding moiety, and a second TGFBR2 ECD fused to the C-terminus of the Fc of the anti-CSF1R binding moiety.

FIG. 13. FIG. 13 is a schematic showing that anti-CSF1R/anti-CCR2 antibody molecule may reduce immunosuppressive myeloid cells and increase infiltration of cytotoxic T cells in the tumor.

FIG. 14. FIG. 14 is a panel of graphs showing that UniTI-01 spares CSF1R-expressing osteoclasts when compared with anti-CSF1R treatment.

FIGS. 15A and 15B. FIG. 15A is a panel of graphs showing #M-MDSC per mg tumor in mice bearing EMT6, MC38, or LLC1 tumors. FIG. 15B is a panel of graphs showing #TAM per mg tumor in mice bearing EMT6, MC38, or LLC1 tumors.

FIG. 16. FIG. 16 is a panel of graphs showing immunohistochemistry staining of tissues using an anti-rat IgG antibody and an anti-F4/80 antibody.

FIG. 17. FIG. 17 is a bar graph showing biodistribution of UniTI-01 in spleen, brain, lung, heart, colon, kidney, bone, muscle, or tumor at the indicated time points after injection.

FIG. 18. FIG. 18 is a panel of flow cytometry plots showing staining of tumor M-MDSCs or tumor G-MDSCs using an anti-CSF1R antibody and an anti-CCR2 antibody.

FIGS. 19A and 19B. FIG. 19A is a panel of graphs showing immunochemistry staining of tumor tissues using an anti-CSF1R bivalent monospecific antibody, an anti-CCR2 bivalent monospecific antibody, or UniTI-01. FIG. 19B is a graph showing % CD3 Positive Area under each condition tested.

FIG. 20. FIG. 20 is a panel of graphs showing % CD8 positive area, % FoxP3 positive area, or CD8/Treg ratio under each condition tested.

FIG. 21. FIG. 21 is a graph comparing the property of anti-CCR2 bivalent monospecific antibody, anti-CSF1R bivalent monospecific antibody, and UniTI-01, based on in vivo studies using EMT6, MC38 and LLC1 syngeneic tumor models.

FIGS. 22A and 22B. UniTI-01 drives tumor regression and depletes suppressive myeloid cells. FIG. 22A is a pair of graphs showing % M-MDSC of CD45+ cells (upper panel) or % TAM of CD45+ cells (lower panel) under each condition tested. FIG. 22B is a panel of graphs showing tumor volume of mice bearing EMT6 breast tumor under each condition tested. Similar results were observed with mice bearing CT26 and MC38 colon tumors.

FIG. 23. Durable responses and immune memory with UniTI-01/aPD-L1 combination. FIG. 23, left panel, is a pair of graphs showing tumor volume of mice bearing EMT or CT26 tumor. FIG. 23, right panel, is a pair of graphs showing tumor volume of mice re-challenged with both EMT and CT26 tumor cells.

FIGS. 24A, 24B, and 24C. UniTI-01 inhibits tumor growth in additional mouse syngeneic cancer models. FIGS. 24A, 24B, and 24C are a panel of graphs showing tumor volume of mice bearing MC38, CT26, or EMT6 tumors under each condition tested.

FIG. 25. FIG. 25 is a schematic showing that UniTI-102 reduces immunosuppressive myeloid cells and neutralizes TGFβ.

FIGS. 26A and 26B. FIG. 26A is a schematic showing the configuration of UniTI-102. FIG. 26B is a graph showing the level of TGFβ/Smad activation under each condition tested.

FIGS. 27A, 27B, and 27C. UniTI-102 shows strong monotherapy response in EMT6 tumor model and is not enhanced with the addition of anti-PD-L1. FIGS. 27A-27C are a panel of graphs showing tumor volume of mice bearing EMT6 tumor under each condition tested.

FIG. 28 is a pair of SDS gels of anti-CSF1R antibodies.

FIGS. 29A and 29B are a pair of graphs showing binding of anti-human CSF1R antibodies to human and cyno CSF1R-Fc ECD, as measured by ELISA.

FIG. 30 is a graph showing the level of MCP-1 secretion by monocytes in the presence of various monovalent anti-CSF1R antibodies. Data shown is a representative of 1 of 2 human donors.

FIG. 31 is a graph showing binding of monovalent anti-CSF1R antibodies to cells expressing CSF1R, as measured by flow cytometry.

FIG. 32 is a pair of SDS gels of exemplary CCR2×CSF1R bispecific antibodies.

FIG. 33 is a graph showing that CCR2×CSF1R bispecific antibodies block CCL2-dependent reporter activity in Tango CCR2-bla U2OS cells.

FIG. 34 is a graph showing that CCR2×CSF1R bispecific antibodies block hCSF1-mediated CCL2 (MCP-1) secretion in purified human monocytes. Data shown is a representative of 1 of 2 human donors.

FIGS. 35A-35D are schematics showing exemplary multispecific molecules comprising a TGFβ inhibitor. In some embodiments, the TGFβ inhibitor comprises a TGF-beta receptor ECD homodimer. In some embodiments, the TGFβ inhibitor comprises a TGFBR2 ECD heterodimer. In FIGS. 35A and 35B, the two TGFBR ECD domains are linked to the C-terminus of two Fc regions. In some embodiments, the CH1-Fc-TGFBR ECD region shown in FIG. 35A or 35B comprises the amino acid sequence of SEQ ID NO: 192 or 193. In some embodiments, the Fc-TGFBR ECD region shown in FIG. 35A or 35B comprises the amino acid sequence of SEQ ID NO: 194 or 195. In FIGS. 35C and 35D, the two TGFBR ECD domains are linked to CH1 and CL, respectively. In some embodiments, the TGFBR ECD-CH1-Fc region shown in FIG. 35C or 35D comprises the amino acid sequence of SEQ ID NO: 196 or 197. In some embodiments, the TGFBR ECD-CL region shown in FIG. 35C or 35D comprises the amino acid sequence of SEQ ID NO: 198 or 199. In some embodiments, the multispecific molecule comprises binding a moiety A and a binding moiety B. In some embodiments, the binding moiety A or binding moiety B is an anti-CSF1R binding moiety (e.g., an anti-CSF1R antibody molecule). In some embodiments, the binding moiety A or binding moiety B is an anti-CCR2 binding moiety (e.g., an anti-CCR2 antibody molecule). In some embodiments, the binding moiety A is an anti-CSF1R binding moiety (e.g., an anti-CSF1R antibody molecule) and the binding moiety B is an anti-CCR2 binding moiety (e.g., an anti-CCR2 antibody molecule). In some embodiments, the binding moiety A is an anti-CCR2 binding moiety (e.g., an anti-CCR2 antibody molecule) and the binding moiety B is an anti-CSF1R binding moiety (e.g., an anti-CSF1R antibody molecule).

FIG. 36 is a graph in which TGFβ/Smad activation is plotted against TGFβ-trap concentrations. Constructs tested in this study included: Single TGFβ Fab-trap, Anti-PDL1×TGFβ-trap, UniTI-01 (Anti-CCR2×anti-CSF1R), and UniTI-102 (Anti-CCR2×anti-CSF1R×TGFβ-trap).

FIG. 37. CSF1R and CCR2 co-expression on monocytic myeloid-derived suppressor cells (Mo-MDSCs) and tumor-associated macrophages (TAMs) from cancer patients. FIG. 37 includes a panel of flow cytometry plots showing staining of mo-MDSCs or TAMs from ovarian tumors (upper panel) or colorectal tumors (lower panel) using an anti-CSF1R antibody and an anti-CCR2 antibody. FIG. 37 also includes bar graphs showing % CCR2+ CSF1R+ cells among M-MDSCs, TAMs, granulocytic myeloid-derived suppressor cells (G-MDSCs), CD4+ T cells, CD8+ T cells, monocytes, or non-immune cells from ovarian tumors (upper panel) or colorectal tumors (lower panel). CSF1R and CCR2 co-expression was also detected on CD163+ TAMs.

FIGS. 38A-38D. Therapeutic concept demonstrated with mouse surrogate mUniTI-102 in a syngeneic tumor model resistant to PD1 or TGFβ blockade therapy. FIG. 38A is a graph showing that mUniTI-102 depletes mo-MDSCs in EMT6 breast tumors. FIG. 38B is a graph showing that mUniTI-102 preferentially depletes TAMs over tissue-resident macrophages. FIG. 38C is a graph showing that mUniTI-102 demonstrates monotherapy anti-tumor activity in the EMT6 breast tumor model. FIG. 38D is a graph showing that mUniTI-102 enhances survival in EMT6 tumor-bearing mice.

FIGS. 39A-39F. Human UniTI-102 preferentially binds to primary human classical monocytes in whole blood. FIG. 39A is a cartoon showing CCR2 and CSF1R co-expression on a classical monocyte. FIG. 39B is a graph showing normalized frequency of UniTI-102+ cells among classical monocytes at the indicated concentrations of BIM0648 (“648”), BIM0652 (“052”), or an isotype control antibody (“iso”). FIG. 39C is a cartoon showing CSF1R expression on a non-classical monocyte. FIG. 39D is a graph showing normalized frequency of UniTI-102+ cells among non-classical monocytes (CD14-CD16+) at the indicated concentrations of BIM0648 (“648”), BIM0652 (“052”), or an isotype control antibody (“iso”). FIG. 39E is a table showing receptor density of CCR2 and CSF1R on different types of monocytes. FIG. 39F shows the configuration of UniTI-102 molecules BIM0648 and BIM0652.

FIGS. 40A-40F. TGFβ neutralization by human UniTI-102 molecules confirmed by various functional assays in vitro. In FIG. 40A, TGFβ/Smad activation is plotted against concentrations of BIM0648, BIM0652, and a human IgG1. FIG. 40B is a graph showing % FOXP3+CD25+ of CD4+ T cells for the following treatment groups: with TGFβ but without any constructs (+TGFb), without TGFβ (No TGFb), UniTI-102 molecule BIM0648, anti-PD-L1-TGFBR2, and human IgG1. FIG. 40C is a graph showing specific killing for the following treatment groups: with TGFβ but without any constructs (+TGFb), without TGFβ (No TGFb), UniTI-102 molecule BIM0648, anti-PD-L1-TGFBR2, and human IgG1. FIG. 40D is a graph showing the amount of TGFb1, TGFb2, TGFb3, IL-10, IL-4, and IL-6 secreted by SW840 cells. FIGS. 40E and 40F are graphs showing the expression of CD206 (a M2 marker) and HLA (a M1 marker), respectively, in each treatment group. Data is representative of 2 donors. Comparable results were observed for BIM0652.

FIGS. 41A and 41B. Pharmacokinetic data for BIM0648 in healthy cynomolgus monkeys. FIG. 41A is a graph showing the concentration of BIM0648 at the indicated time points after administration into healthy cynomolgus monkeys at 0.1 mg/kg, 1 mg/kg, 10 mg/kg, or 30 mg/kg. FIG. 41B is a table summarizing pharmacokinetic data for BIM0648.

FIG. 42. Evidence of target occupancy for BIM0648 in cynomolgus monkeys. FIG. 42 is a panel of graphs showing serum levels of TGFβ1 (left), M-CSF (middle), and MCP-1 (right) in cynomolgus monkeys after administration of BIM0648 at 0.1 mg/kg, 1 mg/kg, 10 mg/kg, or 30 mg/kg.

FIGS. 43A-43D. Trend of biological response noted for BIM0648, consistent with CSF1R blockade in monkey and human. FIGS. 43A-43D are a panel of graphs showing non-classical monocytes (% total monocytes) in cynomolgus monkeys at the indicated time points after administration of BIM0648 at 0.1 mg/kg (FIG. 43A), 1 mg/kg (FIG. 43B), 10 mg/kg (FIG. 43C), or 30 mg/kg (FIG. 43D).

FIG. 44. Murine CCR2×CSF1R bispecific molecule (mUniTI-01) is more effective in depleting circulating Mo-MDSCs in EMT6 tumor-bearing mice compared to anti-CSF1R mAb. FIG. 44 is a bar graph showing % M-MDSC in EMT6 tumor-bearing mice after administration of mUniTI-01 or an anti-CSF1R antibody (“CSF1R”) in combination with an anti-PDL1 antibody.

FIGS. 45A and 45B. mUniTI-102 efficiently binds to blood Ly6C^{hi} monocytes in EMT6 syngeneic tumor model. FIG. 45A includes flow cytometry histograms showing the binding of mUniTI-102 to Ly6C^{hi} classical monocytes. FIG. 45B is a graph showing % TGFβRII+Ly6C^{hi} monocytes as detected using an anti-human TGFβRII antibody.

FIGS. 46A and 46B. mUniTI-102 depletes blood Ly6C^{hi} classical monocytes in EMT6 syngeneic tumor model. FIGS. 46A and 46B are graphs showing % Ly6C^{hi} monocytes of single cells and % Ly6C^{lo} monocytes of single cells, respectively, in EMT6 tumor-bearing mice after administration of four doses of mUniTI-102. Administration of mUniTI-102 led to dose-dependent depletion of Ly6C^{hi} classical monocytes in blood while sparing Ly6C^{lo} non-classical monocytes, which are CCR2 negative. Minimal changes were noted in whole blood % CD3+, Treg or CD4/CD8 ratios.

FIGS. 47A and 47B. mUniTI-102 decreases serum TGFβ-1 levels in EMT6 syngeneic tumor model. FIG. 47A is a diagram showing the design of an in vivo study testing mUniTI-102 in a EMT6 syngeneic tumor model. FIG. 47B is a bar graph showing TGF-β1 levels in acid treated serum samples as measured by ELISA. Similar reduction was also observed in a CT26 model.

FIG. 48 is a table showing human and cynomolgus whole blood receptor density for CCR2 and CSF1R. There is higher CSF1R expression on non-classical monocytes in cynomolgus compared to human. Consistent with this expression pattern, unlike human whole blood, significant binding was present to non-classical monocyte populations in cynomolgus monkey whole blood. No significant differences were observed in binding between BIM0648 and BIM0652 to monocyte populations in cynomolgus monkey whole blood.

FIGS. 49A-49F. UniTI-102 (BIM0648) exhibits excellent manufacturability properties. FIG. 49A summarizes product information for BIM0648. FIG. 49B is a graph showing results from a study testing the stability of BIM0648 in PBS at 40° C. on Day 0, Day 10, and Day 30. FIG. 49C is a graph showing high colloidal stability and low aggregation propensity of BIM0648. FIG. 49D is a graph showing results from a study testing the thermal stability of BIM0648. FIG. 49E is a graph showing results from a study testing the propensity of BIM0648 to self-aggregate. FIG. 49F is a graph showing results from a study testing the stability of BIM0648 at pH 3.5.

FIG. 50. UniTI-102 (BIM0648) shows retained binding to Fc receptors. FIG. 50 is a graph showing binding of BIM0648 to Fc receptors as assessed using Biacore.

FIG. 51. UniTI-102 (BIM0648) shows negligible non-specific binding propensity. FIG. 51 is a bar graph showing results from a study testing non-specific binding of BIM0648. Poly-reactivity was assessed by checking binding to baculovirus particles. hIgG1, bevacizumab and pembrolizumab were used as benchmarks.

FIGS. 52A-52D are a panel of graphs showing binding of BIM0648 to M1 macrophages, MDSCs, TAMs, and M2 macrophages. BHM1650, a bivalent anti-CSF1R antibody, was used as a control. In vitro differentiated monocyte-derived M1 macrophages have lowest expression of CSF1R and CCR2. TAMs and M2 macrophages have the highest expression. MDSCs have intermediate CSF1R expression and similar CCR2 expression levels as M1 macrophages. Engagement of BIM0648 correlates with CCR2/CSF1R expression—highest in TAMs and M2 macrophages, intermediate in MDCSs, and no binding in M1 macrophages.

FIGS. 53A and 53B are graphs showing % binding of IgG, BIM0648, and BHM1650 (a bivalent anti-CSF1R antibody) to classical monocytes (FIG. 53A) or non-classical monocytes (FIG. 53B). BIM0648 binds to classical monocytes with greater affinity than to non-classical monocytes in PBMCs.

FIGS. 54A-54C are graphs showing results from an anti-CSF1R function assay: human monocyte survival assay. FIGS. 54A and 54B are graphs showing results from BIM0648, BIM0652, BHM1650 (a bivalent anti-CSF1R antibody), human IgG1, no cytokine group, and CSF1 only group. FIG. 54C is a table showing inhibition of human monocyte survival/proliferation/IC50 (nM) for BIM0648, BIM0652, and BHM1650.

FIGS. 55A-55B are graphs showing results from an anti-CCR2 function assay: human monocytic cell migration assay. FIG. 55A is a graph showing cell count (migration) against antibody concentrations of BIM0648, BIM0652, BHM0139 (a bivalent anti-CCR2 antibody), human IgG1, no rhCCL2 group, and rhCCL2 alone group. FIG. 55B is a table showing IC50 (nM) of BIM0648, BIM0652 and BHM0139 for inhibiting THP-1 migration.

FIGS. 56A and 56B are graphs showing results from a TGFβ trap function assay: a reporter cell assay. FIG. 56A is a graph showing TGFβ-induced Smad activation for BIM0648, BIM0652, BHM1603 (a bivalent anti-PDL1 antibody fused to TGFβ tap at the C-terminus of the Fc region), human IgG1, cells-only group, and cells+human TGFβ1 group. FIG. 56B is a table showing the IC50 (nM) of BIM0648, BIM0652, and BHM1603 for inhibiting TGFβ-induced Smad signal.

FIG. 57 is a graph showing results from a TGFβ trap function assay: human Treg differentiation assay. FIG. 57 includes a panel of flow cytometry plots showing the staining of CD25 and Foxp3. The antibodies tested in the human Treg differentiation assay include: BIM0648, BHM1603 (a bivalent anti-PDL1 antibody fused to TGFβ tap at the C-terminus of the Fc region), BHM1583 (a C-terminal Fc fusion of TGFβRII trap), and human IgG1 isotype antibody.

FIG. 58. UniTI-102 (BIM0648) can effectively inhibit human Treg differentiation in vitro. FIG. 58 is a graph showing % FOXP3+CD25+ cells of CD4+ T cells for the following treatment groups: with TGFβ and without constructs (“+TGFb”), no TGFβ, UniTI-102 (BIM0648), BHM1603 (a bivalent anti-PDL1 antibody fused to TGFβ tap at the C-terminus of the Fc region), BHM1583 (a C-terminal Fc fusion of TGFβRII trap), and human IgG1.

FIG. 59 is a graph illustrating a TGFβ trap function assay: a human NK cell killing assay.

FIGS. 60A-60C. UniTI-102 (BIM0648) reverses suppressive effect of TGFβ on primary human NK cell killing. FIG. 60A is a panel of flow cytometry plots showing results from the human NK cell killing assay. FIGS. 60B and 60C are bar graphs showing K562 killing and NKp30 MFI, respectively, for the indicated treatment groups. The antibodies tested in this study include: IgG isotype control antibody, BIM0648, and BIM1603 (a bivalent anti-PDL1 antibody fused to TGFβ tap at the C-terminus of the Fc region).

FIGS. 61A-61C. UniTI-102 (BIM0648) reverses suppressive effect of TGFβ on primary human NK cell killing. FIG. 61A is a graph showing percent killing. FIGS. 61B and 61C are graphs showing percent CD56+ NKp30+ cells. The following groups were tested in this study: human IgG, BIM0648, BHM1603 (a bivalent anti-PDL1 antibody fused to TGFβ tap at the C-terminus of the Fc region), BHM1583 (a C-terminal Fc fusion of TGFβRII trap), without TGFβ (“NK”), and with TGFβ and without constructs (“TGFb”).

### DETAILED DESCRIPTION OF THE INVENTION

TAMs originate from circulating monocytes and their recruitment into tumors is driven by tumor-derived chemotactic factors. TAMs can promote tumor cell proliferation and metastasis by causing such responses as inhibition of B and T cell activation, inhibition of tumor-associated antigen presentation, inhibition of cytotoxic granule release, increased angiogenesis, and secretion a wide range of growth and proangiogenic factors (see e.g., Liu et al Cellular & Molecular Immunology (2015) 12, 1-4; and Noy, Roy et al Immunity, Volume 41, Issue 1, 49-61; and Quatromoni et al. Am J Transl Res. 2012; 4(4): 376-389). Consequently, many tumors with a high number of TAMs have an increased tumor growth rate, local proliferation and distant metastasis. Thus, therapies that deplete TAMs or inhibit their activity would be useful.

### Definitions

As used herein, the term “transforming growth factor beta-1 (TGF-beta 1)” refers to a protein that in humans is encoded by the gene TGFB1, or its orthologs. Swiss-Prot accession number P01137 provides exemplary human TGF-beta 1 amino acid sequences. An exemplary immature human TGF-beta 1 amino acid sequence is provided in SEQ ID NO: 92. An exemplary mature human TGF-beta 1 amino acid sequence is provided in SEQ ID NO: 117.

As used herein, the term “transforming growth factor beta-2 (TGF-beta 2)” refers to a protein that in humans is encoded by the gene TGFB2, or its orthologs. Swiss-Prot accession number P61812 provides exemplary human TGF-beta 2 amino acid sequences. An exemplary immature human TGF-beta 2 amino acid sequence is provided in SEQ ID NO: 93. An exemplary mature human TGF-beta 2 amino acid sequence is provided in SEQ ID NO: 118.

As used herein, the term “transforming growth factor beta-3 (TGF-beta 3)” refers to a protein that in humans is encoded by the gene TGFB3, or its orthologs. Swiss-Prot accession number P10600 provides exemplary human TGF-beta 3 amino acid sequences. An exemplary immature human TGF-beta 3 amino acid sequence is provided in SEQ ID NO: 94. An exemplary mature human TGF-beta 3 amino acid sequence is provided in SEQ ID NO: 119.

As used herein, a “TGF-beta receptor polypeptide” refers to a TGF-beta receptor (e.g., TGFBR1, TGFBR2, or TGFBR3) or its fragment, or variant thereof.

As used herein, the term “transforming growth factor beta receptor type 1 (TGFBR1)” (also known as ALK-5 or SKR4) refers to a protein that in humans is encoded by the gene TGFBR1, or its orthologs. Swiss-Prot accession number P36897 provides exemplary human TGFBR1 amino acid sequences. Exemplary immature human TGFBR1 amino acid sequences are provided in SEQ ID NOs: 95, 96, and 97. Exemplary mature human TGFBR1 amino acid sequences are provided in SEQ ID NOs: 120, 121, and 122. As used herein, a “TGFBR1 polypeptide” refers to a TGFBR1 or its fragment, or variant thereof.

As used herein, the term “transforming growth factor beta receptor type 2 (TGFBR2)” refers to a protein that in humans is encoded by the gene TGFBR2, or its orthologs. Swiss-Prot accession number P37173 provides exemplary human TGFBR2 amino acid sequences. Exemplary immature human TGFBR2 amino acid sequences are provided in SEQ ID NOs: 98 and 99. Exemplary mature human TGFBR2 amino acid sequences are provided in SEQ ID NOs: 123 and 124. As used herein, a “TGFBR2 polypeptide” refers to a TGFBR2 or its fragment, or variant thereof.

As used herein, the term “transforming growth factor beta receptor type 3 (TGFBR3)” refers to a protein that in humans is encoded by the gene TGFBR3, or its orthologs. Swiss-Prot accession number Q03167 provides exemplary human TGFBR3 amino acid sequences. Exemplary immature human TGFBR3 amino acid sequences are provided in SEQ ID NOs: 106 and 107. Exemplary mature human TGFBR3 amino acid sequences are provided in SEQ ID NOs: 125 and 126. As used herein, a “TGFBR3 polypeptide” refers to a TGFBR3 or its fragment, or variant thereof.

As used herein, the term “variant” of a parent sequence refers to a sequence that has a substantially identical amino acid sequence to the parent sequence, or a fragment thereof. In some embodiments, the variant is a functional variant.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

“Antibody molecule” as used herein refers to a protein, e.g., an immunoglobulin chain or fragment thereof, comprising at least one immunoglobulin variable region sequence. An antibody molecule encompasses antibodies (e.g., full-length antibodies) and antibody fragments. In an embodiment, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)₂ fragments, and single chain variable fragments (scFvs).

As used herein, an “immunoglobulin variable region sequence” refers to an amino acid sequence which can form the structure of an immunoglobulin variable region. For example, the sequence may include all or part of the amino acid sequence of a naturally-occurring variable region. For example, the sequence may or may not include one, two, or more N- or C-terminal amino acids, or may include other alterations that are compatible with formation of the protein structure.

In embodiments, an antibody molecule is monospecific, e.g., it comprises binding specificity for a single epitope. In some embodiments, an antibody molecule is multispecific, e.g., it comprises a plurality of immunoglobulin variable region sequences, where a first immunoglobulin variable region sequence has binding specificity for a first epitope and a second immunoglobulin variable region sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Antigen” (Ag) as used herein refers to a molecule that can provoke an immune response, e.g., involving activation of certain immune cells and/or antibody generation. Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The “antigen-binding site,” or “binding portion” of an antibody molecule refers to the part of an antibody molecule, e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4.

“Cancer” as used herein can encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors.

The compositions and methods of the present invention encompass polypeptides and nucleic acids having the sequences specified, or sequences substantially identical or similar thereto, e.g., sequences at least 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid (e.g., SEQ ID NO: 1) molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof, amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, the term “immunosuppressive myeloid cell” or “IMC” generally refers to a cell of myeloid lineage that promotes immunosuppression (e.g., in a tumor microenvironment) (e.g., by inhibiting T cell activation, inhibiting T cell viability, promoting T regulatory cell induction and recruitment). Immunosuppressive myeloid cells include, e.g., tumor associated macrophages (TAMs) and myeloid derived suppressor cells (MDSCs).

As used herein, the term “tumor associated macrophage” or “TAM” generally refers to a macrophage that exists in the microenvironment of a cancer, for example, a tumor.

As used herein, the term “reducing TAMs” generally refers to decreasing the number of TAMs. Reducing includes decreasing the number of TAMs in a tumor or near a tumor (e.g., as compared to the number of TAMs prior to administration of a multispecific molecule described herein (e.g., prior to 1, 2, 3, 4, 5, 6, 7, 8, 9, 10 or more administrations of a multispecific molecule described herein). Reducing includes decreasing any number of TAMs (e.g., 1%, 5%, 10%, 20%, 30%, 40%, 50%, 60%, 70%, 80%, 90%, 95%, 99%, 100%, all, or substantially) (e.g., as compared to the number of TAMs prior to administration of a multispecific molecule described herein (e.g., prior to 1, 2, 3, 4, 5, 6, 7, 8, 9, 10 or more administrations of a multispecific molecule described herein).

As used herein, the term “myeloid derived suppressor cell” or “MDSC” generally refers to a cell of myeloid origin that is capable of promoting immunosuppression and commonly express CD33, CD11b and CD45. Various subpopulations of MDSCs have been defined, for example monocytic-MDSCs (M-MDSCs) are commonly associated with expression of CD14 and CD124 and low expression of HLA-DR. In some embodiments, the MDSC population is an MO-MDSC population. Polymorphonuclear MDSCs (PMN-MDSCs) are associated with expression of CD15, CD66b, and CD124, and no expression of HLA-DR. Immature MDSCs (I-MDSCs) are associated with expression of CD117 and CD34 and no expression of LIN and HLA-DR. See e.g., Ugel et al. (2015) JCI Vol 125 (9), page 3365.

As used herein, the term “a CSF1R-positive, CCR2-positive cell” refers to a cell expressing both CSF1R and CCR2 on the cell surface. The term “a CSF1R-positive, CCR2-negative cell” refers to a cell expressing CSF1R, but not CCR2 on the cell surface. The term “a CSF1R-negative, CCR2-positive cell” refers to a cell expressing CCR2, but not CSF1R on the cell surface.

As used herein, a binding moiety, e.g., an antibody molecule, binds to a target monovalently, when the binding moiety, e.g., the antibody molecule, binds to a single epitope on the target. In some embodiments, the binding moiety comprises only one antigen binding domain to the target. In some embodiments, one molecule of the binding moiety can only bind to one molecule of the target.

As used herein, a binding moiety, e.g., an antibody molecule, binds to a target bivalently, when the binding moiety, e.g., the antibody molecule, binds to two epitopes on the target. In some embodiments, the two epitopes are identical. In some embodiments, the two epitopes are different. In some embodiments, the binding moiety comprises two antigen binding domains to the target. In some embodiments, one molecule of the binding moiety can bind to two molecules of the target.

Various aspects of the invention are described in further detail below. Additional definitions are set out throughout the specification.

### Antigens

TAM targeting antigens of the present disclosure include, e.g., CSF1R, CCR2, CXCR2, CD68, CD163, CX3CR1, MARCO, CD204, CD52, and folate receptor beta. Exemplary amino acid sequences of TAM targeting antigens are provided herein.

### CSF1R

CSF1R (also known as Macrophage colony-stimulating factor 1 receptor) is a tyrosine-protein kinase that acts as cell-surface receptor for CSF1 and IL34 and plays an essential role in the regulation of survival, proliferation and differentiation of hematopoietic precursor cells, especially mononuclear phagocytes, such as macrophages and monocytes. CSF1R promotes the release of pro-inflammatory chemokines in response to IL34 and CSF1, and thereby plays an important role in innate immunity and in inflammatory processes. Exemplary CSF1R immature amino acid sequences are provided in SEQ ID NOs: 87 and 88.

CSF1R immature amino acid sequence isoform 1
(identifier: P07333-1):
SEQ ID NO: 87
MGPGVLLLLLVATAWHGQGIPVIEPSVPELVVKPGATVTLRCVGNGSVEW
DGPPSPHWTLYSDGSSSILSTNNATFQNTGTYRCTEPGDPLGGSAAIHLY
VKDPARPWNVLAQEVVVFEDQDALLPCLLTDPVLEAGVSLVRVRGRPLMR
HTNYSFSPWHGFTIHRAKFIQSQDYQCSALMGGRKVMSISIRLKVQKVIP
GPPALTLVPAELVRIRGEAAQIVCSASSVDVNFDVFLQHNNTKLAIPQQS
DFHNNRYQKVLTLNLDQVDFQHAGNYSCVASNVQGKHSTSMFFRVVESAY
LNLSSEQNLIQEVTVGEGLNLKVMVEAYPGLQGFNWTYLGPFSDHQPEPK
LANATTKDTYRHTFTLSLPRLKPSEAGRYSFLARNPGGWRALTFELTLRY
PPEVSVIWTFINGSGTLLCAASGYPQPNVTWLQCSGHTDRCDEAQVLQVW
DDPYPEVLSQEPFHKVTVQSLLTVETLEHNQTYECRAHNSVGSGSWAFIP
ISAGAHTHPPDEFLFTPVVVACMSIMALLLLLLLLLLYKYKQKPKYQVRW
KIIESYEGNSYTFIDPTQLPYNEKWEFPRNNLQFGKTLGAGAFGKVVEAT
AFGLGKEDAVLKVAVKMLKSTAHADEKEALMSELKIMSHLGQHENIVNLL
GACTHGGPVLVITEYCCYGDLLNFLRRKAEAMLGPSLSPGQDPEGGVDYK
NIHLEKKYVRRDSGFSSQGVDTYVEMRPVSTSSNDSFSEQDLDKEDGRPL
ELRDLLHFSSQVAQGMAFLASKNCIHRDVAARNVLLTNGHVAKIGDFGLA
RDIMNDSNYIVKGNARLPVKWMAPESIFDCVYTVQSDVWSYGILLWEIFS
LGLNPYPGILVNSKFYKLVKDGYQMAQPAFAPKNIYSIMQACWALEPTHR
PTFQQICSFLQEQAQEDRRERDYTNLPSSSRSGGSGSSSSELEEESSSEH
LTCCEQGDIAQPLLQPNNYQFC
CSF1R immature amino acid sequence isoform 2
(identifier: P07333-2):
SEQ ID NO: 88
MGPGVLLLLLVATAWHGQGIPVIEPSVPELVVKPGATVTLRCVGNGSVEW
DGPPSPHWTLYSDGSSSILSTNNATFQNTGTYRCTEPGDPLGGSAAIHLY
VKDPARPWNVLAQEVVVFEDQDALLPCLLTDPVLEAGVSLVRVRGRPLMR
HTNYSFSPWHGFTIHRAKFIQSQDYQCSALMGGRKVMSISIRLKVQKVIP
GPPALTLVPAELVRIRGEAAQIVCSASSVDVNFDVFLQHNNTKLAIPQQS
DFHNNRYQKVLTLNLDQVDFQHAGNYSCVASNVQGKHSTSMFFRVVGTPS
PSLCPA

### CCR2

CCR2 (also known as C—C chemokine receptor type 2) is a G protein coupled receptor for the CCL2, CCL7 and CCL13 chemokines. CCR2 is known to function in the recruitment of monocytes/macrophages and T cells. CCR2 is expressed is expressed on monocytes and a small subpopulation of T cells and exhibits an almost identical expression pattern in mice and humans (Mack et al. J Immunol 2001; 166:4697-4704). Exemplary CCR2 amino acid sequences are provided in SEQ ID NOs: 89 and 90.

CCR2 amino acid sequence isoform A (Identifier:
P41597-1):
SEQ ID NO: 89
MLSTSRSRFIRNTNESGEEVTTFFDYDYGAPCHKFDVKQIGAQLLPPLYS
LVFIFGFVGNMLVVLILINCKKLKCLTDIYLLNLAISDLLFLITLPLWAH
SAANEWVFGNAMCKLFTGLYHIGYFGGIFFIILLTIDRYLAIVHAVFALK
ARTVTFGVVTSVITWLVAVFASVPGIIFTKCQKEDSVYVCGPYFPRGWNN
FHTIMRNILGLVLPLLIMVICYSGILKTLLRCRNEKKRHRAVRVIFTIMI
VYFLFWTPYNIVILLNTFQEFFGLSNCESTSQLDQATQVTETLGMTHCCI
NPIIYAFVGEKFRSLFHIALGCRIAPLQKPVCGGPGVRPGKNVKVTTQGL
LDGRGKGKSIGRAPEASLQDKEGA
CCR2 amino acid sequence isoform B (Identifier:
P41597-2):
SEQ ID NO: 90
MLSTSRSRFIRNTNESGEEVTTFFDYDYGAPCHKFDVKQIGAQLLPPLYS
LVFIFGFVGNMLVVLILINCKKLKCLTDIYLLNLAISDLLFLITLPLWAH
SAANEWVFGNAMCKLFTGLYHIGYFGGIFFIILLTIDRYLAIVHAVFALK
ARTVTFGVVTSVITWLVAVFASVPGIIFTKCQKEDSVYVCGPYFPRGWNN
FHTIMRNILGLVLPLLIMVICYSGILKTLLRCRNEKKRHRAVRVIFTIMI
VYFLFWTPYNIVILLNTFQEFFGLSNCESTSQLDQATQVTETLGMTHCCI
NPIIYAFVGEKFRRYLSVFFRKHITKRFCKQCPVFYRETVDGVTSTNTPS
TGEQEVSAGL

### CXCR2

CXCR2 (also known as interleukin-8 receptor) is the G protein coupled receptor for IL8 which is a neutrophil chemotactic factor. Binding of IL8 to the receptor causes activation of neutrophils. This response is mediated via a G-protein that activates a phosphatidylinositol-calcium second messenger system. CXCR2 binds to IL-8 with high affinity, and also binds with high affinity to CXCL3, GRO/MGSA and NAP-2. CXCR2 is expressed at high levels on circulating neutrophils and is critical for directing their migration to sites of inflammation (J Clin Invest. 2012; 122(9):3127-3144). An exemplary CXCR2 amino acid sequence is provided in SEQ ID NO: 91.

CXCR2 amino acid sequence (Identifier: P25025-1):
SEQ ID NO: 91
MEDFNMESDSFEDFWKGEDLSNYSYSSTLPPFLLDAAPCEPESLEINKYF
VVIIYALVFLLSLLGNSLVMLVILYSRVGRSVTDVYLLNLALADLLFALT
LPIWAASKVNGWIFGTFLCKVVSLLKEVNFYSGILLLACISVDRYLAIVH
ATRTLTQKRYLVKFICLSIWGLSLLLALPVLLFRRTVYSSNVSPACYEDM
GNNTANWRMLLRILPQSFGFIVPLLIMLFCYGFTLRTLFKAHMGQKHRAM
IRVIFAVVLIFLLCWLPYNLVLLADTLMRTQVIQETCERRNHIDRALDAT
EILGILHSCLNPLIYAFIGQKFRHGLLKILAIHGLISKDSLPKDSRPSFV
GSSSGHTSTTL

### Exemplary Antibodies

Exemplary antibodies binding TAM antigens are provided throughout the specification and below. Exemplary anti-CSF1R antibodies are described herein as well as in WO2009026303A1; WO2011123381A1; WO2016207312A1; WO2016106180A1; US20160220669A1; US20160326254A1; WO2013169264A1; WO2013087699A1; WO2011140249A2; WO2011131407A1; WO2011123381A1; WO2011107553A1; and WO2011070024A1, all of which are herein incorporated by reference in their entirety. Exemplary CCR2 antibodies are described herein as well as in WO2013192596A2; WO2010021697A2; WO2001057226A1; and WO1997031949A1, all of which are herein incorporated by reference in their entirety. Exemplary CXCR2 antibodies are described in WO2014170317A1 and US20160060347 (see e.g., a) SEQ ID NO: 14 (light chain) and SEQ ID NO: 15 (heavy chain); b) SEQ ID NO: 24 (light chain) and SEQ ID NO: 25 (heavy chain); c) SEQ ID NO: 34 (light chain) and SEQ ID NO: 35 (heavy chain); d) SEQ ID NO: 44 (light chain) and SEQ ID NO: 45 (heavy chain); e) SEQ ID NO: 54 (light chain) and SEQ ID NO: 55 (heavy chain); f) SEQ ID NO: 64 (light chain) and SEQ ID NO: 65 (heavy chain); g) SEQ ID NO: 74 (light chain) and SEQ ID NO: 75 (heavy chain); h) SEQ ID NO: 84 (light chain) and SEQ ID NO: 85 (heavy chain)), all of which are herein incorporated by reference in their entirety. Exemplary anti-CD163 antibodies are provided in US20120258107 (see e.g., MAC2158, MAC2-48), herein incorporated by reference in its entirety. Exemplary anti-CD52 antibodies are described in US20050152898, herein incorporated by reference in its entirety. Exemplary anti-folate antibodies are described in U.S. Pat. No. 9,522,196, herein incorporated by reference in its entirety. Exemplary anti-CD52 antibodies are described in US20050152898, herein incorporated by reference in its entirety. Exemplary anti-MARCO antibodies are described in WO2016196612, herein incorporated by reference in its entirety.

### Antibody Molecules

In one embodiment, the multispecific molecule comprises an antibody molecule that binds to a first tumor associated macrophage (TAM) antigen; and an antibody molecule that binds to a second TAM antigen. In some embodiments, the first and/or second TAM antigen is, e.g., a mammalian, e.g., a human. For example, the antibody molecule binds specifically to an epitope, e.g., linear or conformational epitope, on the TAM antigen.

In one embodiment, the multispecific molecule comprises an antibody molecule that binds to a first myeloid derived suppressor cell (MDSC) antigen; and an antibody molecule that binds to a second MDSC antigen. In some embodiments, the first and/or second MDSC antigen is, e.g., a mammalian, e.g., a human. For example, the antibody molecule binds specifically to an epitope, e.g., linear or conformational epitope, on the MDSC antigen.

In an embodiment, an antibody molecule is a monospecific antibody molecule and binds a single epitope. E.g., a monospecific antibody molecule having a plurality of immunoglobulin variable region sequences, each of which binds the same epitope.

In an embodiment an antibody molecule is a multispecific antibody molecule, e.g., it comprises a plurality of immunoglobulin variable region sequences, wherein a first immunoglobulin variable region sequence of the plurality has binding specificity for a first epitope and a second immunoglobulin variable region sequence of the plurality has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a multispecific antibody molecule comprises a third, fourth or fifth immunoglobulin variable region. In an embodiment, a multispecific antibody molecule is a bispecific antibody molecule, a trispecific antibody molecule, or a tetraspecific antibody molecule.

In an embodiment a multispecific antibody molecule is a bispecific antibody molecule. A bispecific antibody has specificity for no more than two antigens. A bispecific antibody molecule is characterized by a first immunoglobulin variable region sequence which has binding specificity for a first epitope and a second immunoglobulin variable region sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a bispecific antibody molecule comprises a heavy chain variable region sequence and a light chain variable region sequence which have binding specificity for a first epitope and a heavy chain variable region sequence and a light chain variable region sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv or a Fab, or fragment thereof, have binding specificity for a first epitope and a scFv or a Fab, or fragment thereof, have binding specificity for a second epitope.

In an embodiment, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable region sequence (abbreviated herein as VH), and a light (L) chain variable region sequence (abbreviated herein as VL). In an embodiment an antibody molecule comprises or consists of a heavy chain and a light chain (referred to herein as a half antibody. In another example, an antibody molecule includes two heavy (H) chain variable region sequences and two light (L) chain variable region sequence, thereby forming two antigen binding sites, such as Fab, Fab′, F(ab′)₂, Fc, Fd, Fd′, Fv, single chain antibodies (scFv for example), single variable region antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The a preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable region; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

Antibody molecules include intact molecules as well as functional fragments thereof. Constant regions of the antibody molecules can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable region derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable region (VH) are numbered 31-35 (HCDR1), 50-65 (HCDR2), and 95-102 (HCDR3); and the CDR amino acid residues in the light chain variable region (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3).

Each VH and VL typically includes three CDRs and four FRs, arranged from amino-terminus to carboxy-terminus in the following order: FR1, CDR1, FR2, CDR2, FR3, CDR3, FR4.

The antibody molecule can be a polyclonal or a monoclonal antibody. The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody molecule can be one in which the variable region, or a portion thereof, e.g., the CDRs, are generated in a non-human organism, e.g., a rat or mouse. Chimeric, CDR-grafted, and humanized antibodies are within the invention. Antibody molecules generated in a non-human organism, e.g., a rat or mouse, and then modified, e.g., in the variable framework or constant region, to decrease antigenicity in a human are within the invention.

An “effectively human” protein is a protein that does substantially not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding to the antigen. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody molecule can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibody molecules can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibody molecules in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

### Antibody Molecules Targeting CSF1R

In some embodiments, provided herein are antibody molecules (e.g., monospecific or multispecific antibody molecules) that bind to CSF1R, e.g., human CSF1R.

In some embodiments, the anti-CSF1R antibody molecule comprises one, two, or three heavy chain CDRs (e.g., HCDR1, HCDR2, and/or HCDR3) disclosed in Table 13, e.g., in one row of Table 13, or an amino acid sequence comprising no more than 1, 2, 3, 4, 5, or 6 modifications. In some embodiments, the anti-CSF1R antibody molecule comprises one, two, or three heavy chain CDRs (e.g., HCDR1, HCDR2, and/or HCDR3) from a heavy chain variable region (VH) disclosed in Table 15, e.g., optionally wherein the CDRs are according to the Kabat, Chothia, or IMGT definition. In some embodiments, the anti-CSF1R antibody molecule comprises one, two, three, or four heavy chain framework regions (e.g., heavy chain FR1, FR2, FR3, and/or FR4) disclosed in Table 13, e.g., in one row of Table 13, or an amino acid sequence comprising no more than 1, 2, 3, 4, 5, or 6 modifications. In some embodiments, the anti-CSF1R antibody molecule comprises one, two, three, or four heavy chain framework regions (e.g., heavy chain FR1, FR2, FR3, and/or FR4) from a VH disclosed in Table 15. In some embodiments, the anti-CSF1R antibody molecule comprises a VH disclosed in Table 15, or an amino acid sequence having at least 90, 91, 92, 93, 94, 95, 96, 97, 98, or 99% identity thereto.

In some embodiments, the anti-CSF1R antibody molecule comprises one, two, or three light chain CDRs (e.g., LCDR1, LCDR2, and/or LCDR3) disclosed in Table 14, e.g., in one row of Table 14, or an amino acid sequence comprising no more than 1, 2, 3, 4, 5, or 6 modifications. In some embodiments, the anti-CSF1R antibody molecule comprises one, two, or three light chain CDRs (e.g., LCDR1, LCDR2, and/or LCDR3) from a light chain variable region (VL) disclosed in Table 16, e.g., optionally wherein the CDRs are according to the Kabat, Chothia, or IMGT definition. In some embodiments, the anti-CSF1R antibody molecule comprises one, two, three, or four light chain framework regions (e.g., light chain FR1, FR2, FR3, and/or FR4) disclosed in Table 14, e.g., in one row of Table 14, or an amino acid sequence comprising no more than 1, 2, 3, 4, 5, or 6 modifications. In some embodiments, the anti-CSF1R antibody molecule comprises one, two, three, or four light chain framework regions (e.g., light chain FR1, FR2, FR3, and/or FR4) from a VL disclosed in Table 16. In some embodiments, the anti-CSF1R antibody molecule comprises a VL disclosed in Table 16, or an amino acid sequence having at least 90, 91, 92, 93, 94, 95, 96, 97, 98, or 99% identity thereto.

In some embodiments, the anti-CSF1R antibody molecule comprises a VH encoded by a nucleotide sequence disclosed in Table 15 (or a nucleotide sequence having at least 80, 85, 90, or 95% identity thereto), and/or a VL encoded by a nucleotide sequence disclosed in Table 16 (or a nucleotide sequence having at least 80, 85, 90, or 95% identity thereto).

TABLE 13
CDRs and framework regions (FR) of exemplary heavy chain
variable regions of anti-CSF1R antibody molecules.

| VH (SEQ | | | | | | | |
|---|---|---|---|---|---|---|---|
| ID NO) | FR 1 | HCDR 1 | FR 2 | HCDR 2 | FR 3 | HCDR 3 | FR 4 |
| aCSF1R | EVQLVES | GFTFSND | WIRQAP | YISSRSS | RFTISRDNAR | GVDGD | WGQGTL |
| BI117 VH | GGGLVQP | EMN | GKGLEW | TIYYADA | NSLYLQMNSL | FSFDY | VTVSS |
| (SEQ ID | GGSLRLS | (SEQ ID | VS | VKG | RAEDTAVYYC | (SEQ | (SEQ |
| NO: 323) | CAAS | NO: 402) | (SEQ | (SEQ ID | AR (SEQ ID | ID NO: | ID NO: |
| | (SEQ ID | | ID NO: | NO: 404) | NO: 428) | 413) | 430) |
| | NO: 424) | | 426) | | | | |
| aCSF1R | EVQLVES | GFTFSND | WIRQAP | YISSGSS | RFTISRDNAR | GVDGD | WGQGTL |
| BI117 C5- | GGGLVQP | EMN | GKGLEW | TIYYADA | NSLYLQMNSL | FSFDY | VTVSS |
| R2A VH | GGSLRLS | (SEQ ID | VS | VKG | RAEDTAVYYC | (SEQ | (SEQ |
| (SEQ ID | CAAS | NO: 402) | (SEQ | (SEQ ID | AR (SEQ ID | ID NO: | ID NO: |
| NO: 324) | (SEQ ID | | ID NO: | NO: 405) | NO: 428) | 413) | 430) |
| | NO: 424) | | 426) | | | | |
| aCSF1R | EVQLVES | GFTFSND | WIRQAP | FISGGSS | RFTISRDNAR | GVDGD | WGQGTL |
| BI117 C9- | GGGLVQP | EMN | GKGLEW | TIYYADA | NSLYLQMNSL | FSFDY | VTVSS |
| R2A VH | GGSLRLS | (SEQ ID | VS | VKG | RAEDTAVYYC | (SEQ | (SEQ |
| (SEQ ID | CAAS | NO: 402) | (SEQ | (SEQ ID | AR (SEQ ID | ID NO: | ID NO: |
| NO: 325) | (SEQ ID | | ID NO: | NO: 406) | NO: 428) | 413) | 430) |
| | NO: 424) | | 426) | | | | |
| aCSF1R | EVQLVES | GFTFSND | WIRQAP | YISSRSS | RFTISRDNAR | NSDED | WGQGTL |
| BI117 D1- | GGGLVQP | EMN | GKGLEW | TIYYADA | NSLYLQMNSL | GSFDY | VTVSS |
| R2A VH | GGSLRLS | (SEQ ID | VS | VKG | RAEDTAVYYC | (SEQ | (SEQ |
| (SEQ ID | CAAS | NO: 402) | (SEQ | (SEQ ID | AR (SEQ ID | ID NO: | ID NO: |
| NO: 326) | (SEQ ID) | | ID NO: | NO: 404) | NO: 428) | 414) | 430) |
| | NO: 424 | | 426) | | | | |
| aCSF1R | EVQLVES | GFTFSND | WIRQAP | YITSKSS | RFTISRDNAR | GVDGD | WGQGTL |
| BI117 E2- | GGGLVQP | EMN | GKGLEW | TIYYADS | NSLYLQMNSL | FSFDY | VTVSS |
| R2B VH | GGSLRLS | (SEQ ID | VS | VKG | RAEDTAVYYC | (SEQ | (SEQ |
| (SEQ ID | CAAS | NO: 402) | (SEQ | (SEQ ID | AR (SEQ ID | ID NO: | ID NO: |
| NO: 327) | (SEQ ID | | ID NO: | NO: 407) | NO: 428) | 413) | 430) |
| | NO: 424) | | 426) | | | | |
| aCSF1R | EVQLVES | GFTFSND | WIRQAP | YISSRSS | RFTISRDNAR | GVDGD | WGQGTL |
| BI117 G1- | GGGLVQP | EMN | GKGLEW | TIYYADA | NSLYLQMNSL | FSFDV | VTVSS |
| TUB VH | GGSLRLS | (SEQ ID | VS | VKG | RAEDTAVYYC | (SEQ | (SEQ |
| (SEQ ID | CAAS | NO: 402) | (SEQ | (SEQ ID | AR (SEQ ID | ID NO: | ID NO: |
| NO: 328) | (SEQ ID | | ID NO: | NO: 404) | NO: 428) | 415) | 430) |
| | NO: 424) | | 426) | | | | |
| aCSF1R | EVQLVES | GFTFSND | WIRQAP | YITSKSS | RFTISRDNAR | GVDGD | WGQGTL |
| BI117 G12- | GGGLVQP | EMN | GKGLEW | TIYYADA | NSLYLQMNSL | FSFDY | VTVSS |
| TUB VH | GGSLRLS | (SEQ ID | VS | VKG | RAEDTAVYYC | (SEQ | (SEQ |
| (SEQ ID | CAAS | NO: 402) | (SEQ | (SEQ ID | AR (SEQ ID | ID NO: | ID NO: |
| NO: 329) | (SEQ ID | | ID NO: | NO: 408) | NO: 428) | 413) | 430) |
| | NO: 424) | | 426) | | | | |
| aCSF1R | EVQLVES | GFTFSND | WIRQAP | YISSRSS | RFTISRDNAR | SVDGD | WGQGTL |
| BI117 D6- | GGGLVQP | EMN | GKGLEW | TIYYADA | NSLYLQMNSL | GSFDY | VTVSS |
| R3B VH | GGSLRLS | (SEQ ID | VS | VKG | RAEDTAVYYC | (SEQ | (SEQ |
| (SEQ ID | CAAS | NO: 402) | (SEQ | (SEQ ID | AR (SEQ ID | ID NO: | ID NO: |
| NO: 330) | (SEQ ID | | ID NO: | NO: 404) | NO: 428) | 416) | 430) |
| | NO: 424) | | 426) | | | | |
| aCSF1R | EVQLVES | GFTFSND | WIRQAP | YISSRSS | RFTISRDNAR | SSDGS | WGQGTL |
| BI117 A6- | GGGLVQP | EMN | GKGLEW | TIYYADA | NSLYLQMNSL | TSFDY | VTVSS |
| R3B VH | GGSLRLS | (SEQ ID | VS | VKG | RAEDTAVYYC | (SEQ | (SEQ |
| (SEQ ID | CAAS | NO: 402) | (SEQ | (SEQ ID | AR (SEQ ID | ID NO: | ID NO: |
| NO: 331) | (SEQ ID | | ID NO: | NO: 404) | NO: 428) | 417) | 430) |
| | NO: 424) | | 426) | | | | |
| aCSF1R | EVQLVES | GFTFSND | WIRQAP | YISSSSS | RFTISRDNAR | GVDGD | WGQGTL |
| BI117 A11- | GGGLVQP | EMN | GKGLEW | TIYYADA | NSLYLQMNSL | FSFDY | VTVSS |
| R3B VH | GGSLRLS | (SEQ ID | VS | VTG | RAEDTAVYYC | (SEQ | (SEQ |
| (SEQ ID | CAAS | NO: 402) | (SEQ | (SEQ ID | AR (SEQ ID | ID NO: | ID NO: |
| NO: 332) | (SEQ ID | | ID NO: | NO: 409) | NO: 428) | 413) | 430) |
| | NO: 424) | | 426) | | | | |
| aCSF1R | EVQLVES | GFTFSND | WIRQAP | YISSRSS | RFTISRDNAR | NSDSD | WGQGTL |
| BI117 B2- | GGGLVQP | EMN | GKGLEW | TIYYADA | NSLYLQMNSL | GSFDI | VTVSS |
| R3B VH | GGSLRLS | (SEQ ID | VS | VKG | RAEDTAVYYC | (SEQ | (SEQ |
| (SEQ ID | CAAS | NO: 402) | (SEQ | (SEQ ID | AR (SEQ ID | ID NO: | ID NO: |
| NO: 333) | (SEQ ID | | ID NO: | NO: 404) | NO: 428) | 418) | 430) |
| | NO: 424) | | 426) | | | | |
| aCSF1R | EVQLVES | GFTFSND | WIRQAP | YISSRSS | RFTISRDNAR | GVDGD | WGQGTL |
| BI117 C5- | GGGLVQP | EMN | GKGLEW | TIYYADA | NSLYLQMNSL | FSFDF | VTVSS |
| R3B VH | GGSLRLS | (SEQ ID | VS | VKG | RAEDTAVYYC | (SEQ | (SEQ |
| (SEQ ID | CAAS | NO: 402) | (SEQ | (SEQ ID | AR (SEQ ID | ID NO: | ID NO: |
| NO: 334) | (SEQ ID | | ID NO: | NO: 404) | NO: 428) | 419) | 430) |
| | NO: 424) | | 426) | | | | |
| aCSF1R | EVQLVES | GFTFSND | WIRQAP | YISSRSS | RFTISRDNAR | SGRLD | WGQGTL |
| BI117 C6- | GGGLVQP | EMN | GKGLEW | TIYYADA | NSLYLQMNSL | GSFDY | VTVSS |
| R3B VH | GGSLRLS | (SEQ ID | VS | VKG | RAEDTAVYYC | (SEQ | (SEQ |
| (SEQ ID | CAAS | NO: 402) | (SEQ | (SEQ ID | AR (SEQ ID | ID NO: | ID NO: |
| NO: 335) | (SEQ ID | | ID NO: | NO: 404) | NO: 428) | 420) | 430) |
| | NO: 424) | | 426) | | | | |
| aCSF1R | EVQLVES | GFTFSND | WIRQAP | YISSRSS | RFTISRDNAR | GVESD | WGQGTL |
| BI117 D11- | GGGLVQP | EMN | GKGLEW | TIYYADA | NSLYLQMNSL | FSFDY | VTVSS |
| R3B VH | GGSLRLS | (SEQ ID | VS | VKG | RAEDTAVYYC | (SEQ | (SEQ |
| (SEQ ID | CAAS | NO: 402) | (SEQ | (SEQ ID | AR (SEQ ID | ID NO: | ID NO: |
| NO: 336) | (SEQ ID | | ID NO: | NO: 404) | NO: 428) | 421) | 430) |
| | NO: 424) | | 426) | | | | |
| aCSF1R | QVQLVQS | GFTFSSY | WVRQAP | VISYDGS | RFTISRDNSK | ELDYG | WGKGTT |
| BI123 VH | GGGVVQP | AMB | GKGLEW | NKYYADS | NTLYLQMNSL | DYGLD | VTVSS |
| (SEQ ID | GRSLRLS | (SEQ ID | VA | VKG | RAEDTAVYYC | V(SEQ | (SEQ |
| NO: 337) | CAAS | NO: 403) | (SEQ | (SEQ ID | AR (SEQ ID | IDNO: | ID NO: |
| | (SEQ ID | | ID NO: | NO: 410) | NO: 429) | 422) | 431) |
| | NO: 425) | | 427) | | | | |
| aCSF1R | QVQLVQS | GFTFSSY | WVRQAP | VISYDGS | RFTISRDNSK | ELDYG | WGKGTT |
| BI123 A5- | GGGVVQP | AMH | GKGLEW | NKYYADS | NTLYLQMNSL | DWGLD | VTVSS |
| R3B VH | GRSLRLS | (SEQ ID | VA | VKG | RAEDTAVYYC | V | (SEQ |
| (SEQ ID | CAAS | NO: 403) | (SEQ | (SEQ ID | AR (SEQ ID | (SEQ | ID NO: |
| NO: 338) | (SEQ ID | | ID NO: | NO: 410) | NO: 429) | ID NO: | 431) |
| | NO: 425) | | 427) | | | 423) | |
| aCSF1R | QVQLVQS | GFTFSSY | WVRQAP | VIPYYGS | RFTISRDNSK | ELDYG | WGKGTT |
| BI123 B6- | GGGVVQP | AMH | GKGLEW | NKYYADS | NTLYLQMNSL | DYGLD | VTVSS |
| R3B VH | GRSLRLS | (SEQ ID | VA | VKG | RAEDTAVYYC | V | (SEQ |
| (SEQ ID | CAAS | NO: 403) | (SEQ | (SEQ ID | AR (SEQ ID | (SEQ | ID NO: |
| NO: 339) | (SEQ ID | | ID NO: | NO: 411) | NO: 429) | ID NO: | 431) |
| | NO: 425) | | 427) | | | 422) | |
| aCSF1R | QVQLVQS | GFTFSSY | WVRQAP | VISYDGS | RFTISRDNSK | ELDYG | WGKGTT |
| BI123 C1- | GGGVVQP | AMH | GKGLEW | DKYYADS | NTLYLQMNSL | DYGLD | VTVSS |
| R3B VH | GRSLRLS | (SEQ ID | VA | VKG | RAEDTAVYYC | V | (SEQ |
| (SEQ ID | CAAS | NO: 403) | (SEQ | (SEQ ID | AR (SEQ ID | (SEQ | ID NO: |
| NO: 340) | (SEQ ID | | ID NO: | NO: 412) | NO: 429) | ID NO: | 431) |
| | NO: 425) | | 427) | | | 422) | |

TABLE 29
Consensus CDRs of exemplary heavy chain variable regions of
anti-CSF1R antibody molecules.

| Description | HCDR 1 | HCDR 2 | HCDR 3 |
|---|---|---|---|
| BI117 | GFTFSNDE | X₁IX₂X₃X₄₅STIYYADX₅VX₆G, | X₁X₂X₃X₄X₅X₆SFDX₇, wherein |
| consensus | MN (SEQ | wherein X₁ is Y or F; X₂ | X₁ is G, N, or S; X₂ is V, S, or |
| CDRs #1 | ID NO: | is S or T; X₃ is S or G; X₄ is | G; X₃ is D, E, or R; X₄ is G, S, |
| | 402) | R, K, G, or S; X₅ is A or S; | E, or L; X₅ is D or S; X₆ is F, |
| | | and X₆ is K or T (SEQ ID | G, or T; and X₇ is Y, V, F, or I |
| | | NO: 474) | (SEQ ID NO: 475) |
| BI117 | GFTFSNDE | X₁ISX₂X₃SSTIYYADAVKG, | X₁X₂DX₃DX₄SFDY, wherein X₁ |
| consensus | MN (SEQ | wherein X₁ is Y or F; X₂ is S | is G or N; X₂ is V or S; X₃ is G |
| CDRs #2 | ID NO: | or G; and X₃ is G or R (SEQ | or E; and X₄ is F or G (SEQ ID |
| | 402) | ID NO: 476) | NO: 477) |
| BI123 | GFTFSSYA | VIX₁YX₂GSX₃KYYADSVK | ELDYGDXGLDV, wherein X is Y or W |
| consensus | MH (SEQ | G, wherein X₁ is S or P; X₂ is | (SEQ ID NO: SEQ ID NO: 479) |
| CDRs | ID NO: | D or Y; and X₃ is N or D | |
| | 403) | (SEQ ID NO: 478) | |

TABLE 14
CDRs and framework regions (FR) of exemplary light chain
variable regions of anti-CSF1R antibody molecules.

| VL | FR 1 | LCDR 1 | FR 2 | LCDR 2 | FR 3 | LCDR 3 | FR 4 |
|---|---|---|---|---|---|---|---|
| aCSF1R | QLVLTQPPS | TGSSSNIG | WYQQLPG | GNSNRPS | GVPDRFSGSK | QSYDSS | FGGGTK |
| BI117 VL | VSGAPGQRV | AGYDVH | TAPKLLI | (SEQ | SGTSASLAIT | LSEEV | LTVL |
| (SEQ ID | TISC (SEQ | (SEQ ID | Y (SEQ | ID NO: | GLQAEDEADY | (SEQ | (SEQ |
| NO: 341) | ID NO: | NO: 432) | ID NO: | 434) | YC (SEQ ID | ID NO: | ID NO: |
| | 438) | | 440) | | NO: 442) | 436) | 444) |
| aCSF1R | SLVLTQPPS | GGNNIGSK | WYQQKPG | DDSDRPS | GIPERFSGSN | QVWDSS | FGGGTQ |
| BI123 VL | VSVAPGKTA | SVH (SEQ | QAPVLVV | (SEQ | SGNTATLTIS | SDHVV | LTVL |
| (SEQ ID | RITC (SEQ | ID NO: | Y (SEQ | ID NO: | RVEAGDEADY | (SEQ | (SEQ |
| NO: 342) | ID NO: | 433) | ID NO: | 435) | YC (SEQ ID | ID NO: | ID NO: |
| | 439) | | 441) | | NO: 443) | 437) | 445) |

TABLE 15
Amino acid and nucleotide sequences of exemplary heavy chain
variable regions of anti-CSF1R antibody molecules.

| SEQ ID NO | Description | Sequence |
|---|---|---|
| SEQ ID NO: | aCSF1R | EVQLVESGGGLVQPGGSLRLSCAASGFTFSNDEMNWIRQAPGKGLEWVS |
| 323 | BI117 VH | YISSRSSTIYYADAVKGRFTISRDNARNSLYLQMNSLRAEDTAVYYCAR |
| | aa | GVDGDFSFDYWGQGTLVTVSS |
| SEQ ID NO: | aCSF1R | EVQLVESGGGLVQPGGSLRLSCAASGFTFSNDEMNWIRQAPGKGLEWVS |
| 324 | BI117 C5- | YISSGSSTIYYADAVKGRFTISRDNARNSLYLQMNSLRAEDTAVYYCAR |
| | R2A VH aa | GVDGDFSFDYWGQGTLVTVSS |
| SEQ ID NO: | aCSF1R | EVQLVESGGGLVQPGGSLRLSCAASGFTFSNDEMNWIRQAPGKGLEWVS |
| 325 | BI117 C9- | FISGGSSTIYYADAVKGRFTISRDNARNSLYLQMNSLRAEDTAVYYCAR |
| | R2A VH aa | GVDGDFSFDYWGQGTLVTVSS |
| SEQ ID NO: | aCSF1R | EVQLVESGGGLVQPGGSLRLSCAASGFTFSNDEMNWIRQAPGKGLEWVS |
| 326 | BI117 D1- | YISSRSSTIYYADAVKGRFTISRDNARNSLYLQMNSLRAEDTAVYYCAR |
| | R2A VH aa | NSDEDGSFDYWGQGTLVTVSS |
| SEQ ID NO: | aCSF1R | EVQLVESGGGLVQPGGSLRLSCAASGFTFSNDEMNWIRQAPGKGLEWVS |
| 327 | BI117 E2- | YITSKSSTIYYADSVKGRFTISRDNARNSLYLQMNSLRAEDTAVYYCAR |
| | R2B VH aa | GVDGDFSFDYWGQGTLVTVSS |
| SEQ ID NO: | aCSF1R | EVQLVESGGGLVQPGGSLRLSCAASGFTFSNDEMNWIRQAPGKGLEWVS |
| 328 | BI117 G1- | YISSRSSTIYYADAVKGRFTISRDNARNSLYLQMNSLRAEDTAVYYCAR |
| | R2B VH aa | GVDGDFSFDVWGQGTLVTVSS |
| SEQ ID NO: | aCSF1R | EVQLVESGGGLVQPGGSLRLSCAASGFTFSNDEMNWIRQAPGKGLEWVS |
| 329 | BI117 G12- | YITSKSSTIYYADAVKGRFTISRDNARNSLYLQMNSLRAEDTAVYYCAR |
| | R2B VH aa | GVDGDFSFDYWGQGTLVTVSS |
| SEQ ID NO: | aCSF1R | EVQLVESGGGLVQPGGSLRLSCAASGFTFSNDEMNWIRQAPGKGLEWVS |
| 330 | BI117 D6- | YISSRSSTIYYADAVKGRFTISRDNARNSLYLQMNSLRAEDTAVYYCAR |
| | R3B VH aa | SVDGDGSFDYWGQGTLVTVSS |
| SEQ ID NO: | aCSF1R | EVQLVESGGGLVQPGGSLRLSCAASGFTFSNDEMNWIRQAPGKGLEWVS |
| 331 | BI117 A6- | YISSRSSTIYYADAVKGRFTISRDNARNSLYLQMNSLRAEDTAVYYCAR |
| | R3B VH aa | SSDGSTSFDYWGQGTLVTVSS |
| SEQ ID NO: | aCSF1R | EVQLVESGGGLVQPGGSLRLSCAASGFTFSNDEMNWIRQAPGKGLEWVS |
| 332 | BI117 A11- | YISSSSSTIYYADAVTGRFTISRDNARNSLYLQMNSLRAEDTAVYYCAR |
| | R3B VH aa | GVDGDFSFDYWGQGTLVTVSS |
| SEQ ID NO: | aCSF1R | EVQLVESGGGLVQPGGSLRLSCAASGFTFSNDEMNWIRQAPGKGLEWVS |
| 333 | BI117 B2- | YISSRSSTIYYADAVKGRFTISRDNARNSLYLQMNSLRAEDTAVYYCAR |
| | R3B VH aa | NSDSDGSFDIWGQGTLVTVSS |
| SEQ ID NO: | aCSF1R | EVQLVESGGGLVQPGGSLRLSCAASGFTFSNDEMNWIRQAPGKGLEWVS |
| 334 | BI117 C5- | YISSRSSTIYYADAVKGRFTISRDNARNSLYLQMNSLRAEDTAVYYCAR |
| | R3B VH aa | GVDGDFSFDFWGQGTLVTVSS |
| SEQ ID NO: | aCSF1R | EVQLVESGGGLVQPGGSLRLSCAASGFTFSNDEMNWIRQAPGKGLEWVS |
| 335 | BI117 C6- | YISSRSSTIYYADAVKGRFTISRDNARNSLYLQMNSLRAEDTAVYYCAR |
| | R3B VH aa | SGRLDGSFDYWGQGTLVTVSS |
| SEQ ID NO: | aCSF1R | EVQLVESGGGLVQPGGSLRLSCAASGFTFSNDEMNWIRQAPGKGLEWVS |
| 336 | BI117 D11- | YISSRSSTIYYADAVKGRFTISRDNARNSLYLQMNSLRAEDTAVYYCAR |
| | R3B VH aa | GVESDFSFDYWGQGTLVTVSS |
| SEQ ID NO: | aCSF1R | QVQLVQSGGGVVQPGRSLRLSCAASGFTFSSYAMHWVRQAPGKGLEWVA |
| 337 | BI123 VH | VISYDGSNKYYADSVKGRFTISRDNSKNTLYLQMNSLRAEDTAVYYCAR |
| | aa | ELDYGDYGLDVWGKGTTVTVSS |
| SEQ ID NO: | aCSF1R | QVQLVQSGGGVVQPGRSLRLSCAASGFTFSSYAMHWVRQAPGKGLEWVA |
| 338 | BI123 A5- | VISYDGSNKYYADSVKGRFTISRDNSKNTLYLQMNSLRAEDTAVYYCAR |
| | R3B VH aa | ELDYGDWGLDVWGKGTTVTVSS |
| SEQ ID NO: | aCSF1R | QVQLVQSGGGVVQPGRSLRLSCAASGFTFSSYAMHWVRQAPGKGLEWVA |
| 339 | BI123 B6- | VIPYYGSNKYYADSVKGRFTISRDNSKNTLYLQMNSLRAEDTAVYYCAR |
| | R3B VH aa | ELDYGDYGLDVWGKGTTVTVSS |
| SEQ ID NO: | aCSF1R | QVQLVQSGGGVVQPGRSLRLSCAASGFTFSSYAMHWVRQAPGKGLEWVA |
| 340 | BI123 C1- | VISYDGSDKYYADSVKGRFTISRDNSKNTLYLQMNSLRAEDTAVYYCAR |
| | R3B VH aa | ELDYGDYGLDVWGKGTTVTVSS |
| SEQ ID NO: | aCSF1R | GAAGTGCAGCTGGTCGAAAGCGGAGGGGGTCTGGTCCAGCCTGGAGGGT |
| 250 | BI117 VH | CCCTGCGGCTGTCCTGCGCCGCTTCCGGCTTCACCTTCTCCAACGATGA |
| | nt | AATGAACTGGATCCGCCAAGCCCCTGGAAAGGGCCTGGAATGGGTGTCG |
| | | TACATCTCGAGCAGGTCCAGCACTATCTACTATGCCGACGCGGTGAAGG |
| | | GACGCTTCACTATCAGCCGCGATAACGCACGCAATTCCCTCTACCTGCA |
| | | GATGAACTCGCTCAGAGCTGAGGATACTGCCGTCTACTACTGCGCCCGC |
| | | GGCGTGGACGGCGATTTTTCCTTCGATTACTGGGGACAGGGCACTCTTG |
| | | TCACCGTGTCAAGC |
| SEQ ID NO: | aCSF1R | GAAGTCCAACTCGTGGAAAGCGGCGGTGGCCTGGTGCAGCCGGGAGGCT |
| 251 | BI117 C5- | CCCTCCGGCTGTCGTGTGCAGCCAGCGGGTTCACCTTCTCCAACGACGA |
| | R2A VH nt | AATGAACTGGATTCGCCAGGCGCCGGGAAAGGGGCTGGAATGGGTGTCG |
| | | TACATTTCAAGCGGGTCCTCCACCATTTACTACGCCGATGCAGTCAAGG |
| | | GCCGCTTCACCATCTCGCGCGACAACGCTCGGAACTCCCTCTATCTGCA |
| | | GATGAACAGCCTGAGAGCGGAAGACACCGCCGTCTACTACTGCGCCCGC |
| | | GGAGTGGACGGCGACTTCTCCTTCGACTACTGGGGTCAGGGAACCCTGG |
| | | TGACTGTGTCCTCA |
| SEQ ID NO: | aCSF1R | GAGGTGCAGCTGGTGGAATCGGGGGGTGGCCTCGTGCAGCCCGGAGGGT |
| 252 | BI117 C9- | CGCTCCGGCTGTCCTGTGCCGCTAGCGGATTCACATTCTCGAATGACGA |
| | R2A VH nt | GATGAACTGGATTCGGCAGGCCCCAGGCAAGGGCCTGGAATGGGTGAGC |
| | | TTCATTTCGGGCGGGTCCTCAACCATCTACTACGCCGACGCCGTCAAGG |
| | | GGCGCTTTACTATCTCCCGCGACAATGCACGGAACTCCCTCTACCTGCA |
| | | AATGAACAGCCTGAGGGCGGAAGATACAGCTGTGTACTACTGCGCCCGG |
| | | GGCGTGGACGGAGACTTCTCCTTCGACTACTGGGGTCAGGGAACCCTGG |
| | | TGACAGTGTCTTCC |
| SEQ ID NO: | aCSF1R | GAAGTGCAGCTCGTGGAGTCTGGAGGTGGTCTGGTGCAGCCGGGTGGTT |
| 253 | BI117 D1- | CCCTGAGGCTGTCCTGCGCCGCCAGCGGCTTCACCTTCTCAAATGACGA |
| | R2A VH nt | AATGAACTGGATTCGGCAGGCCCCTGGCAAGGGCCTGGAGTGGGTGTCG |
| | | TACATCTCCTCCCGCAGCTCCACCATCTACTACGCCGACGCAGTCAAGG |
| | | GGAGGTTCACCATCTCCCGGGACAACGCCCGGAACTCACTGTACCTGCA |
| | | GATGAACTCCCTGAGAGCTGAGGACACTGCCGTCTACTACTGCGCTAGA |
| | | AACTCCGATGAGGACGGAAGCTTCGACTACTGGGGCCAGGGAACTCTGG |
| | | TGACAGTCTCGAGC |
| SEQ ID NO: | aCSF1R | GAGGTGCAGCTCGTGGAAAGCGGGGGCGGCCTGGTCCAGCCTGGAGGAT |
| 254 | BI117 E2- | CCCTGAGACTTTCCTGTGCTGCTTCCGGGTTCACCTTCAGCAATGATGA |
| | R2B VH nt | GATGAACTGGATTCGGCAAGCCCCGGGCAAGGGCCTGGAATGGGTCAGC |
| | | TACATCACGTCTAAGTCCTCCACCATCTACTATGCCGACTCCGTGAAGG |
| | | GACGCTTTACCATCTCACGCGACAACGCGCGGAACAGCCTTTACCTGCA |
| | | GATGAACTCACTGCGCGCCGAAGATACCGCCGTGTACTACTGCGCTCGC |
| | | GGAGTGGATGGAGATTTTTCCTTCGATTACTGGGGACAAGGCACCCTGG |
| | | TGACCGTCAGCTCC |
| SEQ ID NO: | aCSF1R | GAAGTCCAGCTCGTCGAATCTGGTGGGGGACTGGTCCAGCCGGGAGGAT |
| 255 | BI117 G1- | CCCTCCGGCTGTCGTGCGCGGCATCCGGGTTCACCTTCTCCAACGATGA |
| | R23 VH nt | GATGAATTGGATTCGCCAGGCCCCAGGAAAGGGGCTGGAGTGGGTCTCG |
| | | TACATCTCATCCCGCAGCTCTACTATCTACTACGCCGATGCTGTCAAGG |
| | | GCCGGTTCACTATCTCCCGCGATAACGCCCGGAACTCCCTCTACCTCCA |
| | | GATGAACTCACTGCGCGCGGAAGACACCGCCGTCTACTACTGCGCACGG |
| | | GGCGTCGATGGAGATTTCTCCTTCGATGTGTGGGGTCAGGGAACACTGG |
| | | TGACCGTGTCCTCC |
| SEQ ID NO: | aCSF1R | GAGGTGCAGCTCGTGGAGTCCGGTGGAGGACTGGTGCAGCCCGGCGGAT |
| 256 | BI117 G12- | CGCTGAGACTGTCTTGCGCCGCCTCGGGATTCACGTTCTCCAACGATGA |
| | R23 VH nt | AATGAACTGGATTAGACAGGCTCCGGGCAAGGGCCTGGAATGGGTGTCC |
| | | TACATTACTTCGAAGTCCTCCACCATTTACTACGCAGACGCCGTGAAGG |
| | | GCCGCTTCACCATTTCCCGCGACAACGCAAGAAACTCGCTGTACCTGCA |
| | | GATGAACTCCCTGCGCGCGGAGGACACCGCCGTGTACTACTGTGCCAGA |
| | | GGAGTCGATGGCGATTTCTCGTTCGACTACTGGGGACAGGGCACTCTTG |
| | | TGACTGTCTCCTCC |
| SEQ ID NO: | aCSF1R | GAGGTCCAGCTGGTCGAATCGGGGGGAGGCCTCGTGCAGCCTGGAGGGT |
| 257 | BI117 D6- | CCCTCCGGCTTTCCTGTGCCGCTTCCGGATTCACCTTCAGCAACGATGA |
| | R3B VH nt | GATGAACTGGATCAGGCAGGCCCCGGGAAAGGGGCTCGAGTGGGTGTCC |
| | | TACATCTCCAGCCGGTCCTCGACCATCTACTACGCCGATGCCGTGAAGG |
| | | GGAGATTTACTATCTCCCGCGACAATGCACGGAACTCACTGTACCTCCA |
| | | GATGAACTCACTGCGGGCCGAGGACACCGCAGTGTACTACTGCGCGCGG |
| | | TCCGTGGATGGCGACGGGAGCTTCGATTACTGGGGACAGGGGACCCTGG |
| | | TGACCGTGTCGTCC |
| SEQ ID NO: | aCSF1R | GAGGTGCAGCTCGTGGAATCCGGAGGTGGACTGGTGCAGCCGGGGGGTA |
| 258 | BI117 A6- | GCCTGCGGCTGTCCTGTGCAGCTTCCGGGTTCACCTTCTCCAACGACGA |
| | R3B VH nt | AATGAACTGGATCCGCCAGGCCCCGGGCAAGGGACTCGAATGGGTGTCC |
| | | TACATCTCCTCCCGCTCCTCCACTATCTACTATGCCGATGCCGTCAAGG |
| | | GCCGCTTCACCATTTCTCGCGACAACGCCCGGAACTCCCTGTACCTCCA |
| | | GATGAACTCCCTGCGCGCCGAGGACACTGCCGTCTACTACTGCGCGAGG |
| | | TCGTCCGACGGATCCACCTCCTTCGACTACTGGGGGCAAGGAACTCTCG |
| | | TGACCGTGTCCTCC |
| SEQ ID NO: | aCSF1R | GAAGTGCAACTTGTCGAAAGTGGTGGGGGCCTGGTGCAGCCTGGGGGCT |
| 259 | BI117 A11- | CCCTGCGGCTGTCCTGCGCCGCCTCCGGATTCACTTTCTCCAACGACGA |
| | R3B VH nt | GATGAACTGGATTAGACAGGCTCCTGGAAAGGGTCTCGAATGGGTGTCC |
| | | TACATTAGCTCATCTTCCAGCACCATCTACTACGCCGACGCAGTGACCG |
| | | GAAGGTTCACCATCTCGAGGGACAACGCCCGCAACTCCCTCTACCTGCA |
| | | GATGAACTCGCTGCGCGCGGAGGACACCGCTGTGTACTACTGCGCCCGC |
| | | GGCGTGGACGGAGACTTCTCCTTCGACTACTGGGGACAGGGAACTCTTG |
| | | TGACCGTGAGCAGC |
| SEQ ID NO: | aCSF1R | GAAGTGCAACTGGTGGAATCGGGAGGCGGCCTGGTGCAACCTGGCGGTA |
| 260 | BI117 B2- | GCCTGCGGCTGAGCTGCGCCGCCTCAGGCTTCACCTTTTCCAACGACGA |
| | R3B VH nt | AATGAACTGGATCAGACAGGCGCCTGGCAAAGGACTGGAGTGGGTGTCC |
| | | TACATCTCGTCCAGGTCAAGCACCATTTATTACGCGGACGCCGTGAAAG |
| | | GGAGATTCACCATCAGCAGAGACAACGCCCGGAACTCCCTCTACCTGCA |
| | | GATGAACTCCCTGAGGGCCGAGGACACCGCAGTGTACTACTGTGCCAGA |
| | | AACTCCGACTCCGATGGAAGCTTCGACATCTGGGGACAGGGGACCCTGG |
| | | TGACTGTGTCGTCC |
| SEQ ID NO: | aCSF1R | GAAGTGCAGCTGGTCGAATCGGGGGGAGGACTGGTCCAGCCTGGAGGGT |
| 261 | BI117 C5- | CCCTCCGCCTGAGCTGCGCCGCCTCCGGATTCACCTTTTCCAACGACGA |
| | R3B VH nt | GATGAACTGGATCAGGCAGGCCCCGGGCAAGGGGCTGGAATGGGTGTCA |
| | | TACATCTCCAGCCGCTCATCGACAATTTACTACGCCGATGCCGTGAAGG |
| | | GTAGATTTACCATCTCCCGGGACAATGCCCGAAACAGCCTGTATCTGCA |
| | | AATGAACTCCCTGCGGGCGGAGGATACCGCTGTCTACTACTGTGCCAGA |
| | | GGCGTGGATGGGGACTTCAGCTTTGACTTTTGGGGACAGGGCACCCTCG |
| | | TCACCGTGAGCAGC |
| SEQ ID NO: | aCSF1R | GAGGTGCAGCTGGTGGAGTCCGGAGGCGGCCTTGTCCAGCCGGGCGGGT |
| 262 | BI117 C6- | CTCTGCGGCTGTCATGTGCGGCCTCGGGATTCACTTTTTCCAACGACGA |
| | R3B VH nt | AATGAACTGGATCCGCCAGGCCCCTGGGAAGGGGCTTGAATGGGTGAGC |
| | | TACATCTCCTCACGGTCCTCCACTATCTATTACGCCGACGCTGTGAAGG |
| | | GCCGCTTCACTATCTCACGCGACAATGCCAGGAACTCTCTCTACCTGCA |
| | | GATGAACTCCCTGCGGGCCGAAGATACCGCGGTGTACTACTGTGCCCGC |
| | | TCGGGCCGGCTGGACGGGTCATTCGACTACTGGGGACAAGGGACCCTGG |
| | | TCACCGTCTCCTCT |
| SEQ ID NO: | aCSF1R | GAAGTCCAGCTGGTCGAGTCCGGCGGTGGGCTGGTCCAGCCCGGCGGAT |
| 263 | BI117 D11- | CGCTGCGCCTGTCCTGTGCCGCCTCCGGCTTCACCTTCTCCAACGACGA |
| | R3B VH nt | GATGAACTGGATCAGACAGGCCCCCGGTAAGGGACTGGAGTGGGTGAGC |
| | | TACATCTCCTCGCGCAGCTCCACCATCTACTACGCCGACGCCGTGAAGG |
| | | GAAGGTTCACCATCAGCAGAGATAACGCGCGGAACAGCCTGTACCTGCA |
| | | GATGAACTCACTGCGGGCTGAAGACACAGCCGTCTATTACTGTGCCAGA |
| | | GGAGTGGAATCCGATTTCAGCTTCGACTACTGGGGACAAGGAACTCTGG |
| | | TCACGGTCAGCTCC |
| SEQ ID NO: | aCSF1R | CAGGTGCAGCTTGTGCAAAGCGGGGGAGGCGTGGTGCAGCCGGGACGGA |
| 264 | BI123 VH | GCCTGCGGCTGTCGTGCGCCGCGAGCGGTTTCACCTTCTCCTCCTACGC |
| | nt | TATGCACTGGGTCCGCCAGGCGCCGGGCAAGGGCCTCGAATGGGTGGCC |
| | | GTGATCAGCTACGATGGCTCCAACAAGTACTACGCCGACTCAGTGAAGG |
| | | GAAGGTTCACCATCTCAAGAGATAACAGCAAGAACACGCTGTACCTCCA |
| | | GATGAACTCCCTCCGGGCCGAAGACACAGCCGTCTACTACTGTGCGAGA |
| | | GAGCTCGATTACGGCGATTACGGCCTGGACGTGTGGGGCAAGGGAACCA |
| | | CGGTGACCGTCTCCTCC |
| SEQ ID NO: | aCSF1R | CAAGTGCAGCTCGTGCAGTCAGGCGGTGGCGTGGTGCAGCCCGGCCGGT |
| 265 | BI123 A5- | CTCTGAGGCTCAGCTGCGCCGCCTCGGGCTTCACCTTCTCGTCATACGC |
| | R3B VH nt | CATGCACTGGGTGCGCCAGGCCCCCGGGAAGGGACTGGAATGGGTCGCC |
| | | GTGATCTCCTACGATGGATCGAACAAGTACTACGCCGACTCCGTGAAGG |
| | | GGCGGTTCACCATCTCCCGCGACAACTCCAAGAACACCCTTTATCTGCA |
| | | AATGAACTCGCTCAGAGCAGAGGACACCGCTGTGTACTACTGCGCCAGA |
| | | GAGCTGGACTACGGTGATTGGGGACTGGACGTGTGGGGCAAGGGTACCA |
| | | CCGTGACTGTGTCGTCC |
| SEQ ID NO: | aCSF1R | CAAGTGCAACTGGTGCAATCCGGAGGGGGTGTGGTCCAACCGGGACGCA |
| 266 | BI123 B6- | GCCTGCGGCTGTCCTGCGCCGCCAGCGGCTTCACATTCTCCTCCTACGC |
| | R3B VH nt | CATGCACTGGGTCCGCCAGGCGCCAGGTAAGGGACTCGAATGGGTGGCA |
| | | GTGATCCCTTACTACGGATCAAACAAGTATTACGCCGATTCCGTGAAGG |
| | | GCCGCTTCACCATCAGCAGAGACAACTCCAAGAACACCCTCTACCTGCA |
| | | GATGAACTCCCTTCGCGCCGAGGATACCGCCGTCTACTACTGCGCCCGC |
| | | GAGCTGGACTACGGTGACTACGGACTCGATGTGTGGGGAAAGGGTACCA |
| | | CTGTGACCGTGAGCTCC |
| SEQ ID NO: | aCSF1R | CAGGTGCAACTGGTCCAGTCGGGCGGAGGAGTGGTCCAGCCGGGTAGGT |
| 267 | BI123 C1- | CACTGCGGCTGTCATGTGCCGCTAGCGGATTCACTTTCTCTTCCTACGC |
| | R3B VH nt | AATGCACTGGGTGAGACAAGCCCCTGGCAAGGGACTGGAGTGGGTCGCC |
| | | GTGATCTCGTATGACGGGTCCGATAAGTACTACGCTGATTCCGTGAAGG |
| | | GACGCTTCACGATCAGCCGCGACAACTCCAAAAACACTCTCTACCTGCA |
| | | AATGAATTCCCTTCGCGCCGAAGATACTGCAGTGTACTACTGCGCCAGA |
| | | GAGCTGGACTACGGAGACTACGGCCTGGACGTGTGGGGCAAGGGCACCA |
| | | CTGTGACCGTCTCCTCC |

TABLE 16
Amino acid and nucleotide sequences of exemplary light
chain variable regions of anti-CSF1R antibody molecules.

| SEQ ID NO | Description | Sequence |
|---|---|---|
| SEQ ID NO: | aCSF1R BI117 | QLVLTQPPSVSGAPGQRVTISCTGSSSNIGAGYDVHWYQ |
| 341 | VL aa | QLPGTAPKLLIYGNSNRPSGVPDRFSGSKSGTSASLAIT |
| | | GLQAEDEADYYCQSYDSSLSEEVFGGGTKLTVL |
| SEQ ID NO: | aCSF1R BI123 | SLVLTQPPSVSVAPGKTARITCGGNNIGSKSVHWYQQKP |
| 342 | VL aa | GQAPVLVVYDDSDRPSGIPERFSGSNSGNTATLTISRVE |
| | | AGDEADYYCQVWDSSSDHVVFGGGTQLTVL |
| SEQ ID NO: | aCSF1R BI117 | CAACTGGTGCTGACCCAGCCCCCTTCCGTCTCCGGCGCC |
| 268 | VL nt | CCGGGCCAGCGCGTCACTATCTCCTGTACCGGCAGCTCC |
| | | AGCAACATCGGAGCAGGATACGACGTGCACTGGTACCAA |
| | | CAGCTCCCCGGAACCGCGCCTAAGCTGCTGATCTACGGG |
| | | AACTCTAACCGGCCGTCCGGCGTGCCTGACCGCTTCAGC |
| | | GGATCGAAGTCCGGCACCAGCGCCTCCCTGGCCATCACT |
| | | GGACTGCAGGCCGAGGACGAAGCCGATTACTACTGCCAG |
| | | TCCTACGACTCCTCCCTGTCCGAGGAAGTGTTCGGAGGC |
| | | GGGACGAAGCTCACCGTCCTG |
| SEQ ID NO: | aCSF1R BI123 | TCCCTCGTGCTGACCCAACCTCCTTCAGTCTCCGTGGCC |
| 269 | VL nt | CCCGGCAAGACCGCCCGCATCACCTGTGGCGGTAACAAC |
| | | ATCGGCTCCAAGTCAGTCCACTGGTACCAGCAGAAGCCG |
| | | GGCCAAGCTCCCGTGCTGGTCGTGTACGACGACTCAGAC |
| | | CGCCCCAGCGGTATCCCTGAGAGATTTAGCGGATCCAAC |
| | | TCCGGGAACACCGCTACCCTGACCATCTCAAGGGTGGAG |
| | | GCCGGAGACGAAGCCGACTACTACTGCCAGGTGTGGGAT |
| | | TCCAGCTCCGACCACGTGGTGTTCGGCGGCGGCACTCAG |
| | | CTCACCGTGCTG |

### Antibody Molecules Targeting CCR2

In some embodiments, provided herein are antibody molecules (e.g., monospecific or multispecific antibody molecules) that bind to CCR2, e.g., human CCR2.

In some embodiments, the anti-CCR2 antibody molecule comprises one, two, or three heavy chain CDRs (e.g., HCDR1, HCDR2, and/or HCDR3) disclosed in Table 17, e.g., in one row of Table 17, or an amino acid sequence comprising no more than 1, 2, 3, 4, 5, or 6 modifications. In some embodiments, the anti-CCR2 antibody molecule comprises one, two, or three heavy chain CDRs (e.g., HCDR1, HCDR2, and/or HCDR3) from a heavy chain variable region (VH) disclosed in Table 19, e.g., optionally wherein the CDRs are according to the Kabat, Chothia, or IMGT definition. In some embodiments, the anti-CCR2 antibody molecule comprises one, two, three, or four heavy chain framework regions (e.g., heavy chain FR1, FR2, FR3, and/or FR4) disclosed in Table 17, e.g., in one row of Table 17, or an amino acid sequence comprising no more than 1, 2, 3, 4, 5, or 6 modifications. In some embodiments, the anti-CCR2 antibody molecule comprises one, two, three, or four heavy chain framework regions (e.g., heavy chain FR1, FR2, FR3, and/or FR4) from a VH disclosed in Table 19. In some embodiments, the anti-CCR2 antibody molecule comprises a VH disclosed in Table 19, or an amino acid sequence having at least 90, 91, 92, 93, 94, 95, 96, 97, 98, or 99% identity thereto.

In some embodiments, the anti-CCR2 antibody molecule comprises one, two, or three light chain CDRs (e.g., LCDR1, LCDR2, and/or LCDR3) disclosed in Table 18, e.g., in one row of Table 18, or an amino acid sequence comprising no more than 1, 2, 3, 4, 5, or 6 modifications. In some embodiments, the anti-CCR2 antibody molecule comprises one, two, or three light chain CDRs (e.g., LCDR1, LCDR2, and/or LCDR3) from a light chain variable region (VL) disclosed in Table 20, e.g., optionally wherein the CDRs are according to the Kabat, Chothia, or IMGT definition. In some embodiments, the anti-CCR2 antibody molecule comprises one, two, three, or four light chain framework regions (e.g., light chain FR1, FR2, FR3, and/or FR4) disclosed in Table 18, e.g., in one row of Table 18, or an amino acid sequence comprising no more than 1, 2, 3, 4, 5, or 6 modifications. In some embodiments, the anti-CCR2 antibody molecule comprises one, two, three, or four light chain framework regions (e.g., light chain FR1, FR2, FR3, and/or FR4) from a VL disclosed in Table 20. In some embodiments, the anti-CCR2 antibody molecule comprises a VL disclosed in Table 20, or an amino acid sequence having at least 90, 91, 92, 93, 94, 95, 96, 97, 98, or 99% identity thereto. In some embodiments, the anti-CCR2 antibody molecule does not comprise a VH comprising the amino acid sequence of: EVQLVESGGGLVKPGGSLRLSCAASGFSFNAYAMNWVRQAPGKGLEWVGRIRTKNNN YATYYADSVKDRFTISRDDSKNTLYLQMNSLKTEDTAVYYCTTFYGNGVWGQGTLVT VSS (SEQ ID NO: 480). In some embodiments, the anti-CCR2 antibody molecule does not comprise a VL comprising the amino acid sequence of:

(SEQ ID NO: 481)
DVVMTQSPLSLPVTLGQPASISCKSSQSLLDSDGKTFLNWLLQRPGQSPR
RLIYLVSKLDSGVPDRFSGSGSGTDFTLKISRVEAEDVGVYYCWQGTHFP
YTFGGGTRLEIK.

In some embodiments, the anti-CCR2 antibody molecule comprises a VH encoded by a nucleotide sequence disclosed in Table 19 (or a nucleotide sequence having at least 80, 85, 90, or 95% identity thereto), and/or a VL encoded by a nucleotide sequence disclosed in Table 20 (or a nucleotide sequence having at least 80, 85, 90, or 95% identity thereto).

TABLE 17
CDRs and framework regions (FR) of exemplary heavy chain
variable regions of anti-CCR2 antibody molecules.

| VH | FR 1 | HCDR 1 | FR 2 | HCDR 2 | FR 3 | HCDR 3 | FR 4 |
|---|---|---|---|---|---|---|---|
| aCCR2 | EVQLVESG | GFSFNA | WVRQAPG | RIRTKNN | RFTISRDDSKS | FYGNGV | WGQGTT |
| Hum1 | GGLVKPGG | YAMN | KGLEWVG | NYATYYA | TLYLQMNSLKT | (SEQ | VTVSS |
| VH | SLRLSCAA | (SEQ | (SEQ | DSVKD | EDTAVYYCTT | ID NO: | (SEQ |
| (SEQ ID | S (SEQ | ID NO: | ID NO: | (SEQ ID | (SEQ ID NO: | 448) | ID NO: |
| NO: 343) | ID NO: | 446) | 450) | NO: 447) | 451) | | 453) |
| | 449) | | | | | | |
| aCCR2 | EVQLVESG | GFSFNA | WVRQAPG | RIRTKNN | RFTISRDDSKS | FYGNGV | WGQGTT |
| Hum2 | GGLVQPGG | YAMN | KGLEWVG | NYATYYA | SLYLQMNSLKT | (SEQ | VTVSS |
| VH | SLRLSCAA | (SEQ | (SEQ | DSVKD | EDTAVYYCAT | ID NO: | (SEQ |
| (SEQ ID | S (SEQ | ID NO: | ID NO: | (SEQ ID | (SEQ ID NO: | 448) | ID NO: |
| NO: 344) | ID NO: | 446) | 450) | NO: 447) | 452) | | 453) |
| | 424) | | | | | | |

TABLE 18
CDRs and framework regions (FR) of exemplary
light chain variable regions of anti-CCR2
antibody molecules.

| VL | FR 1 | LCDR 1 | FR 2 | LCDR 2 | FR 3 | LCDR 3 | FR 4 |
|---|---|---|---|---|---|---|---|
| aCCR2 | DVVMTQPS | KSSQSLL | WLQQRPG | LVSKLD | GVPDRFSGSG | WQGTH | FGGGTK |
| VL | LSLPVTLG | DSDGKTF | QSPRRLI | S (SEQ | SGTDFTLKIS | FPYT | VEIK |
| (SEQ | QPASISC | LN (SEQ | Y (SEQ | ID NO: | RVEAEDVGVY | (SEQ | (SEQ |
| ID NO: | (SEQ ID | ID NO: | ID NO: | 455) | YC (SEQ ID | ID NO: | ID NO: |
| 345) | NO: 457) | 454) | 458) | | NO: 459) | 456) | 460) |

TABLE 19
Amino acid and nucleotide sequences of exemplary heavy chain
variable regions of anti-CCR2 antibody molecules.

| SEQ ID NO | Description | Sequence |
|---|---|---|
| SEQ ID NO: | aCCR2 Hum1 | EVQLVESGGGLVKPGGSLRLSCAASGFSFNAYAMNWVRQAP |
| 343 | VH aa | GKGLEWVGRIRTKNNNYATYYADSVKDRFTISRDDSKSTLY |
| | | LQMNSLKTEDTAVYYCTTFYGNGVWGQGTTVTVSS |
| SEQ ID NO: | aCCR2 Hum2 | EVQLVESGGGLVQPGGSLRLSCAASGFSFNAYAMNWVRQAP |
| 344 | VH aa | GKGLEWVGRIRTKNNNYATYYADSVKDRFTISRDDSKSSLY |
| | | LQMNSLKTEDTAVYYCATFYGNGVWGQGTTVTVSS |
| SEQ ID NO: | aCCR2 Hum1 | GAAGTGCAGCTGGTGGAAAGCGGTGGCGGTCTGGTGCAGCC |
| 270 | VH nt | TGGCGGTTCCCTCCGGCTGTCATGTGCTGCCTCCGGATTCT |
| | | CGTTCAACGCGTATGCCATGAACTGGGTGAGGCAGGCCCCG |
| | | GGAAAGGGACTGGAATGGGTGGGGAGAATCCGGACCAAGAA |
| | | CAATAACTACGCTACTTACTACGCCGACTCCGTCAAGGACC |
| | | GGTTCACAATTTCCCGGGACGACAGCAAGAGCTCACTGTAC |
| | | CTTCAGATGAACAGCCTCAAAACCGAGGACACCGCCGTGTA |
| | | CTACTGTGCCACCTTTTACGGCAACGGTGTGTGGGGACAGG |
| | | GAACAACTGTCACCGTGTCCTCC |
| SEQ ID NO: | aCCR2 Hum2 | GAGGTGCAGCTGGTTGAATCTGGCGGAGGATTGGTTCAGCC |
| 271 | VH nt | TGGCGGCTCTCTGAGACTGTCTTGTGCCGCCTCTGGCTTCT |
| | | CCTTCAACGCCTACGCCATGAACTGGGTCCGACAGGCTCCT |
| | | GGCAAAGGCCTGGAATGGGTCGGAAGAATCCGGACCAAGAA |
| | | CAACAACTACGCCACCTACTACGCCGACTCCGTGAAGGACC |
| | | GGTTCACCATCTCTCGGGACGACTCCAAGTCCAGCCTGTAC |
| | | CTGCAGATGAACTCCCTGAAAACCGAGGACACCGCCGTGTA |
| | | CTACTGCGCCACCTTTTACGGCAATGGCGTGTGGGGCCAGG |
| | | GCACCACAGTTACAGTTTCTTCC |

TABLE 20
Amino acid and nucleotide sequences of exemplary light chain
variable regions of anti-CCR2 antibody molecules.

| SEQ ID NO | Description | Sequence |
|---|---|---|
| SEQ ID NO: | aCCR2 VL aa | DVVMTQSPLSLPVTLGQPASISCKSSQSLLDSDGKTFLNWLQ |
| 345 | | QRPGQSPRRLIYLVSKLDSGVPDRFSGSGSGTDFTLKISRVE |
| | | AEDVGVYYCWQGTHFPYTFGGGTKVEIK |
| SEQ ID NO: | aCCR2 VL nt | GACGTCGTCATGACCCAATCCCCGCTCTCCCTGCCGGTGACT |
| 272 | | CTCGGCCAGCCGGCGTCCATCTCGTGCAAGTCCTCCCAATCC |
| | | CTGCTGGATTCGGATGGGAAGACTTTCCTGAACTGGCTGCAG |
| | | CAACGGCCCGGCCAGTCGCCACGGCGGCTGATCTACCTGGTG |
| | | TCCAAGCTGGATTCAGGTGTGCCTGATAGATTCTCCGGCAGC |
| | | GGATCGGGGACCGACTTCACGCTGAAGATCTCCAGAGTGGAA |
| | | GCAGAGGACGTGGGCGTCTATTACTGTTGGCAGGGGACTCAT |
| | | TTCCCCTATACCTTTGGAGGTGGTACTAAGGTGGAAATTAAG |

### Antibody Molecules Targeting PD-L1

In some embodiments, provided herein are antibody molecules (e.g., monospecific or multispecific antibody molecules) that bind to PD-L1, e.g., human PD-L1.

In some embodiments, the anti-PD-L1 antibody molecule comprises one, two, or three heavy chain CDRs (e.g., HCDR1, HCDR2, and/or HCDR3) disclosed in Table 21, e.g., in one row of Table 21, or an amino acid sequence comprising no more than 1, 2, 3, 4, 5, or 6 modifications. In some embodiments, the anti-PD-L1 antibody molecule comprises one, two, or three heavy chain CDRs (e.g., HCDR1, HCDR2, and/or HCDR3) from a heavy chain variable region (VH) disclosed in Table 23, e.g., optionally wherein the CDRs are according to the Kabat, Chothia, or IMGT definition. In some embodiments, the anti-PD-L1 antibody molecule comprises one, two, three, or four heavy chain framework regions (e.g., heavy chain FR1, FR2, FR3, and/or FR4) disclosed in Table 21, e.g., in one row of Table 21, or an amino acid sequence comprising no more than 1, 2, 3, 4, 5, or 6 modifications. In some embodiments, the anti-PD-L1 antibody molecule comprises one, two, three, or four heavy chain framework regions (e.g., heavy chain FR1, FR2, FR3, and/or FR4) from a VH disclosed in Table 23. In some embodiments, the anti-PD-L1 antibody molecule comprises a VH disclosed in Table 23, or an amino acid sequence having at least 90, 91, 92, 93, 94, 95, 96, 97, 98, or 99% identity thereto.

In some embodiments, the anti-PD-L1 antibody molecule comprises one, two, or three light chain CDRs (e.g., LCDR1, LCDR2, and/or LCDR3) disclosed in Table 22, e.g., in one row of Table 22, or an amino acid sequence comprising no more than 1, 2, 3, 4, 5, or 6 modifications. In some embodiments, the anti-PD-L1 antibody molecule comprises one, two, or three light chain CDRs (e.g., LCDR1, LCDR2, and/or LCDR3) from a light chain variable region (VL) disclosed in Table 24, e.g., optionally wherein the CDRs are according to the Kabat, Chothia, or IMGT definition. In some embodiments, the anti-PD-L1 antibody molecule comprises one, two, three, or four light chain framework regions (e.g., light chain FR1, FR2, FR3, and/or FR4) disclosed in Table 22, e.g., in one row of Table 22, or an amino acid sequence comprising no more than 1, 2, 3, 4, 5, or 6 modifications. In some embodiments, the anti-PD-L1 antibody molecule comprises one, two, three, or four light chain framework regions (e.g., light chain FR1, FR2, FR3, and/or FR4) from a VL disclosed in Table 24. In some embodiments, the anti-PD-L1 antibody molecule comprises a VL disclosed in Table 24, or an amino acid sequence having at least 90, 91, 92, 93, 94, 95, 96, 97, 98, or 99% identity thereto.

In some embodiments, the anti-PD-L1 antibody molecule comprises a VH encoded by a nucleotide sequence disclosed in Table 23 (or a nucleotide sequence having at least 80, 85, 90, or 95% identity thereto), and/or a VL encoded by a nucleotide sequence disclosed in Table 24 (or a nucleotide sequence having at least 80, 85, 90, or 95% identity thereto).

TABLE 21
CDRs and framework regions (FR) of exemplary heavy chain
variable regions of anti-PD-L1 antibody molecules.

| VH | FR 1 | HCDR 1 | FR 2 | HCDR 2 | FR 3 | HCDR 3 | FR 4 |
|---|---|---|---|---|---|---|---|
| aPDL1 | QVQLVQSG | GGTFSS | WVRQAPG | GIIPIFGT | RVTITADKST | GGYSYG | WGQGTL |
| BH794 | AEVKKPGS | YAIS | QGLEWMG | ANYAQKFQ | STAYMELSSL | SLDY | VTVSS |
| VH | SVKVSCKA | (SEQ | (SEQ | G (SEQ | RSEDTAVYYC | (SEQ | (SEQ |
| (SEQ | S (SEQ | ID NO: | ID NO: | ID NO: | AR (SEQ ID | ID NO: | ID NO: |
| ID NO: | ID NO: | 461) | 465) | 462) | NO: 466) | 463) | 430) |
| 346) | 464) | | | | | | |

TABLE 22
CDRs and framework regions (FR) of exemplary
light chain variable regions of anti-PD-L1
antibody molecules.

| VL | FR 1 | LCDR 1 | FR 2 | LCDR 2 | FR 3 | LCDR 3 | FR 4 |
|---|---|---|---|---|---|---|---|
| aPDL1 | DIQMTQS | RASQSI | WYQQKPG | AASSLQ | GVPSRFSGS | QQSYST | FGQGTK |
| BH794 | PSSLSAS | ISDLN | KAPKLLI | S (SEQ | GSGTDFTLT | PYT | LEIK |
| VL | VGDRVTI | (SEQ | H (SEQ | ID NO: | ISSLQPEDF | (SEQ | (SEQ |
| (SEQ | TC SEQ | ID NO: | ID NO: | 468) | ATYYC | ID NO: | ID NO: |
| ID NO: | ID NO: | 467) | 471) | | (SEQ ID | 469) | 473) |
| 347) | 470 | | | | NO: 472) | | |

TABLE 23
Amino acid and nucleotide sequences of exemplary heavy chain
variable regions of anti-PD-L1 antibody molecules.

| SEQ ID NO | Description | Sequence |
|---|---|---|
| SEQ ID NO: | aPDL1 | QVQLVQSGAEVKKPGSSVKVSCKASGGTFSSYAISWVRQAPGQGLE |
| 346 | BH794 VH | WMGGIIPIFGTANYAQKFQGRVTITADKSTSTAYMELSSLRSEDTA |
| | aa | VYYCARGGYSYGSLDYWGQGTLVTVSS |
| SEQ ID NO: | aPDL1 | CAGGTGCAGCTGGTTCAGTCTGGCGCCGAAGTGAAGAAACCTGGCT |
| 273 | BH794 VH | CCTCCGTGAAGGTGTCCTGCAAGGCTTCTGGCGGCACCTTCTCCTC |
| | nt | TTACGCCATCTCCTGGGTCCGACAGGCTCCTGGACAAGGCTTGGAA |
| | | TGGATGGGCGGCATCATCCCCATCTTCGGCACCGCCAATTACGCCC |
| | | AGAAATTCCAGGGCAGAGTGACCATCACCGCCGACAAGTCTACCTC |
| | | CACCGCCTACATGGAACTGTCCAGCCTGAGATCTGAGGACACCGCC |
| | | GTGTACTACTGTGCCAGAGGCGGCTACTCTTACGGCTCCCTGGATT |
| | | ATTGGGGCCAGGGCACACTGGTCACCGTGTCCTCT |

TABLE 24
Amino acid and nucleotide sequences of exemplary light chain
variable regions of anti-PD-L1 antibody molecules.

| SEQ ID NO | Description | Sequence |
|---|---|---|
| SEQ ID NO: | aPDL1 | DIQMTQSPSSLSASVGDRVTITCRASQSIISDLNWYQQKPGKAPKL |
| 347 | BH794 VL aa | LIHAASSLQSGVPSRFSGSGSGTDFTLTISSLQPEDFATYYCQQSY |
| | | STPYTFGQGTKLEIK |
| SEQ ID NO: | aPDL1 | GACATCCAGATGACCCAGTCTCCATCCTCTCTGTCCGCCTCTGTGG |
| 274 | BH794 VL nt | GCGACAGAGTGACCATCACCTGTCGGGCCTCTCAGTCCATCATCTC |
| | | CGACCTGAACTGGTATCAGCAGAAGCCCGGCAAGGCCCCTAAGCTG |
| | | CTGATCCATGCTGCTTCCAGTCTGCAGTCTGGCGTGCCCTCTAGAT |
| | | TCTCCGGCTCTGGCTCTGGCACCGACTTTACCCTGACAATCTCCAG |
| | | CCTGCAGCCTGAGGACTTCGCCACCTACTACTGCCAGCAGTCCTAC |
| | | AGCACCCCTTACACCTTTGGCCAGGGCACCAAGCTGGAAATCAAG |

### Multispecific Antibody Molecules

In embodiments, multispecific antibody molecules can comprise more than one antigen-binding site, where different sites are specific for different antigens. In embodiments, multispecific antibody molecules can bind more than one (e.g., two or more) epitopes on the same antigen. In embodiments, multispecific antibody molecules comprise an antigen-binding site specific for a target cell (e.g., cancer cell) and a different antigen-binding site specific for an immune effector cell. In one embodiment, the multispecific antibody molecule is a bispecific antibody molecule. Bispecific antibody molecules can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates.

BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, κλ-body, orthogonal Fab. See Spiess et al. Mol. Immunol. 67(2015):95-106. Exemplary BsIgGs include catumaxomab (Fresenius Biotech, Trion Pharma, Neopharm), which contains an anti-CD3 arm and an anti-EpCAM arm; and ertumaxomab (Neovii Biotech, Fresenius Biotech), which targets CD3 and HER2. In some embodiments, BsIgG comprises heavy chains that are engineered for heterodimerization. For example, heavy chains can be engineered for heterodimerization using a “knobs-into-holes” strategy, a SEED platform, a common heavy chain (e.g., in Kk-bodies), and use of heterodimeric Fc regions. See Spiess et al. Mol. Immunol. 67(2015):95-106. Strategies that have been used to avoid heavy chain pairing of homodimers in BsIgG include knobs-in-holes, duobody, azymetric, charge pair, HA-TF, SEEDbody, and differential protein A affinity. See Id. BsIgG can be produced by separate expression of the component antibodies in different host cells and subsequent purification/assembly into a BsIgG. BsIgG can also be produced by expression of the component antibodies in a single host cell. BsIgG can be purified using affinity chromatography, e.g., using protein A and sequential pH elution.

IgG appended with an additional antigen-binding moiety is another format of bispecific antibody molecules. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable regions (e.g., single chain variable fragments or variable fragments). See Id. Examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. An example of an IgG-scFv is MM-141 (Merrimack Pharmaceuticals), which binds IGF-1R and HER3. Examples of DVD-Ig include ABT-981 (AbbVie), which binds IL-1α and IL-1β; and ABT-122 (AbbVie), which binds TNF and IL-17A.

Bispecific antibody fragments (BsAb) are a format of bispecific antibody molecules that lack some or all of the antibody constant domains. For example, some BsAb lack an Fc region. In embodiments, bispecific antibody fragments include heavy and light chain regions that are connected by a peptide linker that permits efficient expression of the BsAb in a single host cell. Exemplary bispecific antibody fragments include but are not limited to nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, and intrabody. See Id For example, the BiTE format comprises tandem scFvs, where the component scFvs bind to CD3 on T cells and a surface antigen on cancer cells

Bispecific fusion proteins include antibody fragments linked to other proteins, e.g., to add additional specificity and/or functionality. An example of a bispecific fusion protein is an immTAC, which comprises an anti-CD3 scFv linked to an affinity-matured T-cell receptor that recognizes HLA-presented peptides. In embodiments, the dock-and-lock (DNL) method can be used to generate bispecific antibody molecules with higher valency. Also, fusions to albumin binding proteins or human serum albumin can be extend the serum half-life of antibody fragments. See Id

In embodiments, chemical conjugation, e.g., chemical conjugation of antibodies and/or antibody fragments, can be used to create BsAb molecules. See Id An exemplary bispecific antibody conjugate includes the CovX-body format, in which a low molecular weight drug is conjugated site-specifically to a single reactive lysine in each Fab arm or an antibody or fragment thereof. In embodiments, the conjugation improves the serum half-life of the low molecular weight drug. An exemplary CovX-body is CVX-241 (NCT01004822), which comprises an antibody conjugated to two short peptides inhibiting either VEGF or Ang2. See Id

The antibody molecules can be produced by recombinant expression, e.g., of at least one or more component, in a host system. Exemplary host systems include eukaryotic cells (e.g., mammalian cells, e.g., CHO cells, or insect cells, e.g., SF9 or S2 cells) and prokaryotic cells (e.g., E. coli). Bispecific antibody molecules can be produced by separate expression of the components in different host cells and subsequent purification/assembly. Alternatively, the antibody molecules can be produced by expression of the components in a single host cell. Purification of bispecific antibody molecules can be performed by various methods such as affinity chromatography, e.g., using protein A and sequential pH elution. In other embodiments, affinity tags can be used for purification, e.g., histidine-containing tag, myc tag, or streptavidin tag.

### Various Embodiments of Multispecific Antibody Molecules

In one aspect, provided herein is a multispecific antibody molecule comprising an anti-CSF1R binding moiety. In one embodiment, the anti-CSF1R binding moiety comprises an anti-CSF1R antibody molecule disclosed herein, e.g., an anti-CSF1R antibody molecule disclosed in the section with the subtitle “Antibody molecules targeting CSF1R.” In some embodiments, the multispecific antibody molecule further comprises an anti-CCR2 binding moiety (e.g., an anti-CCR2 antibody molecule disclosed herein), an anti-PD-L1 binding moiety (e.g., an anti-PD-L1 antibody molecule disclosed herein), a TGF beta inhibitor, and/or a cytokine molecule (e.g., an IL-2 molecule).

In one aspect, provided herein is a multispecific antibody molecule comprising an anti-CCR2 binding moiety. In one embodiment, the anti-CCR2 binding moiety comprises an anti-CCR2 antibody molecule disclosed herein, e.g., an anti-CCR2 antibody molecule disclosed in the section with the subtitle “Antibody molecules targeting CCR2.” In some embodiments, the multispecific antibody molecule further comprises an anti-CSF1R binding moiety (e.g., an anti-CSF1R antibody molecule disclosed herein), an anti-PD-L1 binding moiety (e.g., an anti-PD-L1 antibody molecule disclosed herein), a TGF beta inhibitor, and/or a cytokine molecule (e.g., an IL-2 molecule).

In one aspect, provided herein is a multispecific antibody molecule comprising an anti-PD-L1 binding moiety. In one embodiment, the anti-PD-L1 binding moiety comprises an anti-PD-L1 antibody molecule disclosed herein, e.g., an anti-PD-L1 antibody molecule disclosed in the section with the subtitle “Antibody molecules targeting PD-L1.” In some embodiments, the multispecific antibody molecule further comprises an anti-CSF1R binding moiety (e.g., an anti-CSF1R antibody molecule disclosed herein), an anti-CCR2 binding moiety (e.g., an anti-CCR2 antibody molecule disclosed herein), a TGF beta inhibitor, and/or a cytokine molecule (e.g., an IL-2 molecule).

In some embodiments, the multispecific antibody molecule comprises an anti-CSF1R binding moiety and an anti-CCR2 binding moiety. In some embodiments, the multispecific antibody molecule comprises an amino acid sequence disclosed in Table 25, or a fragment thereof. In some embodiments, the multispecific antibody molecule is or comprises a multispecific antibody molecule disclosed herein, e.g., BIM0648 or BIM0652 disclosed in Table 34. In some embodiments, the multispecific antibody molecule comprises one or more CDRs, one or more heavy chain variable regions, one or more light chain variable regions, one or more heavy chains, and/or one or more light chains of BIM0648 or BIM0652 disclosed in Table 34. In some embodiments, the multispecific antibody molecule is or comprises a multispecific antibody molecule disclosed herein, e.g., any of molecules 1-86 disclosed herein, e.g., any of molecules 10-86 disclosed in Table 28, e.g., any of molecules BIM0204, BIM0205, BIM0206, BIM0207, BIM0208, BIM0209, BIM0210, BIM0211, BIM0542, BIM0543, BIM0544, BIM0545, BIM0546, BIM0547, BIM0548, BIM0549, BIM0550, BIM0551, BIM0552, BIM0553, BIM0554, BIM0555, BIM0556, BIM0566, and BIM0567 disclosed in Table 28. In some embodiments, the multispecific antibody molecule comprises one or more CDRs, one or more heavy chain variable regions, one or more light chain variable regions, one or more heavy chains, and/or one or more light chains of any of molecules BIM0204, BIM0205, BIM0206, BIM0207, BIM0208, BIM0209, BIM0210, BIM0211, BIM0542, BIM0543, BIM0544, BIM0545, BIM0546, BIM0547, BIM0548, BIM0549, BIM0550, BIM0551, BIM0552, BIM0553, BIM0554, BIM0555, BIM0556, BIM0566, and BIM0567 disclosed in Table 28.

TABLE 25
Amino acid and nucleotide sequences of exemplary
multispecific antibody molecules, e.g., targeting
CSF1R, CCR2, or PD-L1.

| SEQ ID | | |
|---|---|---|
| NO | Description | Sequence |
| SEQ ID | aCSF1R | EVQLVESGGGLVQPGGSLRLSCAASGFTFSNDEMNWIRQAPGKGLEWVSYI |
| NO: | BI117 VH- | SSRSSTIYYADAVKGRFTISRDNARNSLYLQMNSLRAEDTAVYYCARGVDG |
| 351 | hCH1- | DFSFDYWGQGTLVTVSSASTKGPSVFPLAPSSKSTSGGTAALGCLVKDYFP |
| | hFc_Hole | EPVTVSWNSGALTSGVHTFPAVLQSSGLYSLSSVVTVPSSSLGTQTYICNV |
| | | NHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVCTLPPSR |
| | | EEMTKNQVSLSCAVKGFYPSDIAVEWESNGQPENNYKTTPPVLDSDGSFFL |
| | | VSKLTVDKSRWQQGNVFSCSVMHEALHNHYTQKSLSLSPGX, wherein |
| | | X is K or absent |
| SEQ ID | aCSF1R | EVQLVESGGGLVQPGGSLRLSCAASGFTFSNDEMNWIRQAPGKGLEWVSYI |
| NO: | BI117 C5- | SSGSSTIYYADAVKGRFTISRDNARNSLYLQMNSLRAEDTAVYYCARGVDG |
| 352 | R2A VH- | DFSFDYWGQGTLVTVSSASTKGPSVFPLAPSSKSTSGGTAALGCLVKDYFP |
| | hCH1- | EPVTVSWNSGALTSGVHTFPAVLQSSGLYSLSSVVTVPSSSLGTQTYICNV |
| | hFc_Hole | NHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVCTLPPSR |
| | | EEMTKNQVSLSCAVKGFYPSDIAVEWESNGQPENNYKTTPPVLDSDGSFFL |
| | | VSKLTVDKSRWQQGNVFSCSVMHEALHNHYTQKSLSLSPGX, wherein |
| | | X is K or absent |
| SEQ ID | aCSF1R | EVQLVESGGGLVQPGGSLRLSCAASGFTFSNDEMNWIRQAPGKGLEWVSFI |
| NO: | BI117 C9- | SGGSSTIYYADAVKGRFTISRDNARNSLYLQMNSLRAEDTAVYYCARGVDG |
| 353 | R2A VH- | DFSFDYWGQGTLVTVSSASTKGPSVFPLAPSSKSTSGGTAALGCLVKDYFP |
| | hCH1- | EPVTVSWNSGALTSGVHTFPAVLQSSGLYSLSSVVTVPSSSLGTQTYICNV |
| | hFc_Hole | NHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVCTLPPSR |
| | | EEMTKNQVSLSCAVKGFYPSDIAVEWESNGQPENNYKTTPPVLDSDGSFFL |
| | | VSKLTVDKSRWQQGNVFSCSVMHEALHNHYTQKSLSLSPGX, wherein |
| | | X is K or absent |
| SEQ ID | aCSF1R | EVQLVESGGGLVQPGGSLRLSCAASGFTFSNDEMNWIRQAPGKGLEWVSYI |
| NO: | BI117 D1- | SSRSSTIYYADAVKGRFTISRDNARNSLYLQMNSLRAEDTAVYYCARNSDE |
| 354 | R2A VH- | DGSFDYWGQGTLVTVSSASTKGPSVFPLAPSSKSTSGGTAALGCLVKDYFP |
| | hCH1- | EPVTVSWNSGALTSGVHTFPAVLQSSGLYSLSSVVTVPSSSLGTQTYICNV |
| | hFc_Hole | NHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVCTLPPSR |
| | | EEMTKNQVSLSCAVKGFYPSDIAVEWESNGQPENNYKTTPPVLDSDGSFFL |
| | | VSKLTVDKSRWQQGNVFSCSVMHEALHNHYTQKSLSLSPGX, wherein |
| | | X is K or absent |
| SEQ ID | aCSF1R | EVQLVESGGGLVQPGGSLRLSCAASGFTFSNDEMNWIRQAPGKGLEWVSYI |
| NO: | BI117 E2- | TSKSSTIYYADSVKGRFTISRDNARNSLYLQMNSLRAEDTAVYYCARGVDG |
| 355 | R2B VH- | DFSFDYWGQGTLVTVSSASTKGPSVFPLAPSSKSTSGGTAALGCLVKDYFP |
| | hCH1- | EPVTVSWNSGALTSGVHTFPAVLQSSGLYSLSSVVTVPSSSLGTQTYICNV |
| | hFc_Hole | NHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVCTLPPSR |
| | | EEMTKNQVSLSCAVKGFYPSDIAVEWESNGQPENNYKTTPPVLDSDGSFFL |
| | | VSKLTVDKSRWQQGNVFSCSVMHEALHNHYTQKSLSLSPGX, wherein |
| | | X is K or absent |
| SEQ ID | aCSF1R | EVQLVESGGGLVQPGGSLRLSCAASGFTFSNDEMNWIRQAPGKGLEWVSYI |
| NO: | BI117 G1- | SSRSSTIYYADAVKGRFTISRDNARNSLYLQMNSLRAEDTAVYYCARGVDG |
| 356 | R2B VH- | DFSFDVWGQGTLVTVSSASTKGPSVFPLAPSSKSTSGGTAALGCLVKDYFP |
| | hCH1- | EPVTVSWNSGALTSGVHTFPAVLQSSGLYSLSSVVTVPSSSLGTQTYICNV |
| | hFc_Hole | NHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVCTLPPSR |
| | | EEMTKNQVSLSCAVKGFYPSDIAVEWESNGQPENNYKTTPPVLDSDGSFFL |
| | | VSKLTVDKSRWQQGNVFSCSVMHEALHNHYTQKSLSLSPGX, wherein |
| | | X is K or absent |
| SEQ ID | aCSF1R | EVQLVESGGGLVQPGGSLRLSCAASGFTFSNDEMNWIRQAPGKGLEWVSYI |
| NO: | BI117 | TSKSSTIYYADAVKGRFTISRDNARNSLYLQMNSLRAEDTAVYYCARGVDG |
| 357 | G12-R2B | DFSFDYWGQGTLVTVSSASTKGPSVFPLAPSSKSTSGGTAALGCLVKDYFP |
| | VH-hCH1- | EPVTVSWNSGALTSGVHTFPAVLQSSGLYSLSSVVTVPSSSLGTQTYICNV |
| | hFc_Hole | NHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVCTLPPSR |
| | | EEMTKNQVSLSCAVKGFYPSDIAVEWESNGQPENNYKTTPPVLDSDGSFFL |
| | | VSKLTVDKSRWQQGNVFSCSVMHEALHNHYTQKSLSLSPGX, wherein |
| | | X is K or absent |
| SEQ ID | aCSF1R | EVQLVESGGGLVQPGGSLRLSCAASGFTFSNDEMNWIRQAPGKGLEWVSYI |
| NO: | BI117 D6- | SSRSSTIYYADAVKGRFTISRDNARNSLYLQMNSLRAEDTAVYYCARSVDG |
| 358 | R3B VH- | DGSFDYWGQGTLVTVSSASTKGPSVFPLAPSSKSTSGGTAALGCLVKDYFP |
| | hCH1- | EPVTVSWNSGALTSGVHTFPAVLQSSGLYSLSSVVTVPSSSLGTQTYICNV |
| | hFc_Hole | NHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVCTLPPSR |
| | | EEMTKNQVSLSCAVKGFYPSDIAVEWESNGQPENNYKTTPPVLDSDGSFFL |
| | | VSKLTVDKSRWQQGNVFSCSVMHEALHNHYTQKSLSLSPGX, wherein |
| | | X is K or absent |
| SEQ ID | aCSF1R | EVQLVESGGGLVQPGGSLRLSCAASGFTFSNDEMNWIRQAPGKGLEWVSYI |
| NO: | BI117 A6- | SSRSSTIYYADAVKGRFTISRDNARNSLYLQMNSLRAEDTAVYYCARSSDG |
| 359 | R3B VH- | STSFDYWGQGTLVTVSSASTKGPSVFPLAPSSKSTSGGTAALGCLVKDYFP |
| | hCH1- | EPVTVSWNSGALTSGVHTFPAVLQSSGLYSLSSVVTVPSSSLGTQTYICNV |
| | hFc_Hole | NHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVCTLPPSR |
| | | EEMTKNQVSLSCAVKGFYPSDIAVEWESNGQPENNYKTTPPVLDSDGSFFL |
| | | VSKLTVDKSRWQQGNVFSCSVMHEALHNHYTQKSLSLSPGX, wherein |
| | | X is K or absent |
| SEQ ID | aCSF1R | EVQLVESGGGLVQPGGSLRLSCAASGFTFSNDEMNWIRQAPGKGLEWVSYI |
| NO: | BI117 | SSSSSTIYYADAVTGRFTISRDNARNSLYLQMNSLRAEDTAVYYCARGVDG |
| 360 | A11-R3B | DFSFDYWGQGTLVTVSSASTKGPSVFPLAPSSKSTSGGTAALGCLVKDYFP |
| | VH-hCH1- | EPVTVSWNSGALTSGVHTFPAVLQSSGLYSLSSVVTVPSSSLGTQTYICNV |
| | hFc_Hole | NHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVCTLPPSR |
| | | EEMTKNQVSLSCAVKGFYPSDIAVEWESNGQPENNYKTTPPVLDSDGSFFL |
| | | VSKLTVDKSRWQQGNVFSCSVMHEALHNHYTQKSLSLSPGX, wherein |
| | | X is K or absent |
| SEQ ID | aCSF1R | EVQLVESGGGLVQPGGSLRLSCAASGFTFSNDEMNWIRQAPGKGLEWVSYI |
| NO: | BI117 B2- | SSRSSTIYYADAVKGRFTISRDNARNSLYLQMNSLRAEDTAVYYCARNSDS |
| 361 | R3B VH- | DGSFDIWGQGTLVTVSSASTKGPSVFPLAPSSKSTSGGTAALGCLVKDYFP |
| | hCH1- | EPVTVSWNSGALTSGVHTFPAVLQSSGLYSLSSVVTVPSSSLGTQTYICNV |
| | hFc_Hole | NHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVCTLPPSR |
| | | EEMTKNQVSLSCAVKGFYPSDIAVEWESNGQPENNYKTTPPVLDSDGSFFL |
| | | VSKLTVDKSRWQQGNVFSCSVMHEALHNHYTQKSLSLSPGX, wherein |
| | | X is K or absent |
| SEQ ID | aCSF1R | EVQLVESGGGLVQPGGSLRLSCAASGFTFSNDEMNWIRQAPGKGLEWVSYI |
| NO: | BI117 C5- | SSRSSTIYYADAVKGRFTISRDNARNSLYLQMNSLRAEDTAVYYCARGVDG |
| 362 | R3B VH- | DFSFDFWGQGTLVTVSSASTKGPSVFPLAPSSKSTSGGTAALGCLVKDYFP |
| | hCH1- | EPVTVSWNSGALTSGVHTFPAVLQSSGLYSLSSVVTVPSSSLGTQTYICNV |
| | hFc_Hole | NHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVCTLPPSR |
| | | EEMTKNQVSLSCAVKGFYPSDIAVEWESNGQPENNYKTTPPVLDSDGSFFL |
| | | VSKLTVDKSRWQQGNVFSCSVMHEALHNHYTQKSLSLSPGX, wherein |
| | | X is K or absent |
| SEQ ID | aCSF1R | EVQLVESGGGLVQPGGSLRLSCAASGFTFSNDEMNWIRQAPGKGLEWVSYI |
| NO: | BI117 C6- | SSRSSTIYYADAVKGRFTISRDNARNSLYLQMNSLRAEDTAVYYCARSGRL |
| 363 | R3B VH- | DGSFDYWGQGTLVTVSSASTKGPSVFPLAPSSKSTSGGTAALGCLVKDYFP |
| | hCH1- | EPVTVSWNSGALTSGVHTFPAVLQSSGLYSLSSVVTVPSSSLGTQTYICNV |
| | hFc_Hole | NHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVCTLPPSR |
| | | EEMTKNQVSLSCAVKGFYPSDIAVEWESNGQPENNYKTTPPVLDSDGSFFL |
| | | VSKLTVDKSRWQQGNVFSCSVMHEALHNHYTQKSLSLSPGX, wherein |
| | | X is K or absent |
| SEQ ID | aCSF1R | EVQLVESGGGLVQPGGSLRLSCAASGFTFSNDEMNWIRQAPGKGLEWVSYI |
| NO: | BI117 | SSRSSTIYYADAVKGRFTISRDNARNSLYLQMNSLRAEDTAVYYCARGVES |
| 364 | D11-R3B | DFSFDYWGQGTLVTVSSASTKGPSVFPLAPSSKSTSGGTAALGCLVKDYFP |
| | VH-hCH1- | EPVTVSWNSGALTSGVHTFPAVLQSSGLYSLSSVVTVPSSSLGTQTYICNV |
| | hFc_Hole | NHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVCTLPPSR |
| | | EEMTKNQVSLSCAVKGFYPSDIAVEWESNGQPENNYKTTPPVLDSDGSFFL |
| | | VSKLTVDKSRWQQGNVFSCSVMHEALHNHYTQKSLSLSPGX, wherein |
| | | X is K or absent |
| SEQ ID | aCSF1R | QVQLVQSGGGVVQPGRSLRLSCAASGFTFSSYAMHWVRQAPGKGLEWVAVI |
| NO: | BI123 VH- | SYDGSNKYYADSVKGRFTISRDNSKNTLYLQMNSLRAEDTAVYYCARELDY |
| 365 | hCH1- | GDYGLDVWGKGTTVTVSSASTKGPSVFPLAPSSKSTSGGTAALGCLVKDYF |
| | hFc_Hole | PEPVTVSWNSGALTSGVHTFPAVLQSSGLYSLSSVVTVPSSSLGTQTYICN |
| | | VNHKPSNTKVDKRVEPKSCDKTHTCPPCPAPELLGGPSVFLFPPKPKDTLM |
| | | ISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVCTLPPS |
| | | REEMTKNQVSLSCAVKGFYPSDIAVEWESNGQPENNYKTTPPVLDSDGSFF |
| | | LVSKLTVDKSRWQQGNVFSCSVMHEALHNHYTQKSLSLSPGX, wherein |
| | | X is K or absent |
| SEQ ID | aCSF1R | QVQLVQSGGGVVQPGRSLRLSCAASGFTFSSYAMHWVRQAPGKGLEWVAVI |
| NO: | BI123 A5- | SYDGSNKYYADSVKGRFTISRDNSKNTLYLQMNSLRAEDTAVYYCARELDY |
| 366 | R3B VH- | GDWGLDVWGKGTTVTVSSASTKGPSVFPLAPSSKSTSGGTAALGCLVKDYF |
| | hCH1- | PEPVTVSWNSGALTSGVHTFPAVLQSSGLYSLSSVVTVPSSSLGTQTYICN |
| | hFc_Hole | VNHKPSNTKVDKRVEPKSCDKTHTCPPCPAPELLGGPSVFLFPPKPKDTLM |
| | | ISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVCTLPPS |
| | | REEMTKNQVSLSCAVKGFYPSDIAVEWESNGQPENNYKTTPPVLDSDGSFF |
| | | LVSKLTVDKSRWQQGNVFSCSVMHEALHNHYTQKSLSLSPGX, wherein |
| | | X is K or absent |
| SEQ ID | aCSF1R | QVQLVQSGGGVVQPGRSLRLSCAASGFTFSSYAMHWVRQAPGKGLEWVAVI |
| NO: | BI123 B6- | PYYGSNKYYADSVKGRFTISRDNSKNTLYLQMNSLRAEDTAVYYCARELDY |
| 367 | R3B VH- | GDYGLDVWGKGTTVTVSSASTKGPSVFPLAPSSKSTSGGTAALGCLVKDYF |
| | hCH1- | PEPVTVSWNSGALTSGVHTFPAVLQSSGLYSLSSVVTVPSSSLGTQTYICN |
| | hFc_Hole | VNHKPSNTKVDKRVEPKSCDKTHTCPPCPAPELLGGPSVFLFPPKPKDTLM |
| | | ISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVCTLPPS |
| | | REEMTKNQVSLSCAVKGFYPSDIAVEWESNGQPENNYKTTPPVLDSDGSFF |
| | | LVSKLTVDKSRWQQGNVFSCSVMHEALHNHYTQKSLSLSPGX, wherein |
| | | X is K or absent |
| SEQ ID | aCSF1R | QVQLVQSGGGVVQPGRSLRLSCAASGFTFSSYAMHWVRQAPGKGLEWVAVI |
| NO: | BI123 C1- | SYDGSDKYYADSVKGRFTISRDNSKNTLYLQMNSLRAEDTAVYYCARELDY |
| 368 | R3B VH- | GDYGLDVWGKGTTVTVSSASTKGPSVFPLAPSSKSTSGGTAALGCLVKDYF |
| | hCH1- | PEPVTVSWNSGALTSGVHTFPAVLQSSGLYSLSSVVTVPSSSLGTQTYICN |
| | hFc_Hole | VNHKPSNTKVDKRVEPKSCDKTHTCPPCPAPELLGGPSVFLFPPKPKDTLM |
| | | ISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVCTLPPS |
| | | REEMTKNQVSLSCAVKGFYPSDIAVEWESNGQPENNYKTTPPVLDSDGSFF |
| | | LVSKLTVDKSRWQQGNVFSCSVMHEALHNHYTQKSLSLSPGX, wherein |
| | | X is K or absent |
| SEQ ID | aCSF1R | QLVLTQPPSVSGAPGQRVTISCTGSSSNIGAGYDVHWYQQLPGTAPKLLIY |
| NO: | BI117 VL- | GNSNRPSGVPDRFSGSKSGTSASLAITGLQAEDEADYYCQSYDSSLSEEVF |
| 369 | hCLIg_v1 | GGGTKLTVLGQPKANPTVTLFPPSSEELQANKATLVCLISDFYPGAVTVAW |
| | | KADGSPVKAGVETTKPSKQSNNKYAASSYLSLTPEQWKSHRSYSCQVTHEG |
| | | STVEKTVAPTECS |
| SEQ ID | aCSF1R | SLVLTQPPSVSVAPGKTARITCGGNNIGSKSVHWYQQKPGQAPVLVVYDDS |
| NO: | BI123 VL- | DRPSGIPERFSGSNSGNTATLTISRVEAGDEADYYCQVWDSSSDHVVFGGG |
| 370 | hCLIg_v1 | TQLTVLGQPKANPTVTLFPPSSEELQANKATLVCLISDFYPGAVTVAWKAD |
| | | GSPVKAGVETTKPSKQSNNKYAASSYLSLTPEQWKSHRSYSCQVTHEGSTV |
| | | EKTVAPTECS |
| SEQ ID | aCCR2 | EVQLVESGGGLVKPGGSLRLSCAASGFSFNAYAMNWVRQAPGKGLEWVGRI |
| NO: | Hum1 VH- | RTKNNNYATYYADSVKDRFTISRDDSKSTLYLQMNSLKTEDTAVYYCTTFY |
| 371 | hCH1- | GNGVWGQGTTVTVSSASTKGPSVFPLAPSSKSTSGGTAALGCLVKDYFPEP |
| | hFc_Knob | VTVSWNSGALTSGVHTFPAVLQSSGLYSLSSVVTVPSSSLGTQTYICNVNH |
| | | KPSNTKVDKRVEPKSCDKTHTCPPCPAPELLGGPSVFLFPPKPKDTLMISR |
| | | TPEVTCVVVDVSHEDPEVKFNWYVDGVEVHNAKTKPREEQYNSTYRVVSVL |
| | | TVLHQDWLNGKEYKCKVSNKALPAPIEKTISKAKGQPREPQVYTLPPCREE |
| | | MTKNQVSLWCLVKGFYPSDIAVEWESNGQPENNYKTTPPVLDSDGSFFLYS |
| | | KLTVDKSRWQQGNVFSCSVMHEALHNHYTQKSLSLSPGX, wherein X |
| | | is K or absent |
| SEQ ID | aCCR2 | EVQLVESGGGLVQPGGSLRLSCAASGFSFNAYAMNWVRQAPGKGLEWVGRI |
| NO: | Hum2 VH- | RTKNNNYATYYADSVKDRFTISRDDSKSSLYLQMNSLK1EDTAVYYCATFY |
| 372 | hCH1- | GNGVWGQGTTVTVSSASTKGPSVFPLAPSSKSTSGGTAALGCLVKDYFPEP |
| | hFc_Knob | VTVSWNSGALTSGVHTFPAVLQSSGLYSLSSVVTVPSSSLGTQTYICNVNH |
| | | KPSNTKVDKRVEPKSCDKTHTCPPCPAPELLGGPSVFLFPPKPKDTLMISR |
| | | TPEVTCVVVDVSHEDPEVKFNWYVDGVEVHNAKTKPREEQYNSTYRVVSVL |
| | | TVLHQDWLNGKEYKCKVSNKALPAPIEKTISKAKGQPREPQVYTLPPCREE |
| | | MTKNQVSLWCLVKGFYPSDIAVEWESNGQPENNYKTTPPVLDSDGSFFLYS |
| | | KLTVDKSRWQQGNVFSCSVMHEALHNHYTQKSLSLSPGX, wherein X |
| | | is K or absent |
| SEQ ID | aCCR2 | DVVMTQSPLSLPVTLGQPASISCKSSQSLLDSDGKTFLNWLQQRPGQSPRR |
| NO: | VL- | LIYLVSKLDSGVPDRFSGSGSGTDFTLKISRVEAEDVGVYYCWQGTHFPYT |
| 373 | hCLIg_vk | FGGGTKVEIKRTVAAPSVFIFPPSDEQLKSGTASVVCLLNNFYPREAKVQW |
| | | KVDNALQSGNSQESVIEQDSKDSTYSLSSTLTLSKADYEKHKVYACEVTHQ |
| | | GLSSPVTKSFNRGEC |
| SEQ ID | aCSF1R | EVQLVESGGGLVQPGGSLRLSCAASGFTFSNDEMNWIRQAPGKGLEWVSYI |
| NO: | BI117 VH- | SSRSSTIYYADAVKGRFTISRDNARNSLYLQMNSLRAEDTAVYYCARGVDG |
| 374 | hCH1- | DFSFDYWGQGTLVTVSSASTKGPSVFPLAPSSKSTSGGTAALGCLVKDYFP |
| | hFc_Hole- | EPVTVSWNSGALTSGVHTFPAVLQSSGLYSLSSVVTVPSSSLGTQTYICNV |
| | 3 x 4GS- | NHKPSNTKVDKRVEPKSCDKTHTCPPCPAPELLGGPSVFLFPPKPKDTLMI |
| | BH794_VH- | SRTPEVTCVVVDVSHEDPEVKFNWYVDGVEVHNAKTKPREEQYNSTYRVVS |
| | 3 x 4GS- | VLTVLHQDWLNGKEYKCKVSNKALPAPIEKTISKAKGQPREPQVCTLPPSR |
| | BH794_VL | EEMTKNQVSLSCAVKGFYPSDIAVEWESNGQPENNYKTTPPVLDSDGSFFL |
| | | VSKLTVDKSRWQQGNVFSCSVMHEALHNHYTQKSLSLSPGXGGGGSGGGGS |
| | | GGGGSQVQLVQSGAEVKKPGSSVKVSCKASGGTFSSYAISWVRQAPGQGLE |
| | | WMGGIIPIFGTANYAQKFQGRVTITADKSTSTAYMELSSLRSEDTAVYYCA |
| | | RGGYSYGSLDYWGQGTLVTVSSGGGGSGGGGSGGGGSDIQMTQSPSSLSAS |
| | | VGDRVTITCRASQSIISDLNWYQQKPGKAPKLLIHAASSLQSGVPSRFSGS |
| | | GSGTDFTLTISSLQPEDFATYYCQQSYSTPYTFGQGTKLEIK, wherein |
| | | X is K or absent |
| SEQ ID | aCSF1R | QVQLVQSGGGVVQPGRSLRLSCAASGFTFSSYAMHWVRQAPGKGLEWVAVI |
| NO: | BI123 VH- | SYDGSNKYYADSVKGRFTISRDNSKNTLYLQMNSLRAEDTAVYYCARELDY |
| 375 | hCH1- | GDYGLDVWGKGTTVTVSSASTKGPSVFPLAPSSKSTSGGTAALGCLVKDYF |
| | hFc_Hole- | PEPVTVSWNSGALTSGVHTFPAVLQSSGLYSLSSVVTVPSSSLGTQTYICN |
| | 3 x 4GS- | VNHKPSNTKVDKRVEPKSCDKTHTCPPCPAPELLGGPSVFLFPPKPKDTLM |
| | BH794_VH- | ISRTPEVTCVVVDVSHEDPEVKFNWYVDGVEVHNAKTKPREEQYNSTYRVV |
| | 3 x 4GS- | SVLTVLHQDWLNGKEYKCKVSNKALPAPIEKTISKAKGQPREPQVCTLPPS |
| | BH794_VL | REEMTKNQVSLSCAVKGFYPSDIAVEWESNGQPENNYKTTPPVLDSDGSFF |
| | | LVSKLTVDKSRWQQGNVFSCSVMHEALHNHYTQKSLSLSPGXGGGGSGGGG |
| | | SGGGGSQVQLVQSGAEVKKPGSSVKVSCKASGGTFSSYAISWVRQAPGQGL |
| | | EWMGGIIPIFGTANYAQKFQGRVTITADKSTSTAYMELSSLRSEDTAVYYC |
| | | ARGGYSYGSLDYWGQGTLVTVSSGGGGSGGGGSGGGGSDIQMTQSPSSLSA |
| | | SVGDRVTITCRASQSIISDLNWYQQKPGKAPKLLIHAASSLQSGVPSRFSG |
| | | SGSGTDFTLTISSLQPEDFATYYCQQSYSTPYTFGQGTKLEIK, |
| | | wherein X is K or absent |
| SEQ ID | aCCR2 | EVQLVESGGGLVKPGGSLRLSCAASGFSFNAYAMNWVRQAPGKGLEWVGRI |
| NO: | Hum1 VH- | RTKNNNYATYYADSVKDRFTISRDDSKSTLYLQMNSLKTEDTAVYYCTTFY |
| 376 | hCH1- | GNGVWGQGTTVTVSSASTKGPSVFPLAPSSKSTSGGTAALGCLVKDYFPEP |
| | hFc_Knob- | VTVSWNSGALTSGVHTFPAVLQSSGLYSLSSVVTVPSSSLGTQTYICNVNH |
| | 3 x 4GS- | KPSNTKVDKRVEPKSCDKTHTCPPCPAPELLGGPSVFLFPPKPKDTLMISR |
| | BH794_VH- | TPEVTCVVVDVSHEDPEVKFNWYVDGVEVHNAKTKPREEQYNSTYRVVSVL |
| | 3 x 4GS- | TVLHQDWLNGKEYKCKVSNKALPAPIEKTISKAKGQPREPQVYTLPPCREE |
| | BH794_VL | MTKNQVSLWCLVKGFYPSDIAVEWESNGQPENNYKTTPPVLDSDGSFFLYS |
| | | KLTVDKSRWQQGNVFSCSVMHEALHNHYTQKSLSLSPGXGGGGSGGGGSGG |
| | | GGSQVQLVQSGAEVKKPGSSVKVSCKASGGTFSSYAISWVRQAPGQGLEWM |
| | | GGIIPIFGTANYAQKFQGRVTITADKSTSTAYMELSSLRSEDTAVYYCARG |
| | | GYSYGSLDYWGQGTLVTVSSGGGGSGGGGSGGGGSDIQMTQSPSSLSASVG |
| | | DRVTITCRASQSIISDLNWYQQKPGKAPKLLIHAASSLQSGVPSRFSGSGS |
| | | GTDFTLTISSLQPEDFATYYCQQSYSTPYTFGQGTKLEIK, wherein X |
| | | is K or absent |
| SEQ ID | aCSF1R | QLVLTQPPSVSGAPGQRVTISCTGSSSNIGAGYDVHWYQQLPGTAPKLLIY |
| NO: | BI117 VL- | GNSNRPSGVPDRFSGSKSGTSASLAITGLQAEDEADYYCQSYDSSLSEEVF |
| 377 | hCLIg_v1- | GGGTKLTVLGQPKANPTVTLFPPSSEELQANKATLVCLISDFYPGAVTVAW |
| | 3 x 4GS-IL2 | KADGSPVKAGVETTKPSKQSNNKYAASSYLSLTPEQWKSHRSYSCQVTHEG |
| | | STVEKTVAPTECSGGGGSGGGGSGGGGSAPTSSSTKKTQLQLEHLLLDLQM |
| | | ILNGINNYKNPKLTRMLTFKFYMPKKATELKHLQCLEEELKPLEEVLNLAQ |
| | | SKNFHLRPRDLISNINVIVLELKGSETTFMCEYADETATIVEFLNRWITFC |
| | | QSIISTLT |
| SEQ ID | aCSF1R | QLVLTQPPSVSGAPGQRVTISCTGSSSNIGAGYDVHWYQQLPGTAPKLLIY |
| NO: | BI117 VL- | GNSNRPSGVPDRFSGSKSGTSASLAITGLQAEDEADYYCQSYDSSLSEEVF |
| 378 | hCLIg_v1- | GGGTKLTVLGQPKANPTVTLFPPSSEELQANKATLVCLISDFYPGAVTVAW |
| | IL2 | KADGSPVKAGVETTKPSKQSNNKYAASSYLSLTPEQWKSHRSYSCQVTHEG |
| | | STVEKTVAPTECSAPTSSSTKKTQLQLEHLLLDLQMILNGINNYKNPKLTR |
| | | MLTFKFYMPKKATELKHLQCLEEELKPLEEVLNLAQSKNFHLRPRDLISNI |
| | | NVIVLELKGSETTFMCEYADETATIVEFLNRWITFCQSIISTLT |
| SEQ ID | aCSF1R | SLVLTQPPSVSVAPGKTARITCGGNNIGSKSVHWYQQKPGQAPVLVVYDDS |
| NO: | BI123 VL- | DRPSGIPERFSGSNSGNTATLTISRVEAGDEADYYCQVWDSSSDHVVFGGG |
| 379 | hCLIg_v1- | TQLTVLGQPKANPTVTLFPPSSEELQANKATLVCLISDFYPGAVTVAWKAD |
| | 3 x 4GS-IL2 | GSPVKAGVETTKPSKQSNNKYAASSYLSLTPEQWKSHRSYSCQVTHEGSTV |
| | | EKTVAPTECSGGGGSGGGGSGGGGSAPTSSSTKKTQLQLEHLLLDLQMILN |
| | | GINNYKNPKLTRMLTFKFYMPKKATELKHLQCLEEELKPLEEVLNLAQSKN |
| | | FHLRPRDLISNINVIVLELKGSETTFMCEYADETATIVEFLNRWITFCQSI |
| | | ISTLT |
| SEQ ID | aCSF1R | SLVLTQPPSVSVAPGKTARITCGGNNIGSKSVHWYQQKPGQAPVLVVYDDS |
| NO: | BI123 VL- | DRPSGIPERFSGSNSGNTATLTISRVEAGDEADYYCQVWDSSSDHVVFGGG |
| 380 | hCLIg_v1- | TQLTVLGQPKANPTVTLFPPSSEELQANKATLVCLISDFYPGAVTVAWKAD |
| | IL2 | GSPVKAGVETTKPSKQSNNKYAASSYLSLTPEQWKSHRSYSCQVTHEGSTV |
| | | EKTVAPTECSAPTSSSTKKTQLQLEHLLLDLQMILNGINNYKNPKLTRMLT |
| | | FKFYMPKKATELKHLQCLEEELKPLEEVLNLAQSKNFHLRPRDLISNINVI |
| | | VLELKGSETTFMCEYADETATIVEFLNRWITFCQSIISTLT |
| SEQ ID | aCCR2 | DVVMTQSPLSLPVTLGQPASISCKSSQSLLDSDGKTFLNWLQQRPGQSPRR |
| NO: | VL- | LIYLVSKLDSGVPDRFSGSGSGTDFTLKISRVEAEDVGVYYCWQGTHFPYT |
| 381 | hCLIg_vk- | FGGGTKVEIKRTVAAPSVFIFPPSDEQLKSGTASVVCLLNNFYPREAKVQW |
| | 3 x 4GS-IL2 | KVDNALQSGNSQESVIEQDSKDSTYSLSSTLTLSKADYEKHKVYACEVTHQ |
| | | GLSSPVTKSFNRGECGGGGSGGGGSGGGGSAPTSSSTKKTQLQLEHLLLDL |
| | | QMILNGINNYKNPKLTRMLTFKFYMPKKATELKHLQCLEEELKPLEEVLNL |
| | | AQSKNFHLRPRDLISNINVIVLELKGSETTFMCEYADETATIVEFLNRWIT |
| | | FCQSIISTLT |
| SEQ ID | aCCR2 | DVVMTQSPLSLPVTLGQPASISCKSSQSLLDSDGKTFLNWLQQRPGQSPRR |
| NO: | VL- | LIYLVSKLDSGVPDRFSGSGSGTDFTLKISRVEAEDVGVYYCWQGTHFPYT |
| 382 | hCLIg_vk- | FGGGTKVEIKRTVAAPSVFIFPPSDEQLKSGTASVVCLLNNFYPREAKVQW |
| | IL2 | KVDNALQSGNSQESVIEQDSKDSTYSLSSTLTLSKADYEKHKVYACEVTHQ |
| | | GLSSPVTKSFNRGECAPTSSSTKKTQLQLEHLLLDLQMILNGINNYKNPKL |
| | | TRMLTFKFYMPKKATELKHLQCLEEELKPLEEVLNLAQSKNFHLRPRDLIS |
| | | NINVIVLELKGSETTFMCEYADETATIVEFLNRWITFCQSIISTLT |
| SEQ ID | aCSF1R | EVQLVESGGGLVQPGGSLRLSCAASGFTFSNDEMNWIRQAPGKGLEWVSYI |
| NO: | BI117 VH- | SSRSSTIYYADAVKGRFTISRDNARNSLYLQMNSLRAEDTAVYYCARGVDG |
| 383 | hCH1- | DFSFDYWGQGTLVTVSSASTKGPSVFPLAPSSKSTSGGTAALGCLVKDYFP |
| | hFc_Hole- | EPVTVSWNSGALTSGVHTFPAVLQSSGLYSLSSVVTVPSSSLGTQTYICNV |
| | 3 x 4GS- | NHKPSNTKVDKRVEPKSCDKTHTCPPCPAPELLGGPSVFLFPPKPKDTLMI |
| | TGFbR2 | SRTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVCTLPPSR |
| | | EEMTKNQVSLSCAVKGFYPSDIAVEWESNGQPENNYKTTPPVLDSDGSFFL |
| | | VSKLTVDKSRWQQGNVFSCSVMHEALHNHYTQKSLSLSPGXGGGGSGGGGS |
| | | GGGGSIPPHVQKSVNNDMIVTDNNGAVKFPQLCKFCDVRFSTCDNQKSCMS |
| | | NCSITSICEKPQEVCVAVWRKNDENITLETVCHDPKLPYHDFILEDAASPK |
| | | CIMKEKKKPGETFFMCSCSSDECNDNIIFSEEYNTSNPD, wherein X |
| | | is K or absent |
| SEQ ID | aCSF1R | QVQLVQSGGGVVQPGRSLRLSCAASGFTFSSYAMHWVRQAPGKGLEWVAVI |
| NO: | BI123 VH- | SYDGSNKYYADSVKGRFTISRDNSKNTLYLQMNSLRAEDTAVYYCARELDY |
| 384 | hCH 1- | GDYGLDVWGKGTTVTVSSASTKGPSVFPLAPSSKSTSGGTAALGCLVKDYF |
| | hFc_Hole- | PEPVTVSWNSGALTSGVHTFPAVLQSSGLYSLSSVVTVPSSSLGTQTYICN |
| | 3 x 4GS- | VNHKPSNTKVDKRVEPKSCDKTHTCPPCPAPELLGGPSVFLFPPKPKDTLM |
| | TGFbR2 | ISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVCTLPPS |
| | | REEMTKNQVSLSCAVKGFYPSDIAVEWESNGQPENNYKTTPPVLDSDGSFF |
| | | LVSKLTVDKSRWQQGNVFSCSVMHEALHNHYTQKSLSLSPGXGGGGSGGGG |
| | | SGGGGSIPPHVQKSVNNDMIVTDNNGAVKFPQLCKFCDVRFSTCDNQKSCM |
| | | SNCSITSICEKPQEVCVAVWRKNDENITLETVCHDPKLPYHDFILEDAASP |
| | | KCIMKEKKKPGETFFMCSCSSDECNDNIIFSEEYNTSNPD, wherein X |
| | | is K or absent |
| SEQ ID | aCCR2 | EVQLVESGGGLVKPGGSLRLSCAASGFSFNAYAMNWVRQAPGKGLEWVGRI |
| NO: | Hum1 VH- | RTKNNNYATYYADSVKDRFTISRDDSKSTLYLQMNSLKTEDTAVYYCTTFY |
| 385 | hCH 1- | GNGVWGQGTTVTVSSASTKGPSVFPLAPSSKSTSGGTAALGCLVKDYFPEP |
| | hFc_Knob- | VTVSWNSGALTSGVHTFPAVLQSSGLYSLSSVVTVPSSSLGTQTYICNVNH |
| | 3 x 4GS- | KPSNTKVDKRVEPKSCDKTHTCPPCPAPELLGGPSVFLFPPKPKDTLMISR |
| | TGFbR2 | TPEVTCVVVDVSHEDPEVKFNWYVDGVEVHNAKTKPREEQYNSTYRVVSVL |
| | | TVLHQDWLNGKEYKCKVSNKALPAPIEKTISKAKGQPREPQVYTLPPCREE |
| | | MTKNQVSLWCLVKGFYPSDIAVEWESNGQPENNYKTTPPVLDSDGSFFLYS |
| | | KLTVDKSRWQQGNVFSCSVMHEALHNHYTQKSLSLSPGXGGGGSGGGGSGG |
| | | GGSIPPHVQKSVNNDMIVTDNNGAVKFPQLCKFCDVRFSTCDNQKSCMSNC |
| | | SITSICEKPQEVCVAVWRKNDENITLETVCHDPKLPYHDFILEDAASPKCI |
| | | MKEKKKPGETFFMCSCSSDECNDNIIFSEEYNTSNPD, wherein X is |
| | | K or absent |
| SEQ ID | aCSF1R | EVQLVESGGGLVQPGGSLRLSCAASGFTFSNDEMNWIRQAPGKGLEWVSYI |
| NO: | BI117 VH- | SSRSSTIYYADAVKGRFTISRDNARNSLYLQMNSLRAEDTAVYYCARGVDG |
| 386 | 3 x 4GS- | DFSFDYWGQGTLVTVSSGGGGSGGGGSGGGGSQLVLTQPPSVSGAPGQRVT |
| | aCSF1R | ISCTGSSSNIGAGYDVHWYQQLPGTAPKLLIYGNSNRPSGVPDRFSGSKSG |
| | BI117 VL- | TSASLAITGLQAEDEADYYCQSYDSSLSEEVFGGGTKLTVLGGGGSDKTHT |
| | 4GS- | CPPCPAPELLGGPSVFLFPPKPKDTLMISRTPEVTCVVVDVSHEDPEVKFN |
| | hFc_Hole | WYVDGVEVHNAKTKPREEQYNSTYRVVSVLTVLHQDWLNGKEYKCKVSNKA |
| | | LPAPIEKTISKAKGQPREPQVCTLPPSREEMTKNQVSLSCAVKGFYPSDIA |
| | | VEWESNGQPENNYKTTPPVLDSDGSFFLVSKLTVDKSRWQQGNVFSCSVMH |
| | | EALHNHYTQKSLSLSPGX, wherein X is K or absent |
| SEQ ID | aCSF1R | QVQLVQSGGGVVQPGRSLRLSCAASGFTFSSYAMHWVRQAPGKGLEWVAVI |
| NO: | BI123 VH- | SYDGSNKYYADSVKGRFTISRDNSKNTLYLQMNSLRAEDTAVYYCARELDY |
| 387 | 3 x 4GS- | GDYGLDVWGKGTTVTVSSGGGGSGGGGSGGGGSSLVLTQPPSVSVAPGKTA |
| | aCSF1R | RITCGGNNIGSKSVHWYQQKPGQAPVLVVYDDSDRPSGIPERFSGSNSGNT |
| | BI123 VL- | ATLTISRVEAGDEADYYCQVWDSSSDHVVFGGGTQLTVLGGGGSDKTHTCP |
| | 4GS- | PCPAPELLGGPSVFLFPPKPKDTLMISRTPEVTCVVVDVSHEDPEVKFNWY |
| | hFc_Hole | VDGVEVHNAKTKPREEQYNSTYRVVSVLTVLHQDWLNGKEYKCKVSNKALP |
| | | APIEKTISKAKGQPREPQVCTLPPSREEMTKNQVSLSCAVKGFYPSDIAVE |
| | | WESNGQPENNYKTTPPVLDSDGSFFLVSKLTVDKSRWQQGNVFSCSVMHEA |
| | | LHNHYTQKSLSLSPGX, wherein X is K or absent |
| SEQ ID | aCSF1R | EVQLVESGGGLVQPGGSLRLSCAASGFTFSNDEMNWIRQAPGKGLEWVSYI |
| NO: | BI117 VH- | SSRSSTIYYADAVKGRFTISRDNARNSLYLQMNSLRAEDTAVYYCARGVDG |
| 388 | 3 x 4GS- | DFSFDYWGQGTLVTVSSGGGGSGGGGSGGGGSQLVLTQPPSVSGAPGQRVT |
| | aCSF1R | ISCTGSSSNIGAGYDVHWYQQLPGTAPKLLIYGNSNRPSGVPDRFSGSKSG |
| | BI117 VL- | TSASLAITGLQAEDEADYYCQSYDSSLSEEVFGGGTKLTVLGGGGSDKTHT |
| | 4GS- | CPPCPAPELLGGPSVFLFPPKPKDTLMISRTPEVTCVVVDVSHEDPEVKFN |
| | hFc_Hole- | WYVDGVEVHNAKTKPREEQYNSTYRVVSVLTVLHQDWLNGKEYKCKVSNKA |
| | 3 x 4GS- | LPAPIEKTISKAKGQPREPQVCTLPPSREEMTKNQVSLSCAVKGFYPSDIA |
| | TGFbR2 | VEWESNGQPENNYKTTPPVLDSDGSFFLVSKLTVDKSRWQQGNVFSCSVMH |
| | | EALHNHYTQKSLSLSPGXGGGGSGGGGSGGGGSIPPHVQKSVNNDMIVTDN |
| | | NGAVKFPQLCKFCDVRFSTCDNQKSCMSNCSITSICEKPQEVCVAVWRKND |
| | | ENITLETVCHDPKLPYHDFILEDAASPKCIMKEKKKPGETFFMCSCSSDEC |
| | | NDNIIFSEEYNTSNPD, wherein X is K or absent |
| SEQ ID | aCSF1R | QVQLVQSGGGVVQPGRSLRLSCAASGFTFSSYAMHWVRQAPGKGLEWVAVI |
| NO: | BI123 VH- | SYDGSNKYYADSVKGRFTISRDNSKNTLYLQMNSLRAEDTAVYYCARELDY |
| 389 | 3 x 4GS- | GDYGLDVWGKGTTVTVSSGGGGSGGGGSGGGGSSLVLTQPPSVSVAPGKTA |
| | aCSF1R | RITCGGNNIGSKSVHWYQQKPGQAPVLVVYDDSDRPSGIPERFSGSNSGNT |
| | BI123 VL- | ATLTISRVEAGDEADYYCQVWDSSSDHVVFGGGTQLTVLGGGGSDKTHTCP |
| | 4GS- | PCPAPELLGGPSVFLFPPKPKDTLMISRTPEVTCVVVDVSHEDPEVKFNWY |
| | hFc_Hole- | VDGVEVHNAKTKPREEQYNSTYRVVSVLTVLHQDWLNGKEYKCKVSNKALP |
| | 3 x 4GS- | APIEKTISKAKGQPREPQVCTLPPSREEMTKNQVSLSCAVKGFYPSDIAVE |
| | TGFbR2 | WESNGQPENNYKTTPPVLDSDGSFFLVSKLTVDKSRWQQGNVFSCSVMHEA |
| | | LHNHYTQKSLSLSPGXGGGGSGGGGSGGGGSIPPHVQKSVNNDMIVTDNNG |
| | | AVKFPQLCKFCDVRFSTCDNQKSCMSNCSITSICEKPQEVCVAVWRKNDEN |
| | | ITLETVCHDPKLPYHDFILEDAASPKCIMKEKKKPGETFFMCSCSSDECND |
| | | NIIFSEEYNTSNPD, wherein X is K or absent |
| SEQ ID | TGFbR2- | IPPHVQKSVNNDMIVTDNNGAVKFPQLCKFCDVRFSTCDNQKSCMSNCSIT |
| NO: | 3 x 4GS- | SICEKPQEVCVAVWRKNDENITLETVCHDPKLPYHDFILEDAASPKCIMKE |
| 390 | hCH1- | KKKPGETFFMCSCSSDECNDNIIFSEEYNTSNPDGGGGSGGGGSGGGGSAS |
| | hFc_Hole | TKGPSVFPLAPSSKSTSGGTAALGCLVKDYFPEPVTVSWNSGALTSGVHTF |
| | | PAVLQSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGKEYKCKV |
| | | SNKALPAPIEKTISKAKGQPREPQVCTLPPSREEMTKNQVSLSCAVKGFYP |
| | | SDIAVEWESNGQPENNYKTTPPVLDSDGSFFLVSKLTVDKSRWQQGNVFSC |
| | | SVMHEALHNHYTQKSLSLSPGX, wherein X is K or absent |
| SEQ ID | TGFbR2- | IPPHVQKSVNNDMIVTDNNGAVKFPQLCKFCDVRFSTCDNQKSCMSNCSIT |
| NO: | 3 x 4GS- | SICEKPQEVCVAVWRKNDENITLETVCHDPKLPYHDFILEDAASPKCIMKE |
| 391 | hCLIg_v1 | KKKPGETFFMCSCSSDECNDNIIFSEEYNTSNPDGGGGSGGGGSGGGGSGQ |
| | | LPKANPTVTFPPSSEELQANKATLVCLISDFYPGAVTVAWKADGSPVKAGV |
| | | ETTKPSKQSNNKYAASSYLSLTPEQWKSHRSYSCQVTHEGSTVEKTVAPTE |
| | | CS |
| SEQ ID | aCCR2 | EVQLVESGGGLVKPGGSLRLSCAASGFSFNAYAMNWVRQAPGKGLEWVGRI |
| NO: | Hum1 VH- | RTKNNNYATYYADSVKDRFTISRDDSKSTLYLQMNSLKTEDTAVYYCTTFY |
| 392 | hCH1- | GNGVWGQGTTVTVSSASTKGPSVFPLAPSSKSTSGGTAALGCLVKDYFPEP |
| | hFc_Knob- | VTVSWNSGALTSGVHTFPAVLQSSGLYSLSSVVTVPSSSLGTQTYICNVNH |
| | 3 x 4GS- | KPSNTKVDKRVEPKSCDKTHTCPPCPAPELLGGPSVFLFPPKPKDTLMISR |
| | BI117 VH- | TPEVTCVVVDVSHEDPEVKFNWYVDGVEVHNAKTKPREEQYNSTYRVVSVL |
| | 3 x 4GS- | TVLHQDWLNGKEYKCKVSNKALPAPIEKTISKAKGQPREPQVYTLPPCREE |
| | aCSF1R | MTKNQVSLWCLVKGFYPSDIAVEWESNGQPENNYKTTPPVLDSDGSFFLYS |
| | BI117 VL | KLTVDKSRWQQGNVFSCSVMHEALHNHYTQKSLSLSPGXGGGGSGGGGSGG |
| | | GGSEVQLVESGGGLVQPGGSLRLSCAASGFTFSNDEMNWIRQAPGKGLEWV |
| | | SYISSRSSTIYYADAVKGRFTISRDNARNSLYLQMNSLRAEDTAVYYCARG |
| | | VDGDFSFDYWGQGTLVTVSSGGGGSGGGGSGGGGSQLVLTQPPSVSGAPGQ |
| | | RVTISCTGSSSNIGAGYDVHWYQQLPGTAPKLLIYGNSNRPSGVPDRFSGS |
| | | KSGTSASLAITGLQAEDEADYYCQSYDSSLSEEVFGGGTKLTVL, |
| | | wherein X is K or absent |
| SEQ ID | aCCR2 | EVQLVESGGGLVQPGGSLRLSCAASGFSFNAYAMNWVRQAPGKGLEWVGRI |
| NO: | Hum1 VH- | RTKNNNYATYYADSVKDRFTISRDDSKSSLYLQMNSLK1EDTAVYYCATFY |
| 393 | hCH1- | GNGVWGQGTTVTVSSASTKGPSVFPLAPSSKSTSGGTAALGCLVKDYFPEP |
| | hFc_Knob- | VTVSWNSGALTSGVHTFPAVLQSSGLYSLSSVVTVPSSSLGTQTYICNVNH |
| | 3 x 4GS- | KPSNTKVDKRVEPKSCDKTHTCPPCPAPELLGGPSVFLFPPKPKDTLMISR |
| | BI123 VH- | TPEVTCVVVDVSHEDPEVKFNWYVDGVEVHNAKTKPREEQYNSTYRVVSVL |
| | 3 x 4GS- | TVLHQDWLNGKEYKCKVSNKALPAPIEKTISKAKGQPREPQVYTLPPCREE |
| | aCSF1R | MTKNQVSLWCLVKGFYPSDIAVEWESNGQPENNYKTTPPVLDSDGSFFLYS |
| | BI123 VL | KLTVDKSRWQQGNVFSCSVMHEALHNHYTQKSLSLSPGXGGGGSGGGGSGG |
| | | GGSQVQLVQSGGGVVQPGRSLRLSCAASGFTFSSYAMHWVRQAPGKGLEWV |
| | | AVISYDGSNKYYADSVKGRFTISRDNSKNTLYLQMNSLRAEDTAVYYCARE |
| | | LDYGDYGLDVWGKGTTVTVSSGGGGSGGGGSGGGGSSLVLTQPPSVSVAPG |
| | | KTARITCGGNNIGSKSVHWYQQKPGQAPVLVVYDDSDRPSGIPERFSGSNS |
| | | GNTATLTISRVEAGDEADYYCQVWDSSSDHVVFGGGTQLTVL, wherein |
| | | X is K or absent |
| SEQ ID | hFc-Knob | DKTHTCPPCPAPELLGGPSVFLFPPKPKDTLMISRTPEVTCVVVDVSHEDP |
| NO: | (H435R, | EVKFNWYVDGVEVHNAKTKPREEQYNSTYRVVSVLTVLHQDWLNGKEYKCK |
| 401 | Y436F)_his | VSNKALPAPIEKTISKAKGQPREPQVYTLPPCREEMTKNQVSLWCLVKGFY |
| | tag | PSDIAVEWESNGQPENNYKTTPPVLDSDGSFFLYSKLTVDKSRWQQGNVFS |
| | | CSVMHEALHNRFTQKSLSLSPGKGGGGSGGGGSHHHHHHHH |
| SEQ ID | hTGFbR2 | MGRGLLRGLWPLHIVLWTRIAST |
| NO: | signal | |
| 348 | peptide | |
| SEQ ID | 3 × 4GS | GGGGSGGGGSGGGGS |
| NO: | linker | |
| 349 | | |
| SEQ ID | 4GS linker | GGGGS |
| NO: | | |
| 350 | | |
| SEQ ID | 2 × 4GS | GGGGSGGGGS |
| NO: | linker | |
| 398 | | |
| SEQ ID | hFc_Knob | DKTHTCPPCPAPELLGGPSVFLFPPKPKDTLMISRTPEVTCVVVDVSHEDP |
| NO: | (H435R, | EVKFNWYVDGVEVHNAKTKPREEQYNSTYRVVSVLTVLHQDWLNGKEYKCK |
| 399 | Y436F) | VSNKALPAPIEKTISKAKGQPREPQVYTLPPCREEMTKNQVSLWCLVKGFY |
| | | PSDIAVEWESNGQPENNYKTTPPVLDSDGSFFLYSKLTVDKSRWQQGNVFS |
| | | CSVMHEALHNRFTQKSLSLSPGK |
| SEQ ID | 8 × His | HHHHHHHH |
| NO: | | |
| 400 | | |
| SEQ ID | aCSF1R | ATGGAAACCGACACACTGCTGCTGTGGGTGCTGCTCTTGTGGGTGCCAGGA |
| NO: | BI117 VH- | TCTACAGGAGAAGTGCAGCTGGTCGAAAGCGGAGGGGGTCTGGTCCAGCCT |
| 280 | hCH1- | GGAGGGTCCCTGCGGCTGTCCTGCGCCGCTTCCGGCTTCACCTTCTCCAAC |
| | hFc_Hole | GATGAAATGAACTGGATCCGCCAAGCCCCTGGAAAGGGCCTGGAATGGGTG |
| | | TCGTACATCTCGAGCAGGTCCAGCACTATCTACTATGCCGACGCGGTGAAG |
| | | GGACGCTTCACTATCAGCCGCGATAACGCACGCAATTCCCTCTACCTGCAG |
| | | ATGAACTCGCTCAGAGCTGAGGATACTGCCGTCTACTACTGCGCCCGCGGC |
| | | GTGGACGGCGATTTTTCCTTCGATTACTGGGGACAGGGCACTCTTGTCACC |
| | | GTGTCAAGCGCTTCTACCAAGGGACCCAGCGTGTTCCCTCTGGCTCCTTCC |
| | | AGCAAGTCTACCTCTGGCGGAACAGCTGCTCTGGGCTGCCTGGTCAAGGAC |
| | | TACTTTCCTGAGCCTGTGACCGTGTCTTGGAACTCTGGCGCTCTGACATCC |
| | | GGCGTGCACACATTTCCAGCTGTGCTGCAGTCCTCCGGCCTGTACTCTCTG |
| | | TCCTCTGTCGTGACCGTGCCTTCCAGCTCTCTGGGAACCCAGACCTACATC |
| | | TGCAATGTGAACCACAAGCCTTCCAACACCAAGGTGGACAAGAGAGTGGAA |
| | | CCCAAGTCCTGCGATAAGACCCACACCTGTCCTCCCTGCCCTGCCCCTGAA |
| | | CTGCTGGGCGGACCTAGCGTGTTCCTGTTCCCTCCAAAGCCCAAGGACACC |
| | | CTGATGATCAGCCGGACCCCTGAAGTGACCTGCGTGGTGGTGGATGTGTCC |
| | | CACGAGGATCCCGAAGTGAAGTTCAATTGGTACGTGGACGGCGTGGAAGTG |
| | | CACAACGCCAAGACCAAGCCCAGAGAGGAACAGTACAACAGCACCTACCGG |
| | | GTGGTGTCCGTGCTGACCGTGCTGCACCAGGACTGGCTGAACGGCAAAGAG |
| | | TACAAGTGCAAGGTGTCCAACAAGGCCCTGCCAGCCCCTATCGAGAAAACC |
| | | ATCAGCAAGGCCAAGGGCCAGCCTAGAGAGCCTCAGGTCTGCACCCTGCCT |
| | | CCCAGCCGGGAAGAGATGACCAAGAACCAGGTGTCCCTGAGCTGCGCCGTG |
| | | AAGGGCTTCTACCCCTCCGATATCGCCGTGGAATGGGAGAGCAACGGCCAG |
| | | CCCGAGAACAACTACAAGACCACCCCTCCCGTGCTGGACAGCGACGGCAGC |
| | | TTCTTCCTGGTGTCCAAACTGACCGTGGACAAGAGCCGGTGGCAGCAGGGC |
| | | AATGTGTTCAGCTGTAGCGTGATGCACGAGGCCCTGCACAACCACTACACC |
| | | CAGAAGTCTCTGAGCCTGAGCCCTGGCAAA |
| SEQ ID | aCSF1R | ATGGAAACCGACACACTGCTGCTGTGGGTGCTGCTCTTGTGGGTGCCAGGA |
| NO: | BI117 C5- | TCTACAGGAGAAGTCCAACTCGTGGAAAGCGGCGGTGGCCTGGTGCAGCCG |
| 281 | R2A VH- | GGAGGCTCCCTCCGGCTGTCGTGTGCAGCCAGCGGGTTCACCTTCTCCAAC |
| | hCH1- | GACGAAATGAACTGGATTCGCCAGGCGCCGGGAAAGGGGCTGGAATGGGTG |
| | hFc_Hole | TCGTACATTTCAAGCGGGTCCTCCACCATTTACTACGCCGATGCAGTCAAG |
| | | GGCCGCTTCACCATCTCGCGCGACAACGCTCGGAACTCCCTCTATCTGCAG |
| | | ATGAACAGCCTGAGAGCGGAAGACACCGCCGTCTACTACTGCGCCCGCGGA |
| | | GTGGACGGCGACTTCTCCTTCGACTACTGGGGTCAGGGAACCCTGGTGACT |
| | | GTGTCCTCAGCTTCTACCAAGGGACCCAGCGTGTTCCCTCTGGCTCCTTCC |
| | | AGCAAGTCTACCTCTGGCGGAACAGCTGCTCTGGGCTGCCTGGTCAAGGAC |
| | | TACTTTCCTGAGCCTGTGACCGTGTCTTGGAACTCTGGCGCTCTGACATCC |
| | | GGCGTGCACACATTTCCAGCTGTGCTGCAGTCCTCCGGCCTGTACTCTCTG |
| | | TCCTCTGTCGTGACCGTGCCTTCCAGCTCTCTGGGAACCCAGACCTACATC |
| | | TGCAATGTGAACCACAAGCCTTCCAACACCAAGGTGGACAAGAGAGTGGAA |
| | | CCCAAGTCCTGCGATAAGACCCACACCTGTCCTCCCTGCCCTGCCCCTGAA |
| | | CTGCTGGGCGGACCTAGCGTGTTCCTGTTCCCTCCAAAGCCCAAGGACACC |
| | | CTGATGATCAGCCGGACCCCTGAAGTGACCTGCGTGGTGGTGGATGTGTCC |
| | | CACGAGGATCCCGAAGTGAAGTTCAATTGGTACGTGGACGGCGTGGAAGTG |
| | | CACAACGCCAAGACCAAGCCCAGAGAGGAACAGTACAACAGCACCTACCGG |
| | | GTGGTGTCCGTGCTGACCGTGCTGCACCAGGACTGGCTGAACGGCAAAGAG |
| | | TACAAGTGCAAGGTGTCCAACAAGGCCCTGCCAGCCCCTATCGAGAAAACC |
| | | ATCAGCAAGGCCAAGGGCCAGCCTAGAGAGCCTCAGGTCTGCACCCTGCCT |
| | | CCCAGCCGGGAAGAGATGACCAAGAACCAGGTGTCCCTGAGCTGCGCCGTG |
| | | AAGGGCTTCTACCCCTCCGATATCGCCGTGGAATGGGAGAGCAACGGCCAG |
| | | CCCGAGAACAACTACAAGACCACCCCTCCCGTGCTGGACAGCGACGGCAGC |
| | | TTCTTCCTGGTGTCCAAACTGACCGTGGACAAGAGCCGGTGGCAGCAGGGC |
| | | AATGTGTTCAGCTGTAGCGTGATGCACGAGGCCCTGCACAACCACTACACC |
| | | CAGAAGTCTCTGAGCCTGAGCCCTGGCAAA |
| SEQ ID | aCSF1R | ATGGAAACCGACACACTGCTGCTGTGGGTGCTGCTCTTGTGGGTGCCAGGA |
| NO: | BI117 C9- | TCTACAGGAGAGGTGCAGCTGGTGGAATCGGGGGGTGGCCTCGTGCAGCCC |
| 282 | R2A VH- | GGAGGGTCGCTCCGGCTGTCCTGTGCCGCTAGCGGATTCACATTCTCGAAT |
| | hCH1- | GACGAGATGAACTGGATTCGGCAGGCCCCAGGCAAGGGCCTGGAATGGGTG |
| | hFc_Hole | AGCTTCATTTCGGGCGGGTCCTCAACCATCTACTACGCCGACGCCGTCAAG |
| | | GGGCGCTTTACTATCTCCCGCGACAATGCACGGAACTCCCTCTACCTGCAA |
| | | ATGAACAGCCTGAGGGCGGAAGATACAGCTGTGTACTACTGCGCCCGGGGC |
| | | GTGGACGGAGACTTCTCCTTCGACTACTGGGGTCAGGGAACCCTGGTGACA |
| | | GTGTCTTCCGCTTCTACCAAGGGACCCAGCGTGTTCCCTCTGGCTCCTTCC |
| | | AGCAAGTCTACCTCTGGCGGAACAGCTGCTCTGGGCTGCCTGGTCAAGGAC |
| | | TACTTTCCTGAGCCTGTGACCGTGTCTTGGAACTCTGGCGCTCTGACATCC |
| | | TGGCGGCACACATTTCCAGCTGTGCTGCAGTCCTCCGGCCTGTACTCTCTG |
| | | TCCTCTGTCGTGACCGTGCCTTCCAGCTCTCTGGGAACCCAGACCTACATC |
| | | TGCAATGTGAACCACAAGCCTTCCAACACCAAGGTGGACAAGAGAGTGGAA |
| | | CCCAAGTCCTGCGATAAGACCCACACCTGTCCTCCCTGCCCTGCCCCTGAA |
| | | CTGCTGGGCGGACCTAGCGTGTTCCTGTTCCCTCCAAAGCCCAAGGACACC |
| | | CTGATGATCAGCCGGACCCCTGAAGTGACCTGCGTGGTGGTGGATGTGTCC |
| | | CACGAGGATCCCGAAGTGAAGTTCAATTGGTACGTGGACGGCGTGGAAGTG |
| | | CACAACGCCAAGACCAAGCCCAGAGAGGAACAGTACAACAGCACCTACCGG |
| | | GTGGTGTCCGTGCTGACCGTGCTGCACCAGGACTGGCTGAACGGCAAAGAG |
| | | TACAAGTGCAAGGTGTCCAACAAGGCCCTGCCAGCCCCTATCGAGAAAACC |
| | | ATCAGCAAGGCCAAGGGCCAGCCTAGAGAGCCTCAGGTCTGCACCCTGCCT |
| | | CCCAGCCGGGAAGAGATGACCAAGAACCAGGTGTCCCTGAGCTGCGCCGTG |
| | | AAGGGCTTCTACCCCTCCGATATCGCCGTGGAATGGGAGAGCAACGGCCAG |
| | | CCCGAGAACAACTACAAGACCACCCCTCCCGTGCTGGACAGCGACGGCAGC |
| | | TTCTTCCTGGTGTCCAAACTGACCGTGGACAAGAGCCGGTGGCAGCAGGGC |
| | | AATGTGTTCAGCTGTAGCGTGATGCACGAGGCCCTGCACAACCACTACACC |
| | | CAGAAGTCTCTGAGCCTGAGCCCTGGCAAA |
| SEQ ID | aCSF1R | ATGGAAACCGACACACTGCTGCTGTGGGTGCTGCTCTTGTGGGTGCCAGGA |
| NO: | BI117 D1- | TCTACAGGAGAAGTGCAGCTCGTGGAGTCTGGAGGTGGTCTGGTGCAGCCG |
| 283 | R2A VH- | GGTGGTTCCCTGAGGCTGTCCTGCGCCGCCAGCGGCTTCACCTTCTCAAAT |
| | hCH1- | GACGAAATGAACTGGATTCGGCAGGCCCCTGGCAAGGGCCTGGAGTGGGTG |
| | hFc_Hole | TCGTACATCTCCTCCCGCAGCTCCACCATCTACTACGCCGACGCAGTCAAG |
| | | GGGAGGTTCACCATCTCCCGGGACAACGCCCGGAACTCACTGTACCTGCAG |
| | | ATGAACTCCCTGAGAGCTGAGGACACTGCCGTCTACTACTGCGCTAGAAAC |
| | | TCCGATGAGGACGGAAGCTTCGACTACTGGGGCCAGGGAACTCTGGTGACA |
| | | GTCTCGAGCGCTTCTACCAAGGGACCCAGCGTGTTCCCTCTGGCTCCTTCC |
| | | AGCAAGTCTACCTCTGGCGGAACAGCTGCTCTGGGCTGCCTGGTCAAGGAC |
| | | TACTTTCCTGAGCCTGTGACCGTGTCTTGGAACTCTGGCGCTCTGACATCC |
| | | GGCGTGCACACATTTCCAGCTGTGCTGCAGTCCTCCGGCCTGTACTCTCTG |
| | | TCCTCTGTCGTGACCGTGCCTTCCAGCTCTCTGGGAACCCAGACCTACATC |
| | | TGCAATGTGAACCACAAGCCTTCCAACACCAAGGTGGACAAGAGAGTGGAA |
| | | CCCAAGTCCTGCGATAAGACCCACACCTGTCCTCCCTGCCCTGCCCCTGAA |
| | | CTGCTGGGCGGACCTAGCGTGTTCCTGTTCCCTCCAAAGCCCAAGGACACC |
| | | CTGATGATCAGCCGGACCCCTGAAGTGACCTGCGTGGTGGTGGATGTGTCC |
| | | CACGAGGATCCCGAAGTGAAGTTCAATTGGTACGTGGACGGCGTGGAAGTG |
| | | CACAACGCCAAGACCAAGCCCAGAGAGGAACAGTACAACAGCACCTACCGG |
| | | GTGGTGTCCGTGCTGACCGTGCTGCACCAGGACTGGCTGAACGGCAAAGAG |
| | | TACAAGTGCAAGGTGTCCAACAAGGCCCTGCCAGCCCCTATCGAGAAAACC |
| | | ATCAGCAAGGCCAAGGGCCAGCCTAGAGAGCCTCAGGTCTGCACCCTGCCT |
| | | CCCAGCCGGGAAGAGATGACCAAGAACCAGGTGTCCCTGAGCTGCGCCGTG |
| | | AAGGGCTTCTACCCCTCCGATATCGCCGTGGAATGGGAGAGCAACGGCCAG |
| | | CCCGAGAACAACTACAAGACCACCCCTCCCGTGCTGGACAGCGACGGCAGC |
| | | TTCTTCCTGGTGTCCAAACTGACCGTGGACAAGAGCCGGTGGCAGCAGGGC |
| | | AATGTGTTCAGCTGTAGCGTGATGCACGAGGCCCTGCACAACCACTACACC |
| | | CAGAAGTCTCTGAGCCTGAGCCCTGGCAAA |
| SEQ ID | aCSF1R | ATGGAAACCGACACACTGCTGCTGTGGGTGCTGCTCTTGTGGGTGCCAGGA |
| NO: | BI117 E2- | TCTACAGGAGAGGTGCAGCTCGTGGAAAGCGGGGGCGGCCTGGTCCAGCCT |
| 284 | R2B VH- | GGAGGATCCCTGAGACTTTCCTGTGCTGCTTCCGGGTTCACCTTCAGCAAT |
| | hCH1- | GATGAGATGAACTGGATTCGGCAAGCCCCGGGCAAGGGCCTGGAATGGGTC |
| | hFc_Hole | AGCTACATCACGTCTAAGTCCTCCACCATCTACTATGCCGACTCCGTGAAG |
| | | GGACGCTTTACCATCTCACGCGACAACGCGCGGAACAGCCTTTACCTGCAG |
| | | ATGAACTCACTGCGCGCCGAAGATACCGCCGTGTACTACTGCGCTCGCGGA |
| | | GTGGATGGAGATTTTTCCTTCGATTACTGGGGACAAGGCACCCTGGTGACC |
| | | GTCAGCTCCGCTTCTACCAAGGGACCCAGCGTGTTCCCTCTGGCTCCTTCC |
| | | AGCAAGTCTACCTCTGGCGGAACAGCTGCTCTGGGCTGCCTGGTCAAGGAC |
| | | TACTTTCCTGAGCCTGTGACCGTGTCTTGGAACTCTGGCGCTCTGACATCC |
| | | GGCGTGCACACATTTCCAGCTGTGCTGCAGTCCTCCGGCCTGTACTCTCTG |
| | | TCCTCTGTCGTGACCGTGCCTTCCAGCTCTCTGGGAACCCAGACCTACATC |
| | | TGCAATGTGAACCACAAGCCTTCCAACACCAAGGTGGACAAGAGAGTGGAA |
| | | CCCAAGTCCTGCGATAAGACCCACACCTGTCCTCCCTGCCCTGCCCCTGAA |
| | | CTGCTGGGCGGACCTAGCGTGTTCCTGTTCCCTCCAAAGCCCAAGGACACC |
| | | CTGATGATCAGCCGGACCCCTGAAGTGACCTGCGTGGTGGTGGATGTGTCC |
| | | CACGAGGATCCCGAAGTGAAGTTCAATTGGTACGTGGACGGCGTGGAAGTG |
| | | CACAACGCCAAGACCAAGCCCAGAGAGGAACAGTACAACAGCACCTACCGG |
| | | GTGGTGTCCGTGCTGACCGTGCTGCACCAGGACTGGCTGAACGGCAAAGAG |
| | | TACAAGTGCAAGGTGTCCAACAAGGCCCTGCCAGCCCCTATCGAGAAAACC |
| | | ATCAGCAAGGCCAAGGGCCAGCCTAGAGAGCCTCAGGTCTGCACCCTGCCT |
| | | CCCAGCCGGGAAGAGATGACCAAGAACCAGGTGTCCCTGAGCTGCGCCGTG |
| | | AAGGGCTTCTACCCCTCCGATATCGCCGTGGAATGGGAGAGCAACGGCCAG |
| | | CCCGAGAACAACTACAAGACCACCCCTCCCGTGCTGGACAGCGACGGCAGC |
| | | TTCTTCCTGGTGTCCAAACTGACCGTGGACAAGAGCCGGTGGCAGCAGGGC |
| | | AATGTGTTCAGCTGTAGCGTGATGCACGAGGCCCTGCACAACCACTACACC |
| | | CAGAAGTCTCTGAGCCTGAGCCCTGGCAAA |
| SEQ ID | aCSF1R | ATGGAAACCGACACACTGCTGCTGTGGGTGCTGCTCTTGTGGGTGCCAGGA |
| NO: | BI117 G1- | TCTACAGGAGAAGTCCAGCTCGTCGAATCTGGTGGGGGACTGGTCCAGCCG |
| 285 | R2B VH- | GGAGGATCCCTCCGGCTGTCGTGCGCGGCATCCGGGTTCACCTTCTCCAAC |
| | hCH1- | GATGAGATGAATTGGATTCGCCAGGCCCCAGGAAAGGGGCTGGAGTGGGTC |
| | hFc_Hole | TCGTACATCTCATCCCGCAGCTCTACTATCTACTACGCCGATGCTGTCAAG |
| | | GGCCGGTTCACTATCTCCCGCGATAACGCCCGGAACTCCCTCTACCTCCAG |
| | | ATGAACTCACTGCGCGCGGAAGACACCGCCGTCTACTACTGCGCACGGGGC |
| | | GTCGATGGAGATTTCTCCTTCGATGTGTGGGGTCAGGGAACACTGGTGACC |
| | | GTGTCCTCCGCTTCTACCAAGGGACCCAGCGTGTTCCCTCTGGCTCCTTCC |
| | | AGCAAGTCTACCTCTGGCGGAACAGCTGCTCTGGGCTGCCTGGTCAAGGAC |
| | | TACTTTCCTGAGCCTGTGACCGTGTCTTGGAACTCTGGCGCTCTGACATCC |
| | | GGCGTGCACACATTTCCAGCTGTGCTGCAGTCCTCCGGCCTGTACTCTCTG |
| | | TCCTCTGTCGTGACCGTGCCTTCCAGCTCTCTGGGAACCCAGACCTACATC |
| | | TGCAATGTGAACCACAAGCCTTCCAACACCAAGGTGGACAAGAGAGTGGAA |
| | | CCCAAGTCCTGCGATAAGACCCACACCTGTCCTCCCTGCCCTGCCCCTGAA |
| | | CTGCTGGGCGGACCTAGCGTGTTCCTGTTCCCTCCAAAGCCCAAGGACACC |
| | | CTGATGATCAGCCGGACCCCTGAAGTGACCTGCGTGGTGGTGGATGTGTCC |
| | | CACGAGGATCCCGAAGTGAAGTTCAATTGGTACGTGGACGGCGTGGAAGTG |
| | | CACAACGCCAAGACCAAGCCCAGAGAGGAACAGTACAACAGCACCTACCGG |
| | | GTGGTGTCCGTGCTGACCGTGCTGCACCAGGACTGGCTGAACGGCAAAGAG |
| | | TACAAGTGCAAGGTGTCCAACAAGGCCCTGCCAGCCCCTATCGAGAAAACC |
| | | ATCAGCAAGGCCAAGGGCCAGCCTAGAGAGCCTCAGGTCTGCACCCTGCCT |
| | | CCCAGCCGGGAAGAGATGACCAAGAACCAGGTGTCCCTGAGCTGCGCCGTG |
| | | AAGGGCTTCTACCCCTCCGATATCGCCGTGGAATGGGAGAGCAACGGCCAG |
| | | CCCGAGAACAACTACAAGACCACCCCTCCCGTGCTGGACAGCGACGGCAGC |
| | | TTCTTCCTGGTGTCCAAACTGACCGTGGACAAGAGCCGGTGGCAGCAGGGC |
| | | AATGTGTTCAGCTGTAGCGTGATGCACGAGGCCCTGCACAACCACTACACC |
| | | CAGAAGTCTCTGAGCCTGAGCCCTGGCAAA |
| SEQ ID | aCSF1R | ATGGAAACCGACACACTGCTGCTGTGGGTGCTGCTCTTGTGGGTGCCAGGA |
| NO: | BI117 | TCTACAGGAGAGGTGCAGCTCGTGGAGTCCGGTGGAGGACTGGTGCAGCCC |
| 286 | G12-R2B | GGCGGATCGCTGAGACTGTCTTGCGCCGCCTCGGGATTCACGTTCTCCAAC |
| | VH-hCH1- | GATGAAATGAACTGGATTAGACAGGCTCCGGGCAAGGGCCTGGAATGGGTG |
| | hFc_Hole | TCCTACATTACTTCGAAGTCCTCCACCATTTACTACGCAGACGCCGTGAAG |
| | | GGCCGCTTCACCATTTCCCGCGACAACGCAAGAAACTCGCTGTACCTGCAG |
| | | ATGAACTCCCTGCGCGCGGAGGACACCGCCGTGTACTACTGTGCCAGAGGA |
| | | GTCGATGGCGATTTCTCGTTCGACTACTGGGGACAGGGCACTCTTGTGACT |
| | | GTCTCCTCCGCTTCTACCAAGGGACCCAGCGTGTTCCCTCTGGCTCCTTCC |
| | | AGCAAGTCTACCTCTGGCGGAACAGCTGCTCTGGGCTGCCTGGTCAAGGAC |
| | | TACTTTCCTGAGCCTGTGACCGTGTCTTGGAACTCTGGCGCTCTGACATCC |
| | | GGCGTGCACACATTTCCAGCTGTGCTGCAGTCCTCCGGCCTGTACTCTCTG |
| | | TCCTCTGTCGTGACCGTGCCTTCCAGCTCTCTGGGAACCCAGACCTACATC |
| | | TGCAATGTGAACCACAAGCCTTCCAACACCAAGGTGGACAAGAGAGTGGAA |
| | | CCCAAGTCCTGCGATAAGACCCACACCTGTCCTCCCTGCCCTGCCCCTGAA |
| | | CTGCTGGGCGGACCTAGCGTGTTCCTGTTCCCTCCAAAGCCCAAGGACACC |
| | | CTGATGATCAGCCGGACCCCTGAAGTGACCTGCGTGGTGGTGGATGTGTCC |
| | | CACGAGGATCCCGAAGTGAAGTTCAATTGGTACGTGGACGGCGTGGAAGTG |
| | | CACAACGCCAAGACCAAGCCCAGAGAGGAACAGTACAACAGCACCTACCGG |
| | | GTGGTGTCCGTGCTGACCGTGCTGCACCAGGACTGGCTGAACGGCAAAGAG |
| | | TACAAGTGCAAGGTGTCCAACAAGGCCCTGCCAGCCCCTATCGAGAAAACC |
| | | ATCAGCAAGGCCAAGGGCCAGCCTAGAGAGCCTCAGGTCTGCACCCTGCCT |
| | | CCCAGCCGGGAAGAGATGACCAAGAACCAGGTGTCCCTGAGCTGCGCCGTG |
| | | AAGGGCTTCTACCCCTCCGATATCGCCGTGGAATGGGAGAGCAACGGCCAG |
| | | CCCGAGAACAACTACAAGACCACCCCTCCCGTGCTGGACAGCGACGGCAGC |
| | | TTCTTCCTGGTGTCCAAACTGACCGTGGACAAGAGCCGGTGGCAGCAGGGC |
| | | AATGTGTTCAGCTGTAGCGTGATGCACGAGGCCCTGCACAACCACTACACC |
| | | CAGAAGTCTCTGAGCCTGAGCCCTGGCAAA |
| SEQ ID | aCSF1R | ATGGAAACCGACACACTGCTGCTGTGGGTGCTGCTCTTGTGGGTGCCAGGA |
| NO: | BI117 D6- | TCTACAGGAGAGGTCCAGCTGGTCGAATCGGGGGGAGGCCTCGTGCAGCCT |
| 287 | R3B VH- | GGAGGGTCCCTCCGGCTTTCCTGTGCCGCTTCCGGATTCACCTTCAGCAAC |
| | hCH1- | GATGAGATGAACTGGATCAGGCAGGCCCCGGGAAAGGGGCTCGAGTGGGTG |
| | hFc_Hole | TCCTACATCTCCAGCCGGTCCTCGACCATCTACTACGCCGATGCCGTGAAG |
| | | GGGAGATTTACTATCTCCCGCGACAATGCACGGAACTCACTGTACCTCCAG |
| | | ATGAACTCACTGCGGGCCGAGGACACCGCAGTGTACTACTGCGCGCGGTCC |
| | | GTGGATGGCGACGGGAGCTTCGATTACTGGGGACAGGGGACCCTGGTGACC |
| | | GTGTCGTCCGCTTCTACCAAGGGACCCAGCGTGTTCCCTCTGGCTCCTTCC |
| | | AGCAAGTCTACCTCTGGCGGAACAGCTGCTCTGGGCTGCCTGGTCAAGGAC |
| | | TACTTTCCTGAGCCTGTGACCGTGTCTTGGAACTCTGGCGCTCTGACATCC |
| | | GGCGTGCACACATTTCCAGCTGTGCTGCAGTCCTCCGGCCTGTACTCTCTG |
| | | TCCTCTGTCGTGACCGTGCCTTCCAGCTCTCTGGGAACCCAGACCTACATC |
| | | TGCAATGTGAACCACAAGCCTTCCAACACCAAGGTGGACAAGAGAGTGGAA |
| | | CCCAAGTCCTGCGATAAGACCCACACCTGTCCTCCCTGCCCTGCCCCTGAA |
| | | CTGCTGGGCGGACCTAGCGTGTTCCTGTTCCCTCCAAAGCCCAAGGACACC |
| | | CTGATGATCAGCCGGACCCCTGAAGTGACCTGCGTGGTGGTGGATGTGTCC |
| | | CACGAGGATCCCGAAGTGAAGTTCAATTGGTACGTGGACGGCGTGGAAGTG |
| | | CACAACGCCAAGACCAAGCCCAGAGAGGAACAGTACAACAGCACCTACCGG |
| | | GTGGTGTCCGTGCTGACCGTGCTGCACCAGGACTGGCTGAACGGCAAAGAG |
| | | TACAAGTGCAAGGTGTCCAACAAGGCCCTGCCAGCCCCTATCGAGAAAACC |
| | | ATCAGCAAGGCCAAGGGCCAGCCTAGAGAGCCTCAGGTCTGCACCCTGCCT |
| | | CCCAGCCGGGAAGAGATGACCAAGAACCAGGTGTCCCTGAGCTGCGCCGTG |
| | | AAGGGCTTCTACCCCTCCGATATCGCCGTGGAATGGGAGAGCAACGGCCAG |
| | | CCCGAGAACAACTACAAGACCACCCCTCCCGTGCTGGACAGCGACGGCAGC |
| | | TTCTTCCTGGTGTCCAAACTGACCGTGGACAAGAGCCGGTGGCAGCAGGGC |
| | | AATGTGTTCAGCTGTAGCGTGATGCACGAGGCCCTGCACAACCACTACACC |
| | | CAGAAGTCTCTGAGCCTGAGCCCTGGCAAA |
| SEQ ID | aCSF1R | ATGGAAACCGACACACTGCTGCTGTGGGTGCTGCTCTTGTGGGTGCCAGGA |
| NO: | BI117 A6- | TCTACAGGAGAGGTGCAGCTCGTGGAATCCGGAGGTGGACTGGTGCAGCCG |
| 288 | R3B VH- | GGGGGTAGCCTGCGGCTGTCCTGTGCAGCTTCCGGGTTCACCTTCTCCAAC |
| | hCH1- | GACGAAATGAACTGGATCCGCCAGGCCCCGGGCAAGGGACTCGAATGGGTG |
| | hFc_Hole | TCCTACATCTCCTCCCGCTCCTCCACTATCTACTATGCCGATGCCGTCAAG |
| | | GGCCGCTTCACCATTTCTCGCGACAACGCCCGGAACTCCCTGTACCTCCAG |
| | | ATGAACTCCCTGCGCGCCGAGGACACTGCCGTCTACTACTGCGCGAGGTCG |
| | | TCCGACGGATCCACCTCCTTCGACTACTGGGGGCAAGGAACTCTCGTGACC |
| | | GTGTCCTCCGCTTCTACCAAGGGACCCAGCGTGTTCCCTCTGGCTCCTTCC |
| | | AGCAAGTCTACCTCTGGCGGAACAGCTGCTCTGGGCTGCCTGGTCAAGGAC |
| | | TACTTTCCTGAGCCTGTGACCGTGTCTTGGAACTCTGGCGCTCTGACATCC |
| | | GGCGTGCACACATTTCCAGCTGTGCTGCAGTCCTCCGGCCTGTACTCTCTG |
| | | TCCTCTGTCGTGACCGTGCCTTCCAGCTCTCTGGGAACCCAGACCTACATC |
| | | TGCAATGTGAACCACAAGCCTTCCAACACCAAGGTGGACAAGAGAGTGGAA |
| | | CCCAAGTCCTGCGATAAGACCCACACCTGTCCTCCCTGCCCTGCCCCTGAA |
| | | CTGCTGGGCGGACCTAGCGTGTTCCTGTTCCCTCCAAAGCCCAAGGACACC |
| | | CTGATGATCAGCCGGACCCCTGAAGTGACCTGCGTGGTGGTGGATGTGTCC |
| | | CACGAGGATCCCGAAGTGAAGTTCAATTGGTACGTGGACGGCGTGGAAGTG |
| | | CACAACGCCAAGACCAAGCCCAGAGAGGAACAGTACAACAGCACCTACCGG |
| | | GTGGTGTCCGTGCTGACCGTGCTGCACCAGGACTGGCTGAACGGCAAAGAG |
| | | TACAAGTGCAAGGTGTCCAACAAGGCCCTGCCAGCCCCTATCGAGAAAACC |
| | | ATCAGCAAGGCCAAGGGCCAGCCTAGAGAGCCTCAGGTCTGCACCCTGCCT |
| | | CCCAGCCGGGAAGAGATGACCAAGAACCAGGTGTCCCTGAGCTGCGCCGTG |
| | | AAGGGCTTCTACCCCTCCGATATCGCCGTGGAATGGGAGAGCAACGGCCAG |
| | | CCCGAGAACAACTACAAGACCACCCCTCCCGTGCTGGACAGCGACGGCAGC |
| | | TTCTTCCTGGTGTCCAAACTGACCGTGGACAAGAGCCGGTGGCAGCAGGGC |
| | | AATGTGTTCAGCTGTAGCGTGATGCACGAGGCCCTGCACAACCACTACACC |
| | | CAGAAGTCTCTGAGCCTGAGCCCTGGCAAA |
| SEQ ID | aCSF1R | ATGGAAACCGACACACTGCTGCTGTGGGTGCTGCTCTTGTGGGTGCCAGGA |
| NO: | BI117 | TCTACAGGAGAAGTGCAACTTGTCGAAAGTGGTGGGGGCCTGGTGCAGCCT |
| 289 | A11-R3B | GGGGGCTCCCTGCGGCTGTCCTGCGCCGCCTCCGGATTCACTTTCTCCAAC |
| | VH-hCH1- | GACGAGATGAACTGGATTAGACAGGCTCCTGGAAAGGGTCTCGAATGGGTG |
| | hFc_Hole | TCCTACATTAGCTCATCTTCCAGCACCATCTACTACGCCGACGCAGTGACC |
| | | GGAAGGTTCACCATCTCGAGGGACAACGCCCGCAACTCCCTCTACCTGCAG |
| | | ATGAACTCGCTGCGCGCGGAGGACACCGCTGTGTACTACTGCGCCCGCGGC |
| | | GTGGACGGAGACTTCTCCTTCGACTACTGGGGACAGGGAACTCTTGTGACC |
| | | GTGAGCAGCGCTTCTACCAAGGGACCCAGCGTGTTCCCTCTGGCTCCTTCC |
| | | AGCAAGTCTACCTCTGGCGGAACAGCTGCTCTGGGCTGCCTGGTCAAGGAC |
| | | TACTTTCCTGAGCCTGTGACCGTGTCTTGGAACTCTGGCGCTCTGACATCC |
| | | GGCGTGCACACATTTCCAGCTGTGCTGCAGTCCTCCGGCCTGTACTCTCTG |
| | | TCCTCTGTCGTGACCGTGCCTTCCAGCTCTCTGGGAACCCAGACCTACATC |
| | | TGCAATGTGAACCACAAGCCTTCCAACACCAAGGTGGACAAGAGAGTGGAA |
| | | CCCAAGTCCTGCGATAAGACCCACACCTGTCCTCCCTGCCCTGCCCCTGAA |
| | | CTGCTGGGCGGACCTAGCGTGTTCCTGTTCCCTCCAAAGCCCAAGGACACC |
| | | CTGATGATCAGCCGGACCCCTGAAGTGACCTGCGTGGTGGTGGATGTGTCC |
| | | CACGAGGATCCCGAAGTGAAGTTCAATTGGTACGTGGACGGCGTGGAAGTG |
| | | CACAACGCCAAGACCAAGCCCAGAGAGGAACAGTACAACAGCACCTACCGG |
| | | GTGGTGTCCGTGCTGACCGTGCTGCACCAGGACTGGCTGAACGGCAAAGAG |
| | | TACAAGTGCAAGGTGTCCAACAAGGCCCTGCCAGCCCCTATCGAGAAAACC |
| | | ATCAGCAAGGCCAAGGGCCAGCCTAGAGAGCCTCAGGTCTGCACCCTGCCT |
| | | CCCAGCCGGGAAGAGATGACCAAGAACCAGGTGTCCCTGAGCTGCGCCGTG |
| | | AAGGGCTTCTACCCCTCCGATATCGCCGTGGAATGGGAGAGCAACGGCCAG |
| | | CCCGAGAACAACTACAAGACCACCCCTCCCGTGCTGGACAGCGACGGCAGC |
| | | TTCTTCCTGGTGTCCAAACTGACCGTGGACAAGAGCCGGTGGCAGCAGGGC |
| | | AATGTGTTCAGCTGTAGCGTGATGCACGAGGCCCTGCACAACCACTACACC |
| | | CAGAAGTCTCTGAGCCTGAGCCCTGGCAAA |
| SEQ ID | aCSF1R | ATGGAAACCGACACACTGCTGCTGTGGGTGCTGCTCTTGTGGGTGCCAGGA |
| NO: | BI117 B2- | TCTACAGGAGAAGTGCAACTGGTGGAATCGGGAGGCGGCCTGGTGCAACCT |
| 290 | R3B VH- | GGCGGTAGCCTGCGGCTGAGCTGCGCCGCCTCAGGCTTCACCTTTTCCAAC |
| | hCH1- | GACGAAATGAACTGGATCAGACAGGCGCCTGGCAAAGGACTGGAGTGGGTG |
| | hFc_Hole | TCCTACATCTCGTCCAGGTCAAGCACCATTTATTACGCGGACGCCGTGAAA |
| | | GGGAGATTCACCATCAGCAGAGACAACGCCCGGAACTCCCTCTACCTGCAG |
| | | ATGAACTCCCTGAGGGCCGAGGACACCGCAGTGTACTACTGTGCCAGAAAC |
| | | TCCGACTCCGATGGAAGCTTCGACATCTGGGGACAGGGGACCCTGGTGACT |
| | | GTGTCGTCCGCTTCTACCAAGGGACCCAGCGTGTTCCCTCTGGCTCCTTCC |
| | | AGCAAGTCTACCTCTGGCGGAACAGCTGCTCTGGGCTGCCTGGTCAAGGAC |
| | | TACTTTCCTGAGCCTGTGACCGTGTCTTGGAACTCTGGCGCTCTGACATCC |
| | | GGCGTGCACACATTTCCAGCTGTGCTGCAGTCCTCCGGCCTGTACTCTCTG |
| | | TCCTCTGTCGTGACCGTGCCTTCCAGCTCTCTGGGAACCCAGACCTACATC |
| | | TGCAATGTGAACCACAAGCCTTCCAACACCAAGGTGGACAAGAGAGTGGAA |
| | | CCCAAGTCCTGCGATAAGACCCACACCTGTCCTCCCTGCCCTGCCCCTGAA |
| | | CTGCTGGGCGGACCTAGCGTGTTCCTGTTCCCTCCAAAGCCCAAGGACACC |
| | | CTGATGATCAGCCGGACCCCTGAAGTGACCTGCGTGGTGGTGGATGTGTCC |
| | | CACGAGGATCCCGAAGTGAAGTTCAATTGGTACGTGGACGGCGTGGAAGTG |
| | | CACAACGCCAAGACCAAGCCCAGAGAGGAACAGTACAACAGCACCTACCGG |
| | | GTGGTGTCCGTGCTGACCGTGCTGCACCAGGACTGGCTGAACGGCAAAGAG |
| | | TACAAGTGCAAGGTGTCCAACAAGGCCCTGCCAGCCCCTATCGAGAAAACC |
| | | ATCAGCAAGGCCAAGGGCCAGCCTAGAGAGCCTCAGGTCTGCACCCTGCCT |
| | | CCCAGCCGGGAAGAGATGACCAAGAACCAGGTGTCCCTGAGCTGCGCCGTG |
| | | AAGGGCTTCTACCCCTCCGATATCGCCGTGGAATGGGAGAGCAACGGCCAG |
| | | CCCGAGAACAACTACAAGACCACCCCTCCCGTGCTGGACAGCGACGGCAGC |
| | | TTCTTCCTGGTGTCCAAACTGACCGTGGACAAGAGCCGGTGGCAGCAGGGC |
| | | AATGTGTTCAGCTGTAGCGTGATGCACGAGGCCCTGCACAACCACTACACC |
| | | CAGAAGTCTCTGAGCCTGAGCCCTGGCAAA |
| SEQ ID | aCSF1R | ATGGAAACCGACACACTGCTGCTGTGGGTGCTGCTCTTGTGGGTGCCAGGA |
| NO: | BI117 C5- | TCTACAGGAGAAGTGCAGCTGGTCGAATCGGGGGGAGGACTGGTCCAGCCT |
| 291 | R3B VH- | GGAGGGTCCCTCCGCCTGAGCTGCGCCGCCTCCGGATTCACCTTTTCCAAC |
| | hCH1- | GACGAGATGAACTGGATCAGGCAGGCCCCGGGCAAGGGGCTGGAATGGGTG |
| | hFc_Hole | TCATACATCTCCAGCCGCTCATCGACAATTTACTACGCCGATGCCGTGAAG |
| | | GGTAGATTTACCATCTCCCGGGACAATGCCCGAAACAGCCTGTATCTGCAA |
| | | ATGAACTCCCTGCGGGCGGAGGATACCGCTGTCTACTACTGTGCCAGAGGC |
| | | GTGGATGGGGACTTCAGCTTTGACTTTTGGGGACAGGGCACCCTCGTCACC |
| | | GTGAGCAGCGCTTCTACCAAGGGACCCAGCGTGTTCCCTCTGGCTCCTTCC |
| | | AGCAAGTCTACCTCTGGCGGAACAGCTGCTCTGGGCTGCCTGGTCAAGGAC |
| | | TACTTTCCTGAGCCTGTGACCGTGTCTTGGAACTCTGGCGCTCTGACATCC |
| | | GGCGTGCACACATTTCCAGCTGTGCTGCAGTCCTCCGGCCTGTACTCTCTG |
| | | TCCTCTGTCGTGACCGTGCCTTCCAGCTCTCTGGGAACCCAGACCTACATC |
| | | TGCAATGTGAACCACAAGCCTTCCAACACCAAGGTGGACAAGAGAGTGGAA |
| | | CCCAAGTCCTGCGATAAGACCCACACCTGTCCTCCCTGCCCTGCCCCTGAA |
| | | CTGCTGGGCGGACCTAGCGTGTTCCTGTTCCCTCCAAAGCCCAAGGACACC |
| | | CTGATGATCAGCCGGACCCCTGAAGTGACCTGCGTGGTGGTGGATGTGTCC |
| | | CACGAGGATCCCGAAGTGAAGTTCAATTGGTACGTGGACGGCGTGGAAGTG |
| | | CACAACGCCAAGACCAAGCCCAGAGAGGAACAGTACAACAGCACCTACCGG |
| | | GTGGTGTCCGTGCTGACCGTGCTGCACCAGGACTGGCTGAACGGCAAAGAG |
| | | TACAAGTGCAAGGTGTCCAACAAGGCCCTGCCAGCCCCTATCGAGAAAACC |
| | | ATCAGCAAGGCCAAGGGCCAGCCTAGAGAGCCTCAGGTCTGCACCCTGCCT |
| | | CCCAGCCGGGAAGAGATGACCAAGAACCAGGTGTCCCTGAGCTGCGCCGTG |
| | | AAGGGCTTCTACCCCTCCGATATCGCCGTGGAATGGGAGAGCAACGGCCAG |
| | | CCCGAGAACAACTACAAGACCACCCCTCCCGTGCTGGACAGCGACGGCAGC |
| | | TTCTTCCTGGTGTCCAAACTGACCGTGGACAAGAGCCGGTGGCAGCAGGGC |
| | | AATGTGTTCAGCTGTAGCGTGATGCACGAGGCCCTGCACAACCACTACACC |
| | | CAGAAGTCTCTGAGCCTGAGCCCTGGCAAA |
| SEQ ID | aCSF1R | ATGGAAACCGACACACTGCTGCTGTGGGTGCTGCTCTTGTGGGTGCCAGGA |
| NO: | BI117 C6- | TCTACAGGAGAGGTGCAGCTGGTGGAGTCCGGAGGCGGCCTTGTCCAGCCG |
| 292 | R3B VH- | GGCGGGTCTCTGCGGCTGTCATGTGCGGCCTCGGGATTCACTTTTTCCAAC |
| | hCH1- | GACGAAATGAACTGGATCCGCCAGGCCCCTGGGAAGGGGCTTGAATGGGTG |
| | hFc_Hole | AGCTACATCTCCTCACGGTCCTCCACTATCTATTACGCCGACGCTGTGAAG |
| | | GGCCGCTTCACTATCTCACGCGACAATGCCAGGAACTCTCTCTACCTGCAG |
| | | ATGAACTCCCTGCGGGCCGAAGATACCGCGGTGTACTACTGTGCCCGCTCG |
| | | GGCCGGCTGGACGGGTCATTCGACTACTGGGGACAAGGGACCCTGGTCACC |
| | | GTCTCCTCTGCTTCTACCAAGGGACCCAGCGTGTTCCCTCTGGCTCCTTCC |
| | | AGCAAGTCTACCTCTGGCGGAACAGCTGCTCTGGGCTGCCTGGTCAAGGAC |
| | | TACTTTCCTGAGCCTGTGACCGTGTCTTGGAACTCTGGCGCTCTGACATCC |
| | | GGCGTGCACACATTTCCAGCTGTGCTGCAGTCCTCCGGCCTGTACTCTCTG |
| | | TCCTCTGTCGTGACCGTGCCTTCCAGCTCTCTGGGAACCCAGACCTACATC |
| | | TGCAATGTGAACCACAAGCCTTCCAACACCAAGGTGGACAAGAGAGTGGAA |
| | | CCCAAGTCCTGCGATAAGACCCACACCTGTCCTCCCTGCCCTGCCCCTGAA |
| | | CTGCTGGGCGGACCTAGCGTGTTCCTGTTCCCTCCAAAGCCCAAGGACACC |
| | | CTGATGATCAGCCGGACCCCTGAAGTGACCTGCGTGGTGGTGGATGTGTCC |
| | | CACGAGGATCCCGAAGTGAAGTTCAATTGGTACGTGGACGGCGTGGAAGTG |
| | | CACAACGCCAAGACCAAGCCCAGAGAGGAACAGTACAACAGCACCTACCGG |
| | | GTGGTGTCCGTGCTGACCGTGCTGCACCAGGACTGGCTGAACGGCAAAGAG |
| | | TACAAGTGCAAGGTGTCCAACAAGGCCCTGCCAGCCCCTATCGAGAAAACC |
| | | ATCAGCAAGGCCAAGGGCCAGCCTAGAGAGCCTCAGGTCTGCACCCTGCCT |
| | | CCCAGCCGGGAAGAGATGACCAAGAACCAGGTGTCCCTGAGCTGCGCCGTG |
| | | AAGGGCTTCTACCCCTCCGATATCGCCGTGGAATGGGAGAGCAACGGCCAG |
| | | CCCGAGAACAACTACAAGACCACCCCTCCCGTGCTGGACAGCGACGGCAGC |
| | | TTCTTCCTGGTGTCCAAACTGACCGTGGACAAGAGCCGGTGGCAGCAGGGC |
| | | AATGTGTTCAGCTGTAGCGTGATGCACGAGGCCCTGCACAACCACTACACC |
| | | CAGAAGTCTCTGAGCCTGAGCCCTGGCAAA |
| SEQ ID | aCSF1R | ATGGAAACCGACACACTGCTGCTGTGGGTGCTGCTCTTGTGGGTGCCAGGA |
| NO: | BI117 | TCTACAGGAGAAGTCCAGCTGGTCGAGTCCGGCGGTGGGCTGGTCCAGCCC |
| 293 | D11-R3B | GGCGGATCGCTGCGCCTGTCCTGTGCCGCCTCCGGCTTCACCTTCTCCAAC |
| | VH-hCH1- | GACGAGATGAACTGGATCAGACAGGCCCCCGGTAAGGGACTGGAGTGGGTG |
| | hFc_Hole | AGCTACATCTCCTCGCGCAGCTCCACCATCTACTACGCCGACGCCGTGAAG |
| | | GGAAGGTTCACCATCAGCAGAGATAACGCGCGGAACAGCCTGTACCTGCAG |
| | | ATGAACTCACTGCGGGCTGAAGACACAGCCGTCTATTACTGTGCCAGAGGA |
| | | GTGGAATCCGATTTCAGCTTCGACTACTGGGGACAAGGAACTCTGGTCACG |
| | | GTCAGCTCCGCTTCTACCAAGGGACCCAGCGTGTTCCCTCTGGCTCCTTCC |
| | | AGCAAGTCTACCTCTGGCGGAACAGCTGCTCTGGGCTGCCTGGTCAAGGAC |
| | | TACTTTCCTGAGCCTGTGACCGTGTCTTGGAACTCTGGCGCTCTGACATCC |
| | | GGCGTGCACACATTTCCAGCTGTGCTGCAGTCCTCCGGCCTGTACTCTCTG |
| | | TCCTCTGTCGTGACCGTGCCTTCCAGCTCTCTGGGAACCCAGACCTACATC |
| | | TGCAATGTGAACCACAAGCCTTCCAACACCAAGGTGGACAAGAGAGTGGAA |
| | | CCCAAGTCCTGCGATAAGACCCACACCTGTCCTCCCTGCCCTGCCCCTGAA |
| | | CTGCTGGGCGGACCTAGCGTGTTCCTGTTCCCTCCAAAGCCCAAGGACACC |
| | | CTGATGATCAGCCGGACCCCTGAAGTGACCTGCGTGGTGGTGGATGTGTCC |
| | | CACGAGGATCCCGAAGTGAAGTTCAATTGGTACGTGGACGGCGTGGAAGTG |
| | | CACAACGCCAAGACCAAGCCCAGAGAGGAACAGTACAACAGCACCTACCGG |
| | | GTGGTGTCCGTGCTGACCGTGCTGCACCAGGACTGGCTGAACGGCAAAGAG |
| | | TACAAGTGCAAGGTGTCCAACAAGGCCCTGCCAGCCCCTATCGAGAAAACC |
| | | ATCAGCAAGGCCAAGGGCCAGCCTAGAGAGCCTCAGGTCTGCACCCTGCCT |
| | | CCCAGCCGGGAAGAGATGACCAAGAACCAGGTGTCCCTGAGCTGCGCCGTG |
| | | AAGGGCTTCTACCCCTCCGATATCGCCGTGGAATGGGAGAGCAACGGCCAG |
| | | CCCGAGAACAACTACAAGACCACCCCTCCCGTGCTGGACAGCGACGGCAGC |
| | | TTCTTCCTGGTGTCCAAACTGACCGTGGACAAGAGCCGGTGGCAGCAGGGC |
| | | AATGTGTTCAGCTGTAGCGTGATGCACGAGGCCCTGCACAACCACTACACC |
| | | CAGAAGTCTCTGAGCCTGAGCCCTGGCAAA |
| SEQ ID | aCSF1R | ATGGAAACCGACACACTGCTGCTGTGGGTGCTGCTCTTGTGGGTGCCAGGA |
| NO: | BI123 VH- | TCTACAGGACAGGTGCAGCTTGTGCAAAGCGGGGGAGGCGTGGTGCAGCCG |
| 294 | hCH1- | GGACGGAGCCTGCGGCTGTCGTGCGCCGCGAGCGGTTTCACCTTCTCCTCC |
| | hFc_Hole | TACGCTATGCACTGGGTCCGCCAGGCGCCGGGCAAGGGCCTCGAATGGGTG |
| | | GCCGTGATCAGCTACGATGGCTCCAACAAGTACTACGCCGACTCAGTGAAG |
| | | GGAAGGTTCACCATCTCAAGAGATAACAGCAAGAACACGCTGTACCTCCAG |
| | | ATGAACTCCCTCCGGGCCGAAGACACAGCCGTCTACTACTGTGCGAGAGAG |
| | | CTCGATTACGGCGATTACGGCCTGGACGTGTGGGGCAAGGGAACCACGGTG |
| | | ACCGTCTCCTCCGCTTCTACCAAGGGACCCAGCGTGTTCCCTCTGGCTCCT |
| | | TCCAGCAAGTCTACCTCTGGCGGAACAGCTGCTCTGGGCTGCCTGGTCAAG |
| | | GACTACTTTCCTGAGCCTGTGACCGTGTCTTGGAACTCTGGCGCTCTGACA |
| | | TCCGGCGTGCACACATTTCCAGCTGTGCTGCAGTCCTCCGGCCTGTACTCT |
| | | CTGTCCTCTGTCGTGACCGTGCCTTCCAGCTCTCTGGGAACCCAGACCTAC |
| | | ATCTGCAATGTGAACCACAAGCCTTCCAACACCAAGGTGGACAAGAGAGTG |
| | | GAACCCAAGTCCTGCGATAAGACCCACACCTGTCCTCCCTGCCCTGCCCCT |
| | | GAACTGCTGGGCGGACCTAGCGTGTTCCTGTTCCCTCCAAAGCCCAAGGAC |
| | | ACCCTGATGATCAGCCGGACCCCTGAAGTGACCTGCGTGGTGGTGGATGTG |
| | | TCCCACGAGGATCCCGAAGTGAAGTTCAATTGGTACGTGGACGGCGTGGAA |
| | | GTGCACAACGCCAAGACCAAGCCCAGAGAGGAACAGTACAACAGCACCTAC |
| | | CGGGTGGTGTCCGTGCTGACCGTGCTGCACCAGGACTGGCTGAACGGCAAA |
| | | GAGTACAAGTGCAAGGTGTCCAACAAGGCCCTGCCAGCCCCTATCGAGAAA |
| | | ACCATCAGCAAGGCCAAGGGCCAGCCTAGAGAGCCTCAGGTCTGCACCCTG |
| | | CCTCCCAGCCGGGAAGAGATGACCAAGAACCAGGTGTCCCTGAGCTGCGCC |
| | | GTGAAGGGCTTCTACCCCTCCGATATCGCCGTGGAATGGGAGAGCAACGGC |
| | | CAGCCCGAGAACAACTACAAGACCACCCCTCCCGTGCTGGACAGCGACGGC |
| | | AGCTTCTTCCTGGTGTCCAAACTGACCGTGGACAAGAGCCGGTGGCAGCAG |
| | | GGCAATGTGTTCAGCTGTAGCGTGATGCACGAGGCCCTGCACAACCACTAC |
| | | ACCCAGAAGTCTCTGAGCCTGAGCCCTGGCAAA |
| SEQ ID | aCSF1R | ATGGAAACCGACACACTGCTGCTGTGGGTGCTGCTCTTGTGGGTGCCAGGA |
| NO: | BI123 A5- | TCTACAGGACAAGTGCAGCTCGTGCAGTCAGGCGGTGGCGTGGTGCAGCCC |
| 295 | R3B VH- | GGCCGGTCTCTGAGGCTCAGCTGCGCCGCCTCGGGCTTCACCTTCTCGTCA |
| | hCH1- | TACGCCATGCACTGGGTGCGCCAGGCCCCCGGGAAGGGACTGGAATGGGTC |
| | hFc_Hole | GCCGTGATCTCCTACGATGGATCGAACAAGTACTACGCCGACTCCGTGAAG |
| | | GGGCGGTTCACCATCTCCCGCGACAACTCCAAGAACACCCTTTATCTGCAA |
| | | ATGAACTCGCTCAGAGCAGAGGACACCGCTGTGTACTACTGCGCCAGAGAG |
| | | CTGGACTACGGTGATTGGGGACTGGACGTGTGGGGCAAGGGTACCACCGTG |
| | | ACTGTGTCGTCCGCTTCTACCAAGGGACCCAGCGTGTTCCCTCTGGCTCCT |
| | | TCCAGCAAGTCTACCTCTGGCGGAACAGCTGCTCTGGGCTGCCTGGTCAAG |
| | | GACTACTTTCCTGAGCCTGTGACCGTGTCTTGGAACTCTGGCGCTCTGACA |
| | | TCCGGCGTGCACACATTTCCAGCTGTGCTGCAGTCCTCCGGCCTGTACTCT |
| | | CTGTCCTCTGTCGTGACCGTGCCTTCCAGCTCTCTGGGAACCCAGACCTAC |
| | | ATCTGCAATGTGAACCACAAGCCTTCCAACACCAAGGTGGACAAGAGAGTG |
| | | GAACCCAAGTCCTGCGATAAGACCCACACCTGTCCTCCCTGCCCTGCCCCT |
| | | GAACTGCTGGGCGGACCTAGCGTGTTCCTGTTCCCTCCAAAGCCCAAGGAC |
| | | ACCCTGATGATCAGCCGGACCCCTGAAGTGACCTGCGTGGTGGTGGATGTG |
| | | TCCCACGAGGATCCCGAAGTGAAGTTCAATTGGTACGTGGACGGCGTGGAA |
| | | GTGCACAACGCCAAGACCAAGCCCAGAGAGGAACAGTACAACAGCACCTAC |
| | | CGGGTGGTGTCCGTGCTGACCGTGCTGCACCAGGACTGGCTGAACGGCAAA |
| | | GAGTACAAGTGCAAGGTGTCCAACAAGGCCCTGCCAGCCCCTATCGAGAAA |
| | | ACCATCAGCAAGGCCAAGGGCCAGCCTAGAGAGCCTCAGGTCTGCACCCTG |
| | | CCTCCCAGCCGGGAAGAGATGACCAAGAACCAGGTGTCCCTGAGCTGCGCC |
| | | GTGAAGGGCTTCTACCCCTCCGATATCGCCGTGGAATGGGAGAGCAACGGC |
| | | CAGCCCGAGAACAACTACAAGACCACCCCTCCCGTGCTGGACAGCGACGGC |
| | | AGCTTCTTCCTGGTGTCCAAACTGACCGTGGACAAGAGCCGGTGGCAGCAG |
| | | GGCAATGTGTTCAGCTGTAGCGTGATGCACGAGGCCCTGCACAACCACTAC |
| | | ACCCAGAAGTCTCTGAGCCTGAGCCCTGGCAAA |
| SEQ ID | aCSF1R | ATGGAAACCGACACACTGCTGCTGTGGGTGCTGCTCTTGTGGGTGCCAGGA |
| NO: | BI123 B6- | TCTACAGGACAAGTGCAACTGGTGCAATCCGGAGGGGGTGTGGTCCAACCG |
| 296 | R3B VH- | GGACGCAGCCTGCGGCTGTCCTGCGCCGCCAGCGGCTTCACATTCTCCTCC |
| | hCH1- | TACGCCATGCACTGGGTCCGCCAGGCGCCAGGTAAGGGACTCGAATGGGTG |
| | hFc_Hole | GCAGTGATCCCTTACTACGGATCAAACAAGTATTACGCCGATTCCGTGAAG |
| | | GGCCGCTTCACCATCAGCAGAGACAACTCCAAGAACACCCTCTACCTGCAG |
| | | ATGAACTCCCTTCGCGCCGAGGATACCGCCGTCTACTACTGCGCCCGCGAG |
| | | CTGGACTACGGTGACTACGGACTCGATGTGTGGGGAAAGGGTACCACTGTG |
| | | ACCGTGAGCTCCGCTTCTACCAAGGGACCCAGCGTGTTCCCTCTGGCTCCT |
| | | TCCAGCAAGTCTACCTCTGGCGGAACAGCTGCTCTGGGCTGCCTGGTCAAG |
| | | GACTACTTTCCTGAGCCTGTGACCGTGTCTTGGAACTCTGGCGCTCTGACA |
| | | TCCGGCGTGCACACATTTCCAGCTGTGCTGCAGTCCTCCGGCCTGTACTCT |
| | | CTGTCCTCTGTCGTGACCGTGCCTTCCAGCTCTCTGGGAACCCAGACCTAC |
| | | ATCTGCAATGTGAACCACAAGCCTTCCAACACCAAGGTGGACAAGAGAGTG |
| | | GAACCCAAGTCCTGCGATAAGACCCACACCTGTCCTCCCTGCCCTGCCCCT |
| | | GAACTGCTGGGCGGACCTAGCGTGTTCCTGTTCCCTCCAAAGCCCAAGGAC |
| | | ACCCTGATGATCAGCCGGACCCCTGAAGTGACCTGCGTGGTGGTGGATGTG |
| | | TCCCACGAGGATCCCGAAGTGAAGTTCAATTGGTACGTGGACGGCGTGGAA |
| | | GTGCACAACGCCAAGACCAAGCCCAGAGAGGAACAGTACAACAGCACCTAC |
| | | CGGGTGGTGTCCGTGCTGACCGTGCTGCACCAGGACTGGCTGAACGGCAAA |
| | | GAGTACAAGTGCAAGGTGTCCAACAAGGCCCTGCCAGCCCCTATCGAGAAA |
| | | ACCATCAGCAAGGCCAAGGGCCAGCCTAGAGAGCCTCAGGTCTGCACCCTG |
| | | CCTCCCAGCCGGGAAGAGATGACCAAGAACCAGGTGTCCCTGAGCTGCGCC |
| | | GTGAAGGGCTTCTACCCCTCCGATATCGCCGTGGAATGGGAGAGCAACGGC |
| | | CAGCCCGAGAACAACTACAAGACCACCCCTCCCGTGCTGGACAGCGACGGC |
| | | AGCTTCTTCCTGGTGTCCAAACTGACCGTGGACAAGAGCCGGTGGCAGCAG |
| | | GGCAATGTGTTCAGCTGTAGCGTGATGCACGAGGCCCTGCACAACCACTAC |
| | | ACCCAGAAGTCTCTGAGCCTGAGCCCTGGCAAA |
| SEQ ID | aCSF1R | ATGGAAACCGACACACTGCTGCTGTGGGTGCTGCTCTTGTGGGTGCCAGGA |
| NO: | BI123 C1- | TCTACAGGACAGGTGCAACTGGTCCAGTCGGGCGGAGGAGTGGTCCAGCCG |
| 297 | R3B VH- | GGTAGGTCACTGCGGCTGTCATGTGCCGCTAGCGGATTCACTTTCTCTTCC |
| | hCH1- | TACGCAATGCACTGGGTGAGACAAGCCCCTGGCAAGGGACTGGAGTGGGTC |
| | hFc_Hole | GCCGTGATCTCGTATGACGGGTCCGATAAGTACTACGCTGATTCCGTGAAG |
| | | GGACGCTTCACGATCAGCCGCGACAACTCCAAAAACACTCTCTACCTGCAA |
| | | ATGAATTCCCTTCGCGCCGAAGATACTGCAGTGTACTACTGCGCCAGAGAG |
| | | CTGGACTACGGAGACTACGGCCTGGACGTGTGGGGCAAGGGCACCACTGTG |
| | | ACCGTCTCCTCCGCTTCTACCAAGGGACCCAGCGTGTTCCCTCTGGCTCCT |
| | | TCCAGCAAGTCTACCTCTGGCGGAACAGCTGCTCTGGGCTGCCTGGTCAAG |
| | | GACTACTTTCCTGAGCCTGTGACCGTGTCTTGGAACTCTGGCGCTCTGACA |
| | | TCCGGCGTGCACACATTTCCAGCTGTGCTGCAGTCCTCCGGCCTGTACTCT |
| | | CTGTCCTCTGTCGTGACCGTGCCTTCCAGCTCTCTGGGAACCCAGACCTAC |
| | | ATCTGCAATGTGAACCACAAGCCTTCCAACACCAAGGTGGACAAGAGAGTG |
| | | GAACCCAAGTCCTGCGATAAGACCCACACCTGTCCTCCCTGCCCTGCCCCT |
| | | GAACTGCTGGGCGGACCTAGCGTGTTCCTGTTCCCTCCAAAGCCCAAGGAC |
| | | ACCCTGATGATCAGCCGGACCCCTGAAGTGACCTGCGTGGTGGTGGATGTG |
| | | TCCCACGAGGATCCCGAAGTGAAGTTCAATTGGTACGTGGACGGCGTGGAA |
| | | GTGCACAACGCCAAGACCAAGCCCAGAGAGGAACAGTACAACAGCACCTAC |
| | | CGGGTGGTGTCCGTGCTGACCGTGCTGCACCAGGACTGGCTGAACGGCAAA |
| | | GAGTACAAGTGCAAGGTGTCCAACAAGGCCCTGCCAGCCCCTATCGAGAAA |
| | | ACCATCAGCAAGGCCAAGGGCCAGCCTAGAGAGCCTCAGGTCTGCACCCTG |
| | | CCTCCCAGCCGGGAAGAGATGACCAAGAACCAGGTGTCCCTGAGCTGCGCC |
| | | GTGAAGGGCTTCTACCCCTCCGATATCGCCGTGGAATGGGAGAGCAACGGC |
| | | CAGCCCGAGAACAACTACAAGACCACCCCTCCCGTGCTGGACAGCGACGGC |
| | | AGCTTCTTCCTGGTGTCCAAACTGACCGTGGACAAGAGCCGGTGGCAGCAG |
| | | GGCAATGTGTTCAGCTGTAGCGTGATGCACGAGGCCCTGCACAACCACTAC |
| | | ACCCAGAAGTCTCTGAGCCTGAGCCCTGGCAAA |
| SEQ ID | aCSF1R | ATGGAAACCGACACACTGCTGCTGTGGGTGCTGCTCTTGTGGGTGCCAGGA |
| NO: | BI117 VL- | TCTACAGGACAACTGGTGCTGACCCAGCCCCCTTCCGTCTCCGGCGCCCCG |
| 298 | hCLIg_v1 | GGCCAGCGCGTCACTATCTCCTGTACCGGCAGCTCCAGCAACATCGGAGCA |
| | | GGATACGACGTGCACTGGTACCAACAGCTCCCCGGAACCGCGCCTAAGCTG |
| | | CTGATCTACGGGAACTCTAACCGGCCGTCCGGCGTGCCTGACCGCTTCAGC |
| | | GGATCGAAGTCCGGCACCAGCGCCTCCCTGGCCATCACTGGACTGCAGGCC |
| | | GAGGACGAAGCCGATTACTACTGCCAGTCCTACGACTCCTCCCTGTCCGAG |
| | | GAAGTGTTCGGAGGCGGGACGAAGCTCACCGTCCTGGGACAACCTAAGGCC |
| | | AATCCTACCGTGACACTGTTCCCTCCATCCTCCGAGGAACTGCAGGCCAAC |
| | | AAGGCTACCCTCGTGTGCCTGATCTCCGACTTTTACCCTGGCGCTGTGACC |
| | | GTGGCCTGGAAGGCTGATGGATCTCCTGTGAAGGCTGGCGTGGAAACCACC |
| | | AAGCCTTCCAAGCAGTCCAACAACAAATACGCCGCCTCCTCCTACCTGTCT |
| | | CTGACCCCTGAACAGTGGAAGTCCCACCGGTCCTACAGCTGCCAAGTGACC |
| | | CATGAGGGCTCCACCGTGGAAAAGACCGTGGCTCCTACCGAGTGCTCC |
| SEQ ID | aCSF1R | ATGGAAACCGACACACTGCTGCTGTGGGTGCTGCTCTTGTGGGTGCCAGGA |
| NO: | BI123 VL- | TCTACAGGATCCCTCGTGCTGACCCAACCTCCTTCAGTCTCCGTGGCCCCC |
| 299 | hCLIg_v1 | GGCAAGACCGCCCGCATCACCTGTGGCGGTAACAACATCGGCTCCAAGTCA |
| | | GTCCACTGGTACCAGCAGAAGCCGGGCCAAGCTCCCGTGCTGGTCGTGTAC |
| | | GACGACTCAGACCGCCCCAGCGGTATCCCTGAGAGATTTAGCGGATCCAAC |
| | | TCCGGGAACACCGCTACCCTGACCATCTCAAGGGTGGAGGCCGGAGACGAA |
| | | GCCGACTACTACTGCCAGGTGTGGGATTCCAGCTCCGACCACGTGGTGTTC |
| | | GGCGGCGGCACTCAGCTCACCGTGCTGGGACAACCTAAGGCCAATCCTACC |
| | | GTGACACTGTTCCCTCCATCCTCCGAGGAACTGCAGGCCAACAAGGCTACC |
| | | CTCGTGTGCCTGATCTCCGACTTTTACCCTGGCGCTGTGACCGTGGCCTGG |
| | | AAGGCTGATGGATCTCCTGTGAAGGCTGGCGTGGAAACCACCAAGCCTTCC |
| | | AAGCAGTCCAACAACAAATACGCCGCCTCCTCCTACCTGTCTCTGACCCCT |
| | | GAACAGTGGAAGTCCCACCGGTCCTACAGCTGCCAAGTGACCCATGAGGGC |
| | | TCCACCGTGGAAAAGACCGTGGCTCCTACCGAGTGCTCC |
| SEQ ID | aCCR2 | ATGGAAACCGACACACTGCTGCTGTGGGTGCTGCTCTTGTGGGTGCCAGGA |
| NO: | Hum1 VH- | TCTACAGGAGAAGTGCAGCTGGTGGAAAGCGGTGGCGGTCTGGTGCAGCCT |
| 300 | hCH1- | GGCGGTTCCCTCCGGCTGTCATGTGCTGCCTCCGGATTCTCGTTCAACGCG |
| | hFc_Knob | TATGCCATGAACTGGGTGAGGCAGGCCCCGGGAAAGGGACTGGAATGGGTG |
| | | GGGAGAATCCGGACCAAGAACAATAACTACGCTACTTACTACGCCGACTCC |
| | | GTCAAGGACCGGTTCACAATTTCCCGGGACGACAGCAAGAGCTCACTGTAC |
| | | CTTCAGATGAACAGCCTCAAAACCGAGGACACCGCCGTGTACTACTGTGCC |
| | | ACCTTTTACGGCAACGGTGTGTGGGGACAGGGAACAACTGTCACCGTGTCC |
| | | TCCGCTTCTACCAAGGGACCCAGCGTGTTCCCTCTGGCTCCTTCCAGCAAG |
| | | TCTACCTCTGGCGGAACAGCTGCTCTGGGCTGCCTGGTCAAGGACTACTTT |
| | | CCTGAGCCTGTGACCGTGTCTTGGAACTCTGGCGCTCTGACATCCGGCGTG |
| | | CACACATTTCCAGCTGTGCTGCAGTCCTCCGGCCTGTACTCTCTGTCCTCT |
| | | GTCGTGACCGTGCCTTCCAGCTCTCTGGGAACCCAGACCTACATCTGCAAT |
| | | GTGAACCACAAGCCTTCCAACACCAAGGTGGACAAGAGAGTGGAACCCAAG |
| | | TCCTGCGATAAGACCCACACATGTCCTCCATGCCCTGCCCCTGAGCTGCTG |
| | | GGCGGACCTTCCGTGTTCCTGTTCCCTCCAAAGCCCAAGGACACCCTGATG |
| | | ATCAGCCGGACCCCTGAAGTGACCTGCGTGGTGGTGGATGTGTCCCACGAG |
| | | GATCCCGAAGTGAAGTTCAATTGGTACGTGGACGGCGTGGAAGTGCACAAC |
| | | GCCAAGACCAAGCCCAGAGAGGAACAGTACAACAGCACCTACCGGGTGGTG |
| | | TCCGTGCTGACCGTGCTGCATCAGGACTGGCTGAACGGCAAAGAGTACAAG |
| | | TGCAAGGTGTCCAACAAGGCCCTGCCTGCCCCTATCGAGAAAACCATCAGC |
| | | AAGGCCAAGGGCCAGCCCCGCGAACCTCAGGTGTACACACTGCCTCCCTGC |
| | | CGGGAAGAGATGACCAAGAACCAGGTGTCCCTGTGGTGCCTGGTCAAGGGC |
| | | TTCTACCCCTCCGATATCGCCGTGGAATGGGAGAGCAACGGCCAGCCCGAG |
| | | AACAACTACAAGACCACCCCTCCCGTGCTGGACAGCGACGGCAGCTTCTTC |
| | | CTGTACTCCAAACTGACCGTGGACAAGAGCCGGTGGCAGCAGGGCAATGTG |
| | | TTCAGCTGTAGCGTGATGCACGAGGCCCTGCACAACCACTACACCCAGAAG |
| | | TCCCTGAGCCTGTCTCCTGGCAAA |
| SEQ ID | aCCR2 | ATGGAAACCGACACACTGCTGCTGTGGGTGCTGCTCTTGTGGGTGCCAGGA |
| NO: | Hum2 VH- | TCTACAGGAGAGGTGCAGCTGGTTGAATCTGGCGGAGGATTGGTTCAGCCT |
| 301 | hCH1- | GGCGGCTCTCTGAGACTGTCTTGTGCCGCCTCTGGCTTCTCCTTCAACGCC |
| | hFc_Knob | TACGCCATGAACTGGGTCCGACAGGCTCCTGGCAAAGGCCTGGAATGGGTC |
| | | GGAAGAATCCGGACCAAGAACAACAACTACGCCACCTACTACGCCGACTCC |
| | | GTGAAGGACCGGTTCACCATCTCTCGGGACGACTCCAAGTCCAGCCTGTAC |
| | | CTGCAGATGAACTCCCTGAAAACCGAGGACACCGCCGTGTACTACTGCGCC |
| | | ACCTTTTACGGCAATGGCGTGTGGGGCCAGGGCACCACAGTTACAGTTTCT |
| | | TCCGCTTCTACCAAGGGACCCAGCGTGTTCCCTCTGGCTCCTTCCAGCAAG |
| | | TCTACCTCTGGCGGAACAGCTGCTCTGGGCTGCCTGGTCAAGGACTACTTT |
| | | CCTGAGCCTGTGACCGTGTCTTGGAACTCTGGCGCTCTGACATCCGGCGTG |
| | | CACACATTTCCAGCTGTGCTGCAGTCCTCCGGCCTGTACTCTCTGTCCTCT |
| | | GTCGTGACCGTGCCTTCCAGCTCTCTGGGAACCCAGACCTACATCTGCAAT |
| | | GTGAACCACAAGCCTTCCAACACCAAGGTGGACAAGAGAGTGGAACCCAAG |
| | | TCCTGCGATAAGACCCACACATGTCCTCCATGCCCTGCCCCTGAGCTGCTG |
| | | GGCGGACCTTCCGTGTTCCTGTTCCCTCCAAAGCCCAAGGACACCCTGATG |
| | | ATCAGCCGGACCCCTGAAGTGACCTGCGTGGTGGTGGATGTGTCCCACGAG |
| | | GATCCCGAAGTGAAGTTCAATTGGTACGTGGACGGCGTGGAAGTGCACAAC |
| | | GCCAAGACCAAGCCCAGAGAGGAACAGTACAACAGCACCTACCGGGTGGTG |
| | | TCCGTGCTGACCGTGCTGCATCAGGACTGGCTGAACGGCAAAGAGTACAAG |
| | | TGCAAGGTGTCCAACAAGGCCCTGCCTGCCCCTATCGAGAAAACCATCAGC |
| | | AAGGCCAAGGGCCAGCCCCGCGAACCTCAGGTGTACACACTGCCTCCCTGC |
| | | CGGGAAGAGATGACCAAGAACCAGGTGTCCCTGTGGTGCCTGGTCAAGGGC |
| | | TTCTACCCCTCCGATATCGCCGTGGAATGGGAGAGCAACGGCCAGCCCGAG |
| | | AACAACTACAAGACCACCCCTCCCGTGCTGGACAGCGACGGCAGCTTCTTC |
| | | CTGTACTCCAAACTGACCGTGGACAAGAGCCGGTGGCAGCAGGGCAATGTG |
| | | TTCAGCTGTAGCGTGATGCACGAGGCCCTGCACAACCACTACACCCAGAAG |
| | | TCCCTGAGCCTGTCTCCTGGCAAA |
| SEQ ID | aCCR2 | ATGGAAACCGACACACTGCTGCTGTGGGTGCTGCTCTTGTGGGTGCCAGGA |
| NO: | VL- | TCTACAGGAGACGTCGTCATGACCCAATCCCCGCTCTCCCTGCCGGTGACT |
| 302 | hCLIg_vk | CTCGGCCAGCCGGCGTCCATCTCGTGCAAGTCCTCCCAATCCCTGCTGGAT |
| | | TCGGATGGGAAGACTTTCCTGAACTGGCTGCAGCAACGGCCCGGCCAGTCG |
| | | CCACGGCGGCTGATCTACCTGGTGTCCAAGCTGGATTCAGGTGTGCCTGAT |
| | | AGATTCTCCGGCAGCGGATCGGGGACCGACTTCACGCTGAAGATCTCCAGA |
| | | GTGGAAGCAGAGGACGTGGGCGTCTATTACTGTTGGCAGGGGACTCATTTC |
| | | CCCTATACCTTTGGAGGTGGTACTAAGGTGGAAATTAAGAGAACAGTGGCC |
| | | GCTCCTTCCGTGTTCATCTTCCCACCCTCCGACGAGCAGCTGAAGTCCGGC |
| | | ACCGCTTCTGTCGTGTGCCTGCTCAACAACTTCTACCCTCGGGAAGCCAAG |
| | | GTGCAGTGGAAGGTGGACAACGCCCTGCAGTCCGGCAACTCCCAGGAATCC |
| | | GTCACCGAGCAGGACTCCAAGGACAGCACCTACTCCCTGTCCTCCACCCTG |
| | | ACCCTGTCCAAGGCCGACTACGAGAAGCACAAGGTGTACGCCTGCGAAGTG |
| | | ACCCACCAGGGCCTGAGCAGCCCCGTGACCAAGTCCTTCAACCGGGGCGAG |
| | | TGC |
| SEQ ID | aCSF1R | ATGGAAACCGACACACTGCTGCTGTGGGTGCTGCTCTTGTGGGTGCCAGGA |
| NO: | BI117 VH- | TCTACAGGAGAAGTGCAGCTGGTCGAAAGCGGAGGGGGTCTGGTCCAGCCT |
| 303 | hCH1- | GGAGGGTCCCTGCGGCTGTCCTGCGCCGCTTCCGGCTTCACCTTCTCCAAC |
| | hFc_Hole- | GATGAAATGAACTGGATCCGCCAAGCCCCTGGAAAGGGCCTGGAATGGGTG |
| | 3 x 4GS- | TCGTACATCTCGAGCAGGTCCAGCACTATCTACTATGCCGACGCGGTGAAG |
| | BH794_VH- | GGACGCTTCACTATCAGCCGCGATAACGCACGCAATTCCCTCTACCTGCAG |
| | 3 x 4GS- | ATGAACTCGCTCAGAGCTGAGGATACTGCCGTCTACTACTGCGCCCGCGGC |
| | BH794_VL | GTGGACGGCGATTTTTCCTTCGATTACTGGGGACAGGGCACTCTTGTCACC |
| | | GTGTCAAGCGCTTCTACCAAGGGACCCAGCGTGTTCCCTCTGGCTCCTTCC |
| | | AGCAAGTCTACCTCTGGCGGAACAGCTGCTCTGGGCTGCCTGGTCAAGGAC |
| | | TACTTTCCTGAGCCTGTGACCGTGTCTTGGAACTCTGGCGCTCTGACATCC |
| | | GGCGTGCACACATTTCCAGCTGTGCTGCAGTCCTCCGGCCTGTACTCTCTG |
| | | TCCTCTGTCGTGACCGTGCCTTCCAGCTCTCTGGGAACCCAGACCTACATC |
| | | TGCAATGTGAACCACAAGCCTTCCAACACCAAGGTGGACAAGAGAGTGGAA |
| | | CCCAAGTCCTGCGATAAGACCCACACCTGTCCTCCCTGCCCTGCCCCTGAA |
| | | CTGCTGGGCGGACCTAGCGTGTTCCTGTTCCCTCCAAAGCCCAAGGACACC |
| | | CTGATGATCAGCCGGACCCCTGAAGTGACCTGCGTGGTGGTGGATGTGTCC |
| | | CACGAGGATCCCGAAGTGAAGTTCAATTGGTACGTGGACGGCGTGGAAGTG |
| | | CACAACGCCAAGACCAAGCCCAGAGAGGAACAGTACAACAGCACCTACCGG |
| | | GTGGTGTCCGTGCTGACCGTGCTGCACCAGGACTGGCTGAACGGCAAAGAG |
| | | TACAAGTGCAAGGTGTCCAACAAGGCCCTGCCAGCCCCTATCGAGAAAACC |
| | | ATCAGCAAGGCCAAGGGCCAGCCTAGAGAGCCTCAGGTCTGCACCCTGCCT |
| | | CCCAGCCGGGAAGAGATGACCAAGAACCAGGTGTCCCTGAGCTGCGCCGTG |
| | | AAGGGCTTCTACCCCTCCGATATCGCCGTGGAATGGGAGAGCAACGGCCAG |
| | | CCCGAGAACAACTACAAGACCACCCCTCCCGTGCTGGACAGCGACGGCAGC |
| | | TTCTTCCTGGTGTCCAAACTGACCGTGGACAAGAGCCGGTGGCAGCAGGGC |
| | | AATGTGTTCAGCTGTAGCGTGATGCACGAGGCCCTGCACAACCACTACACC |
| | | CAGAAGTCTCTGAGCCTGAGCCCTGGCAAAGGTGGCGGAGGAAGCGGAGGC |
| | | GGAGGTTCTGGCGGCGGAGGATCTCAGGTGCAGCTGGTTCAGTCTGGCGCC |
| | | GAAGTGAAGAAACCTGGCTCCTCCGTGAAGGTGTCCTGCAAGGCTTCTGGC |
| | | GGCACCTTCTCCTCTTACGCCATCTCCTGGGTCCGACAGGCTCCTGGACAA |
| | | GGCTTGGAATGGATGGGCGGCATCATCCCCATCTTCGGCACCGCCAATTAC |
| | | GCCCAGAAATTCCAGGGCAGAGTGACCATCACCGCCGACAAGTCTACCTCC |
| | | ACCGCCTACATGGAACTGTCCAGCCTGAGATCTGAGGACACCGCCGTGTAC |
| | | TACTGTGCCAGAGGCGGCTACTCTTACGGCTCCCTGGATTATTGGGGCCAG |
| | | GGCACACTGGTCACCGTGTCCTCTGGTGGCGGAGGAAGCGGAGGCGGAGGT |
| | | TCTGGCGGCGGAGGATCTGACATCCAGATGACCCAGTCTCCATCCTCTCTG |
| | | TCCGCCTCTGTGGGCGACAGAGTGACCATCACCTGTCGGGCCTCTCAGTCC |
| | | ATCATCTCCGACCTGAACTGGTATCAGCAGAAGCCCGGCAAGGCCCCTAAG |
| | | CTGCTGATCCATGCTGCTTCCAGTCTGCAGTCTGGCGTGCCCTCTAGATTC |
| | | TCCGGCTCTGGCTCTGGCACCGACTTTACCCTGACAATCTCCAGCCTGCAG |
| | | CCTGAGGACTTCGCCACCTACTACTGCCAGCAGTCCTACAGCACCCCTTAC |
| | | ACCTTTGGCCAGGGCACCAAGCTGGAAATCAAG |
| SEQ ID | aCSF1R | ATGGAAACCGACACACTGCTGCTGTGGGTGCTGCTCTTGTGGGTGCCAGGA |
| NO: | BI123 VH- | TCTACAGGACAGGTGCAGCTTGTGCAAAGCGGGGGAGGCGTGGTGCAGCCG |
| 304 | hCH1- | GGACGGAGCCTGCGGCTGTCGTGCGCCGCGAGCGGTTTCACCTTCTCCTCC |
| | hFc_Hole- | TACGCTATGCACTGGGTCCGCCAGGCGCCGGGCAAGGGCCTCGAATGGGTG |
| | 3 x 4GS- | GCCGTGATCAGCTACGATGGCTCCAACAAGTACTACGCCGACTCAGTGAAG |
| | BH794_VH- | GGAAGGTTCACCATCTCAAGAGATAACAGCAAGAACACGCTGTACCTCCAG |
| | 3 x 4GS- | ATGAACTCCCTCCGGGCCGAAGACACAGCCGTCTACTACTGTGCGAGAGAG |
| | BH794_VL | CTCGATTACGGCGATTACGGCCTGGACGTGTGGGGCAAGGGAACCACGGTG |
| | | ACCGTCTCCTCCGCTTCTACCAAGGGACCCAGCGTGTTCCCTCTGGCTCCT |
| | | TCCAGCAAGTCTACCTCTGGCGGAACAGCTGCTCTGGGCTGCCTGGTCAAG |
| | | GACTACTTTCCTGAGCCTGTGACCGTGTCTTGGAACTCTGGCGCTCTGACA |
| | | TCCGGCGTGCACACATTTCCAGCTGTGCTGCAGTCCTCCGGCCTGTACTCT |
| | | CTGTCCTCTGTCGTGACCGTGCCTTCCAGCTCTCTGGGAACCCAGACCTAC |
| | | ATCTGCAATGTGAACCACAAGCCTTCCAACACCAAGGTGGACAAGAGAGTG |
| | | GAACCCAAGTCCTGCGATAAGACCCACACCTGTCCTCCCTGCCCTGCCCCT |
| | | GAACTGCTGGGCGGACCTAGCGTGTTCCTGTTCCCTCCAAAGCCCAAGGAC |
| | | ACCCTGATGATCAGCCGGACCCCTGAAGTGACCTGCGTGGTGGTGGATGTG |
| | | TCCCACGAGGATCCCGAAGTGAAGTTCAATTGGTACGTGGACGGCGTGGAA |
| | | GTGCACAACGCCAAGACCAAGCCCAGAGAGGAACAGTACAACAGCACCTAC |
| | | CGGGTGGTGTCCGTGCTGACCGTGCTGCACCAGGACTGGCTGAACGGCAAA |
| | | GAGTACAAGTGCAAGGTGTCCAACAAGGCCCTGCCAGCCCCTATCGAGAAA |
| | | ACCATCAGCAAGGCCAAGGGCCAGCCTAGAGAGCCTCAGGTCTGCACCCTG |
| | | CCTCCCAGCCGGGAAGAGATGACCAAGAACCAGGTGTCCCTGAGCTGCGCC |
| | | GTGAAGGGCTTCTACCCCTCCGATATCGCCGTGGAATGGGAGAGCAACGGC |
| | | CAGCCCGAGAACAACTACAAGACCACCCCTCCCGTGCTGGACAGCGACGGC |
| | | AGCTTCTTCCTGGTGTCCAAACTGACCGTGGACAAGAGCCGGTGGCAGCAG |
| | | GGCAATGTGTTCAGCTGTAGCGTGATGCACGAGGCCCTGCACAACCACTAC |
| | | ACCCAGAAGTCTCTGAGCCTGAGCCCTGGCAAAGGTGGCGGAGGAAGCGGA |
| | | GGCGGAGGTTCTGGCGGCGGAGGATCTCAGGTGCAGCTGGTTCAGTCTGGC |
| | | GCCGAAGTGAAGAAACCTGGCTCCTCCGTGAAGGTGTCCTGCAAGGCTTCT |
| | | GGCGGCACCTTCTCCTCTTACGCCATCTCCTGGGTCCGACAGGCTCCTGGA |
| | | CAAGGCTTGGAATGGATGGGCGGCATCATCCCCATCTTCGGCACCGCCAAT |
| | | TACGCCCAGAAATTCCAGGGCAGAGTGACCATCACCGCCGACAAGTCTACC |
| | | TCCACCGCCTACATGGAACTGTCCAGCCTGAGATCTGAGGACACCGCCGTG |
| | | TACTACTGTGCCAGAGGCGGCTACTCTTACGGCTCCCTGGATTATTGGGGC |
| | | CAGGGCACACTGGTCACCGTGTCCTCTGGTGGCGGAGGAAGCGGAGGCGGA |
| | | GGTTCTGGCGGCGGAGGATCTGACATCCAGATGACCCAGTCTCCATCCTCT |
| | | CTGTCCGCCTCTGTGGGCGACAGAGTGACCATCACCTGTCGGGCCTCTCAG |
| | | TCCATCATCTCCGACCTGAACTGGTATCAGCAGAAGCCCGGCAAGGCCCCT |
| | | AAGCTGCTGATCCATGCTGCTTCCAGTCTGCAGTCTGGCGTGCCCTCTAGA |
| | | TTCTCCGGCTCTGGCTCTGGCACCGACTTTACCCTGACAATCTCCAGCCTG |
| | | CAGCCTGAGGACTTCGCCACCTACTACTGCCAGCAGTCCTACAGCACCCCT |
| | | TACACCTTTGGCCAGGGCACCAAGCTGGAAATCAAG |
| SEQ ID | aCCR2 | ATGGAAACCGACACACTGCTGCTGTGGGTGCTGCTCTTGTGGGTGCCAGGA |
| NO: | Hum1 VH- | TCTACAGGAGAAGTGCAGCTGGTGGAAAGCGGTGGCGGTCTGGTGCAGCCT |
| 305 | hCH1- | GGCGGTTCCCTCCGGCTGTCATGTGCTGCCTCCGGATTCTCGTTCAACGCG |
| | hFc_Knob- | TATGCCATGAACTGGGTGAGGCAGGCCCCGGGAAAGGGACTGGAATGGGTG |
| | 3 x 4GS- | GGGAGAATCCGGACCAAGAACAATAACTACGCTACTTACTACGCCGACTCC |
| | BH794_VH- | GTCAAGGACCGGTTCACAATTTCCCGGGACGACAGCAAGAGCTCACTGTAC |
| | 3 x 4GS- | CTTCAGATGAACAGCCTCAAAACCGAGGACACCGCCGTGTACTACTGTGCC |
| | BH794_VL | ACCTTTTACGGCAACGGTGTGTGGGGACAGGGAACAACTGTCACCGTGTCC |
| | | TCCGCTTCTACCAAGGGACCCAGCGTGTTCCCTCTGGCTCCTTCCAGCAAG |
| | | TCTACCTCTGGCGGAACAGCTGCTCTGGGCTGCCTGGTCAAGGACTACTTT |
| | | CCTGAGCCTGTGACCGTGTCTTGGAACTCTGGCGCTCTGACATCCGGCGTG |
| | | CACACATTTCCAGCTGTGCTGCAGTCCTCCGGCCTGTACTCTCTGTCCTCT |
| | | GTCGTGACCGTGCCTTCCAGCTCTCTGGGAACCCAGACCTACATCTGCAAT |
| | | GTGAACCACAAGCCTTCCAACACCAAGGTGGACAAGAGAGTGGAACCCAAG |
| | | TCCTGCGATAAGACCCACACATGTCCTCCATGCCCTGCCCCTGAGCTGCTG |
| | | GGCGGACCTTCCGTGTTCCTGTTCCCTCCAAAGCCCAAGGACACCCTGATG |
| | | ATCAGCCGGACCCCTGAAGTGACCTGCGTGGTGGTGGATGTGTCCCACGAG |
| | | GATCCCGAAGTGAAGTTCAATTGGTACGTGGACGGCGTGGAAGTGCACAAC |
| | | GCCAAGACCAAGCCCAGAGAGGAACAGTACAACAGCACCTACCGGGTGGTG |
| | | TCCGTGCTGACCGTGCTGCATCAGGACTGGCTGAACGGCAAAGAGTACAAG |
| | | TGCAAGGTGTCCAACAAGGCCCTGCCTGCCCCTATCGAGAAAACCATCAGC |
| | | AAGGCCAAGGGCCAGCCCCGCGAACCTCAGGTGTACACACTGCCTCCCTGC |
| | | CGGGAAGAGATGACCAAGAACCAGGTGTCCCTGTGGTGCCTGGTCAAGGGC |
| | | TTCTACCCCTCCGATATCGCCGTGGAATGGGAGAGCAACGGCCAGCCCGAG |
| | | AACAACTACAAGACCACCCCTCCCGTGCTGGACAGCGACGGCAGCTTCTTC |
| | | CTGTACTCCAAACTGACCGTGGACAAGAGCCGGTGGCAGCAGGGCAATGTG |
| | | TTCAGCTGTAGCGTGATGCACGAGGCCCTGCACAACCACTACACCCAGAAG |
| | | TCCCTGAGCCTGTCTCCTGGCAAAGGTGGCGGAGGAAGCGGAGGCGGAGGT |
| | | TCTGGCGGCGGAGGATCTCAGGTGCAGCTGGTTCAGTCTGGCGCCGAAGTG |
| | | AAGAAACCTGGCTCCTCCGTGAAGGTGTCCTGCAAGGCTTCTGGCGGCACC |
| | | TTCTCCTCTTACGCCATCTCCTGGGTCCGACAGGCTCCTGGACAAGGCTTG |
| | | GAATGGATGGGCGGCATCATCCCCATCTTCGGCACCGCCAATTACGCCCAG |
| | | AAATTCCAGGGCAGAGTGACCATCACCGCCGACAAGTCTACCTCCACCGCC |
| | | TACATGGAACTGTCCAGCCTGAGATCTGAGGACACCGCCGTGTACTACTGT |
| | | GCCAGAGGCGGCTACTCTTACGGCTCCCTGGATTATTGGGGCCAGGGCACA |
| | | CTGGTCACCGTGTCCTCTGGTGGCGGAGGAAGCGGAGGCGGAGGTTCTGGC |
| | | GGCGGAGGATCTGACATCCAGATGACCCAGTCTCCATCCTCTCTGTCCGCC |
| | | TCTGTGGGCGACAGAGTGACCATCACCTGTCGGGCCTCTCAGTCCATCATC |
| | | TCCGACCTGAACTGGTATCAGCAGAAGCCCGGCAAGGCCCCTAAGCTGCTG |
| | | ATCCATGCTGCTTCCAGTCTGCAGTCTGGCGTGCCCTCTAGATTCTCCGGC |
| | | TCTGGCTCTGGCACCGACTTTACCCTGACAATCTCCAGCCTGCAGCCTGAG |
| | | GACTTCGCCACCTACTACTGCCAGCAGTCCTACAGCACCCCTTACACCTTT |
| | | GGCCAGGGCACCAAGCTGGAAATCAAG |
| SEQ ID | aCSF1R | ATGGAAACCGACACACTGCTGCTGTGGGTGCTGCTCTTGTGGGTGCCAGGA |
| NO: | BI117 VL- | TCTACAGGACAACTGGTGCTGACCCAGCCCCCTTCCGTCTCCGGCGCCCCG |
| 306 | hCLIg_v1- | GGCCAGCGCGTCACTATCTCCTGTACCGGCAGCTCCAGCAACATCGGAGCA |
| | 3 x 4GS-IL2 | GGATACGACGTGCACTGGTACCAACAGCTCCCCGGAACCGCGCCTAAGCTG |
| | | CTGATCTACGGGAACTCTAACCGGCCGTCCGGCGTGCCTGACCGCTTCAGC |
| | | GGATCGAAGTCCGGCACCAGCGCCTCCCTGGCCATCACTGGACTGCAGGCC |
| | | GAGGACGAAGCCGATTACTACTGCCAGTCCTACGACTCCTCCCTGTCCGAG |
| | | GAAGTGTTCGGAGGCGGGACGAAGCTCACCGTCCTGGGACAACCTAAGGCC |
| | | AATCCTACCGTGACACTGTTCCCTCCATCCTCCGAGGAACTGCAGGCCAAC |
| | | AAGGCTACCCTCGTGTGCCTGATCTCCGACTTTTACCCTGGCGCTGTGACC |
| | | GTGGCCTGGAAGGCTGATGGATCTCCTGTGAAGGCTGGCGTGGAAACCACC |
| | | AAGCCTTCCAAGCAGTCCAACAACAAATACGCCGCCTCCTCCTACCTGTCT |
| | | CTGACCCCTGAACAGTGGAAGTCCCACCGGTCCTACAGCTGCCAAGTGACC |
| | | CATGAGGGCTCCACCGTGGAAAAGACCGTGGCTCCTACCGAGTGCTCCGGT |
| | | GGCGGAGGAAGCGGAGGCGGAGGTTCTGGCGGCGGAGGATCTGCCCCTACC |
| | | AGCAGCAGCACCAAGAAAACCCAGCTCCAGCTCGAGCACCTCCTGCTGGAC |
| | | CTGCAGATGATCCTGAACGGCATCAACAACTACAAGAACCCCAAGCTGACC |
| | | CGGATGCTGACCTTCAAGTTCTACATGCCCAAGAAGGCCACCGAGCTGAAG |
| | | CACCTCCAGTGCCTGGAAGAGGAACTGAAGCCCCTGGAAGAAGTGCTGAAC |
| | | CTGGCCCAGAGCAAGAACTTCCACCTGAGGCCCAGGGACCTGATCAGCAAC |
| | | ATCAACGTGATCGTGCTGGAACTGAAAGGCAGCGAGACAACCTTCATGTGC |
| | | GAGTACGCCGACGAGACAGCCACCATCGTGGAATTTCTGAACCGGTGGATC |
| | | ACCTTCTGCCAGAGCATCATCAGCACCCTGACA |
| SEQ ID | aCSF1R | ATGGAAACCGACACACTGCTGCTGTGGGTGCTGCTCTTGTGGGTGCCAGGA |
| NO: | BI117 VL- | TCTACAGGACAACTGGTGCTGACCCAGCCCCCTTCCGTCTCCGGCGCCCCG |
| 307 | hCLIg_v1- | GGCCAGCGCGTCACTATCTCCTGTACCGGCAGCTCCAGCAACATCGGAGCA |
| | IL2 | GGATACGACGTGCACTGGTACCAACAGCTCCCCGGAACCGCGCCTAAGCTG |
| | | CTGATCTACGGGAACTCTAACCGGCCGTCCGGCGTGCCTGACCGCTTCAGC |
| | | GGATCGAAGTCCGGCACCAGCGCCTCCCTGGCCATCACTGGACTGCAGGCC |
| | | GAGGACGAAGCCGATTACTACTGCCAGTCCTACGACTCCTCCCTGTCCGAG |
| | | GAAGTGTTCGGAGGCGGGACGAAGCTCACCGTCCTGGGACAACCTAAGGCC |
| | | AATCCTACCGTGACACTGTTCCCTCCATCCTCCGAGGAACTGCAGGCCAAC |
| | | AAGGCTACCCTCGTGTGCCTGATCTCCGACTTTTACCCTGGCGCTGTGACC |
| | | GTGGCCTGGAAGGCTGATGGATCTCCTGTGAAGGCTGGCGTGGAAACCACC |
| | | AAGCCTTCCAAGCAGTCCAACAACAAATACGCCGCCTCCTCCTACCTGTCT |
| | | CTGACCCCTGAACAGTGGAAGTCCCACCGGTCCTACAGCTGCCAAGTGACC |
| | | CATGAGGGCTCCACCGTGGAAAAGACCGTGGCTCCTACCGAGTGCTCCGCC |
| | | CCTACCAGCAGCAGCACCAAGAAAACCCAGCTCCAGCTCGAGCACCTCCTG |
| | | CTGGACCTGCAGATGATCCTGAACGGCATCAACAACTACAAGAACCCCAAG |
| | | CTGACCCGGATGCTGACCTTCAAGTTCTACATGCCCAAGAAGGCCACCGAG |
| | | CTGAAGCACCTCCAGTGCCTGGAAGAGGAACTGAAGCCCCTGGAAGAAGTG |
| | | CTGAACCTGGCCCAGAGCAAGAACTTCCACCTGAGGCCCAGGGACCTGATC |
| | | AGCAACATCAACGTGATCGTGCTGGAACTGAAAGGCAGCGAGACAACCTTC |
| | | ATGTGCGAGTACGCCGACGAGACAGCCACCATCGTGGAATTTCTGAACCGG |
| | | TGGATCACCTTCTGCCAGAGCATCATCAGCACCCTGACA |
| SEQ ID | aCSF1R | ATGGAAACCGACACACTGCTGCTGTGGGTGCTGCTCTTGTGGGTGCCAGGA |
| NO: | BI123 VL- | TCTACAGGATCCCTCGTGCTGACCCAACCTCCTTCAGTCTCCGTGGCCCCC |
| 308 | hCLIg_v1- | GGCAAGACCGCCCGCATCACCTGTGGCGGTAACAACATCGGCTCCAAGTCA |
| | 3 x 4GS-IL2 | GTCCACTGGTACCAGCAGAAGCCGGGCCAAGCTCCCGTGCTGGTCGTGTAC |
| | | GACGACTCAGACCGCCCCAGCGGTATCCCTGAGAGATTTAGCGGATCCAAC |
| | | TCCGGGAACACCGCTACCCTGACCATCTCAAGGGTGGAGGCCGGAGACGAA |
| | | GCCGACTACTACTGCCAGGTGTGGGATTCCAGCTCCGACCACGTGGTGTTC |
| | | GGCGGCGGCACTCAGCTCACCGTGCTGGGACAACCTAAGGCCAATCCTACC |
| | | GTGACACTGTTCCCTCCATCCTCCGAGGAACTGCAGGCCAACAAGGCTACC |
| | | CTCGTGTGCCTGATCTCCGACTTTTACCCTGGCGCTGTGACCGTGGCCTGG |
| | | AAGGCTGATGGATCTCCTGTGAAGGCTGGCGTGGAAACCACCAAGCCTTCC |
| | | AAGCAGTCCAACAACAAATACGCCGCCTCCTCCTACCTGTCTCTGACCCCT |
| | | GAACAGTGGAAGTCCCACCGGTCCTACAGCTGCCAAGTGACCCATGAGGGC |
| | | TCCACCGTGGAAAAGACCGTGGCTCCTACCGAGTGCTCCGGTGGCGGAGGA |
| | | AGCGGAGGCGGAGGTTCTGGCGGCGGAGGATCTGCCCCTACCAGCAGCAGC |
| | | ACCAAGAAAACCCAGCTCCAGCTCGAGCACCTCCTGCTGGACCTGCAGATG |
| | | ATCCTGAACGGCATCAACAACTACAAGAACCCCAAGCTGACCCGGATGCTG |
| | | ACCTTCAAGTTCTACATGCCCAAGAAGGCCACCGAGCTGAAGCACCTCCAG |
| | | TGCCTGGAAGAGGAACTGAAGCCCCTGGAAGAAGTGCTGAACCTGGCCCAG |
| | | AGCAAGAACTTCCACCTGAGGCCCAGGGACCTGATCAGCAACATCAACGTG |
| | | ATCGTGCTGGAACTGAAAGGCAGCGAGACAACCTTCATGTGCGAGTACGCC |
| | | GACGAGACAGCCACCATCGTGGAATTTCTGAACCGGTGGATCACCTTCTGC |
| | | CAGAGCATCATCAGCACCCTGACA |
| SEQ ID | aCSF1R | ATGGAAACCGACACACTGCTGCTGTGGGTGCTGCTCTTGTGGGTGCCAGGA |
| NO: | BI123 VL- | TCTACAGGATCCCTCGTGCTGACCCAACCTCCTTCAGTCTCCGTGGCCCCC |
| 309 | hCLIg_v1- | GGCAAGACCGCCCGCATCACCTGTGGCGGTAACAACATCGGCTCCAAGTCA |
| | IL2 | GTCCACTGGTACCAGCAGAAGCCGGGCCAAGCTCCCGTGCTGGTCGTGTAC |
| | | GACGACTCAGACCGCCCCAGCGGTATCCCTGAGAGATTTAGCGGATCCAAC |
| | | TCCGGGAACACCGCTACCCTGACCATCTCAAGGGTGGAGGCCGGAGACGAA |
| | | GCCGACTACTACTGCCAGGTGTGGGATTCCAGCTCCGACCACGTGGTGTTC |
| | | GGCGGCGGCACTCAGCTCACCGTGCTGGGACAACCTAAGGCCAATCCTACC |
| | | GTGACACTGTTCCCTCCATCCTCCGAGGAACTGCAGGCCAACAAGGCTACC |
| | | CTCGTGTGCCTGATCTCCGACTTTTACCCTGGCGCTGTGACCGTGGCCTGG |
| | | AAGGCTGATGGATCTCCTGTGAAGGCTGGCGTGGAAACCACCAAGCCTTCC |
| | | AAGCAGTCCAACAACAAATACGCCGCCTCCTCCTACCTGTCTCTGACCCCT |
| | | GAACAGTGGAAGTCCCACCGGTCCTACAGCTGCCAAGTGACCCATGAGGGC |
| | | TCCACCGTGGAAAAGACCGTGGCTCCTACCGAGTGCTCCGCCCCTACCAGC |
| | | AGCAGCACCAAGAAAACCCAGCTCCAGCTCGAGCACCTCCTGCTGGACCTG |
| | | CAGATGATCCTGAACGGCATCAACAACTACAAGAACCCCAAGCTGACCCGG |
| | | ATGCTGACCTTCAAGTTCTACATGCCCAAGAAGGCCACCGAGCTGAAGCAC |
| | | CTCCAGTGCCTGGAAGAGGAACTGAAGCCCCTGGAAGAAGTGCTGAACCTG |
| | | GCCCAGAGCAAGAACTTCCACCTGAGGCCCAGGGACCTGATCAGCAACATC |
| | | AACGTGATCGTGCTGGAACTGAAAGGCAGCGAGACAACCTTCATGTGCGAG |
| | | TACGCCGACGAGACAGCCACCATCGTGGAATTTCTGAACCGGTGGATCACC |
| | | TTCTGCCAGAGCATCATCAGCACCCTGACA |
| SEQ ID | aCCR2 | ATGGAAACCGACACACTGCTGCTGTGGGTGCTGCTCTTGTGGGTGCCAGGA |
| NO: | VL- | TCTACAGGAGACGTCGTCATGACCCAATCCCCGCTCTCCCTGCCGGTGACT |
| 310 | hCLIg_vk- | CTCGGCCAGCCGGCGTCCATCTCGTGCAAGTCCTCCCAATCCCTGCTGGAT |
| | 3 x 4GS-IL2 | TCGGATGGGAAGACTTTCCTGAACTGGCTGCAGCAACGGCCCGGCCAGTCG |
| | | CCACGGCGGCTGATCTACCTGGTGTCCAAGCTGGATTCAGGTGTGCCTGAT |
| | | AGATTCTCCGGCAGCGGATCGGGGACCGACTTCACGCTGAAGATCTCCAGA |
| | | GTGGAAGCAGAGGACGTGGGCGTCTATTACTGTTGGCAGGGGACTCATTTC |
| | | CCCTATACCTTTGGAGGTGGTACTAAGGTGGAAATTAAGAGAACAGTGGCC |
| | | GCTCCTTCCGTGTTCATCTTCCCACCCTCCGACGAGCAGCTGAAGTCCGGC |
| | | ACCGCTTCTGTCGTGTGCCTGCTCAACAACTTCTACCCTCGGGAAGCCAAG |
| | | GTGCAGTGGAAGGTGGACAACGCCCTGCAGTCCGGCAACTCCCAGGAATCC |
| | | GTCACCGAGCAGGACTCCAAGGACAGCACCTACTCCCTGTCCTCCACCCTG |
| | | ACCCTGTCCAAGGCCGACTACGAGAAGCACAAGGTGTACGCCTGCGAAGTG |
| | | ACCCACCAGGGCCTGAGCAGCCCCGTGACCAAGTCCTTCAACCGGGGCGAG |
| | | TGCGGTGGCGGAGGAAGCGGAGGCGGAGGTTCTGGCGGCGGAGGATCTGCC |
| | | CCTACCAGCAGCAGCACCAAGAAAACCCAGCTCCAGCTCGAGCACCTCCTG |
| | | CTGGACCTGCAGATGATCCTGAACGGCATCAACAACTACAAGAACCCCAAG |
| | | CTGACCCGGATGCTGACCTTCAAGTTCTACATGCCCAAGAAGGCCACCGAG |
| | | CTGAAGCACCTCCAGTGCCTGGAAGAGGAACTGAAGCCCCTGGAAGAAGTG |
| | | CTGAACCTGGCCCAGAGCAAGAACTTCCACCTGAGGCCCAGGGACCTGATC |
| | | AGCAACATCAACGTGATCGTGCTGGAACTGAAAGGCAGCGAGACAACCTTC |
| | | ATGTGCGAGTACGCCGACGAGACAGCCACCATCGTGGAATTTCTGAACCGG |
| | | TGGATCACCTTCTGCCAGAGCATCATCAGCACCCTGACA |
| SEQ ID | aCCR2 | ATGGAAACCGACACACTGCTGCTGTGGGTGCTGCTCTTGTGGGTGCCAGGA |
| NO: | VL- | TCTACAGGAGACGTCGTCATGACCCAATCCCCGCTCTCCCTGCCGGTGACT |
| 311 | hCLIg_vk- | CTCGGCCAGCCGGCGTCCATCTCGTGCAAGTCCTCCCAATCCCTGCTGGAT |
| | IL2 | TCGGATGGGAAGACTTTCCTGAACTGGCTGCAGCAACGGCCCGGCCAGTCG |
| | | CCACGGCGGCTGATCTACCTGGTGTCCAAGCTGGATTCAGGTGTGCCTGAT |
| | | AGATTCTCCGGCAGCGGATCGGGGACCGACTTCACGCTGAAGATCTCCAGA |
| | | GTGGAAGCAGAGGACGTGGGCGTCTATTACTGTTGGCAGGGGACTCATTTC |
| | | CCCTATACCTTTGGAGGTGGTACTAAGGTGGAAATTAAGAGAACAGTGGCC |
| | | GCTCCTTCCGTGTTCATCTTCCCACCCTCCGACGAGCAGCTGAAGTCCGGC |
| | | ACCGCTTCTGTCGTGTGCCTGCTCAACAACTTCTACCCTCGGGAAGCCAAG |
| | | GTGCAGTGGAAGGTGGACAACGCCCTGCAGTCCGGCAACTCCCAGGAATCC |
| | | GTCACCGAGCAGGACTCCAAGGACAGCACCTACTCCCTGTCCTCCACCCTG |
| | | ACCCTGTCCAAGGCCGACTACGAGAAGCACAAGGTGTACGCCTGCGAAGTG |
| | | ACCCACCAGGGCCTGAGCAGCCCCGTGACCAAGTCCTTCAACCGGGGCGAG |
| | | TGCGCCCCTACCAGCAGCAGCACCAAGAAAACCCAGCTCCAGCTCGAGCAC |
| | | CTCCTGCTGGACCTGCAGATGATCCTGAACGGCATCAACAACTACAAGAAC |
| | | CCCAAGCTGACCCGGATGCTGACCTTCAAGTTCTACATGCCCAAGAAGGCC |
| | | ACCGAGCTGAAGCACCTCCAGTGCCTGGAAGAGGAACTGAAGCCCCTGGAA |
| | | GAAGTGCTGAACCTGGCCCAGAGCAAGAACTTCCACCTGAGGCCCAGGGAC |
| | | CTGATCAGCAACATCAACGTGATCGTGCTGGAACTGAAAGGCAGCGAGACA |
| | | ACCTTCATGTGCGAGTACGCCGACGAGACAGCCACCATCGTGGAATTTCTG |
| | | AACCGGTGGATCACCTTCTGCCAGAGCATCATCAGCACCCTGACA |
| SEQ ID | aCSF1R | ATGGAAACCGACACACTGCTGCTGTGGGTGCTGCTCTTGTGGGTGCCAGGA |
| NO: | BI117 VH- | TCTACAGGAGAAGTGCAGCTGGTCGAAAGCGGAGGGGGTCTGGTCCAGCCT |
| 312 | hCH1- | GGAGGGTCCCTGCGGCTGTCCTGCGCCGCTTCCGGCTTCACCTTCTCCAAC |
| | hFc_Hole- | GATGAAATGAACTGGATCCGCCAAGCCCCTGGAAAGGGCCTGGAATGGGTG |
| | 3 x 4GS- | TCGTACATCTCGAGCAGGTCCAGCACTATCTACTATGCCGACGCGGTGAAG |
| | TGFbR2 | GGACGCTTCACTATCAGCCGCGATAACGCACGCAATTCCCTCTACCTGCAG |
| | | ATGAACTCGCTCAGAGCTGAGGATACTGCCGTCTACTACTGCGCCCGCGGC |
| | | GTGGACGGCGATTTTTCCTTCGATTACTGGGGACAGGGCACTCTTGTCACC |
| | | GTGTCAAGCGCTTCTACCAAGGGACCCAGCGTGTTCCCTCTGGCTCCTTCC |
| | | AGCAAGTCTACCTCTGGCGGAACAGCTGCTCTGGGCTGCCTGGTCAAGGAC |
| | | TACTTTCCTGAGCCTGTGACCGTGTCTTGGAACTCTGGCGCTCTGACATCC |
| | | GGCGTGCACACATTTCCAGCTGTGCTGCAGTCCTCCGGCCTGTACTCTCTG |
| | | TCCTCTGTCGTGACCGTGCCTTCCAGCTCTCTGGGAACCCAGACCTACATC |
| | | TGCAATGTGAACCACAAGCCTTCCAACACCAAGGTGGACAAGAGAGTGGAA |
| | | CCCAAGTCCTGCGATAAGACCCACACCTGTCCTCCCTGCCCTGCCCCTGAA |
| | | CTGCTGGGCGGACCTAGCGTGTTCCTGTTCCCTCCAAAGCCCAAGGACACC |
| | | CTGATGATCAGCCGGACCCCTGAAGTGACCTGCGTGGTGGTGGATGTGTCC |
| | | CACGAGGATCCCGAAGTGAAGTTCAATTGGTACGTGGACGGCGTGGAAGTG |
| | | CACAACGCCAAGACCAAGCCCAGAGAGGAACAGTACAACAGCACCTACCGG |
| | | GTGGTGTCCGTGCTGACCGTGCTGCACCAGGACTGGCTGAACGGCAAAGAG |
| | | TACAAGTGCAAGGTGTCCAACAAGGCCCTGCCAGCCCCTATCGAGAAAACC |
| | | ATCAGCAAGGCCAAGGGCCAGCCTAGAGAGCCTCAGGTCTGCACCCTGCCT |
| | | CCCAGCCGGGAAGAGATGACCAAGAACCAGGTGTCCCTGAGCTGCGCCGTG |
| | | AAGGGCTTCTACCCCTCCGATATCGCCGTGGAATGGGAGAGCAACGGCCAG |
| | | CCCGAGAACAACTACAAGACCACCCCTCCCGTGCTGGACAGCGACGGCAGC |
| | | TTCTTCCTGGTGTCCAAACTGACCGTGGACAAGAGCCGGTGGCAGCAGGGC |
| | | AATGTGTTCAGCTGTAGCGTGATGCACGAGGCCCTGCACAACCACTACACC |
| | | CAGAAGTCTCTGAGCCTGAGCCCTGGCAAAGGTGGCGGAGGAAGCGGAGGC |
| | | GGAGGTTCTGGCGGCGGAGGATCTATTCCTCCACACGTGCAAAAGTCGGTG |
| | | AACAATGATATGATCGTCACCGACAACAACGGAGCCGTGAAGTTTCCTCAG |
| | | CTGTGCAAGTTTTGTGATGTCCGCTTCAGCACCTGTGACAACCAGAAGTCA |
| | | TGCATGTCGAACTGTTCGATCACCAGCATTTGCGAAAAGCCTCAAGAGGTG |
| | | TGCGTGGCCGTGTGGCGCAAGAACGACGAAAACATCACTCTGGAAACCGTG |
| | | TGCCATGATCCTAAGCTCCCGTACCATGACTTCATTCTTGAAGATGCAGCC |
| | | TCCCCCAAGTGCATCATGAAGGAGAAGAAGAAGCCTGGCGAAACCTTCTTC |
| | | ATGTGCTCATGCAGCTCCGACGAATGTAACGACAACATCATCTTCTCAGAG |
| | | GAATACAACACCTCAAACCCGGAC |
| SEQ ID | aCSF1R | ATGGAAACCGACACACTGCTGCTGTGGGTGCTGCTCTTGTGGGTGCCAGGA |
| NO: | BI123 VH- | TCTACAGGACAGGTGCAGCTTGTGCAAAGCGGGGGAGGCGTGGTGCAGCCG |
| 313 | hCH1- | GGACGGAGCCTGCGGCTGTCGTGCGCCGCGAGCGGTTTCACCTTCTCCTCC |
| | hFc_Hole- | TACGCTATGCACTGGGTCCGCCAGGCGCCGGGCAAGGGCCTCGAATGGGTG |
| | 3 x 4GS- | GCCGTGATCAGCTACGATGGCTCCAACAAGTACTACGCCGACTCAGTGAAG |
| | TGFbR2 | GGAAGGTTCACCATCTCAAGAGATAACAGCAAGAACACGCTGTACCTCCAG |
| | | ATGAACTCCCTCCGGGCCGAAGACACAGCCGTCTACTACTGTGCGAGAGAG |
| | | CTCGATTACGGCGATTACGGCCTGGACGTGTGGGGCAAGGGAACCACGGTG |
| | | ACCGTCTCCTCCGCTTCTACCAAGGGACCCAGCGTGTTCCCTCTGGCTCCT |
| | | TCCAGCAAGTCTACCTCTGGCGGAACAGCTGCTCTGGGCTGCCTGGTCAAG |
| | | GACTACTTTCCTGAGCCTGTGACCGTGTCTTGGAACTCTGGCGCTCTGACA |
| | | TCCGGCGTGCACACATTTCCAGCTGTGCTGCAGTCCTCCGGCCTGTACTCT |
| | | CTGTCCTCTGTCGTGACCGTGCCTTCCAGCTCTCTGGGAACCCAGACCTAC |
| | | ATCTGCAATGTGAACCACAAGCCTTCCAACACCAAGGTGGACAAGAGAGTG |
| | | GAACCCAAGTCCTGCGATAAGACCCACACCTGTCCTCCCTGCCCTGCCCCT |
| | | GAACTGCTGGGCGGACCTAGCGTGTTCCTGTTCCCTCCAAAGCCCAAGGAC |
| | | ACCCTGATGATCAGCCGGACCCCTGAAGTGACCTGCGTGGTGGTGGATGTG |
| | | TCCCACGAGGATCCCGAAGTGAAGTTCAATTGGTACGTGGACGGCGTGGAA |
| | | GTGCACAACGCCAAGACCAAGCCCAGAGAGGAACAGTACAACAGCACCTAC |
| | | CGGGTGGTGTCCGTGCTGACCGTGCTGCACCAGGACTGGCTGAACGGCAAA |
| | | GAGTACAAGTGCAAGGTGTCCAACAAGGCCCTGCCAGCCCCTATCGAGAAA |
| | | ACCATCAGCAAGGCCAAGGGCCAGCCTAGAGAGCCTCAGGTCTGCACCCTG |
| | | CCTCCCAGCCGGGAAGAGATGACCAAGAACCAGGTGTCCCTGAGCTGCGCC |
| | | GTGAAGGGCTTCTACCCCTCCGATATCGCCGTGGAATGGGAGAGCAACGGC |
| | | CAGCCCGAGAACAACTACAAGACCACCCCTCCCGTGCTGGACAGCGACGGC |
| | | AGCTTCTTCCTGGTGTCCAAACTGACCGTGGACAAGAGCCGGTGGCAGCAG |
| | | GGCAATGTGTTCAGCTGTAGCGTGATGCACGAGGCCCTGCACAACCACTAC |
| | | ACCCAGAAGTCTCTGAGCCTGAGCCCTGGCAAAGGTGGCGGAGGAAGCGGA |
| | | GGCGGAGGTTCTGGCGGCGGAGGATCTATTCCTCCACACGTGCAAAAGTCG |
| | | GTGAACAATGATATGATCGTCACCGACAACAACGGAGCCGTGAAGTTTCCT |
| | | CAGCTGTGCAAGTTTTGTGATGTCCGCTTCAGCACCTGTGACAACCAGAAG |
| | | TCATGCATGTCGAACTGTTCGATCACCAGCATTTGCGAAAAGCCTCAAGAG |
| | | GTGTGCGTGGCCGTGTGGCGCAAGAACGACGAAAACATCACTCTGGAAACC |
| | | GTGTGCCATGATCCTAAGCTCCCGTACCATGACTTCATTCTTGAAGATGCA |
| | | GCCTCCCCCAAGTGCATCATGAAGGAGAAGAAGAAGCCTGGCGAAACCTTC |
| | | TTCATGTGCTCATGCAGCTCCGACGAATGTAACGACAACATCATCTTCTCA |
| | | GAGGAATACAACACCTCAAACCCGGAC |
| SEQ ID | aCCR2 | ATGGAAACCGACACACTGCTGCTGTGGGTGCTGCTCTTGTGGGTGCCAGGA |
| NO: | Hum1 VH- | TCTACAGGAGAAGTGCAGCTGGTGGAAAGCGGTGGCGGTCTGGTGCAGCCT |
| 314 | hCH1- | GGCGGTTCCCTCCGGCTGTCATGTGCTGCCTCCGGATTCTCGTTCAACGCG |
| | hFc_Knob- | TATGCCATGAACTGGGTGAGGCAGGCCCCGGGAAAGGGACTGGAATGGGTG |
| | 3 x 4GS- | GGGAGAATCCGGACCAAGAACAATAACTACGCTACTTACTACGCCGACTCC |
| | TGFbR2 | GTCAAGGACCGGTTCACAATTTCCCGGGACGACAGCAAGAGCTCACTGTAC |
| | | CTTCAGATGAACAGCCTCAAAACCGAGGACACCGCCGTGTACTACTGTGCC |
| | | ACCTTTTACGGCAACGGTGTGTGGGGACAGGGAACAACTGTCACCGTGTCC |
| | | TCCGCTTCTACCAAGGGACCCAGCGTGTTCCCTCTGGCTCCTTCCAGCAAG |
| | | TCTACCTCTGGCGGAACAGCTGCTCTGGGCTGCCTGGTCAAGGACTACTTT |
| | | CCTGAGCCTGTGACCGTGTCTTGGAACTCTGGCGCTCTGACATCCGGCGTG |
| | | CACACATTTCCAGCTGTGCTGCAGTCCTCCGGCCTGTACTCTCTGTCCTCT |
| | | GTCGTGACCGTGCCTTCCAGCTCTCTGGGAACCCAGACCTACATCTGCAAT |
| | | GTGAACCACAAGCCTTCCAACACCAAGGTGGACAAGAGAGTGGAACCCAAG |
| | | TCCTGCGATAAGACCCACACATGTCCTCCATGCCCTGCCCCTGAGCTGCTG |
| | | GGCGGACCTTCCGTGTTCCTGTTCCCTCCAAAGCCCAAGGACACCCTGATG |
| | | ATCAGCCGGACCCCTGAAGTGACCTGCGTGGTGGTGGATGTGTCCCACGAG |
| | | GATCCCGAAGTGAAGTTCAATTGGTACGTGGACGGCGTGGAAGTGCACAAC |
| | | GCCAAGACCAAGCCCAGAGAGGAACAGTACAACAGCACCTACCGGGTGGTG |
| | | TCCGTGCTGACCGTGCTGCATCAGGACTGGCTGAACGGCAAAGAGTACAAG |
| | | TGCAAGGTGTCCAACAAGGCCCTGCCTGCCCCTATCGAGAAAACCATCAGC |
| | | AAGGCCAAGGGCCAGCCCCGCGAACCTCAGGTGTACACACTGCCTCCCTGC |
| | | CGGGAAGAGATGACCAAGAACCAGGTGTCCCTGTGGTGCCTGGTCAAGGGC |
| | | TTCTACCCCTCCGATATCGCCGTGGAATGGGAGAGCAACGGCCAGCCCGAG |
| | | AACAACTACAAGACCACCCCTCCCGTGCTGGACAGCGACGGCAGCTTCTTC |
| | | CTGTACTCCAAACTGACCGTGGACAAGAGCCGGTGGCAGCAGGGCAATGTG |
| | | TTCAGCTGTAGCGTGATGCACGAGGCCCTGCACAACCACTACACCCAGAAG |
| | | TCCCTGAGCCTGTCTCCTGGCAAAGGTGGCGGAGGAAGCGGAGGCGGAGGT |
| | | TCTGGCGGCGGAGGATCTATTCCTCCACACGTGCAAAAGTCGGTGAACAAT |
| | | GATATGATCGTCACCGACAACAACGGAGCCGTGAAGTTTCCTCAGCTGTGC |
| | | AAGTTTTGTGATGTCCGCTTCAGCACCTGTGACAACCAGAAGTCATGCATG |
| | | TCGAACTGTTCGATCACCAGCATTTGCGAAAAGCCTCAAGAGGTGTGCGTG |
| | | GCCGTGTGGCGCAAGAACGACGAAAACATCACTCTGGAAACCGTGTGCCAT |
| | | GATCCTAAGCTCCCGTACCATGACTTCATTCTTGAAGATGCAGCCTCCCCC |
| | | AAGTGCATCATGAAGGAGAAGAAGAAGCCTGGCGAAACCTTCTTCATGTGC |
| | | TCATGCAGCTCCGACGAATGTAACGACAACATCATCTTCTCAGAGGAATAC |
| | | AACACCTCAAACCCGGAC |
| SEQ ID | aCSF1R | ATGGAAACCGACACACTGCTGCTGTGGGTGCTGCTCTTGTGGGTGCCAGGA |
| NO: | BI117 VH- | TCTACAGGAGAAGTGCAGCTGGTCGAAAGCGGAGGGGGTCTGGTCCAGCCT |
| 315 | 3 x 4GS- | GGAGGGTCCCTGCGGCTGTCCTGCGCCGCTTCCGGCTTCACCTTCTCCAAC |
| | aCSF1R | GATGAAATGAACTGGATCCGCCAAGCCCCTGGAAAGGGCCTGGAATGGGTG |
| | BI117 VL- | TCGTACATCTCGAGCAGGTCCAGCACTATCTACTATGCCGACGCGGTGAAG |
| | 4GS- | GGACGCTTCACTATCAGCCGCGATAACGCACGCAATTCCCTCTACCTGCAG |
| | hFc_Hole | ATGAACTCGCTCAGAGCTGAGGATACTGCCGTCTACTACTGCGCCCGCGGC |
| | | GTGGACGGCGATTTTTCCTTCGATTACTGGGGACAGGGCACTCTTGTCACC |
| | | GTGTCAAGCGGTGGCGGAGGAAGCGGAGGCGGAGGTTCTGGCGGCGGAGGA |
| | | TCTCAACTGGTGCTGACCCAGCCCCCTTCCGTCTCCGGCGCCCCGGGCCAG |
| | | CGCGTCACTATCTCCTGTACCGGCAGCTCCAGCAACATCGGAGCAGGATAC |
| | | GACGTGCACTGGTACCAACAGCTCCCCGGAACCGCGCCTAAGCTGCTGATC |
| | | TACGGGAACTCTAACCGGCCGTCCGGCGTGCCTGACCGCTTCAGCGGATCG |
| | | AAGTCCGGCACCAGCGCCTCCCTGGCCATCACTGGACTGCAGGCCGAGGAC |
| | | GAAGCCGATTACTACTGCCAGTCCTACGACTCCTCCCTGTCCGAGGAAGTG |
| | | TTCGGAGGCGGGACGAAGCTCACCGTCCTGGGTGGCGGAGGAAGCGATAAG |
| | | ACCCACACCTGTCCTCCCTGCCCTGCCCCTGAACTGCTGGGCGGACCTAGC |
| | | GTGTTCCTGTTCCCTCCAAAGCCCAAGGACACCCTGATGATCAGCCGGACC |
| | | CCTGAAGTGACCTGCGTGGTGGTGGATGTGTCCCACGAGGATCCCGAAGTG |
| | | AAGTTCAATTGGTACGTGGACGGCGTGGAAGTGCACAACGCCAAGACCAAG |
| | | CCCAGAGAGGAACAGTACAACAGCACCTACCGGGTGGTGTCCGTGCTGACC |
| | | GTGCTGCACCAGGACTGGCTGAACGGCAAAGAGTACAAGTGCAAGGTGTCC |
| | | AACAAGGCCCTGCCAGCCCCTATCGAGAAAACCATCAGCAAGGCCAAGGGC |
| | | CAGCCTAGAGAGCCTCAGGTCTGCACCCTGCCTCCCAGCCGGGAAGAGATG |
| | | ACCAAGAACCAGGTGTCCCTGAGCTGCGCCGTGAAGGGCTTCTACCCCTCC |
| | | GATATCGCCGTGGAATGGGAGAGCAACGGCCAGCCCGAGAACAACTACAAG |
| | | ACCACCCCTCCCGTGCTGGACAGCGACGGCAGCTTCTTCCTGGTGTCCAAA |
| | | CTGACCGTGGACAAGAGCCGGTGGCAGCAGGGCAATGTGTTCAGCTGTAGC |
| | | GTGATGCACGAGGCCCTGCACAACCACTACACCCAGAAGTCTCTGAGCCTG |
| | | AGCCCTGGCAAA |
| SEQ ID | aCSF1R | ATGGAAACCGACACACTGCTGCTGTGGGTGCTGCTCTTGTGGGTGCCAGGA |
| NO: | BI123 VH- | TCTACAGGACAGGTGCAGCTTGTGCAAAGCGGGGGAGGCGTGGTGCAGCCG |
| 316 | 3 x 4GS- | GGACGGAGCCTGCGGCTGTCGTGCGCCGCGAGCGGTTTCACCTTCTCCTCC |
| | aCSF1R | TACGCTATGCACTGGGTCCGCCAGGCGCCGGGCAAGGGCCTCGAATGGGTG |
| | BI123 VL- | GCCGTGATCAGCTACGATGGCTCCAACAAGTACTACGCCGACTCAGTGAAG |
| | 4GS- | GGAAGGTTCACCATCTCAAGAGATAACAGCAAGAACACGCTGTACCTCCAG |
| | hFc_Hole | ATGAACTCCCTCCGGGCCGAAGACACAGCCGTCTACTACTGTGCGAGAGAG |
| | | CTCGATTACGGCGATTACGGCCTGGACGTGTGGGGCAAGGGAACCACGGTG |
| | | ACCGTCTCCTCCGGTGGCGGAGGAAGCGGAGGCGGAGGTTCTGGCGGCGGA |
| | | GGATCTTCCCTCGTGCTGACCCAACCTCCTTCAGTCTCCGTGGCCCCCGGC |
| | | AAGACCGCCCGCATCACCTGTGGCGGTAACAACATCGGCTCCAAGTCAGTC |
| | | CACTGGTACCAGCAGAAGCCGGGCCAAGCTCCCGTGCTGGTCGTGTACGAC |
| | | GACTCAGACCGCCCCAGCGGTATCCCTGAGAGATTTAGCGGATCCAACTCC |
| | | GGGAACACCGCTACCCTGACCATCTCAAGGGTGGAGGCCGGAGACGAAGCC |
| | | GACTACTACTGCCAGGTGTGGGATTCCAGCTCCGACCACGTGGTGTTCGGC |
| | | GGCGGCACTCAGCTCACCGTGCTGGGTGGCGGAGGAAGCGATAAGACCCAC |
| | | ACCTGTCCTCCCTGCCCTGCCCCTGAACTGCTGGGCGGACCTAGCGTGTTC |
| | | CTGTTCCCTCCAAAGCCCAAGGACACCCTGATGATCAGCCGGACCCCTGAA |
| | | GTGACCTGCGTGGTGGTGGATGTGTCCCACGAGGATCCCGAAGTGAAGTTC |
| | | AATTGGTACGTGGACGGCGTGGAAGTGCACAACGCCAAGACCAAGCCCAGA |
| | | GAGGAACAGTACAACAGCACCTACCGGGTGGTGTCCGTGCTGACCGTGCTG |
| | | CACCAGGACTGGCTGAACGGCAAAGAGTACAAGTGCAAGGTGTCCAACAAG |
| | | GCCCTGCCAGCCCCTATCGAGAAAACCATCAGCAAGGCCAAGGGCCAGCCT |
| | | AGAGAGCCTCAGGTCTGCACCCTGCCTCCCAGCCGGGAAGAGATGACCAAG |
| | | AACCAGGTGTCCCTGAGCTGCGCCGTGAAGGGCTTCTACCCCTCCGATATC |
| | | GCCGTGGAATGGGAGAGCAACGGCCAGCCCGAGAACAACTACAAGACCACC |
| | | CCTCCCGTGCTGGACAGCGACGGCAGCTTCTTCCTGGTGTCCAAACTGACC |
| | | GTGGACAAGAGCCGGTGGCAGCAGGGCAATGTGTTCAGCTGTAGCGTGATG |
| | | CACGAGGCCCTGCACAACCACTACACCCAGAAGTCTCTGAGCCTGAGCCCT |
| | | GGCAAA |
| SEQ ID | aCSF1R | ATGGAAACCGACACACTGCTGCTGTGGGTGCTGCTCTTGTGGGTGCCAGGA |
| NO: | BI117 VH- | TCTACAGGAGAAGTGCAGCTGGTCGAAAGCGGAGGGGGTCTGGTCCAGCCT |
| 317 | 3 x 4GS- | GGAGGGTCCCTGCGGCTGTCCTGCGCCGCTTCCGGCTTCACCTTCTCCAAC |
| | aCSF1R | GATGAAATGAACTGGATCCGCCAAGCCCCTGGAAAGGGCCTGGAATGGGTG |
| | BI117 VL- | TCGTACATCTCGAGCAGGTCCAGCACTATCTACTATGCCGACGCGGTGAAG |
| | 4GS- | GGACGCTTCACTATCAGCCGCGATAACGCACGCAATTCCCTCTACCTGCAG |
| | hFc_Hole- | ATGAACTCGCTCAGAGCTGAGGATACTGCCGTCTACTACTGCGCCCGCGGC |
| | 3 x 4GS- | GTGGACGGCGATTTTTCCTTCGATTACTGGGGACAGGGCACTCTTGTCACC |
| | TGFbR2 | GTGTCAAGCGGTGGCGGAGGAAGCGGAGGCGGAGGTTCTGGCGGCGGAGGA |
| | | TCTCAACTGGTGCTGACCCAGCCCCCTTCCGTCTCCGGCGCCCCGGGCCAG |
| | | CGCGTCACTATCTCCTGTACCGGCAGCTCCAGCAACATCGGAGCAGGATAC |
| | | GACGTGCACTGGTACCAACAGCTCCCCGGAACCGCGCCTAAGCTGCTGATC |
| | | TACGGGAACTCTAACCGGCCGTCCGGCGTGCCTGACCGCTTCAGCGGATCG |
| | | AAGTCCGGCACCAGCGCCTCCCTGGCCATCACTGGACTGCAGGCCGAGGAC |
| | | GAAGCCGATTACTACTGCCAGTCCTACGACTCCTCCCTGTCCGAGGAAGTG |
| | | TTCGGAGGCGGGACGAAGCTCACCGTCCTGGGTGGCGGAGGAAGCGATAAG |
| | | ACCCACACCTGTCCTCCCTGCCCTGCCCCTGAACTGCTGGGCGGACCTAGC |
| | | GTGTTCCTGTTCCCTCCAAAGCCCAAGGACACCCTGATGATCAGCCGGACC |
| | | CCTGAAGTGACCTGCGTGGTGGTGGATGTGTCCCACGAGGATCCCGAAGTG |
| | | AAGTTCAATTGGTACGTGGACGGCGTGGAAGTGCACAACGCCAAGACCAAG |
| | | CCCAGAGAGGAACAGTACAACAGCACCTACCGGGTGGTGTCCGTGCTGACC |
| | | GTGCTGCACCAGGACTGGCTGAACGGCAAAGAGTACAAGTGCAAGGTGTCC |
| | | AACAAGGCCCTGCCAGCCCCTATCGAGAAAACCATCAGCAAGGCCAAGGGC |
| | | CAGCCTAGAGAGCCTCAGGTCTGCACCCTGCCTCCCAGCCGGGAAGAGATG |
| | | ACCAAGAACCAGGTGTCCCTGAGCTGCGCCGTGAAGGGCTTCTACCCCTCC |
| | | GATATCGCCGTGGAATGGGAGAGCAACGGCCAGCCCGAGAACAACTACAAG |
| | | ACCACCCCTCCCGTGCTGGACAGCGACGGCAGCTTCTTCCTGGTGTCCAAA |
| | | CTGACCGTGGACAAGAGCCGGTGGCAGCAGGGCAATGTGTTCAGCTGTAGC |
| | | GTGATGCACGAGGCCCTGCACAACCACTACACCCAGAAGTCTCTGAGCCTG |
| | | AGCCCTGGCAAAGGTGGCGGAGGAAGCGGAGGCGGAGGTTCTGGCGGCGGA |
| | | GGATCTATTCCTCCACACGTGCAAAAGTCGGTGAACAATGATATGATCGTC |
| | | ACCGACAACAACGGAGCCGTGAAGTTTCCTCAGCTGTGCAAGTTTTGTGAT |
| | | GTCCGCTTCAGCACCTGTGACAACCAGAAGTCATGCATGTCGAACTGTTCG |
| | | ATCACCAGCATTTGCGAAAAGCCTCAAGAGGTGTGCGTGGCCGTGTGGCGC |
| | | AAGAACGACGAAAACATCACTCTGGAAACCGTGTGCCATGATCCTAAGCTC |
| | | CCGTACCATGACTTCATTCTTGAAGATGCAGCCTCCCCCAAGTGCATCATG |
| | | AAGGAGAAGAAGAAGCCTGGCGAAACCTTCTTCATGTGCTCATGCAGCTCC |
| | | GACGAATGTAACGACAACATCATCTTCTCAGAGGAATACAACACCTCAAAC |
| | | CCGGAC |
| SEQ ID | aCSF1R | ATGGAAACCGACACACTGCTGCTGTGGGTGCTGCTCTTGTGGGTGCCAGGA |
| NO: | BI123 VH- | TCTACAGGACAGGTGCAGCTTGTGCAAAGCGGGGGAGGCGTGGTGCAGCCG |
| 318 | 3 x 4GS- | GGACGGAGCCTGCGGCTGTCGTGCGCCGCGAGCGGTTTCACCTTCTCCTCC |
| | aCSF1R | TACGCTATGCACTGGGTCCGCCAGGCGCCGGGCAAGGGCCTCGAATGGGTG |
| | BI123 VL- | GCCGTGATCAGCTACGATGGCTCCAACAAGTACTACGCCGACTCAGTGAAG |
| | 4GS- | GGAAGGTTCACCATCTCAAGAGATAACAGCAAGAACACGCTGTACCTCCAG |
| | hFc_Hole- | ATGAACTCCCTCCGGGCCGAAGACACAGCCGTCTACTACTGTGCGAGAGAG |
| | 3 x 4GS- | CTCGATTACGGCGATTACGGCCTGGACGTGTGGGGCAAGGGAACCACGGTG |
| | TGFbR2 | ACCGTCTCCTCCGGTGGCGGAGGAAGCGGAGGCGGAGGTTCTGGCGGCGGA |
| | | GGATCTTCCCTCGTGCTGACCCAACCTCCTTCAGTCTCCGTGGCCCCCGGC |
| | | AAGACCGCCCGCATCACCTGTGGCGGTAACAACATCGGCTCCAAGTCAGTC |
| | | CACTGGTACCAGCAGAAGCCGGGCCAAGCTCCCGTGCTGGTCGTGTACGAC |
| | | GACTCAGACCGCCCCAGCGGTATCCCTGAGAGATTTAGCGGATCCAACTCC |
| | | GGGAACACCGCTACCCTGACCATCTCAAGGGTGGAGGCCGGAGACGAAGCC |
| | | GACTACTACTGCCAGGTGTGGGATTCCAGCTCCGACCACGTGGTGTTCGGC |
| | | GGCGGCACTCAGCTCACCGTGCTGGGTGGCGGAGGAAGCGATAAGACCCAC |
| | | ACCTGTCCTCCCTGCCCTGCCCCTGAACTGCTGGGCGGACCTAGCGTGTTC |
| | | CTGTTCCCTCCAAAGCCCAAGGACACCCTGATGATCAGCCGGACCCCTGAA |
| | | GTGACCTGCGTGGTGGTGGATGTGTCCCACGAGGATCCCGAAGTGAAGTTC |
| | | AATTGGTACGTGGACGGCGTGGAAGTGCACAACGCCAAGACCAAGCCCAGA |
| | | GAGGAACAGTACAACAGCACCTACCGGGTGGTGTCCGTGCTGACCGTGCTG |
| | | CACCAGGACTGGCTGAACGGCAAAGAGTACAAGTGCAAGGTGTCCAACAAG |
| | | GCCCTGCCAGCCCCTATCGAGAAAACCATCAGCAAGGCCAAGGGCCAGCCT |
| | | AGAGAGCCTCAGGTCTGCACCCTGCCTCCCAGCCGGGAAGAGATGACCAAG |
| | | AACCAGGTGTCCCTGAGCTGCGCCGTGAAGGGCTTCTACCCCTCCGATATC |
| | | GCCGTGGAATGGGAGAGCAACGGCCAGCCCGAGAACAACTACAAGACCACC |
| | | CCTCCCGTGCTGGACAGCGACGGCAGCTTCTTCCTGGTGTCCAAACTGACC |
| | | GTGGACAAGAGCCGGTGGCAGCAGGGCAATGTGTTCAGCTGTAGCGTGATG |
| | | CACGAGGCCCTGCACAACCACTACACCCAGAAGTCTCTGAGCCTGAGCCCT |
| | | GGCAAAGGTGGCGGAGGAAGCGGAGGCGGAGGTTCTGGCGGCGGAGGATCT |
| | | ATTCCTCCACACGTGCAAAAGTCGGTGAACAATGATATGATCGTCACCGAC |
| | | AACAACGGAGCCGTGAAGTTTCCTCAGCTGTGCAAGTTTTGTGATGTCCGC |
| | | TTCAGCACCTGTGACAACCAGAAGTCATGCATGTCGAACTGTTCGATCACC |
| | | AGCATTTGCGAAAAGCCTCAAGAGGTGTGCGTGGCCGTGTGGCGCAAGAAC |
| | | GACGAAAACATCACTCTGGAAACCGTGTGCCATGATCCTAAGCTCCCGTAC |
| | | CATGACTTCATTCTTGAAGATGCAGCCTCCCCCAAGTGCATCATGAAGGAG |
| | | AAGAAGAAGCCTGGCGAAACCTTCTTCATGTGCTCATGCAGCTCCGACGAA |
| | | TGTAACGACAACATCATCTTCTCAGAGGAATACAACACCTCAAACCCGGAC |
| SEQ ID | TGFbR2- | ATGGGCCGCGGACTTCTGCGCGGGCTGTGGCCTCTGCACATCGTGCTGTGG |
| NO: | 3 x 4GS- | ACCCGCATCGCGTCCACTATTCCTCCACACGTGCAAAAGTCGGTGAACAAT |
| 319 | hCH1- | GATATGATCGTCACCGACAACAACGGAGCCGTGAAGTTTCCTCAGCTGTGC |
| | hFc_Hole | AAGTTTTGTGATGTCCGCTTCAGCACCTGTGACAACCAGAAGTCATGCATG |
| | | TCGAACTGTTCGATCACCAGCATTTGCGAAAAGCCTCAAGAGGTGTGCGTG |
| | | GCCGTGTGGCGCAAGAACGACGAAAACATCACTCTGGAAACCGTGTGCCAT |
| | | GATCCTAAGCTCCCGTACCATGACTTCATTCTTGAAGATGCAGCCTCCCCC |
| | | AAGTGCATCATGAAGGAGAAGAAGAAGCCTGGCGAAACCTTCTTCATGTGC |
| | | TCATGCAGCTCCGACGAATGTAACGACAACATCATCTTCTCAGAGGAATAC |
| | | AACACCTCAAACCCGGACGGTGGCGGAGGAAGCGGAGGCGGAGGTTCTGGC |
| | | GGCGGAGGATCTGCTTCTACCAAGGGACCCAGCGTGTTCCCTCTGGCTCCT |
| | | TCCAGCAAGTCTACCTCTGGCGGAACAGCTGCTCTGGGCTGCCTGGTCAAG |
| | | GACTACTTTCCTGAGCCTGTGACCGTGTCTTGGAACTCTGGCGCTCTGACA |
| | | TCCGGCGTGCACACATTTCCAGCTGTGCTGCAGTCCTCCGGCCTGTACTCT |
| | | CTGTCCTCTGTCGTGACCGTGCCTTCCAGCTCTCTGGGAACCCAGACCTAC |
| | | ATCTGCAATGTGAACCACAAGCCTTCCAACACCAAGGTGGACAAGAGAGTG |
| | | GAACCCAAGTCCTGCGATAAGACCCACACCTGTCCTCCCTGCCCTGCCCCT |
| | | GAACTGCTGGGCGGACCTAGCGTGTTCCTGTTCCCTCCAAAGCCCAAGGAC |
| | | ACCCTGATGATCAGCCGGACCCCTGAAGTGACCTGCGTGGTGGTGGATGTG |
| | | TCCCACGAGGATCCCGAAGTGAAGTTCAATTGGTACGTGGACGGCGTGGAA |
| | | GTGCACAACGCCAAGACCAAGCCCAGAGAGGAACAGTACAACAGCACCTAC |
| | | CGGGTGGTGTCCGTGCTGACCGTGCTGCACCAGGACTGGCTGAACGGCAAA |
| | | GAGTACAAGTGCAAGGTGTCCAACAAGGCCCTGCCAGCCCCTATCGAGAAA |
| | | ACCATCAGCAAGGCCAAGGGCCAGCCTAGAGAGCCTCAGGTCTGCACCCTG |
| | | CCTCCCAGCCGGGAAGAGATGACCAAGAACCAGGTGTCCCTGAGCTGCGCC |
| | | GTGAAGGGCTTCTACCCCTCCGATATCGCCGTGGAATGGGAGAGCAACGGC |
| | | CAGCCCGAGAACAACTACAAGACCACCCCTCCCGTGCTGGACAGCGACGGC |
| | | AGCTTCTTCCTGGTGTCCAAACTGACCGTGGACAAGAGCCGGTGGCAGCAG |
| | | GGCAATGTGTTCAGCTGTAGCGTGATGCACGAGGCCCTGCACAACCACTAC |
| | | ACCCAGAAGTCTCTGAGCCTGAGCCCTGGCAAA |
| SEQ ID | TGFbR2- | ATGGGCCGCGGACTTCTGCGCGGGCTGTGGCCTCTGCACATCGTGCTGTGG |
| NO: | 3 x 4GS- | ACCCGCATCGCGTCCACTATTCCTCCACACGTGCAAAAGTCGGTGAACAAT |
| 320 | hCLIg_v1 | GATATGATCGTCACCGACAACAACGGAGCCGTGAAGTTTCCTCAGCTGTGC |
| | | AAGTTTTGTGATGTCCGCTTCAGCACCTGTGACAACCAGAAGTCATGCATG |
| | | TCGAACTGTTCGATCACCAGCATTTGCGAAAAGCCTCAAGAGGTGTGCGTG |
| | | GCCGTGTGGCGCAAGAACGACGAAAACATCACTCTGGAAACCGTGTGCCAT |
| | | GATCCTAAGCTCCCGTACCATGACTTCATTCTTGAAGATGCAGCCTCCCCC |
| | | AAGTGCATCATGAAGGAGAAGAAGAAGCCTGGCGAAACCTTCTTCATGTGC |
| | | TCATGCAGCTCCGACGAATGTAACGACAACATCATCTTCTCAGAGGAATAC |
| | | AACACCTCAAACCCGGACGGTGGCGGAGGAAGCGGAGGCGGAGGTTCTGGC |
| | | GGCGGAGGATCTGGACAACCTAAGGCCAATCCTACCGTGACACTGTTCCCT |
| | | CCATCCTCCGAGGAACTGCAGGCCAACAAGGCTACCCTCGTGTGCCTGATC |
| | | TCCGACTTTTACCCTGGCGCTGTGACCGTGGCCTGGAAGGCTGATGGATCT |
| | | CCTGTGAAGGCTGGCGTGGAAACCACCAAGCCTTCCAAGCAGTCCAACAAC |
| | | AAATACGCCGCCTCCTCCTACCTGTCTCTGACCCCTGAACAGTGGAAGTCC |
| | | CACCGGTCCTACAGCTGCCAAGTGACCCATGAGGGCTCCACCGTGGAAAAG |
| | | ACCGTGGCTCCTACCGAGTGCTCC |
| SEQ ID | aCCR2 | ATGGAAACCGACACACTGCTGCTGTGGGTGCTGCTCTTGTGGGTGCCAGGA |
| NO: | Hum1 VH- | TCTACAGGAGAAGTGCAGCTGGTGGAAAGCGGTGGCGGTCTGGTGCAGCCT |
| 321 | hCH1- | GGCGGTTCCCTCCGGCTGTCATGTGCTGCCTCCGGATTCTCGTTCAACGCG |
| | hFc_Knob- | TATGCCATGAACTGGGTGAGGCAGGCCCCGGGAAAGGGACTGGAATGGGTG |
| | 3 x 4GS- | GGGAGAATCCGGACCAAGAACAATAACTACGCTACTTACTACGCCGACTCC |
| | BI117 VH- | GTCAAGGACCGGTTCACAATTTCCCGGGACGACAGCAAGAGCTCACTGTAC |
| | 3 x 4GS- | CTTCAGATGAACAGCCTCAAAACCGAGGACACCGCCGTGTACTACTGTGCC |
| | aCSF1R | ACCTTTTACGGCAACGGTGTGTGGGGACAGGGAACAACTGTCACCGTGTCC |
| | BI117 VL | TCCGCTTCTACCAAGGGACCCAGCGTGTTCCCTCTGGCTCCTTCCAGCAAG |
| | | TCTACCTCTGGCGGAACAGCTGCTCTGGGCTGCCTGGTCAAGGACTACTTT |
| | | CCTGAGCCTGTGACCGTGTCTTGGAACTCTGGCGCTCTGACATCCGGCGTG |
| | | CACACATTTCCAGCTGTGCTGCAGTCCTCCGGCCTGTACTCTCTGTCCTCT |
| | | GTCGTGACCGTGCCTTCCAGCTCTCTGGGAACCCAGACCTACATCTGCAAT |
| | | GTGAACCACAAGCCTTCCAACACCAAGGTGGACAAGAGAGTGGAACCCAAG |
| | | TCCTGCGATAAGACCCACACATGTCCTCCATGCCCTGCCCCTGAGCTGCTG |
| | | GGCGGACCTTCCGTGTTCCTGTTCCCTCCAAAGCCCAAGGACACCCTGATG |
| | | ATCAGCCGGACCCCTGAAGTGACCTGCGTGGTGGTGGATGTGTCCCACGAG |
| | | GATCCCGAAGTGAAGTTCAATTGGTACGTGGACGGCGTGGAAGTGCACAAC |
| | | GCCAAGACCAAGCCCAGAGAGGAACAGTACAACAGCACCTACCGGGTGGTG |
| | | TCCGTGCTGACCGTGCTGCATCAGGACTGGCTGAACGGCAAAGAGTACAAG |
| | | TGCAAGGTGTCCAACAAGGCCCTGCCTGCCCCTATCGAGAAAACCATCAGC |
| | | AAGGCCAAGGGCCAGCCCCGCGAACCTCAGGTGTACACACTGCCTCCCTGC |
| | | CGGGAAGAGATGACCAAGAACCAGGTGTCCCTGTGGTGCCTGGTCAAGGGC |
| | | TTCTACCCCTCCGATATCGCCGTGGAATGGGAGAGCAACGGCCAGCCCGAG |
| | | AACAACTACAAGACCACCCCTCCCGTGCTGGACAGCGACGGCAGCTTCTTC |
| | | CTGTACTCCAAACTGACCGTGGACAAGAGCCGGTGGCAGCAGGGCAATGTG |
| | | TTCAGCTGTAGCGTGATGCACGAGGCCCTGCACAACCACTACACCCAGAAG |
| | | TCCCTGAGCCTGTCTCCTGGCAAAGGTGGCGGAGGAAGCGGAGGCGGAGGT |
| | | TCTGGCGGCGGAGGATCTGAAGTGCAGCTGGTCGAAAGCGGAGGGGGTCTG |
| | | GTCCAGCCTGGAGGGTCCCTGCGGCTGTCCTGCGCCGCTTCCGGCTTCACC |
| | | TTCTCCAACGATGAAATGAACTGGATCCGCCAAGCCCCTGGAAAGGGCCTG |
| | | GAATGGGTGTCGTACATCTCGAGCAGGTCCAGCACTATCTACTATGCCGAC |
| | | GCGGTGAAGGGACGCTTCACTATCAGCCGCGATAACGCACGCAATTCCCTC |
| | | TACCTGCAGATGAACTCGCTCAGAGCTGAGGATACTGCCGTCTACTACTGC |
| | | GCCCGCGGCGTGGACGGCGATTTTTCCTTCGATTACTGGGGACAGGGCACT |
| | | CTTGTCACCGTGTCAAGCGGTGGCGGAGGAAGCGGAGGCGGAGGTTCTGGC |
| | | GGCGGAGGATCTCAACTGGTGCTGACCCAGCCCCCTTCCGTCTCCGGCGCC |
| | | CCGGGCCAGCGCGTCACTATCTCCTGTACCGGCAGCTCCAGCAACATCGGA |
| | | GCAGGATACGACGTGCACTGGTACCAACAGCTCCCCGGAACCGCGCCTAAG |
| | | CTGCTGATCTACGGGAACTCTAACCGGCCGTCCGGCGTGCCTGACCGCTTC |
| | | AGCGGATCGAAGTCCGGCACCAGCGCCTCCCTGGCCATCACTGGACTGCAG |
| | | GCCGAGGACGAAGCCGATTACTACTGCCAGTCCTACGACTCCTCCCTGTCC |
| | | GAGGAAGTGTTCGGAGGCGGGACGAAGCTCACCGTCCTG |
| SEQ ID | aCCR2 | ATGGAAACCGACACACTGCTGCTGTGGGTGCTGCTCTTGTGGGTGCCAGGA |
| NO: | Hum1 VH- | TCTACAGGAGAAGTGCAGCTGGTGGAAAGCGGTGGCGGTCTGGTGCAGCCT |
| 322 | hCH1- | GGCGGTTCCCTCCGGCTGTCATGTGCTGCCTCCGGATTCTCGTTCAACGCG |
| | hFc_Knob- | TATGCCATGAACTGGGTGAGGCAGGCCCCGGGAAAGGGACTGGAATGGGTG |
| | 3 x 4GS- | GGGAGAATCCGGACCAAGAACAATAACTACGCTACTTACTACGCCGACTCC |
| | BI123 VH- | GTCAAGGACCGGTTCACAATTTCCCGGGACGACAGCAAGAGCTCACTGTAC |
| | 3 x 4GS- | CTTCAGATGAACAGCCTCAAAACCGAGGACACCGCCGTGTACTACTGTGCC |
| | aCSF1R | ACCTTTTACGGCAACGGTGTGTGGGGACAGGGAACAACTGTCACCGTGTCC |
| | BI123 VL | TCCGCTTCTACCAAGGGACCCAGCGTGTTCCCTCTGGCTCCTTCCAGCAAG |
| | | TCTACCTCTGGCGGAACAGCTGCTCTGGGCTGCCTGGTCAAGGACTACTTT |
| | | CCTGAGCCTGTGACCGTGTCTTGGAACTCTGGCGCTCTGACATCCGGCGTG |
| | | CACACATTTCCAGCTGTGCTGCAGTCCTCCGGCCTGTACTCTCTGTCCTCT |
| | | GTCGTGACCGTGCCTTCCAGCTCTCTGGGAACCCAGACCTACATCTGCAAT |
| | | GTGAACCACAAGCCTTCCAACACCAAGGTGGACAAGAGAGTGGAACCCAAG |
| | | TCCTGCGATAAGACCCACACATGTCCTCCATGCCCTGCCCCTGAGCTGCTG |
| | | GGCGGACCTTCCGTGTTCCTGTTCCCTCCAAAGCCCAAGGACACCCTGATG |
| | | ATCAGCCGGACCCCTGAAGTGACCTGCGTGGTGGTGGATGTGTCCCACGAG |
| | | GATCCCGAAGTGAAGTTCAATTGGTACGTGGACGGCGTGGAAGTGCACAAC |
| | | GCCAAGACCAAGCCCAGAGAGGAACAGTACAACAGCACCTACCGGGTGGTG |
| | | TCCGTGCTGACCGTGCTGCATCAGGACTGGCTGAACGGCAAAGAGTACAAG |
| | | TGCAAGGTGTCCAACAAGGCCCTGCCTGCCCCTATCGAGAAAACCATCAGC |
| | | AAGGCCAAGGGCCAGCCCCGCGAACCTCAGGTGTACACACTGCCTCCCTGC |
| | | CGGGAAGAGATGACCAAGAACCAGGTGTCCCTGTGGTGCCTGGTCAAGGGC |
| | | TTCTACCCCTCCGATATCGCCGTGGAATGGGAGAGCAACGGCCAGCCCGAG |
| | | AACAACTACAAGACCACCCCTCCCGTGCTGGACAGCGACGGCAGCTTCTTC |
| | | CTGTACTCCAAACTGACCGTGGACAAGAGCCGGTGGCAGCAGGGCAATGTG |
| | | TTCAGCTGTAGCGTGATGCACGAGGCCCTGCACAACCACTACACCCAGAAG |
| | | TCCCTGAGCCTGTCTCCTGGCAAAGGTGGCGGAGGAAGCGGAGGCGGAGGT |
| | | TCTGGCGGCGGAGGATCTCAGGTGCAGCTTGTGCAAAGCGGGGGAGGCGTG |
| | | GTGCAGCCGGGACGGAGCCTGCGGCTGTCGTGCGCCGCGAGCGGTTTCACC |
| | | TTCTCCTCCTACGCTATGCACTGGGTCCGCCAGGCGCCGGGCAAGGGCCTC |
| | | GAATGGGTGGCCGTGATCAGCTACGATGGCTCCAACAAGTACTACGCCGAC |
| | | TCAGTGAAGGGAAGGTTCACCATCTCAAGAGATAACAGCAAGAACACGCTG |
| | | TACCTCCAGATGAACTCCCTCCGGGCCGAAGACACAGCCGTCTACTACTGT |
| | | GCGAGAGAGCTCGATTACGGCGATTACGGCCTGGACGTGTGGGGCAAGGGA |
| | | ACCACGGTGACCGTCTCCTCCGGTGGCGGAGGAAGCGGAGGCGGAGGTTCT |
| | | GGCGGCGGAGGATCTTCCCTCGTGCTGACCCAACCTCCTTCAGTCTCCGTG |
| | | GCCCCCGGCAAGACCGCCCGCATCACCTGTGGCGGTAACAACATCGGCTCC |
| | | AAGTCAGTCCACTGGTACCAGCAGAAGCCGGGCCAAGCTCCCGTGCTGGTC |
| | | GTGTACGACGACTCAGACCGCCCCAGCGGTATCCCTGAGAGATTTAGCGGA |
| | | TCCAACTCCGGGAACACCGCTACCCTGACCATCTCAAGGGTGGAGGCCGGA |
| | | GACGAAGCCGACTACTACTGCCAGGTGTGGGATTCCAGCTCCGACCACGTG |
| | | GTGTTCGGCGGCGGCACTCAGCTCACCGTGCTG |
| SEQ ID | hFc_Knob | ATGGAAACCGACACACTGCTGCTGTGGGTGCTGCTCTTGTGGGTGCCAGGA |
| NO: | (H435R, | TCTACAGGAGACAAGACCCATACCTGTCCTCCATGTCCTGCTCCAGAGCTG |
| 397 | Y436F) | CTCGGAGGCCCTTCCGTGTTTCTGTTCCCTCCAAAGCCTAAGGACACCCTG |
| | | ATGATCTCTCGGACCCCTGAAGTGACCTGCGTGGTGGTGGATGTGTCTCAC |
| | | GAGGATCCCGAAGTGAAGTTCAATTGGTACGTGGACGGCGTGGAAGTGCAC |
| | | AACGCCAAGACCAAGCCTAGAGAGGAACAGTACAACTCCACCTACAGAGTG |
| | | GTGTCCGTGCTGACCGTGCTGCACCAGGATTGGCTGAACGGCAAAGAGTAC |
| | | AAGTGCAAGGTGTCCAACAAGGCCCTGCCTGCTCCTATCGAAAAGACCATC |
| | | TCCAAGGCCAAGGGCCAGCCTAGGGAACCCCAGGTTTACACCCTGCCTCCA |
| | | TGCCGGGAAGAGATGACCAAGAACCAGGTGTCCCTGTGGTGCCTGGTCAAG |
| | | GGCTTCTACCCTTCCGATATCGCCGTGGAATGGGAGAGCAATGGCCAGCCT |
| | | GAGAACAACTACAAGACCACACCTCCTGTGCTGGACTCCGACGGCTCATTC |
| | | TTCCTGTACTCCAAGCTGACAGTGGACAAGTCCAGATGGCAGCAGGGCAAC |
| | | GTGTTCTCCTGCTCCGTGATGCACGAGGCCCTGCACAACAGATTCACCCAG |
| | | AAGTCCCTGTCTCTGTCCCCTGGAAAAGGCGGCGGAGGATCTGGCGGAGGC |
| | | GGATCTCACCATCACCATCATCACCACCAC |
| SEQ ID | hTGFbR2 | ATGGGCCGCGGACTTCTGCGCGGGCTGTGGCCTCTGCACATCGTGCTGTGG |
| NO: | signal | ACCCGCATCGCGTCCACT |
| 275 | peptide | |
| SEQ ID | hTGFbR2 | ATTCCTCCACACGTGCAAAAGTCGGTGAACAATGATATGATCGTCACCGAC |
| NO: | | AACAACGGAGCCGTGAAGTTTCCTCAGCTGTGCAAGTTTTGTGATGTCCGC |
| 276 | | TTCAGCACCTGTGACAACCAGAAGTCATGCATGTCGAACTGTTCGATCACC |
| | | AGCATTTGCGAAAAGCCTCAAGAGGTGTGCGTGGCCGTGTGGCGCAAGAAC |
| | | GACGAAAACATCACTCTGGAAACCGTGTGCCATGATCCTAAGCTCCCGTAC |
| | | CATGACTTCATTCTTGAAGATGCAGCCTCCCCCAAGTGCATCATGAAGGAG |
| | | AAGAAGAAGCCTGGCGAAACCTTCTTCATGTGCTCATGCAGCTCCGACGAA |
| | | TGTAACGACAACATCATCTTCTCAGAGGAATACAACACCTCAAACCCGGAC |
| SEQ ID | IL2 | GCCCCTACCAGCAGCAGCACCAAGAAAACCCAGCTCCAGCTCGAGCACCTC |
| NO: | | CTGCTGGACCTGCAGATGATCCTGAACGGCATCAACAACTACAAGAACCCC |
| 277 | | AAGCTGACCCGGATGCTGACCTTCAAGTTCTACATGCCCAAGAAGGCCACC |
| | | GAGCTGAAGCACCTCCAGTGCCTGGAAGAGGAACTGAAGCCCCTGGAAGAA |
| | | GTGCTGAACCTGGCCCAGAGCAAGAACTTCCACCTGAGGCCCAGGGACCTG |
| | | ATCAGCAACATCAACGTGATCGTGCTGGAACTGAAAGGCAGCGAGACAACC |
| | | TTCATGTGCGAGTACGCCGACGAGACAGCCACCATCGTGGAATTTCTGAAC |
| | | CGGTGGATCACCTTCTGCCAGAGCATCATCAGCACCCTGACA |
| SEQ ID | 3 × 4GS | GGTGGCGGAGGAAGCGGAGGCGGAGGTTCTGGCGGCGGAGGATCT |
| NO: | linker | |
| 278 | | |
| SEQ ID | 4GS linker | GGTGGCGGAGGAAGC |
| NO: | | |
| 279 | | |
| SEQ ID | 2 × 4GS | GGCGGCGGAGGATCTGGCGGAGGCGGATCT |
| NO: | linker | |
| 394 | | |
| SEQ ID | hFc_Knob | GACAAGACCCATACCTGTCCTCCATGTCCTGCTCCAGAGCTGCTCGGAGGC |
| NO: | (H435R, | CCTTCCGTGTTTCTGTTCCCTCCAAAGCCTAAGGACACCCTGATGATCTCT |
| 395 | Y436F) | CGGACCCCTGAAGTGACCTGCGTGGTGGTGGATGTGTCTCACGAGGATCCC |
| | | GAAGTGAAGTTCAATTGGTACGTGGACGGCGTGGAAGTGCACAACGCCAAG |
| | | ACCAAGCCTAGAGAGGAACAGTACAACTCCACCTACAGAGTGGTGTCCGTG |
| | | CTGACCGTGCTGCACCAGGATTGGCTGAACGGCAAAGAGTACAAGTGCAAG |
| | | GTGTCCAACAAGGCCCTGCCTGCTCCTATCGAAAAGACCATCTCCAAGGCC |
| | | AAGGGCCAGCCTAGGGAACCCCAGGTTTACACCCTGCCTCCATGCCGGGAA |
| | | GAGATGACCAAGAACCAGGTGTCCCTGTGGTGCCTGGTCAAGGGCTTCTAC |
| | | CCTTCCGATATCGCCGTGGAATGGGAGAGCAATGGCCAGCCTGAGAACAAC |
| | | TACAAGACCACACCTCCTGTGCTGGACTCCGACGGCTCATTCTTCCTGTAC |
| | | TCCAAGCTGACAGTGGACAAGTCCAGATGGCAGCAGGGCAACGTGTTCTCC |
| | | TGCTCCGTGATGCACGAGGCCCTGCACAACAGATTCACCCAGAAGTCCCTG |
| | | TCTCTGTCCCCTGGAAAA |
| SEQ ID | 8 × His | CACCATCACCATCATCACCACCAC |
| NO: | | |
| 396 | | |

TABLE 26
Components of full-length heavy chain and light chain sequences.

| Full | | | | | | | | | | |
|---|---|---|---|---|---|---|---|---|---|---|
| length | Variable | Linker | Variable | Linker | Constant | Fc | Linker | Variable | Linker | Variable |
| SEQ ID | SEQ ID | | | | SEQ ID | SEQ ID | | | | |
| NO: 71 | NO: 44 | | | | NO: 46 | NO: 52 | | | | |
| SEQ ID | SEQ ID | | | | SEQ ID | | | | | |
| NO: 72 | NO: 45 | | | | NO: 47 | | | | | |
| SEQ ID | SEQ ID | | | | SEQ ID | SEQ ID | | | | |
| NO: 73 | NO: 48 | | | | NO: 49 | NO: 53 | | | | |
| SEQ ID | SEQ ID | | | | SEQ ID | | | | | |
| NO: 74 | NO: 50 | | | | NO: 51 | | | | | |
| SEQ ID | SEQ ID | | | | SEQ ID | SEQ ID | | | | |
| NO: 75 | NO: 54 | | | | NO: 55 | NO: 56 | | | | |
| SEQ ID | SEQ ID | | | | SEQ ID | | | | | |
| NO: 76 | NO: 57 | | | | NO: 58 | | | | | |
| SEQ ID | SEQ ID | | | | SEQ ID | SEQ ID | | | | |
| NO: 77 | NO: 59 | | | | NO: 55 | NO: 56 | | | | |
| SEQ ID | SEQ ID | | | | SEQ ID | | | | | |
| NO: 78 | NO: 60 | | | | NO: 61 | | | | | |
| SEQ ID | SEQ ID | | | | SEQ ID | SEQ ID | | | | |
| NO: 79 | NO: 62 | | | | NO: 55 | NO: 56 | | | | |
| SEQ ID | SEQ ID | | | | SEQ ID | | | | | |
| NO: 80 | NO: 63 | | | | NO: 58 | | | | | |
| SEQ ID | SEQ ID | | | | SEQ ID | SEQ ID | | | | |
| NO: 81 | NO: 64 | | | | NO: 55 | NO: 56 | | | | |
| SEQ ID | SEQ ID | | | | SEQ ID | | | | | |
| NO: 82 | NO: 65 | | | | NO: 58 | | | | | |
| SEQ ID | SEQ ID | | | | SEQ ID | SEQ ID | | | | |
| NO: 83 | NO: 66 | | | | NO: 55 | NO: 68 | | | | |
| SEQ ID | SEQ ID | | | | SEQ ID | | | | | |
| NO: 84 | NO: 67 | | | | NO: 58 | | | | | |
| SEQ ID | SEQ ID | | | | SEQ ID | SEQ ID | | | | |
| NO: 85 | NO: 69 | | | | NO: 55 | NO: 68 | | | | |
| SEQ ID | SEQ ID | | | | SEQ ID | | | | | |
| NO: 86 | NO: 70 | | | | NO: 58 | | | | | |
| SEQ ID | SEQ ID | | | | SEQ ID | SEQ ID | | | | |
| NO: 351 | NO: 323 | | | | NO: 55 | NO: 68 | | | | |
| SEQ ID | SEQ ID | | | | SEQ ID | SEQ ID | | | | |
| NO: 352 | NO: 324 | | | | NO: 55 | NO: 68 | | | | |
| SEQ ID | SEQ ID | | | | SEQ ID | SEQ ID | | | | |
| NO: 353 | NO: 325 | | | | NO: 55 | NO: 68 | | | | |
| SEQ ID | SEQ ID | | | | SEQ ID | SEQ ID | | | | |
| NO: 354 | NO: 326 | | | | NO: 55 | NO: 68 | | | | |
| SEQ ID | SEQ ID | | | | SEQ ID | SEQ ID | | | | |
| NO: 355 | NO: 327 | | | | NO: 55 | NO: 68 | | | | |
| SEQ ID | SEQ ID | | | | SEQ ID | SEQ ID | | | | |
| NO: 356 | NO: 328 | | | | NO: 55 | NO: 68 | | | | |
| SEQ ID | SEQ ID | | | | SEQ ID | SEQ ID | | | | |
| NO: 357 | NO: 329 | | | | NO: 55 | NO: 68 | | | | |
| SEQ ID | SEQ ID | | | | SEQ ID | SEQ ID | | | | |
| NO: 358 | NO: 330 | | | | NO: 55 | NO: 68 | | | | |
| SEQ ID | SEQ ID | | | | SEQ ID | SEQ ID | | | | |
| NO: 359 | NO: 331 | | | | NO: 55 | NO: 68 | | | | |
| SEQ ID | SEQ ID | | | | SEQ ID | SEQ ID | | | | |
| NO: 360 | NO: 332 | | | | NO: 55 | NO: 68 | | | | |
| SEQ ID | SEQ ID | | | | SEQ ID | SEQ ID | | | | |
| NO: 361 | NO: 333 | | | | NO: 55 | NO: 68 | | | | |
| SEQ ID | SEQ ID | | | | SEQ ID | SEQ ID | | | | |
| NO: 362 | NO: 334 | | | | NO: 55 | NO: 68 | | | | |
| SEQ ID | SEQ ID | | | | SEQ ID | SEQ ID | | | | |
| NO: 363 | NO: 335 | | | | NO: 55 | NO: 68 | | | | |
| SEQ ID | SEQ ID | | | | SEQ ID | SEQ ID | | | | |
| NO: 364 | NO: 336 | | | | NO: 55 | NO: 68 | | | | |
| SEQ ID | SEQ ID | | | | SEQ ID | SEQ ID | | | | |
| NO: 365 | NO: 337 | | | | NO: 55 | NO: 68 | | | | |
| SEQ ID | SEQ ID | | | | SEQ ID | SEQ ID | | | | |
| NO: 366 | NO: 338 | | | | NO: 55 | NO: 68 | | | | |
| SEQ ID | SEQ ID | | | | SEQ ID | SEQ ID | | | | |
| NO: 367 | NO: 339 | | | | NO: 55 | NO: 68 | | | | |
| SEQ ID | SEQ ID | | | | SEQ ID | SEQ ID | | | | |
| NO: 368 | NO: 340 | | | | NO: 55 | NO: 68 | | | | |
| SEQ ID | SEQ ID | | | | SEQ ID | | | | | |
| NO: 369 | NO: 341 | | | | NO: 61 | | | | | |
| SEQ ID | SEQ ID | | | | SEQ ID | | | | | |
| NO: 370 | NO: 342 | | | | NO: 61 | | | | | |
| SEQ ID | SEQ ID | | | | SEQ ID | SEQ ID | | | | |
| NO: 371 | NO: 343 | | | | NO: 55 | NO: 56 | | | | |
| SEQ ID | SEQ ID | | | | SEQ ID | SEQ ID | | | | |
| NO: 372 | NO: 344 | | | | NO: 55 | NO: 56 | | | | |
| SEQ ID | SEQ ID | | | | SEQ ID | | | | | |
| NO: 373 | NO: 345 | | | | NO: 58 | | | | | |
| SEQ ID | SEQ ID | | | | SEQ ID | SEQ ID | SEQ ID | SEQ ID | SEQ ID | SEQ ID |
| NO: 374 | NO: 323 | | | | NO: 55 | NO: 68 | NO: 349 | NO: 346 | NO: 349 | NO: 347 |
| SEQ ID | SEQ ID | | | | SEQ ID | SEQ ID | SEQ ID | SEQ ID | SEQ ID | SEQ ID |
| NO: 375 | NO: 337 | | | | NO: 55 | NO: 68 | NO: 349 | NO: 346 | NO: 349 | NO: 347 |
| SEQ ID | SEQ ID | | | | SEQ ID | SEQ ID | SEQ ID | SEQ ID | SEQ ID | SEQ ID |
| NO: 376 | NO: 343 | | | | NO: 55 | NO: 56 | NO: 349 | NO: 346 | NO: 349 | NO: 347 |
| SEQ ID | SEQ ID | | | | SEQ ID | | SEQ ID | SEQ ID | | |
| NO: 377 | NO: 341 | | | | NO: 61 | | NO: 349 | NO: 246 | | |
| SEQ ID | SEQ ID | | | | SEQ ID | | | SEQ ID | | |
| NO: 378 | NO: 341 | | | | NO: 61 | | | NO: 246 | | |
| SEQ ID | SEQ ID | | | | SEQ ID | | SEQ ID | SEQ ID | | |
| NO: 379 | NO: 342 | | | | NO: 61 | | NO: 349 | NO: 246 | | |
| SEQ ID | SEQ ID | | | | SEQ ID | | | SEQ ID | | |
| NO: 380 | NO: 342 | | | | NO: 61 | | | NO: 246 | | |
| SEQ ID | SEQ ID | | | | SEQ ID | | SEQ ID | SEQ ID | | |
| NO: 381 | NO: 345 | | | | NO: 58 | | NO: 349 | NO: 246 | | |
| SEQ ID | SEQ ID | | | | SEQ ID | | | SEQ ID | | |
| NO: 382 | NO: 345 | | | | NO: 58 | | | NO: 246 | | |
| SEQ ID | SEQ ID | | | | SEQ ID | SEQ ID | SEQ ID | SEQ ID | | |
| NO: 383 | NO: 323 | | | | NO: 55 | NO: 68 | NO: 349 | NO: 101 | | |
| SEQ ID | SEQ ID | | | | SEQ ID | SEQ ID | SEQ ID | SEQ ID | | |
| NO: 384 | NO: 337 | | | | NO: 55 | NO: 68 | NO: 349 | NO: 101 | | |
| SEQ ID | SEQ ID | | | | SEQ ID | SEQ ID | SEQ ID | SEQ ID | | |
| NO: 385 | NO: 343 | | | | NO: 55 | NO: 56 | NO: 349 | NO: 101 | | |
| SEQ ID | SEQ ID | SEQ ID | SEQ ID | SEQ ID | | SEQ ID | | | | |
| NO: 386 | NO: 323 | NO: 349 | NO: 341 | NO: 350 | | NO: 68 | | | | |
| SEQ ID | SEQ ID | SEQ ID | SEQ ID | SEQ ID | | SEQ ID | | | | |
| NO: 387 | NO: 337 | NO: 349 | NO: 342 | NO: 350 | | NO: 68 | | | | |
| SEQ ID | SEQ ID | SEQ ID | SEQ ID | SEQ ID | | SEQ ID | SEQ ID | SEQ ID | | |
| NO: 388 | NO: 323 | NO: 349 | NO: 341 | NO: 350 | | NO: 68 | NO: 349 | NO: 101 | | |
| SEQ ID | SEQ ID | SEQ ID | SEQ ID | SEQ ID | | SEQ ID | SEQ ID | SEQ ID | | |
| NO: 389 | NO: 337 | NO: 349 | NO: 342 | NO: 350 | | NO: 68 | NO: 349 | NO: 101 | | |
| SEQ ID | SEQ ID | SEQ ID | | | SEQ ID | SEQ ID | | | | |
| NO: 390 | NO: 101 | NO: 349 | | | NO: 55 | NO: 68 | | | | |
| SEQ ID | SEQ ID | SEQ ID | | | SEQ ID | | | | | |
| NO: 391 | NO: 101 | NO: 349 | | | NO: 61 | | | | | |
| SEQ ID | SEQ ID | | | | SEQ ID | SEQ ID | SEQ ID | SEQ ID | SEQ ID | SEQ ID |
| NO: 392 | NO: 343 | | | | NO: 55 | NO: 56 | NO: 349 | NO: 323 | NO: 349 | NO: 341 |
| SEQ ID | SEQ ID | | | | SEQ ID | SEQ ID | SEQ ID | SEQ ID | SEQ ID | SEQ ID |
| NO: 393 | NO: 344 | | | | NO: 55 | NO: 56 | NO: 349 | NO: 337 | NO: 349 | NO: 342 |
| SEQ ID | | | | | | SEQ ID | SEQ ID | SEQ ID | | |
| NO: 401 | | | | | | NO: 399 | NO: 398 | NO: 400 | | |

TABLE 27
Sequences used to construct ORFs.

| Full | | | | | | | | | | |
|---|---|---|---|---|---|---|---|---|---|---|
| length | Variable | Linker | Variable | Linker | Constant | Fc | Linker | Variable | Linker | Variable |
| SEQ ID | SEQ ID | | | | SEQ ID | SEQ ID | | | | |
| NO: 28 | NO: 1 | | | | NO: 3 | NO: 9 | | | | |
| SEQ ID | SEQ ID | | | | SEQ ID | | | | | |
| NO: 29 | NO: 2 | | | | NO: 4 | | | | | |
| SEQ ID | SEQ ID | | | | SEQ ID | SEQ ID | | | | |
| NO: 30 | NO: 5 | | | | NO: 6 | NO: 10 | | | | |
| SEQ ID | SEQ ID | | | | SEQ ID | | | | | |
| NO: 31 | NO: 7 | | | | NO: 8 | | | | | |
| SEQ ID | SEQ ID | | | | SEQ ID | SEQ ID | | | | |
| NO: 32 | NO: 11 | | | | NO: 12 | NO: 13 | | | | |
| SEQ ID | SEQ ID | | | | SEQ ID | | | | | |
| NO: 33 | NO: 14 | | | | NO: 15 | | | | | |
| SEQ ID | SEQ ID | | | | SEQ ID | SEQ ID | | | | |
| NO: 34 | NO: 16 | | | | NO: 12 | NO: 13 | | | | |
| SEQ ID | SEQ ID | | | | SEQ ID | | | | | |
| NO: 35 | NO: 17 | | | | NO: 18 | | | | | |
| SEQ ID | SEQ ID | | | | SEQ ID | SEQ ID | | | | |
| NO: 36 | NO: 19 | | | | NO: 12 | NO: 13 | | | | |
| SEQ ID | SEQ ID | | | | SEQ ID | | | | | |
| NO: 37 | NO: 20 | | | | NO: 15 | | | | | |
| SEQ ID | SEQ ID | | | | SEQ ID | SEQ ID | | | | |
| NO: 38 | NO: 21 | | | | NO: 12 | NO: 13 | | | | |
| SEQ ID | SEQ ID | | | | SEQ ID | | | | | |
| NO: 39 | NO: 22 | | | | NO: 18 | | | | | |
| SEQ ID | SEQ ID | | | | SEQ ID | SEQ ID | | | | |
| NO: 40 | NO: 23 | | | | NO: 12 | NO: 25 | | | | |
| SEQ ID | SEQ ID | | | | SEQ ID | | | | | |
| NO: 41 | NO: 24 | | | | NO: 15 | | | | | |
| SEQ ID | SEQ ID | | | | SEQ ID | SEQ ID | | | | |
| NO: 42 | NO: 26 | | | | NO: 12 | NO: 25 | | | | |
| SEQ ID | SEQ ID | | | | SEQ ID | | | | | |
| NO: 43 | NO: 27 | | | | NO: 15 | | | | | |
| SEQ ID | SEQ ID | | | | SEQ ID | SEQ ID | | | | |
| NO: 280 | NO: 250 | | | | NO: 12 | NO: 25 | | | | |
| SEQ ID | SEQ ID | | | | SEQ ID | SEQ ID | | | | |
| NO: 281 | NO: 251 | | | | NO: 12 | NO: 25 | | | | |
| SEQ ID | SEQ ID | | | | SEQ ID | SEQ ID | | | | |
| NO: 282 | NO: 252 | | | | NO: 12 | NO: 25 | | | | |
| SEQ ID | SEQ ID | | | | SEQ ID | SEQ ID | | | | |
| NO: 283 | NO: 253 | | | | NO: 12 | NO: 25 | | | | |
| SEQ ID | SEQ ID | | | | SEQ ID | SEQ ID | | | | |
| NO: 284 | NO: 254 | | | | NO: 12 | NO: 25 | | | | |
| SEQ ID | SEQ ID | | | | SEQ ID | SEQ ID | | | | |
| NO: 285 | NO: 255 | | | | NO: 12 | NO: 25 | | | | |
| SEQ ID | SEQ ID | | | | SEQ ID | SEQ ID | | | | |
| NO: 286 | NO: 256 | | | | NO: 12 | NO: 25 | | | | |
| SEQ ID | SEQ ID | | | | SEQ ID | SEQ ID | | | | |
| NO: 287 | NO: 257 | | | | NO: 12 | NO: 25 | | | | |
| SEQ ID | SEQ ID | | | | SEQ ID | SEQ ID | | | | |
| NO: 288 | NO: 258 | | | | NO: 12 | NO: 25 | | | | |
| SEQ ID | SEQ ID | | | | SEQ ID | SEQ ID | | | | |
| NO: 289 | NO: 259 | | | | NO: 12 | NO: 25 | | | | |
| SEQ ID | SEQ ID | | | | SEQ ID | SEQ ID | | | | |
| NO: 290 | NO: 260 | | | | NO: 12 | NO: 25 | | | | |
| SEQ ID | SEQ ID | | | | SEQ ID | SEQ ID | | | | |
| NO: 291 | NO: 261 | | | | NO: 12 | NO: 25 | | | | |
| SEQ ID | SEQ ID | | | | SEQ ID | SEQ ID | | | | |
| NO: 292 | NO: 262 | | | | NO: 12 | NO: 25 | | | | |
| SEQ ID | SEQ ID | | | | SEQ ID | SEQ ID | | | | |
| NO: 293 | NO: 263 | | | | NO: 12 | NO: 25 | | | | |
| SEQ ID | SEQ ID | | | | SEQ ID | SEQ ID | | | | |
| NO: 294 | NO: 264 | | | | NO: 12 | NO: 25 | | | | |
| SEQ ID | SEQ ID | | | | SEQ ID | SEQ ID | | | | |
| NO: 295 | NO: 265 | | | | NO: 12 | NO: 25 | | | | |
| SEQ ID | SEQ ID | | | | SEQ ID | SEQ ID | | | | |
| NO: 296 | NO: 266 | | | | NO: 12 | NO: 25 | | | | |
| SEQ ID | SEQ ID | | | | SEQ ID | SEQ ID | | | | |
| NO: 297 | NO: 267 | | | | NO: 12 | NO: 25 | | | | |
| SEQ ID | SEQ ID | | | | SEQ ID | | | | | |
| NO: 298 | NO: 268 | | | | NO: 18 | | | | | |
| SEQ ID | SEQ ID | | | | SEQ ID | | | | | |
| NO: 299 | NO: 269 | | | | NO: 18 | | | | | |
| SEQ ID | SEQ ID | | | | SEQ ID | SEQ ID | | | | |
| NO: 300 | NO: 270 | | | | NO: 12 | NO: 13 | | | | |
| SEQ ID | SEQ ID | | | | SEQ ID | SEQ ID | | | | |
| NO: 301 | NO: 271 | | | | NO: 12 | NO: 13 | | | | |
| SEQ ID | SEQ ID | | | | SEQ ID | | | | | |
| NO: 302 | NO: 272 | | | | NO: 15 | | | | | |
| SEQ ID | SEQ ID | | | | SEQ ID | SEQ ID | SEQ ID | SEQ ID | SEQ ID | SEQ ID |
| NO: 303 | NO: 250 | | | | NO: 12 | NO: 25 | NO: 278 | NO: 273 | NO: 278 | NO: 274 |
| SEQ ID | SEQ ID | | | | SEQ ID | SEQ ID | SEQ ID | SEQ ID | SEQ ID | SEQ ID |
| NO: 304 | NO: 264 | | | | NO: 12 | NO: 25 | NO: 278 | NO: 273 | NO: 278 | NO: 274 |
| SEQ ID | SEQ ID | | | | SEQ ID | SEQ ID | SEQ ID | SEQ ID | SEQ ID | SEQ ID |
| NO: 305 | NO: 270 | | | | NO: 12 | NO: 13 | NO: 278 | NO: 273 | NO: 278 | NO: 274 |
| SEQ ID | SEQ ID | | | | SEQ ID | | SEQ ID | SEQ ID | | |
| NO: 306 | NO: 268 | | | | NO: 18 | | NO: 278 | NO: 277 | | |
| SEQ ID | SEQ ID | | | | SEQ ID | | | SEQ ID | | |
| NO: 307 | NO: 268 | | | | NO: 18 | | | NO: 277 | | |
| SEQ ID | SEQ ID | | | | SEQ ID | | SEQ ID | SEQ ID | | |
| NO: 308 | NO: 269 | | | | NO: 18 | | NO: 278 | NO: 277 | | |
| SEQ ID | SEQ ID | | | | SEQ ID | | | SEQ ID | | |
| NO: 309 | NO: 269 | | | | NO: 18 | | | NO: 277 | | |
| SEQ ID | SEQ ID | | | | SEQ ID | | SEQ ID | SEQ ID | | |
| NO: 310 | NO: 272 | | | | NO: 15 | | NO: 278 | NO: 277 | | |
| SEQ ID | SEQ ID | | | | SEQ ID | | | SEQ ID | | |
| NO: 311 | NO: 272 | | | | NO: 15 | | | NO: 277 | | |
| SEQ ID | SEQ ID | | | | SEQ ID | SEQ ID | SEQ ID | SEQ ID | | |
| NO: 312 | NO: 250 | | | | NO: 12 | NO: 25 | NO: 278 | NO: 276 | | |
| SEQ ID | SEQ ID | | | | SEQ ID | SEQ ID | SEQ ID | SEQ ID | | |
| NO: 313 | NO: 264 | | | | NO: 12 | NO: 25 | NO: 278 | NO: 276 | | |
| SEQ ID | SEQ ID | | | | SEQ ID | SEQ ID | SEQ ID | SEQ ID | | |
| NO: 314 | NO: 270 | | | | NO: 12 | NO: 13 | NO: 278 | NO: 276 | | |
| SEQ ID | SEQ ID | SEQ ID | SEQ ID | SEQ ID | | SEQ ID | | | | |
| NO: 315 | NO: 250 | NO: 278 | NO: 268 | NO: 279 | | NO: 25 | | | | |
| SEQ ID | SEQ ID | SEQ ID | SEQ ID | SEQ ID | | SEQ ID | | | | |
| NO: 316 | NO: 264 | NO: 278 | NO: 269 | NO: 279 | | NO: 25 | | | | |
| SEQ ID | SEQ ID | SEQ ID | SEQ ID | SEQ ID | | SEQ ID | SEQ ID | SEQ ID | | |
| NO: 317 | NO: 250 | NO: 278 | NO: 268 | NO: 279 | | NO: 25 | NO: 278 | NO: 276 | | |
| SEQ ID | SEQ ID | SEQ ID | SEQ ID | SEQ ID | | SEQ ID | SEQ ID | SEQ ID | | |
| NO: 318 | NO: 264 | NO: 278 | NO: 269 | NO: 279 | | NO: 25 | NO: 278 | NO: 276 | | |
| SEQ ID | SEQ ID | SEQ ID | | | SEQ ID | SEQ ID | | | | |
| NO: 319 | NO: 276 | NO: 278 | | | NO: 12 | NO: 25 | | | | |
| SEQ ID | SEQ ID | SEQ ID | | | SEQ ID | | | | | |
| NO: 320 | NO: 276 | NO: 278 | | | NO: 18 | | | | | |
| SEQ ID | SEQ ID | | | | SEQ ID | SEQ ID | SEQ ID | SEQ ID | SEQ ID | SEQ ID |
| NO: 321 | NO: 270 | | | | NO: 12 | NO: 13 | NO: 278 | NO: 250 | NO: 278 | NO: 268 |
| SEQ ID | SEQ ID | | | | SEQ ID | SEQ ID | SEQ ID | SEQ ID | SEQ ID | SEQ ID |
| NO: 322 | NO: 270 | | | | NO: 12 | NO: 13 | NO: 278 | NO: 264 | NO: 278 | NO: 269 |
| SEQ ID | | | | | | SEQ ID | SEQ ID | SEQ ID | | |
| NO: 397 | | | | | | NO: 395 | NO: 394 | NO: 396 | | |

TABLE 28
Exemplary multispecific antibody molecules.

| | | | SEQ ID | | SEQ ID | | SEQ ID | | SEQ ID |
|---|---|---|---|---|---|---|---|---|---|
| Multispecific | | | NO of | | NO of | | NO of | | NO of |
| Molecule | BIM ID | HC 1 | HC 1 | LC 1 | LC 1 | HC 2 | HC 2 | LC 2 | LC 2 |
| 10 | BIM0204 | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI117 | NO: 351 | BI117 | NO: 369 |
| | | hCH1- | | hCLIg_vk | | VH-hCH1- | | VL- | |
| | | hFc_Knob | | | | hFc_Hole | | hCLIg_vl | |
| 11 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI117 C5- | NO: 352 | BI117 | NO: 369 |
| | | hCH1- | | hCLIg_vk | | R2A VH- | | VL- | |
| | | hFc_Knob | | | | hCH1- | | hCLIg_vl | |
| | | | | | | hFc_Hole | | | |
| 12 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI117 C9- | NO: 353 | BI117 | NO: 369 |
| | | hCH1- | | hCLIg_vk | | R2A VH- | | VL- | |
| | | hFc_Knob | | | | hCH1- | | hCLIg_vl | |
| | | | | | | hFc_Hole | | | |
| 13 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI117 D1- | NO: 354 | BI117 | NO: 369 |
| | | hCH1- | | hCLIg_vk | | R2A VH- | | VL- | |
| | | hFc_Knob | | | | hCH1- | | hCLIg_vl | |
| | | | | | | hFc_Hole | | | |
| 14 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI117 E2- | NO: 355 | BI117 | NO: 369 |
| | | hCH1- | | hCLIg_vk | | R2B VH- | | VL- | |
| | | hFc_Knob | | | | hCH1- | | hCLIg_vl | |
| | | | | | | hFc_Hole | | | |
| 15 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI117 G1- | NO: 356 | BI117 | NO: 369 |
| | | hCH1- | | hCLIg_vk | | R2B VH- | | VL- | |
| | | hFc_Knob | | | | hCH1- | | hCLIg_vl | |
| | | | | | | hFc_Hole | | | |
| 16 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI117 G12- | NO: 357 | BI117 | NO: 369 |
| | | hCH1- | | hCLIg_vk | | R2B VH- | | VL- | |
| | | hFc_Knob | | | | hCH1- | | hCLIg_vl | |
| | | | | | | hFc_Hole | | | |
| 17 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI117 D6- | NO: 358 | BI117 | NO: 369 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 18 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI117 A6- | NO: 359 | BI117 | NO: 369 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 19 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI117 A11- | NO: 360 | BI117 | NO: 369 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 20 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI117 B2- | NO: 361 | BI117 | NO: 369 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 21 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI117 C5- | NO: 362 | BI117 | NO: 369 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 22 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI117 C6- | NO: 363 | BI117 | NO: 369 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 23 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI117 D11- | NO: 364 | BI117 | NO: 369 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 24 | BIM0205 | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI123 | NO: 365 | BI123 | NO: 370 |
| | | hCH1- | | hCLIg_vk | | VH-hCH1- | | VL- | |
| | | hFc_Knob | | | | hFc_Hole | | hCLIg_vl | |
| 25 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI123 A5- | NO: 366 | BI123 | NO: 370 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 26 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI123 B6- | NO: 367 | BI123 | NO: 370 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 27 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI123 C1- | NO: 368 | BI123 | NO: 370 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 28 | BIM0206 | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI117 | NO: 351 | BI117 | NO: 369 |
| | | hCH1- | | hCLIg_vk | | VH-hCH1- | | VL- | |
| | | hFc_Knob | | | | hFc_Hole | | hCLIg_vl | |
| 29 | | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI117 C5- | NO: 352 | BI117 | NO: 369 |
| | | hCH1- | | hCLIg_vk | | R2A VH- | | VL- | |
| | | hFc_Knob | | | | hCH1- | | hCLIg_vl | |
| | | | | | | hFc_Hole | | | |
| 30 | | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI117 C9- | NO: 353 | BI117 | NO: 369 |
| | | hCH1- | | hCLIg_vk | | R2A VH- | | VL- | |
| | | hFc_Knob | | | | hCH1- | | hCLIg_vl | |
| | | | | | | hFc_Hole | | | |
| 31 | | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI117 D1- | NO: 354 | BI117 | NO: 369 |
| | | hCH1- | | hCLIg_vk | | R2A VH- | | VL- | |
| | | hFc_Knob | | | | hCH1- | | hCLIg_vl | |
| | | | | | | hFc_Hole | | | |
| 32 | | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI117 E2- | NO: 355 | BI117 | NO: 369 |
| | | hCH1- | | hCLIg_vk | | R2B VH- | | VL- | |
| | | hFc_Knob | | | | hCH1- | | hCLIg_vl | |
| | | | | | | hFc_Hole | | | |
| 33 | | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI117 G1- | NO: 356 | BI117 | NO: 369 |
| | | hCH1- | | hCLIg_vk | | R2B VH- | | VL- | |
| | | hFc_Knob | | | | hCH1- | | hCLIg_vl | |
| | | | | | | hFc_Hole | | | |
| 34 | | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI117 G12- | NO: 357 | BI117 | NO: 369 |
| | | hCH1- | | hCLIg_vk | | R2B VH- | | VL- | |
| | | hFc_Knob | | | | hCH1- | | hCLIg_vl | |
| | | | | | | hFc_Hole | | | |
| 35 | | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI117 D6- | NO: 358 | BI117 | NO: 369 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 36 | | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI117 A6- | NO: 359 | BI117 | NO: 369 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 37 | | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI117 A11- | NO: 360 | BI117 | NO: 369 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 38 | | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI117 B2- | NO: 361 | BI117 | NO: 369 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 39 | | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI117 C5- | NO: 362 | BI117 | NO: 369 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 40 | | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI117 C6- | NO: 363 | BI117 | NO: 369 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 41 | | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI117 D11- | NO: 364 | BI117 | NO: 369 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 42 | BIM0207 | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI123 | NO: 365 | BI123 | NO: 370 |
| | | hCH1- | | hCLIg_vk | | VH-hCH1- | | VL- | |
| | | hFc_Knob | | | | hFc_Hole | | hCLIg_vl | |
| 43 | | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI123 A5- | NO: 366 | BI123 | NO: 370 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 44 | | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI123 B6- | NO: 367 | BI123 | NO: 370 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 45 | | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI123 C1- | NO: 368 | BI123 | NO: 370 |
| | | hCH1- | | hCLIg_vk | | R3B VH- | | VL- | |
| | | hFc_Knob | | | | hCH1- | | hCLIg_vl | |
| | | | | | | hFc_Hole | | | |
| 46 | | aCCR2 | SEQ ID | aCCR2 | SEQ ID | aCSF1R | SEQ ID | aCSF1R | SEQ ID |
| | | Hum1 VH- | NO: 376 | VL- | NO: 373 | BI117 | NO: 351 | BI117 | NO: 369 |
| | | hCH1- | | hCLIg_vk | | VH-hCH1- | | VL- | |
| | | hFc_Knob- | | | | hFc_Hole | | hCLIg_vl | |
| | | 3x4GS- | | | | | | | |
| | | BH794_V | | | | | | | |
| | | H-3x4GS- | | | | | | | |
| | | BH794_VL | | | | | | | |
| 47 | | aCCR2 | SEQ ID | aCCR2 | SEQ ID | aCSF1R | SEQ ID | aCSF1R | SEQ ID |
| | | Hum1 VH- | NO: 376 | VL- | NO: 373 | BI123 | NO: 365 | BI123 | NO: 370 |
| | | hCH1- | | hCLIg_vk | | VH-hCH1- | | VL- | |
| | | hFc_Knob- | | | | hFc_Hole | | hCLIg_vl | |
| | | 3x4GS- | | | | | | | |
| | | BH794_V | | | | | | | |
| | | H-3x4GS- | | | | | | | |
| | | BH794_VL | | | | | | | |
| 48 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI117 | NO: 374 | BI117 | NO: 369 |
| | | hCH1- | | hCLIg_vk | | VH-hCH1- | | VL- | |
| | | hFc_Knob | | | | hFc_Hole- | | hCLIg_vl | |
| | | | | | | 3x4GS- | | | |
| | | | | | | BH794_VH- | | | |
| | | | | | | 3x4GS- | | | |
| | | | | | | BH794_VL | | | |
| 49 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI123 | NO: 375 | BI123 | NO: 370 |
| | | hCH1- | | hCLIg_vk | | VH-hCH1- | | VL- | |
| | | hFc_Knob | | | | hFc_Hole- | | hCLIg_vl | |
| | | | | | | 3x4GS- | | | |
| | | | | | | BH794_VH- | | | |
| | | | | | | 3x4GS- | | | |
| | | | | | | BH794_VL | | | |
| 50 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI117 | NO: 351 | BI117 | NO: 377 |
| | | hCH1- | | hCLIg_vk | | VH-hCH1- | | VL- | |
| | | hFc_Knob | | | | hFc_Hole | | hCLIg_vl- | |
| | | | | | | | | 3x4GS-IL2 | |
| 51 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI123 | NO: 365 | BI123 | NO: 379 |
| | | hCH1- | | hCLIg_vk | | VH-hCH1- | | VL- | |
| | | hFc_Knob | | | | hFc_Hole | | hCLIg_vl- | |
| | | | | | | | | 3x4GS-IL2 | |
| 52 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI117 | NO: 351 | BI117 | NO: 378 |
| | | hCH1- | | hCLIg_vk | | VH-hCH1- | | VL- | |
| | | hFc_Knob | | | | hFc_Hole | | hCLIg_vl- | |
| | | | | | | | | IL2 | |
| 53 | | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI123 | NO: 365 | BI123 | NO: 380 |
| | | hCH1- | | hCLIg_vk | | VH-hCH1- | | VL- | |
| | | hFc_Knob | | | | hFc_Hole | | hCLIg_vl- | |
| | | | | | | | | IL2 | |
| 54 | | aCCR2 | SEQ ID | aCCR2 | SEQ ID | aCSF1R | SEQ ID | aCSF1R | SEQ ID |
| | | Hum1 VH- | NO: 371 | VL- | NO: 381 | BI117 | NO: 351 | BI117 | NO: 369 |
| | | hCH1- | | hCLIg_vk- | | VH-hCH1- | | VL- | |
| | | hFc_Knob | | 3x4GS-IL2 | | hFc_Hole | | hCLIg_vl | |
| 55 | | aCCR2 | SEQ ID | aCCR2 | SEQ ID | aCSF1R | SEQ ID | aCSF1R | SEQ ID |
| | | Hum1 VH- | NO: 371 | VL- | NO: 381 | BI117 | NO: 351 | BI117 | NO: 369 |
| | | hCH1- | | hCLIg_vk- | | VH-hCH1- | | VL- | |
| | | hFc_Knob | | 3x4GS-IL2 | | hFc_Hole | | hCLIg_vl | |
| 56 | | aCCR2 | SEQ ID | aCCR2 | SEQ ID | aCSF1R | SEQ ID | aCSF1R | SEQ ID |
| | | Hum1 VH- | NO: 371 | VL- | NO: 382 | BI123 | NO: 365 | BI123 | NO: 370 |
| | | hCH1- | | hCLIg_vk- | | VH-hCH1- | | VL- | |
| | | hFc_Knob | | IL2 | | hFc_Hole | | hCLIg_vl | |
| 57 | | aCCR2 | SEQ ID | aCCR2 | SEQ ID | aCSF1R | SEQ ID | aCSF1R | SEQ ID |
| | | Hum1 VH- | NO: 371 | VL- | NO: 382 | BI123 | NO: 365 | BI123 | NO: 370 |
| | | hCH1- | | hCLIg_vk- | | VH-hCH1- | | VL- | |
| | | hFc_Knob | | IL2 | | hFc_Hole | | hCLIg_vl | |
| 58 | | aCCR2 | SEQ ID | aCCR2 | SEQ ID | aCSF1R | SEQ ID | aCSF1R | SEQ ID |
| | | Hum1 VH- | NO: 385 | VL- | NO: 373 | BI117 | NO: 383 | BI117 | NO: 369 |
| | | hCH1- | | hCLIg_vk | | VH-hCH1- | | VL- | |
| | | hFc_Knob- | | | | hFc_Hole- | | hCLIg_vl | |
| | | 3x4GS- | | | | 3x4GS- | | | |
| | | TGFbR2 | | | | TGFbR2 | | | |
| 59 | | aCCR2 | SEQ ID | aCCR2 | SEQ ID | aCSF1R | SEQ ID | aCSF1R | SEQ ID |
| | | Hum1 VH- | NO: 385 | VL- | NO: 373 | BI123 | NO: 384 | BI123 | NO: 370 |
| | | hCH1- | | hCLIg_vk | | VH-hCH1- | | VL- | |
| | | hFc_Knob- | | | | hFc_Hole- | | hCLIg_vl | |
| | | 3x4GS- | | | | 3x4GS- | | | |
| | | TGFbR2 | | | | TGFbR2 | | | |
| 60 | | aCCR2 | SEQ ID | aCCR2 | SEQ ID | aCSF1R | SEQ ID | | |
| | | Hum1 VH- | NO: 371 | VL- | NO: 373 | BI117 | NO: 386 | | |
| | | hCH1- | | hCLIg_vk | | VH-3x4GS- | | | |
| | | hFc_Knob | | | | aCSF1R | | | |
| | | | | | | BI117 VL- | | | |
| | | | | | | 4GS- | | | |
| | | | | | | hFc_Hole | | | |
| 61 | | aCCR2 | SEQ ID | aCCR2 | SEQ ID | aCSF1R | SEQ ID | | |
| | | Hum1 VH- | NO: 371 | VL- | NO: 373 | BI123 | NO: 387 | | |
| | | hCH1- | | hCLIg_vk | | VH-3x4GS- | | | |
| | | hFc_Knob | | | | aCSF1R | | | |
| | | | | | | BI123 VL- | | | |
| | | | | | | 4GS- | | | |
| | | | | | | hFc_Hole | | | |
| 62 | | aCCR2 | SEQ ID | aCCR2 | SEQ ID | aCSF1R | SEQ ID | | |
| | | Hum1 VH- | NO: 385 | VL- | NO: 373 | BI117 | NO: 388 | | |
| | | hCH1- | | hCLIg_vk | | VH-3x4GS- | | | |
| | | hFc_Knob- | | | | aCSF1R | | | |
| | | 3x4GS- | | | | BI117 VL- | | | |
| | | TGFbR2 | | | | 4GS- | | | |
| | | | | | | hFc_Hole- | | | |
| | | | | | | 3x4GS- | | | |
| | | | | | | TGFbR2 | | | |
| 63 | | aCCR2 | SEQ ID | aCCR2 | SEQ ID | aCSF1R | SEQ ID | | |
| | | Hum1 VH- | NO: 385 | VL- | NO: 373 | BI123 | NO: 389 | | |
| | | hCH1- | | hCLIg_vk | | VH-3x4GS- | | | |
| | | hFc_Knob- | | | | aCSF1R | | | |
| | | 3x4GS- | | | | BI123 VL- | | | |
| | | TGFbR2 | | | | 4GS- | | | |
| | | | | | | hFc_Hole- | | | |
| | | | | | | 3x4GS- | | | |
| | | | | | | TGFbR2 | | | |
| 64 | | aCCR2 | SEQ ID | aCCR2 | SEQ ID | TGFbR2- | SEQ ID | TGFbR2- | SEQ ID |
| | | Hum1 VH- | NO: 392 | VL- | NO: 373 | 3x4GS- | NO: 390 | 3x4GS- | NO: 391 |
| | | hCH1- | | hCLIg_vk | | hCH1- | | hCLIg_vl | |
| | | hFc_Knob- | | | | hFc_Hole | | | |
| | | 3x4GS- | | | | | | | |
| | | BI117 VH- | | | | | | | |
| | | 3x4GS- | | | | | | | |
| | | aCSF1R | | | | | | | |
| | | BI117 VL | | | | | | | |
| 65 | | aCCR2 | SEQ ID | aCCR2 | SEQ ID | TGFbR2- | SEQ ID | TGFbR2- | SEQ ID |
| | | Hum1 VH- | NO: 393 | VL- | NO: 373 | 3x4GS- | NO: 390 | 3x4GS- | NO: 391 |
| | | hCH1- | | hCLIg_vk | | hCH1- | | hCLIg_vl | |
| | | hFc_Knob- | | | | hFc_Hole | | | |
| | | 3x4GS- | | | | | | | |
| | | BI123 VH- | | | | | | | |
| | | 3x4GS- | | | | | | | |
| | | aCSF1R | | | | | | | |
| | | BI123 VL | | | | | | | |
| 66 | BIM0208 | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI117 | NO: 351 | BI123 | NO: 370 |
| | | hCH1- | | hCLIg_vk | | VH-hCH1- | | VL- | |
| | | hFc_Knob | | | | hFc_Hole | | hCLIg_vl | |
| 67 | BIM0209 | aCCR2 | SEQ ID | aCCR2 | SEQ ID | aCSF1R | SEQ ID | aCSF1R | SEQ ID |
| | | Hum1 VH- | NO: 371 | VL- | NO: 373 | BI123 | NO: 365 | BI117 | NO: 369 |
| | | hCH1- | | hCLIg_vk | | VH-hCH1- | | VL- | |
| | | hFc_Knob | | | | hFc_Hole | | hCLIg_vl | |
| 68 | BIM0210 | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI117 | NO: 351 | BI123 | NO: 370 |
| | | hCH1- | | hCLIg_vk | | VH-hCH1- | | VL- | |
| | | hFc_Knob | | | | hFc_Hole | | hCLIg_vl | |
| 69 | BIM0211 | aCCR2 | SEQ ID | aCCR2 | SEQ ID | aCSF1R | SEQ ID | aCSF1R | SEQ ID |
| | | Hum2 VH- | NO: 372 | VL- | NO: 373 | BI123 | NO: 365 | BI117 | NO: 369 |
| | | hCH1- | | hCLIg_vk | | VH-hCH1- | | VL- | |
| | | hFc_Knob | | | | hFc_Hole | | hCLIg_vl | |
| 70 | BIM0542 | hFc_Knob | SEQ ID | | | aCSF1R | SEQ ID | aCSF1R | SEQ ID |
| | | (H435R, | NO: 401 | | | BI117 C5- | NO: 352 | BI117 | NO: 369 |
| | | Y436F)- | | | | R2A VH- | | VL- | |
| | | 2x4GS- | | | | hCH1- | | hCLIg_vl | |
| | | HHHHHHHH | | | | hFc_Hole | | | |
| 71 | BIM0543 | hFc_Knob | SEQ ID | | | aCSF1R | SEQ ID | aCSF1R | SEQ ID |
| | | (H435R, | NO: 401 | | | BI117 C9- | NO: 353 | BI117 | NO: 369 |
| | | Y436F)- | | | | R2A VH- | | VL- | |
| | | 2x4GS- | | | | hCH1- | | hCLIg_vl | |
| | | HHHHHHHH | | | | hFc_Hole | | | |
| 72 | BIM0544 | hFc_Knob | SEQ ID | | | aCSF1R | SEQ ID | aCSF1R | SEQ ID |
| | | (H435R, | NO: 401 | | | BI117 D1- | NO: 354 | BI117 | NO: 369 |
| | | Y436F)- | | | | R2A VH- | | VL- | |
| | | 2x4GS- | | | | hCH1- | | hCLIg_vl | |
| | | HHHHHHHH | | | | hFc_Hole | | | |
| 73 | BIM0545 | hFc_Knob | SEQ ID | | | aCSF1R | SEQ ID | aCSF1R | SEQ ID |
| | | (H435R, | NO: 401 | | | BI117 E2- | NO: 355 | BI117 | NO: 369 |
| | | Y436F)- | | | | R2B VH- | | VL- | |
| | | 2x4GS- | | | | hCH1- | | hCLIg_vl | |
| | | HHHHHHHH | | | | hFc_Hole | | | |
| 74 | BIM0546 | hFc_Knob | SEQ ID | | | aCSF1R | SEQ ID | aCSF1R | SEQ ID |
| | | (H435R, | NO: 401 | | | BI117 G1- | NO: 356 | BI117 | NO: 369 |
| | | Y436F)- | | | | R2B VH- | | VL- | |
| | | 2x4GS- | | | | hCH1- | | hCLIg_vl | |
| | | HHHHHHHH | | | | hFc_Hole | | | |
| 75 | BIM0547 | hFc_Knob | SEQ ID | | | aCSF1R | SEQ ID | aCSF1R | SEQ ID |
| | | (H435R, | NO: 401 | | | BI117 G12- | NO: 357 | BI117 | NO: 369 |
| | | Y436F)- | | | | R2B VH- | | VL- | |
| | | 2x4GS- | | | | hCH1- | | hCLIg_vl | |
| | | HHHHHHHH | | | | hFc_Hole | | | |
| 76 | BIM0548 | hFc_Knob | SEQ ID | | | aCSF1R | SEQ ID | aCSF1R | SEQ ID |
| | | (H435R, | NO: 401 | | | BI117 D6- | NO: 358 | BI117 | NO: 369 |
| | | Y436F)- | | | | R3B VH- | | VL- | |
| | | 2x4GS- | | | | hCH1- | | hCLIg_vl | |
| | | HHHHHHHH | | | | hFc_Hole | | | |
| 77 | BIM0549 | hFc_Knob | SEQ ID | | | aCSF1R | SEQ ID | aCSF1R | SEQ ID |
| | | (H435R, | NO: 401 | | | BI117 A6- | NO: 359 | BI117 | NO: 369 |
| | | Y436F)- | | | | R3B VH- | | VL- | |
| | | 2x4GS- | | | | hCH1- | | hCLIg_vl | |
| | | HHHHHHHH | | | | hFc_Hole | | | |
| 78 | BIM0550 | hFc_Knob | SEQ ID | | | aCSF1R | SEQ ID | aCSF1R | SEQ ID |
| | | (H435R, | NO: 401 | | | BI117 A11- | NO: 360 | BI117 | NO: 369 |
| | | Y436F)- | | | | R3B VH- | | VL- | |
| | | 2x4GS- | | | | hCH1- | | hCLIg_vl | |
| | | HHHHHHHH | | | | hFc_Hole | | | |
| 79 | BIM0551 | hFc_Knob | SEQ ID | | | aCSF1R | SEQ ID | aCSF1R | SEQ ID |
| | | (H435R, | NO: 401 | | | BI117 B2- | NO: 361 | BI117 | NO: 369 |
| | | Y436F)- | | | | R3B VH- | | VL- | |
| | | 2x4GS- | | | | hCH1- | | hCLIg_vl | |
| | | HHHHHHHH | | | | hFc_Hole | | | |
| 80 | BIM0552 | hFc_Knob | SEQ ID | | | aCSF1R | SEQ ID | aCSF1R | SEQ ID |
| | | (H435R, | NO: 401 | | | BI117 | NO: 351 | BI117 | NO: 369 |
| | | Y436F)- | | | | VH-hCH1- | | VL- | |
| | | 2x4GS- | | | | hFc_Hole | | hCLIg_vl | |
| | | HHHHHHHH | | | | | | | |
| 81 | BIM0553 | hFc_Knob | SEQ ID | | | aCSF1R | SEQ ID | aCSF1R | SEQ ID |
| | | (H435R, | NO: 401 | | | BI117 C5- | NO: 362 | BI117 | NO: 369 |
| | | Y436F)- | | | | R3B VH- | | VL- | |
| | | 2x4GS- | | | | hCH1- | | hCLIg_vl | |
| | | HHHHHHHH | | | | hFc_Hole | | | |
| 82 | BIM0554 | hFc_Knob | SEQ ID | | | aCSF1R | SEQ ID | aCSF1R | SEQ ID |
| | | (H435R, | NO: 401 | | | BI123 A5- | NO: 366 | BI123 | NO: 370 |
| | | Y436F)- | | | | R3B VH- | | VL- | |
| | | 2x4GS- | | | | hCH1- | | hCLIg_vl | |
| | | HHHHHHHH | | | | hFc_Hole | | | |
| 83 | BIM0555 | hFc_Knob | SEQ ID | | | aCSF1R | SEQ ID | aCSF1R | SEQ ID |
| | | (H435R, | NO: 401 | | | BI123 B6- | NO: 367 | BI123 | NO: 370 |
| | | Y436F)- | | | | R3B VH- | | VL- | |
| | | 2x4GS- | | | | hCH1- | | hCLIg_vl | |
| | | HHHHHHHH | | | | hFc_Hole | | | |
| 84 | BIM0556 | hFc_Knob | SEQ ID | | | aCSF1R | SEQ ID | aCSF1R | SEQ ID |
| | | (H435R, | NO: 401 | | | BI123 C1- | NO: 368 | BI123 | NO: 370 |
| | | Y436F)- | | | | R3B VH- | | VL- | |
| | | 2x4GS- | | | | hCH1- | | hCLIg_vl | |
| | | HHHHHHHH | | | | hFc_Hole | | | |
| 85 | BIM0566 | hFc_Knob | SEQ ID | | | aCSF1R | SEQ ID | aCSF1R | SEQ ID |
| | | (H435R, | NO: 401 | | | BI117 C6- | NO: 363 | BI117 | NO: 369 |
| | | Y436F)- | | | | R3B VH- | | VL- | |
| | | 2x4GS- | | | | hCH1- | | hCLIg_vl | |
| | | HHHHHHHH | | | | hFc_Hole | | | |
| 86 | BIM0567 | hFc_Knob | SEQ ID | | | aCSF1R | SEQ ID | aCSF1R | SEQ ID |
| | | (H435R, | NO: 401 | | | BI117 D11- | NO: 364 | BI117 | NO: 369 |
| | | Y436F)- | | | | R3B VH- | | VL- | |
| | | 2x4GS- | | | | hCH1- | | hCLIg_vl | |
| | | HHHHHHHH | | | | hFc_Hole | | | |

TABLE 34
Exemplary UniTI-102 molecules.
BIM0648

| | | |
|---|---|---|
| SEQ ID | aCSF1R BI117 | GFTFSNDEMN |
| NO: 402 | C5-R2A | |
| | HCDR1 | |
| SEQ ID | aCSF1R BI117 | YISSGSSTIYYADAVKG |
| NO: 405 | C5-R2A | |
| | HCDR2 | |
| SEQ ID | aCSF1R BI117 | GVDGDFSFDY |
| NO: 413 | C5-R2A | |
| | HCDR3 | |
| SEQ ID | aCSF1R BI117 | EVQLVESGGGLVQPGGSLRLSCAASGFTFSDEMNWIRQAPGKGLEWV |
| NO: 324 | C5-R2A VH aa | SYISSGSSTITTADAVKGRFTISRDNARNSLYLQMNSLRATETAVYYCAR |
| | | GVDGDFSFDYWGQGTLVTVSS |
| SEQ ID | aCSF1R BI117 | GAAGTGCAGCTGGTTGAATCTGGCGGAGGACTGGTTCAGCCTGGCGG |
| NO: 129 | C5-R2A VH nt | ATCTCTGAGACTGTCTTGTGCCGCCTCTGGCTTCACCTTCTCCAACGA |
| | | CGAGATGAACTGGATCAGACAGGCCCCTGGCAAAGGCCTGGAATGG |
| | | GTGTCCTACATCTCCTCCGGCTCCTCCACCATCTACTACGCCGATGCT |
| | | GTGAAGGGCAGATTCACCATCTCTCGGGACAACGCCCGGAACTCCCT |
| | | GTACCTGCAGATGAACTCTCTGAGAGCCGAGGACACCGCCGTGTACT |
| | | ACTGTGCTAGAGGCGTGGACGGCGACTTCTCCTTCGATTATTGGGGOC |
| | | AGGGCACCCTGGTCACAGTTTCTAGC |
| SEQ ID | aCSF1R BI117 | TGSSSNIGAGYDVH |
| NO: 432 | LCDR1 | |
| SEQ ID | aCSF1R BI117 | GNSNRPS |
| NO: 434 | LCDR2 | |
| SEQ ID | aCSF1R BI117 | QSYDSSLSEEV |
| NO: 436 | LCDR3 | |
| SEQ ID | aCSF1R BI117 | QLVLTQPPSVSGAPGQRVTISCTGSSSNIGAGYDVHWYQQLPGTAPKLLI |
| NO: 341 | VL aa | YGNSNRPSGVPDRFSGSKSGTSASLAITGLQAEDEADYYCQSYDSSLSEE |
| | | VFGGGTKLTVL |
| SEQ ID | aCSF1R BI117 | CAGCTGGTTTTGACCCAGCCTCCTTCCGTGTCTGGTGCTCCTGGACAG |
| NO: 130 | VL nt | AGAGTGACCATCTCTTGCACCGGCTCCAGCTCCAATATCGGCGCTGGC |
| | | TACGACGTGCACTGGTATCAGCAGCTGCCTGGCACAGCTCCCAAACT |
| | | GCTGATCTACGGCAACTCCAACCGGCCTTCTGGCGTGCCCGATAGATT |
| | | CTCCGGCTCTAAGTCTGGCACCTCTGCCAGCCTGGCTATCACTGGACT |
| | | GCAGGCTGAGGACGAGGCCGACTACTACTGCCAGTCCTACGACTCCA |
| | | GCCTGTCCGAGGAAGTTTTTGGCGGAGGCACCAAGCTGACCGTGCTG |
| SEQ ID | aCSF1R BI117 | EVQLVESGGGLVQPGGSLRLSCAASGFTFSNDEMNWIRQAPGKGLEWV |
| NO: 127 | C5-R2A VH- | SYISSGSSTIYYADAVKGRFTISRDNARNSLYLQMNSLRAEDTAVYYCAR |
| | 4x4GS-aCSF1R | GVDGDFSFDYWGQGTLVTVSSGGGGSGGGGSGGGGSGGGGSQLVLTQP |
| | BI117 VL- | PSVSGAPGQRVTISCTGSSSNIGAGYDVHWYQQLPGTAPKLLIYGNSNRP |
| | hFc_Knob- | SGVPDRFSGSKSGTSASLAITGLQAEDEADYYCQSYDSSLSEEVFGGGTK |
| | 4x4GS- | LTVLDKTHTCPPCPAPELLGGPSVFLFPPKPKDTLIMSRTPEVTCVVVDVS |
| | TGFbR2 aa | HEDPEVKFNWYVDGVEVHNAKTKPREEQYNSTYRVVSVLTVLHQDWL |
| | | NGKEYKCKVSNKALPAPIEKTISKAKGQPREPQVYTLPPCREEMTKNQV |
| | | SLWCLVKGFYPSDIAVEWESNGQPENNYKTTPPVLDSDGSFFLYSKLTV |
| | | DKSRWQQGNVFSCSVMHEALHNHYTQKSLSLSPGKGGGGSGGGGSGG |
| | | GGSGGGGSIPPHVQKSVNNDMIVTDNNGAVKFPQLCKFCDVRFSTCDNQ |
| | | KSCMSNCSITSICEKPQEVCVAVWRKNDENITLETVCHDPKLPYHDFILE |
| | | DAASPKCIMKEKKKPGETFFMCSCSSDECNDNIIFSEEYNTSNPD |
| SEQ ID | aCSF1R BI117 | GAAGTGCAGCTGGTTGAATCTGGCGGAGGACTGGTTCAGCCTGGCGG |
| NO: 128 | C5-R2A VH- | ATCTCTGAGACTGTCTTGTGCCGCCTCTGGCTTCACCTTCTCCAACGA |
| | 4x4GS-aCSF1R | CGAGATGAACTGGATCAGACAGGCCCCTGGCAAAGGCCTGGAATGG |
| | BI117 VL- | GTGTCCTACATCTCCTCCGGCTCCTCCACCATCTACTACGCCGATGCT |
| | hFc_Knob- | GTGAAGGGCAGATTCACCATCTCTCGGGACAACGCCCGGAACTCCCT |
| | 4x4GS- | GTACCTGCAGATGAACTCTCTGAGAGCCGAGGACACCGCCGTGTACT |
| | TGFbR2 nt | ACTGTGCTAGAGGCGTGGACGGCGACTTCTCCTTCGATTATTGGGGCC |
| | | AGGGCACCCTGGTCACAGTTTCTAGCGGAGGCGGAGGTTCTGGCGGC |
| | | GGTGGAAGTGGCGGTGGTGGATCAGGCGGCGGAGGATCTCAGCTGGT |
| | | TTTGACCCAGCCTCCTTCCGTGTCTGGTGCTCCTGGACAGAGAGTGAC |
| | | CATCTCTTGCACCGGCTCCAGCTCCAATATCGGCGCTGGCTACGACGT |
| | | GCACTGGTATCAGCAGCTGCCTGGCACAGCTCCCAAACTGCTGATCT |
| | | ACGGCAACTCCAACCGGCCTTCTGGCGTGCCCGATAGATTCTCCGGCT |
| | | CTAAGTCTGGCACCTCTGCCAGCCTGGCTATCACTGGACTGCAGGCTG |
| | | AGGACGAGGCCGACTACTACTGCCAGTCCTACGACTCCAGCCTGTCC |
| | | GAGGAAGTTTTTGGCGGAGGCACCAAGCTGACCGTGCTGGACAAGAC |
| | | CCATACCTGTCCTCCATGTCCTGCTCCAGAGCTGCTCGGAGGCCCTTC |
| | | TGTGTTCCTGTTTCCTCCAAAGCCTAAGGACACCCTGATGATCTCTCG |
| | | GACCCCTGAAGTGACCTGCGTGGTGGTCGATGTGTCTCACGAGGATC |
| | | CCGAAGTGAAGTTCAATTGGTACGTCGACGGCGTCGAGGTGCACAAC |
| | | GCTAAGACCAAGCCTAGAGAGGAACAGTACAACTCCACCTACAGAGT |
| | | GGTGTCCGTGCTGACAGTGCTGCACCAGGATTGGCTGAACGGCAAAG |
| | | AGTACAAGTGCAAGGTGTCCAACAAGGCCCTGCCTGCTCCTATCGAA |
| | | AAGACCATCTCCAAGGCCAAGGGCCAGCCTAGGGAACCCCAGGTTTA |
| | | CACCCTGCCTCCATGCCGGGAAGAGATGACCAAGAACCAGGTGTCCC |
| | | TGTGGTGCCTGGTCAAGGGCTTCTACCCTTCCGATATCGCCGTGGAAT |
| | | GGGAGAGCAATGGCCAGCCTGAGAACAACTACAAGACCACACCTCCT |
| | | GTGCTGGACTCCGACGGCTCATTCTTCCTGTACAGCAAGCTGACTGTG |
| | | GATAAGTCCCGGTGGCAGCAGGGCAACGTGTTCTCCTGTTCTGTGATG |
| | | CACGAGGCCCTGCACAACCACTACACCCAGAAGTCCCTGTCTCTGTCT |
| | | CCCGGAAAAGGTGGTGGTGGTAGTGGTGGCGGTGGCTCTGGTGGTGG |
| | | CGGATCCGGCGGAGGCGGTTCTATTCCTCCACACGTGCAGAAATCCG |
| | | TGAACAACGACATGATCGTGACCGACAACAATGGCGCCGTGAAGTTC |
| | | CCTCAGCTGTGCAAGTTTTGCGACGTGCGGTTCTCTACCTGCGACAAC |
| | | CAGAAATCCTGCATGTCCAACTGCTCCATCACCTCCATCTGCGAGAAG |
| | | CCCCAAGAAGTGTGCGTCGCCGTCTGGCGGAAGAACGATGAGAACAT |
| | | CACCCTGGAAACCGTGTGCCACGATCCTAAGCTGCCCTACCACGACTT |
| | | CATCCTGGAAGATGCCGCTTCTCCCAAGTGCATCATGAAGGAAAAGA |
| | | AGAAGCCCGGCGAGACTTTCTTCATGTGCAGCTGCTCCTCCGACGAGT |
| | | GCAACGACAACATCATCTTCTCCGAAGAGTATAACACCAGCAATCCC |
| | | GAC |
| SEQ ID | aCCR2 Hum1 | GFSFNAYAMN |
| NO: 446 | HCDR1 | |
| SEQ ID | aCCR2 Hum1 | RIRTKNNNYATYYADSVKD |
| NO: 447 | HCDR2 | |
| SEQ ID | aCCR2 Hum1 | FYGNGV |
| NO: 448 | HCDR3 | |
| SEQ ID | aCCR2 Hum1 | EVQLVESGGGLVKPGGSLRLSCAASGFSFNAYAMNWVRQAPGKGLEW |
| NO: 343 | VH aa | VGRIRTKNNNYATYYADSVKDRFTISRDDSKSTLYLQMNSLK1EDTAVY |
| | | YCTTFYGNGVWGQGTTVTVSS |
| SEQ ID | aCCR2 Hum1 | GAAGTGCAGCTGGTTGAATCTGGCGGCGGACTGGTTAAGCCTGGCGG |
| NO: 133 | VH nt | ATCTCTGAGACTGTCTTGTGCCGCCTCCGGCTTCTCCTTCAATGCCTAC |
| | | GCCATGAACTGGGTOCGACAGGCTCCTGGCAAAGGCCTGGAATGGGT |
| | | CGGAAGAATCCGGACCAAGAACAACAACTACGCCACCTACTACGCCG |
| | | ACTCCGTGAAGGACCGGTTCACCATCTCTCGGGACGACTCCAAGAGC |
| | | ACCCTGTACCTGCAGATGAACTCCCTGAAAACCGAGGACACCGCCGT |
| | | GTACTACTGCACCACCTTCTACGGCAATGGCGTGTGGGGCCAGGGCA |
| | | CAACAGTGACAGTCTCTTCC |
| SEQ ID | aCCR2 Hum1 | EVQLVESGGGLVKPGGSLRLSCAASGFSFNAYAMNWVRQPGKGLEW |
| NO: 131 | VH-hCH1- | VGRIRTKNNNYATYYADSVKDRFTISRDDSKSTLYLQMNSLKTEDTAVY |
| | hFc_Hole- | YCTTFYGNGVWGQGTTVTVSASTKGPSVFPLAPSSKSTSGGTAALGCL |
| | 4x4GS- | VKDYFPEPVTVSWNSGALTSGVHTFPAVLQSSGLYSLSSVVTVPSSSLGT |
| | TGFbR2 aa | QTYICNVNHKPSNTKVDKRVEPKSCDKTHTCPPCPAPELLGGPSVFLFPP |
| | | KPKDTLMISRTPEVTCVVVDVSHEDPEVKFNWYVDGVEVHNAKTKPRE |
| | | EQYNSTYRVVSVLTVLHQDWLNGKEYKCKVSNKALPAPIEKTISKAKGQ |
| | | PREPQVCTLPPSREEMTKNQVSLSCAVKGFYPSDIAVEWESNGQPENNY |
| | | KTTPPVLDSDGSFFLVSKLTVDKSRWQQGNVFSCSVMHEALHNHYTQK |
| | | SLSLSPGKGGGGSGGGGSGGGGSGGGGSIPPHVQKSVNNDMIVTDNNGA |
| | | VKFPQLCKFCDVRFSTCDNQKSCMSNCSITSICEKPQEVCVAVWRKNDE |
| | | NITLETVCHDPKLPYHDFILEDAASPKCIMKEKKKPGETFFMCSCSSDEC |
| | | NDNIIFSEEYNTSNPD |
| SEQ ID | aCCR2 Hum1 | GAAGTGCAGCTGGTTGAATCTGGCGGCGGACTGGTTAAGCCTGGCGG |
| NO: 132 | VH-hCH1- | ATCTCTGAGACTGTCTTGTGCCGCCTCCGGCTTCTCCTTCAATGCCTAC |
| | hFc_Hole- | GCCATGAACTGGGTCCGACAGGCTCCTGGCAAAGGCCTGGAATGGGT |
| | 4x4GS- | CGGAAGAATCCGGACCAAGAACAACAACTACGCCACCTACTACGCCG |
| | TGFbR2 nt | ACTCCGTGAAGGACCGGTTCACCATCTCTCGGGACGACTCCAAGAGC |
| | | ACCCTGTACCTGCAGATGAACTCCCTGAAAACCGAGGACACCGCCGT |
| | | GTACTACTGCACCACCTTCTACGGCAATGGCGTGTGGGGCCAGGGCA |
| | | CAACAGTGACAGTCTCTTTCCGCCTTCCACCAAGGGACCCTCTGTGTTTC |
| | | CTCTGGCTCCCTCCAGCAAGTCTACCTCTGGTGGAACAGCTGCCCTGG |
| | | GCTGCCTGGTCAAGGATTACTTTTCCTGAGCCTGTGACCGTGTCCTGGA |
| | | ACTCTGGCGCTCTGACATCTGGCGTGCACACCTTTTCCAGCTGTGCTGC |
| | | AGTCCTCCGGCCTGTACTCTCTGTCCTCTGTCGTGACCGTGCCTTCCA |
| | | GCTCTCTGGGCACCCAGACCTACATCTGCAATGTGAACCACAAGCCTT |
| | | CCAACACCAAGGTGGACAAGAGAGTGGAACCCAAGTCCTGCGACAA |
| | | GACCCACACCTGTCCTCCATGTCCTGCTCCAGAACTGCTCGGCGGACC |
| | | TTCCGTGTTCCTGTTTCCTCCAAAGCCTAAGGACACCCTGATGATCTC |
| | | TCGGACCCCTGAAGTGACCTGCGTGGTGGTGGATGTGTCTCACGAGG |
| | | ATCCCGAAGTGAAGTTCAATTGGTACGTGGACGGCGTGGAAGTGCAC |
| | | AACGCCAAGACCAAGCCTAGAGAGGAACAGTACAACTCCACCTACA |
| | | GAGTGGTGTCCGTGCTGACCGTGCTGCACCAGGATTGGCTGAACGGC |
| | | AAAGAGTACAAGTGCAAGGTGTCCAACAAGGCCCTGCCTGCTCCTAT |
| | | CGAAAAGACCATCTCCAAGGCCAAGGGCCAGCCTCGGGAACCTCAAG |
| | | TCTGTACCCTGCCTCCTAGCCGGGAAGAGATGACCAAGAATCAGGTG |
| | | TCCCTGTCCTGCGCCGTGAAGGGCTTCTACCCTTCTGATATCGCCGTG |
| | | GAATGGGAGAGCAACGGCCAGCCTGAGAACAACTACAAGACAACCC |
| | | CTCCTGTGCTGGACTCCGACGGCTCATTCTTCCTGGTGTCCAAGCTGA |
| | | CAGTGGACAAGTCCAGATGGCAGCAGGGCAACGTGTTCTCCTGCTCC |
| | | GTGATGCACGAGGCCCTGCACAATCACTACACCCAGAAGTCCCTGTC |
| | | TCTGAGCCCAGGCAAAGGTGGCGGAGGATCTGGCGGAGGTGGTAGC |
| | | GGCGGAGGCGGATCAGGCGGTGGTGGCTCTATTCCTCCACACGTGCA |
| | | GAAATCCGTGAACAACGACATGATCGTGACCGACAACAACGGCGCTG |
| | | TGAAGTTCCCTCAGCTGTGCAAGTTCTGCGACGTGCGGTTCTCCACCT |
| | | GTGACAACCAGAAATCCTGCATGTCCAACTGCTCCATCACCTCCATCT |
| | | GCGAGAAGCCCCAAGAAGTGTGCGTCGCCGTCTGGCGGAAGAACGA |
| | | CGAGAACATCACCCTGGAAACCGTGTGCCACGATCCTAAGCTGCCCT |
| | | ACCACGACTTCATCCTGGAAGATGCCGCCTCTCCTAAGTGCATCATGA |
| | | AGGAAAAGAAGAAGCCCGGCGAGACTTTCTTCATGTGCAGCTGCTCC |
| | | TCCGACGAGTGCAACGACAACATCATCTTCTCCGAAGAGTATAACAC |
| | | CAGCAATCCCGAC |
| SEQ ID | aCCR2 VL | KSSQSLLDSDGKTFLN |
| NO: 454 | LCDR1 | |
| SEQ ID | aCCR2 VL | LVSKLDS |
| NO: 455 | LCDR2 | |
| SEQ ID | aCCR2 VL | WQGTHFPYT |
| NO: 456 | LCDR3 | |
| SEQ ID | aCCR2 VL aa | DVVMTQSPLSLPVTLGQPASISCKSSQSLLDSDGKTFLNWLQQRPGQSPR |
| NO: 345 | | RLIYLVSKLDSGVPDRFSGSGSGTDFTLKISRVEAEDVGVYYCWQGTHFP |
| | | YTFGGGTKVEIK |
| SEQ ID | aCCR2 VL nt | GACGTCGTCATGACCCAATCCCCGCTCTCCCTGCCGGTGACTCTCGGC |
| NO: 272 | | CAGCCGGCGTCCATCTCGTGCAAGTCCTCCCAATCCCTGCTGGATTCG |
| | | GATGGGAAGACTTTCCTGAACTGGCTGCAGCAACGGCCCGGCCAGTC |
| | | GCCACGGCGGCTGATCTACCTGGTGTCCAAGCTGGATTCAGGTGTGC |
| | | CTGATAGATTCTCCGGCAGCGGATCGGGGACCGACTTCACGCTGAAG |
| | | ATCTCCAGAGTGGAAGCAGAGGACGTGGGCGTCTATTACTGTTGGCA |
| | | GGGGACTCATTTCCCCTATACCTTTGGAGGTGGTACTAAGGTGGAAAT |
| | | TAAG |
| SEQ ID | aCCR2 VL- | DVVMTQSPLSLPVTLGQPASISCKSSQSLLDSDGKTFLNWLQQRPGQSPR |
| NO: 373 | hCLIg_vk aa | RLIYLVSKLDSGVPDRFSGSGSGTDFTLKISRVEAEDVGVYYCWQGTHFP |
| | | YTFGGGTKVEIKRTVAAPSVFIFPPSDEQLKSGTASVVCLLNNFYPREAK |
| | | VQWKVDNALQSGNSQESVTEQDSKDSTYSLSSTLTLSKADYEKHKVYA |
| | | CEVTHQGLSSPVTKSFNRGEC |
| SEQ ID | aCCR2 VL- | GACGTCGTCATGACCCAATCCCCGCTCTCCCTGCCGGTGACTCTCGGC |
| NO: 134 | hCLIg_vk nt | CAGCCGGCGTCCATCTCGTGCAAGTCCTCCCAATCCCTGCTGGATTCG |
| | | GATGGGAAGACTTTCCTGAACTGGCTGCAGCAACGGCCCGGCCAGTC |
| | | GCCACGGCGGCTGATCTACCTGGTGTCCAAGCTGGATTCAGGTGTGC |
| | | CTGATAGATTCTCCGGCAGCGGATCGGGGACCGACTTCACGCTGAAG |
| | | ATCTCCAGAGTGGAAGCAGAGGACGTGGGCGTCTATTACTGTTGGCA |
| | | GGGGACTCATTTCCCCTATACCTTTGGAGGTGGTACTAAGGTGGAAAT |
| | | TAAGAGAACAGTGGCCGCTCCTTCCGTGTTCATCTTCCCACCCTCCGA |
| | | CGAGCAGCTGAAGTCCGGCACCGCTTCTGTCGTGTGCCTGCTCAACA |
| | | ACTTCTACCCTCGGGAAGCCAAGGTGCAGTGGAAGGTGGACAACGCC |
| | | CTGCAGTCCGGCAACTCCCAGGAATCCGTCACCGAGCAGGACTCCAA |
| | | GGACAGCACCTACTCCCTGTCCTCCACCCTGACCCTGTCCAAGGCCGA |
| | | CTACGAGAAGCACAAGGTGTACGCCTGCGAAGTGACCCACCAGGGCC |
| | | TGAGCAGCCCCGTGACCAAGTCCTTCAACCGGGGCGAGTGC |

BIM0652

| | | |
|---|---|---|
| SEQ ID | aCSF1R BI123 | GFTFSSYAMH |
| NO: 403 | B6-R3B | |
| | HCDR1 | |
| SEQ ID | aCSF1R BI123 | VIPYYGSNKYYADSVKG |
| NO: 411 | B6-R3B | |
| | HCDR2 | |
| SEQ ID | aCSF1R BI123 | ELDYGDYGLDV |
| NO: 422 | B6-R3B | |
| | HCDR3 | |
| SEQ ID | aCSF1R BI123 | QVQLVQSGGGVVQPGRSLRLSCAASGFTFSSYAMHWVRQAPGKGLEW |
| NO: 339 | B6-R3B VH aa | VAVIPYYGSNKYYADSVKGRFTISRDNSKNTLYLQMNSLRAEDTAVYYC |
| | | ARELDYGDYGLDVWGKGTTVTVSS |
| SEQ ID | aCSF1R BI123 | CAGGTGCAGCTGGTTCAATCTGGCGGCGGAGTTGTGCAGCCTGGCAG |
| NO: 138 | B6-R3B VH nt | ATCTCTGAGACTGTCTTGTGCCGCCTCCGGCTTCACCTTCTCCTCTTAC |
| | | GCTATGCACTGGGTCCGACAGGCCCCTGGCAAAGGATTGGAATGGGT |
| | | CGCCGTGATTCCCTACTACGGCTCCAACAAGTACTACGCCGACTCCGT |
| | | GAAGGGCAGATTCACCATCTCTCGGGACAACTCCAAGAACACCCTGT |
| | | ACCTGCAGATGAACTCCCTGAGAGCCGAGGACACCGCCGTGTACTAC |
| | | TGTGCCAGAGAGCTGGACTACGGCGATTACGGCCTGGATGTGTGGGG |
| | | CAAGGGCACCACAGTGACAGTTTCTAGC |
| SEQ ID | aCSF1R BI123 | GGNNIGSKSVH |
| NO: 433 | LCDR1 | |
| SEQ ID | aCSF1R BI123 | DDSDRPS |
| NO: 435 | LCDR2 | |
| SEQ ID | aCSF1R BI123 | QVWDSSSDHVV |
| NO: 437 | LCDR3 | |
| SEQ ID | aCSF1R BI123 | SYVLTQPPSVSVAPGKTARITCGGNNIGSKSVHWYQQKPGQAPVLVVYD |
| NO: 139 | VL aa | DSDRPSGIPERFSGSNGNTATLTISRVEAGDEADYYCQVWDSSSDHVVF |
| | | GGGTQLTVL |
| SEQ ID | aCSF1R BI123 | TCTTACGTGCTGACCCAGCCTCETTCCGTGTCTGTGGCTCCTGGCAAG |
| NO: 140 | VL nt | ACCGCCAGAATCACCTGTGGCGGCAACAACATCGGCTCCAAGTCCGT |
| | | GCACTGGTATCAGCAGAAGCCAGGCCAGGCTCCTGTGCTGGTGGTGT |
| | | ACGACGACTCCGATAGACCCTCTGGCATCCCTGAGCGGTTCTCCGGCT |
| | | CTAACTCTGGCAACACCGCTACACTGACCATCTCCAGAGTGGAAGCT |
| | | GGCGACGAGGCCGACTACTACTGCCAAGTGTGGGACTCCTCCTCCGA |
| | | TCACGTGGTGTTTGGCGGAGGCACACAGCTGACCGTGCTG |
| SEQ ID | aCSF1R BI123 | QVQLVQSGGGVVQPSRSLRLSCAASGFTFSSYAMHWVRQAPGKGLEW |
| NO: 136 | B6-R3B VH- | VAVIPYYGSNKYYADSVKGRFTISRDNSKNTLYLQMNSLRAEDTAVYYC |
| | 4x4GS-aCSF1R | ARELDYGDYGLDVWGKGTTVTVSSGGGGSGGGGSGGGGSGGGGSSYV |
| | BI123 VL- | LTQPPSVSVAPGKTARITCGGNNIGSKSVHWYQQKPGQAPVLVVYDDSD |
| | hFc_Knob- | RPSGIPERFSGSNSGNTATLTISRVEAGDEADYYCQVWDSSSDHVVFGGG |
| | 4x4GS- | TQLTVLDKTHTCPPCPAPELLGGPSVFLFPPKPKDTLMISRTPEVTCVVVD |
| | TGFbR2 aa | VSHEDPEVKFNWYVDGVEVHNAKTKPREEQYNSTRYVVSVLTVLHQD |
| | | WLNGKEYKCKVSNKALPAPIEKTISKAKGQPREPQVYTLPPCREEMTKN |
| | | QVSLWCLVKGFYPSDIAVEWESNGQPENNYKTTPPVLDSDGSFFLYSKL |
| | | TVDKSRWQQGNVFSCSVMHEALHNHYTQKSLSLSPGKGGGGSGGGGSG |
| | | GGGSGGGGSIPPHVQKSVNNDMIVTDNNGAVKFPQLCKFCDVRFSTCDN |
| | | QKSCMSNCSITSICEKPQEVCVAVWRKNDENITLETVCHDPKLPYHDFIL |
| | | EDAASPKCIMKEKKKPGETFFMCSCSSDECNDNIFFSEEYNTSNPD |
| SEQ ID | aCSF1R BI123 | CAGGTGCAGCTGGTTCAATCTGGCGGCGGACTTGTGCAGCCTGGCAG |
| NO: 137 | B6-R3B VH- | ATCTCTGAGACTGTCTTGTGCCGCCTCCGGCTTCACCTTCTCCTCTTAC |
| | 4x4GS-aCSF1R | CCTATGCACTGGGTCCGACAGGCCCCTGGCAAAGGATTGGAATGGGT |
| | BI123 VL- | CGCCGTGATTCCCTACTACGGCTCCAACAAGTACTACGCCGACTCCGT |
| | hFc_Knob- | GAAGGGCAGATTCACCATCTCTCGGGACAACTCCAAGAACACCC1GT |
| | 4x4GS- | ACCTGCAGATGAACTCCCTGAGAGCCGAGGACACCGCCGTGrACTAC |
| | TGFbR2 nt | TGTGCCAGAGAGCTGGACTACGGCGATTACGGCCTGGATGTGTGGGG |
| | | CAAGGGCACCACAGTGACAGTTTCTAGCGGAGGCGGAGGATCAGGTG |
| | | GTGGTGGATCAGGCGGCGGTGGTAGTGGTGGCGGTGGTTCTTCTTAC |
| | | GTGCTGACCCAGCCTCCTTCCGTGTCTGTGGCTCCTGGCAAGACCGCC |
| | | AGAATCACCTGTGGCGGCAACAACATCGGCTCCAAGTCCGTGCACTG |
| | | GTATCAGGAGAAGCCAGGCCAGGCTCCTGTGCTGGTGGTGTACGACG |
| | | ACTCCGATAGACCCTCTGGCATCCCTGAGCGGTTCTCCGGCTCTAACT |
| | | CTGGCAACACCGCTACACTGACCATCTCCAGAGTGGAAGCTGGCGAC |
| | | GAGGCCGACTACTACTGCCAAGTGTGGGACTCCTCCTCCGATCACGT |
| | | GGTGTTTGGCGGAGGCACACAGCTGACCGTGCTGGACAAGACCCATA |
| | | CCTGTCCTCCATGTCCTGCTCCAGAGCTGCTCGGAGGCCCTTCTGTGT |
| | | TCCTGTTTCCTCCAAAGCCTAAGGACACCCTGATGATCTCTCGGACCC |
| | | CTGAAGTGACCTGCGTGGTGGTCGATGTGTCTCACGAGGATCCCGAA |
| | | GTGAAGTTCAATTGGTACGTGGACGGCGTGGAAGTGCACAACGCCAA |
| | | GACCAAGCCTAGAGAGGAACAGTACAACTCCACCTACAGAGTGGTGT |
| | | CCGTGCTGACAGTGCTGCACCAGGATTGGCTGAACGGCAAAGAGTAC |
| | | AAGTGCAAGGTGTCCAACAAGGCCCTGCCTGCTCCTATCGAAAAGAC |
| | | CATCAGCAAGGCCAAGGGCCAGCCTAGGGAACCCCAGGTTTACACCC |
| | | TGCCTCCATGCCGGGAAGAGATGACCAAGAACCAGGTGTCCCTGTGG |
| | | TGTCTGGTCAAGGGCTTCTACCCCTCCGATATCGCCGTGGAATGGGAG |
| | | TCTAATGGCCAGCCTGAGAACAACTACAAGACCACACCTCCAGTGCT |
| | | GGACTCCGACGGCTCATTCTTCCTGTACTCCAAGCTGACTGTGGACAA |
| | | GTCCAGATGGCAGCAGGGCAACGTGTTCTCCTGCTCCGTGATGCACG |
| | | AGGCCCTGCACAATCACTACACCCAGAAGTCCCTGTCTCTGTCTCCCG |
| | | GAAAAGGTGGCGGAGGATCTGGCGGAGGCGGAAGTGGCGGTGGrGG |
| | | AAGCGGTGGTGGCGGATCTATTCCTCCACACGTGCAGAAATCCGTGA |
| | | ACAACGACATGATCGTGACCGACAACAATGGCGCCGTGAAGTTCCCT |
| | | CAGCTGTGCAAGTTCTGCGACGTGCGGTTCTCTACCTGCGACAACCAG |
| | | AAATCCTGCATGTCCAACTGCTCCATCACCTCCATCTGCGAGAAGCCC |
| | | CAAGAAGTGTGCGTCGCCGTCTGGCGGAAGAACGACGAGAACATCAC |
| | | CCTGGAAACAGTGTGCCACGATCCTAAGCTGCCCTACCACGACTTCAT |
| | | CCTGGAAGATGCCGCCTCTCCGTGCATCATGAAGGAAAAGAAGA |
| | | AGCCCGGCGAGACTTTCTTCATGTGCAGCTGCTCCTCCGACGAGTGCA |
| | | ACGACAACATCATCTTCTCCGAAGAGTATAACACCAGCAATCCCGAC |
| SEQ ID | aCCR2 Hum1 | GFSFNAYAMN |
| NO: 446 | HCDR1 | |
| SEQ ID | aCCR2 Hum1 | RIRTKNNNYATYYADSVKD |
| NO: 447 | HCDR2 | |
| SEQ ID | aCCR2 Hum1 | FYGNGV |
| NO: 448 | HCDR3 | |
| SEQ ID | aCCR2 Hum1 | EVQLVESGGGLVKPGGSLRLSCAASGFSFNAYAMNWVRQAPGKGLEW |
| NO: 343 | VH aa | VGRIRTKNNNYATYYADSVKDRFTISRDDSKSTLYLQMNSLKTEDTAVY |
| | | YCTTFYGNGVWGQGTTVTVSS |
| SEQ ID | aCCR2 Hum1 | GAAGTGCAGCTGGTTGAATCTGGCGGCGGACTGGTTAAGCCTGGCGG |
| NO: 133 | VH nt | ATCTCTGAGACTGTCTTGTGCCGCCTCCGGCTTCTCCTTCAATGCCIAC |
| | | GCCATGAACTGGGTCCGACAGGCTCCTGGCAAAGGCCTGGAATGGGT |
| | | CGGAAGAATCCGGACCAAGAACAACAACTACGCCACCTACTACGCCG |
| | | ACTCCGTGAAGGACCGGTTCACCATCTCTCGGGACGACTCCAAGAGC |
| | | ACCCTGTACCTGCAGATGAACTCCCTGAAAACCGAGGACACCGCCGT |
| | | GTACTACTGCACCACCTTCTACGGCAATGGCGTGTGGGGCCAGGGCA |
| | | CAACAGTGACAGTCTCTTCC |
| SEQ ID | aCCR2 Hum1 | EVQLVESGGGLVKPGGSLRLSCAASGFSFNAYAMNWVRQAPGKGLEW |
| NO: 131 | VH-hCH1- | VGRIRTKNNNYATYYADSVKDRFTISRDDSKSTLYLQMNSLKTEDTAVY |
| | hFc_Hole- | YCTTFYGNGVWGQGTTVTVSSASTKGPSVFPLAPSSKSTSGGTAALGCL |
| | 4x4GS- | VKDYFPEPVTVSWNSGALTSGVHTFPAVLQSSGLYSLSSVVTVPSSSLGT |
| | TGFbR2 aa | QTYICNVNHKPSNTKVDKRVEPKSCDKTHTCPPCPAPELLGGPSVFLFPP |
| | | KPKDTLMISRTPEVTCVVVDVSHEDPEVKFNWYVDGVEVHNAKTKPRE |
| | | EQYNSTYRVVSVLTVLHQDWLNGKEYKCKVSNKALPAPIEKTISKAKGQ |
| | | PREPQVCTLPPSREEMTKNQVSLSCAVKGFYPSDIAVEWESNGQPENNY |
| | | KTTPPVLDSDGSFFLVSKLTVDKSRWQQGNVFSCVMHEALHNHYTQK |
| | | SLSLSPGKGGGGSGGGGSGGGGSGGGGSIPPHVQKSVNNDMIVTDNNGA |
| | | VKFPQLCKFCDVRFSTCDNQKSCMSNCSITSICEKPQEVCVAVWRKNDE |
| | | NITLETVCHDPKLPYHDFILEDAASPKCIMKEKKKPGETFFMCSCSSDEC |
| | | NDNIIFSEEYNTSNPD |
| SEQ ID | aCCR2 Hum1 | GAAGTGCAGCTGGTTGAATCTGGCGGCGGACTGGTTAAGCCTGGCGG |
| NO: 132 | VH-hCH1- | ATCTCTGAGACTGTCTTGTGCCGCCTCCGGCTTCTCCTTCAATGCCTAC |
| | hFc_Hole- | GCCATGAACTGGGTCCGACAGGCTCCTGGCAAAGGCCTGGAATGGGT |
| | 4x4GS- | CGGAAGAATCCGGACCAAGAACAACAACTACGCCACCTACTACGCCG |
| | TGFbR2 nt | ACTCCGTGAAGGACCGGTTCACCATCTCTCGGGACGACTCCAAGAGC |
| | | ACCCTGTACCTGCAGATGAACTCCCTGAAAACCGAGGACACCGCCGT |
| | | GTACTACTGCACCACCTTCTACGGCAATGGCGTGTGGGGCCAGGGCA |
| | | CAACAGTGACAGTCTCTTCCGCCTCCACCAAGGGACCCTCTGTGTTTC |
| | | CTCTGGCTCCCTCCAGCAAGTCTACCTCTGGTGGAACAGCTGCCCTGG |
| | | GCTGCCTGGTCAAGGATTACTTTCCTGAGCCTGTGACCGTGTCCTGGA |
| | | ACTCTGGCGCTCTGACATCTGGCGTGAGACCTTTCCAGCTGTGCTGC |
| | | AGTCCTCCGGCCTGTACTCTCTGTCCTCTGTCGTGACCGTGCCTTCCA |
| | | GCTCTCTGGGCACCCAGACCTACATCTGCAATGTGAACCACAAGCCTT |
| | | CCAACACCAAGGTGGACAAGAGAGTGGAACCCAAGTCCTGCGACAA |
| | | GACCCACACCTGTCCTCCATGTCCTGCTCCAGAACTGCTCGGCGGACC |
| | | TTCCGTGTTCCTGTTTCCTCCAAAGCCTAAGGACACCCTGATGATCTC |
| | | TCGGACCCCTGAAGTGACCTGCGTGGTGGTGGATGTGTCTCACGAGG |
| | | ATCCCGAAGTGAAGTTCAATTGGTACGTGGACGGCGTGGAAGTGCAC |
| | | AACGCCAAGACCAAGCCTAGAGAGGAACAGTACAACTCCACCTACA |
| | | GAGTGGTGTCCGTGCTGACCGTGCTGCACCAGGATTGGCTGAACGGC |
| | | AAAGAGTACAAGTGCAAGGTGTCCAACAAGGCCCTGCCTGCTCCTAT |
| | | CGAAAAGACCATCTCCAAGGCCAAGGGCCAGCCTCGGGAACCTCAAG |
| | | TCTGTACCCTGCCTCCTACTCCGGGAAGAGATGACCAAGAATCAGGTG |
| | | TCCCTGTCCTGCGCCGTGAAGGGCTTCTACCCTTCTGATATCGCCGTG |
| | | GAATGGGAGAGCAACGGCCAGCCTGAGAACAACTACAAGACAACCC |
| | | CTCCTGTGCTGGACTCCGACGGCTCATTCTTCCTGGTGTCCAAGCTGA |
| | | CAGTGGACAAGTCCAGATGGCAGCAGGGCAACGTGTTCTCCTGCTCC |
| | | GTGATGCACGAGGCCCTGCACAATCACTACACCCAGAAGTCCCTGTC |
| | | TCTGAGCCCAGGCAAAGGTGGCGGAGGATCTGGCGGAGGTGGTAGC |
| | | GGCGGAGGCGGATCAGGCGGTGGTGGCTCTATTCCTCCACACGTGCA |
| | | GAAATCCGTGAACAACGACATGATCGTGACCGACAACAACGGCGCTG |
| | | TGAAGTTCCCTCAGCTGTGCAAGTTCTGCGACGTGCGGTTCTCCACCT |
| | | GTGACAACCAGAAATCCTGCATGTCCAACTGCTCCATCACCTCCATCT |
| | | GCGAGAAGCCCCAAGAAGTGTGCGTCGCCGTCTGGCGGAAGAACGA |
| | | CGAGAACATCACCCTGGAAACCGTGTGCCACGATCCTAAGCTGCCCT |
| | | ACCACGACTTCATCCTGGAAGATGCCGCCTCTCCTAAGTGCATCATGA |
| | | AGGAAAAGAAGAAGCCCGGCGAGACTTTCTTCATGTGCAGCTGCTCC |
| | | TCCGACGAGTGCAACGACAACATCATCTTCTCCGAAGAGTATAACAC |
| | | CAGCAATCCCGAC |
| SEQ ID | aCCR2 VL | KSSQSLLDSDGKTFLN |
| NO: 454 | LCDR1 | |
| SEQ ID | aCCR2 VL | LVSKLDS |
| NO: 455 | LCDR2 | |
| SEQ ID | aCCR2 VL | WQGTHFPYT |
| NO: 456 | LCDR3 | |
| SEQ ID | aCCR2 VL aa | DVVMTQSPLSLPVTLGQPASISCKSSQSLLDSDGKTFLNWLQQRPGQSPR |
| NO: 345 | | RLIYLVSKLDSGVPDRFSGSGSGTDFTLKISRVEAEDVGVYYCWQGTHFP |
| | | YTFGGGTKVEIK |
| SEQ ID | aCCR2 VL nt | GACGTCGTCATGACCCAATCCCCGCTCTCCCTGCCGGTGACTCTCGGC |
| NO: 272 | | CAGCCGGCGTCCATCTCGTGCAAGTCCTCCCAATCCCTGCTGGATTCG |
| | | GATGGGAAGACTTTCCTGAACTGGCTGCAGCAACGGCCCGGCCAGTC |
| | | GCCACGGCGGCTGATCTACCTGGTGTCCAAGCTGGATTCAGGTGTGC |
| | | CTGATAGATTCTCCGGCAGCGGATCGGGGACCGACTTCACGCTGAAG |
| | | ATCTCCAGAGTGGAAGCAGAGGACGTGGGCGTCTATTACTGTTGGCA |
| | | GGGGACTCATTTCCCCTATACCTTTGGAGGTGGTACTAAGGTGGAAAT |
| | | TAAG |
| SEQ ID | aCCR2 VL- | DVVMTQSPLSLPVTLGQPASISCKSSQSLLDSDGKTFLNWLQQRPGQSPR |
| NO: 373 | hCLIg_vk aa | RLIYLVSKLDSGVPDRFSGSGSGTDFTLKISRVEAEDVGVYYCWQTHFP |
| | | YTFGGGTKVEIKFTVAAPSVFIFPPSDEQLKSGTASVVCLLNNFYPREAK |
| | | VQWKVDNALQSGNSQESVTEQDSKDSTYSLSSTLTLSKADYEKHKVYA |
| | | CEVTHQGLSSPVTKSFNRGEC |
| SEQ ID | aCCR2 VL- | GACGTCGTCATGACCCAATCCCCGCTCTCCCTGCCGGTGACTCTCGGC |
| NO: 134 | hCLIg_vk nt | CAGCCGGCGTCCATCTCGTGCAAGTCCTCCCAATCCCTGCTGGATTCG |
| | | GATGGGAAGACTTTCCTGAACTGGCTGCAGCAACGGCCCGGCCAGTC |
| | | GCCACGGCGGCTGATCTACCTGGTGTCCAAGCTGGATTCAGGTGTGC |
| | | CTGATAGATTCTCCGGCAGCGGATCGGGGACCGACTTCACGCTGAAG |
| | | ATCTCCAGAGTGGAAGCAGAGGACGTGGGCGTCTATTACTGTTGGCA |
| | | GGGGACTCATTTCCCCTATACCTTTGGAGGTGGTACTAAGGTGGAAAT |
| | | TAAGAGAACAGTGGCCGCTCCTTCCGTGTTCATCTTCCCACCCTCCGA |
| | | CGAGCAGCTGAAGTCCGGCACCGCTTCTGTCGTGTGCCTGCTCAACA |
| | | ACTTCTACCCTCGGGAAGCCAAGGTGCAGTGGAAGGTGGACAACGCC |
| | | CTGCAGTCCGGCAACTCCCAGGAATCCGTCACCGAGCAGGACTCCAA |
| | | GGACAGCACCTACTCCCTGTCCTCCACCCTGACCCTGTCCAAGGCCGA |
| | | CTACGAGAAGCACAAGGTGTACGCCTGCGAAGTGACCCACCAGGGCC |
| | | TGAGCAGCCCCGTGACCAAGTCCTTCAACCGGGGCGAGTGC |

### Additional Multispecific Antibody Molecules Targeting CSF1R

In one aspect, disclosed herein is a multispecific antibody molecule comprising a CSF1R binding moiety. In some embodiments, the CSF1R binding moiety comprises an anti-CSF1R antibody molecule. Exemplary anti-CSF1R antibody molecule sequences are described in WO2009026303A1; WO2011123381A1; WO2016207312A1; WO2016106180A1; US20160220669A1; US20160326254A1; WO2013169264A1; WO2013087699A1; WO2011140249A2; WO2011131407A1; WO2011123381A1; WO2011107553A1; and WO2011070024A1, all of which are herein incorporated by reference in their entirety. In some embodiments, the CSF1R binding moiety comprises the CDR (e.g., one, two, three, four, five, or all six CDRs), VH, VL, heavy chain, or light chain sequences of emactuzumab, or a sequence substantially identical thereto (e.g., at least 95% identical thereto, e.g., 95% to 99.9% identical thereto, or having at least one amino acid alteration, but not more than five, ten or fifteen alterations (e.g., substitutions, deletions, or insertions, e.g., conservative substitutions)). In some embodiments, the CSF1R binding moiety comprises the CDR (e.g., one, two, three, four, five, or all six CDRs), VH, VL, heavy chain, or light chain sequences of cabiralizumab, or a sequence substantially identical thereto (e.g., at least 95% identical thereto, e.g., 95% to 99.9% identical thereto, or having at least one amino acid alteration, but not more than five, ten or fifteen alterations (e.g., substitutions, deletions, or insertions, e.g., conservative substitutions)). In some embodiments, the CSF1R binding moiety comprises the CDR (e.g., one, two, three, four, five, or all six CDRs), VH, VL, heavy chain, or light chain sequences of AMG820, or a sequence substantially identical thereto (e.g., at least 95% identical thereto, e.g., 95% to 99.9% identical thereto, or having at least one amino acid alteration, but not more than five, ten or fifteen alterations (e.g., substitutions, deletions, or insertions, e.g., conservative substitutions)). In some embodiments, the CSF1R binding moiety comprises the CDR (e.g., one, two, three, four, five, or all six CDRs), VH, VL, heavy chain, or light chain sequences of IMC-CS4, or a sequence substantially identical thereto (e.g., at least 95% identical thereto, e.g., 95% to 99.9% identical thereto, or having at least one amino acid alteration, but not more than five, ten or fifteen alterations (e.g., substitutions, deletions, or insertions, e.g., conservative substitutions)). In some embodiments, the CSF1R binding moiety comprises a VH or VL amino acid sequence disclosed in Table 8, a CDR of a VH or VL amino acid sequence disclosed in Table 8, or a sequence substantially identical thereto.

TABLE 8
Exemplary anti-CSF1R antibody molecule sequences

| SEQ ID | | |
|---|---|---|
| NO | Description | Sequence |
| SEQ ID | αmCSF1R VH | QVQLQQSGAELVKPGSSVKISCKASGYTFTSNFMHWIKQQPGNGLEWIG |
| NO: 48 | | WIYPGDGDTEYNQKFNGKATLTADKSSSTAYMQLSSLTSEDSAVYFCA |
| | | VNYGGYVLDAWGQGASVTVSS |
| SEQ ID | αmCSF1R VL | EIVLTQSPTTMAASPGEKVTITCRASSSTNYMSWYQQKSGASPKPWIYET |
| NO: 50 | | SKLASGVPDRFSGSGSGTSYSFTISSME1EDAATYYCHQWSSTPLTFGSG |
| | | TKLEIK |
| SEQ ID | αhCSF1R | QVQLVQSGAEVKKPGASVKVSCKASGYTFTSYDISWVRQAPGQGLEW |
| NO: 66 | emactuzumab | MGVIWTDGGTNYAQKLQGRVTMTTDTSTSTAYMELRSLRSDDTAVYY |
| | VH | CARDQRLYFDVWGQGTTVTVSS |
| SEQ ID | αhCSF1R | DIQMTQSPSSLSASVGDRVTITCRASEDVNTYVSWYQQKPGKAPKLLIY |
| NO: 67 | emactuzumab | AASNRYTGVPSRFSGSGSGTDFTLTISSLQPEDFATYYCQQSFSYPTFGQG |
| | VL | TKLEIK |
| SEQ ID | αhCSF1R | QVQLVQSGAEVKKPGSSVKVSCKASGYTFTDNYMIWVRQAPGQGLEW |
| NO: 69 | cabiralizumab | MGDINPYNGGTTFNQKFKGRVTITADKSTSTAYMELSSLRSEDTAVYYC |
| | VH | ARESPYFSNLYVMDYWGQGTLVTVSS |
| SEQ ID | αhCSF1R | EIVLTQSPATLSLSPGERATLSCKASQSVDYDGDNYMNWYQQKPGQAP |
| NO: 70 | cabiralizumab | RLLIYAASNLESGIPARFSGSGSGTDFTLTISSLEPEDFAVYYCHLSNEDLS |
| | VL | TFGGGTKVEIK |

### Additional Multispecific Antibody Molecules Targeting CCR2

In one aspect, disclosed herein is a multispecific antibody molecule comprising a CCR2 binding moiety. Exemplary CCR2 antibodies are described herein as well as in WO2013192596A2; WO2010021697A2; WO2001057226A1; and WO1997031949A1, all of which are herein incorporated by reference in their entirety. In some embodiments, the CCR2 binding moiety comprises the CDR (e.g., one, two, three, four, five, or all six CDRs), VH, VL, heavy chain, or light chain sequences of plozalizumab, or a sequence substantially identical thereto (e.g., 950% to 99.900 identical thereto, or having at least one amino acid alteration, but not more than five, ten or fifteen alterations (e.g., substitutions, deletions, or insertions, e.g., conservative substitutions)). In some embodiments, the CCR2 binding moiety comprises a VH or VL amino acid sequence disclosed in Table 9, a CDR of a VH or VL amino acid sequence disclosed in Table 9, or a sequence substantially identical thereto.

TABLE 9
Exemplary anti-CCR2 antibody molecule sequences

| SEQ ID | | |
|---|---|---|
| NO | Description | Sequence |
| SEQ ID | αCCR2 MC12 | QVQLQESGPGLVQPSQTLSLTCTVSGFSLTDFSVHWVRQPPGKGLEWM |
| NO: 44 | VH | GRIRSEGNTDYNSALKSRLSISRDTSKSQVFLKMNSLQTEDTAIYFCTRG |
| | | DILGFGYWGQGVMVTVSS |
| SEQ ID | αCCR2 MC12 | DIVMTQSPLSVSVTPGESASISCRSSKSLLHFKGITFVYWYLQKPGQSPQL |
| NO: 45 | VL | LIFRMSSLASGVPDRFSGSGSETDFTLKISRVEAEDVGTYYCGQLLENPY |
| | | TFGAGTKLELK |
| SEQ ID | αhCCR2 | EVQLVESGGGLVKPGGSLRLSCAASGFTFSAYAMNWVRQAPGKGLEW |
| NO: 54 | plozalizumab | VGRIRTKNNNYATYYADSVKDRFTISRDDSKNTLYLQMNSLK1EDTAV |
| | VH | YYCTTFYGNGVWGQGTLVTVSS |
| SEQ ID | αhCCR2 | DVVMTQSPLSLPVTLGQPASISCKSSQSLLDSDGKTFLNWFQQRPGQSPR |
| NO: 57 | plozalizumab | RLIYLVSKLDSGVPDRFSGSGSGTDFTLKISRVEAEDVGVYYCWQGTHFP |
| | VL | YTFGQGTRLEIK |
| SEQ ID | αhCCR2 D1 | EVQLVQSGAEVKKPGASVKVSCKASGYTFTGYHMHWVRQAPGQGLEW |
| NO: 59 | VH | MGWINPNSGVTKYAQKFQGRVTMTRDTSINTAYMELSRLRFDDTDVYY |
| | | CATGGFGYWGEGTLVTVSS |
| SEQ ID | αhCCR2 D1 | LPVLTQPPSVSKGLRQTATLTCTGNSNNVGNQGAAWLQQHQGQPPKLL |
| NO: 60 | VL | SYRNHNRPSGVSERFSPSRSGDTSSLTITGLQPEDEADYYCLAWDSSLRA |
| | | FVFGTGTKLTVL |
| SEQ ID | αhCCR2 42G7 | QVQLVQSGAEVKKPGASVKVSCKASGYTFSSYYMHWVRQAPGQGLEW |
| NO: 62 | VH | MGIINPSGGNTSYAQKFQGRVTMTRDTSTSTVYMELSSLRSEDTAVYYC |
| | | ARGGYQLPHGRARAFDMWGQGTMVTVSS |
| SEQ ID | αhCCR2 42G7 | AIRMTQSPLSLPVTLGQPASISCTSSQSLVYRDGTTYLNWFQQRPGQSPR |
| NO: 63 | VL | RLIYKVSNRDSGVPDRFTGSGSGTTFTLTISRVEAEDVGIYYCMQGTHWP |
| | | LTFGQGTKVEIK |
| SEQ ID | αhCCR2 43G12 | EVQLVESGGGLVQPGGSLRLSCVASGFTFSDYWMSWVRQAPGKGLEW |
| NO: 64 | VH | VANIKKDGSVNYYVDSVKGRFTISRDNAKNSLYLQMNSLRAEDTAVYY |
| | | CTRFDYWGQGTLVTVSS |
| SEQ ID | αhCCR2 43G12 | QAGLTQPPSVSKGLRQTATLTCTGNSNNVGNQGAAWLQQHQGHPPKLL |
| NO: 65 | VL | FYRNNNRASGISERLSASRSGNTASLTITGLQPEDEADYYCLTWDSSLSV |
| | | VVFGGGTKLTVL |

### Additional Multispecific Antibody Molecules Targeting PD-L1

In one aspect, disclosed herein is a multispecific antibody molecule comprising a PD-L1 binding moiety. In some embodiments, the PD-L1 binding moiety comprises an anti-PD-L1 antibody molecule. Exemplary anti-PD-L1 antibody molecule sequences are described in WO2013079174, WO 2010077634, WO2007/005874, and US20120039906, all of which are herein incorporated by reference in their entirety. In some embodiments, the PD-L1 binding moiety comprises the CDR (e.g., one, two, three, four, five, or all six CDRs), VH, VL, heavy chain, or light chain sequences of durvalumab, or a sequence substantially identical thereto (e.g., 950% to 99.900 identical thereto, or having at least one amino acid alteration, but not more than five, ten or fifteen alterations (e.g., substitutions, deletions, or insertions, e.g., conservative substitutions)). In some embodiments, the PD-L1 binding moiety comprises the CDR (e.g., one, two, three, four, five, or all six CDRs), VH, VL, heavy chain, or light chain sequences of atezolizumab, or a sequence substantially identical thereto (e.g., at least 950% identical thereto, e.g., 9500 to 99.900 identical thereto, or having at least one amino acid alteration, but not more than five, ten or fifteen alterations (e.g., substitutions, deletions, or insertions, e.g., conservative substitutions)). In some embodiments, the PD-L1 binding moiety comprises the CDR (e.g., one, two, three, four, five, or all six CDRs), VH, VL, heavy chain, or light chain sequences of avelumab, or a sequence substantially identical thereto (e.g., at least 95% identical thereto, e.g., 95% to 99.9% identical thereto, or having at least one amino acid alteration, but not more than five, ten or fifteen alterations (e.g., substitutions, deletions, or insertions, e.g., conservative substitutions)). In some embodiments, the PD-L1 binding moiety comprises a VH or VL amino acid sequence disclosed in Table 10, a CDR of a VH or VL amino acid sequence disclosed in Table 10, or a sequence substantially identical thereto.

TABLE 10
Exemplary anti-PD-L1 antibody molecule sequences

| SEQ ID | | |
|---|---|---|
| NO | Description | Sequence |
| SEQ ID | αPD-L1 | EVQLVESGGGLVQPGGSLRLSCAASGFTFSRYWMSWVRQAPGKGLEW |
| NO: 109 | durvalumab VH | VANIKQDGSEKYYVDSVKGRFTISRDNAKNSLYLQMNSLRAEDTAVY |
| | | YCAREGGWFGELAFDYWGQGTLVTVSS |
| SEQ ID | αPD-L1 | EIVLTQSPGTLSLSPGERATLSCRASQRVSSSYLAWYQQKPGQAPRLLIY |
| NO: 110 | durvalumab VL | DASSRATGIPDRFSGSGSGTDFTLTISRLEPEDFAVYYCQQYGSLPWTFG |
| | | QGTKVEIK |
| SEQ ID | αPD-L1 | EVQLVESGGGLVQPGGSLRLSCAASGFTFSDSWIHWVRQAPGKGLEW |
| NO: 111 | atezolizumab | VAWISPYGGSTYYADSVKGRFTISADTSKNTAYLQMNSLRAEDTAVYY |
| | VH | CARRHWPGGFDYWGQGTLVTVSS |
| SEQ ID | αPD-L1 | DIQMTQSPSSLSASVGDRVTITCRASQDVSTAVAWYQQKPGKAPKLLIY |
| NO: 112 | atezolizumab | SASFLYSGVPSRFSGSGSGTDFTLTISSLQPEDFATYYCQQYLYHPATFG |
| | VL | QGTKVEIK |
| SEQ ID | αPD-L1 | EVQLLESGGGLVQPGGSLRLSCAASGFTFSSYIMMWVRQAPGKGLEW |
| NO: 113 | avelumab VH | VSSIYPSGGITFYADTVKGRFTISRDNSKNTLYLQMNSLRAEDTAVYYC |
| | | ARIKLGTVTTVDYWGQGTLVTVSS |
| SEQ ID | αPD-L1 | QSALTQPASVSGSPGQSITISCTGTSSDVGGYNYVSWYQQHPGKAPKL |
| NO: 114 | avelumab VL | MIYDVSNRPSGVSNRFSGSKSGNTASLTISGLQAEDEADYYCSSYTSSST |
| | | RVFGTGTKVTVL |

### CDR-Grafted Scaffolds

In embodiments, the antibody molecule is a CDR-grafted scaffold domain. In embodiments, the scaffold domain is based on a fibronectin domain, e.g., fibronectin type III domain. The overall fold of the fibronectin type III (Fn3) domain is closely related to that of the smallest functional antibody fragment, the variable region of the antibody heavy chain. There are three loops at the end of Fn3; the positions of BC, DE and FG loops approximately correspond to those of CDR1, 2 and 3 of the VH domain of an antibody. Fn3 does not have disulfide bonds; and therefore Fn3 is stable under reducing conditions, unlike antibodies and their fragments (see, e.g., WO 98/56915; WO 01/64942; WO 00/34784). An Fn3 domain can be modified (e.g., using CDRs or hypervariable loops described herein) or varied, e.g., to select domains that bind to an antigen/marker/cell described herein.

In embodiments, a scaffold domain, e.g., a folded domain, is based on an antibody, e.g., a “minibody” scaffold created by deleting three beta strands from a heavy chain variable region of a monoclonal antibody (see, e.g., Tramontano et al., 1994, J Mol. Recognit. 7:9; and Martin et al., 1994, EMBO J. 13:5303-5309). The “minibody” can be used to present two hypervariable loops. In embodiments, the scaffold domain is a V-like domain (see, e.g., Coia et al. WO 99/45110) or a domain derived from tendamistatin, which is a 74 residue, six-strand beta sheet sandwich held together by two disulfide bonds (see, e.g., McConnell and Hoess, 1995, J Mol. Biol. 250:460). For example, the loops of tendamistatin can be modified (e.g., using CDRs or hypervariable loops) or varied, e.g., to select domains that bind to a marker/antigen/cell described herein. Another exemplary scaffold domain is a beta-sandwich structure derived from the extracellular domain of CTLA-4 (see, e.g., WO 00/60070).

Other exemplary scaffold domains include but are not limited to T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains). See, e.g., US 20040009530 and U.S. Pat. No. 7,501,121, incorporated herein by reference.

In embodiments, a scaffold domain is evaluated and chosen, e.g., by one or more of the following criteria: (1) amino acid sequence, (2) sequences of several homologous domains, (3) 3-dimensional structure, and/or (4) stability data over a range of pH, temperature, salinity, organic solvent, oxidant concentration. In embodiments, the scaffold domain is a small, stable protein domain, e.g., a protein of less than 100, 70, 50, 40 or 30 amino acids. The domain may include one or more disulfide bonds or may chelate a metal, e.g., zinc.

Exemplary structures of the multifunctional molecules defined herein are described below. Exemplary structures are further described in: Weidle U et al. (2013) The Intriguing Options of Multispecific Antibody Formats for Treatment of Cancer. Cancer Genomics & Proteomics 10: 1-18 (2013); and Spiess C et al. (2015) Alternative molecular formats and therapeutic applications for bispecific antibodies. Molecular Immunology 67: 95-106; the full contents of each of which is incorporated by reference herein).

### Heterodimerized Antibody Molecules

Heterodimerized bispecific antibodies are based on the natural IgG structure, wherein the two binding arms recognize different antigens. IgG derived formats that enable defined monovalent (and simultaneous) antigen binding are generated by forced heavy chain heterodimerization, combined with technologies that minimize light chain mispairing (e.g., common light chain). Forced heavy chain heterodimerization can be obtained using, e.g., knob-in-hole OR strand exchange engineered domains (SEED).

### Knob-In-Hole

Knob-in-Hole as described in U.S. Pat. Nos. 5,731,116, 7,476,724 and Ridgway, J. et al. (1996) Prot. Engineering 9(7): 617-621, broadly involves: (1) mutating the CH3 domain of one or both antibodies to promote heterodimerization; and (2) combining the mutated antibodies under conditions that promote heterodimerization. “Knobs” or “protuberances” are typically created by replacing a small amino acid in a parental antibody with a larger amino acid (e.g., T366Y or T366W); “Holes” or “cavities” are created by replacing a larger residue in a parental antibody with a smaller amino acid (e.g., Y407T, T366S, L368A and/or Y407V), numbered based on the Eu numbering system.

### Strand Exchange Engineered Domains (SEED)

SEED is based on sequence exchanges between IgG1 and IgA to create non-identical chains which heterodimerize preferentially. Alternating sequences from human IgA and IgG in the SEED CH3 domains generate two asymmetric but complementary domains, designated AG and GA. The SEED design allows efficient generation of AG/GA heterodimers, while disfavoring homodimerization of AG and GA SEED CH3 domains.

### Common Light Chain & CrossMab

Light chain mispairing must be avoided to generate homogenous preparations of bispecific IgGs. One way to achieve this is through the use of the common light chain principle, i.e. combining two binders that share one light chain but still have separate specificities. Another option is the CrossMab technology which avoids non-specific L chain mispairing by exchanging CH1 and CL domains in the Fab of one half of the bispecific antibody. Such crossover variants retain binding specificity and affinity, but make the two arms so different that L chain mispairing is prevented.

### Antibody-Based Fusions

A variety of formats can be generated which contain additional binding entities attached to the N or C terminus of antibodies. These fusions with single chain or disulfide stabilized Fvs or Fabs result in the generation of tetravalent molecules with bivalent binding specificity for each antigen. Combinations of scFvs and scFabs with IgGs enable the production of molecules which can recognize three or more different antigens.

### Antibody-Fab Fusion

Antibody-Fab fusions are bispecific antibodies comprising a traditional antibody to a first target and a Fab to a second target fused to the C terminus of the antibody heavy chain. Commonly the antibody and the Fab will have a common light chain. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Antibody-scFv Fusion

Antibody-scFv Fusions are bispecific antibodies comprising a traditional antibody and a scFv of unique specificity fused to the C terminus of the antibody heavy chain. The scFv can be fused to the C terminus through the Heavy Chain of the scFv either directly or through a linker peptide. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Variable Domain Immunoglobulin DVD

A related format is the dual variable domain immunoglobulin (DVD), which are composed of VH and VL domains of a second specificity place upon the N termini of the V domains by shorter linker sequences.

### Fc-Containing Entities (Mini-Antibodies)

Fc-containing entities, also known as mini-antibodies, can be generated by fusing scFv to the C-termini of constant heavy region domain 3 (CH3-scFv) and/or to the hinge region (scFv-hinge-Fc) of an antibody with a different specificity. Trivalent entities can also be made which have disulfide stabilized variable regions (without peptide linker) fused to the C-terminus of CH3 domains of IgGs.

### Fc-Less Bispecifics

Fc-less bispecifics are characterized by generally having smaller size than Fc-containing entities. Common bispecific of this class include Fab-scFv2 and Fab-scFv molecules. This class also includes, e.g., BiTEs (bispecific T-cell engagers), diabodies, TandAbs (tetravalent tandem antibodies), and DARTs (dual affinity retargeting molecules). BiTEs are created by fusing two scFvs via a flexible linker peptide. Diabodies consist of two VH and two VL domains from two different antibodies. Interaction only with complementary domains on another chain is achieved by attaching domains with short linker peptides which permits pairing only with VH and VL domains. VH of the first binder linked to the VL of the second binder is co-expressed with the VH of the second antibody linked to VL of the first antibody. TandAbs molecules are generated by functional dimerization of a protein consisting of four antibody variable H- and L-chains in an orientation that prevents intramolecular pairing. DARTs are entities that are stabilized by disulfide bonds which apply a similar design concept to that of diabodies.

### Kappa/Lambda Formats

Multispecific molecules (e.g., multispecific antibody molecules) that include the lambda light chain polypeptide and a kappa light chain polypeptides, can be used to allow for heterodimerization. Methods for generating bispecific antibody molecules comprising the lambda light chain polypeptide and a kappa light chain polypeptides are disclosed in PCT/US2017/53053 filed on Sep. 22, 2017, incorporated herein by reference in its entirety.

In embodiments, the multispecific molecules include a multispecific antibody molecule, e.g., an antibody molecule comprising two binding specificities, e.g., a bispecific antibody molecule. The multispecific antibody molecule includes:

a lambda light chain polypeptide 1 (LLCP1) specific for a first epitope;

a heavy chain polypeptide 1 (HCP1) specific for the first epitope;

a kappa light chain polypeptide 2 (KLCP2) specific for a second epitope; and

a heavy chain polypeptide 2 (HCP2) specific for the second epitope.

“Lambda light chain polypeptide 1 (LLCP1)”, as that term is used herein, refers to a polypeptide comprising sufficient light chain (LC) sequence, such that when combined with a cognate heavy chain variable region, can mediate specific binding to its epitope and complex with an HCP1. In an embodiment it comprises all or a fragment of a CH1 region. In an embodiment, an LLCP1 comprises LC-CDR1, LC-CDR2, LC-CDR3, FR1, FR2, FR3, FR4, and CH1, or sufficient sequence therefrom to mediate specific binding of its epitope and complex with an HCP1. LLCP1, together with its HCP1, provide specificity for a first epitope (while KLCP2, together with its HCP2, provide specificity for a second epitope). As described elsewhere herein, LLCP1 has a higher affinity for HCP1 than for HCP2.

“Kappa light chain polypeptide 2 (KLCP2)”, as that term is used herein, refers to a polypeptide comprising sufficient light chain (LC) sequence, such that when combined with a cognate heavy chain variable region, can mediate specific binding to its epitope and complex with an HCP2. In an embodiments it comprises all or a fragment of a CH1 region. In an embodiment, a KLCP2 comprises LC-CDR1, LC-CDR2, LC-CDR3, FR1, FR2, FR3, FR4, and CH1, or sufficient sequence therefrom to mediate specific binding of its epitope and complex with an HCP2. KLCP2, together with its HCP2, provide specificity for a second epitope (while LLCP1, together with its HCP1, provide specificity for a first epitope).

“Heavy chain polypeptide 1 (HCP1)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiments it comprises all or a fragment of a CH1 region. In an embodiment, it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an LLCP1, (ii) to complex preferentially, as described herein to LLCP1 as opposed to KLCP2; and (iii) to complex preferentially, as described herein, to an HCP2, as opposed to another molecule of HCP1. HCP1, together with its LLCP1, provide specificity for a first epitope (while KLCP2, together with its HCP2, provide specificity for a second epitope).

“Heavy chain polypeptide 2 (HCP2)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiments it comprises all or a fragment of a CH1 region. In an embodiments it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an KLCP2, (ii) to complex preferentially, as described herein to KLCP2 as opposed to LLCP1; and (iii) to complex preferentially, as described herein, to an HCP1, as opposed to another molecule of HCP2. HCP2, together with its KLCP2, provide specificity for a second epitope (while LLCP1, together with its HCP1, provide specificity for a first epitope).

In some embodiments of the multispecific antibody molecule disclosed herein:

LLCP1 has a higher affinity for HCP1 than for HCP2; and/or

KLCP2 has a higher affinity for HCP2 than for HCP1.

In embodiments, the affinity of LLCP1 for HCP1 is sufficiently greater than its affinity for HCP2, such that under preselected conditions, e.g., in aqueous buffer, e.g., at pH 7, in saline, e.g., at pH 7, or under physiological conditions, at least 75%, 80, 90, 95, 98, 99, 99.5, or 99.9% of the multispecific antibody molecule molecules have a LLCP1 complexed, or interfaced with, a HCP1.

In some embodiments of the multispecific antibody molecule disclosed herein:

the HCP1 has a greater affinity for HCP2, than for a second molecule of HCP1; and/or

the HCP2 has a greater affinity for HCP1, than for a second molecule of HCP2.

In embodiments, the affinity of HCP1 for HCP2 is sufficiently greater than its affinity for a second molecule of HCP1, such that under preselected conditions, e.g., in aqueous buffer, e.g., at pH 7, in saline, e.g., at pH 7, or under physiological conditions, at least 75%, 80, 90, 95, 98, 99 99.5 or 99.9% of the multispecific antibody molecule molecules have a HCP1 complexed, or interfaced with, a HCP2.

In another aspect, disclosed herein is a method for making, or producing, a multispecific antibody molecule. The method includes:

(i) providing a first heavy chain polypeptide (e.g., a heavy chain polypeptide comprising one, two, three or all of a first heavy chain variable region (first VH), a first CH1, a first heavy chain constant region (e.g., a first CH2, a first CH3, or both));

(ii) providing a second heavy chain polypeptide (e.g., a heavy chain polypeptide comprising one, two, three or all of a second heavy chain variable region (second VH), a second CH1, a second heavy chain constant region (e.g., a second CH2, a second CH3, or both));

(iii) providing a lambda chain polypeptide (e.g., a lambda light variable region (VLλ), a lambda light constant chain (VLλ), or both) that preferentially associates with the first heavy chain polypeptide (e.g., the first VH); and

(iv) providing a kappa chain polypeptide (e.g., a lambda light variable region (VLκ), a lambda light constant chain (VLκ), or both) that preferentially associates with the second heavy chain polypeptide (e.g., the second VH),

under conditions where (i)-(iv) associate.

In embodiments, the first and second heavy chain polypeptides form an Fc interface that enhances heterodimerization.

In embodiments, (i)-(iv) (e.g., nucleic acid encoding (i)-(iv)) are introduced in a single cell, e.g., a single mammalian cell, e.g., a CHO cell. In embodiments, (i)-(iv) are expressed in the cell.

In embodiments, (i)-(iv) (e.g., nucleic acid encoding (i)-(iv)) are introduced in different cells, e.g., different mammalian cells, e.g., two or more CHO cell. In embodiments, (i)-(iv) are expressed in the cells.

In one embodiments, the method further comprises purifying a cell-expressed antibody molecule, e.g., using a lambda- and/or kappa-specific purification, e.g., affinity chromatography.

In embodiments, the method further comprises evaluating the cell-expressed multispecific antibody molecule. For example, the purified cell-expressed multispecific antibody molecule can be analyzed by techniques known in the art, include mass spectrometry. In one embodiment, the purified cell-expressed antibody molecule is cleaved, e.g., digested with papain to yield the Fab moieties and evaluated using mass spectrometry.

In embodiments, the method produces correctly paired kappa/lambda multispecific, e.g., bispecific, antibody molecules in a high yield, e.g., at least 75%, 80, 90, 95, 98, 99 99.5 or 99.9 0.

In other embodiments, the multispecific, e.g., a bispecific, antibody molecule that includes:

(i) a first heavy chain polypeptide (HCP1) (e.g., a heavy chain polypeptide comprising one, two, three or all of a first heavy chain variable region (first VH), a first CH1, a first heavy chain constant region (e.g., a first CH2, a first CH3, or both)), e.g., wherein the HCP1 binds to a first epitope;

(ii) a second heavy chain polypeptide (HCP2) (e.g., a heavy chain polypeptide comprising one, two, three or all of a second heavy chain variable region (second VH), a second CH1, a second heavy chain constant region (e.g., a second CH2, a second CH3, or both)), e.g., wherein the HCP2 binds to a second epitope;

(iii) a lambda light chain polypeptide (LLCP1) (e.g., a lambda light variable region (VL1), a lambda light constant chain (VL1), or both) that preferentially associates with the first heavy chain polypeptide (e.g., the first VH), e.g., wherein the LLCP1 binds to a first epitope; and

(iv) a kappa light chain polypeptide (KLCP2) (e.g., a lambda light variable region (VLk), a lambda light constant chain (VLk), or both) that preferentially associates with the second heavy chain polypeptide (e.g., the second VH), e.g., wherein the KLCP2 binds to a second epitope.

In embodiments, the first and second heavy chain polypeptides form an Fe interface that enhances heterodimerization. In embodiments, the multispecific antibody molecule has a first binding specificity that includes a hybrid VL1-CLl heterodimerized to a first heavy chain variable region connected to the Fc constant, CH2-CH3 domain (having a knob modification) and a second binding specificity that includes a hybrid VLk-CLk heterodimerized to a second heavy chain variable region connected to the Fc constant, CH2-CH3 domain (having a hole modification).

### Multispecific Molecules Comprising Non-Contiguous Polypeptides

In one embodiment, the multispecific molecule is not a single polypeptide chain.

In one embodiment, the antibody molecule includes two, complete heavy chains and two, complete light chains. In one embodiment, the multispecific molecules having at least two or at least three non-contiguous polypeptide chains include a first and second heavy chain constant regions (e.g., a first and second Fc region) in at least two non-contiguous polypeptide chains, e.g., as described herein.

In embodiments, the multispecific molecule is a bispecific or bifunctional molecule, wherein the first and second polypeptides (i) and (ii) are non-contiguous, e.g., are two separate polypeptide chains. In some embodiments, the first and second polypeptides (i) and (ii) include a paired amino acid substitution at a position chosen from one or more of 347, 349, 350, 351, 366, 368, 370, 392, 394, 395, 397, 398, 399, 405, 407, or 409, e.g., of the Fc region of human IgG1, numbered based on the Eu numbering system. For example, the first heavy chain constant region (e.g., the first Fc region) can include an amino acid substitution chosen from: T366S, L368A, or Y407V (e.g., corresponding to a cavity or hole), and the second heavy chain constant region (e.g., the second Fc region) includes a T366W (e.g., corresponding to a protuberance or knob), numbered based on the Eu numbering system. In some embodiments, the first and second polypeptides are a first and second member of a heterodimeric first and second Fc region.

In some embodiments, the first polypeptide has the following configuration from N-to-C:

(a) a first portion of a first antigen domain, e.g., a first VH-CH1 of a Fab molecule, that binds to a first antigen, e.g., CSF1R, connected, optionally via a linker to, the first heavy chain constant region (e.g., the CH2 connected to the CH3 region) (e.g., a first Fc region); (b) a first portion of a second antigen domain, e.g., a second VH-CH1 of a Fab molecule, that binds to a second antigen, e.g., CCR2 or CXCR2, connected, optionally via a linker to, the second heavy chain constant region (e.g., the CH2 connected to the CH3 region) (e.g., a first Fc region); (c) the third polypeptide has the following configuration from N-to-C: a second portion of the first antigen domain, e.g., a first VL-CL of the Fab, where the VL is of kappa subtype and binds to the first antigen, e.g., CSF1R (e.g., the same antigen bound by the first VH-CH1); (d) the fourth polypeptide has the following configuration from N-to-C: a second portion of the second antigen domain, e.g. a second VL-CL of the Fab, where the VL is of lambda subtype and binds to a second antigen, e.g., a cancer antigen, e.g., CCR2 or CXCR2 (e.g., the same antigen bound by the second VH-CH1).

In embodiments, the first heavy chain constant region (e.g., the first CH2-CH3 region) includes a protuberance or knob, e.g., as described herein. In embodiments, the second heavy chain constant region (e.g., the second CH2-CH3 region) includes a cavity or hole. In embodiments, the first and second heavy chain constant regions promote heterodimerization of the bispecific molecule.

### TGF-Beta Inhibitor

In one aspect, provided herein is a multispecific antibody molecule comprising a TGF-beta inhibitor. In some embodiments, the TGF-beta inhibitor binds to and inhibits TGF-beta, e.g., reduces the activity of TGF-beta. In some embodiments, the TGF-beta inhibitor inhibits (e.g., reduces the activity of) TGF-beta 1. In some embodiments, the TGF-beta inhibitor inhibits (e.g., reduces the activity of) TGF-beta 2. In some embodiments, the TGF-beta inhibitor inhibits (e.g., reduces the activity of) TGF-beta 3. In some embodiments, the TGF-beta inhibitor inhibits (e.g., reduces the activity of) TGF-beta 1 and TGF-beta 3. In some embodiments, the TGF-beta inhibitor inhibits (e.g., reduces the activity of) TGF-beta 1, TGF-beta 2, and TGF-beta 3.

In some embodiments, the TGF-beta inhibitor comprises a portion of a TGF-beta receptor (e.g., an extracellular domain of a TGF-beta receptor) that is capable of inhibiting (e.g., reducing the activity of) TGF-beta, or functional fragment or variant thereof. In some embodiments, the TGF-beta inhibitor comprises a TGFBR1 polypeptide (e.g., an extracellular domain of TGFBR1 or functional variant thereof). In some embodiments, the TGF-beta inhibitor comprises a TGFBR2 polypeptide (e.g., an extracellular domain of TGFBR2 or functional variant thereof). In some embodiments, the TGF-beta inhibitor comprises a TGFBR3 polypeptide (e.g., an extracellular domain of TGFBR3 or functional variant thereof). In some embodiments, the TGF-beta inhibitor comprises a TGFBR1 polypeptide (e.g., an extracellular domain of TGFBR1 or functional variant thereof) and a TGFBR2 polypeptide (e.g., an extracellular domain of TGFBR2 or functional variant thereof). In some embodiments, the TGF-beta inhibitor comprises a TGFBR1 polypeptide (e.g., an extracellular domain of TGFBR1 or functional variant thereof) and a TGFBR3 polypeptide (e.g., an extracellular domain of TGFBR3 or functional variant thereof). In some embodiments, the TGF-beta inhibitor comprises a TGFBR2 polypeptide (e.g., an extracellular domain of TGFBR2 or functional variant thereof) and a TGFBR3 polypeptide (e.g., an extracellular domain of TGFBR3 or functional variant thereof).

Exemplary TGF-beta receptor polypeptides that can be used as TGF-beta inhibitors have been disclosed in U.S. Pat. Nos. 8,993,524, 9,676,863, 8,658,135, US20150056199, US20070184052, and WO2017037634, all of which are herein incorporated by reference in their entirety.

In some embodiments, the TGF-beta inhibitor comprises an extracellular domain of TGFBR1 or a sequence substantially identical thereto (e.g., a sequence that is at least 80%, 85%, 90%, or 95% identical thereto). In some embodiments, the TGF-beta inhibitor comprises an extracellular domain of SEQ ID NO: 95, or a sequence substantially identical thereto (e.g., a sequence that is at least 80%, 85%, 90%, or 95% identical thereto). In some embodiments, the TGF-beta inhibitor comprises an extracellular domain of SEQ ID NO: 96, or a sequence substantially identical thereto (e.g., a sequence that is at least 80%, 85%, 90%, or 95% identical thereto). In some embodiments, the TGF-beta inhibitor comprises an extracellular domain of SEQ ID NO: 97, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 104, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 105, or a sequence substantially identical thereto (e.g., a sequence that is at least 80%, 85%, 90%, or 95% identical thereto).

In some embodiments, the TGF-beta inhibitor comprises an extracellular domain of TGFBR2 or a sequence substantially identical thereto (e.g., a sequence that is at least 80%, 85%, 90%, or 95% identical thereto). In some embodiments, the TGF-beta inhibitor comprises an extracellular domain of SEQ ID NO: 98, or a sequence substantially identical thereto (e.g., a sequence that is at least 80%, 85%, 90%, or 95% identical thereto). In some embodiments, the TGF-beta inhibitor comprises an extracellular domain of SEQ ID NO: 99, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 100, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 101, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 102, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 103, or a sequence substantially identical thereto (e.g., a sequence that is at least 80%, 85%, 90%, or 95% identical thereto).

In some embodiments, the TGF-beta inhibitor comprises an extracellular domain of TGFBR3 or a sequence substantially identical thereto (e.g., a sequence that is at least 80%, 85%, 90%, or 95% identical thereto). In some embodiments, the TGF-beta inhibitor comprises an extracellular domain of SEQ ID NO: 106, or a sequence substantially identical thereto (e.g., a sequence that is at least 80%, 85%, 90%, or 95% identical thereto). In some embodiments, the TGF-beta inhibitor comprises an extracellular domain of SEQ ID NO: 107, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 108, or a sequence substantially identical thereto (e.g., a sequence that is at least 80%, 85%, 90%, or 95% identical thereto).

In some embodiments, the TGF-beta inhibitor comprises no more than one TGF-beta receptor extracellular domain. In some embodiments, the TGF-beta inhibitor comprises two or more (e.g., two, three, four, five, or more) TGF-beta receptor extracellular domains, linked together, e. g., via a linker.

TABLE 12
Exemplary amino acid sequences of TGF-beta polypeptides or TGF-beta receptor
polypeptides

| SEQ ID NO | Description | Amino acid sequence |
|---|---|---|
| SEQ ID NO: 92 | Immature human | MPPSGLRLLLLLLPLLWLLVLTPGRPAAGLSTCKTIDMELVKR |
| | TGF-beta 1 | KRIEAIRGQILSKLRLASPPSQGEVPPGPLPEAVLALYNSTRDRV |
| | (P01137-1) | AGESAEPEPEPEADYYAKEVTRVLMVETHNEIYDKFKQSTHSI |
| | | YMFFNTSELREAVPEPVLLSRAELRLLRLKLKVEQHVELYQKY |
| | | SNNSWRYLSNRLLAPSDSPEWLSFDVTGVVRQWLSRGGEIEGF |
| | | RLSAHCSCDSRDNTLQVDINGFTTGRRGDLATIHGMNRPFLLL |
| | | MATPLERAQHLQSSRHRRALDTNYCFSTEKNCCVRQLYIDFR |
| | | KDLGWKWIHEPKGYHANFCLGPCPYIWSLDTQYSKVLALYNQ |
| | | HNPGASAAPCCVPQALEPLPIVYYVGRKPKVEQLSNMIVRSCK |
| | | CS |
| SEQ ID NO: | Human TGF-beta 1 | LSTCKTIDMELVKRKRIEAIRGQILSKLRLASPPSQGEVPPGPLP |
| 117 | (P01137-1) | EAVLALYNSTRDRVAGESAEPEPEPEADYYAKEVTRVLMVET |
| | | HNEIYDKFKQSTHSIYMFFNTSELREAVPEPVLLSRAELRLLRL |
| | | KLKVEQHVELYQKYSNNSWRYLSNRLLAPSDSPEWLSFDVTG |
| | | VVRQWLSRGGEIEGFRLSAHCSCDSRDNTLQVDINGFTTGRRG |
| | | DLATIHGMNRPFLLLMATPLERAQHLQSSRHRRALDTNYCFSS |
| | | TEKNCCVRQLYIDFRKDLGWKWIHEPKGYHANFCLGPCPYIW |
| | | SLDTQYSKVLALYNQHNPGASAAPCCVPQALEPLPIVYYVGRK |
| | | PKVEQLSNMIVRSCKCS |
| SEQ ID NO: 93 | Immature human | MHYCVLSAFLILHLVTVALSLSTCSTLDMDQFMRKRIEAIRGQI |
| | TGF-beta 2 | LSKLKLTSPPEDYPEPEEVPPEVISIYNSTRDLLQEKASRRAAAC |
| | (P61812-1) | ERERSDEEYYAKEVYKIDMPPFFPSENAIPPTFYRPYFRIVRFDV |
| | | SAMEKNASNLVKAEFRVFRLQNPKARVPEQRIELYQILKSKDL |
| | | TSPTQRYIDSKVVKTRAEGEWLSFDVTDAVHEWLHHKDRNLG |
| | | FKISLHCPCCTFVPSNNYIIPNKSEELEARFAGIDGTSTYTSGDQ |
| | | KTIKSTRKKNSGKTPHLLLMLLPSYRLESQQTNRRKKRALDAA |
| | | YCFRNVQDNCCLRPLYIDFKRDLGWKWIHEPKGYNANFCAGA |
| | | CPYLWSSDTQHSRVLSLYNTINPEASASPCCVSQDLEPLTILYYI |
| | | GKTPKIEQLSNMIVKSCKCS |
| SEQ ID NO: | Human TGF-beta 2 | LSTCSTLDMDQFMRKRIEAIRGQILSKLKLTSPPEDYPEPEEVPP |
| 118 | (P61812-1) | EVISIYNSTRDLLQEKASRRAAACERERSDEEYYAKEVYKIDMP |
| | | PFFPSENAIPPTFYRPYFRIVRFDVSAMEKNASNLVKAEFRVFRL |
| | | QNPKARVPEQRIELYQILKSKDLTSPTQRYIDSKVVKTRAEGEW |
| | | LSFDVTDAVHEWLHHKDRNLGFKISLHCPCCTFVPSNNYIIPNK |
| | | SEELEARFAGIDGTSTYTSGDQKTIKSTRKKNSGKTPHLLLMLL |
| | | PSYRLESQQTNRRKKRALDAAYCFRNVQDNCCLRPLYIDFKRD |
| | | LGWKWIHEPKGYNANFCAGACPYLWSSDTQHSRVLSLYNTIN |
| | | PEASASPCCVSQDLEPLTILYYIGKTPKIEQLSNMIVKSCKCS |
| SEQ ID NO: 94 | Immature human | MKMHLQRALVVLALLNFATVSLSLSTCTTLDFGHIKKKRVEAI |
| | TGF-beta 3 | RGQILSKLRLTSPPEPTVMTHVPYQVLALYNSTRELLEEMHGE |
| | (P10600-1) | REEGCTQENTESEYYAKEIHKFDMIQGLAEHNELAVCPKGITSK |
| | | VFRFNVSSVEKNRTNLFRAEFRVLRVPNPSSKRNEQRIELFQILR |
| | | PDEHIAKQRYIGGKNLPTRGTAEWLSFDVTDTVREWLLRRESN |
| | | LGLEISIHCPCHTFQPNGDILENIHEVMEIKFKGVDNEDDHGRG |
| | | DLGRLKKQKDHHNPHLILMMIPPHRLDNPGQGGQRKKRALDT |
| | | NYCFRNLEENCCVRPLYIDFRQDLGWKWVHEPKGYYANFCSG |
| | | PCPYLRSADTTHSTVLGLYNTLNPEASASPCCVPQDLEPLTILY |
| | | YVGRTPKVEQLSNMVVKSCKCS |
| SEQ ID NO: | Human TGF-beta 3 | LSTCTTLDFGHIKKKRVEAIRGQILSKLRLTSPPEPTVMTHVPYQ |
| 119 | (P10600-1) | VLALYNSTRELLEEMHGEREEGCTQEN1ESEYYAKEIHKFDMI |
| | | QGLAEHNELAVCPKGITSKVFRFNVSSVEKNRTNLFRAEFRVL |
| | | RVPNPSSKRNEQRIELFQILRPDEHIAKQRYIGGKNLPTRGTAE |
| | | WLSFDVTDTVREWLLRRESNLGLEISIHCPCHTFQPNGDILENIH |
| | | EVMEIKFKGVDNEDDHGRGDLGRLKKQKDHHNPHLILMMIPP |
| | | HRLDNPGQGGQRKKRALDTNYCFRNLEENCCVRPLYIDFRQD |
| | | LGWKWVHEPKGYYANFCSGPCPYLRSADTTHSTVLGLYNTLN |
| | | PEASASPCCVPQDLEPLTILYYVGRTPKVEQLSNMVVKSCKCS |
| SEQ ID NO: 95 | Immature human | MEAAVAAPRPRLLLLVLAAAAAAAAALLPGATALQCFCHLCT |
| | TGFBR1 isoform 1 | KDNFTCVTDGLCFVSVTETTDKVIHNSMCIAEIDLIPRDRPFVC |
| | (P36897-1) | APSSKTGSVTTTYCCNQDHCNKIELPTTVKSSPGLGPVELAAVI |
| | | AGPVCFVCISLMLMVYICHNRTVIHHRVPNEEDPSLDRPFISEG |
| | | TTLKDLIYDMTTSGSGSGLPLLVQRTIARTIVLQESIGKGRFGEV |
| | | WRGKWRGEEVAVKIFSSREERSWFREAEIYQTVMLRHENILGF |
| | | IAADNKDNGTWTQLWLVSDYHEHGSLFDYLNRYTVTVEGMIK |
| | | LALSTASGLAHLHMEIVGTQGKPAIAHRDLKSKNILVKKNGTC |
| | | CIADLGLAVRHDSATDTIDIAPNHRVGTKRYMAPEVLDDSINM |
| | | KHFESFKRADIYAMGLVFWEIARRCSIGGIHEDYQLPYYDLVPS |
| | | DPSVEEMRKVVCEQKLRPNIPNRWQSCEALRVMAKIMRECWY |
| | | ANGAARLTALRIKKTLSQLSQQEGIKM |
| SEQ ID NO: | Human TGFBR1 | LQCFCHLCTKDNFTCVTDGLCFVSVTETTDKVIHNSMCIAEIDL |
| 120 | isoform 1 (P36897- | IPRDRPFVCAPSSKTGSVTTTYCCNQDHCNKIELPTTVKSSPGL |
| | 1) | GPVELAAVIAGPVCFVCISLMLMVYICHNRTVIHHRVPNEEDPS |
| | | LDRPFISEGTTLKDLIYDMTTSGSGSGLPLLVQRTIARTIVLQESI |
| | | GKGRFGEVWRGKWRGEEVAVKIFSSREERSWFREAEIYQTVM |
| | | LRHENILGFIAADNKDNGTWTQLWLVSDYHEHGSLFDYLNRY |
| | | TVTVEGMIKLALSTASGLAHLHMEIVGTQGKPAIAHRDLKSKN |
| | | ILVKKNGTCCIADLGLAVRHDSATDTIDIAPNHRVGTKRYMAP |
| | | EVLDDSINNIKHFESFKRADIYAMGLVFWEIARRCSIGGIHEDYQ |
| | | LPYYDLVPSDPSVEEMRKVVCEQKLRPNIPNRWQSCEALRVM |
| | | AKIMRECWYANGAARLTALRIKKTLSQLSQQEGIKM |
| SEQ ID NO: 96 | Immature human | MEAAVAAPRPRLLLLVLAAAAAAAAALLPGATALQCFCHLCT |
| | TGFBR1 isoform 2 | KDNFTCVTDGLCFVSVTETTDKVIHNSMCIAEIDLIPRDRPFVC |
| | (P36897-2) | APSSKTGSVTTTYCCNQDHCNKIELPTTGPFSVKSSPGLGPVEL |
| | | AAVIAGPVCFVCISLMLMVYICHNRTVIHHRVPNEEDPSLDRPF |
| | | ISEGTTLKDLIYDMTTSGSGSGLPLLVQRTIARTIVLQESIGKGR |
| | | FGEVWRGKWRGEEVAVKIFSSREERSWFREAEIYQTVMLRHE |
| | | NILGFIAADNKDNGTWTQLWLVSDYHEHGSLFDYLNRYTVTV |
| | | EGMIKLALSTASGLAHLHMEIVGTQGKPAIAHRDLKSKNILVK |
| | | KNGTCCIADLGLAVRHDSATDTIDIAPNHRVGTKRYMAPEVLD |
| | | DSINNIKHFESFKRADIYAMGLVFWEIARRCSIGGIHEDYQLPYY |
| | | DLVPSDPSVEEMRKVVCEQKLRPNIPNRWQSCEALRVMAKIM |
| | | RECWYANGAARLTALRIKKTLSQLSQQEGIKM |
| SEQ ID NO: | Human TGFBR1 | LQCFCHLCTKDNFTCVTDGLCFVSVTETTDKVIHNSMCIAEIDL |
| 121 | isoform 2 (P36897- | IPRDRPFVCAPSSKTGSVTTTYCCNQDHCNKIELPTTGPFSVKSS |
| | 2) | PGLGPVELAAVIAGPVCFVCISLMLMVYICHNRTVIHHRVPNEE |
| | | DPSLDRPFISEGTTLKDLIYDMTTSGSGSGLPLLVQRTIARTIVL |
| | | QESIGKGRFGEVWRGKWRGEEVAVKIFSSREERSWFREAEIYQ |
| | | TVMLRHENILGFIAADNKDNGTWTQLWLVSDYHEHGSLFDYL |
| | | NRYTVTVEGMIKLALSTASGLAHLHMEIVGTQGKPAIAHRDLK |
| | | SKNILVKKNGTCCIADLGLAVRHDSATDTIDIAPNHRVGTKRY |
| | | MAPEVLDDSINMKHFESFKRADIYAMGLVFWEIARRCSIGGIHE |
| | | DYQLPYYDLVPSDPSVEEMRKVVCEQKLRPNIPNRWQSCEALR |
| | | VMAKIMRECWYANGAARLTALRIKKTLSQLSQQEGIKM |
| SEQ ID NO: 97 | Immature human | MEAAVAAPRPRLLLLVLAAAAAAAAALLPGATALQCFCHLCT |
| | TGFBR1 isoform 3 | KDNFTCVTDGLCFVSVTETTDKVIHNSMCIAEIDLIPRDRPFVC |
| | (P36897-3) | APSSKTGSVTTTYCCNQDHCNKIELPTTGLPLLVQRTIARTIVLQ |
| | | ESIGKGRFGEVWRGKWRGEEVAVKIFSSREERSWFREAEIYQT |
| | | VMLRHENILGFIAADNKDNGTWTQLWLVSDYHEHGSLFDYLN |
| | | RYTVTVEGMIKLALSTASGLAHLHMEIVGTQGKPAIAHRDLKS |
| | | KNILVKKNGTCCIADLGLAVRHDSATDTIDIAPNHRVGTKRYM |
| | | APEVLDDSINNIKHFESFKRADIYAMGLVFWEIARRCSIGGIHED |
| | | YQLPYYDLVPSDPSVEEMRKVVCEQKLRPNIPNRWQSCEALRV |
| | | MAKIMRECWYANGAARLTALRIKKTLSQLSQQEGIKM |
| SEQ ID NO: | Human TGFBR1 | LQCFCHLCTKDNFTCVTDGLCFVSVTETTDKVIHNSMCIAEIDL |
| 122 | isoform 3 (P36897- | IPRDRPFVCAPSSKTGSVTTTYCCNQDHCNKIELPTTGLPLLVQ |
| | 3) | RTIARTIVLQESIGKGRFGEVWRGKWRGEEVAVKIFSSREERSW |
| | | FREAEIYQTVMLRHENILGFIAADNKDNGTWTQLWLVSDYHE |
| | | HGSLFDYLNRYTVTVEGMIKLALSTASGLAHLHMEIVGTQGKP |
| | | AIAHRDLKSKNILVKKNGTCCIADLGLAVRHDSATDTIDIAPNH |
| | | RVGTKRYMAPEVLDDSINNIKHFESFKRADIYAMGLVFWEIAR |
| | | RCSIGGIHEDYQLPYYDLVPSDPSVEEMRKVVCEQKLRPNIPNR |
| | | WQSCEALRVMAKIMRECWYANGAARLTALRIKKTLSQLSQQE |
| | | GIKM |
| SEQ ID NO: | Human TGFBR1 | LQCFCHLCTKDNFTCVTDGLCFVSVTETTDKVIHNSMCIAEIDL |
| 104 | fragment 1 | IPRDRPFVCAPSSKTGSVTTTYCCNQDHCNKIELPTTVKSSPGL |
| | | GPVEL |
| SEQ ID NO: | Human TGFBR1 | ALQCFCHLCTKDNFTCVTDGLCFVSVTETTDKVIHNSMCIAEID |
| 105 | fragment 2 | LIPRDRPFVCAPSSKTGSVTTTYCCNQDHCNKIEL |
| SEQ ID NO: | TGFβR1 fragment 3 | LLPGATALQCFCHLCTKDNFTCVTDGLCFVSV1ETTDKVIHNS |
| 135 | | MCIAEIDLIPRDRPFVCAPSSKTGSVTTTYCCNQDHCNKIELPTT |
| | | VKSSPGLGPVE |
| SEQ ID NO: 98 | Immature human | MGRGLLRGLWPLHIVLWTRIASTIPPHVQKSVNNDMIVTDNNG |
| | TGFBR2 isoform B | AVKFPQLCKFCDVRFSTCDNQKSCMSNCSITSICEKPQEVCVA |
| | (short isoform) | VWRKNDENITLETVCHDPKLPYHDFILEDAASPKCIMKEKKKP |
| | (P37173-1) | GETFFMCSCSSDECNDNIIFSEEYNTSNPDLLLVIFQVTGISLLPP |
| | | LGVAISVIIIFYCYRVNRQQKLSSTWETGKTRKLMEFSEHCAIIL |
| | | EDDRSDISSTCANNINHNTELLPIELDTLVGKGRFAEVYKAKLK |
| | | QNTSEQFETVAVKIFPYEEYASWKIEKDIFSDINLKHENILQFLT |
| | | AEERKTELGKQYWLITAFHAKGNLQEYLTRHVISWEDLRKLGS |
| | | SLARGIAHLHSDHTPCGRPKMPIVHRDLKSSNILVKNDLTCCLC |
| | | DFGLSLRLDPTLSVDDLANSGQVGTARYMAPEVLESRMNLEN |
| | | VESFKQTDVYSMALVLWEMTSRCNAVGEVKDYEPPFGSKVRE |
| | | HPCVESMKDNVLRDRGRPEIPSFWLNHQGIQMVCETLTECWD |
| | | HDPEARLTAQCVAERFSELEHLDRLSGRSCSEEKIPEDGSLNTT |
| | | K |
| SEQ ID NO: | Human TGFBR2 | TIPPHVQKSVNNDMIVTDNNGAVKFPQLCKFCDVRFSTCDNQK |
| 123 | isoform B (short | SCMSNCSITSICEKPQEVCVAVWRKNDENITLETVCHDPKLPYH |
| | isoform) | DFILEDAASPKCIMKEKKKPGETFFMCSCSSDECNDNIIFSEEYN |
| | (P37173-1) | TSNPDLLLVIFQVTGISLLPPLGVAISVIIIFYCYRVNRQQKLSST |
| | | WETGKTRKLMEFSEHCAIILEDDRSDISSTCANNINHNIELLPIE |
| | | LDTLVGKGRFAEVYKAKLKQNTSEQFETVAVKIFPYEEYASW |
| | | KTEKDIFSDINLKHENILQFLTAEERKTELGKQYWLITAFHAKG |
| | | NLQEYLTRHVISWEDLRKLGSSLARGIAHLHSDHTPCGRPKMPI |
| | | VHRDLKSSNILVKNDLTCCLCDFGLSLRLDPTLSVDDLANSGQ |
| | | VGTARYMAPEVLESRMNLENVESFKQTDVYSMALVLWEMTS |
| | | RCNAVGEVKDYEPPFGSKVREHPCVESMKDNVLRDRGRPEIPS |
| | | FWLNHQGIQMVCETLTECWDHDPEARLTAQCVAERFSELEHL |
| | | DRLSGRSCSEEKIPEDGSLNTTK |
| SEQ ID NO: 99 | Immature human | MGRGLLRGLWPLHIVLWTRIASTIPPHVQKSDVEMEAQKDEIIC |
| | TGFBR2 isoform A | PSCNRTAHPLRHINNDMIVTDNNGAVKFPQLCKFCDVRFSTCD |
| | (long isoform) | NQKSCMSNCSITSICEKPQEVCVAVWRKNDENITLETVCHDPK |
| | (P37173-2) | LPYHDFILEDAASPKCIMKEKKKPGETFFMCSCSSDECNDNIIFS |
| | | EEYNTSNPDLLLVIFQVTGISLLPPLGVAISVIIIFYCYRVNRQQK |
| | | LSSTWETGKTRKLMEFSEHCAIILEDDRSDISSTCANNINHNILL |
| | | LPIELDTLVGKGRFAEVYKAKLKQNTSEQFETVAVKIFPYEEYA |
| | | SWKIEKDIFSDINLKHENILQFLTAEERKTELGKQYWLITAFHA |
| | | KGNLQEYLTRHVISWEDLRKLGSSLARGIAHLHSDHTPCGRPK |
| | | MPIVHRDLKSSNILVKNDLTCCLCDFGLSLRLDPTLSVDDLANS |
| | | GQVGTARYMAPEVLESRMNLENVESFKQTDVYSMALVLWEM |
| | | TSRCNAVGEVKDYEPPFGSKVREHPCVESMKDNVLRDRGRPEI |
| | | PSFWLNHQGIQMVCETLTECWDHDPEARLTAQCVAERFSELE |
| | | HLDRLSGRSCSEEKIPEDGSLNTTK |
| SEQ ID NO: | Human TGFBR2 | TIPPHVQKSDVEMEAQKDEIICPSCNRTAHPLRHINNDMIVTDN |
| 124 | isoform A (long | NGAVKFPQLCKFCDVRFSTCDNQKSCMSNCSITSICEKPQEVC |
| | isoform) | VAVWRKNDENITLETVCHDPKLPYHDFILEDAASPKCIMKEKK |
| | (P37173-2) | KPGETFFMCSCSSDECNDNIIFSEEYNTSNPDLLLVIFQVTGISLL |
| | | PPLGVAISVIIIFYCYRVNRQQKLSSTWETGKTRKLMEFSEHCAI |
| | | ILEDDRSDISSTCANNINHNTELLPIELDTLVGKGRFAEVYKAKL |
| | | KQNTSEQFETVAVKIFPYEEYASWKTEKDIFSDINLKHENILQFL |
| | | TAEERKTELGKQYWLITAFHAKGNLQEYLTRHVISWEDLRKL |
| | | GSSLARGIAHLHSDHTPCGRPKMPIVHRDLKSSNILVKNDLTCC |
| | | LCDFGLSLRLDPTLSVDDLANSGQVGTARYMAPEVLESRMNL |
| | | ENVESFKQTDVYSMALVLWEMTSRCNAVGEVKDYEPPFGSKV |
| | | REHPCVESMKDNVLRDRGRPEIPSFWLNHQGIQMVCETLTEC |
| | | WDHDPEARLTAQCVAERFSELEHLDRLSGRSCSEEKIPEDGSLN |
| | | TTK |
| SEQ ID NO: | Human TGFBR2 | TIPPHVQKSVNNDMIVTDNNGAVKFPQLCKFCDVRFSTCDNQK |
| 100 | fragment 1 (ECD of | SCMSNCSITSICEKPQEVCVAVWRKNDENITLETVCHDPKLPYH |
| | human TGFBR2 | DFILEDAASPKCIMKEKKKPGETFFMCSCSSDECNDNIIFSEEYN |
| | isoform B) | TSNPD |
| SEQ ID NO: | Human TGFBR2 | IPPHVQKSVNNDMIVTDNNGAVKFPQLCKFCDVRFSTCDNQKS |
| 101 | fragment 2 | CMSNCSITSICEKPQEVCVAVWRKNDENITLETVCHDPKLPYH |
| | | DFILEDAASPKCIMKEKKKPGETFFMCSCSSDECNDNIIFSEEYN |
| | | TSNPD |
| SEQ ID NO: | Human TGFBR2 | TIPPHVQKSDVEMEAQKDEIICPSCNRTAHPLRHINNDMIVTDN |
| 102 | fragment 3 (ECD of | NGAVKFPQLCKFCDVRFSTCDNQKSCMSNCSITSICEKPQEVC |
| | human TGFBR2 | VAVWRKNDENITLETVCHDPKLPYHDFILEDAASPKCIMKEKK |
| | isoform A) | KPGETFFMCSCSSDECNDNIIFSEEYNTSNPD |
| SEQ ID NO: | Human TGFBR2 | QLCKFCDVRFSTCDNQKSCMSNCSITSICEKPQEVCVAVWRKN |
| 103 | fragment 4 | DENITLETVCHDPKLPYHDFILEDAASPKCIMKEKKKPGETFFM |
| | | CSCSSDECNDNIIF |
| SEQ ID NO: | Immature human | MTSHYVIAIFALMSSCLATAGPEPGALCELSPVSASHPVQALME |
| 106 | TGFBR3 isoform 1 | SFTVLSGCASRGTTGLPQEVHVLNLRTAGQGPGQLQREVTLHL |
| | (Q03167-1) | NPISSVHIHHKSVVFLLNSPHPLVWHLKTERLATGVSRLFLVSE |
| | | GSVVQFSSANFSLTAETEERNFPHGNEHLLNWARKEYGAVTSF |
| | | TELKIARNIYIKVGEDQVFPPKCNIGKNFLSLNYLAEYLQPKAA |
| | | EGCVMSSQPQNEEVHIIELITPNSNPYSAFQVDITIDIRPSQEDLE |
| | | VVKNLILILKCKKSVNWVIKSFDVKGSLKIIAPNSIGFGKESERS |
| | | MTMTKSIRDDIPSTQGNLVKWALDNGYSPITSYTMAPVANRFH |
| | | LRLENNAEEMGDEEVHTIPPELRILLDPGALPALQNPPIRGGEG |
| | | QNGGLPFPFPDISRRVWNEEGEDGLPRPKDPVIPSIQLFPGLREP |
| | | EEVQGSVDIALSVKCDNEKMIVAVEKDSFQASGYSGMDVTLL |
| | | DPTCKAKMNGTHFVLESPLNGCGTRPRWSALDGVVYYNSIVIQ |
| | | VPALGDSSGWPDGYEDLESGDNGFPGDMDEGDASLFTRPEIVV |
| | | FNCSLQQVRNPSSFQEQPHGNITFNMELYNTDLFLVPSQGVFSV |
| | | PENGHVYVEVSVTKAEQELGFAIQTCFISPYSNPDRMSHYTIIEN |
| | | ICPKDESVKFYSPKRVHFPIPQADMDKKRFSFVFKPVFNTSLLF |
| | | LQCELTLCTKMEKHPQKLPKCVPPDEACTSLDASIIWAMMQN |
| | | KKTFTKPLAVIHHEAESKEKGPSMKEPNPISPPIFHGLDTLTVM |
| | | GIAFAAFVIGALLTGALWYIYSHTGETAGRQQVPTSPPASENSS |
| | | AAHSIGSTQSTPCSSSSTA |
| SEQ ID NO: | Human TGFBR3 | GPEPGALCELSPVSASHPVQALMESFTVLSGCASRGTTGLPQEV |
| 125 | isoform 1 (Q03167- | HVLNLRTAGQGPGQLQREVTLHLNPISSVHIHHKSVVFLLNSPH |
| | 1) | PLVWHLKTERLATGVSRLFLVSEGSVVQFSSANFSLTAETEERN |
| | | FPHGNEHLLNWARKEYGAVTSFTELKIARNIYIKVGEDQVFPP |
| | | KCNIGKNFLSLNYLAEYLQPKAAEGCVMSSQPQNEEVHIIELIT |
| | | PNSNPYSAFQVDITIDIRPSQEDLEVVKNLILILKCKKSVNWVIK |
| | | SFDVKGSLKIIAPNSIGFGKESERSMTMTKSIRDDIPSTQGNLVK |
| | | WALDNGYSPITSYTMAPVANRFHLRLENNAEEMGDEEVHTIPP |
| | | ELRILLDPGALPALQNPPIRGGEGQNGGLPFPFPDISRRVWNEE |
| | | GEDGLPRPKDPVIPSIQLFPGLREPEEVQGSVDIALSVKCDNEK |
| | | MIVAVEKDSFQASGYSGMDVTLLDPTCKAKMNGTHFVLESPL |
| | | NGCGTRPRWSALDGVVYYNSIVIQVPALGDSSGWPDGYEDLE |
| | | SGDNGFPGDMDEGDASLFTRPEIVVFNCSLQQVRNPSSFQEQP |
| | | HGNITFNMELYNTDLFLVPSQGVFSVPENGHVYVEVSVTKAEQ |
| | | ELGFAIQTCFISPYSNPDRMSHYTIIENICPKDESVKFYSPKRVHF |
| | | PIPQADMDKKRFSFVFKPVFNTSLLFLQCELTLCTKMEKHPQKL |
| | | PKCVPPDEACTSLDASIIWAMMQNKKTFTKPLAVIHHEAESKE |
| | | KGPSMKEPNPISPPIFHGLDTLTVMGIAFAAFVIGALLTGALWYI |
| | | YSHTGETAGRQQVPTSPPASENSSAAHSIGSTQSTPCSSSSTA |
| SEQ ID NO: | Immature human | MTSHYVIAIFALMSSCLATAGPEPGALCELSPVSASHPVQALME |
| 107 | TGFBR3 isoform 2 | SFTVLSGCASRGTTGLPQEVHVLNLRTAGQGPGQLQREVTLHL |
| | (Q03167-2) | NPISSVHIHHKSVVFLLNSPHPLVWHLKTERLATGVSRLFLVSE |
| | | GSVVQFSSANFSLTAETEERNFPHGNEHLLNWARKEYGAVTSF |
| | | TELKIARNIYIKVGEDQVFPPKCNIGKNFLSLNYLAEYLQPKAA |
| | | EGCVMSSQPQNEEVHIIELITPNSNPYSAFQVDITIDIRPSQEDLE |
| | | VVKNLILILKCKKSVNWVIKSFDVKGSLKIIAPNSIGFGKESERS |
| | | MTMTKSIRDDIPSTQGNLVKWALDNGYSPITSYTMAPVANRFH |
| | | LRLENNEEMGDEEVHTIPPELRILLDPGALPALQNPPIRGGEGQ |
| | | NGGLPFPFPDISRRVWNEEGEDGLPRPKDPVIPSIQLFPGLREPE |
| | | EVQGSVDIALSVKCDNEKMIVAVEKDSFQASGYSGMDVTLLD |
| | | PTCKAKMNGTHFVLESPLNGCGTRPRWSALDGVVYYNSIVIQV |
| | | PALGDSSGWPDGYEDLESGDNGFPGDMDEGDASLFTRPEIVVF |
| | | NCSLQQVRNPSSFQEQPHGNITFNMELYNTDLFLVPSQGVFSVP |
| | | ENGHVYVEVSVTKAEQELGFAIQTCFISPYSNPDRMSHYTIIENI |
| | | CPKDESVKFYSPKRVHFPIPQADMDKKRFSFVFKPVFNTSLLFL |
| | | QCELTLCTKMEKHPQKLPKCVPPDEACTSLDASIIWAMMQNK |
| | | KTFTKPLAVIHHEAESKEKGPSMKEPNPISPPIFHGLDTLTVMGI |
| | | AFAAFVIGALLTGALWYIYSHTGETAGRQQVPTSPPASENSSAA |
| | | HSIGSTQSTPCSSSSTA |
| SEQ ID NO: | Human TGFBR3 | GPEPGALCELSPVSASHPVQALMESFTVLSGCASRGTTGLPQEV |
| 126 | isoform 2 (Q03167- | HVLNLRTAGQGPGQLQREVTLHLNPISSVHIHHKSVVFLLNSPH |
| | 2) | PLVWHLKTERLATGVSRLFLVSEGSVVQFSSANFSLTAETEERN |
| | | FPHGNEHLLNWARKEYGAVTSFTELKIARNIYIKVGEDQVFPP |
| | | KCNIGKNFLSLNYLAEYLQPKAAEGCVMSSQPQNEEVHIIELIT |
| | | PNSNPYSAFQVDITIDIRPSQEDLEVVKNLILILKCKKSVNWVIK |
| | | SFDVKGSLKIIAPNSIGFGKESERSMTMTKSIRDDIPSTQGNLVK |
| | | WALDNGYSPITSYTMAPVANRFHLRLENNEEMGDEEVHTIPPE |
| | | LRILLDPGALPALQNPPIRGGEGQNGGLPFPFPDISRRVWNEEG |
| | | EDGLPRPKDPVIPSIQLFPGLREPEEVQGSVDIALSVKCDNEKMI |
| | | VAVEKDSFQASGYSGMDVTLLDPTCKAKMNGTHFVLESPLNG |
| | | CGTRPRWSALDGVVYYNSIVIQVPALGDSSGWPDGYEDLESG |
| | | DNGFPGDMDEGDASLFTRPEIVVFNCSLQQVRNPSSFQEQPHG |
| | | NITFNMELYNTDLFLVPSQGVFSVPENGHVYVEVSVTKAEQEL |
| | | GFAIQTCFISPYSNPDRMSHYTIIENICPKDESVKFYSPKRVHFPI |
| | | PQADMDKKRFSFVFKPVFNTSLLFLQCELTLCTKMEKHPQKLP |
| | | KCVPPDEACTSLDASIIWANIMQNKKTFTKPLAVIHHEAESKEK |
| | | GPSMKEPNPISPPIFHGLDTLTVMGIAFAAFVIGALLTGALWYIY |
| | | SHTGETAGRQQVPTSPPASENSSAAHSIGSTQSTPCSSSSTA |
| SEQ ID NO: | Human TGFBR3 | GPEPGALCELSPVSASHPVQALMESFTVLSGCASRGTTGLPQEV |
| 108 | fragment 1 | HVLNLRTAGQGPGQLQREVTLHLNPISSVEIHEKSVVFLLNSPE |
| | | PLVWHLKTERLATGVSRLFLVSEGSVVQFSSANFSLTAETEERN |
| | | FPEGNEELLNWARKEYGAVTSFTELKIARNIYIKVGEDQVFPP |
| | | KCNIGKNFLSLNYLAEYLQPKAAEGCVMSSQPQNEEVHIIELIT |
| | | PNSNPYSAFQVDITIDIRPSQEDLEVVKNLILILKCKKSVNWVIK |
| | | SFDVKGSLKIIAPNSIGFGKESERSMTMTKSIRDDIPSTQGNLVK |
| | | WALDNGYSPITSYTMAPVANRFELRLENNAEEMGDEEVETIPP |
| | | ELRILLDPGALPALQNPPIRGGEGQNGGLPFPFPDISRRVWNEE |
| | | GEDGLPRPKDPVIPSIQLFPGLREPEEVQGSVDIALSVKCDNEK |
| | | MIVAVEKDSFQASGYSGMDVTLLDPTCKAKMNGTHFVLESPL |
| | | NGCGTRPRWSALDGVVYYNSIVIQVPALGDSSGWPDGYEDLE |
| | | SGDNGFPGDMDEGDASLFTRPEIVVFNCSLQQVRNPSSFQEQP |
| | | HGNITFNMELYNTDLFLVPSQGVFSVPENGHVYVEVSVTKAEQ |
| | | ELGFAIQTCFISPYSNPDRMSHYTIIENICPKDESVKFYSPKRVHF |
| | | PIPQADMDKKRFSFVFKPVFNTSLLFLQCELTLCTKMEKHPQKL |
| | | PKCVPPDEACTSLDASIIWAMMQNKKTFTKPLAVIHHEAESKE |
| | | KGPSMKEPNPISPPIFHGLDTLTV |
| SEQ ID NO: | hCH1-hFc Hole- | ASTKGPSVFPLAPSSKSTSGGTAALGCLVKDYFPEPVTVSWNS |
| 192 | 3x4GS-TGFbR2 | GALTSGVHTFPAVLQSSGLYSLSSVVTVPSSSLGTQTYICNVNH |
| | | KPSNTKVDKRVEPKSCDKTHTCPPCPAPELLGGPSVFLFPPKPK |
| | | DTLMISRTPEVTCVVVDVSHEDPEVKFNWYVDGVEVHNAKTK |
| | | PREEQYNSTYRVVSVLTVLHQDWLNGKEYKCKVSNKALPAPI |
| | | EKTISKAKGQPREPQVCTLPPSREEMTKNQVSLSCAVKGFYPSD |
| | | IAVEWESNGQPENNYKTTPPVLDSDGSFFLVSKLTVDKSRWQQ |
| | | GNVFSCSVMHEALHNHYTQKSLSLSPGXGGGGSGGGGSGGGG |
| | | SIPPHVQKSVNNDMIVTDNNGAVKFPQLCKFCDVRFSTCDNQK |
| | | SCMSNCSITSICEKPQEVCVAVWRKNDENITLETVCEDPKLPYE |
| | | DFILEDAASPKCIMKEKKKPGETFFMCSCSSDECNDNIIFSEEYN |
| | | TSNPD, wherein X is K or absent |
| SEQ ID NO: | hCH1-hFc Knob- | ASTKGPSVFPLAPSSKSTSGGTAALGCLVKDYFPEPVTVSWNS |
| 193 | 3x4GS-TGFbR2 | GALTSGVHTFPAVLQSSGLYSLSSVVTVPSSSLGTQTYICNVNH |
| | | KPSNTKVDKRVEPKSCDKTHTCPPCPAPELLGGPSVFLFPPKPK |
| | | DTLMISRTPEVTCVVVDVSHEDPEVKFNWYVDGVEVHNAKTK |
| | | PREEQYNSTYRVVSVLTVLHQDWLNGKEYKCKVSNKALPAPI |
| | | EKTISKAKGQPREPQVYTLPPCREEMTKNQVSLWCLVKGFYPS |
| | | DIAVEWESNGQPENNYKTTPPVLDSDGSFFLYSKLTVDKSRWQ |
| | | QGNVFSCSVMEEALENHYTQKSLSLSPGXGGGGSGGGGSGGG |
| | | GSIPPHVQKSVNNDMIVTDNNGAVKFPQLCKFCDVRFSTCDNQ |
| | | KSCMSNCSITSICEKPQEVCVAVWRKNDENITLETVCHDPKLPY |
| | | HDFILEDAASPKCIMKEKKKPGETFFMCSCSSDECNDNIIFSEEY |
| | | NTSNPD, wherein X is K or absent |
| SEQ ID NO: | hFc Hole-3x4GS- | DKTHTCPPCPAPELLGGPSVFLFPPKPKDTLMISRTPEVTCVVV |
| 194 | TGFbR2 | DVSHEDPEVKFNWYVDGVEVHNAKTKPREEQYNSTYRVVSV |
| | | LTVLHQDWLNGKEYKCKVSNKALPAPIEKTISKAKGQPREPQV |
| | | CTLPPSREEMTKNQVSLSCAVKGFYPSDIAVEWESNGQPENNY |
| | | KTTPPVLDSDGSFFLVSKLTVDKSRWQQGNVFSCSVMHEALH |
| | | NHYTQKSLSLSPGXGGGGSGGGGSGGGGSIPPHVQKSVNNDMI |
| | | VTDNNGAVKFPQLCKFCDVRFSTCDNQKSCMSNCSITSICEKP |
| | | QEVCVAVWRKNDENITLETVCHDPKLPYHDFILEDAASPKCIM |
| | | KEKKKPGETFFMCSCSSDECNDNIIFSEEYNTSNPD, wherein |
| | | X is K or absent |
| SEQ ID NO: | hFc Knob-3x4GS- | DKTHTCPPCPAPELLGGPSVFLFPPKPKDTLMISRTPEVTCVVV |
| 195 | TGFbR2 | DVSHEDPEVKFNWYVDGVEVHNAKTKPREEQYNSTYRVVSV |
| | | LTVLHQDWLNGKEYKCKVSNKALPAPIEKTISKAKGQPREPQV |
| | | YTLPPCREEMTKNQVSLWCLVKGFYPSDIAVEWESNGQPENN |
| | | YKTTPPVLDSDGSFFLYSKLTVDKSRWQQGNVFSCSVMHEAL |
| | | HNHYTQKSLSLSPGXGGGGSGGGGSGGGGSIPPHVQKSVNND |
| | | MIVTDNNGAVKFPQLCKFCDVRFSTCDNQKSCMSNCSITSICEK |
| | | PQEVCVAVWRKNDENITLETVCHDPKLPYHDFILEDAASPKCI |
| | | MKEKKKPGETFFMCSCSSDECNDNIIFSEEYNTSNPD, wherein X |
| | | is K or absent |
| SEQ ID NO: | TGFbR2-3x4GS- | IPPHVQKSVNNDMIVTDNNGAVKFPQLCKFCDVRFSTCDNQKS |
| 196 | hCH1-hFc_Hole | CMSNCSITSICEKPQEVCVAVWRKNDENITLETVCHDPKLPYH |
| | | DFILEDAASPKCIMKEKKKPGETFFMCSCSSDECNDNIIFSEEYN |
| | | TSNPDGGGGSGGGGSGGGGSASTKGPSVFPLAPSSKSTSGGTA |
| | | ALGCLVKDYFPEPVTVSWNSGALTSGVHTFPAVLQSSGLYSLS |
| | | SVVTVPSSSLGTQTYICNVNHKPSNTKVDKRVEPKSCDKTHTC |
| | | PPCPAPELLGGPSVFLFPPKPKDTLMISRTPEVTCVVVDVSHED |
| | | PEVKFNWYVDGVEVHNAKTKPREEQYNSTYRVVSVLTVLHQ |
| | | DWLNGKEYKCKVSNKALPAPIEKTISKAKGQPREPQVCTLPPS |
| | | REEMTKNQVSLSCAVKGFYPSDIAVEWESNGQPENNYKTTPPV |
| | | LDSDGSFFLVSKLTVDKSRWQQGNVFSCSVMHEALHNHYTQK |
| | | SLSLSPGX, wherein X is K or absent |
| SEQ ID NO: | TGFbR2-3x4G5- | IPPHVQKSVNNDMIVTDNNGAVKFPQLCKFCDVRFSTCDNQKS |
| 197 | hCH1-hFc_Knob | CMSNCSITSICEKPQEVCVAVWRKNDENITLETVCHDPKLPYH |
| | | DFILEDAASPKCIMKEKKKPGETFFMCSCSSDECNDNIIFSEEYN |
| | | TSNPDGGGGSGGGGSGGGGSASTKGPSVFPLAPSSKSTSGGTA |
| | | ALGCLVKDYFPEPVTVSWNSGALTSGVHTFPAVLQSSGLYSLS |
| | | SVVTVPSSSLGTQTYICNVNHKPSNTKVDKRVEPKSCDKTHTC |
| | | PPCPAPELLGGPSVFLFPPKPKDTLMISRTPEVTCVVVDVSHED |
| | | PEVKFNWYVDGVEVHNAKTKPREEQYNSTYRVVSVLTVLHQ |
| | | DWLNGKEYKCKVSNKALPAPIEKTISKAKGQPREPQVYTLPPC |
| | | REEMTKNQVSLWCLVKGFYPSDIAVEWESNGQPENNYKTTPP |
| | | VLDSDGSFFLYSKLTVDKSRWQQGNVFSCSVMHEALHNHYTQ |
| | | KSLSLSPGX, wherein X is K or absent |
| SEQ ID NO: | TGFbR2-3x4G5- | IPPHVQKSVNNDMIVTDNNGAVKFPQLCKFCDVRFSTCDNQKS |
| 198 | hCLIg_v1 | CMSNCSITSICEKPQEVCVAVWRKNDENITLETVCHDPKLPYH |
| | | DFILEDAASPKCIMKEKKKPGETFFMCSCSSDECNDNIIFSEEYN |
| | | TSNPDGGGGSGGGGSGGGGSGQPKANPTVTLFPPSSEELQANK |
| | | ATLVCLISDFYPGAVTVAWKADGSPVKAGVETTKPSKQSNNK |
| | | YAASSYLSLTPEQWKSHRSYSCQVTHEGSTVEKTVAPTECS |
| SEQ ID NO: | TGFOR2-3x4G5- | IPPHVQKSVNNDMIVTDNNGAVKFPQLCKFCDVRFSTCDNQKS |
| 199 | hCLIg_vk | CMSNCSITSICEKPQEVCVAVWRKNDENITLETVCHDPKLPYH |
| | | DFILEDAASPKCIMKEKKKPGETFFMCSCSSDECNDNIIFSEEYN |
| | | TSNPDGGGGSGGGGSGGGGSRTVAAPSVFIFPPSDEQLKSGTAS |
| | | VVCLLNNFYPREAKVQWKVDNALQSGNSQESVTEQDSKDSTY |
| | | SLSSTLTLSKADYEKHKVYACEVTHQGLSSPVTKSFNRGEC |

### Cytokine Molecules

Cytokines are generally polypeptides that influence cellular activity, for example, through signal transduction pathways. Accordingly, a cytokine of the multispecific or multifunctional polypeptide is useful and can be associated with receptor-mediated signaling that transmits a signal from outside the cell membrane to modulate a response within the cell. Cytokines are proteinaceous signaling compounds that are mediators of the immune response. They control many different cellular functions including proliferation, differentiation and cell survival/apoptosis; cytokines are also involved in several pathophysiological processes including viral infections and autoimmune diseases. Cytokines are synthesized under various stimuli by a variety of cells of both the innate (monocytes, macrophages, dendritic cells) and adaptive (T- and B-cells) immune systems. Cytokines can be classified into two groups: pro- and anti-inflammatory. Pro-inflammatory cytokines, including IFNγ, IL-1, IL-6 and TNF-alpha, are predominantly derived from the innate immune cells and Th1 cells. Anti-inflammatory cytokines, including IL-10, IL-4, IL-13 and IL-5, are synthesized from Th2 immune cells.

The present disclosure provides, inter alia, multispecific (e.g., bi-, tri-, quad-specific) or multifunctional molecules, that include, e.g., are engineered to contain, one or more cytokine molecules, e.g., immunomodulatory (e.g., proinflammatory) cytokines and variants, e.g., functional variants, thereof. Accordingly, in some embodiments, the cytokine molecule is an interleukin or a variant, e.g., a functional variant thereof. In some embodiments the interleukin is a proinflammatory interleukin. In some embodiments the interleukin is chosen from interleukin-2 (IL-2), interleukin-12 (IL-12), interleukin-15 (IL-15), interleukin-18 (IL-18), interleukin-21 (IL-21), interleukin-7 (IL-7), or interferon gamma. In some embodiments, the cytokine molecule is a proinflammatory cytokine.

In certain embodiments, the cytokine is a single chain cytokine. In certain embodiments, the cytokine is a multichain cytokine (e.g., the cytokine comprises 2 or more (e.g., 2) polypeptide chains. An exemplary multichain cytokine is IL-12.

Examples of useful cytokines include, but are not limited to, GM-CSF, IL-1α, IL-1β, IL-2, IL-3, IL-4, IL-5, IL-6, IL-7, IL-8, IL-10, IL-12, IL-21, IFN-α, IFN-β, IFN-γ, MIP-1α, MIP-1β, TGF-β, TNF-α, and TNFβ. In one embodiment the cytokine of the multispecific or multifunctional polypeptide is a cytokine selected from the group of GM-CSF, IL-2, IL-7, IL-8, IL-10, IL-12, IL-15, IL-21, IFN-α, IFN-γ, MIP-1α, MIP-1β and TGF-β. In one embodiment the cytokine of the i the multispecific or multifunctional polypeptide is a cytokine selected from the group of IL-2, IL-7, IL-10, IL-12, IL-15, IFN-α, and IFN-γ. In certain embodiments the cytokine is mutated to remove N- and/or O-glycosylation sites. Elimination of glycosylation increases homogeneity of the product obtainable in recombinant production.

In one embodiment, the cytokine of the multispecific or multifunctional polypeptide is IL-2. In a specific embodiment, the IL-2 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity. In another particular embodiment the IL-2 cytokine is a mutant IL-2 cytokine having reduced binding affinity to the .alpha.-subunit of the IL-2 receptor. Together with the .beta.- and .gamma.-subunits (also known as CD122 and CD132, respectively), the .alpha.-subunit (also known as CD25) forms the heterotrimeric high-affinity IL-2 receptor, while the dimeric receptor consisting only of the β- and γ-subunits is termed the intermediate-affinity IL-2 receptor. As described in PCT patent application number PCT/EP2012/051991, which is incorporated herein by reference in its entirety, a mutant IL-2 polypeptide with reduced binding to the .alpha.-subunit of the IL-2 receptor has a reduced ability to induce IL-2 signaling in regulatory T cells, induces less activation-induced cell death (AICD) in T cells, and has a reduced toxicity profile in vivo, compared to a wild-type IL-2 polypeptide. The use of such a cytokine with reduced toxicity is particularly advantageous in a multispecific or multifunctional polypeptide according to the invention, having a long serum half-life due to the presence of an Fc domain. In one embodiment, the mutant IL-2 cytokine of the multispecific or multifunctional polypeptide according to the invention comprises at least one amino acid mutation that reduces or abolishes the affinity of the mutant IL-2 cytokine to the .alpha.-subunit of the IL-2 receptor (CD25) but preserves the affinity of the mutant IL-2 cytokine to the intermediate-affinity IL-2 receptor (consisting of the β and γ subunits of the IL-2 receptor), compared to the non-mutated IL-2 cytokine. In one embodiment the one or more amino acid mutations are amino acid substitutions. In a specific embodiment, the mutant IL-2 cytokine comprises one, two or three amino acid substitutions at one, two or three position(s) selected from the positions corresponding to residue 42, 45, and 72 of human IL-2. In a more specific embodiment, the mutant IL-2 cytokine comprises three amino acid substitutions at the positions corresponding to residue 42, 45 and 72 of human IL-2. In an even more specific embodiment, the mutant IL-2 cytokine is human IL-2 comprising the amino acid substitutions F42A, Y45A and L72G. In one embodiment the mutant IL-2 cytokine additionally comprises an amino acid mutation at a position corresponding to position 3 of human IL-2, which eliminates the O-glycosylation site of IL-2. Particularly, said additional amino acid mutation is an amino acid substitution replacing a threonine residue by an alanine residue. A particular mutant IL-2 cytokine useful in the invention comprises four amino acid substitutions at positions corresponding to residues 3, 42, 45 and 72 of human IL-2. Specific amino acid substitutions are T3A, F42A, Y45A and L72G. As demonstrated in PCT patent application number PCT/EP2012/051991 and in the appended Examples, said quadruple mutant IL-2 polypeptide (IL-2 qm) exhibits no detectable binding to CD25, reduced ability to induce apoptosis in T cells, reduced ability to induce IL-2 signaling in T.sub.reg cells, and a reduced toxicity profile in vivo. However, it retains ability to activate IL-2 signaling in effector cells, to induce proliferation of effector cells, and to generate IFN-γ as a secondary cytokine by NK cells.

The IL-2 or mutant IL-2 cytokine according to any of the above embodiments may comprise additional mutations that provide further advantages such as increased expression or stability. For example, the cysteine at position 125 may be replaced with a neutral amino acid such as alanine, to avoid the formation of disulfide-bridged IL-2 dimers. Thus, in certain embodiments the IL-2 or mutant IL-2 cytokine of the multispecific or multifunctional polypeptide according to the invention comprises an additional amino acid mutation at a position corresponding to residue 125 of human IL-2. In one embodiment said additional amino acid mutation is the amino acid substitution C125A.

In a specific embodiment the IL-2 cytokine of the multispecific or multifunctional polypeptide comprises the polypeptide sequence of SEQ ID NO: 237 [APTSSSTKKTQLQLEHLLLDLQMILNGINNYKNPKLTRMLTFKFYMPKKATELKHLQCL EEELKPLEEVLNLAQSKNFHLRPRDLISNINVIVLELKGSETTFMCEYADETATIVEFLNR WITFAQSIISTLT]. In another specific embodiment the IL-2 cytokine of the multispecific or multifunctional polypeptide comprises the polypeptide sequence of SEQ ID NO: 238

[APASSSTKKTQLQLEHLLLDLQMILNGINNYKNPKLTRMLTAKFAMPK
KATELKHLQCLEEELKPLEEVLNGAQSKNFHLRPRDLISNINVIVLELK
GSETTFMCEYADETATIVEFLNRWITFAQSIISTLT].

In another embodiment the cytokine of the multispecific or multifunctional polypeptide is IL-12. In a specific embodiment said IL-12 cytokine is a single chain IL-12 cytokine. In an even more specific embodiment the single chain IL-12 cytokine comprises the polypeptide sequence of SEQ ID NO: 239 [IWELKKDVYVVELDWYPDAPGEMVVLTCDTPEEDGITWTLDQSSEVLGSGKTLTIQVK EFGDAGQYTCHKGGEVLSHSLLLLHKKEDGIWSTDILKDQKEPKNKTFLRCEAKNYSGR FTCWWLTTISTDLTFSVKSSRGSSDPQGVTCGAATLSAERVRGDNKEYEYSVECQEDSA CPAAEESLPIEVMVDAVHKLKYENYTSSFFIRDIIKPDPPKNLQLKPLKNSRQVEVSWEY PDTWSTPHSYFSLTFCVQVQGKSKREKKDRVFTDKTSATVICRKNASISVRAQDRYYSS SWSEWASVPCSGGGGSGGGGSGGGGSRNLPVATPDPGMFPCLHHSQNLLRAVSNMLQ KARQTLEFYPCTSEEIDHEDITKDKTSTVEACLPLELTKNESCLNSRETSFITNGSCLASRK TSFMMALCLSSIYEDLKMYQVEFKTMNAKLLMDPKRQIFLDQNMLAVIDELMQALNFN SETVPQKSSLEEPDFYKTKIKLCILLHAFRIRAVTIDRVMSYLNAS]. In one embodiment, the IL-12 cytokine can elicit one or more of the cellular responses selected from the group consisting of: proliferation in a NK cell, differentiation in a NK cell, proliferation in a T cell, and differentiation in a T cell.

In another embodiment the cytokine of the multispecific or multifunctional polypeptide is IL-10. In a specific embodiment said IL-10 cytokine is a single chain IL-10 cytokine. In an even more specific embodiment the single chain IL-10 cytokine comprises the polypeptide sequence of SEQ ID NO: 240 [SPGQGTQSENSCTHFPGNLPNMLRDLRDAFSRVKTFFQMKDQLDNLLLKESLLEDFKG YLGCQALSEMIQFYLEEVMPQAENQDPDIKAHVNSLGENLKTLRLRLRRCHRFLPCENK SKAVEQVKNAFNKLQEKGIYKAMSEFDIFINYIEAYMTMKIRNGGGGSGGGGSGGGGS GGGGSSPGQGTQSENSCTHFPGNLPNMLRDLRDAFSRVKTFFQMKDQLDNLLLKESLLE DFKGYLGCQALSEMIQFYLEEVMPQAENQDPDIKAHVNSLGENLKTLRLRLRRCHRFLP CENKSKAVEQVKNAFNKLQEKGIYKAMSEFDIFINYIEAYMTMKIRN]. In another specific embodiment the IL-10 cytokine is a monomeric IL-10 cytokine. In a more specific embodiment the monomeric IL-10 cytokine comprises the polypeptide sequence of SEQ ID NO: 241 [SPGQGTQSENSCTHFPGNLPNMLRDLRDAFSRVKTFFQMKDQLDNLLLKESLLEDFKG YLGCQALSEMIQFYLEEVMPQAENQDPDIKAHVNSLGENLKTLRLRLRRCHRFLPCENG GGSGGKSKAVEQVKNAFNKLQEKGIYKAMSEFDIFINYIEAYMTMKIRN]. In one embodiment, the IL-10 cytokine can elicit one or more of the cellular responses selected from the group consisting of: inhibition of cytokine secretion, inhibition of antigen presentation by antigen presenting cells, reduction of oxygen radical release, and inhibition of T cell proliferation. A multispecific or multifunctional polypeptide according to the invention wherein the cytokine is IL-10 is particularly useful for downregulation of inflammation, e.g. in the treatment of an inflammatory disorder.

In another embodiment, the cytokine of the multispecific or multifunctional polypeptide is IL-15. In a specific embodiment said IL-15 cytokine is a mutant IL-15 cytokine having reduced binding affinity to the α-subunit of the IL-15 receptor. Without wishing to be bound by theory, a mutant IL-15 polypeptide with reduced binding to the .alpha.-subunit of the IL-15 receptor has a reduced ability to bind to fibroblasts throughout the body, resulting in improved pharmacokinetics and toxicity profile, compared to a wild-type IL-15 polypeptide. The use of an cytokine with reduced toxicity, such as the described mutant IL-2 and mutant IL-15 effector moieties, is particularly advantageous in a multispecific or multifunctional polypeptide according to the invention, having a long serum half-life due to the presence of an Fc domain. In one embodiment the mutant IL-15 cytokine of the multispecific or multifunctional polypeptide according to the invention comprises at least one amino acid mutation that reduces or abolishes the affinity of the mutant IL-15 cytokine to the .alpha.-subunit of the IL-15 receptor but preserves the affinity of the mutant IL-15 cytokine to the intermediate-affinity IL-15/IL-2 receptor (consisting of the .beta.- and .gamma.-subunits of the IL-15/IL-2 receptor), compared to the non-mutated IL-15 cytokine. In one embodiment the amino acid mutation is an amino acid substitution. In a specific embodiment, the mutant IL-15 cytokine comprises an amino acid substitution at the position corresponding to residue 53 of human IL-15. In a more specific embodiment, the mutant IL-15 cytokine is human IL-15 comprising the amino acid substitution E53A. In one embodiment the mutant IL-15 cytokine additionally comprises an amino acid mutation at a position corresponding to position 79 of human IL-15, which eliminates the N-glycosylation site of IL-15. Particularly, said additional amino acid mutation is an amino acid substitution replacing an asparagine residue by an alanine residue. In an even more specific embodiment the IL-15 cytokine comprises the polypeptide sequence of SEQ ID NO: 242 [NWVNVISDLKKIEDLIQSMHIDATLYTESDVHPSCKVTAMKCFLLELQVISLASGDASIH DTVENLIILANNSLSSNGAVTESGCKECEELEEKNIKEFLQSFVHIVQMFINTS]. In one embodiment, the IL-15 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

Mutant cytokine molecules useful as effector moieties in the multispecific or multifunctional polypeptide can be prepared by deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing. Substitution or insertion may involve natural as well as non-natural amino acid residues. Amino acid modification includes well known methods of chemical modification such as the addition or removal of glycosylation sites or carbohydrate attachments, and the like.

In one embodiment, the cytokine, particularly a single-chain cytokine, of the multispecific or multifunctional polypeptide is GM-CSF. In a specific embodiment, the GM-CSF cytokine can elicit proliferation and/or differentiation in a granulocyte, a monocyte or a dendritic cell. In one embodiment, the cytokine, particularly a single-chain cytokine, of the multispecific or multifunctional polypeptide is IFN-α. In a specific embodiment, the IFN-α cytokine can elicit one or more of the cellular responses selected from the group consisting of: inhibiting viral replication in a virus-infected cell, and upregulating the expression of major histocompatibility complex I (MHC I). In another specific embodiment, the IFN-α cytokine can inhibit proliferation in a tumor cell. In one embodiment the cytokine, particularly a single-chain cytokine, of the multispecific or multifunctional polypeptide is IFNγ. In a specific embodiment, the IFN-γ cytokine can elicit one or more of the cellular responses selected from the group of: increased macrophage activity, increased expression of MHC molecules, and increased NK cell activity. In one embodiment the cytokine, particularly a single-chain cytokine, of the multispecific or multifunctional polypeptide is IL-7. In a specific embodiment, the IL-7 cytokine can elicit proliferation of T and/or B lymphocytes. In one embodiment, the cytokine, particularly a single-chain cytokine, of the multispecific or multifunctional polypeptide is IL-8. In a specific embodiment, the IL-8 cytokine can elicit chemotaxis in neutrophils. In one embodiment, the cytokine, particularly a single-chain cytokine, of the multispecific or multifunctional polypeptide, is MIP-1α. In a specific embodiment, the MIP-1αcytokine can elicit chemotaxis in monocytes and T lymphocyte cells. In one embodiment, the cytokine, particularly a single-chain cytokine, of the multispecific or multifunctional polypeptide is MIP-1β. In a specific embodiment, the MIP-1β cytokine can elicit chemotaxis in monocytes and T lymphocyte cells. In one embodiment, the cytokine, particularly a single-chain cytokine, of the multispecific or multifunctional polypeptide is TGF-β. In a specific embodiment, the TGF-β cytokine can elicit one or more of the cellular responses selected from the group consisting of: chemotaxis in monocytes, chemotaxis in macrophages, upregulation of IL-1 expression in activated macrophages, and upregulation of IgA expression in activated B cells.

In one embodiment, the multispecific or multifunctional polypeptide of the invention binds to an cytokine receptor with a dissociation constant (K_{D}) that is at least about 1, 1.5, 2, 2.5, 3, 3.5, 4, 4.5, 5, 5.5, 6, 6.5, 7, 7.5, 8, 8.5, 9, 9.5 or 10 times greater than that for a control cytokine. In another embodiment, the multispecific or multifunctional polypeptide binds to an cytokine receptor with a K_{D} that is at least 2, 3, 4, 5, 6, 7, 8, 9, or 10 times greater than that for a corresponding multispecific or multifunctional polypeptide comprising two or more effector moieties. In another embodiment, the multispecific or multifunctional polypeptide binds to an cytokine receptor with a dissociation constant K_{D} that is about 10 times greater than that for a corresponding the multispecific or multifunctional polypeptide comprising two or more cytokines.

In some embodiments, the multispecific molecules disclosed herein include a cytokine molecule. In embodiments, the cytokine molecule includes a full length, a fragment or a variant of a cytokine; a cytokine receptor domain, e.g., a cytokine receptor dimerizing domain; or an agonist of a cytokine receptor, e.g., an antibody molecule (e.g., an agonistic antibody) to a cytokine receptor.

In some embodiments the cytokine molecule is chosen from IL-2, IL-12, IL-15, IL-18, IL-7, IL-21, or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain.

In other embodiments, the cytokine molecule is an agonist of a cytokine receptor, e.g., an antibody molecule (e.g., an agonistic antibody) to a cytokine receptor chosen from an IL-15Ra or IL-21R.

In one embodiment, the cytokine molecule is IL-15, e.g., human IL-15 (e.g., comprising the amino acid sequence: NWVNVISDLKKIEDLIQSMHIDATLYTESDVHPSCKVTAMKCFLLELQVISLESGDASIH DTVENLIILANNSLSSNGNVTESGCKECEELEEKNIKEFLQSFVHIVQMFINTS (SEQ ID NO: 243), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 243.

In some embodiments, the cytokine molecule comprises a receptor dimerizing domain, e.g., an IL15Ralpha dimerizing domain. In one embodiment, the IL15Ralpha dimerizing domain comprises the amino acid sequence: MAPRRARGCRTLGLPALLLLLLLRPPATRGITCPPPMSVEHADIWVKSYSLYSRERYICN SGFKRKAGTSSLTECVL (SEQ ID NO: 244), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 244. In some embodiments, the cytokine molecule (e.g., IL-15) and the receptor dimerizing domain (e.g., an IL15Ralpha dimerizing domain) of the multispecific molecule are covalently linked, e.g., via a linker (e.g., a Gly-Ser linker, e.g., a linker comprising the amino acid sequence SGGSGGGGSGGGSGGGGSLQ (SEQ ID NO: 245). In other embodiments, the cytokine molecule (e.g., IL-15) and the receptor dimerizing domain (e.g., an IL15Ralpha dimerizing domain) of the multispecific molecule are not covalently linked, e.g., are non-covalently associated.

In other embodiments, the cytokine molecule is IL-2, e.g., human IL-2 (e.g., comprising the amino acid sequence: APTSSSTKKTQLQLEHLLLDLQMILNGINNYKNPKLTRMLTFKFYMPKKATELKHLQCL EEELKPLEEVLNLAQSKNFHLRPRDLISNINVIVLELKGSETTFMCEYADETATIVEFLNR WITFCQSIISTLT (SEQ ID NO: 246), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 246).

In other embodiments, the cytokine molecule is IL-18, e.g., human IL-18 (e.g., comprising the amino acid sequence: YFGKLESKLSVIRNLNDQVLFIDQGNRPLFEDMTDSDCRDNAPRTIFIISMYKDSQPRGM AVTISVKCEKISTLSCENKIISFKEMNPPDNIKDTKSDIIFFQRSVPGHDNKMQFESSSY EGYFLACEKERDLFKLILKKEDELGDRSIMFTVQNED (SEQ ID NO: 247), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 247).

In other embodiments, the cytokine molecule is IL-21, e.g., human IL-21 (e.g., comprising the amino acid sequence: QGQDRHMIRMRQLIDIVDQLKNYVNDLVPEFLPAPEDVETNCEWSAFSCFQKAQLKSA NTGNNERIINVSIKKLKRKPPSTNAGRRQKHRLTCPSCDSYEKKPPKEFLERFKSLLQKMI HQHLSSRTHGSEDS (SEQ ID NO: 248), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 248).

In yet other embodiments, the cytokine molecule is interferon gamma, e.g., human interferon gamma (e.g., comprising the amino acid sequence: QDPYVKEAENLKKYFNAGHSDVADNGTLFLGILKNWKEESDRKIMQSQIVSFYFKLFK NFKDDQSIQKSVETIKEDMNVKFFNSNKKKRDDFEKLTNYSVTDLNVQRKAIHELIQVM AELSPAAKTGKRKRSQMLFRG (SEQ ID NO: 249), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 249).

### Nucleic Acids

The invention also features nucleic acids comprising nucleotide sequences that encode heavy and light chain variable regions and CDRs or hypervariable loops of the antibody molecules, as described herein. For example, the invention features a first and second nucleic acid encoding heavy and light chain variable regions, respectively, of an antibody molecule chosen from one or more of the antibody molecules disclosed herein. The nucleic acid can comprise a nucleotide sequence as set forth in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

In certain embodiments, the nucleic acid can comprise a nucleotide sequence encoding at least one, two, or three CDRs or hypervariable loops from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs or hypervariable loops from a heavy chain variable region having the nucleotide sequence as set forth in the tables herein, a sequence substantially homologous thereto (e.g., a sequence at least about 85%, 90%, 95%, 99% or more identical thereto, and/or capable of hybridizing under the stringency conditions described herein). In another embodiment, the nucleic acid can comprise a nucleotide sequence encoding at least one, two, or three CDRs or hypervariable loops from a light chain variable region having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein).

In another aspect, the application features host cells and vectors containing the nucleic acids described herein. The nucleic acids may be present in a single vector or separate vectors present in the same host cell or separate host cell, as described in more detail herein below.

### Vectors

Further provided herein are vectors comprising the nucleotide sequences encoding an antibody molecule described herein. In one embodiment, the vectors comprise nucleotides encoding an antibody molecule described herein. In one embodiment, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

In another aspect, the application features host cells and vectors containing the nucleic acids described herein. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell. The invention also provides host cells comprising a nucleic acid encoding an antibody molecule as described herein.

In one embodiment, the host cells are genetically engineered to comprise nucleic acids encoding the antibody molecule.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The invention also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### Uses and Combination Therapies

The multispecific molecule described herein, alone or in combination with a second therapy or a second therapeutic agent, can be used to treat a hyperproliferative disorder, a cancer, or a fibrotic disorder.

### Cancer

Methods described herein include treating a cancer in a subject by using a multispecific molecule described herein, e.g., using a pharmaceutical composition described herein. Also provided are methods for reducing or ameliorating a symptom of a cancer in a subject, as well as methods for inhibiting the growth of a cancer and/or killing one or more cancer cells. In embodiments, the methods described herein decrease the size of a tumor and/or decrease the number of cancer cells in a subject administered with a described herein or a pharmaceutical composition described herein.

In embodiments, the cancer is a hematological cancer. In embodiments, the hematological cancer is a leukemia or a lymphoma. As used herein, a “hematologic cancer” refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sezary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

In embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi's sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In certain embodiments, the cancer is an epithelial, mesenchymal or hematologic malignancy. In certain embodiments, the cancer treated is a solid tumor (e.g., carcinoid, carcinoma or sarcoma), a soft tissue tumor (e.g., a heme malignancy), and a metastatic lesion, e.g., a metastatic lesion of any of the cancers disclosed herein. In one embodiment, the cancer treated is a fibrotic or desmoplastic solid tumor, e.g., a tumor having one or more of: limited tumor perfusion, compressed blood vessels, fibrotic tumor interstitium, or increased interstitial fluid pressure. In one embodiment, the solid tumor is chosen from one or more of pancreatic (e.g., pancreatic adenocarcinoma or pancreatic ductal adenocarcinoma), breast, colon, colorectal, lung (e.g., small cell lung cancer (SCLC) or non-small cell lung cancer (NSCLC)), skin, ovarian, liver cancer, esophageal cancer, endometrial cancer, gastric cancer, head and neck cancer, kidney, or prostate cancer.

Examples of cancer include, but are not limited to, carcinoma, lymphoma, blastoma, sarcoma, and leukemia or lymphoid malignancies. More particular examples of such cancers are noted below and include: squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenström's Macroglobulinemia, Wilms' Tumor, and any other hyperproliferative disease, besides neoplasia, located in an organ system listed above.

In other embodiments, the multispecific molecule, as described above and herein, is used to treat a hyperproliferative disorder, e.g., a hyperproliferative connective tissue disorder (e.g., a hyperproliferative fibrotic disease). In one embodiment, the hyperproliferative fibrotic disease is multisystemic or organ-specific. Exemplary hyperproliferative fibrotic diseases include, but are not limited to, multisystemic (e.g., systemic sclerosis, multifocal fibrosclerosis, sclerodermatous graft-versus-host disease in bone marrow transplant recipients, nephrogenic systemic fibrosis, scleroderma), and organ-specific disorders (e.g., fibrosis of the eye, lung, liver, heart, kidney, pancreas, skin and other organs). In other embodiments, the disorder is chosen from liver cirrhosis or tuberculosis. In other embodiments, the disorder is leprosy.

In embodiments, the multispecific molecules (or pharmaceutical composition) are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition (or multispecific molecules) to be administered can be determined by a physician with consideration of individual differences in tumor size, extent of infection or metastasis, age, weight, and condition of the subject. In embodiments, the pharmaceutical composition described herein can be administered at a dosage of 10⁴ to 10⁹ cells/kg body weight, e.g., 10⁵ to 10⁶ cells/kg body weight, including all integer values within those ranges. In embodiments, the pharmaceutical composition described herein can be administered multiple times at these dosages. In embodiments, the pharmaceutical composition described herein can be administered using infusion techniques described in immunotherapy (see, e.g., Rosenberg et al., New Eng. J. of Med. 319:1676, 1988).

In embodiments, the multispecific molecules or pharmaceutical composition is administered to the subject parenterally. In embodiments, the cells are administered to the subject intravenously, subcutaneously, intratumorally, intranodally, intramuscularly, intradermally, or intraperitoneally. In embodiments, the cells are administered, e.g., injected, directly into a tumor or lymph node. In embodiments, the cells are administered as an infusion (e.g., as described in Rosenberg et al., New Eng. J. of Med. 319:1676, 1988) or an intravenous push. In embodiments, the cells are administered as an injectable depot formulation.

In embodiments, the subject is a mammal. In embodiments, the subject is a human, monkey, pig, dog, cat, cow, sheep, goat, rabbit, rat, or mouse. In embodiments, the subject is a human. In embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### Liver Conditions or Disorders

This invention also provides methods of treating liver conditions or disorders using the multispecific molecules or pharmaceutical compositions described herein.

As used herein, “liver disorder therapy” refers to therapies or therapeutic agents used to treat or prevent a liver disorder described herein, and therefore encompasses liver cancer therapies and other liver disorder therapies, e.g., therapies for fibrotic liver disorders, fatty liver diseases, liver inflammation disorders, autoimmune liver diseases, and liver disorders induced by genetic diseases, alcoholism, drug toxicity, infection, or injury.

Examples of liver cancers include: hepatocellular carcinoma (HCC), primary liver cell carcinoma, hepatoma, fibrolamellar carcinoma, focal nodular hyperplasia, cholangiosarcoma, intrahepatic bile duct cancer, angiosarcoma or hemangiosarcoma, hepatic adenoma, hepatic hemangiomas, hepatic hamartoma, hepatoblastoma, infantile hemangioendothelialoma, mixed tumors of the liver, tumors of mesenchymal tissue, sarcoma of the liver. Examples of cancers that may metastasize to the liver include: breast cancer, colorectal cancer, esophageal cancer, kidney or renal cancer, lung cancer, ovarian cancer, pancreatic cancer, rectal cancer, skin cancer (e.g., melanoma), gastric or stomach cancer (including gastrointestinal cancer), and uterine cancer.

In an embodiment, the liver disorder is a fibrotic disorder or connective tissue disorder affecting the function or physiology of the liver. In one embodiment, the fibrotic disorder or connective tissue disorder can be systemic (affecting the whole body), multi-organ, or organ-specific (e.g., liver-specific). Examples of fibrotic liver disorders include liver fibrosis (hepatic fibrosis), liver cirrhosis, and any disorder associated with accumulation of extracellular matrix proteins, e.g., collagen, in the liver, liver scarring, and/or abnormal hepatic vasculature. Liver fibrosis is caused by liver inflammation or damage which triggers the accumulation of extracellular matrix proteins, including collagens, and scar tissue in the liver. Liver cirrhosis is the end stage of liver fibrosis, involves regenerative nodules (as a result of repair processes), and is accompanied with the distortion of the hepatic vasculature. Liver fibrotic disorders are most commonly caused by chronic viral infection (e.g., hepatitis B, hepatitis C), alcoholism, and fatty liver disease.

Examples of fatty liver diseases include fatty liver (or FLD), alcoholic liver disease, non-alcoholic fatty liver disease (NAFLD), non-alcoholic steatohepatitis (NASH), alcoholic steatohepatitis, simple steatosis, Reye's syndrome, and any disorder associated with abnormal retention of lipids in liver cells.

In one embodiment, the liver disease is NASH.

Metabolic disorders can also affect the liver and cause liver damage. Examples of metabolic disorders of the liver or affecting the liver include hemochromatosis, diabetes, obesity, hypertension, dyslipidemia, galactosemia, and glycogen storage disease.

Autoimmune disorders of the liver or affecting the liver can include systemic disorders or disorders that primarily affect an organ other than the liver, but with secondary effects to liver cells or liver function. Examples of such autoimmune disorders include autoimmune hepatitis (AIH), autoimmune liver disease, lupoid hepatitis, systemic lupus erythematosus, primary biliary cirrhosis (PBC), scleroderma, and systemic scerlosis.

### Fibrotic Conditions or Disorders

In another aspect, the invention features a method of treating or preventing a fibrotic condition or disorder in a subject. The method includes administering the multispecific molecule, as a single agent or in combination with another agent or therapeutic modality, to a subject in need thereof, in an amount sufficient to decrease or inhibit the fibrotic condition in the subject.

In certain embodiments, reducing fibrosis, or treatment of a fibrotic condition, includes reducing or inhibiting one or more of: formation or deposition of tissue fibrosis; reducing the size, cellularity (e.g., fibroblast or immune cell numbers), composition; or cellular content, of a fibrotic lesion; reducing the collagen or hydroxyproline content, of a fibrotic lesion; reducing expression or activity of a fibrogenic protein; reducing fibrosis associated with an inflammatory response; decreasing weight loss associated with fibrosis; or increasing survival.

In certain embodiments, the fibrotic condition is primary fibrosis. In one embodiment, the fibrotic condition is idiopathic. In other embodiments, the fibrotic condition is associated with (e.g., is secondary to) a disease (e.g., an infectious disease, an inflammatory disease, an autoimmune disease, a malignant or cancerous disease, and/or a connective disease); a toxin; an insult (e.g., an environmental hazard (e.g., asbestos, coal dust, polycyclic aromatic hydrocarbons), cigarette smoking, a wound); a medical treatment (e.g., surgical incision, chemotherapy or radiation), or a combination thereof.

In certain embodiments, the fibrotic condition is a fibrotic condition of the lung, a fibrotic condition of the liver (e.g., as described herein), a fibrotic condition of the heart or vasculature, a fibrotic condition of the kidney, a fibrotic condition of the skin, a fibrotic condition of the gastrointestinal tract, a fibrotic condition of the bone marrow or a hematopoietic tissue, a fibrotic condition of the nervous system, a fibrotic condition of the eye, or a combination thereof.

In certain embodiments, the fibrotic condition is a fibrotic condition of the lung. In certain embodiments, the fibrotic condition of the lung is chosen from one or more of: pulmonary fibrosis, idiopathic pulmonary fibrosis (IPF), usual interstitial pneumonitis (UIP), interstitial lung disease, cryptogenic fibrosing alveolitis (CFA), bronchiectasis, and scleroderma lung disease. In one embodiment, the fibrosis of the lung is secondary to a disease, a toxin, an insult, a medical treatment, or a combination thereof. For example, the fibrosis of the lung can be associated with (e.g., secondary to) one or more of: a disease process such as asbestosis and silicosis; an occupational hazard; an environmental pollutant; cigarette smoking; an autoimmune connective tissue disorders (e.g., rheumatoid arthritis, scleroderma and systemic lupus erythematosus (SLE)); a connective tissue disorder such as sarcoidosis; an infectious disease, e.g., infection, particularly chronic infection; a medical treatment, including but not limited to, radiation therapy, and drug therapy, e.g., chemotherapy (e.g., treatment with as bleomycin, methotrexate, amiodarone, busulfan, and/or nitrofurantoin). In one embodiment, the fibrotic condition of the lung treated with the methods of the invention is associated with (e.g., secondary to) a cancer treatment, e.g., treatment of a cancer (e.g., squamous cell carcinoma, testicular cancer, Hodgkin's disease with bleomycin). In one embodiment, the fibrotic condition of the lung is associated with an autoimmune connective tissue disorder (e.g., scleroderma or lupus, e.g., SLE).

Pulmonary fibrosis can occur as a secondary effect in disease processes such as asbestosis and silicosis, and is known to be more prevalent in certain occupations such as coal miner, ship workers and sand blasters where exposure to environmental pollutants is an occupational hazard (Green, F H et al. (2007) Toxicol Pathol. 35:136-47). Other factors that contribute to pulmonary fibrosis include cigarette smoking, and autoimmune connective tissue disorders, like rheumatoid arthritis, scleroderma and systemic lupus erythematosus (SLE) (Leslie, K O et al. (2007) Semin Respir Crit Care Med. 28:369-78; Swigris, J J et al. (2008) Chest. 133:271-80; and Antoniou, K M et al. (2008) Curr Opin Rheumatol. 20:686-91). Other connective tissue disorders such as sarcoidosis can include pulmonary fibrosis as part of the disease (Paramothayan, S et al. (2008) Respir Med. 102:1-9), and infectious diseases of the lung can cause fibrosis as a long term consequence of infection, particularly chronic infections.

Pulmonary fibrosis can also be a side effect of certain medical treatments, particularly radiation therapy to the chest and certain medicines like bleomycin, methotrexate, amiodarone, busulfan, and nitrofurantoin (Catane, R et al. (1979) Int J Radiat Oncol Biol Phys. 5:1513-8; Zisman, D A et al. (2001) Sarcoidosis Vasc Diffuse Lung Dis. 18:243-52; Rakita, L et al. (1983) Am Heart J. 106:906-16; Twohig, K J et al. (1990) Clin Chest Med. 11:31-54; and Witten C M. (1989) Arch Phys Med Rehabil. 70:55-7). In other embodiments, idiopathic pulmonary fibrosis can occur where no clear causal agent or disease can be identified. Genetic factors can play a significant role in these cases of pulmonary fibrosis (Steele, M P et al. (2007) Respiration 74:601-8; Brass, D M et al. (2007) Proc Am Thorac Soc. 4:92-100 and du Bois R M. (2006) Semin Respir Crit Care Med. 27:581-8).

In other embodiments, pulmonary fibrosis includes, but is not limited to, pulmonary fibrosis associated with chronic obstructive pulmonary disease (COPD), acute respiratory distress syndrome, scleroderma, pleural fibrosis, chronic asthma, acute lung syndrome, amyloidosis, bronchopulmonary dysplasia, Caplan's disease, Dressler's syndrome, histiocytosis X, idiopathic pulmonary haemosiderosis, lymphangiomyomatosis, mitral valve stenosis, polymyositis, pulmonary edema, pulmonary hypertension (e.g., idiopathic pulmonary hypertension (IPH)), pneumoconiosis, radiotherapy (e.g., radiation induced fibrosis), rheumatoid disease, Shaver's disease, systemic lupus erythematosus, systemic sclerosis, tropical pulmonary eosinophilia, tuberous sclerosis, Weber-Christian disease, Wegener's granulomatosis, Whipple's disease, or exposure to toxins or irritants (e.g., pharmaceutical drugs such as amiodarone, bleomycin, busulphan, carmustine, chloramphenicol, hexamethonium, methotrexate, methysergide, mitomycin C, nitrofurantoin, penicillamine, peplomycin, and practolol; inhalation of talc or dust, e.g., coal dust, silica). In certain embodiments, the pulmonary fibrosis is associated with an inflammatory disorder of the lung, e.g., asthma, and/or COPD.

In certain embodiments, the fibrotic condition is a fibrotic condition of the liver. In certain embodiments, the fibrotic condition of the liver is chosen from one or more of: fatty liver disease, steatosis (e.g., nonalcoholic steatohepatitis (NASH), cholestatic liver disease (e.g., primary biliary cirrhosis (PBC)), cirrhosis, alcohol induced liver fibrosis, biliary duct injury, biliary fibrosis, or cholangiopathies. In other embodiments, hepatic or liver fibrosis includes, but is not limited to, hepatic fibrosis associated with alcoholism, viral infection, e.g., hepatitis (e.g., hepatitis C, B or D), autoimmune hepatitis, non-alcoholic fatty liver disease (NAFLD), progressive massive fibrosis, exposure to toxins or irritants (e.g., alcohol, pharmaceutical drugs and environmental toxins). Additional examples of liver conditions and disorders are provided in the Sections entitled “Liver Conditions or Disorders,” provided herein.

In certain embodiments, the fibrotic condition is a fibrotic condition of the kidney. In certain embodiments, the fibrotic condition of the kidney is chosen from one or more of: renal fibrosis (e.g., chronic kidney fibrosis), nephropathies associated with injury/fibrosis (e.g., chronic nephropathies associated with diabetes (e.g., diabetic nephropathy)), lupus, scleroderma of the kidney, glomerular nephritis, focal segmental glomerular sclerosis, IgA nephropathyrenal fibrosis associated with human chronic kidney disease (CKD), chronic progressive nephropathy (CPN), tubulointerstitial fibrosis, ureteral obstruction, chronic uremia, chronic interstitial nephritis, radiation nephropathy, glomerulosclerosis, progressive glomerulonephrosis (PGN), endothelial/thrombotic microangiopathy injury, HIV-associated nephropathy, or fibrosis associated with exposure to a toxin, an irritant, or a chemotherapeutic agent. In one embodiment, the fibrotic condition of the kidney is scleroderma of the kidney. In some embodiments, the fibrotic condition of the kidney is transplant nephropathy, diabetic nephropathy, lupus nephritis, focal segmental glomerulosclerosis (FSGS), endothelial/thrombotic microangiopathy injury, scleroderma of the kidney, HIV-associated nephropathy (HIVVAN), or exposure to toxins, irritants, chemotherapeutic agents.

In certain embodiments, the fibrotic condition is a fibrotic condition of the bone marrow or a hematopoietic tissue. In certain embodiments, the fibrotic condition of the bone marrow is an intrinsic feature of a chronic myeloproliferative neoplasm of the bone marrow, such as primary myelofibrosis (also referred to herein as agnogenic myeloid metaplasia or chronic idiopathic myelofibrosis). In other embodiments, the bone marrow fibrosis is associated with (e.g., is secondary to) a malignant condition or a condition caused by a clonal proliferative disease. In other embodiments, the bone marrow fibrosis is associated with a hematologic disorder (e.g., a hematologic disorder chosen from one or more of polycythemia vera, essential thrombocythemia, myelodysplasia, hairy cell leukemia, lymphoma (e.g., Hodgkin or non-Hodgkin lymphoma), multiple myeloma or chronic myelogeneous leukemia (CML)). In yet other embodiments, the bone marrow fibrosis is associated with (e.g., secondary to) a non-hematologic disorder (e.g., a non-hematologic disorder chosen from solid tumor metastasis to bone marrow, an autoimmune disorder (e.g., systemic lupus erythematosus, scleroderma, mixed connective tissue disorder, or polymyositis), an infection (e.g., tuberculosis or leprosy), or secondary hyperparathyroidism associated with vitamin D deficiency. In some embodiments, the fibrotic condition is idiopathic or drug-induced myelofibrosis. In some embodiments, the fibrotic condition of the bone marrow or hematopoietic tissue is associated with systemic lupus erythematosus or scleroderma.

In other embodiments, the fibrotic condition is associated with leprosy or tuberculosis.

In certain embodiments, the fibrotic condition is a fibrotic condition of the bone marrow. In certain embodiments, the fibrotic condition of the bone marrow is myelofibrosis (e.g., primary myelofibrosis (PMF)), myeloid metaplasia, chronic idiopathic myelofibrosis, or primary myelofibrosis. In other embodiments, bone marrow fibrosis is associated with a hematologic disorder chosen from one or more of hairy cell leukemia, lymphoma, or multiple myeloma.

In other embodiments, the bone marrow fibrosis is associated with one or more myeloproliferative neoplasms (MPN) chosen from: essential thrombocythemia (ET), polycythemia vera (PV), mastocytosis, chronic eosinophilic leukemia, chronic neutrophilic leukemia, or other MPN.

In one embodiment, the fibrotic condition is primary myelofibrosis. Primary myelofibrosis (PMF) (also referred to in the literature as idiopathic myeloid metaplasia, and Agnogenic myeloid metaplasia) is a clonal disorder of multipotent hematopoietic progenitor cells (reviewed in Abdel-Wahab, O. et al. (2009) Annu. Rev. Med. 60:233-45; Varicchio, L. et al. (2009) Expert Rev. Hematol. 2(3):315-334; Agrawal, M. et al. (2010) Cancer 1-15).

In certain embodiments, the fibrotic condition is a fibrotic condition of the heart. In certain embodiments, the fibrotic condition of the heart is myocardial fibrosis (e.g., myocardial fibrosis associated with radiation myocarditis, a surgical procedure complication (e.g., myocardial post-operative fibrosis), infectious diseases (e.g., Chagas disease, bacterial, trichinosis or fungal myocarditis)); granulomatous, metabolic storage disorders (e.g., cardiomyopathy, hemochromatosis); developmental disorders (e.g, endocardial fibroelastosis); arteriosclerotic, or exposure to toxins or irritants (e.g., drug induced cardiomyopathy, drug induced cardiotoxicity, alcoholic cardiomyopathy, cobalt poisoning or exposure). In certain embodiments, the myocardial fibrosis is associated with an inflammatory disorder of cardiac tissue (e.g., myocardial sarcoidosis). In some embodiments, the fibrotic condition is a fibrotic condition associated with a myocardial infarction. In some embodiments, the fibrotic condition is a fibrotic condition associated with congestive heart failure.

In some embodiments, the fibrotic condition is associated with an autoimmune disease selected from scleroderma or lupus, e.g., systemic lupus erythematosus.

In some embodiments, the fibrotic condition is systemic. In some embodiments, the fibrotic condition is systemic sclerosis (e.g., limited systemic sclerosis, diffuse systemic sclerosis, or systemic sclerosis sine scleroderma), nephrogenic systemic fibrosis, cystic fibrosis, chronic graft vs. host disease, or atherosclerosis.

In some embodiments, the fibrotic condition is scleroderma. In some embodiments, the scleroderma is localized, e.g., morphea or linear scleroderma. In some embodiments, the condition is a systemic sclerosis, e.g., limited systemic sclerosis, diffuse systemic sclerosis, or systemic sclerosis sine scleroderma.

In other embodiment, the fibrotic condition affects a tissue chosen from one or more of muscle, tendon, cartilage, skin (e.g., skin epidermis or endodermis), cardiac tissue, vascular tissue (e.g., artery, vein), pancreatic tissue, lung tissue, liver tissue, kidney tissue, uterine tissue, ovarian tissue, neural tissue, testicular tissue, peritoneal tissue, colon, small intestine, biliary tract, gut, bone marrow, hematopoietic tissue, or eye (e.g., retinal) tissue.

In some embodiments, the fibrotic condition is a fibrotic condition of the eye. In some embodiments, the fibrotic condition is glaucoma, macular degeneration (e.g., age-related macular degeneration), macular edema (e.g., diabetic macular edema), retinopathy (e.g., diabetic retinopathy), or dry eye disease.

In certain embodiments, the fibrotic condition is a fibrotic condition of the skin. In certain embodiments, the fibrotic condition of the skin is chosen from one or more of: skin fibrosis (e.g., hypertrophic scarring, keloid), scleroderma, nephrogenic systemic fibrosis (e.g., resulting after exposure to gadolinium (which is frequently used as a contrast substance for MRIs) in patients with severe kidney failure), and keloid.

In certain embodiments, the fibrotic condition is a fibrotic condition of the gastrointestinal tract. In certain embodiments, the fibrotic condition is chosen from one or more of: fibrosis associated with scleroderma; radiation induced gut fibrosis; fibrosis associated with a foregut inflammatory disorder such as Barrett's esophagus and chronic gastritis, and/or fibrosis associated with a hindgut inflammatory disorder, such as inflammatory bowel disease (IBD), ulcerative colitis and Crohn's disease. In some embodiments, the fibrotic condition of the gastrointestinal tract is fibrosis associated with scleroderma.

In one embodiment, the fibrotic condition is a chronic fibrotic condition or disorder. In certain embodiments, the fibrotic condition is associated with an inflammatory condition or disorder.

In some embodiments, the fibrotic and/or inflammatory condition is osteomyelitis, e.g., chronic osteomyelitis.

In some embodiments, the fibrotic condition is an amyloidosis. In certain embodiments, the amyloidosis is associated with chronic osteomyelitis.

In some embodiments, the one or more compositions described herein is administered in combination with one or more other therapeutic agents. Exemplary therapeutic agents include, but are not limited to, anti-fibrotics, corticosteroids, antiinflammatories, immunosuppressants, chemotherapeutic agents, anti-metabolites, and immunomodulators.

An example of suitable therapeutics for use in combination with the composition(s) for treatment of liver fibrosis includes, but is not limited to, adefovir dipivoxil, candesartan, colchicine, combined ATG, mycophenolate mofetil, and tacrolimus, combined cyclosporine microemulsion and tacrolimus, elastometry, everolimus, FG-3019, Fuzheng Huayu, GI262570, glycyrrhizin (monoammonium glycyrrhizinate, glycine, L-cysteine monohydrochloride), interferon gamma-1b, irbesartan, losartan, oltipraz, ORAL IMPACT®, peginterferon alfa-2a, combined peginterferon alfa-2a and ribavirin, peginterferon alfa-2b (SCH 54031), combined peginterferon alpha-2b and ribavirin, praziquantel, prazosin, raltegravir, ribavirin (REBETOL®, SCH 18908), ritonavir-boosted protease inhibitor, pentoxyphilline, tacrolimus, tauroursodeoxycholic acid, tocopherol, ursodiol, warfarin, and combinations thereof.

### Combination Therapies

The multispecific molecules disclosed herein can be used in combination with a second therapeutic agent or procedure.

In embodiments, the multispecific molecule and the second therapeutic agent or procedure are administered/performed after a subject has been diagnosed with a cancer, e.g., before the cancer has been eliminated from the subject. In embodiments, the multispecific molecule and the second therapeutic agent or procedure are administered/performed simultaneously or concurrently. For example, the delivery of one treatment is still occurring when the delivery of the second commences, e.g., there is an overlap in administration of the treatments. In other embodiments, the multispecific molecule and the second therapeutic agent or procedure are administered/performed sequentially. For example, the delivery of one treatment ceases before the delivery of the other treatment begins.

In embodiments, combination therapy can lead to more effective treatment than monotherapy with either agent alone. In embodiments, the combination of the first and second treatment is more effective (e.g., leads to a greater reduction in symptoms and/or cancer cells) than the first or second treatment alone. In embodiments, the combination therapy permits use of a lower dose of the first or the second treatment compared to the dose of the first or second treatment normally required to achieve similar effects when administered as a monotherapy. In embodiments, the combination therapy has a partially additive effect, wholly additive effect, or greater than additive effect.

In one embodiment, the multispecific molecule is administered in combination with a therapy, e.g., a cancer therapy (e.g., one or more of anti-cancer agents, immunotherapy, photodynamic therapy (PDT), surgery and/or radiation). The terms “chemotherapeutic,” “chemotherapeutic agent,” and “anti-cancer agent” are used interchangeably herein. The administration of the multispecific molecule and the therapy, e.g., the cancer therapy, can be sequential (with or without overlap) or simultaneous. Administration of the multispecific molecule can be continuous or intermittent during the course of therapy (e.g., cancer therapy). Certain therapies described herein can be used to treat cancers and non-cancerous diseases. For example, PDT efficacy can be enhanced in cancerous and non-cancerous conditions (e.g., tuberculosis) using the methods and compositions described herein (reviewed in, e.g., Agostinis, P. et al. (2011) CA Cancer J. Clin. 61:250-281).

### Anti-Cancer Therapies

In other embodiments, the multispecific molecule is administered in combination with a low or small molecular weight chemotherapeutic agent. Exemplary low or small molecular weight chemotherapeutic agents include, but not limited to, 13-cis-retinoic acid (isotretinoin, ACCUTANE®), 2-CdA (2-chlorodeoxyadenosine, cladribine, LEUSTATIN™), 5-azacitidine (azacitidine, VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANRETIN®), all-transretinoic acid (ATRA, tretinoin, VESANOID®), altretamine (hexamethylmelamine, HMM, HEXALEN®), amethopterin (methotrexate, methotrexate sodium, MTX, TREXALL™, RHEUMATREX®), amifostine (ETHYOL®), arabinosylcytosine (Ara-C, cytarabine, CYTOSAR-U®), arsenic trioxide (TRISENOX®), asparaginase (Erwinia L-asparaginase, L-asparaginase, ELSPAR®, KIDROLASE®), BCNU (carmustine, BiCNU®), bendamustine (TREANDA®), bexarotene (TARGRETIN®), bleomycin (BLENOXANE®), busulfan (BUSULFEX®, MYLERAN®), calcium leucovorin (Citrovorum Factor, folinic acid, leucovorin), camptothecin-11 (CPT-11, irinotecan, CAMPTOSAR®), capecitabine (XELODA®), carboplatin (PARAPLATIN®), carmustine wafer (prolifeprospan 20 with carmustine implant, GLIADEL® wafer), CCI-779 (temsirolimus, TORISEL®), CCNU (lomustine, CeeNU), CDDP (cisplatin, PLATINOL®, PLATINOL-AQ®), chlorambucil (leukeran), cyclophosphamide (CYTOXAN®, NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate, TOPOSAR®, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCIN®), ifosfamide (IFEX®), ixabepilone (IXEMPRA™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™), mitomycin (mitomycin-C, MTC, MUTAMYCIN®), nelarabine (ARRANON®), oxaliplatin (ELOXATIN™), paclitaxel (TAXOL®, ONXAL™), pegaspargase (PEG-L-asparaginase, ONCOSPAR®), PEMETREXED (ALIMTA®), pentostatin (NIPENT®), procarbazine (MATULANE®), streptozocin (ZANOSAR®), temozolomide (TEMODAR®), teniposide (VM-26, VUMON®), TESPA (thiophosphoamide, thiotepa, TSPA, THIOPLEX®), topotecan (HYCAMTIN®), vinblastine (vinblastine sulfate, vincaleukoblastine, VLB, ALKABAN-AQ®, VELBAN®), vinorelbine (vinorelbine tartrate, NAVELBINE®), and vorinostat (ZOLINZA®).

In another embodiment, the multispecific molecule is administered in conjunction with a biologic. Biologics useful in the treatment of cancers are known in the art and a binding molecule of the invention may be administered, for example, in conjunction with such known biologics. For example, the FDA has approved the following biologics for the treatment of breast cancer: HERCEPTIN® (trastuzumab, Genentech Inc., South San Francisco, Calif.; a humanized monoclonal antibody that has anti-tumor activity in HER2-positive breast cancer); FASLODEX® (fulvestrant, AstraZeneca Pharmaceuticals, LP, Wilmington, Del.; an estrogen-receptor antagonist used to treat breast cancer); ARIMIDEX® (anastrozole, AstraZeneca Pharmaceuticals, LP; a nonsteroidal aromatase inhibitor which blocks aromatase, an enzyme needed to make estrogen); Aromasin® (exemestane, Pfizer Inc., New York, N.Y.; an irreversible, steroidal aromatase inactivator used in the treatment of breast cancer); FEMARA® (letrozole, Novartis Pharmaceuticals, East Hanover, N.J.; a nonsteroidal aromatase inhibitor approved by the FDA to treat breast cancer); and NOLVADEX® (tamoxifen, AstraZeneca Pharmaceuticals, LP; a nonsteroidal antiestrogen approved by the FDA to treat breast cancer). Other biologics with which the binding molecules of the invention may be combined include: AVASTIN® (bevacizumab, Genentech Inc.; the first FDA-approved therapy designed to inhibit angiogenesis); and ZEVALIN® (ibritumomab tiuxetan, Biogen Idec, Cambridge, Mass.; a radiolabeled monoclonal antibody currently approved for the treatment of B-cell lymphomas).

In addition, the FDA has approved the following biologics for the treatment of colorectal cancer: AVASTIN®; ERBITUX® (cetuximab, ImClone Systems Inc., New York, N.Y., and Bristol-Myers Squibb, New York, N.Y.; is a monoclonal antibody directed against the epidermal growth factor receptor (EGFR)); GLEEVEC® (imatinib mesylate; a protein kinase inhibitor); and ERGAMISOL® (levamisole hydrochloride, Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For the treatment of lung cancer, exemplary biologics include TARCEVA® (erlotinib HCL, OSI Pharmaceuticals Inc., Melville, N.Y.; a small molecule designed to target the human epidermal growth factor receptor 1 (HER1) pathway).

For the treatment of multiple myeloma, exemplary biologics include VELCADE® Velcade (bortezomib, Millennium Pharmaceuticals, Cambridge Mass.; a proteasome inhibitor). Additional biologics include THALIDOMID® (thalidomide, Clegene Corporation, Warren, N.J.; an immunomodulatory agent and appears to have multiple actions, including the ability to inhibit the growth and survival of myeloma cells and anti-angiogenesis).

Additional exemplary cancer therapeutic antibodies include, but are not limited to, 3F8, abagovomab, adecatumumab, afutuzumab, alacizumab pegol, alemtuzumab (CAMPATH®, MABCAMPATH®), altumomab pentetate (HYBRI-CEAKER®), anatumomab mafenatox, anrukinzumab (IMA-638), apolizumab, arcitumomab (CEA-SCAN®), bavituximab, bectumomab (LYMPHOSCAN®), belimumab (BENLYSTA®, LYMPHOSTAT-B®), besilesomab (SCINTIMUN®), bevacizumab (AVASTIN®), bivatuzumab mertansine, blinatumomab, brentuximab vedotin, cantuzumab mertansine, capromab pendetide (PROSTASCINT®), catumaxomab (REMOVAB®), CC49, cetuximab (C225, ERBITUX®), citatuzumab bogatox, cixutumumab, clivatuzumab tetraxetan, conatumumab, dacetuzumab, denosumab (PROLIA®), detumomab, ecromeximab, edrecolomab (PANOREX®), elotuzumab, epitumomab cituxetan, epratuzumab, ertumaxomab (REXOMUN®), etaracizumab, farletuzumab, figitumumab, fresolimumab, galiximab, gemtuzumab ozogamicin (MYLOTARG®), girentuximab, glembatumumab vedotin, ibritumomab (ibritumomab tiuxetan, ZEVALIN®), igovomab (INDIMACIS-125®), intetumumab, inotuzumab ozogamicin, ipilimumab, iratumumab, labetuzumab (CEA-CIDE®), lexatumumab, lintuzumab, lucatumumab, lumiliximab, mapatumumab, matuzumab, milatuzumab, minretumomab, mitumomab, nacolomab tafenatox, naptumomab estafenatox, necitumumab, nimotuzumab (THERACIM®, THERALOC®), nofetumomab merpentan (VERLUMA®), ofatumumab (ARZERRA®), olaratumab, oportuzumab monatox, oregovomab (OVAREX®), panitumumab (VECTIBIX®), pemtumomab (THERAGYN®), pertuzumab (OMNITARG®), pintumomab, pritumumab, ramucirumab, ranibizumab (LUCENTIS®), rilotumumab, rituximab (MABTHERA®, RITUXAN®), robatumumab, satumomab pendetide, sibrotuzumab, siltuximab, sontuzumab, tacatuzumab tetraxetan (AFP-CIDE®), taplitumomab paptox, tenatumomab, TGN1412, ticilimumab (tremelimumab), tigatuzumab, TNX-650, tositumomab (BEXXAR®), trastuzumab (HERCEPTIN®), tremelimumab, tucotuzumab celmoleukin, veltuzumab, volociximab, votumumab (HUMASPECT®), zalutumumab (HUMAX-EGFR®), and zanolimumab (HUMAX-CD4®).

In other embodiments, the multispecific molecule is administered in combination with a viral cancer therapeutic agent. Exemplary viral cancer therapeutic agents include, but not limited to, vaccinia virus (vvDD-CDSR), carcinoembryonic antigen-expressing measles virus, recombinant vaccinia virus (TK-deletion plus GM-CSF), Seneca Valley virus-001, Newcastle virus, coxsackie virus A21, GL-ONC1, EBNA1 C-terminal/LMP2 chimeric protein-expressing recombinant modified vaccinia Ankara vaccine, carcinoembryonic antigen-expressing measles virus, G207 oncolytic virus, modified vaccinia virus Ankara vaccine expressing p53, OncoVEX GM-CSF modified herpes-simplex 1 virus, fowlpox virus vaccine vector, recombinant vaccinia prostate-specific antigen vaccine, human papillomavirus 16/18 L1 virus-like particle/ASO4 vaccine, MVA-EBNA1/LMP2 Inj. vaccine, quadrivalent HPV vaccine, quadrivalent human papillomavirus (types 6, 11, 16, 18) recombinant vaccine (GARDASIL®), recombinant fowlpox-CEA(6D)/TRICOM vaccine; recombinant vaccinia-CEA(6D)-TRICOM vaccine, recombinant modified vaccinia Ankara-5T4 vaccine, recombinant fowlpox-TRICOM vaccine, oncolytic herpes virus NV1020, HPV L1 VLP vaccine V504, human papillomavirus bivalent (types 16 and 18) vaccine (CERVARIX®), herpes simplex virus HF10, Ad5CMV-p53 gene, recombinant vaccinia DF3/MUC1 vaccine, recombinant vaccinia-MUC-1 vaccine, recombinant vaccinia-TRICOM vaccine, ALVAC MART-1 vaccine, replication-defective herpes simplex virus type I (HSV-1) vector expressing human Preproenkephalin (NP2), wild-type reovirus, reovirus type 3 Dearing (REOLYSIN®), oncolytic virus HSV1716, recombinant modified vaccinia Ankara (MVA)-based vaccine encoding Epstein-Barr virus target antigens, recombinant fowlpox-prostate specific antigen vaccine, recombinant vaccinia prostate-specific antigen vaccine, recombinant vaccinia-B7.1 vaccine, rAd-p53 gene, Ad5-delta24RGD, HPV vaccine 580299, JX-594 (thymidine kinase-deleted vaccinia virus plus GM-CSF), HPV-16/18 L1/AS04, fowlpox virus vaccine vector, vaccinia-tyrosinase vaccine, MEDI-517 HPV-16/18 VLP AS04 vaccine, adenoviral vector containing the thymidine kinase of herpes simplex virus TK99UN, HspE7, FP253/Fludarabine, ALVAC(2) melanoma multi-antigen therapeutic vaccine, ALVAC-hB7.1, canarypox-hIL-12 melanoma vaccine, Ad-REIC/Dkk-3, rAd-IFN SCH 721015, TIL-Ad-INFg, Ad-ISF35, and coxsackievirus A21 (CVA21, CAVATAK®).

In other embodiments, the multispecific molecule is administered in combination with a nanopharmaceutical. Exemplary cancer nanopharmaceuticals include, but not limited to, ABRAXANE® (paclitaxel bound albumin nanoparticles), CRLX101 (CPT conjugated to a linear cyclodextrin-based polymer), CRLX288 (conjugating docetaxel to the biodegradable polymer poly (lactic-co-glycolic acid)), cytarabine liposomal (liposomal Ara-C, DEPOCYT™) daunorubicin liposomal (DAUNOXOME®), doxorubicin liposomal (DOXIL®, CAELYX®), encapsulated-daunorubicin citrate liposome (DAUNOXOME®), and PEG anti-VEGF aptamer (MACUGEN®).

In some embodiments, the multispecific molecule is administered in combination with paclitaxel or a paclitaxel formulation, e.g., TAXOL®, protein-bound paclitaxel (e.g., ABRAXANE®). Exemplary paclitaxel formulations include, but are not limited to, nanoparticle albumin-bound paclitaxel (ABRAXANE®, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620).

Exemplary RNAi and antisense RNA agents for treating cancer include, but not limited to, CALAA-01, siG12D LODER (Local Drug EluteR), and ALN-VSP02.

Other cancer therapeutic agents include, but not limited to, cytokines (e.g., aldesleukin (IL-2, Interleukin-2, PROLEUKIN®), alpha Interferon (IFN-alpha, Interferon alfa, INTRON® A (Interferon alfa-2b), ROFERON-A® (Interferon alfa-2a)), Epoetin alfa (PROCRIT®), filgrastim (G-CSF, Granulocyte—Colony Stimulating Factor, NEUPOGEN®), GM-CSF (Granulocyte Macrophage Colony Stimulating Factor, sargramostim, LEUKINE™), IL-11 (Interleukin-11, oprelvekin, NEUMEGA®), Interferon alfa-2b (PEG conjugate) (PEG interferon, PEG-INTRON™), and pegfilgrastim (NEULASTA™)), hormone therapy agents (e.g., aminoglutethimide (CYTADREN®), anastrozole (ARIMIDEX®), bicalutamide (CASODEX®), exemestane (AROMASIN®), fluoxymesterone (HALOTESTIN®), flutamide (EULEXIN®), fulvestrant (FASLODEX®), goserelin (ZOLADEX®), letrozole (FEMARA®), leuprolide (ELIGARD™, LUPRON®, LUPRON DEPOT®, VIADUR™), megestrol (megestrol acetate, MEGACE®), nilutamide (ANANDRON®, NILANDRON®), octreotide (octreotide acetate, SANDOSTATIN®, SANDOSTATIN LAR®), raloxifene (EVISTA®), romiplostim (NPLATE®), tamoxifen (NOVALDEX®), and toremifene (FARESTON®)), phospholipase A2 inhibitors (e.g., anagrelide (AGRYLIN®)), biologic response modifiers (e.g., BCG (THERACYS®, TICE®), and Darbepoetin alfa (ARANESP®)), target therapy agents (e.g., bortezomib (VELCADE®), dasatinib (SPRYCEL™), denileukin diftitox (ONTAK®), erlotinib (TARCEVA®), everolimus (AFINITOR®), gefitinib (TRESSA®), imatinib mesylate (STI-571, GLEEVEC™), lapatinib (TYKERB®), sorafenib (NEXAVAR®), and SU11248 (sunitinib, SUTENT®)), immunomodulatory and antiangiogenic agents (e.g., CC-5013 (lenalidomide, REVLIMID®), and thalidomide (THALOMID®)), glucocorticosteroids (e.g., cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL®), prednisolone (DELTA-CORTEF®, ORAPRED®, PEDIAPRED®, PRELONE®), and prednisone (DELTASONE®, LIQUID PRED®, METICORTEN®, ORASONE®)), and bisphosphonates (e.g., pamidronate (AREDIA®), and zoledronic acid (ZOMETA®))

In some embodiments, the multispecific molecule is used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., an antibody against VEGF, a VEGF trap, a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-8 inhibitor)), a RAF-1 inhibitor, a KIT inhibitor and a RET inhibitor. In some embodiments, the anti-cancer agent used in combination with the multispecific molecule is selected from the group consisting of: axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951(tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, Pazopanib Hydrochloride, PD173074, nSorafenib Tosylate(Bay 43-9006), SU 5402, TSU-68(SU6668), vatalanib, XL880 (GSK1363089, EXEL-2880). Selected tyrosine kinase inhibitors are chosen from sunitinib, erlotinib, gefitinib, or sorafenib. In one embodiment, the tyrosine kinase inhibitor is sunitinib.

In one embodiment, the multispecific molecule is administered in combination with one of more of: an anti-angiogenic agent, or a vascular targeting agent or a vascular disrupting agent. Exemplary anti-angiogenic agents include, but are not limited to, VEGF inhibitors (e.g., anti-VEGF antibodies (e.g., bevacizumab); VEGF receptor inhibitors (e.g., itraconazole); inhibitors of cell proliferatin and/or migration of endothelial cells (e.g., carboxyamidotriazole, TNP-470); inhibitors of angiogenesis stimulators (e.g., suramin), among others. A vascular-targeting agent (VTA) or vascular disrupting agent (VDA) is designed to damage the vasculature (blood vessels) of cancer tumors causing central necrosis (reviewed in, e.g., Thorpe, P. E. (2004) Clin. Cancer Res. Vol. 10:415-427). VTAs can be small-molecule. Exemplary small-molecule VTAs include, but are not limited to, microtubule destabilizing drugs (e.g., combretastatin A-4 disodium phosphate (CA4P), ZD6126, AVE8062, Oxi 4503); and vadimezan (ASA404).

### Immune Checkpoint Inhibitors

In other embodiments, methods described herein comprise use of an immune checkpoint inhibitor in combination with the multispecific molecule. The methods can be used in a therapeutic protocol in vivo.

In embodiments, an immune checkpoint inhibitor inhibits a checkpoint molecule. Exemplary checkpoint molecules include but are not limited to CTLA4, PD1, PD-L1, PD-L2, TIM3, LAG3, CD160, 2B4, CD80, CD86, B7-H3 (CD276), B7-H4 (VTCN1), HVEM (TNFRSFi4 or CD270), BTLA, KIR, MHC class I, MHC class II, GAL9, VISTA, BTLA, TIGIT, LAIR1, and A2aR. See, e.g., Pardoll. Nat. Rev. Cancer 12.4(2012):252-64, incorporated herein by reference.

In embodiments, the immune checkpoint inhibitor is a PD-1 inhibitor, e.g., an anti-PD-1 antibody such as Nivolumab, Pembrolizumab or Pidilizumab. Nivolumab (also called MDX-1106, MDX-1106-04, ONO-4538, or BMS-936558) is a fully human IgG4 monoclonal antibody that specifically inhibits PD1. See, e.g., U.S. Pat. No. 8,008,449 and WO2006/121168. Pembrolizumab (also called Lambrolizumab, MK-3475, MK03475, SCH-900475 or KEYTRUDA®; Merck) is a humanized IgG4 monoclonal antibody that binds to PD-1. See, e.g., Hamid, O. et al. (2013) New England Journal of Medicine 369 (2): 134-44, U.S. Pat. No. 8,354,509 and WO2009/114335. Pidilizumab (also called CT-011 or Cure Tech) is a humanized IgGik monoclonal antibody that binds to PD1. See, e.g., WO2009/101611. In one embodiment, the inhibitor of PD-1 is an antibody molecule having a sequence substantially identical or similar thereto, e.g., a sequence at least 85%, 90%, 95% identical or higher to the sequence of Nivolumab, Pembrolizumab or Pidilizumab. Additional anti-PD1 antibodies, e.g., AMP 514 (Amplimmune), are described, e.g., in U.S. Pat. No. 8,609,089, US 2010028330, and/or US 20120114649.

In some embodiments, the PD-1 inhibitor is an immunoadhesin, e.g., an immunoadhesin comprising an extracellular/PD-1 binding portion of a PD-1 ligand (e.g., PD-L1 or PD-L2) that is fused to a constant region (e.g., an Fc region of a heavy chain). In embodiments, the PD-1 inhibitor is AMP-224 (B7-DCIg, e.g., described in WO2011/066342 and WO2010/027827), a PD-L2 Fc fusion soluble receptor that blocks the interaction between B7-H1 and PD-1.

In embodiments, the immune checkpoint inhibitor is a PD-L1 inhibitor, e.g., an antibody molecule. In some embodiments, the PD-L1 inhibitor is YW243.55.S70, MPDL3280A, MEDI-4736, MSB-0010718C, or MDX-1105. In some embodiments, the anti-PD-L1 antibody is MSB0010718C (also called A09-246-2; Merck Serono), which is a monoclonal antibody that binds to PD-L1. Exemplary humanized anti-PD-L1 antibodies are described, e.g., in WO2013/079174. In one embodiment, the PD-L1 inhibitor is an anti-PD-L1 antibody, e.g., YW243.55.S70. The YW243.55.S70 antibody is described, e.g., in WO 2010/077634. In one embodiment, the PD-L1 inhibitor is MDX-1105 (also called BMS-936559), which is described, e.g., in WO2007/005874. In one embodiment, the PD-L1 inhibitor is MDPL3280A (Genentech/Roche), which is a human Fc-optimized IgG1 monoclonal antibody against PD-L1. See, e.g., U.S. Pat. No. 7,943,743 and U.S Publication No.: 20120039906. In one embodiment, the inhibitor of PD-L1 is an antibody molecule having a sequence substantially identical or similar thereto, e.g., a sequence at least 85%, 90%, 95% identical or higher to the sequence of YW243.55.S70, MPDL3280A, MEDI-4736, MSB-0010718C, or MDX-1105.

In embodiments, the immune checkpoint inhibitor is a PD-L2 inhibitor, e.g., AMP-224 (which is a PD-L2 Fc fusion soluble receptor that blocks the interaction between PD1 and B7-H1. See, e.g., WO2010/027827 and WO2011/066342.

In one embodiment, the immune checkpoint inhibitor is a LAG-3 inhibitor, e.g., an anti LAG-3 antibody molecule. In embodiments, the anti-LAG-3 antibody is BMS-986016 (also called BMS986016; Bristol-Myers Squibb). BMS-986016 and other humanized anti-LAG-3 antibodies are described, e.g., in US 2011/0150892, WO2010/019570, and WO2014/008218.

In embodiments, the immune checkpoint inhibitor is a TIM-3 inhibitor, e.g., anti-TIM3 antibody molecule, e.g., described in U.S. Pat. No. 8,552,156, WO 2011/155607, EP 2581113 and U.S Publication No.: 2014/044728.

In embodiments, the immune checkpoint inhibitor is a CTLA-4 inhibitor, e.g., anti-CTLA-4 antibody molecule. Exemplary anti-CTLA4 antibodies include Tremelimumab (IgG2 monoclonal antibody from Pfizer, formerly known as ticilimumab, CP-675,206); and Ipilimumab (also called MDX-010, CAS No. 477202-00-9). Other exemplary anti-CTLA-4 antibodies are described, e.g., in U.S. Pat. No. 5,811,097.

### EXAMPLES

The following examples are intended to be illustrative, and are not meant in any way to be limiting.

### Example 1. Generation of Multiple αCCR2/αCSF1R Bispecific Antibody Molecules

### 1. Construction of the Plasmids.

The DNA encoding the protein sequences was optimized for expression in Cricetulus griseus, synthesized, and cloned into the pcDNA3.4-TOPO (Life Technologies A14697) using Gateway cloning. All constructs contained an Ig Kappa leader sequence (ATGGAAACCGACACACTGCTGCTGTGGGTGCTGCTCTTGTGGGTGCCAGGATCTAC AGGA (SEQ ID NO: 115), METDTLLLWVLLLWVPGSTG (SEQ ID NO: 116)). The nucleic acid sequences used are shown in Table 1.

TABLE 1
Exemplary nucleic acid sequences of antibodies

| SEQ ID | | |
|---|---|---|
| NO | Description | Nucleic Acid Sequence |
| SEQ ID | αCCR2 | CAGGTCCAGCTGCAAGAGTCTGGCCCTGGACTGGTTCAGCCCTCTCAGA |
| NO: 1 | MC12 VH | CCCTGTCTCTGACCTGTACCGTGTCCGGCTTCTCCCTGACCGACTTCTCTG |
| | | TGCACTGGGTCCGACAGCCTCCAGGCAAAGGACTGGAATGGATGGGCAG |
| | | AATCAGATCCGAGGGCAACACCGACTACAACAGCGCCCTGAAGTCCCGG |
| | | CTGTCTATCAGCAGAGACACCTCCAAGAGCCAGGTGTTCCTGAAGATGA |
| | | ACTCCCTGCAGACCGAGGACACCGCCATCTATTTCTGCACCAGAGGCGA |
| | | CATCCTCGGCTTCGGCTATTGGGGACAGGGCGTGATGGTCACCGTTAGCT |
| | | CT |
| SEQ ID | αCCR2 | GACATCGTGATGACCCAGTCTCCACTGTCCGTGTCTGTGACCCCTGGCGA |
| NO: 2 | MC12 VL | GTCTGCCTCCATCTCCTGCAGATCCTCCAAGAGCCTGCTGCACTTCAAGG |
| | | GCATCACCTTCGTGTACTGGTATCTGCAGAAGCCCGGCCAGTCTCCTCAG |
| | | CTGCTGATCTTCAGAATGTCCAGCCTGGCCTCTGGCGTGCCCGATAGATT |
| | | TTCTGGCTCCGGCTCCGAGACAGACTTCACCCTGAAGATCTCCAGAGTG |
| | | GAAGCCGAGGACGTGGGCACCTACTATTGTGGCCAGCTGCTGGAAAACC |
| | | CCTACACCTTTGGCGCTGGCACCAAGCTGGAACTGAAG |
| SEQ ID | R2b CH1 | GCTCAGACCACCGCTCCTAGCGTGTACCCTTTGGCTCCTGGCTGTGGCGA |
| NO: 3 | | CACCACCTCTTCTACAGTGACCCTGGGCTGTCTGGTCAAGGGCTACTTTC |
| | | CTGAGCCTGTGACCGTGACCTGGAACTCTGGTGCCCTGTCCTCCGACGTG |
| | | CACACCTTTCCAGCTGTGCTGCAGTCCGGCCTGTACACCCTGACATCCTC |
| | | CGTGACCTCTTCCACCTGGCCTAGCCAGACCGTGACATGCAATGTGGCTC |
| | | ACCCTGCCTCCAGCACCAAGGTGGACAAGAAGGTGGAACGGCGG |
| SEQ ID | R2b CL | AGAGCTGACGCTGCCCCTACCGTGTCTATCTTCCCTCCATCCATGGAACA |
| NO: 4 | | GCTGACCTCTGGCGGAGCTACCGTCGTGTGCTTCGTGAACAACTTCTACC |
| | | CTCGGGACATCTCCGTGAAGTGGAAGATCGACGGCTCTGAGCAGCGAGA |
| | | TGGCGTGCTGGATTCTGTGACCGACCAGGACTCCAAGGACAGCACCTAC |
| | | TCCATGTCTAGCACCCTGAGCCTGACCAAGGTGGAATACGAGCGGCACA |
| | | ACCTGTATACCTGCGAGGTGGTGCACAAGACCTCCAGCTCTCCCGTGGTC |
| | | AAGTCCTTCAACCGGAACGAGTGC |
| SEQ ID | αmCSF1R | CAGGTCCAGTTGCAGCAGTCTGGCGCTGAGCTGGTCAAGCCTGGATCCT |
| NO: 5 | VH | CCGTGAAGATCTCCTGCAAGGCCTCCGGCTACACCTTCACCTCCAACTTC |
| | | ATGCACTGGATCAAGCAGCAGCCCGGCAACGGCCTGGAATGGATCGGAT |
| | | GGATCTATCCTGGCGACGGCGACACCGAGTACAACCAGAAGTTCAACGG |
| | | CAAGGCTACCCTGACCGCCGACAAGTCCTCTTCCACCGCTTACATGCAGC |
| | | TGTCCAGCCTGACCTCTGAGGACTCCGCCGTGTACTTCTGCGCCGTGAAT |
| | | TATGGCGGCTACGTGCTGGATGCTTGGGGCCAAGGCGCTTCTGTGACAG |
| | | TGTCCTCT |
| SEQ ID | R2a CH1 | GCCGAGACAACCGCTCCTAGCGTTTACCCTCTGGCTCCTGGCACAGCCCT |
| NO: 6 | | GAAGTCCAACTCTATGGTCACCCTGGGCTGCCTGGTCAAGGGCTACTTTC |
| | | CTGAGCCTGTGACCGTGACCTGGAACTCTGGTGCTCTGTCTAGCGGCGTG |
| | | CACACCTTTCCAGCTGTGCTGCAGAGCGGCCTGTACACCCTGACATCTAG |
| | | CGTGACCGTGCCTTCCAGCACCTGGTCTAGTCAGGCTGTGACCTGCAACG |
| | | TGGCCCATCCTGCCTCTTCTACCAAGGTGGACAAGAAAATCGTGCCCAG |
| | | AGAGTGCAAC |
| SEQ ID | αmCSF1R | GAGATCGTGCTGACCCAGTCTCCTACCACCATGGCTGCTAGCCCTGGCG |
| NO: 7 | VL | AGAAAGTGACAATTACCTGCCGGGCCTCCTCCTCCACCAACTACATGTCC |
| | | TGGTATCAGCAGAAGTCCGGCGCCTCTCCTAAGCCTTGGATCTACGAGA |
| | | CATCCAAGCTGGCCTCTGGCGTGCCCGATAGATTTTCCGGCTCTGGCTCC |
| | | GGCACCTCCTACAGCTTCACCATCTCCAGCATGGAAACAGAGGACGCCG |
| | | CCACCTACTACTGCCACCAGTGGTCATCTACCCCTCTGACCTTTGGCAGC |
| | | GGCACCAAGCTGGAAATCAAG |
| SEQ ID | R2a CL | AGAGCTGACGCCGCTCCTACCGTGTCTATCTTCCCTCCATCCATGGAACA |
| NO: 8 | | GCTGACCTCCGGCGGAGCTACCGTCGTGTGTTTCGTGAACAACTTCTACC |
| | | CTCGGGACATCTCCGTGAAGTGGAAGATCGACGGCTCTGAGCAGCGAGA |
| | | TGGCGTGCTGGATTCTGTGACCGACCAGGACTCCAAGGACAGCACCTAC |
| | | TCCATGTCTAGCACCCTGAGCCTGACCAAGGTGGAATACGAGCGGCACA |
| | | ACCTGTATACCTGCGAGGTGGTGCACAAGACCTCCAGCTCTCCCGTGGTC |
| | | AAGTCCTTCAACCGGAACGAGTGC |
| SEQ ID | mFc Knob | ACCATTAAGCCTTGTCCTCCATGCAAGTGCCCCGCTCCTAATCTGCTCGG |
| NO: 9 | | AGGCCCTTCCGTGTTCATCTTTCCACCTAAGATCAAGGACGTGCTGATGA |
| | | TCTCCCTGTCTCCTATCGTGACCTGCGTGGTGGTGGACGTGTCCGAGGAT |
| | | GATCCTGACGTGCAGATCAGTTGGTTCGTGAACAACGTGGAAGTGCACA |
| | | CCGCTCAGACCCAGACACACAGAGAGGACTACAACTCTACCCTGAGAGT |
| | | GGTGTCTGCCCTGCCTATCCAGCATCAGGACTGGATGTCCGGCAAAGAA |
| | | TTCAAGTGCAAAGTGAACAACAAGGACCTGCCTGCTCCAATCGAGCGGA |
| | | CCATCTCTAAGCCTAAGGGCTCTGTCAGGGCCCCTCAGGTGTACGTTCTG |
| | | CCTCCTTGCGAGGAAGAGATGACCAAGAAACAAGTGACACTGTGGTGCA |
| | | TGGTCACAGACTTCATGCCCGAGGACATCTACGTGGAATGGACCAACAA |
| | | CGGCAAGACCGAGCTGAACTACAAGAACACCGAGCCTGTGCTGGACTCC |
| | | GACGGCTCCTACTTCATGTACTCCAAGCTGCGCGTCGAGAAGAAGAACT |
| | | GGGTCGAGAGAAACTCCTACTCCTGCTCCGTGGTGCACGAGGGCCTGCA |
| | | CAATCACCACACCACCAAGTCCTTCTCTCGGACCCCTGGCAAG |
| SEQ ID | mFc Hole | ACCATCAAGCCCTGTCCTCCATGCAAGTGCCCCGCTCCTAATCTGCTCGG |
| NO: 10 | | AGGCCCTTCCGTGTTCATCTTCCCACCTAAGATCAAGGACGTGCTGATGA |
| | | TCTCCCTGTCTCCTATCGTGACCTGCGTGGTGGTGGACGTGTCCGAGGAT |
| | | GATCCTGACGTGCAGATCAGTTGGTTCGTGAACAACGTGGAAGTGCACA |
| | | CCGCTCAGACCCAGACACACAGAGAGGACTACAACAGCACCCTGAGAG |
| | | TGGTGTCTGCCCTGCCAATCCAGCACCAGGATTGGATGTCCGGCAAAGA |
| | | ATTCAAGTGCAAAGTGAACAACAAGGACCTGCCTGCTCCAATCGAGCGG |
| | | ACCATCTCTAAGCCTAAGGGCTCTGTGCGGGCTCCCCAAGTTTGTGTTCT |
| | | GCCTCCACCTGAGGAAGAGATGACCAAGAAACAAGTGACCCTGTCTTGT |
| | | GCCGTGACCGACTTCATGCCCGAGGACATCTACGTGGAATGGACCAACA |
| | | ATGGCAAGACCGAGCTGAACTACAAGAACACCGAGCCTGTGCTGGACTC |
| | | CGACGGCTCCTACTTCATGGTGTCTAAGCTGCGCGTCGAGAAGAAGAAC |
| | | TGGGTCGAGAGAAACTCCTACTCCTGCTCCGTGGTGCACGAGGGCCTGC |
| | | ACAATCACCACACCACCAAGTCCTTCTCTCGGACCCCTGGCAAG |
| SEQ ID | αhCCR2 | GAGGTGCAGCTGGTTGAATCTGGCGGAGGACTGGTTAAGCCTGGCGGCT |
| NO: 11 | plozalizumab | CTCTGAGACTGTCTTGTGCCGCTTCTGGCTTCACCTTCTCCGCCTACGCCA |
| | VH | TGAACTGGGTCCGACAGGCTCCTGGCAAAGGCCTGGAATGGGTCGGAAG |
| | | AATCCGGACCAAGAACAACAACTACGCCACCTACTACGCCGACTCCGTG |
| | | AAGGACCGGTTCACCATCTCTCGGGACGACTCCAAGAACACCCTGTACC |
| | | TGCAGATGAACTCCCTGAAAACCGAGGACACCGCCGTGTACTACTGCAC |
| | | CACCTTCTACGGCAATGGCGTGTGGGGACAGGGCACACTGGTTACCGTT |
| | | TCTTCCGCCTCCACCAAGGGACCCTCTGTGTTTCCTCTGGCTCCCTCCAG |
| | | CAAGTCTACCTCTGGTGGAACAGCTGCCCTGGGCTGCCTGGTCAAGGAT |
| | | TACTTTCCTGAGCCTGTGACCGTGTCCTGG |
| SEQ ID | hCH1 | GCTTCTACCAAGGGACCCAGCGTGTTCCCTCTGGCTCCTTCCAGCAAGTC |
| NO: 12 | | TACCTCTGGCGGAACAGCTGCTCTGGGCTGCCTGGTCAAGGACTACTTTC |
| | | CTGAGCCTGTGACCGTGTCTTGGAACTCTGGCGCTCTGACATCCGGCGTG |
| | | CACACATTTCCAGCTGTGCTGCAGTCCTCCGGCCTGTACTCTCTGTCCTCT |
| | | GTCGTGACCGTGCCTTCCAGCTCTCTGGGAACCCAGACCTACATCTGCAA |
| | | TGTGAACCACAAGCCTTCCAACACCAAGGTGGACAAGAGAGTGGAACCC |
| | | AAGTCCTGC |
| SEQ ID | hFc Knob | GATAAGACCCACACATGTCCTCCATGCCCTGCCCCTGAGCTGCTGGGCG |
| NO: 13 | | GACCTTCCGTGTTCCTGTTCCCTCCAAAGCCCAAGGACACCCTGATGATC |
| | | AGCCGGACCCCTGAAGTGACCTGCGTGGTGGTGGATGTGTCCCACGAGG |
| | | ATCCCGAAGTGAAGTTCAATTGGTACGTGGACGGCGTGGAAGTGCACAA |
| | | CGCCAAGACCAAGCCCAGAGAGGAACAGTACAACAGCACCTACCGGGT |
| | | GGTGTCCGTGCTGACCGTGCTGCATCAGGACTGGCTGAACGGCAAAGAG |
| | | TACAAGTGCAAGGTGTCCAACAAGGCCCTGCCTGCCCCTATCGAGAAAA |
| | | CCATCAGCAAGGCCAAGGGCCAGCCCCGCGAACCTCAGGTGTACACACT |
| | | GCCTCCCTGCCGGGAAGAGATGACCAAGAACCAGGTGTCCCTGTGGTGC |
| | | CTGGTCAAGGGCTTCTACCCCTCCGATATCGCCGTGGAATGGGAGAGCA |
| | | ACGGCCAGCCCGAGAACAACTACAAGACCACCCCTCCCGTGCTGGACAG |
| | | CGACGGCAGCTTCTTCCTGTACTCCAAACTGACCGTGGACAAGAGCCGG |
| | | TGGCAGCAGGGCAATGTGTTCAGCTGTAGCGTGATGCACGAGGCCCTGC |
| | | ACAACCACTACACCCAGAAGTCCCTGAGCCTGTCTCCTGGCAAA |
| SEQ ID | αhCCR2 | GACGTGGTCATGACACAGAGCCCTCTGTCTCTGCCCGTGACATTGGGAC |
| NO: 14 | plozalizumab | AGCCTGCCTCCATCTCCTGCAAGTCCTCTCAGTCCCTGCTGGACTCTGAC |
| | VL | GGCAAGACCTTCCTGAACTGGTTCCAGCAGCGGCCTGGCCAGTCTCCTA |
| | | GAAGGCTGATCTACCTGGTGTCCAAGCTGGATTCTGGCGTGCCCGACAG |
| | | ATTCTCCGGCTCTGGCTCTGGCACCGACTTCACCCTGAAGATCTCCAGAG |
| | | TGGAAGCCGAGGACGTGGGCGTGTACTACTGTTGGCAGGGCACCCACTT |
| | | TCCATACACCTTCGGCCAGGGCACCAGACTGGAAATCAAG |
| SEQ ID | hCL (kappa) | AGAACAGTGGCCGCTCCTTCCGTGTTCATCTTCCCACCCTCCGACGAGCA |
| NO: 15 | | GCTGAAGTCCGGCACCGCTTCTGTCGTGTGCCTGCTCAACAACTTCTACC |
| | | CTCGGGAAGCCAAGGTGCAGTGGAAGGTGGACAACGCCCTGCAGTCCG |
| | | GCAACTCCCAGGAATCCGTCACCGAGCAGGACTCCAAGGACAGCACCTA |
| | | CTCCCTGTCCTCCACCCTGACCCTGTCCAAGGCCGACTACGAGAAGCAC |
| | | AAGGTGTACGCCTGCGAAGTGACCCACCAGGGCCTGAGCAGCCCCGTGA |
| | | CCAAGTCCTTCAACCGGGGCGAGTGC |
| SEQ ID | αhCCR2 D1 | GAAGTGCAGCTGGTTCAGTCTGGCGCCGAAGTGAAGAAACCTGGCGCCT |
| NO: 16 | VH | CTGTGAAGGTGTCCTGCAAGGCTTCTGGCTACACCTTTACCGGCTACCAC |
| | | ATGCACTGGGTCCGACAGGCTCCAGGACAAGGCTTGGAATGGATGGGCT |
| | | GGATCAACCCCAACTCCGGCGTGACCAAATACGCCCAGAAATTCCAGGG |
| | | CAGAGTGACCATGACCAGAGACACCTCCATCAACACCGCCTACATGGAA |
| | | CTGTCCCGGCTGAGATTCGACGACACCGACGTGTACTACTGTGCCACCG |
| | | GCGGCTTTGGCTATTGGGGAGAGGGAACACTGGTCACCGTGTCCTCC |
| SEQ ID | αhCCR2 D1 | CTGCCCGTGTTGACCCAGCCTCCTAGCGTTTCCAAGGGCCTGAGACAGA |
| NO: 17 | VL | CCGCCACACTGACCTGTACCGGCAACTCTAACAACGTGGGCAATCAGGG |
| | | CGCTGCCTGGTTGCAGCAGCATCAGGGACAGCCTCCAAAGCTGCTGTCC |
| | | TACCGGAACCACAACAGACCTAGCGGCGTGTCCGAGCGGTTCAGCCCTT |
| | | CTAGATCTGGCGACACCTCCAGCCTGACCATCACTGGACTGCAGCCTGA |
| | | GGACGAGGCCGACTACTATTGTCTGGCCTGGGACAGCTCCCTGCGGGCC |
| | | TTTGTTTTTGGCACCGGCACCAAGCTGACCGTGCTG |
| SEQ ID | hCL | GGACAACCTAAGGCCAATCCTACCGTGACACTGTTCCCTCCATCCTCCGA |
| NO: 18 | (lambda) | GGAACTGCAGGCCAACAAGGCTACCCTCGTGTGCCTGATCTCCGACTTTT |
| | | ACCCTGGCGCTGTGACCGTGGCCTGGAAGGCTGATGGATCTCCTGTGAA |
| | | GGCTGGCGTGGAAACCACCAAGCCTTCCAAGCAGTCCAACAACAAATAC |
| | | GCCGCCTCCTCCTACCTGTCTCTGACCCCTGAACAGTGGAAGTCCCACCG |
| | | GTCCTACAGCTGCCAAGTGACCCATGAGGGCTCCACCGTGGAAAAGACC |
| | | GTGGCTCCTACCGAGTGCTCC |
| SEQ ID | αhCCR2 | CAGGTGCAGCTGGTTCAGTCTGGCGCCGAAGTGAAGAAACCTGGCGCCT |
| NO: 19 | 42G7 VH | CTGTGAAGGTGTCCTGCAAGGCTTCCGGCTACACCTTCTCCAGCTACTAC |
| | | ATGCACTGGGTCCGACAGGCCCCTGGACAAGGATTGGAGTGGATGGGCA |
| | | TCATCAACCCCTCTGGCGGCAACACCTCTTACGCCCAGAAATTCCAGGG |
| | | CAGAGTGACCATGACCAGAGACACCTCCACCAGCACCGTGTACATGGAA |
| | | CTGTCCAGCCTGAGATCCGAGGACACCGCCGTGTACTACTGTGCCAGAG |
| | | GCGGATACCAGCTGCCTCACGGTAGAGCCAGAGCCTTCGATATGTGGGG |
| | | CCAGGGCACAATGGTCACCGTGTCCTCT |
| SEQ ID | αhCCR2 | GCCATCAGAATGACCCAGTCTCCACTGAGCCTGCCTGTGACATTGGGCC |
| NO: 20 | 42G7 VL | AGCCTGCCTCTATCTCCTGCACCTCCTCTCAGTCTCTGGTGTACAGAGAT |
| | | GGCACCACCTACCTGAACTGGTTCCAGCAGAGGCCTGGCCAGTCTCCTA |
| | | GACGGCTGATCTACAAGGTGTCCAACAGAGACTCTGGCGTGCCCGACAG |
| | | ATTCACCGGCTCTGGCTCTGGCACCACATTCACCCTGACCATCTCCAGAG |
| | | TGGAAGCCGAGGACGTGGGCATCTACTACTGTATGCAGGGCACCCACTG |
| | | GCCTCTGACCTTTGGCCAGGGAACAAAGGTGGAAATCAAG |
| SEQ ID | αhCCR2 | GAGGTGCAGCTGGTTGAATCTGGCGGAGGATTGGTTCAGCCTGGCGGCT |
| NO: 21 | 43G12 VH | CTCTGAGACTGTCTTGTGTGGCCTCTGGCTTCACCTTCTCCGACTACTGG |
| | | ATGTCCTGGGTCCGACAGGCTCCTGGCAAAGGACTGGAATGGGTCGCCA |
| | | ACATCAAGAAAGACGGCTCCGTGAACTACTACGTGGACTCCGTGAAGGG |
| | | CAGATTCACCATCTCTCGGGACAACGCCAAGAACTCCCTGTACCTGCAG |
| | | ATGAACAGCCTGAGAGCCGAGGACACCGCCGTGTACTACTGCACCAGAT |
| | | TCGATTACTGGGGCCAGGGCACCCTGGTCACAGTGTCCTCT |
| SEQ ID | αhCCR2 | CAGGCTGGCTTGACCCAGCCTCCTAGCGTTTCCAAGGGCCTGAGACAGA |
| NO: 22 | 43G12 VL | CCGCCACACTGACCTGTACCGGCAACTCTAACAACGTGGGCAATCAGGG |
| | | CGCTGCCTGGTTGCAGCAGCATCAGGGACATCCTCCAAAGCTGCTGTTCT |
| | | ACCGGAACAACAACAGAGCCTCCGGCATCTCCGAGCGGCTGTCTGCTTC |
| | | TAGATCCGGCAATACCGCCAGCCTGACCATCACTGGACTGCAGCCTGAG |
| | | GACGAGGCCGACTACTATTGCCTGACCTGGGACTCCTCTCTGTCCGTGGT |
| | | GGTTTTTGGCGGAGGCACCAAGCTGACAGTGCTG |
| SEQ ID | αhCSF1R | CAGGTGCAGCTGGTTCAGTCTGGCGCCGAAGTGAAGAAACCTGGCGCCT |
| NO: 23 | emactuzumab | CTGTGAAGGTGTCCTGCAAGGCTTCCGGCTACACCTTTACCAGCTACGAC |
| | VH | ATCTCCTGGGTCCGACAGGCTCCTGGACAAGGCTTGGAATGGATGGGCG |
| | | TGATCTGGACCGATGGCGGCACCAATTACGCCCAGAAACTGCAGGGCAG |
| | | AGTGACCATGACCACCGACACCTCTACCTCCACCGCCTACATGGAACTG |
| | | CGGTCCCTGAGATCTGACGACACCGCCGTGTACTACTGCGCCAGAGATC |
| | | AGCGGCTGTACTTCGATGTGTGGGGCCAGGGCACAACCGTGACAGTGTC |
| | | CTCT |
| SEQ ID | αhCSF1R | GACATCCAGATGACCCAGTCTCCATCCTCTCTGTCCGCCTCTGTGGGCGA |
| NO: 24 | emactuzumab | CAGAGTGACCATCACCTGTAGAGCCTCCGAGGACGTGAACACCTACGTG |
| | VL | TCCTGGTATCAGCAGAAGCCCGGCAAGGCTCCCAAGCTGCTGATCTACG |
| | | CCGCCTCTAACAGATACACCGGCGTGCCCTCTAGATTCTCCGGCTCTGGC |
| | | TCTGGCACCGACTTTACCCTGACAATCTCCAGCCTGCAGCCTGAGGACTT |
| | | CGCCACCTACTACTGCCAGCAGTCCTTCAGCTACCCCACCTTTGGCCAGG |
| | | GCACCAAGCTGGAAATCAAG |
| SEQ ID | hFc Hole | GATAAGACCCACACCTGTCCTCCCTGCCCTGCCCCTGAACTGCTGGGCGG |
| NO: 25 | | ACCTAGCGTGTTCCTGTTCCCTCCAAAGCCCAAGGACACCCTGATGATCA |
| | | GCCGGACCCCTGAAGTGACCTGCGTGGTGGTGGATGTGTCCCACGAGGA |
| | | TCCCGAAGTGAAGTTCAATTGGTACGTGGACGGCGTGGAAGTGCACAAC |
| | | GCCAAGACCAAGCCCAGAGAGGAACAGTACAACAGCACCTACCGGGTG |
| | | GTGTCCGTGCTGACCGTGCTGCACCAGGACTGGCTGAACGGCAAAGAGT |
| | | ACAAGTGCAAGGTGTCCAACAAGGCCCTGCCAGCCCCTATCGAGAAAAC |
| | | CATCAGCAAGGCCAAGGGCCAGCCTAGAGAGCCTCAGGTCTGCACCCTG |
| | | CCTCCCAGCCGGGAAGAGATGACCAAGAACCAGGTGTCCCTGAGCTGCG |
| | | CCGTGAAGGGCTTCTACCCCTCCGATATCGCCGTGGAATGGGAGAGCAA |
| | | CGGCCAGCCCGAGAACAACTACAAGACCACCCCTCCCGTGCTGGACAGC |
| | | GACGGCAGCTTCTTCCTGGTGTCCAAACTGACCGTGGACAAGAGCCGGT |
| | | GGCAGCAGGGCAATGTGTTCAGCTGTAGCGTGATGCACGAGGCCCTGCA |
| | | CAACCACTACACCCAGAAGTCTCTGAGCCTGAGCCCTGGCAAA |
| SEQ ID | αhCSF1R | CAGGTGCAGCTGGTTCAGTCTGGCGCCGAAGTGAAGAAACCTGGCTCCT |
| NO: 26 | cabiralizumab | CCGTGAAGGTGTCCTGCAAGGCTTCTGGCTACACCTTTACCGACAACTAC |
| | VH | ATGATCTGGGTCCGACAGGCTCCTGGACAGGGACTTGAGTGGATGGGCG |
| | | ACATCAACCCTTACAACGGCGGCACCACCTTCAACCAGAAATTCAAGGG |
| | | CAGAGTGACCATCACCGCCGACAAGTCTACCTCCACCGCCTACATGGAA |
| | | CTGTCCAGCCTGAGATCTGAGGACACCGCCGTGTACTACTGCGCCAGAG |
| | | AGTCCCCTTACTTCTCCAACCTGTACGTGATGGACTACTGGGGCCAGGGC |
| | | ACACTGGTCACAGTGTCCTCT |
| SEQ ID | αhCSF1R | GAGATCGTGCTGACCCAGTCTCCTGCCACACTGTCACTGTCTCCAGGCGA |
| NO: 27 | cabiralizumab | GAGAGCTACCCTGTCCTGCAAGGCTTCTCAGTCCGTGGACTACGACGGC |
| | VL | GACAACTACATGAACTGGTATCAGCAGAAGCCCGGCCAGGCTCCTAGAC |
| | | TGCTGATCTACGCCGCCTCCAACCTGGAATCTGGCATCCCCGCTAGATTC |
| | | TCCGGCTCTGGCTCTGGCACAGACTTTACCCTGACCATCTCCAGCCTGGA |
| | | ACCTGAGGACTTCGCCGTGTACTACTGCCACCTGTCCAACGAGGACCTGT |
| | | CCACATTTGGCGGAGGCACCAAGGTGGAAATCAAG |

TABLE 2
Sequences used to construct ORFs.

| Full length | Variable | Constant | Fc |
|---|---|---|---|
| SEQ ID NO: 28 | SEQ ID NO: 1 | SEQ ID NO: 3 | SEQ ID NO: 9 |
| SEQ ID NO: 29 | SEQ ID NO: 2 | SEQ ID NO: 4 | |
| SEQ ID NO: 30 | SEQ ID NO: 5 | SEQ ID NO: 6 | SEQ ID NO: 10 |
| SEQ ID NO: 31 | SEQ ID NO: 7 | SEQ ID NO: 8 | |
| SEQ ID NO: 32 | SEQ ID NO: 11 | SEQ ID NO: 12 | SEQ ID NO: 13 |
| SEQ ID NO: 33 | SEQ ID NO: 14 | SEQ ID NO: 15 | |
| SEQ ID NO: 34 | SEQ ID NO: 16 | SEQ ID NO: 12 | SEQ ID NO: 13 |
| SEQ ID NO: 35 | SEQ ID NO: 17 | SEQ ID NO: 18 | |
| SEQ ID NO: 36 | SEQ ID NO: 19 | SEQ ID NO: 12 | SEQ ID NO: 13 |
| SEQ ID NO: 37 | SEQ ID NO: 20 | SEQ ID NO: 15 | |
| SEQ ID NO: 38 | SEQ ID NO: 21 | SEQ ID NO: 12 | SEQ ID NO: 13 |
| SEQ ID NO: 39 | SEQ ID NO: 22 | SEQ ID NO: 18 | |
| SEQ ID NO: 40 | SEQ ID NO: 23 | SEQ ID NO: 12 | SEQ ID NO: 25 |
| SEQ ID NO: 41 | SEQ ID NO: 24 | SEQ ID NO: 15 | |
| SEQ ID NO: 42 | SEQ ID NO: 26 | SEQ ID NO: 12 | SEQ ID NO: 25 |
| SEQ ID NO: 43 | SEQ ID NO: 27 | SEQ ID NO: 15 | |

TABLE 3
Nucleic acid sequences of ORFs.

| | Nucleic Acid Sequence |
|---|---|
| SEQ ID | ATGGAAACCGACACACTGCTGCTGTGGGTGCTGCTCTTGTGGGTGCCAGGATCTACAGG |
| NO: 28 | ACAGGTCCAGCTGCAAGAGTCTGGCCCTGGACTGGTTCAGCCCTCTCAGACCCTGTCTCT |
| | GACCTGTACCGTGTCCGGCTTCTCCCTGACCGACTTCTCTGTGCACTGGGTCCGACAGCC |
| | TCCAGGCAAAGGACTGGAATGGATGGGCAGAATCAGATCCGAGGGCAACACCGACTAC |
| | AACAGCGCCCTGAAGTCCCGGCTGTCTATCAGCAGAGACACCTCCAAGAGCCAGGTGTT |
| | CCTGAAGATGAACTCCCTGCAGACCGAGGACACCGCCATCTATTTCTGCACCAGAGGCG |
| | ACATCCTCGGCTTCGGCTATTGGGGACAGGGCGTGATGGTCACCGTTAGCTCTGCTCAG |
| | ACCACCGCTCCTAGCGTGTACCCTTTGGCTCCTGGCTGTGGCGACACCACCTCTTCTACA |
| | GTGACCCTGGGCTGTCTGGTCAAGGGCTACTTTCCTGAGCCTGTGACCGTGACCTGGAAC |
| | TCTGGTGCCCTGTCCTCCGACGTGCACACCTTTCCAGCTGTGCTGCAGTCCGGCCTGTAC |
| | ACCCTGACATCCTCCGTGACCTCTTCCACCTGGCCTAGCCAGACCGTGACATGCAATGTG |
| | GCTCACCCTGCCTCCAGCACCAAGGTGGACAAGAAGGTGGAACGGCGGACCATTAAGC |
| | CTTGTCCTCCATGCAAGTGCCCCGCTCCTAATCTGCTCGGAGGCCCTTCCGTGTTCATCTT |
| | TCCACCTAAGATCAAGGACGTGCTGATGATCTCCCTGTCTCCTATCGTGACCTGCGTGGT |
| | GGTGGACGTGTCCGAGGATGATCCTGACGTGCAGATCAGTTGGTTCGTGAACAACGTGG |
| | AAGTGCACACCGCTCAGACCCAGACACACAGAGAGGACTACAACTCTACCCTGAGAGT |
| | GGTGTCTGCCCTGCCTATCCAGCATCAGGACTGGATGTCCGGCAAAGAATTCAAGTGCA |
| | AAGTGAACAACAAGGACCTGCCTGCTCCAATCGAGCGGACCATCTCTAAGCCTAAGGGC |
| | TCTGTCAGGGCCCCTCAGGTGTACGTTCTGCCTCCTTGCGAGGAAGAGATGACCAAGAA |
| | ACAAGTGACACTGTGGTGCATGGTCACAGACTTCATGCCCGAGGACATCTACGTGGAAT |
| | GGACCAACAACGGCAAGACCGAGCTGAACTACAAGAACACCGAGCCTGTGCTGGACTC |
| | CGACGGCTCCTACTTCATGTACTCCAAGCTGCGCGTCGAGAAGAAGAACTGGGTCGAGA |
| | GAAACTCCTACTCCTGCTCCGTGGTGCACGAGGGCCTGCACAATCACCACACCACCAAG |
| | TCCTTCTCTCGGACCCCTGGCAAGTGATGA |
| SEQ ID | ATGGAAACCGACACACTGCTGCTGTGGGTGCTGCTCTTGTGGGTGCCAGGATCTACCGG |
| NO: 29 | CGACATCGTGATGACCCAGTCTCCACTGTCCGTGTCTGTGACCCCTGGCGAGTCTGCCTC |
| | CATCTCCTGCAGATCCTCCAAGAGCCTGCTGCACTTCAAGGGCATCACCTTCGTGTACTG |
| | GTATCTGCAGAAGCCCGGCCAGTCTCCTCAGCTGCTGATCTTCAGAATGTCCAGCCTGGC |
| | CTCTGGCGTGCCCGATAGATTTTCTGGCTCCGGCTCCGAGACAGACTTCACCCTGAAGAT |
| | CTCCAGAGTGGAAGCCGAGGACGTGGGCACCTACTATTGTGGCCAGCTGCTGGAAAACC |
| | CCTACACCTTTGGCGCTGGCACCAAGCTGGAACTGAAGAGAGCTGACGCTGCCCCTACC |
| | GTGTCTATCTTCCCTCCATCCATGGAACAGCTGACCTCTGGCGGAGCTACCGTCGTGTGC |
| | TTCGTGAACAACTTCTACCCTCGGGACATCTCCGTGAAGTGGAAGATCGACGGCTCTGA |
| | GCAGCGAGATGGCGTGCTGGATTCTGTGACCGACCAGGACTCCAAGGACAGCACCTACT |
| | CCATGTCTAGCACCCTGAGCCTGACCAAGGTGGAATACGAGCGGCACAACCTGTATACC |
| | TGCGAGGTGGTGCACAAGACCTCCAGCTCTCCCGTGGTCAAGTCCTTCAACCGGAACGA |
| | GTGCTGATGA |
| SEQ ID | ATGGAAACCGACACACTGCTGCTGTGGGTGCTGCTCTTGTGGGTGCCAGGATCTACAGG |
| NO: 30 | ACAGGTCCAGTTGCAGCAGTCTGGCGCTGAGCTGGTCAAGCCTGGATCCTCCGTGAAGA |
| | TCTCCTGCAAGGCCTCCGGCTACACCTTCACCTCCAACTTCATGCACTGGATCAAGCAGC |
| | AGCCCGGCAACGGCCTGGAATGGATCGGATGGATCTATCCTGGCGACGGCGACACCGA |
| | GTACAACCAGAAGTTCAACGGCAAGGCTACCCTGACCGCCGACAAGTCCTCTTCCACCG |
| | CTTACATGCAGCTGTCCAGCCTGACCTCTGAGGACTCCGCCGTGTACTTCTGCGCCGTGA |
| | ATTATGGCGGCTACGTGCTGGATGCTTGGGGCCAAGGCGCTTCTGTGACAGTGTCCTCTG |
| | CCGAGACAACCGCTCCTAGCGTTTACCCTCTGGCTCCTGGCACAGCCCTGAAGTCCAACT |
| | CTATGGTCACCCTGGGCTGCCTGGTCAAGGGCTACTTTCCTGAGCCTGTGACCGTGACCT |
| | GGAACTCTGGTGCTCTGTCTAGCGGCGTGCACACCTTTCCAGCTGTGCTGCAGAGCGGCC |
| | TGTACACCCTGACATCTAGCGTGACCGTGCCTTCCAGCACCTGGTCTAGTCAGGCTGTGA |
| | CCTGCAACGTGGCCCATCCTGCCTCTTCTACCAAGGTGGACAAGAAAATCGTGCCCAGA |
| | GAGTGCAACACCATCAAGCCCTGTCCTCCATGCAAGTGCCCCGCTCCTAATCTGCTCGGA |
| | GGCCCTTCCGTGTTCATCTTCCCACCTAAGATCAAGGACGTGCTGATGATCTCCCTGTCT |
| | CCTATCGTGACCTGCGTGGTGGTGGACGTGTCCGAGGATGATCCTGACGTGCAGATCAG |
| | TTGGTTCGTGAACAACGTGGAAGTGCACACCGCTCAGACCCAGACACACAGAGAGGACT |
| | ACAACAGCACCCTGAGAGTGGTGTCTGCCCTGCCAATCCAGCACCAGGATTGGATGTCC |
| | GGCAAAGAATTCAAGTGCAAAGTGAACAACAAGGACCTGCCTGCTCCAATCGAGCGGA |
| | CCATCTCTAAGCCTAAGGGCTCTGTGCGGGCTCCCCAAGTTTGTGTTCTGCCTCCACCTG |
| | AGGAAGAGATGACCAAGAAACAAGTGACCCTGTCTTGTGCCGTGACCGACTTCATGCCC |
| | GAGGACATCTACGTGGAATGGACCAACAATGGCAAGACCGAGCTGAACTACAAGAACA |
| | CCGAGCCTGTGCTGGACTCCGACGGCTCCTACTTCATGGTGTCTAAGCTGCGCGTCGAGA |
| | AGAAGAACTGGGTCGAGAGAAACTCCTACTCCTGCTCCGTGGTGCACGAGGGCCTGCAC |
| | AATCACCACACCACCAAGTCCTTCTCTCGGACCCCTGGCAAGTGATGA |
| SEQ ID | ATGGAAACCGACACACTGCTGCTGTGGGTGCTGCTCTTGTGGGTGCCAGGATCTACAGG |
| NO: 31 | CGAGATCGTGCTGACCCAGTCTCCTACCACCATGGCTGCTAGCCCTGGCGAGAAAGTGA |
| | CAATTACCTGCCGGGCCTCCTCCTCCACCAACTACATGTCCTGGTATCAGCAGAAGTCCG |
| | GCGCCTCTCCTAAGCCTTGGATCTACGAGACATCCAAGCTGGCCTCTGGCGTGCCCGATA |
| | GATTTTCCGGCTCTGGCTCCGGCACCTCCTACAGCTTCACCATCTCCAGCATGGAAACAG |
| | AGGACGCCGCCACCTACTACTGCCACCAGTGGTCATCTACCCCTCTGACCTTTGGCAGCG |
| | GCACCAAGCTGGAAATCAAGAGAGCTGACGCCGCTCCTACCGTGTCTATCTTCCCTCCAT |
| | CCATGGAACAGCTGACCTCCGGCGGAGCTACCGTCGTGTGTTTCGTGAACAACTTCTACC |
| | CTCGGGACATCTCCGTGAAGTGGAAGATCGACGGCTCTGAGCAGCGAGATGGCGTGCTG |
| | GATTCTGTGACCGACCAGGACTCCAAGGACAGCACCTACTCCATGTCTAGCACCCTGAG |
| | CCTGACCAAGGTGGAATACGAGCGGCACAACCTGTATACCTGCGAGGTGGTGCACAAG |
| | ACCTCCAGCTCTCCCGTGGTCAAGTCCTTCAACCGGAACGAGTGCTGATGA |
| SEQ ID | ATGGAAACCGACACACTGCTGCTGTGGGTGCTGCTCTTGTGGGTGCCAGGATCTACAGG |
| NO: 32 | CGAGGTGCAGCTGGTTGAATCTGGCGGAGGACTGGTTAAGCCTGGCGGCTCTCTGAGAC |
| | TGTCTTGTGCCGCTTCTGGCTTCACCTTCTCCGCCTACGCCATGAACTGGGTCCGACAGG |
| | CTCCTGGCAAAGGCCTGGAATGGGTCGGAAGAATCCGGACCAAGAACAACAACTACGC |
| | CACCTACTACGCCGACTCCGTGAAGGACCGGTTCACCATCTCTCGGGACGACTCCAAGA |
| | ACACCCTGTACCTGCAGATGAACTCCCTGAAAACCGAGGACACCGCCGTGTACTACTGC |
| | ACCACCTTCTACGGCAATGGCGTGTGGGGACAGGGCACACTGGTTACCGTTTCTTCCGCC |
| | TCCACCAAGGGACCCTCTGTGTTTCCTCTGGCTCCCTCCAGCAAGTCTACCTCTGGTGGA |
| | ACAGCTGCCCTGGGCTGCCTGGTCAAGGATTACTTTCCTGAGCCTGTGACCGTGTCCTGG |
| | AACTCTGGCGCTCTGACATCTGGCGTGCACACCTTTCCAGCTGTGCTGCAGTCCTCTGGC |
| | CTGTACTCTCTGTCCTCCGTCGTGACCGTGCCTTCTAGCTCTCTGGGCACCCAGACCTAC |
| | ATCTGCAATGTGAACCACAAGCCTTCCAACACCAAGGTGGACAAGAGAGTGGAACCCA |
| | AGTCCTGCGACAAGACCCACACCTGTCCTCCATGTCCTGCTCCAGAACTGCTCGGCGGA |
| | CCTTCCGTGTTCCTGTTTCCTCCAAAGCCTAAGGACACCCTGATGATCTCTCGGACCCCT |
| | GAAGTGACCTGCGTGGTGGTGGATGTGTCTCACGAGGATCCCGAAGTGAAGTTCAATTG |
| | GTACGTGGACGGCGTGGAAGTGCACAACGCCAAGACCAAGCCTAGAGAGGAACAGTAC |
| | AACTCCACCTACAGAGTGGTGTCCGTGCTGACCGTGCTGCACCAGGATTGGCTGAACGG |
| | CAAAGAGTACAAGTGCAAGGTGTCCAACAAGGCCCTGCCTGCTCCTATCGAAAAGACCA |
| | TCTCCAAGGCCAAGGGCCAGCCTAGGGAACCCCAGGTTTACACCCTGCCTCCATGCCGG |
| | GAAGAGATGACCAAGAATCAGGTGTCCCTGTGGTGCCTCGTGAAGGGCTTCTACCCTTC |
| | CGATATCGCCGTGGAATGGGAGAGCAATGGCCAGCCTGAGAACAACTACAAGACAACC |
| | CCTCCTGTGCTGGACTCCGACGGCTCATTCTTCCTGTACTCCAAGCTGACAGTGGACAAG |
| | TCCAGATGGCAGCAGGGCAACGTGTTCTCCTGCTCCGTGATGCACGAGGCCCTGCACAA |
| | TCACTACACCCAGAAGTCCCTGTCTCTGTCCCCTGGCAAGTGATGA |
| SEQ ID | ATGGAAACCGACACACTGCTGCTGTGGGTGCTGCTCTTGTGGGTGCCAGGATCTACAGG |
| NO: 33 | CGACGTGGTCATGACACAGAGCCCTCTGTCTCTGCCCGTGACATTGGGACAGCCTGCCTC |
| | CATCTCCTGCAAGTCCTCTCAGTCCCTGCTGGACTCTGACGGCAAGACCTTCCTGAACTG |
| | GTTCCAGCAGCGGCCTGGCCAGTCTCCTAGAAGGCTGATCTACCTGGTGTCCAAGCTGG |
| | ATTCTGGCGTGCCCGACAGATTCTCCGGCTCTGGCTCTGGCACCGACTTCACCCTGAAGA |
| | TCTCCAGAGTGGAAGCCGAGGACGTGGGCGTGTACTACTGTTGGCAGGGCACCCACTTT |
| | CCATACACCTTCGGCCAGGGCACCAGACTGGAAATCAAGAGAACCGTGGCCGCTCCTTC |
| | CGTGTTCATCTTCCCACCTTCCGACGAGCAGCTGAAGTCCGGCACAGCTTCTGTCGTGTG |
| | CCTGCTGAACAACTTCTACCCTCGGGAAGCCAAGGTGCAGTGGAAGGTGGACAATGCCC |
| | TGCAGTCCGGCAACTCCCAAGAGTCTGTGACCGAGCAGGACTCCAAGGACAGCACCTAC |
| | AGCCTGTCCAGCACACTGACCCTGTCCAAGGCCGACTACGAGAAGCACAAGGTGTACGC |
| | CTGCGAAGTGACCCATCAGGGCCTGTCTAGCCCTGTGACCAAGTCTTTCAACCGGGGCG |
| | AGTGCTGATGA |
| SEQ ID | ATGGAAACCGACACACTGCTGCTGTGGGTGCTGCTCTTGTGGGTGCCAGGATCTACAGG |
| NO: 34 | CGAAGTGCAGCTGGTTCAGTCTGGCGCCGAAGTGAAGAAACCTGGCGCCTCTGTGAAGG |
| | TGTCCTGCAAGGCTTCTGGCTACACCTTTACCGGCTACCACATGCACTGGGTCCGACAGG |
| | CTCCAGGACAAGGCTTGGAATGGATGGGCTGGATCAACCCCAACTCCGGCGTGACCAAA |
| | TACGCCCAGAAATTCCAGGGCAGAGTGACCATGACCAGAGACACCTCCATCAACACCGC |
| | CTACATGGAACTGTCCCGGCTGAGATTCGACGACACCGACGTGTACTACTGTGCCACCG |
| | GCGGCTTTGGCTATTGGGGAGAGGGAACACTGGTCACCGTGTCCTCCGCTTCTACCAAG |
| | GGACCCTCCGTGTTTCCTCTGGCTCCTTCCAGCAAGTCTACCTCCGGTGGAACAGCTGCT |
| | CTGGGCTGCCTGGTCAAGGACTACTTTCCTGAGCCTGTGACCGTGTCTTGGAACTCTGGC |
| | GCTCTGACATCCGGCGTGCACACCTTTCCAGCTGTGCTGCAATCCTCCGGCCTGTACTCT |
| | CTGTCCTCCGTCGTGACCGTGCCTTCTAGCTCTCTGGGCACCCAGACCTACATCTGCAAT |
| | GTGAACCACAAGCCTTCCAACACCAAGGTGGACAAGAGAGTGGAACCCAAGTCCTGCG |
| | ACAAGACCCACACCTGTCCTCCATGTCCTGCTCCAGAACTGCTCGGCGGACCTTCTGTGT |
| | TCCTGTTTCCTCCAAAGCCTAAGGACACCCTGATGATCTCTCGGACCCCTGAAGTGACCT |
| | GCGTGGTGGTGGATGTGTCTCACGAGGACCCAGAAGTGAAGTTCAATTGGTACGTGGAC |
| | GGCGTGGAAGTGCACAACGCCAAGACCAAGCCTAGAGAGGAACAGTACAACTCCACCT |
| | ACAGAGTGGTGTCCGTGCTGACCGTGCTGCACCAGGATTGGCTGAACGGCAAAGAGTAC |
| | AAGTGCAAGGTGTCCAACAAGGCCCTGCCTGCTCCTATCGAAAAGACCATCTCCAAGGC |
| | CAAGGGCCAGCCTAGGGAACCCCAGGTTTACACCCTGCCTCCATGCCGGGAAGAGATGA |
| | CCAAGAACCAGGTGTCCCTGTGGTGCCTCGTGAAGGGCTTCTACCCTTCCGATATCGCCG |
| | TGGAATGGGAGAGCAATGGCCAGCCTGAGAACAACTACAAGACAACCCCTCCTGTGCTG |
| | GACTCCGACGGCTCATTCTTCCTGTACTCCAAGCTGACAGTGGACAAGTCCAGATGGCA |
| | GCAGGGCAACGTGTTCTCCTGCTCCGTGATGCACGAGGCCCTGCACAATCACTACACAC |
| | AGAAGTCCCTGTCTCTGTCCCCTGGCAAGTGATGA |
| SEQ ID | ATGGAAACCGACACACTGCTGCTGTGGGTGCTGCTCTTGTGGGTGCCAGGATCTACAGG |
| NO: 35 | ACTGCCCGTGTTGACCCAGCCTCCTAGCGTTTCCAAGGGCCTGAGACAGACCGCCACAC |
| | TGACCTGTACCGGCAACTCTAACAACGTGGGCAATCAGGGCGCTGCCTGGTTGCAGCAG |
| | CATCAGGGACAGCCTCCAAAGCTGCTGTCCTACCGGAACCACAACAGACCTAGCGGCGT |
| | GTCCGAGCGGTTCAGCCCTTCTAGATCTGGCGACACCTCCAGCCTGACCATCACTGGACT |
| | GCAGCCTGAGGACGAGGCCGACTACTATTGTCTGGCCTGGGACAGCTCCCTGCGGGCCT |
| | TTGTTTTTGGCACCGGCACCAAGCTGACCGTGCTGGGACAACCTAAGGCCAATCCTACC |
| | GTGACACTGTTCCCTCCATCCTCCGAGGAACTGCAGGCCAACAAGGCTACCCTCGTGTG |
| | CCTGATCTCCGACTTTTACCCTGGCGCTGTGACCGTGGCCTGGAAGGCTGATGGATCTCC |
| | TGTGAAGGCTGGCGTGGAAACCACCAAGCCTTCCAAGCAGTCCAACAACAAATACGCCG |
| | CCTCCTCCTACCTGTCTCTGACCCCTGAACAGTGGAAGTCCCACCGGTCCTACAGCTGCC |
| | AAGTGACCCATGAGGGCTCCACCGTGGAAAAGACCGTGGCTCCTACCGAGTGCTCCTGA |
| | TGA |
| SEQ ID | ATGGAAACCGACACACTGCTGCTGTGGGTGCTGCTCTTGTGGGTGCCAGGATCTACAGG |
| NO: 36 | ACAGGTGCAGCTGGTTCAGTCTGGCGCCGAAGTGAAGAAACCTGGCGCCTCTGTGAAGG |
| | TGTCCTGCAAGGCTTCCGGCTACACCTTCTCCAGCTACTACATGCACTGGGTCCGACAGG |
| | CCCCTGGACAAGGATTGGAGTGGATGGGCATCATCAACCCCTCTGGCGGCAACACCTCT |
| | TACGCCCAGAAATTCCAGGGCAGAGTGACCATGACCAGAGACACCTCCACCAGCACCGT |
| | GTACATGGAACTGTCCAGCCTGAGATCCGAGGACACCGCCGTGTACTACTGTGCCAGAG |
| | GCGGATACCAGCTGCCTCACGGTAGAGCCAGAGCCTTCGATATGTGGGGCCAGGGCACA |
| | ATGGTCACCGTGTCCTCTGCTTCCACCAAGGGACCCTCTGTGTTCCCTCTGGCTCCTTCCA |
| | GCAAGTCCACATCCGGTGGAACAGCTGCTCTGGGCTGCCTGGTCAAGGACTACTTTCCT |
| | GAGCCTGTGACCGTGTCTTGGAACTCTGGCGCTCTGACATCCGGCGTGCACACATTTCCA |
| | GCTGTGCTGCAGTCCTCCGGCCTGTACTCTCTGTCCTCTGTCGTGACCGTGCCTTCCAGCT |
| | CTCTGGGAACCCAGACCTACATCTGCAATGTGAACCACAAGCCTTCCAACACCAAGGTG |
| | GACAAGAGAGTGGAACCCAAGTCCTGCGACAAGACCCACACCTGTCCACCATGTCCTGC |
| | TCCAGAACTGCTCGGCGGACCTTCCGTGTTCCTGTTTCCTCCAAAGCCTAAGGACACCCT |
| | GATGATCTCTCGGACCCCTGAAGTGACCTGCGTGGTGGTGGATGTGTCCCACGAGGACC |
| | CAGAAGTGAAGTTCAATTGGTACGTGGACGGCGTGGAAGTGCACAACGCCAAGACCAA |
| | GCCTAGAGAGGAACAGTACAACTCCACCTACAGAGTGGTGTCCGTGCTGACCGTGCTGC |
| | ACCAGGATTGGCTGAACGGCAAAGAGTACAAGTGCAAGGTGTCCAACAAGGCCCTGCC |
| | TGCTCCTATCGAAAAGACCATCTCCAAGGCCAAGGGCCAGCCTAGGGAACCCCAGGTTT |
| | ACACCCTGCCTCCATGCCGGGAAGAGATGACCAAGAACCAGGTGTCCCTGTGGTGCCTC |
| | GTGAAGGGCTTCTACCCTTCCGATATCGCCGTGGAATGGGAGAGCAATGGCCAGCCAGA |
| | GAACAACTACAAGACAACCCCTCCTGTGCTGGACTCCGACGGCTCATTCTTCCTGTACTC |
| | CAAGCTGACAGTGGACAAGTCCAGATGGCAGCAGGGCAACGTGTTCTCCTGCTCCGTGA |
| | TGCACGAGGCCCTGCACAATCACTACACACAGAAGTCCCTGTCTCTGTCCCCTGGCAAG |
| | TGATGA |
| SEQ ID | ATGGAAACCGACACACTGCTGCTGTGGGTGCTGCTCTTGTGGGTGCCAGGATCTACCGG |
| NO: 37 | CGCCATCAGAATGACCCAGTCTCCACTGAGCCTGCCTGTGACATTGGGCCAGCCTGCCTC |
| | TATCTCCTGCACCTCCTCTCAGTCTCTGGTGTACAGAGATGGCACCACCTACCTGAACTG |
| | GTTCCAGCAGAGGCCTGGCCAGTCTCCTAGACGGCTGATCTACAAGGTGTCCAACAGAG |
| | ACTCTGGCGTGCCCGACAGATTCACCGGCTCTGGCTCTGGCACCACATTCACCCTGACCA |
| | TCTCCAGAGTGGAAGCCGAGGACGTGGGCATCTACTACTGTATGCAGGGCACCCACTGG |
| | CCTCTGACCTTTGGCCAGGGAACAAAGGTGGAAATCAAGCGGACCGTGGCCGCTCCTTC |
| | CGTGTTCATCTTCCCACCTTCCGACGAGCAGCTGAAGTCTGGCACAGCCTCTGTCGTGTG |
| | CCTGCTGAACAACTTCTACCCTCGGGAAGCCAAGGTGCAGTGGAAGGTGGACAATGCCC |
| | TGCAGTCCGGCAACTCCCAAGAGTCTGTGACCGAGCAGGACTCCAAGGACAGCACCTAC |
| | AGCCTGTCCTCCACACTGACCCTGTCCAAGGCCGACTACGAGAAGCACAAGGTGTACGC |
| | CTGCGAAGTGACCCATCAGGGCCTGTCTAGCCCTGTGACCAAGTCTTTCAACCGGGGCG |
| | AGTGCTGATGA |
| SEQ ID | ATGGAAACCGACACACTGCTGCTGTGGGTGCTGCTCTTGTGGGTGCCAGGATCTACAGG |
| NO: 38 | CGAGGTGCAGCTGGTTGAATCTGGCGGAGGATTGGTTCAGCCTGGCGGCTCTCTGAGAC |
| | TGTCTTGTGTGGCCTCTGGCTTCACCTTCTCCGACTACTGGATGTCCTGGGTCCGACAGG |
| | CTCCTGGCAAAGGACTGGAATGGGTCGCCAACATCAAGAAAGACGGCTCCGTGAACTAC |
| | TACGTGGACTCCGTGAAGGGCAGATTCACCATCTCTCGGGACAACGCCAAGAACTCCCT |
| | GTACCTGCAGATGAACAGCCTGAGAGCCGAGGACACCGCCGTGTACTACTGCACCAGAT |
| | TCGATTACTGGGGCCAGGGCACCCTGGTCACAGTGTCCTCTGCTTCTACCAAGGGACCC |
| | AGCGTGTTCCCTCTGGCTCCTTCCAGCAAGTCTACCTCTGGCGGAACAGCTGCTCTGGGC |
| | TGCCTGGTCAAGGACTACTTTCCTGAGCCTGTGACCGTGTCCTGGAACTCTGGCGCTCTG |
| | ACATCTGGCGTGCACACCTTTCCAGCTGTGCTGCAGTCCTCCGGCCTGTACTCTCTGTCC |
| | TCTGTCGTGACCGTGCCTTCCAGCTCTCTGGGAACCCAGACCTACATCTGCAATGTGAAC |
| | CACAAGCCTTCCAACACCAAGGTGGACAAGAGAGTGGAACCCAAGTCCTGCGACAAGA |
| | CCCACACCTGTCCTCCATGTCCTGCTCCAGAACTGCTCGGCGGACCTTCCGTGTTCCTGT |
| | TTCCTCCAAAGCCTAAGGACACCCTGATGATCTCTCGGACCCCTGAAGTGACCTGCGTG |
| | GTGGTGGATGTGTCTCACGAGGATCCCGAAGTGAAGTTCAATTGGTACGTGGACGGCGT |
| | GGAAGTGCACAATGCCAAGACCAAGCCTAGAGAGGAACAGTACAACTCCACCTACAGA |
| | GTGGTGTCCGTGCTGACCGTGCTGCACCAGGATTGGCTGAACGGCAAAGAGTACAAGTG |
| | CAAGGTGTCCAACAAGGCCCTGCCTGCTCCTATCGAAAAGACCATCTCCAAGGCCAAGG |
| | GCCAGCCTAGGGAACCCCAGGTTTACACCCTGCCTCCATGCCGGGAAGAGATGACCAAG |
| | AACCAGGTGTCCCTGTGGTGCCTGGTTAAGGGCTTCTACCCCTCCGATATCGCCGTGGAA |
| | TGGGAGTCTAATGGCCAGCCAGAGAACAACTACAAGACAACCCCTCCTGTGCTGGACTC |
| | CGACGGCTCATTCTTCCTGTACTCCAAGCTGACAGTGGACAAGTCCAGATGGCAGCAGG |
| | GCAACGTGTTCTCCTGCTCCGTGATGCACGAGGCCCTGCACAATCACTACACCCAGAAG |
| | TCCCTGTCTCTGTCCCCTGGCAAGTGATGA |
| SEQ ID | ATGGAAACCGACACACTGCTGCTGTGGGTGCTGCTCTTGTGGGTGCCAGGATCTACAGG |
| NO: 39 | ACAGGCTGGCTTGACCCAGCCTCCTAGCGTTTCCAAGGGCCTGAGACAGACCGCCACAC |
| | TGACCTGTACCGGCAACTCTAACAACGTGGGCAATCAGGGCGCTGCCTGGTTGCAGCAG |
| | CATCAGGGACATCCTCCAAAGCTGCTGTTCTACCGGAACAACAACAGAGCCTCCGGCAT |
| | CTCCGAGCGGCTGTCTGCTTCTAGATCCGGCAATACCGCCAGCCTGACCATCACTGGACT |
| | GCAGCCTGAGGACGAGGCCGACTACTATTGCCTGACCTGGGACTCCTCTCTGTCCGTGGT |
| | GGTTTTTGGCGGAGGCACCAAGCTGACAGTGCTGGGACAGCCTAAGGCCAATCCTACCG |
| | TGACACTGTTCCCTCCATCCTCCGAGGAACTGCAGGCCAACAAGGCTACCCTCGTGTGCC |
| | TGATCTCCGACTTTTACCCTGGCGCTGTGACCGTGGCCTGGAAGGCTGATGGATCTCCTG |
| | TGAAGGCTGGCGTGGAAACCACCAAGCCTTCCAAGCAGTCCAACAACAAATACGCCGCC |
| | TCCTCCTACCTGTCTCTGACCCCTGAACAGTGGAAGTCCCACCGGTCCTACAGCTGCCAA |
| | GTGACCCATGAGGGCTCCACCGTGGAAAAGACCGTGGCTCCTACCGAGTGCTCCTGATG |
| | A |
| SEQ ID | ATGGAAACCGACACACTGCTGCTGTGGGTGCTGCTCTTGTGGGTGCCAGGATCTACAGG |
| NO: 40 | ACAGGTGCAGCTGGTTCAGTCTGGCGCCGAAGTGAAGAAACCTGGCGCCTCTGTGAAGG |
| | TGTCCTGCAAGGCTTCCGGCTACACCTTTACCAGCTACGACATCTCCTGGGTCCGACAGG |
| | CTCCTGGACAAGGCTTGGAATGGATGGGCGTGATCTGGACCGATGGCGGCACCAATTAC |
| | GCCCAGAAACTGCAGGGCAGAGTGACCATGACCACCGACACCTCTACCTCCACCGCCTA |
| | CATGGAACTGCGGTCCCTGAGATCTGACGACACCGCCGTGTACTACTGCGCCAGAGATC |
| | AGCGGCTGTACTTCGATGTGTGGGGCCAGGGCACAACCGTGACAGTGTCCTCTGCTTCC |
| | ACCAAGGGACCCAGCGTTTTCCCTCTGGCTCCATCCTCCAAGTCTACCTCTGGCGGAACA |
| | GCTGCTCTGGGCTGCCTGGTCAAGGACTACTTTCCTGAGCCTGTGACCGTGTCCTGGAAC |
| | TCTGGCGCTCTGACATCTGGCGTGCACACATTCCCTGCTGTGCTGCAGTCCTCCGGCCTG |
| | TACTCTCTGTCCTCTGTGGTTACCGTGCCTTCCTCTAGCCTGGGCACCCAGACCTACATCT |
| | GCAATGTGAACCACAAGCCTTCCAACACCAAGGTGGACAAGAGAGTGGAACCCAAGTC |
| | CTGCGACAAGACCCACACCTGTCCACCATGTCCTGCTCCAGAACTGCTCGGCGGACCTTC |
| | CGTGTTCCTGTTTCCTCCAAAGCCTAAGGACACCCTGATGATCTCTCGGACCCCTGAAGT |
| | GACCTGCGTGGTGGTGGATGTGTCTCACGAGGACCCAGAAGTGAAGTTCAATTGGTACG |
| | TGGACGGCGTGGAAGTGCACAACGCCAAGACCAAGCCTAGAGAGGAACAGTACAACTC |
| | CACCTACAGAGTGGTGTCCGTGCTGACCGTGCTGCACCAGGATTGGCTGAACGGCAAAG |
| | AGTACAAGTGCAAGGTGTCCAACAAGGCCCTGCCTGCTCCTATCGAAAAGACCATCTCC |
| | AAGGCCAAGGGCCAGCCTCGGGAACCTCAAGTCTGTACCCTGCCTCCTAGCCGGGAAGA |
| | GATGACCAAGAACCAGGTGTCCCTGAGCTGCGCCGTGAAGGGCTTCTACCCTTCTGATA |
| | TCGCCGTGGAATGGGAGAGCAACGGCCAGCCTGAGAACAACTACAAGACAACCCCTCC |
| | TGTGCTGGACTCCGACGGCTCATTCTTCCTGGTGTCCAAGCTGACAGTGGACAAGTCCAG |
| | ATGGCAGCAGGGCAACGTGTTCTCCTGCTCCGTGATGCACGAGGCCCTGCACAATCACT |
| | ACACACAGAAGTCCCTGTCTCTGTCCCCTGGCAAGTGATGA |
| SEQ ID | ATGGAAACCGACACACTGCTGCTGTGGGTGCTGCTCTTGTGGGTGCCAGGATCTACCGG |
| NO: 41 | CGACATCCAGATGACCCAGTCTCCATCCTCTCTGTCCGCCTCTGTGGGCGACAGAGTGAC |
| | CATCACCTGTAGAGCCTCCGAGGACGTGAACACCTACGTGTCCTGGTATCAGCAGAAGC |
| | CCGGCAAGGCTCCCAAGCTGCTGATCTACGCCGCCTCTAACAGATACACCGGCGTGCCC |
| | TCTAGATTCTCCGGCTCTGGCTCTGGCACCGACTTTACCCTGACAATCTCCAGCCTGCAG |
| | CCTGAGGACTTCGCCACCTACTACTGCCAGCAGTCCTTCAGCTACCCCACCTTTGGCCAG |
| | GGCACCAAGCTGGAAATCAAGCGGACAGTGGCCGCTCCTTCCGTGTTCATCTTCCCACCT |
| | TCCGACGAGCAGCTGAAGTCCGGCACAGCTTCTGTCGTGTGCCTGCTGAACAACTTCTAC |
| | CCTCGGGAAGCCAAGGTGCAGTGGAAGGTGGACAATGCCCTGCAGTCCGGCAACTCCCA |
| | AGAGTCTGTGACCGAGCAGGACTCCAAGGACAGCACCTACAGCCTGTCCTCCACACTGA |
| | CCCTGTCCAAGGCCGACTACGAGAAGCACAAGGTGTACGCCTGCGAAGTGACCCATCAG |
| | GGCCTGTCTAGCCCTGTGACCAAGTCTTTCAACCGGGGCGAGTGCTGATGA |
| SEQ ID | ATGGAAACCGACACACTGCTGCTGTGGGTGCTGCTCTTGTGGGTGCCAGGATCTACAGG |
| NO: 42 | ACAGGTGCAGCTGGTTCAGTCTGGCGCCGAAGTGAAGAAACCTGGCTCCTCCGTGAAGG |
| | TGTCCTGCAAGGCTTCTGGCTACACCTTTACCGACAACTACATGATCTGGGTCCGACAGG |
| | CTCCTGGACAGGGACTTGAGTGGATGGGCGACATCAACCCTTACAACGGCGGCACCACC |
| | TTCAACCAGAAATTCAAGGGCAGAGTGACCATCACCGCCGACAAGTCTACCTCCACCGC |
| | CTACATGGAACTGTCCAGCCTGAGATCTGAGGACACCGCCGTGTACTACTGCGCCAGAG |
| | AGTCCCCTTACTTCTCCAACCTGTACGTGATGGACTACTGGGGCCAGGGCACACTGGTCA |
| | CAGTGTCCTCTGCTTCCACCAAGGGACCCAGCGTTTTCCCTCTGGCTCCATCCTCCAAGT |
| | CCACCTCTGGTGGAACAGCTGCTCTGGGCTGCCTGGTCAAGGACTACTTTCCTGAGCCTG |
| | TGACCGTGTCCTGGAACTCTGGCGCTCTGACATCTGGCGTGCACACCTTTCCAGCTGTGC |
| | TGCAGTCCTCCGGCCTGTACTCTCTGTCCTCTGTCGTGACCGTGCCTTCCAGCTCTCTGGG |
| | AACCCAGACCTACATCTGCAATGTGAACCACAAGCCTTCCAACACCAAGGTCGACAAGA |
| | GAGTGGAACCCAAGTCCTGCGACAAGACCCACACCTGTCCACCTTGTCCTGCTCCAGAA |
| | CTGCTCGGCGGACCTTCCGTGTTCCTGTTTCCTCCAAAGCCTAAGGACACCCTGATGATC |
| | TCTCGGACCCCTGAAGTGACCTGCGTGGTGGTGGATGTGTCTCACGAGGACCCAGAAGT |
| | GAAGTTCAATTGGTACGTGGACGGCGTGGAAGTGCACAACGCCAAGACCAAGCCTAGA |
| | GAGGAACAGTACAACTCCACCTACAGAGTGGTGTCCGTGCTGACCGTGCTGCACCAGGA |
| | TTGGCTGAACGGCAAAGAGTACAAGTGCAAGGTGTCCAACAAGGCCCTGCCTGCTCCTA |
| | TCGAAAAGACCATCTCCAAGGCCAAGGGCCAGCCTCGGGAACCTCAAGTCTGTACCCTG |
| | CCTCCTAGCCGGGAAGAGATGACCAAGAACCAGGTGTCCCTGAGCTGCGCCGTGAAGG |
| | GCTTCTACCCTTCTGATATCGCCGTGGAATGGGAGAGCAACGGCCAGCCAGAGAACAAC |
| | TACAAGACAACCCCTCCTGTGCTGGACTCCGACGGCTCATTCTTCCTGGTGTCCAAGCTG |
| | ACAGTGGACAAGTCCAGATGGCAGCAGGGCAACGTGTTCTCCTGCTCCGTGATGCACGA |
| | GGCCCTGCACAATCACTACACACAGAAGTCTCTGTCTCTGAGCCCCGGCAAGTGATGA |
| SEQ ID | ATGGAAACCGACACACTGCTGCTGTGGGTGCTGCTCTTGTGGGTGCCAGGATCTACAGG |
| NO: 43 | CGAGATCGTGCTGACCCAGTCTCCTGCCACACTGTCACTGTCTCCAGGCGAGAGAGCTA |
| | CCCTGTCCTGCAAGGCTTCTCAGTCCGTGGACTACGACGGCGACAACTACATGAACTGG |
| | TATCAGCAGAAGCCCGGCCAGGCTCCTAGACTGCTGATCTACGCCGCCTCCAACCTGGA |
| | ATCTGGCATCCCCGCTAGATTCTCCGGCTCTGGCTCTGGCACAGACTTTACCCTGACCAT |
| | CTCCAGCCTGGAACCTGAGGACTTCGCCGTGTACTACTGCCACCTGTCCAACGAGGACC |
| | TGTCCACATTTGGCGGAGGCACCAAGGTGGAAATCAAGCGGACAGTGGCCGCTCCTTCC |
| | GTGTTCATCTTCCCACCTTCCGACGAGCAGCTGAAGTCTGGCACCGCTTCTGTCGTGTGC |
| | CTGCTGAACAACTTCTACCCTCGGGAAGCCAAGGTGCAGTGGAAGGTGGACAATGCCCT |
| | GCAGTCCGGCAACTCCCAAGAGTCTGTGACCGAGCAGGACTCCAAGGACAGCACCTACA |
| | GCCTGTCCTCCACACTGACCCTGTCCAAGGCCGACTACGAGAAGCACAAGGTGTACGCC |
| | TGCGAAGTGACCCATCAGGGCCTGTCTAGCCCTGTGACCAAGTCTTTCAACCGGGGCGA |
| | GTGCTGATGA |

### 2. Expression and Purification.

The plasmids were co-transfected into either Expi293 cells (Life Technologies A14527) or ExpiCHO cells (Life Technologies A29127). Transfections were performed using 1 mg of total DNA for a multispecific construct with a 1:1 knob to hole heavy chain ratio and 3:2 light chain to heavy chain ratio. When biotinylation was required, 250 μg of BirA was added per liter in addition to the multispecific construct DNA. Transfection in Expi293 cells was done using linear 25,000 Da polyethylenimine (PEI, Polysciences Inc 23966) in a 3:1 ratio with the total DNA. The DNA and PEI were each added to 50 mL of OptiMem (Life Technologies 31985088) medium and sterile filtered. The DNA and PEI were combined for 10 minutes and added to the Expi293 cells with a cell density of 1.8-2.8×10⁶ cells/mL and a viability of at least 95%. The ExpiCHO transfection was performed according to the manufacturer's instructions. Expi293 cells were grown in a humidified incubator at 37° C. with 8% CO₂ for 5-7 days after transfection and ExpiCHO cells were grown for 14 days at 32° C. with 5% CO₂. The cells were pelleted by centrifugation at 4500×g and the supernatant was filtered through a 0.2 μm membrane. Protein A resin (GE 17-1279-03) was added to the filtered supernatant and incubated for 1-3 hours at room temperature. The resin was packed into a column, washed with 3×10 column volumes of Dulbecco's phosphate-buffered saline (DPBS, Life Technologies 14190-144). The bound protein was eluted from the column with 20 mM citrate, 100 mM NaCl, pH 2.9. When necessary, the proteins were further purified using ligand affinity and/or size exclusion chromatography on a Superdex 200 column with a running buffer of DPBS.

TABLE 4
Amino Acid Sequences.

| SEQ ID NO | Description | Amino Acid Sequence |
|---|---|---|
| SEQ ID NO: 44 | αCCR2 MC12 | QVQLQESGPGLVQPSQTLSLTCTVSGFSLTDFSVHWVRQPPGKGL |
| | VH | EWMGRIRSEGNTDYNSALKSRLSISRDTSKSQVFLKMNSLQTEDT |
| | | AIYFCTRGDILGFGYWGQGVMVTVSS |
| SEQ ID NO: 45 | αCCR2 MC12 | DIVMTQSPLSVSVTPGESASISCRSSKSLLHFKGITFVYWYLQKPGQ |
| | VL | SPQLLIFRMSSLASGVPDRFSGSGSETDFTLKISRVEAEDVGTYYCG |
| | | QLLENPYTFGAGTKLELK |
| SEQ ID NO: 46 | R2b CH1 | AQTTAPSVYPLAPGCGDTTSSTVTLGCLVKGYFPEPVTVTWNSGA |
| | | LSSDVHTFPAVLQSGLYTLTSSVTSSTWPSQTVTCNVAHPASSTKV |
| | | DKKVERR |
| SEQ ID NO: 47 | R2b CL | RADAAPTVSIFPPSMEQLTSGGATVVCFVNNFYPRDISVKWKIDGS |
| | | EQRDGVLDSVTDQDSKDSTYSMSSTLSLTKVEYERHNLYTCEVVH |
| | | KTSSSPVVKSFNRNEC |
| SEQ ID NO: 48 | αmCSF1R VH | QVQLQQSGAELVKPGSSVKISCKASGYTFTSNFMHWIKQQPGNGL |
| | | EWIGWIYPGDGDTEYNQKFNGKATLTADKSSSTAYMQLSSLTSED |
| | | SAVYFCAVNYGGYVLDAWGQGASVTVSS |
| SEQ ID NO: 49 | R2a CH1 | AETTAPSVYPLAPGTALKSNSMVTLGCLVKGYFPEPVTVTWNSGA |
| | | LSSGVHTFPAVLQSGLYTLTSSVTVPSSTWSSQAVTCNVAHPASST |
| | | KVDKKIVPRECN |
| SEQ ID NO: 50 | αmCSF1R VL | EIVLTQSPTTMAASPGEKVTITCRASSSTNYMSWYQQKSGASPKP |
| | | WIYETSKLASGVPDRFSGSGSGTSYSFTISSMEIEDAATYYCHQWS |
| | | STPLTFGSGTKLEIK |
| SEQ ID NO: 51 | R2a CL | RADAAPTVSIFPPSMEQLTSGGATVVCFVNNFYPRDISVKWKIDGS |
| | | EQRDGVLDSVTDQDSKDSTYSMSSTLSLTKVEYERHNLYTCEVVH |
| | | KTSSSPVVKSFNRNEC |
| SEQ ID NO: 52 | mFc Knob | TIKPCPPCKCPAPNLLGGPSVFIFPPKIKDVLMISLSPIVTCVVVDVS |
| | | EDDPDVQISWFVNNVEVHTAQTQTHREDYNSTLRVVSALPIQHQD |
| | | WMSGKEFKCKVNNKDLPAPIERTISKPKGSVRAPQVYVLPPCEEE |
| | | MTKKQVTLWCMVTDFMPEDIYVEWTNNGKTELNYKNIEPVLDS |
| | | DGSYFMYSKLRVEKKNWVERNSYSCSVVHEGLHNHHTTKSFSRT |
| | | PGK |
| SEQ ID NO: 53 | mFc Hole | TIKPCPPCKCPAPNLLGGPSVFIFPPKIKDVLMISLSPIVTCVVVDVS |
| | | EDDPDVQISWFVNNVEVHTAQTQTHREDYNSTLRVVSALPIQHQD |
| | | WMSGKEFKCKVNNKDLPAPIERTISKPKGSVRAPQVCVLPPPEEE |
| | | MTKKQVTLSCAVTDFMPEDIYVEWTNNGKIELNYKNIEPVLDSD |
| | | GSYFMVSKLRVEKKNWVERNSYSCSVVHEGLHNHHTTKSFSRTP |
| | | GK |
| SEQ ID NO: 54 | αhCCR2 | EVQLVESGGGLVKPGGSLRLSCAASGFTFSAYAMNWVRQAPGKG |
| | plozalizumab | LEWVGRIRTKNNNYATYYADSVKDRFTISRDDSKNTLYLQMNSL |
| | VH | KTEDTAVYYCTTFYGNGVWGQGTLVTVSS |
| SEQ ID NO: 55 | hCH1 | ASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNT |
| | | KVDKRVEPKSC |
| SEQ ID NO: 56 | hFc Knob | DKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLH |
| | | QDWLNGKEYKCKVSNKALPAPIEKTISKAKGQPREPQVYTLPPCR |
| | | EEMTKNQVSLWCLVKGFYPSDIAVEWESNGQPENNYKTTPPVLDS |
| | | DGSFFLYSKLTVDKSRWQQGNVFSCSVMHEALHNHYTQKSLSLSP |
| | | GX, wherein X is K or absent |
| SEQ ID NO: 57 | αhCCR2 | DVVMTQSPLSLPVTLGQPASISCKSSQSLLDSDGKTFLNWFQQRPG |
| | plozalizumab | QSPRRLIYLVSKLDSGVPDRFSGSGSGTDFTLKISRVEAEDVGVYY |
| | VL | CWQGTHFPYTFGQGTRLEIK |
| SEQ ID NO: 58 | hCL (kappa) | RTVAAPSVFIFPPSDEQLKSGTASVVCLLNNFYPREAKVQWKVDN |
| | | ALQSGNSQESVTEQDSKDSTYSLSSTLTLSKADYEKHKVYACEVT |
| | | HQGLSSPVTKSFNRGEC |
| SEQ ID NO: 59 | αhCCR2 D1 | EVQLVQSGAEVKKPGASVKVSCKASGYTFTGYHMHWVRQAPGQ |
| | VH | GLEWMGWINPNSGVTKYAQKFQGRVTMTRDTSINTAYMELSRLR |
| | | FDDTDVYYCATGGFGYWGEGTLVTVSS |
| SEQ ID NO: 60 | αhCCR2 D1 | LPVLTQPPSVSKGLRQTATLTCTGNSNNVGNQGAAWLQQHQGQP |
| | VL | PKLLSYRNHNRPSGVSERFSPSRSGDTSSLTITGLQPEDEADYYCLA |
| | | WDSSLRAFVFGTGTKLTVL |
| SEQ ID NO: 61 | hCL (lambda) | GQPKANPTVTLFPPSSEELQANKATLVCLISDFYPGAVTVAWKAD |
| | | GSPVKAGVETTKPSKQSNNKYAASSYLSLTPEQWKSHRSYSCQVT |
| | | HEGSTVEKTVAPIECS |
| SEQ ID NO: 62 | αhCCR2 42G7 | QVQLVQSGAEVKKPGASVKVSCKASGYTFSSYYMHWVRQAPGQ |
| | VH | GLEWMGIINPSGGNTSYAQKFQGRVTMTRDTSTSTVYMELSSLRS |
| | | EDTAVYYCARGGYQLPHGRARAFDMWGQGTMVTVSS |
| SEQ ID NO: 63 | αhCCR2 42G7 | AIRMTQSPLSLPVTLGQPASISCTSSQSLVYRDGTTYLNWFQQRPG |
| | VL | QSPRRLIYKVSNRDSGVPDRFTGSGSGTTFTLTISRVEAEDVGIYYC |
| | | MQGTHWPLTFGQGTKVEIK |
| SEQ ID NO: 64 | αhCCR2 43G12 | EVQLVESGGGLVQPGGSLRLSCVASGFTFSDYWMSWVRQAPGKG |
| | VH | LEWVANIKKDGSVNYYVDSVKGRFTISRDNAKNSLYLQMNSLRA |
| | | EDTAVYYCTRFDYWGQGTLVTVSS |
| SEQ ID NO: 65 | αhCCR2 43G12 | QAGLTQPPSVSKGLRQTATLTCTGNSNNVGNQGAAWLQQHQGHP |
| | VL | PKLLFYRNNNRASGISERLSASRSGNTASLTITGLQPEDEADYYCL |
| | | TWDSSLSVVVFGGGTKLTVL |
| SEQ ID NO: 66 | αhCSF1R | QVQLVQSGAEVKKPGASVKVSCKASGYTFTSYDISWVRQAPGQG |
| | emactuzumab | LEWMGVIWTDGGTNYAQKLQGRVTMTTDTSTSTAYMELRSLRSD |
| | VH | DTAVYYCARDQRLYFDVWGQGTTVTVSS |
| SEQ ID NO: 67 | αhC5F1R | DIQMTQSPSSLSASVGDRVTITCRASEDVNTYVSWYQQKPGKAPK |
| | emactuzumab | LLIYAASNRYTGVPSRFSGSGSGTDFTLTISSLQPEDFATYYCQQSF |
| | VL | SYPTFGQGTKLEIK |
| SEQ ID NO: 68 | hFc Hole | DKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLH |
| | | QDWLNGKEYKCKVSNKALPAPIEKTISKAKGQPREPQVCTLPPSR |
| | | EEMTKNQVSLSCAVKGFYPSDIAVEWESNGQPENNYKTTPPVLDS |
| | | DGSFFLVSKLTVDKSRWQQGNVFSCSVMHEALHNHYTQKSLSLSP |
| | | GX, wherein X is K or absent |
| SEQ ID NO: 69 | αhCSF1R | QVQLVQSGAEVKKPGSSVKVSCKASGYTFTDNYMIWVRQAPGQG |
| | cabiralizumab | LEWMGDINPYNGGTTFNQKFKGRVTITADKSTSTAYMELSSLRSE |
| | VH | DTAVYYCARESPYFSNLYVMDYWGQGTLVTVSS |
| SEQ ID NO: 70 | αhC5F1R | EIVLTQSPATLSLSPGERATLSCKASQSVDYDGDNYMNWYQQKPG |
| | cabiralizumab | QAPRLLIYAASNLESGIPARFSGSGSGTDFTLTISSLEPEDFAVYYC |
| | VL | HLSNEDLSTFGGGTKVEIK |

TABLE 5
Protein sequences for full heavy and light chains.

| Full length | Variable | Constant | Fc |
|---|---|---|---|
| SEQ ID NO: 71 | SEQ ID NO: 44 | SEQ ID NO: 46 | SEQ ID NO: 52 |
| SEQ ID NO: 72 | SEQ ID NO: 45 | SEQ ID NO: 47 | |
| SEQ ID NO: 73 | SEQ ID NO: 48 | SEQ ID NO: 49 | SEQ ID NO: 53 |
| SEQ ID NO: 74 | SEQ ID NO: 50 | SEQ ID NO: 51 | |
| SEQ ID NO: 75 | SEQ ID NO: 54 | SEQ ID NO: 55 | SEQ ID NO: 56 |
| SEQ ID NO: 76 | SEQ ID NO: 57 | SEQ ID NO: 58 | |
| SEQ ID NO: 77 | SEQ ID NO: 59 | SEQ ID NO: 55 | SEQ ID NO: 56 |
| SEQ ID NO: 78 | SEQ ID NO: 60 | SEQ ID NO: 61 | |
| SEQ ID NO: 79 | SEQ ID NO: 62 | SEQ ID NO: 55 | SEQ ID NO: 56 |
| SEQ ID NO: 80 | SEQ ID NO: 63 | SEQ ID NO: 58 | |
| SEQ ID NO: 81 | SEQ ID NO: 64 | SEQ ID NO: 55 | SEQ ID NO: 56 |
| SEQ ID NO: 82 | SEQ ID NO: 65 | SEQ ID NO: 58 | |
| SEQ ID NO: 83 | SEQ ID NO: 66 | SEQ ID NO: 55 | SEQ ID NO: 68 |
| SEQ ID NO: 84 | SEQ ID NO: 67 | SEQ ID NO: 58 | |
| SEQ ID NO: 85 | SEQ ID NO: 69 | SEQ ID NO: 55 | SEQ ID NO: 68 |
| SEQ ID NO: 86 | SEQ ID NO: 70 | SEQ ID NO: 58 | |

TABLE 6
Amino acid sequences of the chains used to construct multispecific molecules.

| SEQ ID | |
|---|---|
| NO | Amino Acid Sequence |
| SEQ ID | QVQLQESGPGLVQPSQTLSLTCTVSGFSLTDFSVHWVRQPPGKGLEWMGRIRSEGNTDYNS |
| NO: 71 | ALKSRLSISRDTSKSQVFLKMNSLQTEDTAIYFCTRGDILGFGYWGQGVMVTVSSAQTTAP |
| | SVYPLAPGCGDTTSSTVTLGCLVKGYFPEPVTVTWNSGALSSDVHTFPAVLQSGLYTLTSS |
| | VTSSTWPSQTVTCNVAHPASSTKVDKKVERRTIKPCPPCKCPAPNLLGGPSVFIFPPKIKDVL |
| | MISLSPIVTCVVVDVSEDDPDVQISWFVNNVEVHTAQTQTHREDYNSTLRVVSALPIQHQD |
| | WMSGKEFKCKVNNKDLPAPIERTISKPKGSVRAPQVYVLPPCEEEMTKKQVTLWCMVTDF |
| | MPEDIYVEWTNNGKIELNYKNIEPVLDSDGSYFMYSKLRVEKKNWVERNSYSCSVVHEG |
| | LHNHHTTKSFSRTPGK |
| SEQ ID | DIVMTQSPLSVSVTPGESASISCRSSKSLLHFKGITFVYWYLQKPGQSPQLLIFRMSSLASGV |
| NO: 72 | PDRFSGSGSETDFTLKISRVEAEDVGTYYCGQLLENPYTFGAGTKLELKRADAAPTVSIFPPS |
| | MEQLTSGGATVVCFVNNFYPRDISVKWKIDGSEQRDGVLDSVTDQDSKDSTYSMSSTLSLT |
| | KVEYERHNLYTCEVVHKTSSSPVVKSFNRNEC |
| SEQ ID | QVQLQQSGAELVKPGSSVKISCKASGYTFTSNFMHWIKQQPGNGLEWIGWIYPGDGDFEY |
| NO: 73 | NQKFNGKATLTADKSSSTAYMQLSSLTSEDSAVYFCAVNYGGYVLDAWGQGASVTVSSA |
| | ETTAPSVYPLAPGTALKSNSMVTLGCLVKGYFPEPVTVTWNSGALSSGVHTFPAVLQSGLY |
| | TLTSSVTVPSSTWSSQAVTCNVAHPASSTKVDKKIVPRECNTIKPCPPCKCPAPNLLGGPSVF |
| | IFPPKIKDVLMISLSPIVTCVVVDVSEDDPDVQISWFVNNVEVHTAQTQTHREDYNSTLRVV |
| | SALPIQHQDWMSGKEFKCKVNNKDLPAPIERTISKPKGSVRAPQVCVLPPPEEEMTKKQVT |
| | LSCAVTDFMPEDIYVEWTNNGKTELNYKNIEPVLDSDGSYFMVSKLRVEKKNWVERNSY |
| | SCSVVHEGLHNHHTTKSFSRTPGK |
| SEQ ID | EIVLTQSPTTMAASPGEKVTITCRASSSTNYMSWYQQKSGASPKPWIYETSKLASGVPDRFS |
| NO: 74 | GSGSGTSYSFTISSMETEDAATYYCHQWSSTPLTFGSGTKLEIKRADAAPTVSIFPPSMEQLT |
| | SGGATVVCFVNNFYPRDISVKWKIDGSEQRDGVLDSVTDQDSKDSTYSMSSTLSLTKVEYE |
| | RHNLYTCEVVHKTSSSPVVKSFNRNEC |
| SEQ ID | EVQLVESGGGLVKPGGSLRLSCAASGFTFSAYAMNWVRQAPGKGLEWVGRIRTKNNNYA |
| NO: 75 | TYYADSVKDRFTISRDDSKNTLYLQMNSLKIEDTAVYYCTTFYGNGVWGQGTLVTVSSAS |
| | TKGPSVFPLAPSSKSTSGGTAALGCLVKDYFPEPVTVSWNSGALTSGVHTFPAVLQSSGLYS |
| | LSSVVTVPSSSLGTQTYICNVNHKPSNTKVDKRVEPKSCDKTHTCPPCPAPELLGGPSVFLFP |
| | PKPKDTLMISRTPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPCREEMTKNQVSL |
| | WCLVKGFYPSDIAVEWESNGQPENNYKTTPPVLDSDGSFFLYSKLTVDKSRWQQGNVFSC |
| | SVMHEALHNHYTQKSLSLSPGX, wherein X is K or absent |
| SEQ ID | DVVMTQSPLSLPVTLGQPASISCKSSQSLLDSDGKTFLNWFQQRPGQSPRRLIYLVSKLDSG |
| NO: 76 | VPDRFSGSGSGTDFTLKISRVEAEDVGVYYCWQGTHFPYTFGQGTRLEIKRTVAAPSVFIFP |
| | PSDEQLKSGTASVVCLLNNFYPREAKVQWKVDNALQSGNSQESV1EQDSKDSTYSLSSTLT |
| | LSKADYEKHKVYACEVTHQGLSSPVTKSFNRGEC |
| SEQ ID | EVQLVQSGAEVKKPGASVKVSCKASGYTFTGYHMHWVRQAPGQGLEWMGWINPNSGVT |
| NO: 77 | KYAQKFQGRVTMTRDTSINTAYMELSRLRFDDTDVYYCATGGFGYWGEGTLVTVSSAST |
| | KGPSVFPLAPSSKSTSGGTAALGCLVKDYFPEPVTVSWNSGALTSGVHTFPAVLQSSGLYSL |
| | SSVVTVPSSSLGTQTYICNVNHKPSNTKVDKRVEPKSCDKTHTCPPCPAPELLGGPSVFLFPP |
| | KPKDTLMISRTPEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPCREEMTKNQVSL |
| | WCLVKGFYPSDIAVEWESNGQPENNYKTTPPVLDSDGSFFLYSKLTVDKSRWQQGNVFSC |
| | SVMHEALHNHYTQKSLSLSPGX, wherein X is K or absent |
| SEQ ID | LPVLTQPPSVSKGLRQTATLTCTGNSNNVGNQGAAWLQQHQGQPPKLLSYRNHNRPSGVS |
| NO: 78 | ERFSPSRSGDTSSLTITGLQPEDEADYYCLAWDSSLRAFVFGTGTKLTVLGQPKANPTVTLF |
| | PPSSEELQANKATLVCLISDFYPGAVTVAWKADGSPVKAGVETTKPSKQSNNKYAASSYLS |
| | LTPEQWKSHRSYSCQVTHEGSTVEKTVAPTECS |
| SEQ ID | QVQLVQSGAEVKKPGASVKVSCKASGYTFSSYYMHWVRQAPGQGLEWMGIINPSGGNTS |
| NO: 79 | YAQKFQGRVTMTRDTSTSTVYMELSSLRSEDTAVYYCARGGYQLPHGRARAFDMWGQGT |
| | MVTVSSASTKGPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKRVEPKSCDKTHTCPPCPAPEL |
| | LGGPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPCRE |
| | EMTKNQVSLWCLVKGFYPSDIAVEWESNGQPENNYKTTPPVLDSDGSFFLYSKLTVDKSR |
| | WQQGNVFSCSVMHEALHNHYTQKSLSLSPGX, wherein X is K or absent |
| SEQ ID | AIRMTQSPLSLPVTLGQPASISCTSSQSLVYRDGTTYLNWFQQRPGQSPRRLIYKVSNRDSG |
| NO: 80 | VPDRFTGSGSGTTFTLTISRVEAEDVGIYYCMQGTHWPLTFGQGTKVEIKRTVAAPSVFIFPP |
| | SDEQLKSGTASVVCLLNNFYPREAKVQWKVDNALQSGNSQESVIEQDSKDSTYSLSSTLTL |
| | SKADYEKHKVYACEVTHQGLSSPVTKSFNRGEC |
| SEQ ID | EVQLVESGGGLVQPGGSLRLSCVASGFTFSDYWMSWVRQAPGKGLEWVANIKKDGSVNY |
| NO: 81 | YVDSVKGRFTISRDNAKNSLYLQMNSLRAEDTAVYYCTRFDYWGQGTLVTVSSASTKGPS |
| | VFPLAPSSKSTSGGTAALGCLVKDYFPEPVTVSWNSGALTSGVHTFPAVLQSSGLYSLSSVV |
| | TVPSSSLGTQTYICNVNHKPSNTKVDKRVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPCREEMTKNQVSLWCL |
| | VKGFYPSDIAVEWESNGQPENNYKTTPPVLDSDGSFFLYSKLTVDKSRWQQGNVFSCSVM |
| | HEALHNHYTQKSLSLSPGX, wherein X is K or absent |
| SEQ ID | QAGLTQPPSVSKGLRQTATLTCTGNSNNVGNQGAAWLQQHQGHPPKLLFYRNNNRASGIS |
| NO: 82 | ERLSASRSGNTASLTITGLQPEDEADYYCLTWDSSLSVVVFGGGTKLTVLGQPKANPTVTL |
| | FPPSSEELQANKATLVCLISDFYPGAVTVAWKADGSPVKAGVETTKPSKQSNNKYAASSYL |
| | SLTPEQWKSHRSYSCQVTHEGSTVEKTVAPTECS |
| SEQ ID | QVQLVQSGAEVKKPGASVKVSCKASGYTFTSYDISWVRQAPGQGLEWMGVIWTDGGTNY |
| NO: 83 | AQKLQGRVTMTTDTSTSTAYMELRSLRSDDTAVYYCARDQRLYFDVWGQGTTVTVSSAS |
| | TKGPSVFPLAPSSKSTSGGTAALGCLVKDYFPEPVTVSWNSGALTSGVHTFPAVLQSSGLYS |
| | LSSVVTVPSSSLGTQTYICNVNHKPSNTKVDKRVEPKSCDKTHTCPPCPAPELLGGPSVFLFP |
| | PKPKDTLMISRTPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVCTLPPSREEMTKNQVSL |
| | SCAVKGFYPSDIAVEWESNGQPENNYKTTPPVLDSDGSFFLVSKLTVDKSRWQQGNVFSCS |
| | VMHEALHNHYTQKSLSLSPGX, wherein X is K or absent |
| SEQ ID | DIQMTQSPSSLSASVGDRVTITCRASEDVNTYVSWYQQKPGKAPKLLIYAASNRYTGVPSR |
| NO: 84 | FSGSGSGTDFTLTISSLQPEDFATYYCQQSFSYPTFGQGTKLEIKRTVAAPSVFIFPPSDEQLK |
| | SGTASVVCLLNNFYPREAKVQWKVDNALQSGNSQESVTEQDSKDSTYSLSSTLTLSKADY |
| | EKHKVYACEVTHQGLSSPVTKSFNRGEC |
| SEQ ID | QVQLVQSGAEVKKPGSSVKVSCKASGYTFTDNYMIWVRQAPGQGLEWMGDINPYNGGTT |
| NO: 85 | FNQKFKGRVTITADKSTSTAYMELSSLRSEDTAVYYCARESPYFSNLYVMDYWGQGTLVT |
| | VSSASTKGPSVFPLAPSSKSTSGGTAALGCLVKDYFPEPVTVSWNSGALTSGVHTFPAVLQS |
| | SGLYSLSSVVTVPSSSLGTQTYICNVNHKPSNTKVDKRVEPKSCDKTHTCPPCPAPELLGGP |
| | SVFLFPPKPKDTLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVCTLPPSREEMTK |
| | NQVSLSCAVKGFYPSDIAVEWESNGQPENNYKTTPPVLDSDGSFFLVSKLTVDKSRWQQG |
| | NVFSCSVMHEALHNHYTQKSLSLSPGX, wherein X is K or absent |
| SEQ ID | EIVLTQSPATLSLSPGERATLSCKASQSVDYDGDNYMNWYQQKPGQAPRLLIYAASNLESG |
| NO: 86 | IPARFSGSGSGTDFTLTISSLEPEDFAVYYCHLSNEDLSTFGGGTKVEIKRTVAAPSVFIFPPS |
| | DEQLKSGTASVVCLLNNFYPREAKVQWKVDNALQSGNSQESVTEQDSKDSTYSLSSTLTLS |
| | KADYEKHKVYACEVTHQGLSSPVTKSFNRGEC |

TABLE 7
Sequences used to generate aCCR2/aCSFlR multispecific molecules.

| Multispecific | | | | |
|---|---|---|---|---|
| Molecule | Heavy Chain 1 | Light Chain 1 | Heavy Chain 2 | Light Chain 2 |
| 1 | SEQ ID NO: 71 | SEQ ID NO: 72 | SEQ ID NO: 73 | SEQ ID NO: 74 |
| 2 | SEQ ID NO: 75 | SEQ ID NO: 76 | SEQ ID NO: 83 | SEQ ID NO: 84 |
| 3 | SEQ ID NO: 75 | SEQ ID NO: 76 | SEQ ID NO: 85 | SEQ ID NO: 86 |
| 4 | SEQ ID NO: 77 | SEQ ID NO: 78 | SEQ ID NO: 83 | SEQ ID NO: 84 |
| 5 | SEQ ID NO: 77 | SEQ ID NO: 78 | SEQ ID NO: 85 | SEQ ID NO: 86 |
| 6 | SEQ ID NO: 79 | SEQ ID NO: 80 | SEQ ID NO: 83 | SEQ ID NO: 84 |
| 7 | SEQ ID NO: 79 | SEQ ID NO: 80 | SEQ ID NO: 85 | SEQ ID NO: 86 |
| 8 | SEQ ID NO: 81 | SEQ ID NO: 82 | SEQ ID NO: 83 | SEQ ID NO: 84 |
| 9 | SEQ ID NO: 81 | SEQ ID NO: 82 | SEQ ID NO: 85 | SEQ ID NO: 86 |

### Example 2. UniTI-01 Binding to Cells Expressing mCCR2 Alone, mCSF1R Alone, or Both mCCR2 and mCSF1R

In this and the next few examples, multispecific molecule #1 (also referred to as UniTI-01) shown in Table 7 was characterized. UniTI-01 is an anti-CCR2/anti-CSF1R bispecific antibody. The variable region and full length sequences of UniTI-01 are also provided in Table 11. In a few examples, the bispecific antibody UniTI-01 was compared against an anti-CCR2 bivalent monospecific antibody or an anti-CSF1R bivalent monospecific antibody.

TABLE 11
Sequences of an anti-CCR2/anti-CSF1R bispecific antibody molecule

| SEQ ID | | |
|---|---|---|
| NO | Description | Sequence |
| SEQ ID | αCCR2 MC12 | QVQLQESGPGLVQPSQTLSLTCTVSGFSLTDFSVHWVRQPPGKGLEWMG |
| NO: 44 | VH | RIRSEGNTDYNSALKSRLSISRDTSKSQVFLKMNSLQTEDTAIYFCTRGDI |
| | | LGFGYWGQGVMVTVSS |
| SEQ ID | αCCR2 MC12 | DIVMTQSPLSVSVTPGESASISCRSSKSLLHFKGITFVYWYLQKPGQSPQL |
| NO: 45 | VL | LIFRMSSLASGVPDRFSGSGSETDFTLKISRVEAEDVGTYYCGQLLENPYT |
| | | FGAGTKLELK |
| SEQ ID | Full length | QVQLQESGPGLVQPSQTLSLTCTVSGFSLTDFSVHWVRQPPGKGLEWMG |
| NO: 71 | αCCR2 MC12 | RIRSEGNTDYNSALKSRLSISRDTSKSQVFLKMNSLQTEDTAIYFCTRGDI |
| | heavy chain | LGFGYWGQGVMVTVSSAQTTAPSVYPLAPGCGDTTSSTVTLGCLVKGY |
| | | FPEPVTVTWNSGALSSDVHTFPAVLQSGLYTLTSSVTSSTWPSQTVTCNV |
| | | AHPASSTKVDKKVERRTIKPCPPCKCPAPNLLGGPSVFIFPPKIKDVLMISL |
| | | SPIVTCVVVDVSEDDPDVQISWFVNNVEVHTAQTQTHREDYNSTLRVVS |
| | | ALPIQHQDWMSGKEFKCKVNNKDLPAPIERTISKPKGSVRAPQVYVLPPC |
| | | EEEMTKKQVTLWCMVTDFMPEDIYVEWTNNGKTELNYKNTEPVLDSD |
| | | GSYFMYSKLRVEKKNWVERNSYSCSVVHEGLHNHHTTKSFSRTPGK |
| SEQ ID | Full length | DIVMTQSPLSVSVTPGESASISCRSSKSLLHFKGITFVYWYLQKPGQSPQL |
| NO: 72 | αCCR2 MC12 | LIFRMSSLASGVPDRFSGSGSETDFTLKISRVEAEDVGTYYCGQLLENPYT |
| | light chain | FGAGTKLELKRADAAPTVSIFPPSMEQLTSGGATVVCFVNNFYPRDISVK |
| | | WKIDGSEQRDGVLDSVTDQDSKDSTYSMSSTLSLTKVEYERHNLYTCEV |
| | | VHKTSSSPVVKSFNRNEC |
| SEQ ID | αmCSF1R VH | QVQLQQSGAELVKPGSSVKISCKASGYTFTSNFMHWIKQQPGNGLEWIG |
| NO: 48 | | WIYPGDGDIEYNQKFNGKATLTADKSSSTAYMQLSSLTSEDSAVYFCAV |
| | | NYGGYVLDAWGQGASVTVSS |
| SEQ ID | αmCSF1R VL | EIVLTQSPTTMAASPGEKVTITCRASSSTNYMSWYQQKSGASPKPWIYET |
| NO: 50 | | SKLASGVPDRFSGSGSGTSYSFTISSMETEDAATYYCHQWSSTPLTFGSG |
| | | TKLEIK |
| SEQ ID | Full length | QVQLQQSGAELVKPGSSVKISCKASGYTFTSNFMHWIKQQPGNGLEWIG |
| NO: 73 | αmCSF1R | WIYPGDGDIEYNQKFNGKATLTADKSSSTAYMQLSSLTSEDSAVYFCAV |
| | heavy chain | NYGGYVLDAWGQGASVTVSSAETTAPSVYPLAPGTALKSNSMVTLGCL |
| | | VKGYFPEPVTVTWNSGALSSGVHTFPAVLQSGLYTLTSSVTVPSSTWSSQ |
| | | AVTCNVAHPASSTKVDKKIVPRECNTIKPCPPCKCPAPNLLGGPSVFIFPP |
| | | KIKDVLMISLSPIVTCVVVDVSEDDPDVQISWFVNNVEVHTAQTQTHRED |
| | | YNSTLRVVSALPIQHQDWMSGKEFKCKVNNKDLPAPIERTISKPKGSVRA |
| | | PQVCVLPPPEEEMTKKQVTLSCAVTDFMPEDIYVEWTNNGKTELNYKNT |
| | | EPVLDSDGSYFMVSKLRVEKKNWVERNSYSCSVVHEGLHNHHTTKSFS |
| | | RTPGK |
| SEQ ID | Full length | EIVLTQSPTTMAASPGEKVTITCRASSSTNYMSWYQQKSGASPKPWIYET |
| NO: 74 | αmCSF1R | SKLASGVPDRFSGSGSGTSYSFTISSMETEDAATYYCHQWSSTPLTFGSG |
| | light chain | TKLEIKRADAAPTVSIFPPSMEQLTSGGATVVCFVNNFYPRDISVKWKID |
| | | GSEQRDGVLDSVTDQDSKDSTYSMSSTLSLTKVEYERHNLYTCEVVHKT |
| | | SSSPVVKSFNRNEC |

ExpiCHO cells were transiently transfected with mouse CCR2, mouse CSF1R, or both mouse CCR2 and CSF1R, according to the manufacturer's instructions. In brief, the transfections were performed with ExpiCHO cells at a cell density of 5.6-6.3×10⁶ cells/mL and at least 95% viability. For each transfection, 25 μg of DNA was diluted with 1 mL of OptiPro SFM (Gibco 12309050) and filtered using spin-X centrifuge tube filters (Corning 8160). A solution of 920 L of OptiPro and 80 μL of expifectamine was added to the filtered DNA, incubated for 1 minute at room temperature, and then added to the cells. On day 1 of the transfection, the cells were enhanced using 150 μL of ExpiCHO enhancer.

On day 2 of the transfection, the cells were washed with PBS containing 1% BSA (Sigma) and used to set 96-well V-bottom plates (Biotix AP-0350-9CVS) with 100,000 cells/well. UniTI-01 was added to the cells in serial dilutions and incubated for 1 hour at 4° C. The plates were washed twice with PBS containing 1% BSA. The secondary antibody was a 1:500 dilution of goat anti-mouse Fc biotin antibody (Invitrogen Cat. No. 31805), and incubated with the cells for 45 minutes at 4° C. The plates were washed twice with PBS containing 1% BSA. For detection, 1.56×10⁻³ μg of streptavidin-PE (eBioscience Cat. No. 12-4317-87) was used, per well, and incubated for 1 hour at 4° C. The plates were read on a CytoFLEX S (Beckman Coulter). Data were calculated as the median fluorescence intensity of the PE-positive population vs. the median fluorescence intensity of the PE-negative population. The data was normalized for the percent of total median fluorescence intensity.

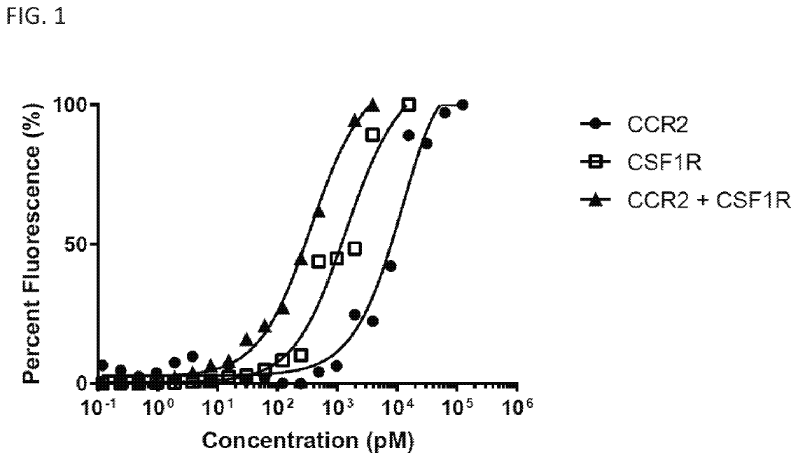

Without wishing to be bound by theory, UniTI-01 may preferentially bind cells expressing both CCR2 and CSF1R relative to cells that express either CCR2 or CSF1R. Consistent with the hypothesis that dual target binding increases the avidity of UniTI-01 for the target cell, UniTI-01 exhibited enhanced binding to CCR2 and CSF1R double positive cells, relative to single positive cells (FIG. 1). UniTI-01 showed binding with an EC₅₀ of 15 nM to cells expressing only CCR2. For cells expressing only CSF1R, UniTI-01 had an EC₅₀ of 1 nM. UniTI-01 displayed an EC50 of 400 pM to cells that expressed both CCR2 and CSF1R.

### Example 3. UniTI-01 Inhibits MCP1-Induced Migration of Bone Marrow Derived Monocytes In Vitro

Mouse bone marrow cells were isolated from femurs of healthy Balb/c mice and differentiated into monocytes in the presence of mCSF1 for four days. Monocyte differentiation was assessed by flow-cytometric analysis of CCR2 and CSF1R expression on hematopoietic cells. The differentiated cells were counted, and cultured with different concentrations of Isotype (mIgG2a), anti-CCR2 and UniTI-01 respectively for 30 minutes at 37° C. Antibody treated cells were subsequently added on the upper chamber in the transwell insert plates, which contained MCP1 (CCL2) in the bottom chamber. MCP1 induced migration was assessed after collecting the media from bottom chamber of transwell plates and cell number enumeration was performed by flow-cytometric analysis.

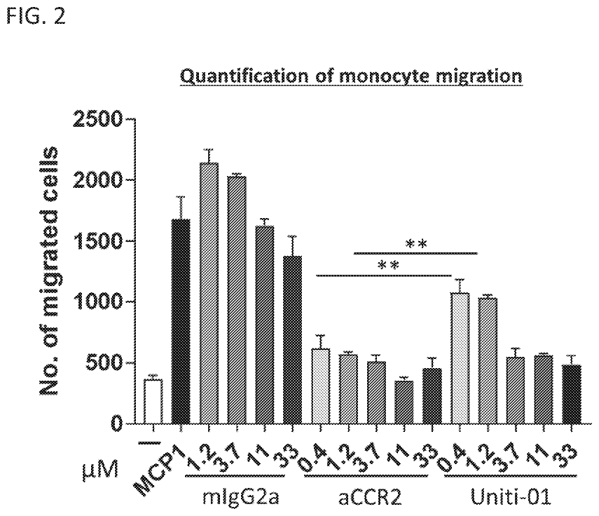

The anti-CCR2/anti-CSF1R bispecific antibody UniTI-01 inhibited the MCP1 induced migration of monocytes across the transwell in a dose dependent manner (FIG. 2). At doses 3.7 μM and above, monocyte migration was reduced to the levels observed in the absence of chemokine (FIG. 2). Similar results were obtained with anti-CCR2 antibody treatment, while treatment of cells with IgG2a did not influence migration (FIG. 2).

### Example 4. UniTI-01 Inhibits mCSF-1-Dependent Proliferation of Bone Marrow-Derived Macrophages In Vitro

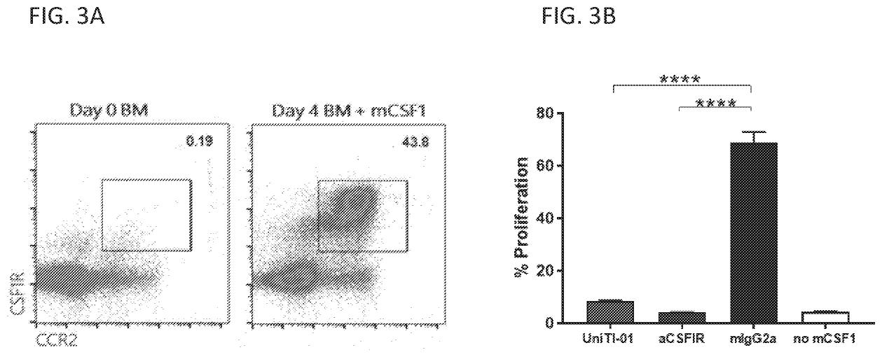

Mouse bone marrow cells were isolated from femurs of healthy Balb/c mice and differentiated into monocytes in the presence of mCSF1 for four days. Flow cytometry analysis showed bone marrow cells did not appreciably express CCR2 (FIG. 3A; left panel) at day 0. However, after 4 days of cell culture in the presence of mCSF1, a significant portion of bone marrow cell differentiated into monocytes which displayed CCR2 and CSF1R expression (FIG. 3A; right panel). Monocytes were counted and plated in 96 well plates in the presence of mCSF1. UniTI-01, anti-CSF1R or mIgG2a antibody was added to the cells containing mCSF1. After 72 hours of incubation at 37° C., cell proliferation metabolic activity was assessed by colorimetric reading at OD570 following manufacturer's protocol for the MTT assay kit.

mCSF1 induced differentiation of monocytes to macrophages, visualized as long fibroblastic cells under the microscope. UniTI-01 and anti-CSF1R antibodies prevented the proliferation of macrophages in the presence of mCSF1 (FIG. 3B).

### Example 5. UniTI-01 does not Inhibit mCSF-1-Dependent Bone Marrow-Derived Monocyte Differentiation In Vitro

Bone marrow cells were extracted from both femur and tibia of a naive (non-tumor bearing) Balb/c mouse. UniTI-01, anti-CSF1R or mIgG2 antibodies were pre-incubated with freshly isolated bone marrow cells for 30 minutes at 37° C. before the addition of mCSF-1 to allow monocyte differentiation. After 4 days of incubation at 37° C., cells were collected for flow cytometry staining to identify differentiated monocytes. Cells were stained with fluorescent-labeled antibodies for 15 minutes at 4° C. followed by flow cytometry analysis. Monocytes are gated as Live, CD45+, CD11b+, Ly6C+, Ly6G− cells.

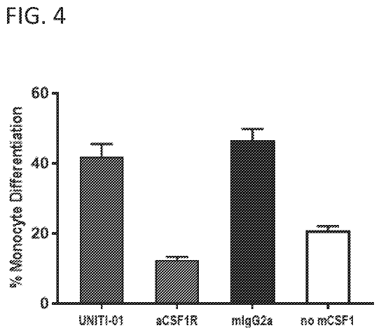

Consistent with the observation that bone marrow precursor cells did not significantly express CCR2 at day 0 (FIG. 3A), the anti-CCR2/anti-CSF1R bispecific antibody UniTI-01 did not inhibit mCSF-1-dependent bone marrow-derived monocyte differentiation in vitro after 4 days of cell culture (FIG. 4). In contrast, the anti-CSF1R bivalent monospecific antibody significantly inhibited mCSF-1-dependent bone marrow-derived monocyte differentiation in vitro (FIG. 4).

### Example 6. UniTI-01 Specifically Binds to Primary Intratumoral M-MDSCs and M2-Like Macrophages In Vitro

LLC tumors, grown in B6− albino mice, were harvested at a volume of 500-800 mm³ and dissociated using liberase DL+Dnase I for 30 minutes at 37° C., followed by the dissociation program m_imptumor_01 on the GentleMacs. Single cell suspensions were filtered through a 70 μm strainer and total cells were stained with fluorescently labeled antibodies. For this binding study, 100 μg of UniTI-01 was labeled using the Alexa Fluor 647 Antibody Labeling Kit (ThermoFisher Scientific), the concentration of labeled antibody was determined by Nanodrop, and the indicated serial dilutions (5 uM, 1 uM, 0.1 uM, 0.01 uM) were made in Facs buffer. M-MDSCs were gated by live CD45+CD11b+Ly6ChighLy6G−, M2 macrophages were gated by live CD45+CD11b+F4/80+CD206+, CD3+ T cells were gated by live CD45+CD3+, and neutrophils were gated by live CD45+CD11b+Ly6G+. The cells were stained for 15 minutes on ice in the dark, stained with zombie violet for viability, and immediately acquired on the cytometer.

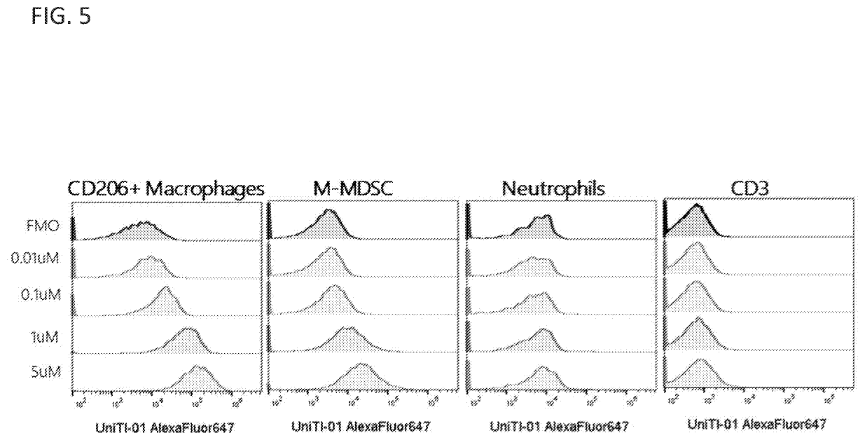

Concentration-dependent binding of UniTI-01 to M2 macrophages and M-MDSCs is shown in FIG. 5.

### Example 7. UniTI-01 Depletes Suppressive Myeloid Cells in Several Mouse Models In Vivo

Mice were injected either with MC38 colon cancer cell line (B6 albino mice) or EMT6 breast cancer cell line (BALB/c mice) and once tumors reached a volume of 150-200 mm³, the same mice were randomized and grouped into two arms. One arm received a treatment of 20 mg/kg UniTI-01 via ip route at a dose of 20 mg/kg on day 1, 4, 7 and 10 and the other received PBS (vehicle) at the same schedule. Twenty-four hours after the 4^{th} dose, tumors were harvested for flow cytometry analysis. Tumors were minced into ˜2 mm pieces and dissociated with liberase+dnase I for 30 minutes at 37° C., followed by using a 1 minute tumor blend program on the gentleMACs. Single cell suspensions were made by filtering through a 70 μM filter and counted. Cells were then stained for flow cytometry analysis. TAMs were gated by live CD45+CD11b+Ly6G−Ly6C-F4/80+ and M-MDSCs were gated by live CD45+CD11b+Ly6G−Ly6C^{high}. Each point represents a single mouse. Error bars represent the mean and standard error between individual mice. Statistics were calculated using Student's t test.

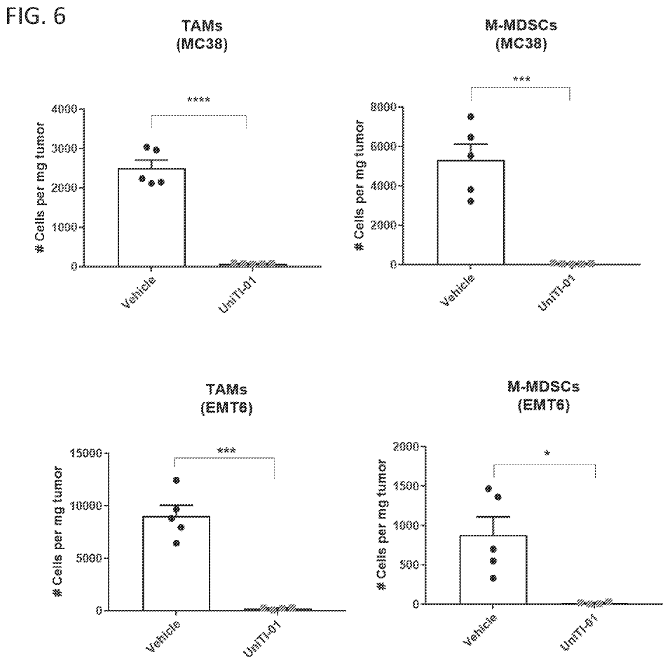

Consistent with the binding to M2 macrophages and M-MDSCs observed in Example 6, the anti-CCR2/anti-CSF1R bispecific antibody UniTI-01 reduced TAMs and M-MDSCs in both EMT6 and MC38 syngeneic tumor models (FIG. 6).

### Example 8. UniTI-01 Depletes Tumor-Associated Macrophages but Spares Healthy Tissue Macrophages

Balb/c mice were injected with EMT6 syngeneic breast cell line and once tumors reached a volume of 150-200 mm³, were randomized and grouped into three arms. One arm received a treatment of 20 mg/kg UniTI-01 via ip route at a dose of 20 mg/kg, the second arm received a treatment of 10 mg/kg anti-CSF1R antibody and the third arm received PBS on day 1, 4, 7 and 10. Twenty-four hours after the 4^{th} dose, mice were sacrificed and tumors and livers were harvested and formalin fixed and paraffin embedded. To detect the macrophage populations in liver and tumors, tissue sections were immunohistochemically stained with F4/80 antibody (Cell Signaling) and detected by Envision system. Approximately 10 regions of interest per tumor or liver section were analyzed by ImageJ software.

Without wishing to be bound by theory, UniTI-01 may preferentially bind to cells expressing both CCR2 and CSF1R relative to cells expressing either CCR2 or CSF1R, and may have less an effect on tissue-resident macrophages, such as the liver-resident Kupffer cells, which do not express CCR2, relative to tumor-associated macrophages which express both CCR2 and CSF1R.

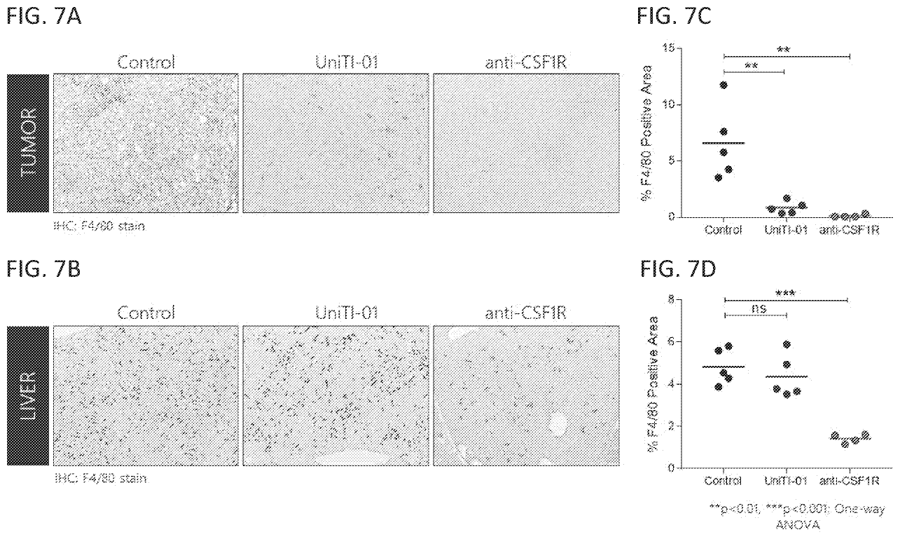

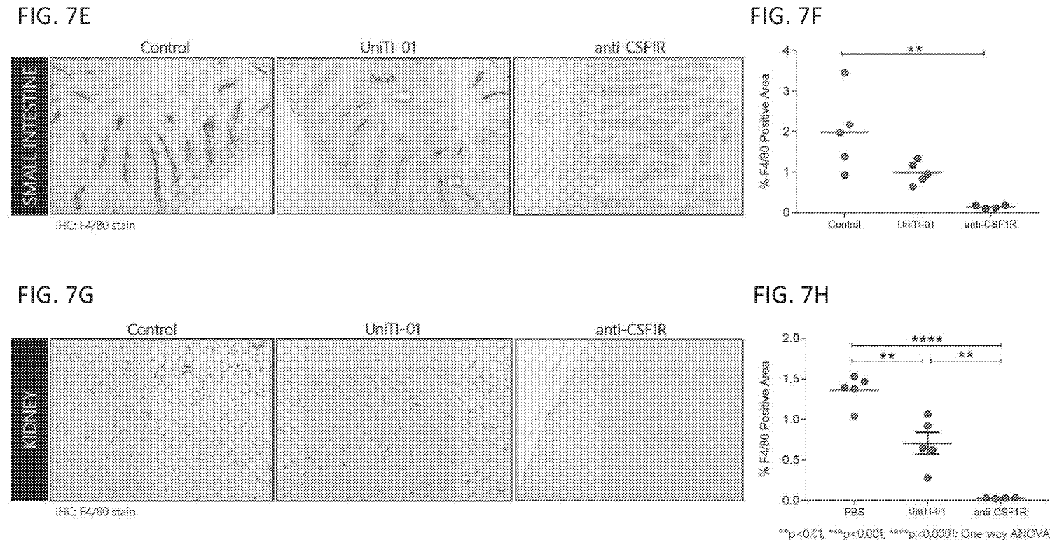

Consistent with the data described earlier (FIG. 6), the anti-CCR2/anti-CSF1R bispecific antibody UniTI-01 markedly depleted tumor-associated macrophages (FIGS. 7A and 7C). In contrast, UniTI-01 did not appreciably deplete tissue-resident macrophages in the liver (FIGS. 7B and 7D), whereas the anti-CSF1R bivalent monospecific antibody significantly reduced macrophages in both compartments (FIGS. 7A-7D).

Additional data show that compared with the anti-CSF1R bivalent monospecific antibody, UniTI-01 also spares healthy tissue macrophages in small intestine (FIGS. 7E and 7F) and kidney (FIGS. 7G and 7H).

### Example 9. UniTI-01 does not Inhibit CSF-1 Dependent Cell Survival in CCR2-Negative NFS-60 Cells In Vitro

CCR2-negative, CSF1R-positive NFS-60 cells were cultured in phenol-red-free media in the presence of UniTI-01, a monovalent monospecific anti-CSF1R antibody, a bivalent monospecific anti-CSF1R antibody, or a mIgG2 antibody for 30 minutes at 37° C. followed by the addition of mCSF-1 for cell survival. After 48 hours of incubation at 37° C., cell viability metabolic activity was assessed by colorimetric reading at OD570 following manufacturer's protocol for the MTT assay kit.

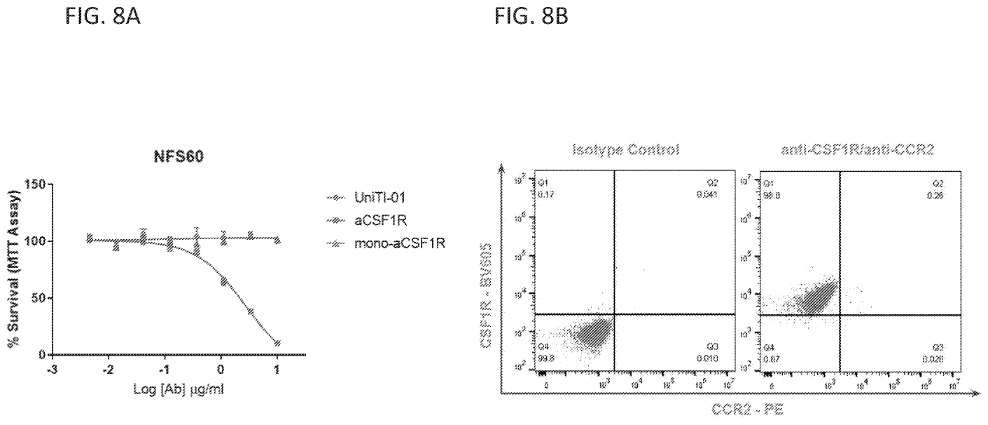

As shown in FIG. 8A, the anti-CSF1R bivalent monospecific antibody (αCSF1R) markedly reduced viability of NFS-60 cells at high antibody concentrations. In contrast, the anti-CCR2/anti-CSF1R bispecific antibody UniTI-01 or the monovalent monospecific anti-CSF1R antibody (mono-αCSF1R) did not inhibit CSF-1 dependent cell survival in CCR2-negative NFS-60 cells in vitro (FIG. 8A). Without wishing to be bound by theory, this data suggests that the anti-CCR2/anti-CSF1R bispecific antibody UniTI-01 may be less likely to inhibit CCR2-negative, CSF1R-positive cells, compared with an anti-CSF1R bivalent monospecific antibody, which is consistent with the results described in FIGS. 7B and 7D. To confirm NFS-60 did not express CCR2, cells were washed with BSA containing PBS buffer and fluorescent-labeled antibodies for CSF1R and CCR2 for 20 minutes at 4° C. followed by flow cytometry analysis. As shown in FIG. 8B, NFS-60 expressed CSF1R but not CCR2.

### Example 10. UniTI-01 Promotes CD8+ T Cell Infiltration in EMT6 Tumors In Vivo

Balb/c mice were injected with EMT6 syngeneic breast cell line and once tumors reached a volume of 150-200 mm³, were randomized and grouped into three arms. One arm received a treatment of 20 mg/kg UniTI-01 via ip route at a dose of 20 mg/kg, the second arm received a treatment of 10 mg/kg anti-PDL1 and the third arm received PBS on day 1, 4, 7 and 10. Twenty-four hours after the 4^{th} dose, mice were sacrificed and tumors were harvested for flow cytometry analysis. Tumors were minced into ˜2 mm pieces and dissociated with liberase+dnase I for 30 minutes at 37° C., followed by using a 1 minute tumor blend program on the gentleMACS. Single cell suspensions were made by filtering through a 70 μM filter and counted. Cells were then stained for flow cytometry analysis. CD8 T cells were gated by staining on live CD8 cells that expressed CD45 and CD3. Each point represents a single mouse tumor. Error bars represent the mean and standard error between individual mice. Statistics were calculated using one way ANOVA analysis.

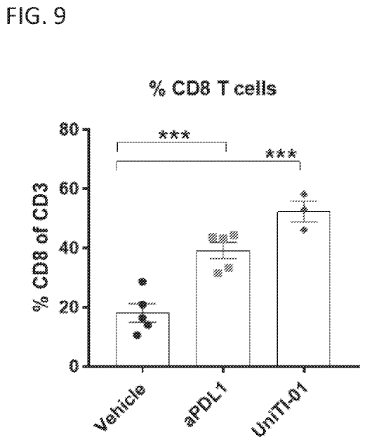

The anti-CCR2/anti-CSF1R bispecific antibody UniTI-01 significantly increased CD8+ T cell infiltration in EMT6 tumors in vivo (FIG. 9).

### Example 11. UniTI-01 Reduces Treg Frequency and Increases CD8 T Cells/Treg Ratio in EMT6 Tumors In Vivo

B6 albino mice were injected with MC38 syngeneic colon cell line and once tumors reached a volume of 150-200 mm³, were randomized and grouped into three arms. One arm 10 received a treatment of 20 mg/kg UniTI-01 via ip route at a dose of 20 mg/kg, the second arm received a treatment of 10 mg/kg anti-PDL1 and the third arm received PBS on day 1, 4, 7 and 10. Twenty-four hours after the 4^{th} dose, mice were sacrificed and tumors were harvested for immune profiling by Flow cytometry. Tumors were minced into ˜2 mm pieces and dissociated with liberase+dnase I for 30 minutes at 37° C., followed by using a 1 minute tumor blend program on the gentleMACs. Single cell suspensions were made by filtering through a 70 μM filter and counted. Cells were then stained for flow cytometry analysis. T regulatory cells were analyzed by gating on live CD4+ Foxp3+ T cells. In addition, CD8 T cells were gated by staining on live CD8 cells that expressed CD45 and CD3. Each point represents a single mouse tumor. Error bars represent the mean and standard error between individual mice. Statistics were calculated using one way ANOVA analysis.

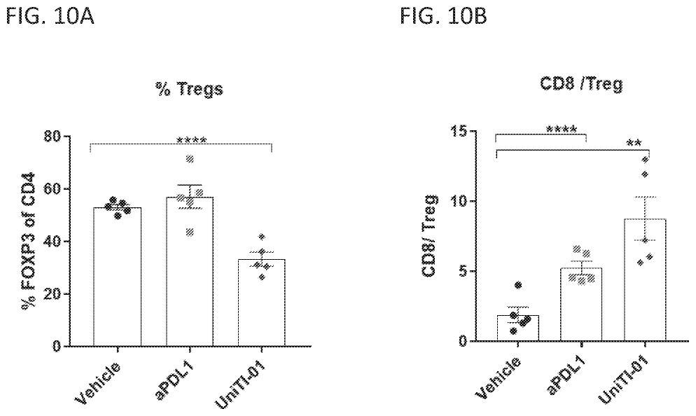

Treatment with UniTI-01 led to a greater reduction in Treg frequency and a greater increase in the CD8+ T cell/Treg ratio in the tumor, compared with treatment with the anti-PDL1 antibody (FIGS. 10A and 10B).

### Example 12. UniTI-01 Shows Antitumor Efficacy, Tumor Regressions and Enhanced Survival when Used in Combination with Anti-PDL1 Antibody

Tumor growth inhibition of UniTI-01 in combination with anti-PDL1 antibody was tested in a subcutaneous mouse syngeneic EMT6 breast cancer model. ˜8 week old female BALB/c (Jackson Labs) were acclimatized for 3 days prior to start of the studies. Mice were housed 5 animals per cage, and the disposable cages were placed in Innovive IVC mouse racks. EMT6 cells previously tested to be free from mouse pathogens (mouse CLEAR panel, Charles River Labs) were implanted subcutaneously on day 0 at a density of 0.5×10⁶ cells in the right flanks of BALB/c mice. Tumors were measured and recorded in two dimensions twice weekly using a digital caliper. Tumor volumes (mm³) were calculated using the formula width×width×length×0.52. Following tumor volume measurements on day 7 post implantation, mice were randomized and grouped into four arms according to a mean tumor volume of 77 mm³. One arm was treated with PBS, the second arm was treated with anti-PDL1 antibody at a dose of 10 mg/kg, the third arm treated with UniTI-01 at a dose of 20 mg/kg, and the fourth arm treated with a combination of anti-PDL1 antibody and UniTI-01. All treatments were via ip route and the schedules were twice weekly for up to four weeks of dosing. All the agents were formulated freshly in PBS prior to dosing. Tumor volume measurements continued up to 80 days. Tumor volume data is plotted as Mean±SEM. In addition, survival is recorded based on the time to reach the study endpoint of a TV of 2000 mm³ and plotted as a Kaplan Meier curve. Statistics were determined by Log− rank test and p− values are plotted.

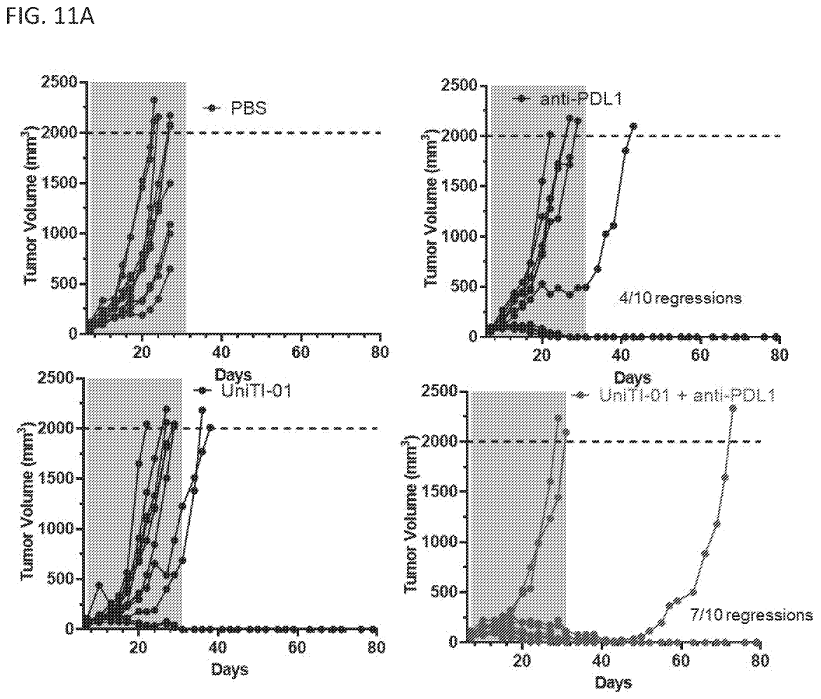

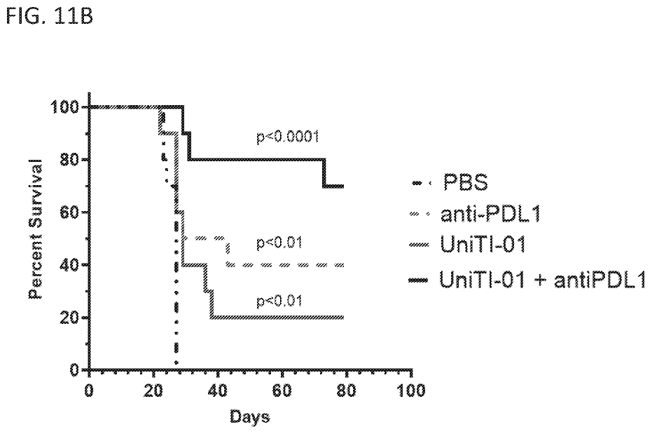

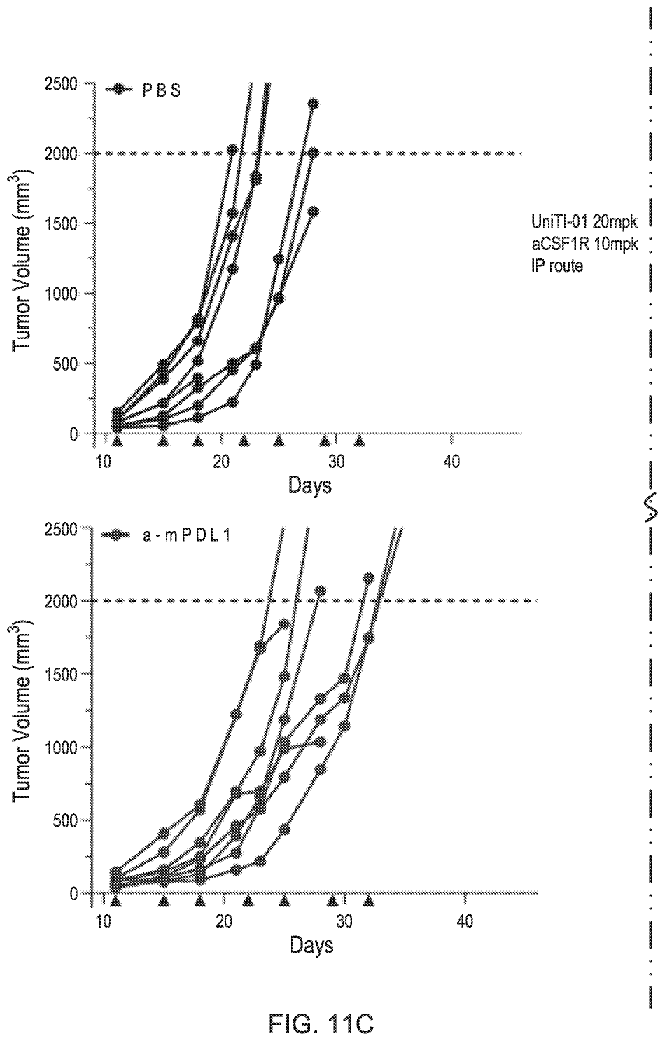

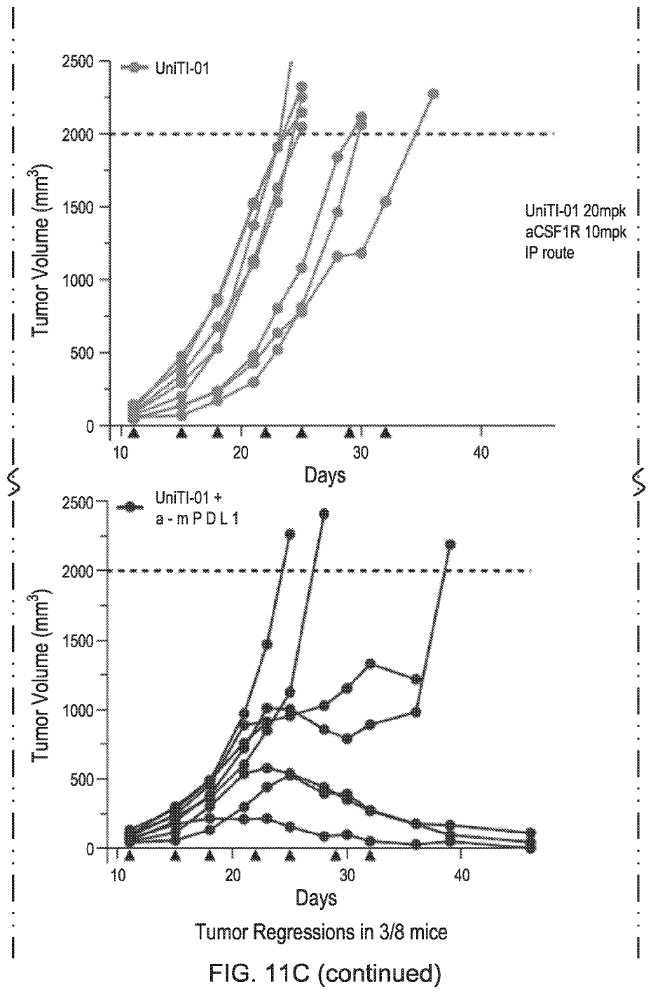

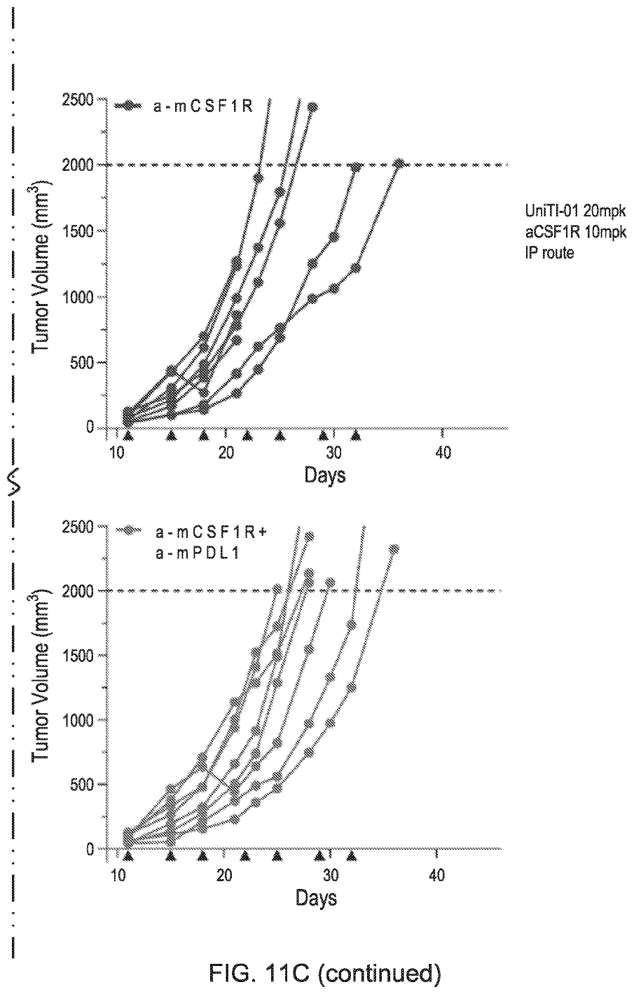

As shown in FIGS. 11A and 11B, treatment with UniTI-01 improved the survival of tumor-bearing animals. Combining UniTI-01 and an anti-PDL1 antibody further improved the antitumor efficacy, tumor regressions and survival in vivo (FIGS. 11A and 11B).

In a separate study using a MC38 colon mouse model, UniTI-01 shows superior anti-PDL1 combination benefit over anti-CSF1R antibody (FIG. 11C).

### Example 13. UniTI-01 Spares Osteoclasts when Compared to Anti-CSF1R Treatment

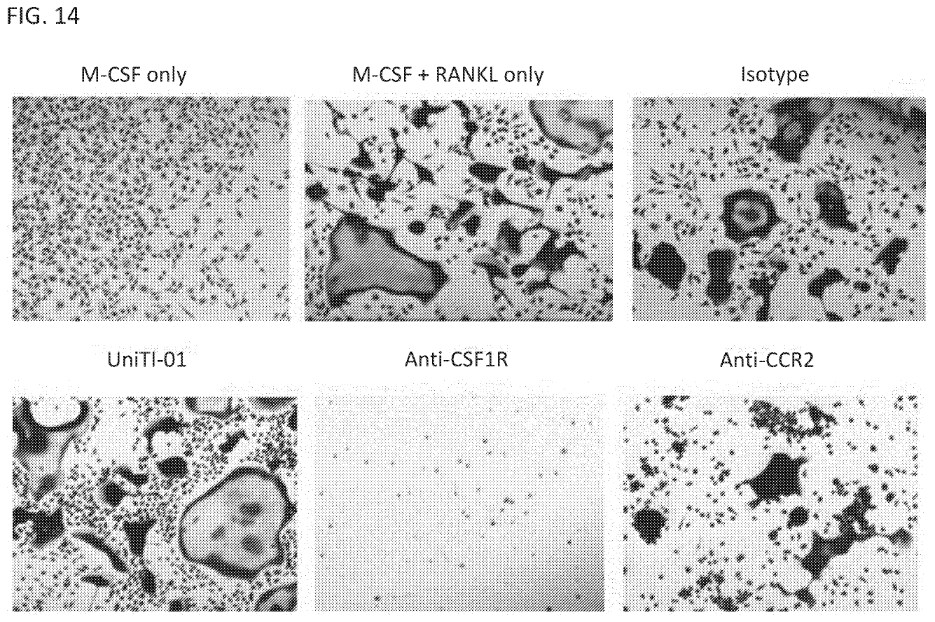

The next study relates to in vitro differentiation of osteoclasts from murine bone marrow cells with M-CSF and RANKL for 6 days, followed by fixation and TRAP staining. As shown in FIG. 14, UniTI-01 spares osteoclasts when compared with an anti-CSF1R bivalent monospecific antibody.

### Example 14. UniTI-01 Drives Depletion of M-MDSC and TAMs in Tumors

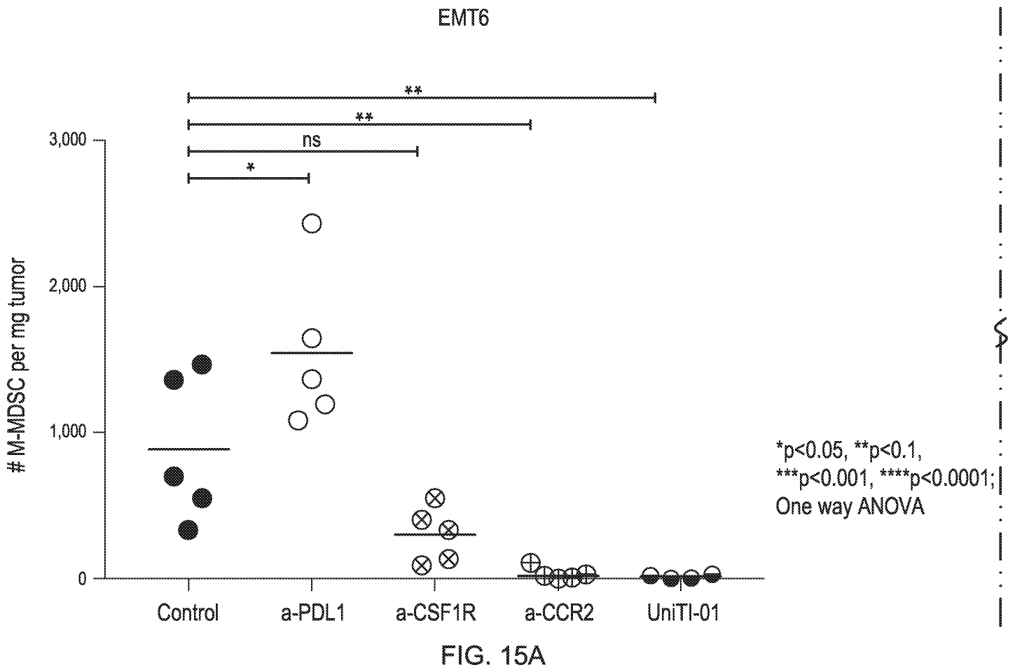

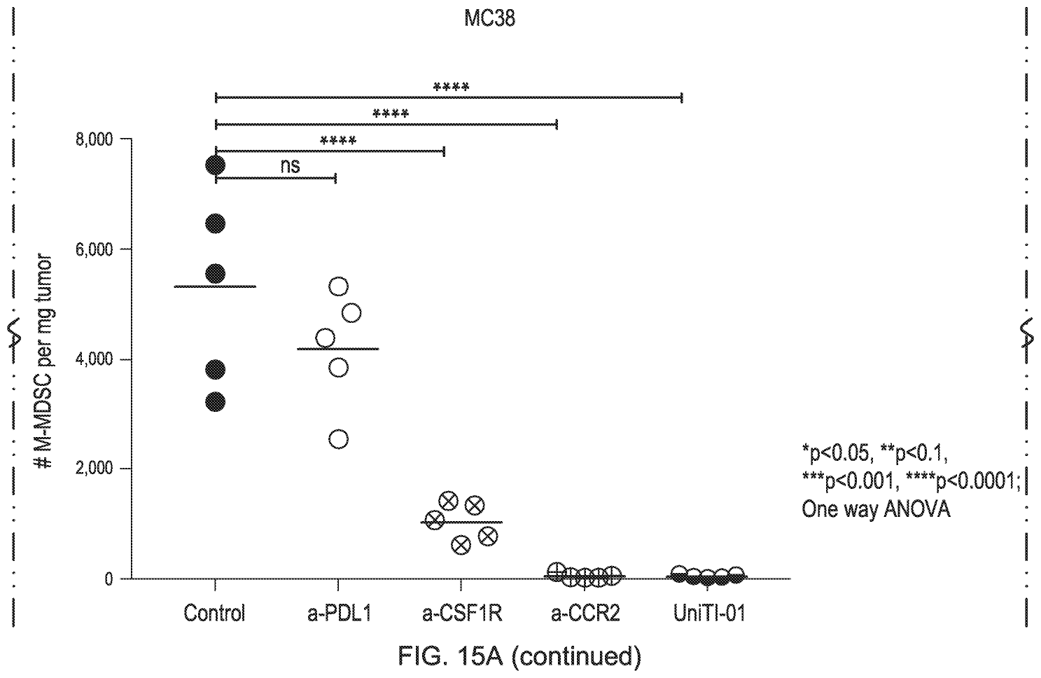

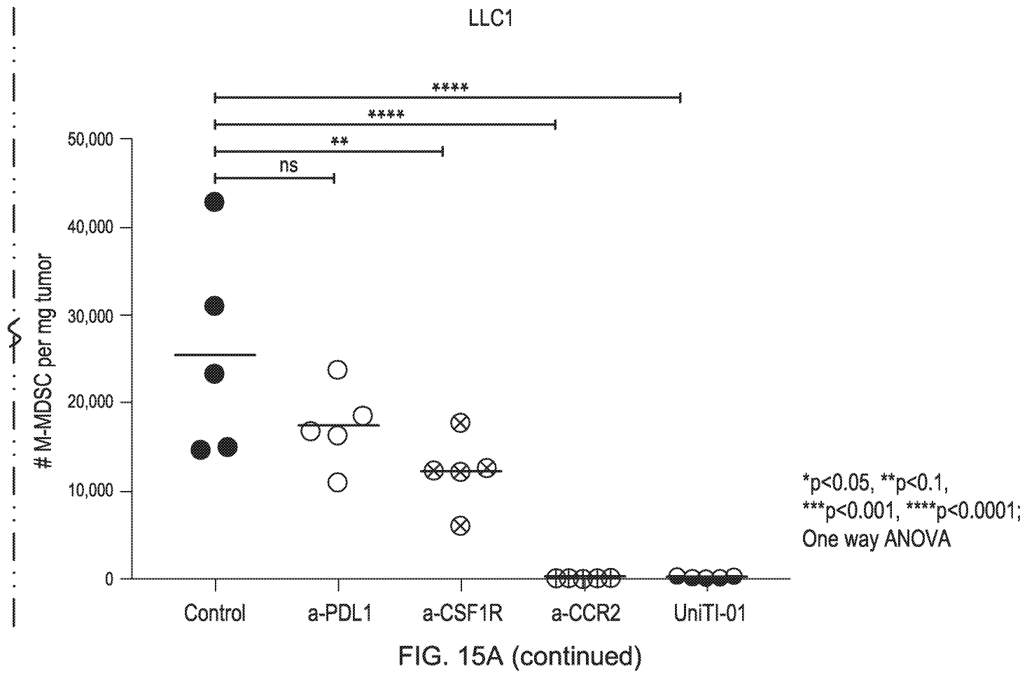

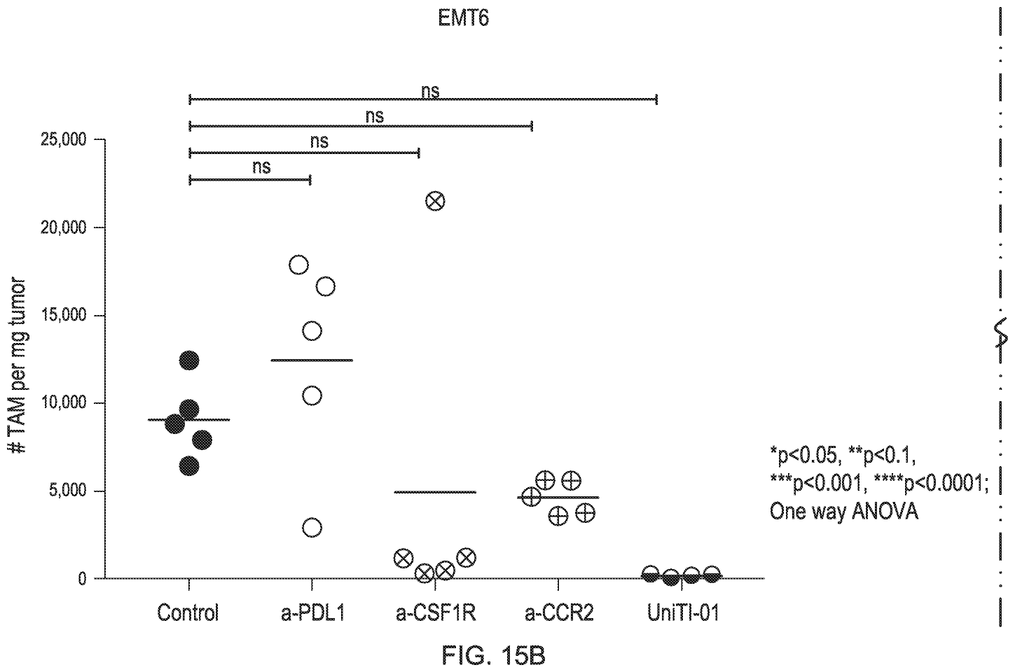

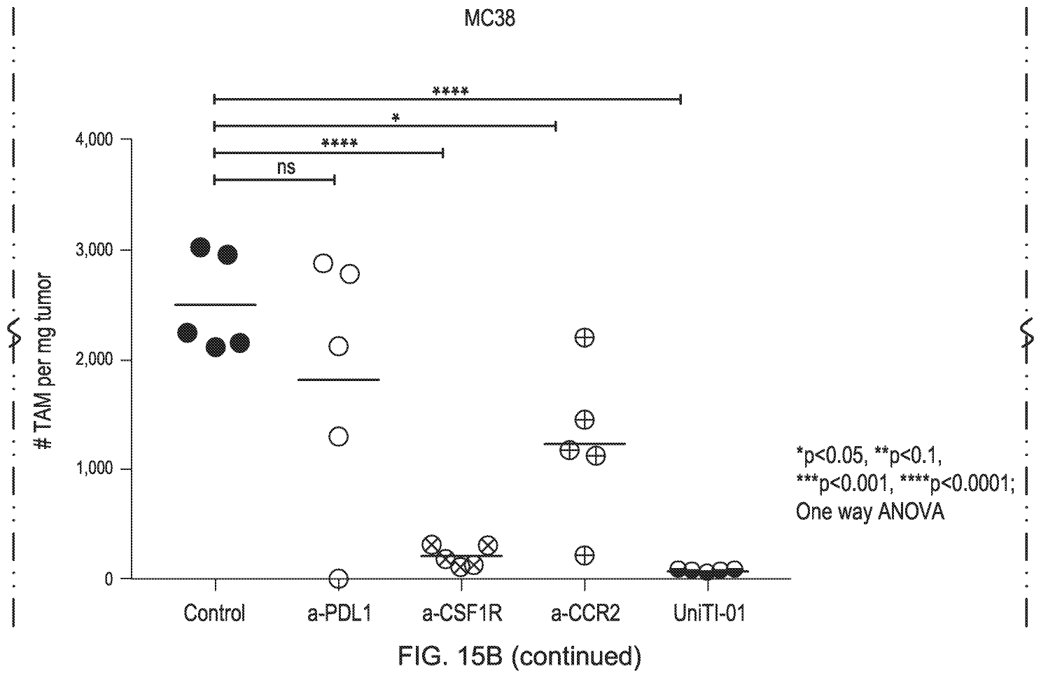

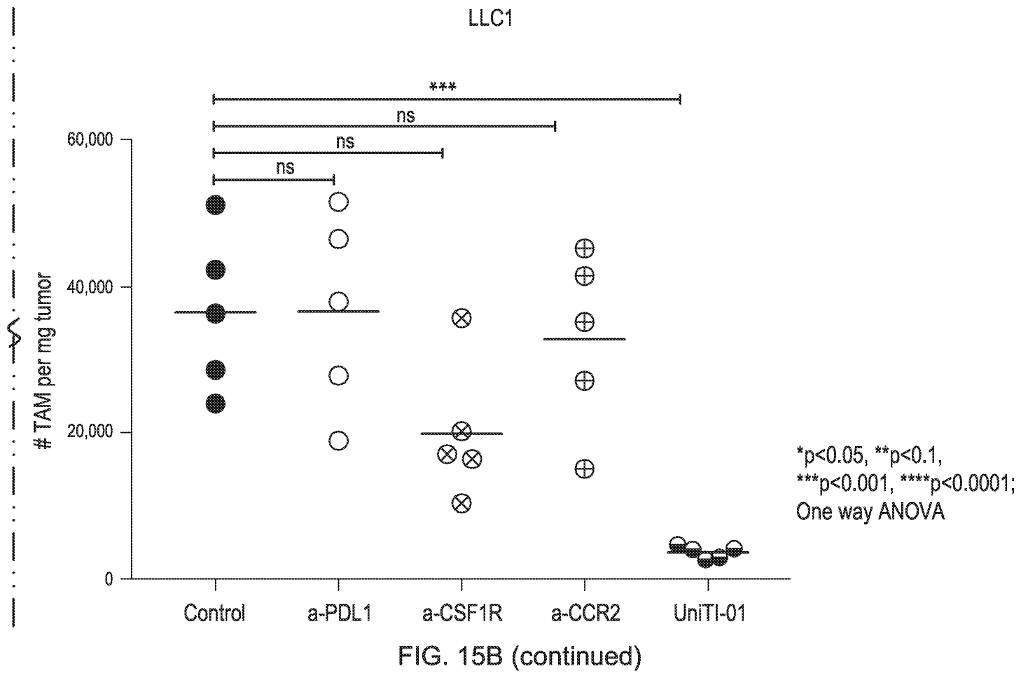

In this study, mice bearing EMT6, MC38, or LLC1 tumors received an anti-PDL1 bivalent monospecific antibody, an anti-CSF1R bivalent monospecific antibody, an anti-CCR2 bivalent monospecific antibody, or UniTI-01. The amount of M-MDSCs (FIG. 15A) or TAMs (FIG. 15B) per mg tumor was measured using flow cytometry analysis. As shown in FIGS. 15A and 15B, UniTI-01 drives depletion of M-MDSC and TAMs in EMT6, MC38, and LLC1 tumors.

### Example 15. UniTI-01 Preferentially Binds to Macrophages within Tumor Rather than Healthy Tissues

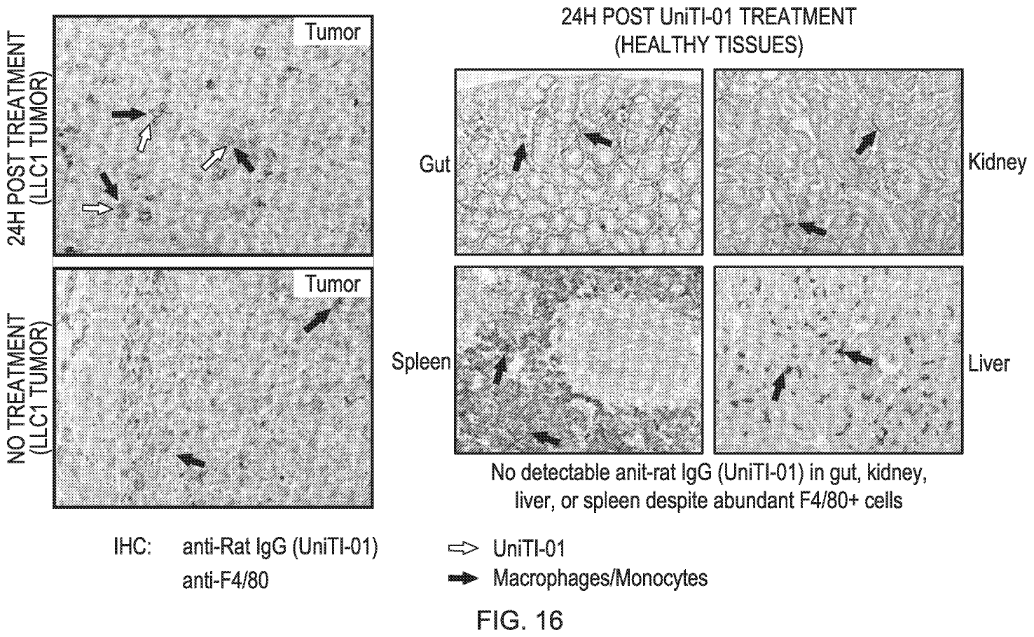

In this study, mice bearing LLC1 tumors received UniTI-01 treatment. 24 hours post UniTI-01 treatment, tissues from tumor, gut, kidney, spleen, or liver were analyzed by immunohistochemistry using an anti-rat IgG antibody (for detecting UniTI-01) and an anti-F4/80 antibody (for detecting macrophages and monocytes). As shown in FIG. 16, UniTI-01 administered to tumor bearing mice preferentially binds to macrophages within tumor. There was no detectable anti-rat IgG antibody staining in gut, kidney, liver, or spleen despite abundant F4/80+ cells.

### Example 16. Biodistribution of UniTI-01 in EMT6 Tumor Implanted Mice

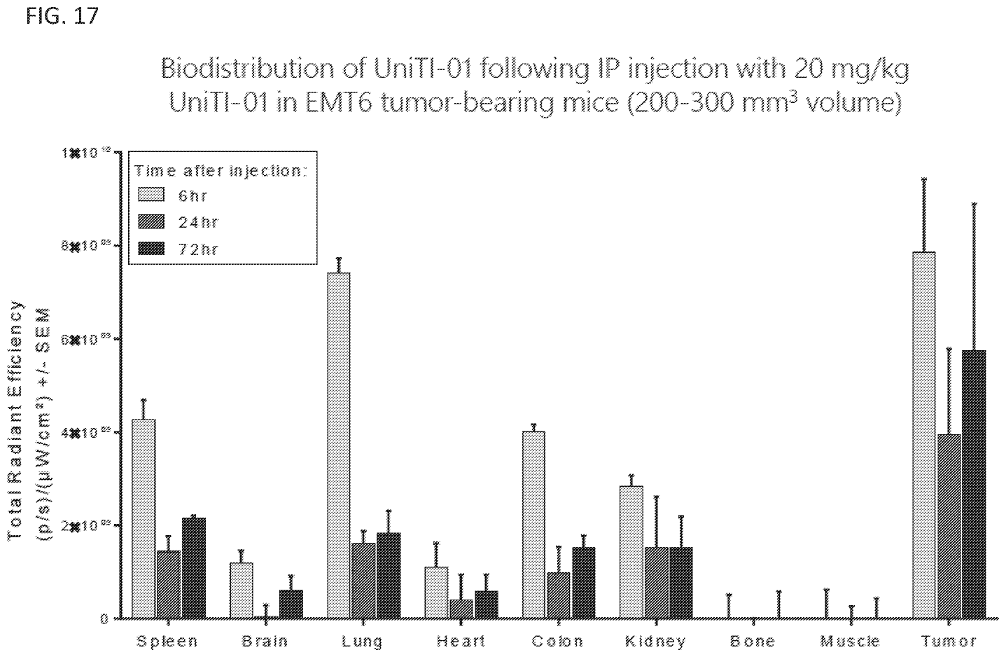

VivoTag 800 Fluorochrome labeled UniTI-01 was administered in EMT6 tumor implanted mice and detected by fluorescence imaging (signal normalized to untreated organ fluorescence). The biodistribution of UniTI-01 6-hour, 24-hour, or 72-hour after administration is shown in FIG. 17.

### Example 17. CSF-1R and CCR2 Expression on M-MDSC Cells in Ovarian Cancer Patients

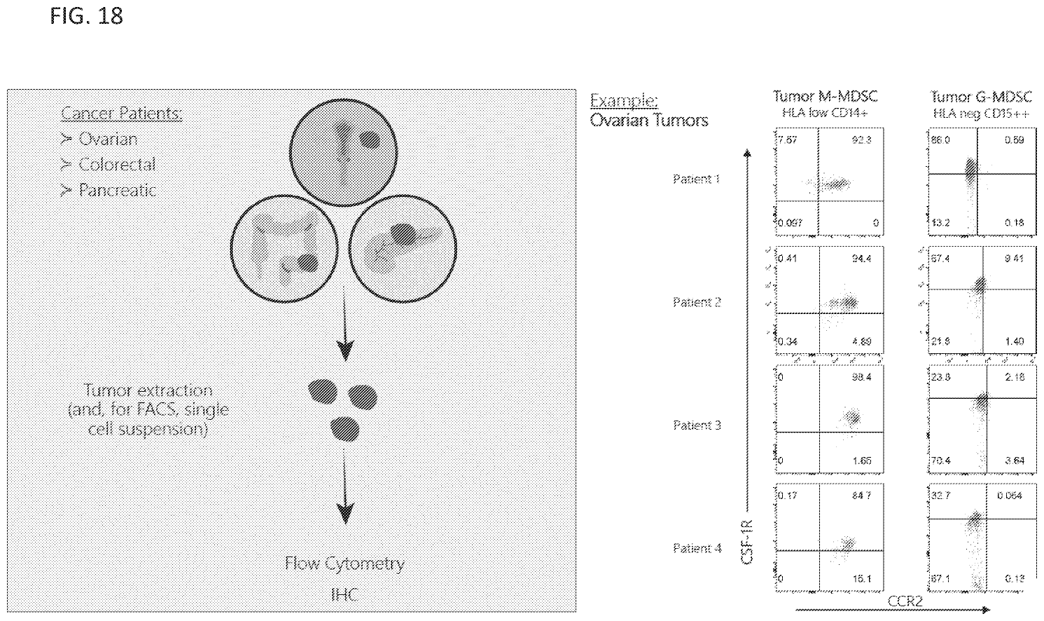

Tumor tissues were extracted from four ovarian tumor patients and analyzed for CSF1R and CCR2 expression. As shown in FIG. 18, tumor M-MDSCs show co-staining of CSF1R and CCR2, whereas tumor G-MDSCs do not. CSF-1R and CCR2 co-expression was also detected on CD163+ TAMs (data not shown).

### Example 18. UniTI-01 Increases T Cell Infiltration in Tumors

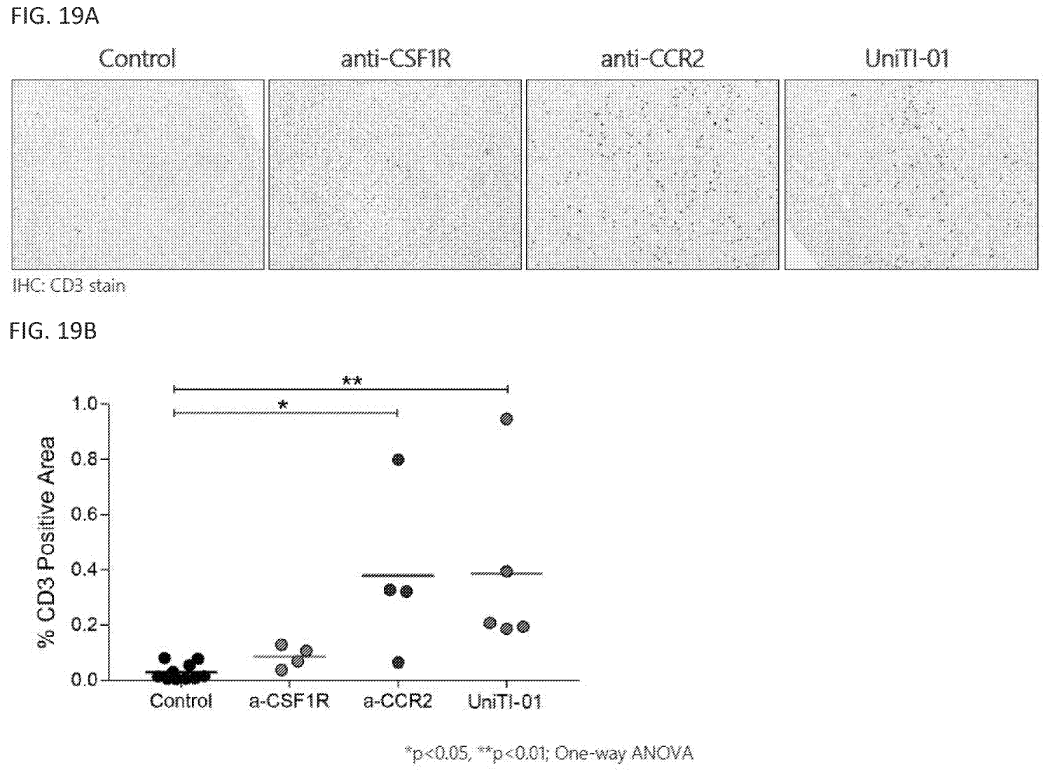

In this study, mice bearing EMT6 tumors received an anti-CSF1R bivalent monospecific antibody, an anti-CCR2 bivalent monospecific antibody, or UniTI-01. Tumor tissues were stained using an anti-CD3 antibody and % CD3 positive area was quantified. As shown in FIGS. 19A and 19B, compared with the anti-CSF1R bivalent monospecific antibody, UniTI-01 increases T cell infiltration in EMT6 tumors.

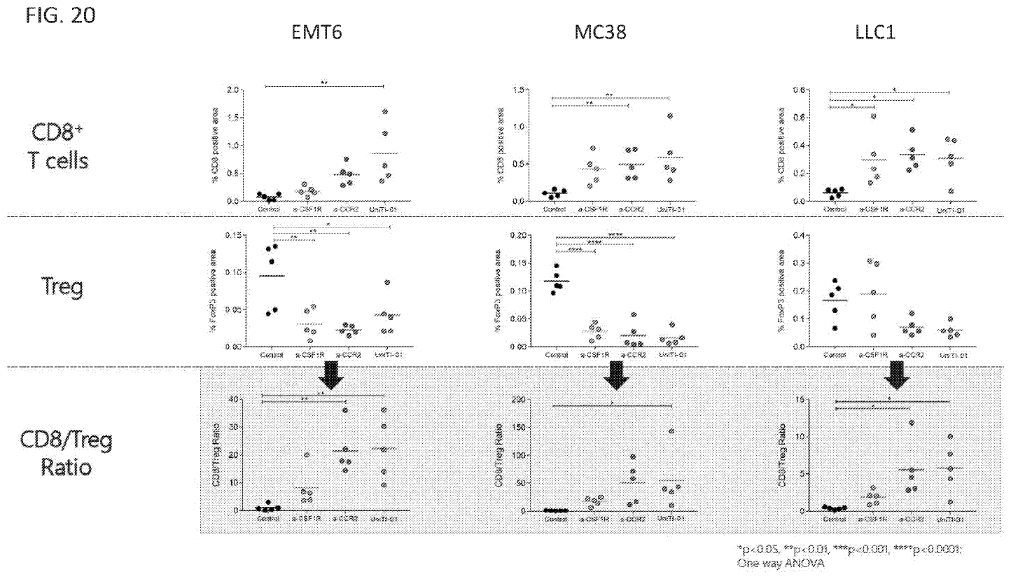

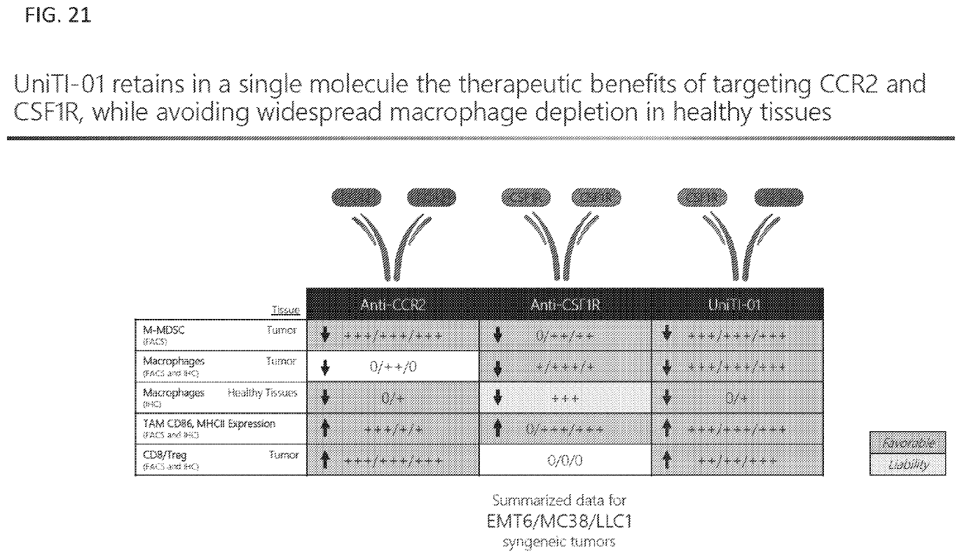

Additional immunohistochemistry analyses reveal that UniTI-01 increases CD8+ T cell/Treg ratio in EMT6, MC38 and LLC1 tumors (FIG. 20).

### Example 19. Additional In Vivo Studies Testing UniTI-01 in Combination with Anti-PDL1 Antibody

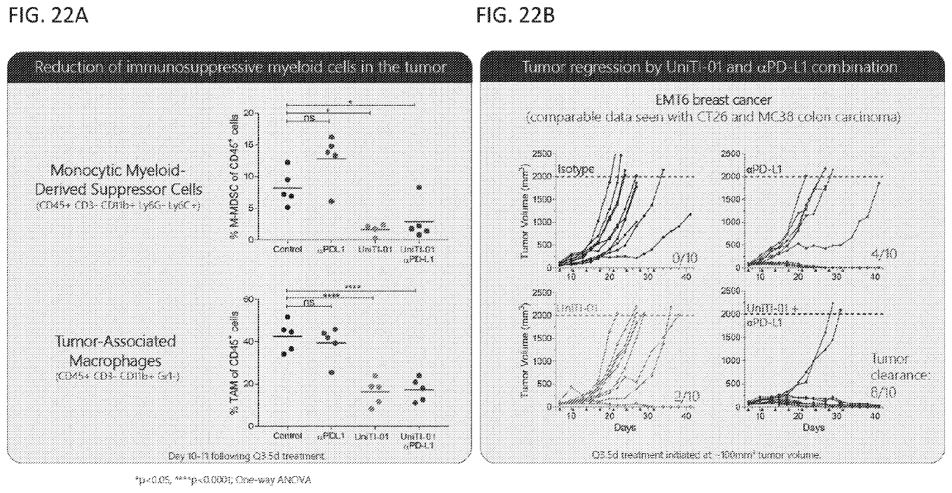

A number of in vivo studies were conducted to analyze the effect of UniTI-01 used in combination with an anti-PDL1 antibody. Compared with using the anti-PDL1 antibody as a single agent, combining UniTI-01 and the anti-PDL1 antibody reduces the amount of monocytic myeloid-derived suppressor cells and tumor-associated macrophages (FIG. 22A).

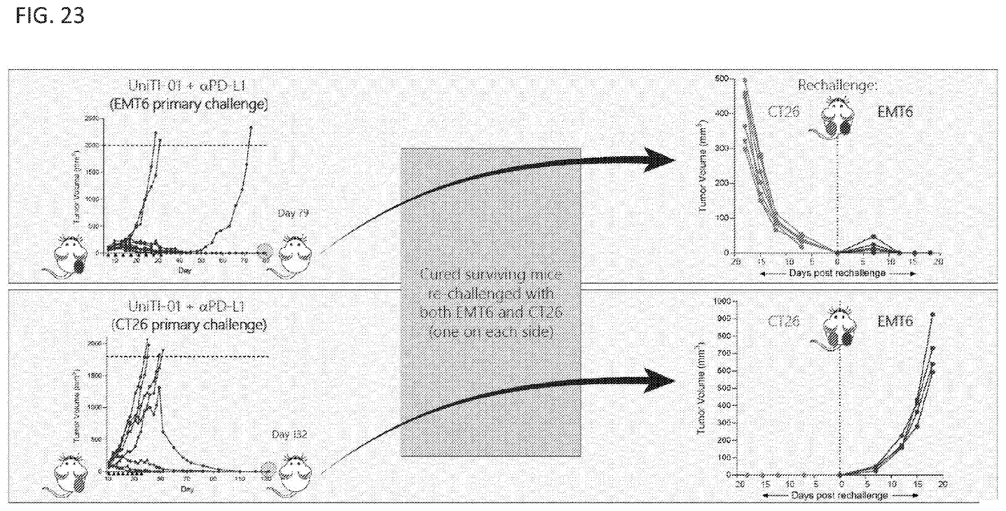

UniTI-01 and anti-PDL1 combination therapy shows not only durable anti-tumor responses (FIG. 23 left panel), but also development of immune memory against the original tumor (FIG. 23, right panel).

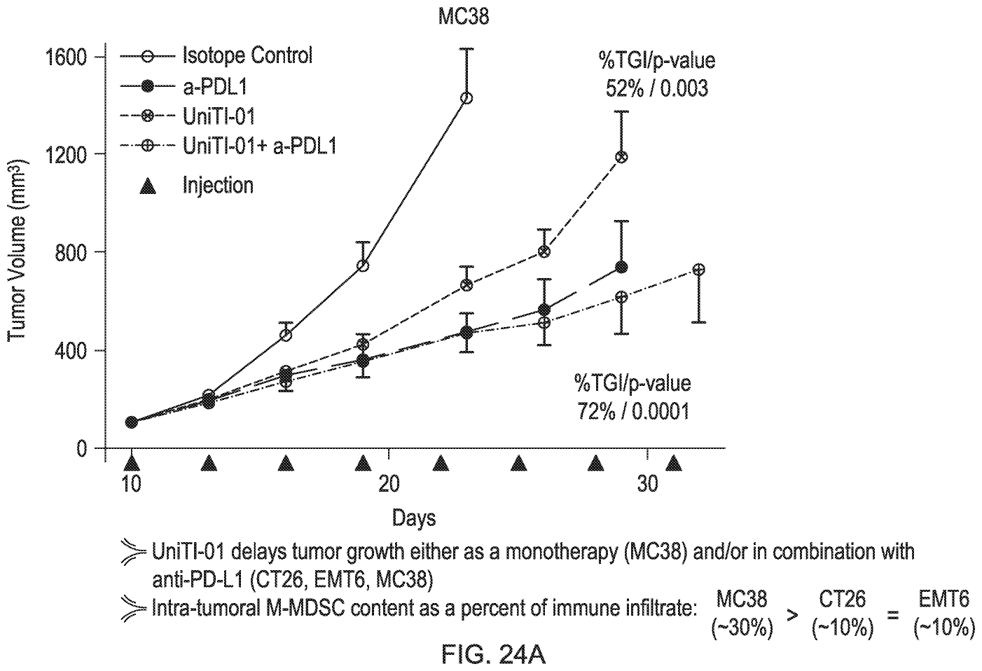

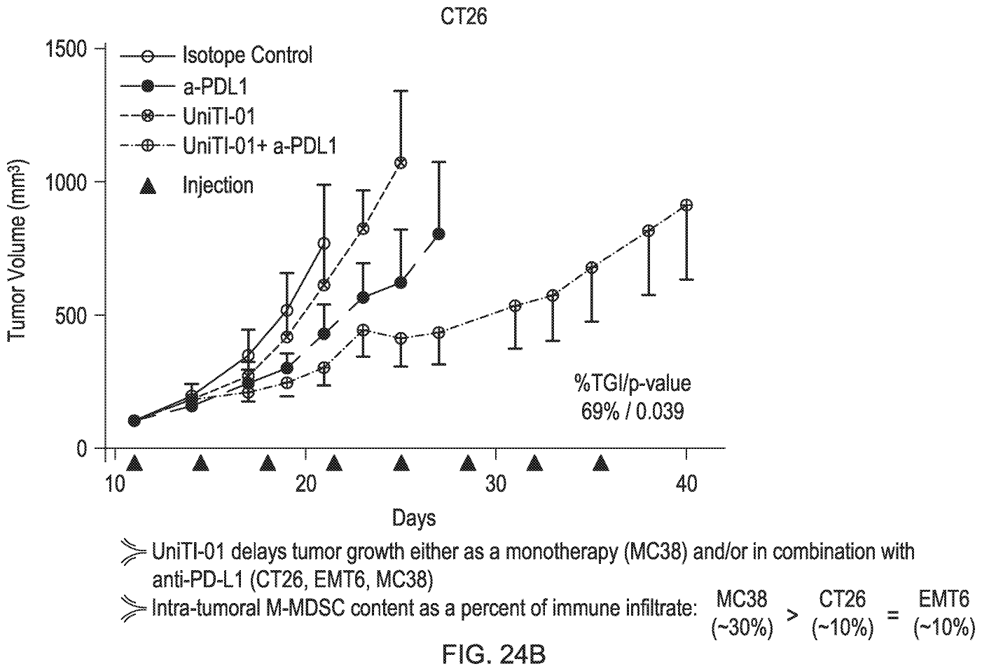

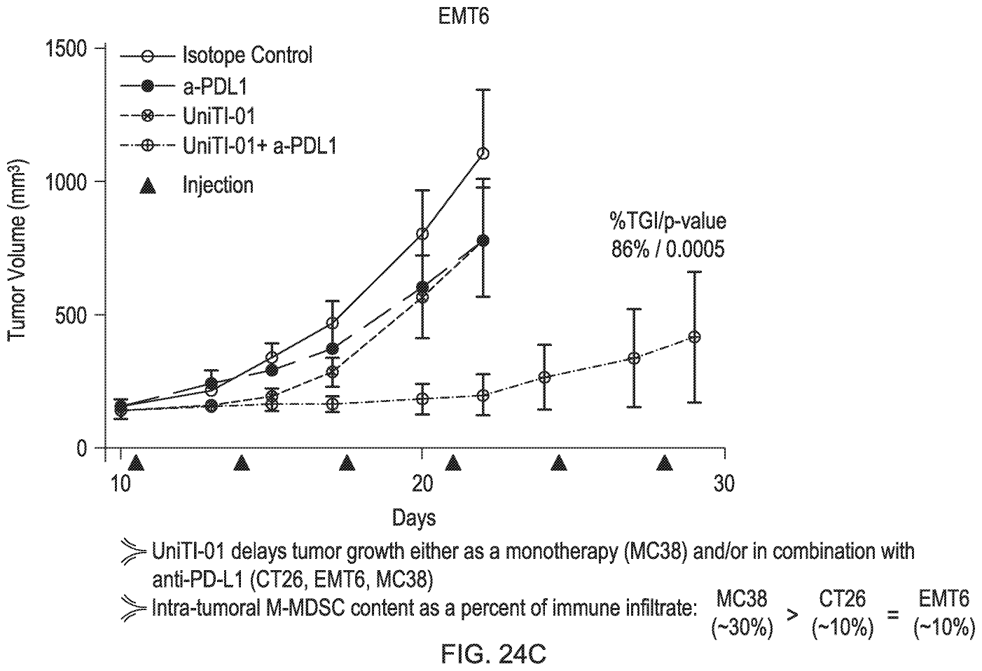

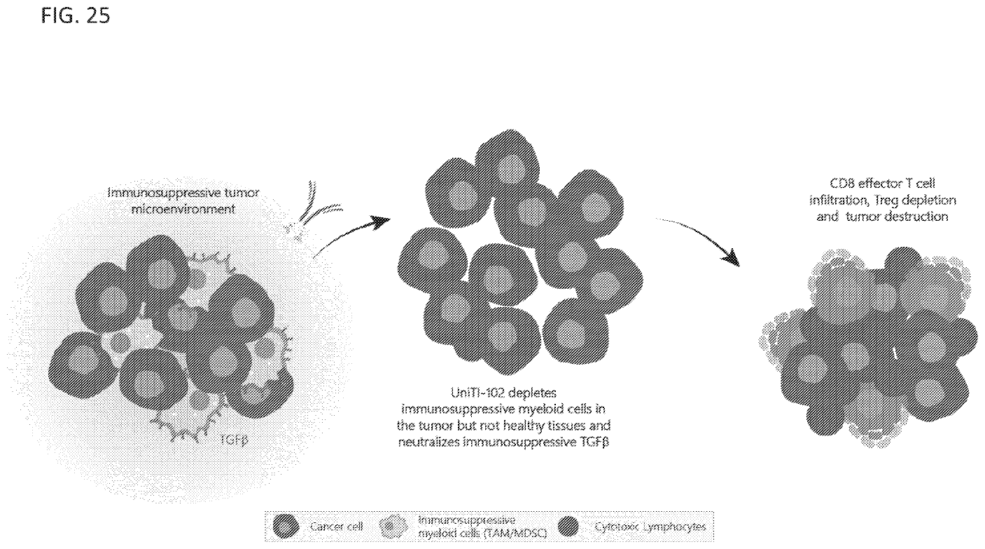

UniTI-01 shows single-agent efficacy in the MC38 syngeneic mouse model (FIG. 24A), whereas mice bearing CT26 or EMT6 tumors benefit from a combination therapy of UniTI-01 and anti-PDL1 antibody (FIGS. 24B and 24C). Without wishing to be bound by theory, the different anti-tumor activities of UniTI-01 in different tumor models may be, at least partially, due to the amount of intra-tumoral M-MDSC content. The intra-tumoral M-MDSC content as a percent of immune infiltrate for MC38 model, CT26 model, and EMT6 mole was around 30%, 10%, and 10%, respectively.

### Example 20. Characterization of UniTI-102

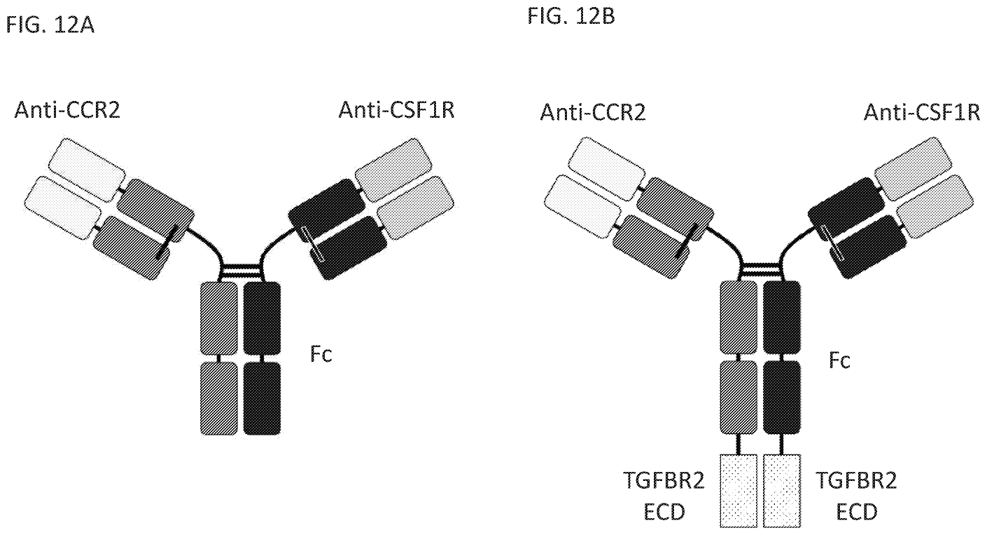

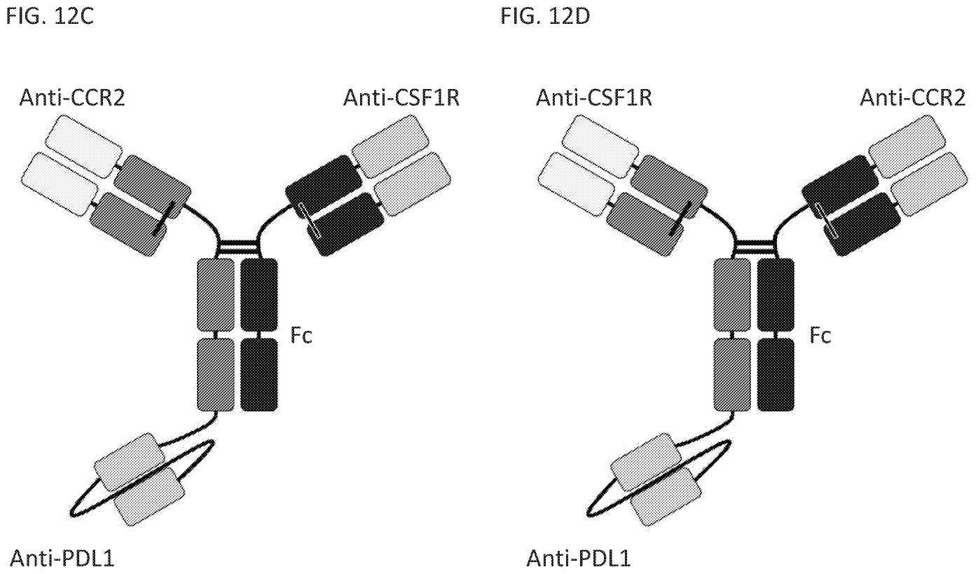

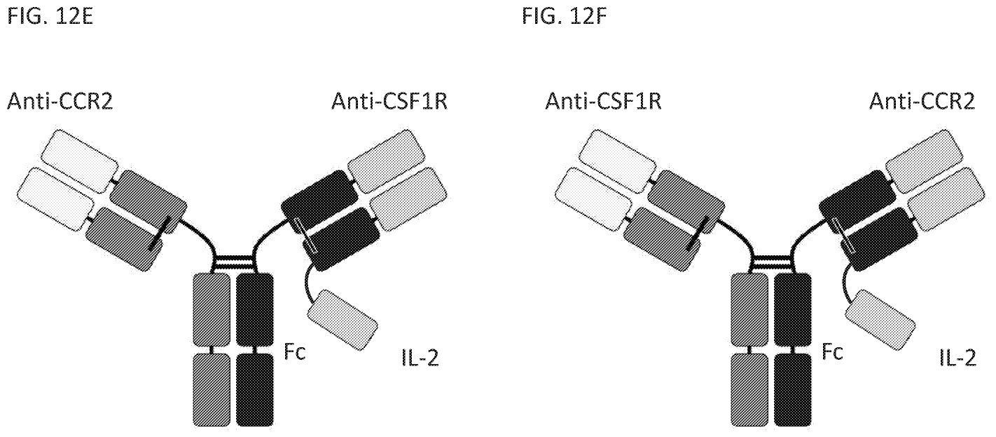

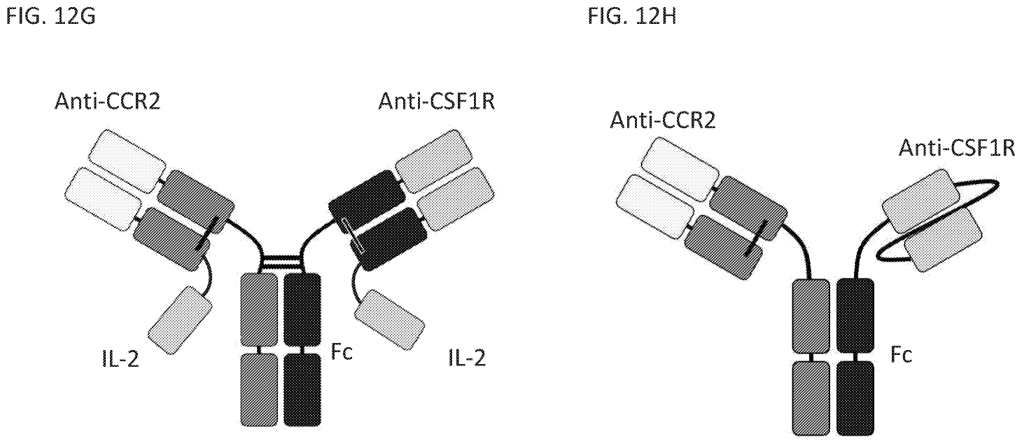

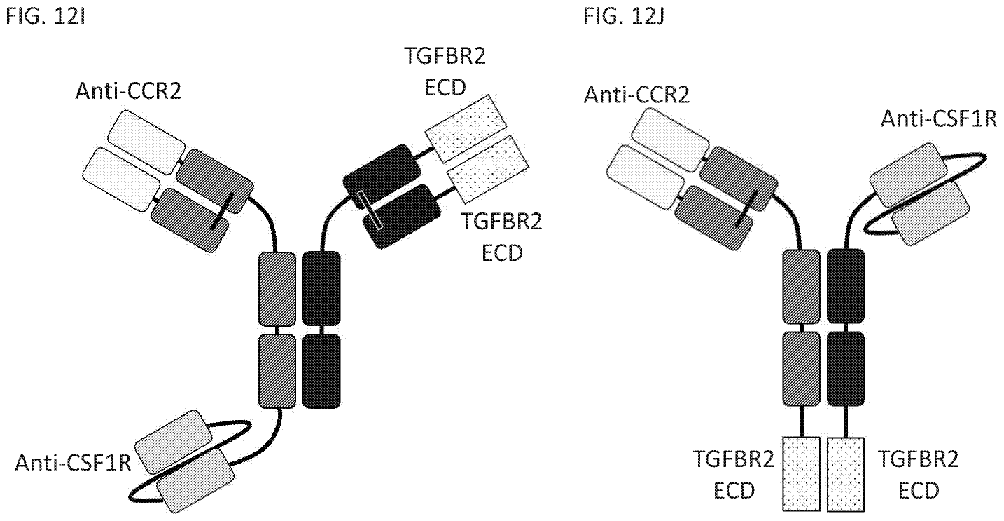

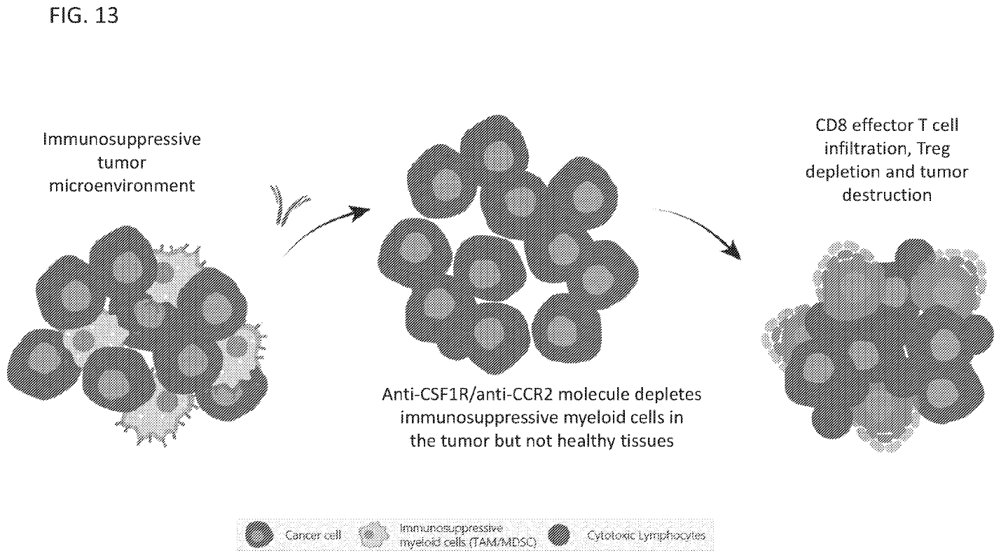

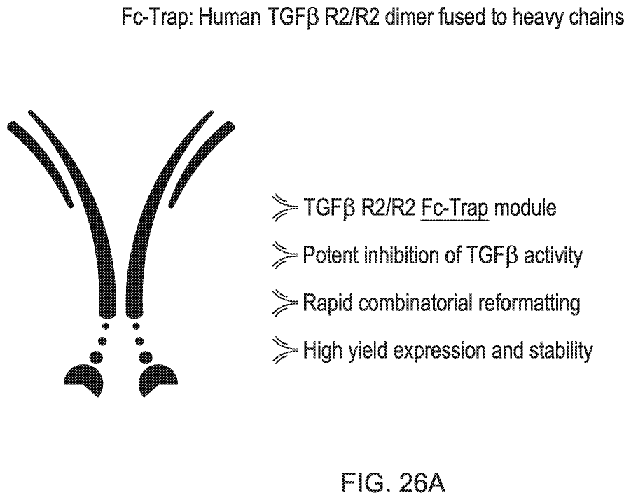

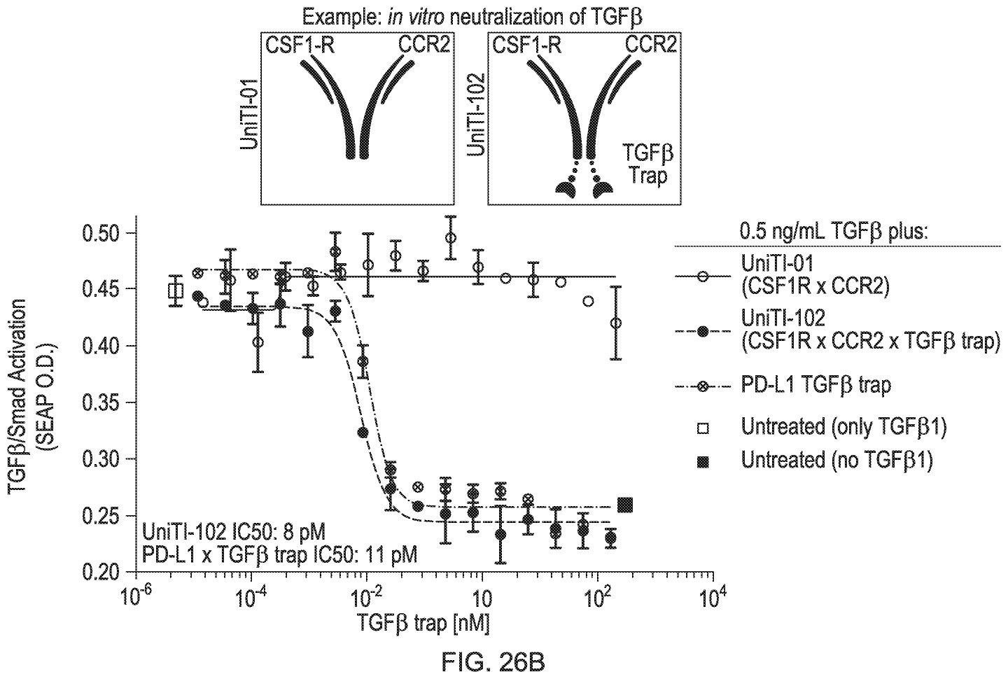

UniTI-01 was modified by fusing a human TGFβ R2/R2 dimer to the C-terminus of the heavy chains (FIG. 12B and FIG. 26A). This modified construct is named UniTI-102. First, the ability of UniTI-102 to neutralize TGFβ was tested. As shown in FIG. 26B, UniTI-102 effectively neutralizes TGFβ/Smad activation, whereas UniTI-01 does not.

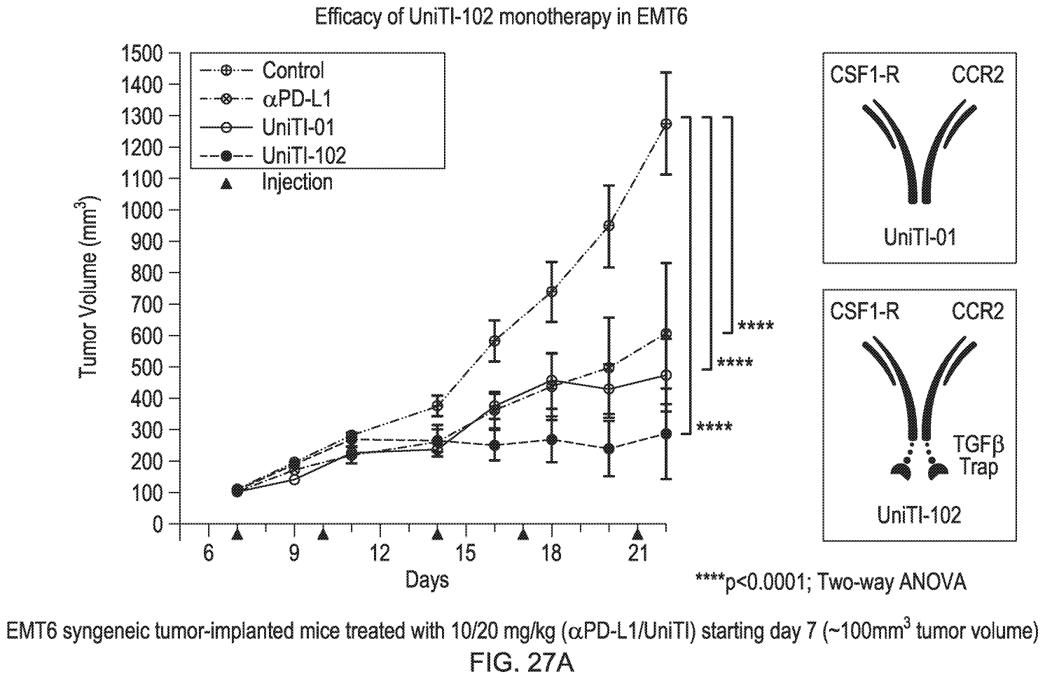

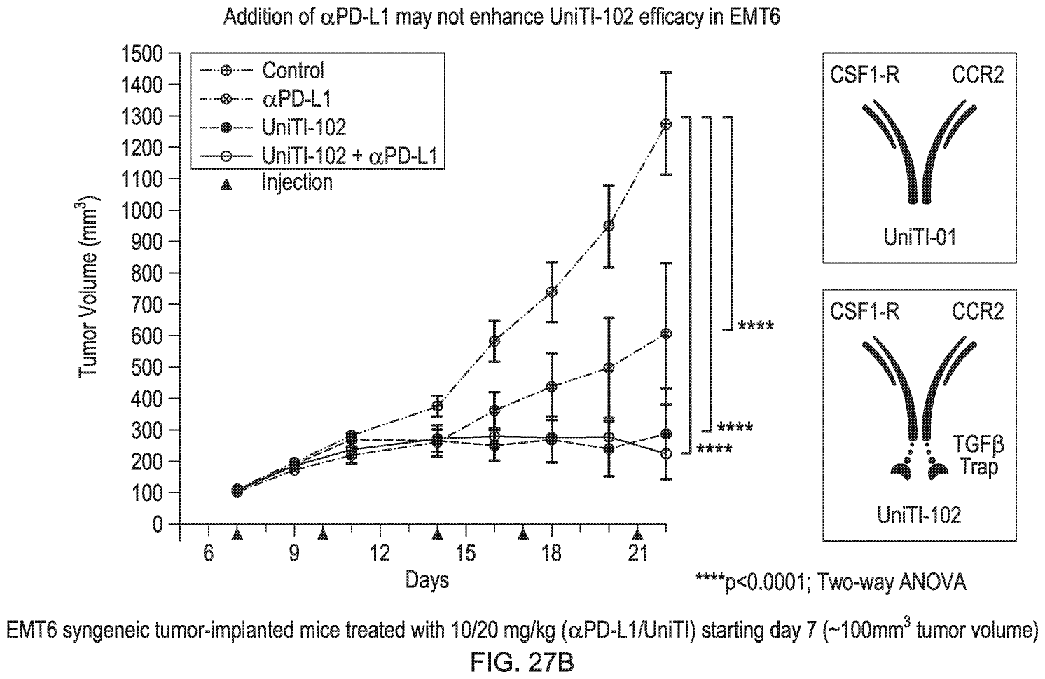

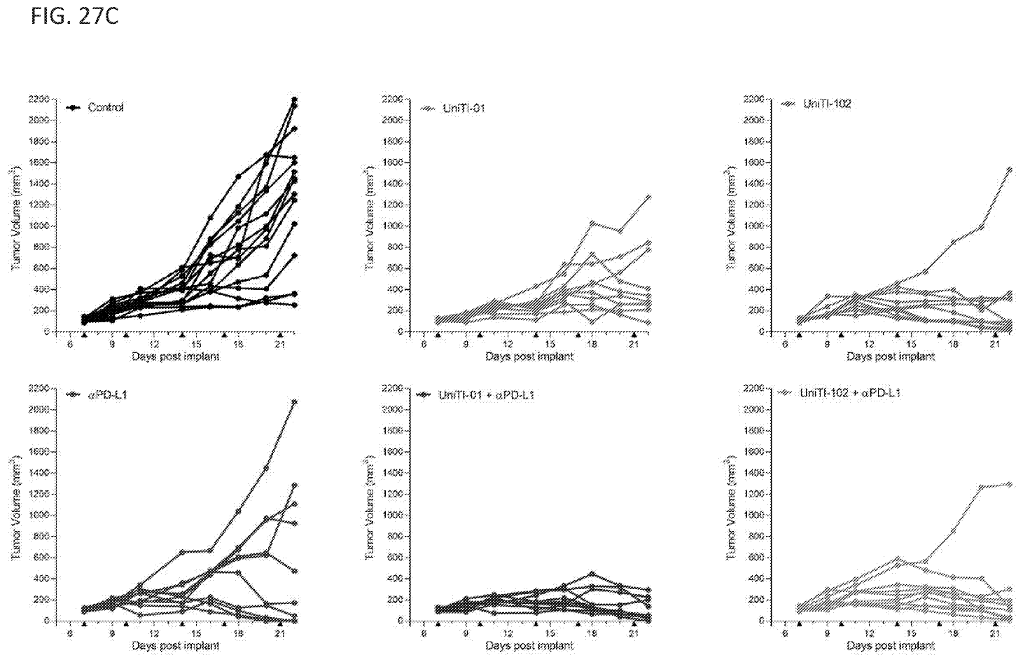

Next, mice implanted with EMT6 syngeneic tumor were administered with UniTI-01, UniTI-102, anti-PDL1, or a combination of UniTI-102 and anti-PDL1. As shown in FIGS. 27A, 27B, and 27C, UniTI-102 shows strong monotherapy response in EMT6 tumor model and is not enhanced with the addition of anti-PD-L1 under the condition tested.

### Example 21. Purification of Anti-CSF1R Half-Arm Fabs

Two human anti-human CSF1R antibodies B1117 and BI123 were optimized using yeast display. The antibody BI117 comprises a heavy chain variable region (VH) comprising the amino acid sequence of SEQ ID NO: 323 and a light chain variable region (VL) comprising the amino acid sequence of SEQ ID NO: 341, as disclosed in Table 15. The antibody BI123 comprises a VH comprising the amino acid sequence of SEQ ID NO: 337 and a VL comprising the amino acid sequence of SEQ ID NO: 342, as disclosed in Table 15. Affinity maturation using yeast display introduced modifications into the HCDR2 and HCDR3 of BI117, generating variants of the VH of BI117 (SEQ ID NOs: 324-336 shown in Table 15). Similarly, the HCDR2 and HCDR3 of BI123 were also mutated, leading to variants of the VH of BI123 (SEQ ID NOs: 338-340 shown in Table 15).

To analyze these affinity matured variants, half-arm antibodies BIM0542, BIM0543, BIM0544 BIM0545, BIM0546, BIM0547, BIM0548, BIM0549, BIM0550, BIM0551, BIM0552, BIM0553, BIM0554, BIM0555, BIM0556, BIM0566, and BIM0567 were expressed. The full-length sequence information of these antibodies is disclosed in Table 28. Briefly, all of these so-called half-arm antibodies comprise three chains: a heavy chain comprising a VH fused to a CH1 and an Fc comprising a hole (designated as HC 2 in Table 28), a cognate light chain (designated as LC 2 in Table 28), and a truncated his-tagged Fc domain comprising a knob (designated as HC 1 in Table 28). The VH/VL pairs contained in these half-arm antibodies are disclosed in Table 30.

TABLE 30
VH/VL pairs in half-arm antibodies.

| BIM ID | VH | VH SEQ ID NO | VL | VL SEQ ID NO |
|---|---|---|---|---|
| BIM0542 | aCSF1R BI117 | SEQ ID NO: 324 | aCSF1R BI117 | SEQ ID NO: 341 |
| | C5-R2A VH | | VL | |
| BIM0543 | aCSF1R BI117 | SEQ ID NO: 325 | aCSF1R BI117 | SEQ ID NO: 341 |
| | C9-R2A VH | | VL | |
| BIM0544 | aCSF1R BI117 | SEQ ID NO: 326 | aCSF1R BI117 | SEQ ID NO: 341 |
| | D1-R2A VH | | VL | |
| BIM0545 | aCSF1R BI117 | SEQ ID NO: 327 | aCSF1R BI117 | SEQ ID NO: 341 |
| | E2-R2B VH | | VL | |
| BIM0546 | aCSF1R BI117 | SEQ ID NO: 328 | aCSF1R BI117 | SEQ ID NO: 341 |
| | G1-R2B VH | | VL | |
| BIM0547 | aCSF1R BI117 | SEQ ID NO: 329 | aCSF1R BI117 | SEQ ID NO: 341 |
| | G12-R2B VH | | VL | |
| BIM0548 | aCSF1R BI117 | SEQ ID NO: 330 | aCSF1R BI117 | SEQ ID NO: 341 |
| | D6-R3B VH | | VL | |
| BIM0549 | aCSF1R BI117 | SEQ ID NO: 331 | aCSF1R BI117 | SEQ ID NO: 341 |
| | A6-R3B VH | | VL | |
| BIM0550 | aCSF1R BI117 | SEQ ID NO: 332 | aCSF1R BI117 | SEQ ID NO: 341 |
| | A11-R3B VH | | VL | |
| BIM0551 | aCSF1R BI117 | SEQ ID NO: 333 | aCSF1R BI117 | SEQ ID NO: 341 |
| | B2-R3B VH | | VL | |
| BIM0552 | aCSF1R BI117 | SEQ ID NO: 323 | aCSF1R BI117 | SEQ ID NO: 341 |
| | VH | | VL | |
| BIM0553 | aCSF1R BI117 | SEQ ID NO: 334 | aCSF1R BI117 | SEQ ID NO: 341 |
| | C5-R3B VH | | VL | |
| BIM0554 | aCSF1R BI123 | SEQ ID NO: 338 | aCSF1R BI123 | SEQ ID NO: 342 |
| | A5-R3B VH | | VL | |
| BIM0555 | aCSF1R BI123 | SEQ ID NO: 339 | aCSF1R BI123 | SEQ ID NO: 342 |
| | B6-R3B VH | | VL | |
| BIM0556 | aCSF1R BI123 | SEQ ID NO: 340 | aCSF1R BI123 | SEQ ID NO: 342 |
| | C1-R3B VH | | VL | |
| BIM0566 | aCSF1R BI117 | SEQ ID NO: 335 | aCSF1R BI117 | SEQ ID NO: 341 |
| | C6-R3B VH | | VL | |
| BIM0567 | aCSF1R BI117 | SEQ ID NO: 336 | aCSF1R BI117 | SEQ ID NO: 341 |
| | D11-R3B VH | | VL | |

Proteins were expressed in ExpiCHO system (GE lift tech) according to the manufacturer's instructions. Briefly, DNA encoding for sequences as outlined in Table 28 was mixed equally and used to transfected ExpiCHO cells. 5 days after transfection, the cells were measured for density and viability. The cells were transferred to 50 mL conical vials and centrifuged for 20 minutes at 18,000×g. Each supernatant was filtered through a 0.22 μm membrane. 2.0 mL of equilibrated Protein A resin was added to each construct. The mixture was incubated for 2 hours at room temperature before loaded into a 1.5 cm column. The resin was washed with 3×10 CV of PBS. The protein was eluted in 5 CV of Protein A elution buffer (20 mM citrate, 100 mM sodium chloride, pH 2.76) and collected in a single 10.0 mL fraction for each sample. Each sample was neutralized to pH 6.5 with 1M sodium citrate.

The protein content of the elution fraction was estimated using absorbance at 280 nm with the nanodrop. 1.0 mL of equilibrated Ni IMAC resin was added to each construct. The mixture was incubated for 2 hours at room temperature before loaded into a 1.5 cm column. The resin was washed with 3×10 CV of 50 mM Tris-HCl, 150 mM sodium chloride, pH 7.5. The protein was eluted in 5 CV of Ni IMAC elution buffer (50 mM Tris-HCl, 150 mM sodium chloride, 500 mM Imidazole, pH 7.5) and collected in a single 5.0 mL fraction. Each elution fraction was buffer exchanged into 20 mM citrate, 100 mM sodium chloride, pH 5.5 using PD-10 desalting columns. A gel (in MES buffer) of the load & flow-through (2 μL), elution and reduced elution fractions (5 μg) from the Protein A column was run to assess purity. The protein content of the elution fraction was estimated using absorbance at 280 nm with the nanodrop.

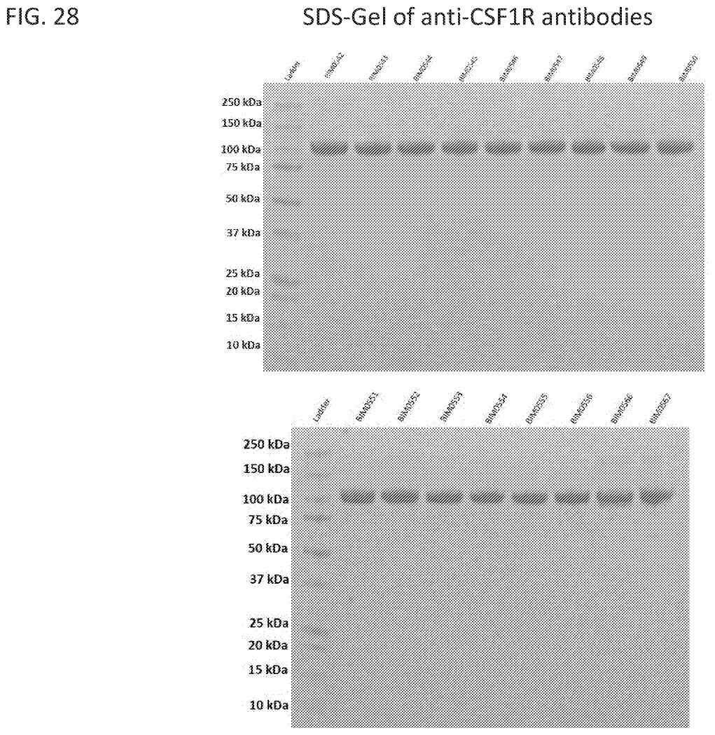

SDS-gel of the final purified antibodies is shown in FIG. 28 and yields are shown Table 31. In Table 31, the “mg/mL” column shows concentrations of the final purified samples, and the “mg/L” column shows an estimate of expression level per liter based on the culture volume (50 ml) used for the pilot experiment extrapolated to 1 L.

TABLE 31
final yield of affinity matured anti-human
CSF1R antibodies after purification

| Construct | A280 of 1 mg/mL | A280 | mg/mL | mL | mg | mg/L |
|---|---|---|---|---|---|---|
| BIM0542 | 1.47 | 0.70 | 0.48 | 7.0 | 3.36 | 67 |
| BIM0543 | 1.45 | 0.79 | 0.54 | 7.0 | 3.78 | 75 |
| BIM0544 | 1.47 | 0.77 | 0.52 | 7.0 | 3.64 | 72 |
| BIM0545 | 1.47 | 0.64 | 0.44 | 7.0 | 3.08 | 61 |
| BIM0546 | 1.45 | 0.74 | 0.51 | 7.0 | 3.57 | 71 |
| BIM0547 | 1.47 | 0.67 | 0.46 | 7.0 | 3.22 | 64 |
| BIM0548 | 1.47 | 0.77 | 0.52 | 7.0 | 3.64 | 72 |
| BIM0549 | 1.47 | 0.65 | 0.44 | 7.0 | 3.08 | 61 |
| BIM0550 | 1.47 | 0.64 | 0.44 | 7.0 | 3.08 | 61 |
| BIM0551 | 1.45 | 0.71 | 0.49 | 7.0 | 3.43 | 68 |
| BIM0552 | 1.47 | 0.54 | 0.37 | 7.0 | 2.59 | 51 |
| BIM0553 | 1.45 | 0.76 | 0.52 | 7.0 | 3.64 | 72 |
| BIM0554 | 1.58 | 0.40 | 0.25 | 7.0 | 1.75 | 35 |
| BIM0555 | 1.55 | 0.54 | 0.35 | 7.0 | 2.45 | 49 |
| BIM0556 | 1.53 | 0.69 | 0.45 | 7.0 | 3.15 | 63 |
| BIM0566 | 1.47 | 0.90 | 0.61 | 3.5 | 2.15 | 43 |
| BIM0567 | 1.47 | 1.38 | 0.94 | 3.5 | 3.29 | 65 |

### Example 22: ELISA Binding of Purified Anti-CSF1R Antibodies

Microplates were separately coated with 2 μg/mL of human or cyno CSF1R in 100 μL and blocked with 2% BSA. Serial dilutions of unlabeled monovalent human antibodies (11 points, 3-fold dilutions, 400 nM to 6.8 μM) were transferred to the coated and blocked plates at 75 μL/well and incubated for 1 hr at room temperature. Plates were washed three times and incubated for 30 mins with anti-human Fc horseradish peroxidase conjugate followed by addition of TMB, a substrate of HRP. The plates were developed for 5 mins, stopped with 1M HCL and read at a wavelength of 450 nm.

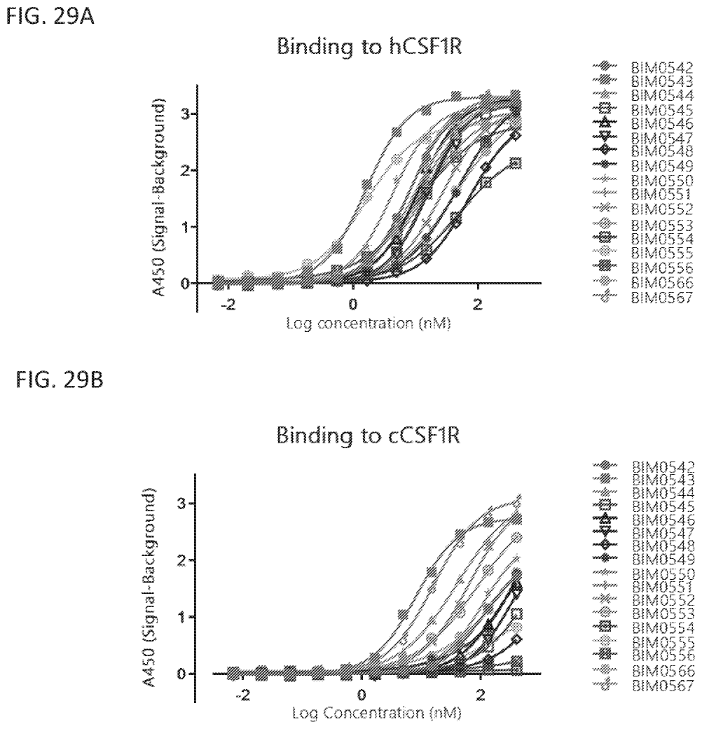

The BI117 and BI123 variants purified in Example 21 all showed binding to human CSF1R (FIG. 29A). The binding to cyno CSF1R varied between different constructs (FIG. 29B).

### Example 23: Functional Activity of Purified Anti-Human CSF1R Antibodies

Untouched CD14 positive human monocytes were purified using magnetic bead separation from negative selection of freshly isolated PBMCs. Monocytes were cultured at 37° C. in a 96-well plate in the presence of anti-CSF1R blocking antibodies and hCSF-1 for 24 hours. Cell culture supernatants were harvested for measuring CCL2 (MCP-1) secretion. Monocytes alone served as minimal MCP-1 secretion. Monocytes in the presence of hCSF-1 served as maximum MCP-1 secretion. MCP-1 was measured using electrochemiluminescence (Mesoscale Discovery).

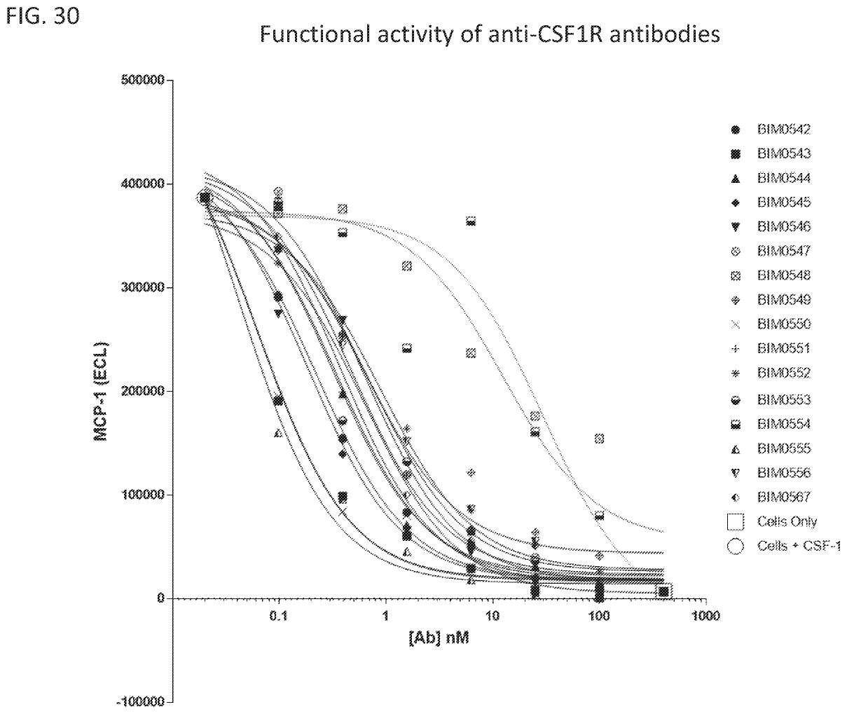

As shown in FIG. 30, the presence of monovalent CSF1R blocking antibodies led to concentration-dependent reduction of MCP-1. Anti-CSF1R monovalent constructs BIM0542, BIM0543, BIM0544, BIM0545, BIM0546, BIM0547, BIM0549, BIM0550, BIM0551, BIM0552, BIM0553, BIM0555, BIM0556, BIM0567 showed IC50s in the range of 0.045-0.9 nM (FIG. 30). BIM0548 and BIM0554 showed IC50s up to 30 nM (FIG. 30).

### Example 24: Cell Binding of Purified Anti-CSF1R Antibodies

HEK-293T cells were lentivirally transduced with human CSF1R and stable cell lines were generated. Nearly all transduced cells expressed high levels of CSF1R. Cells were washed with PBS containing 0.5% BSA and 0.1% sodium azide (staining buffer) and added to 96-well V-bottom plates with 100,000 cells/well. Human anti-CSF1R monovalent antibodies were added to the cells in 2.5 fold serial dilutions and incubated for 2 hour at room temperature. The plates were washed twice with staining buffer. The secondary antibody against human Fc conjugated to APC was added at 1:200 dilution (1.5 mg/ml stock) and incubated with the cells for 1 hour at 4° C. followed by washing with staining buffer. Cells were subsequently stained with a live/dead dye to exclude any dying cells and were fixed for 10 minutes with 4% paraformaldehyde at room temperature. The plates were read on CytoFLEX LS (Beckman Coulter). Data was calculated as the percent-APC positive population.

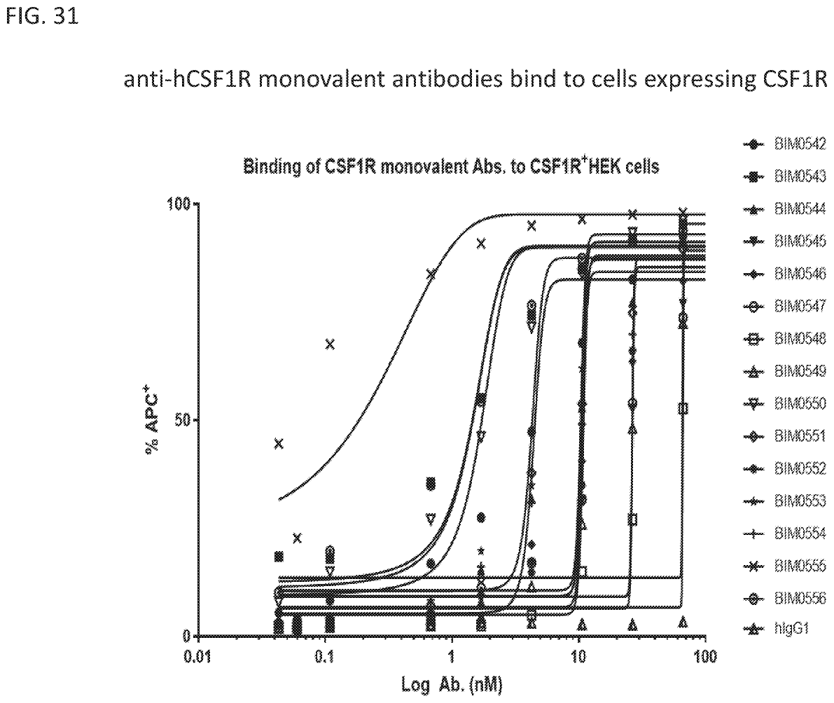

As shown in FIG. 31, all the anti-CSF1R monovalent antibodies tested showed binding the cells expressing human CSF1R.

### Example 25: Expression and Purification of Selected Bispecific CCR2×CSF1R Kappa/Lambda Antibodies

Anti-CCR2/anti-CSF1R (“CCR2×CSF1R”) bispecific antibodies BIM0204, BIM0205, BIM0206, BIM0207, BIM0208, BIM0209, BIM0210, and BIM0211 were generated as described below. The full-length sequence information of these antibodies is disclosed in Table 28. Briefly, these antibodies comprise an anti-CCR2 arm (designated as HCl and LC 1 in Table 28) and an anti-CSF1R arm (designated as HC 2 and LC 2 in Table 28). The VH/VL pairs contained in these bispecific antibodies are summarized in Table 32. The anti-CCR2 binding arm comprises a kappa light chain whereas the anti-CSF1R binding arm comprises a lambda light chain.

TABLE 32
VH/VL pairs in bispecific antibodies.

| | Anti-CCR2 arm | | | | Anti-CSF1R arm | | | |
|---|---|---|---|---|---|---|---|---|
| | | VH SEQ | | VL SEQ | | VH SEQ | | VL SEQ |
| BIM ID | VH | ID NO | VL | ID NO | VH | ID NO | VL | ID NO |
| BIM0204 | aCCR2 | SEQ ID | aCCR2 | SEQ ID | aCSF1R | SEQ ID | aCSF1R | SEQ ID |
| | Hum1 VH | NO: 343 | VL | NO: 345 | BI117 VH | NO: 323 | BI117 VL | NO: 341 |
| BIM0205 | aCCR2 | SEQ ID | aCCR2 | SEQ ID | aCSF1R | SEQ ID | aCSF1R | SEQ ID |
| | Hum1 VH | NO: 343 | VL | NO: 345 | BI123 VH | NO: 337 | BI123 VL | NO: 342 |
| BIM0206 | aCCR2 | SEQ ID | aCCR2 | SEQ ID | aCSF1R | SEQ ID | aCSF1R | SEQ ID |
| | Hum2 VH | NO: 344 | VL | NO: 345 | BI117 VH | NO: 323 | BI117 VL | NO: 341 |
| BIM0207 | aCCR2 | SEQ ID | aCCR2 | SEQ ID | aCSF1R | SEQ ID | aCSF1R | SEQ ID |
| | Hum2 VH | NO: 344 | VL | NO: 345 | BI123 VH | NO: 337 | BI123 VL | NO: 342 |
| BIM0208 | aCCR2 | SEQ ID | aCCR2 | SEQ ID | aCSF1R | SEQ ID | aCSF1R | SEQ ID |
| | Hum1 VH | NO: 343 | VL | NO: 345 | BI117 VH | NO: 323 | BI123 VL | NO: 342 |
| BIM0209 | aCCR2 | SEQ ID | aCCR2 | SEQ ID | aCSF1R | SEQ ID | aCSF1R | SEQ ID |
| | Hum1 VH | NO: 343 | VL | NO: 345 | BI123 VH | NO: 337 | BI117 VL | NO: 341 |
| BIM0210 | aCCR2 | SEQ ID | aCCR2 | SEQ ID | aCSF1R | SEQ ID | aCSF1R | SEQ ID |
| | Hum2 VH | NO: 344 | VL | NO: 345 | BI117 VH | NO: 323 | BI123 VL | NO: 342 |
| BIM0211 | aCCR2 | SEQ ID | aCCR2 | SEQ ID | aCSF1R | SEQ ID | aCSF1R | SEQ ID |
| | Hum2 VH | NO: 344 | VL | NO: 345 | BI123 VH | NO: 337 | BI117 VL | NO: 341 |

Prior to transfection, CHO cells (3E7 at a viability of >99%) were seeded at a density of ˜2×10⁶ cells/mL into 8×2L Erlenmeyer shake flasks containing 450 mL of CD Forti CHO and 2 mM L-glutamine. Co-transfection of all 4 chains was conducted. DNA-PEI mixture was added to each flask. Flasks were swirled and incubated at 37° C. with 5% CO₂ and 130 rpm. Temperature was shifted to 32° C. on day 2. 10% feed C, 2 mM L-glutamine, and 2 g/L glucose were added on day 2 and day 5 and 0.5 mM Sodium butyrate was added on day 5. 150 mL of fresh media, 5% feed C, 2 mM L-glutamine, and 2 g/L glucose was added to the batch on day 7 and the cells were harvested on day 9.

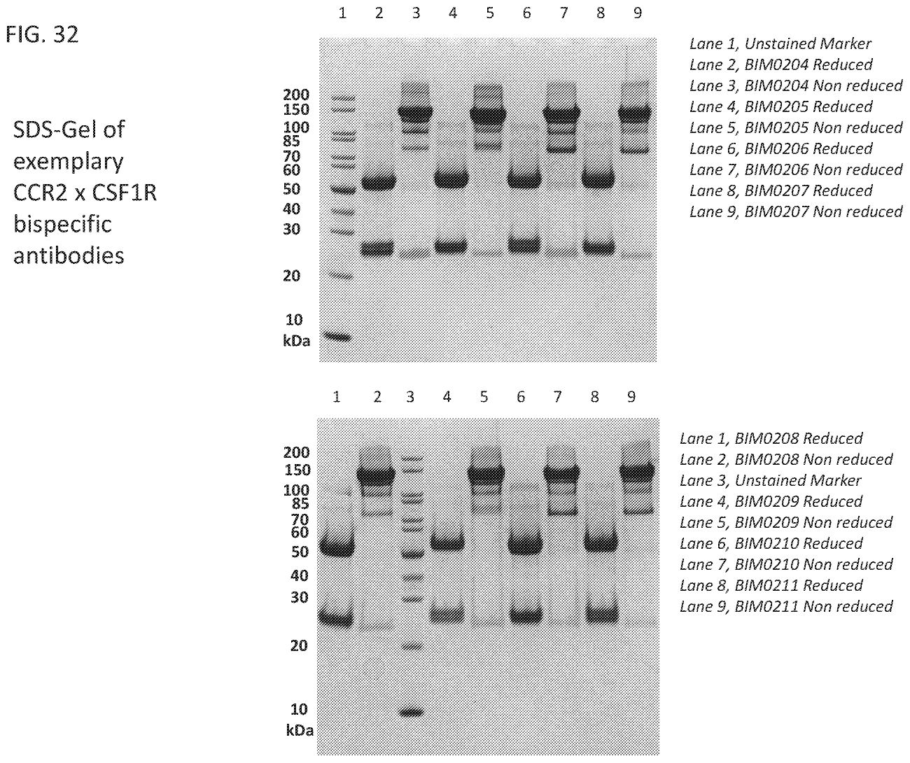

500-600 ml of the culture supernatant was incubated with 2 ml of MabSelectSure resin for ˜16 h at 4° C. on a rocking platform. The resin was collected and washed with 1×PBS. Protein was eluted using 30 mM Sodium Acetate pH 3.6, 100 mM NaCl in 4 fractions of 2 ml each. 10 μL of the fractions were checked on reduced or non-reduced SDS-PAGE. The fractions were pooled and pH adjusted to ˜pH 5.0 and concentration estimated using UV absorbance. SDS-PAGE of the final purified bispecific CCR2×CSF1R antibodies is shown in FIG. 32 and the final yield is shown in Table 33.

TABLE 33
Final yield of purified CCR2 x CSF1R
bispecific kappa/lambda antibodies

| Protein Name | Protein concentration mg/mL | Volume (mL) |
|---|---|---|
| BIM0204 | 5.1 | 8 |
| BIM0205 | 7.1 | 8 |
| BIM0206 | 5.4 | 8 |
| BIM0207 | 6.12 | 8 |
| BIM0208 | 5.8 | 8 |
| BIM0209 | 5.7 | 8 |
| BIM0210 | 7.4 | 8 |
| BIM0211 | 5.1 | 8 |

### Example 26: Functional Activity of Bispecific CCR2×CSF1R Antibodies

Tango-CCR2 bla2 U2OS cells (Invitrogen) express human CCR2, which in response to the ligand, CCL2, emit blue signal driven by beta-lactamase reporter gene (at 460 nm). The reporter cells were cultured in a 96-well plate in the presence of CCL2 for 16 hours at 37° C. before the addition of BLA substrate. In the absence of CCL2, the addition of substrate leads to green emission (at ˜530 nm).

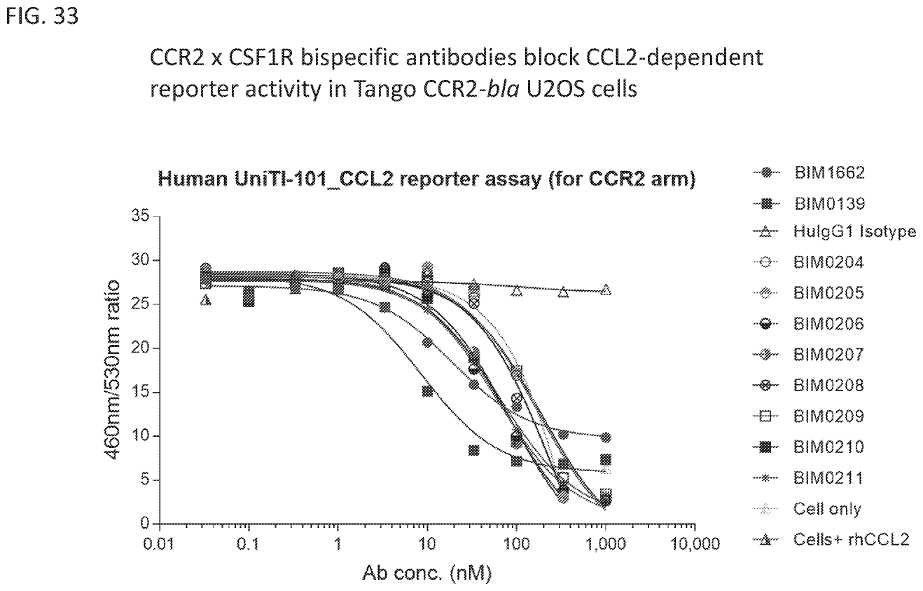

The presence of anti-CCR2 blocking antibodies led to concentration-dependent reduction in the emission at 460 nm (over 530 nM) (FIG. 33). The bivalent anti-CCR2 antibody positive controls, BHM1662 and BHM0139, showed IC50 at 19.1 nM and 8.5 nM, respectively. The human IgG1 showed no CCR2-dependent signal inhibition. Bispecific antibody constructs BIM0206, BHI0207, BIM0210 and BIM0211 showed IC50 in the range of 59-82 nM while IC50 of BIM0204, BIM0205, BIM0208 and BIM0209 ranged from 150-650 nM (FIG. 33).

Untouched CD14 positive human monocytes were purified using magnetic bead separation from negative selection of freshly isolated PBMCs. Monocytes were cultured at 37° C. in a 96-well plate in the presence of anti-CSF1R blocking antibodies and hCSF-1 for 24 hours. Cell culture supernatants were harvested for measuring CCL2 (MCP-1) secretion. Monocytes alone served as minimal MCP-1 secretion. Monocytes in the presence of hCSF-1 served as maximum MCP-1 secretion. MCP-1 was measured using electrochemiluminescence (Mesoscale Discovery).

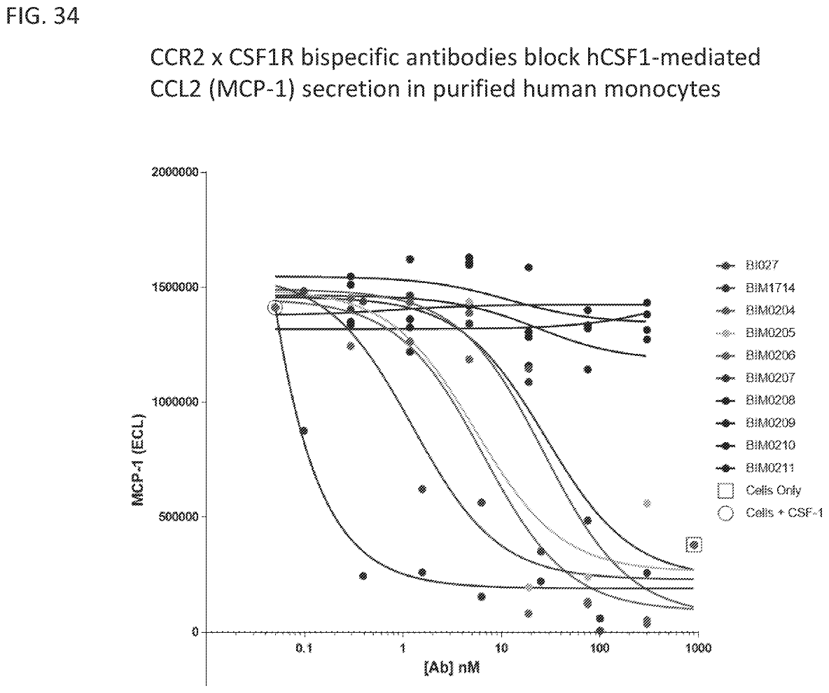

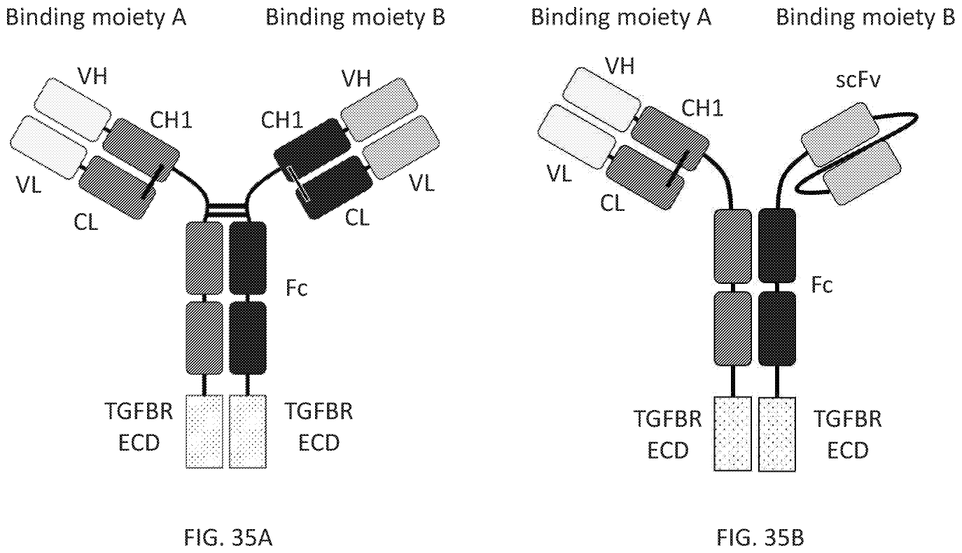

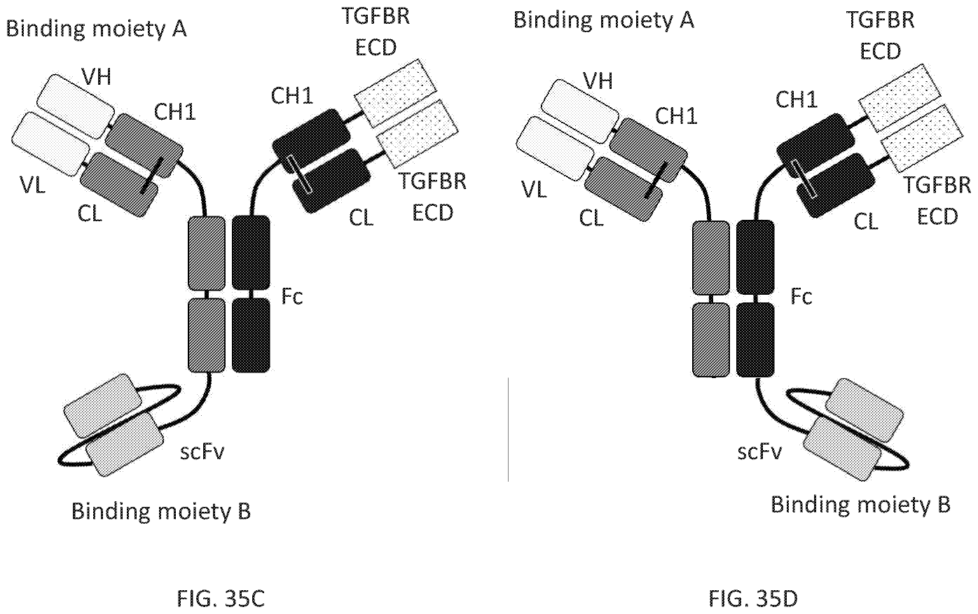

The presence of anti-CSF1R blocking antibodies led to concentration-dependent reduction of MCP-1 (FIG. 34). The monovalent and bivalent anti-CSF1R antibody positive controls, BI027 and BHM1714, showed IC50s of 1.25 nM and −0.002 nM, respectively (FIG. 34). Bispecific antibody constructs BIM0204, BIM0205, BIM0206, and BIM0207 showed IC50 in the range of 5-30 nM while there was no CSF1R blocking activity for BIM0208, BIM0209, BIM0210, and BIM0211 (FIG. 34).

### Example 27: Inhibition of TGFβ Signaling Using TGFβ Trap

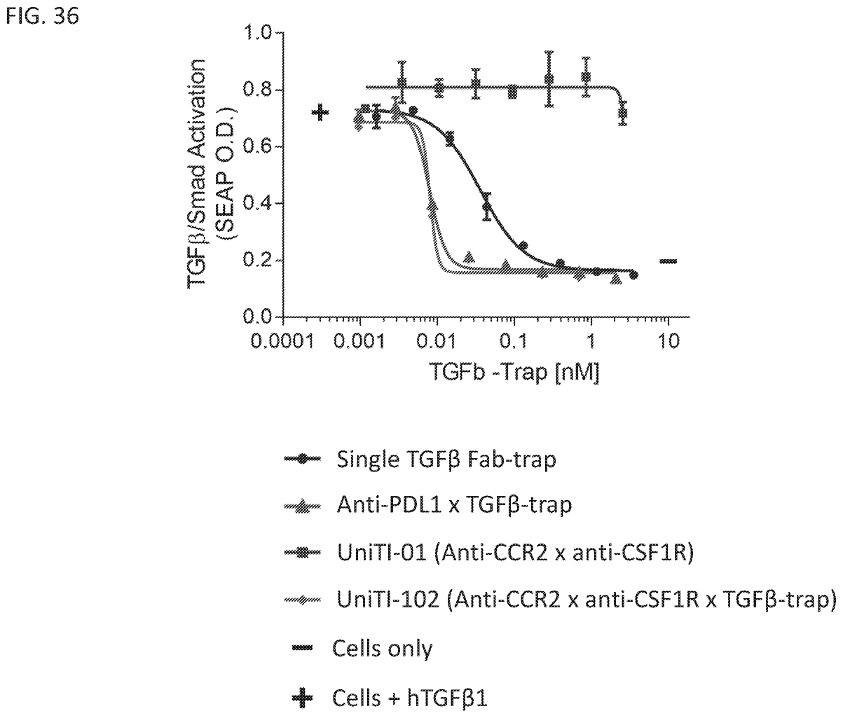

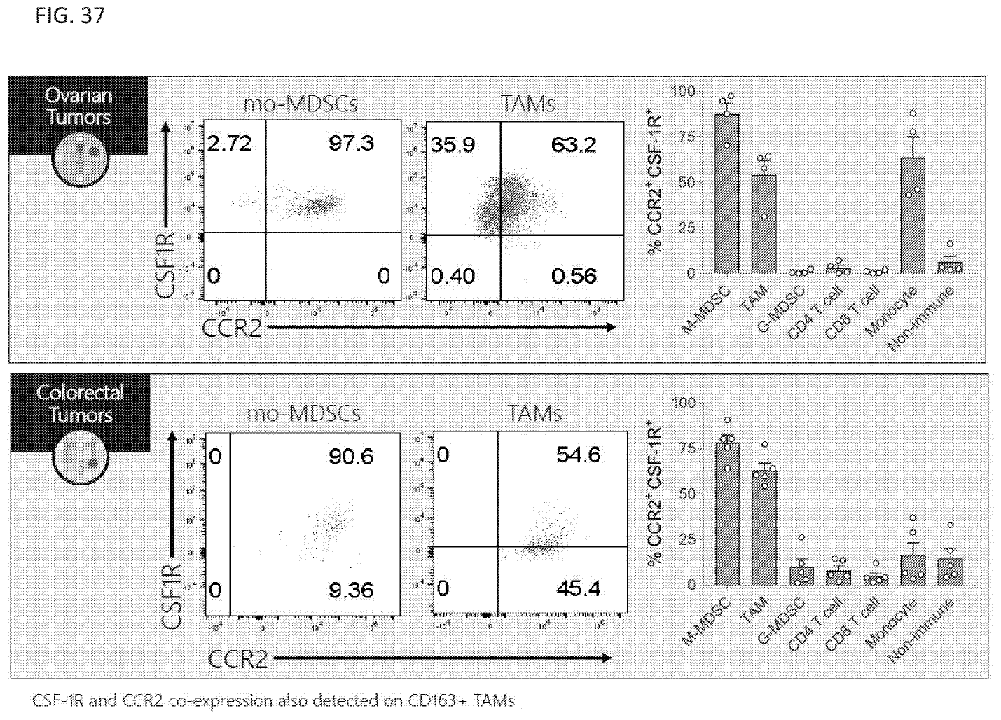

This study examines three TGFβ-trap constructs for their ability to inhibit TGFβ signaling. The first construct, “Single TGFβ Fab-trap” shown in FIG. 36, comprises two chains: the first chain comprises from N-terminus to C-terminus a first TGFBR2 ECD, a first linker, and a heavy chain constant region 1 (CH1); and the second chain comprises from N-terminus to C-terminus a second TGFBR2 ECD, a second linker, and a light chain constant region (CL). This construct does not comprise any targeting domains. The second construct, “Anti-PDL1×TGFβ-trap” shown in FIG. 36, comprises an anti-PDL1 antibody fused, at the C-terminus of its two Fc regions, to a TGFBR2 ECD homodimer. The third construct, “UniTI-102 (Anti-CCR2×anti-CSF1R×TGFβ-trap)” shown in FIG. 36, comprises an anti-CCR2×anti-CSF1R bispecific antibody fused, at the C-terminus of its two Fc regions, to a TGFBR2 ECD homodimer. In addition, a fourth construct, “UniTI-01 (Anti-CCR2×anti-CSF1R)” in FIG. 36, which is an anti-CCR2×anti-CSF1R bispecific antibody without a TGFβ-trap, was used as a negative control.

Briefly, HEK-Blue TGF-b cells were treated with the four constructs described above in a dose dependent manner in the presence of 0.5 ng/ml of TGF-β1 for 20-22 hours. TGF-β1 binds to receptors on HEK-Blue cells and induces activation of the TGF-β/Smad pathway leading to the formation of a Smad3/Smad4 complex. This heterocomplex enters the nucleus and binds SBE (Smad3/4-binding elements) sites inducing production of SEAP (secreted embryonic alkaline phosphatase). SEAP secreted in the supernatant was quantified by colormetric enzymatic assays (QUANTI-Blue). As shown in FIG. 36, TGF-β1-mediated SEAP production was reduced by all three TGFβ-trap constructs tested here. The anti-CCR2×anti-CSF1R bispecific antibody without a TGFβ-trap did not reduce TGF-β1 signaling (FIG. 36).

### Example 28: Characterization of UniTI-102 Constructs

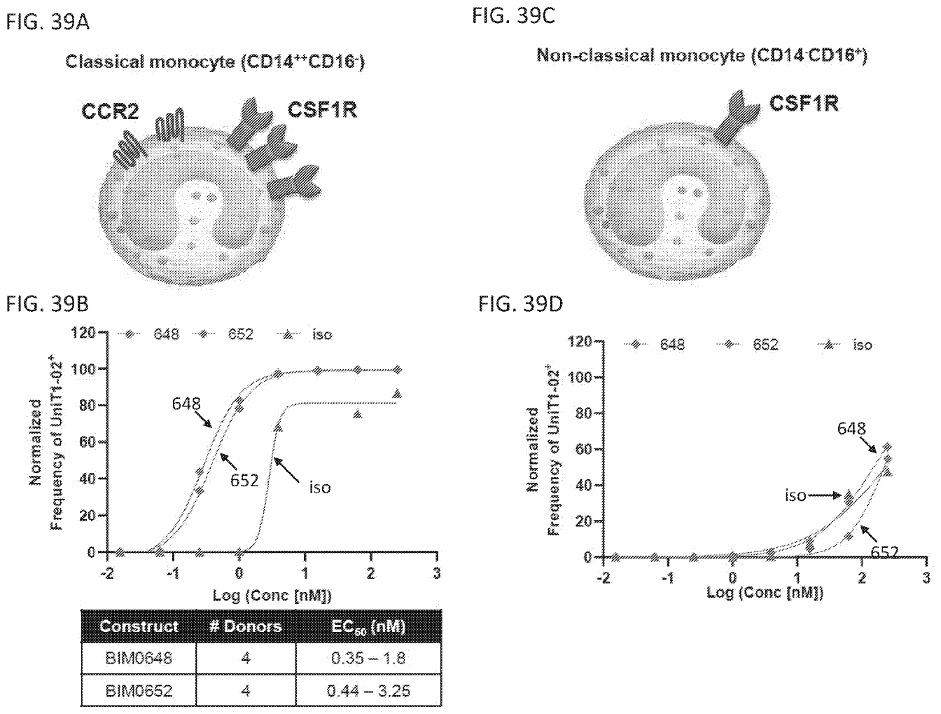

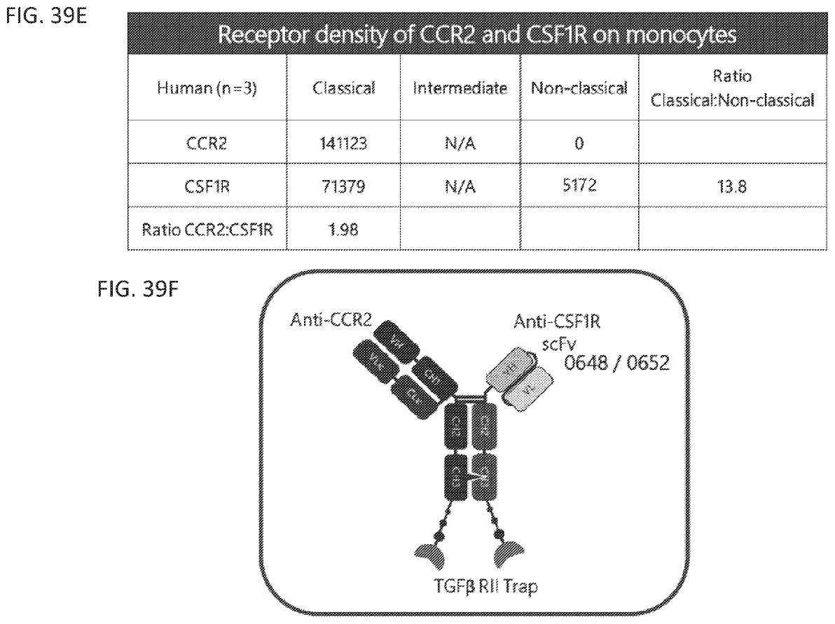

In some embodiments, provided herein are multispecific molecules having the configuration shown in FIG. 12J or 39F. In some embodiments, the multispecific molecules comprise an anti-CSF1R binding moiety (e.g., as an scFv), an anti-CCR2 binding moiety (e.g., as a Fab), and a TGFβ Trap (e.g., a TGFβRII homodimer) fused to the C-terminus of a Fc region. Such molecules are referred to as UniTI-102. Without being bound by theory, UniTI-102 may preferentially bind to CSF1R+ CCR2+Mo-MDSCs and TAMs, while exerting inhibitory functions; prevent recruitment of new MDSCs and TAMs; neutralize locally secreted TGFβ and reprogram the stroma to enable T cell infiltration; and/or restore cytotoxic function of infiltrating T cells and NK cells.

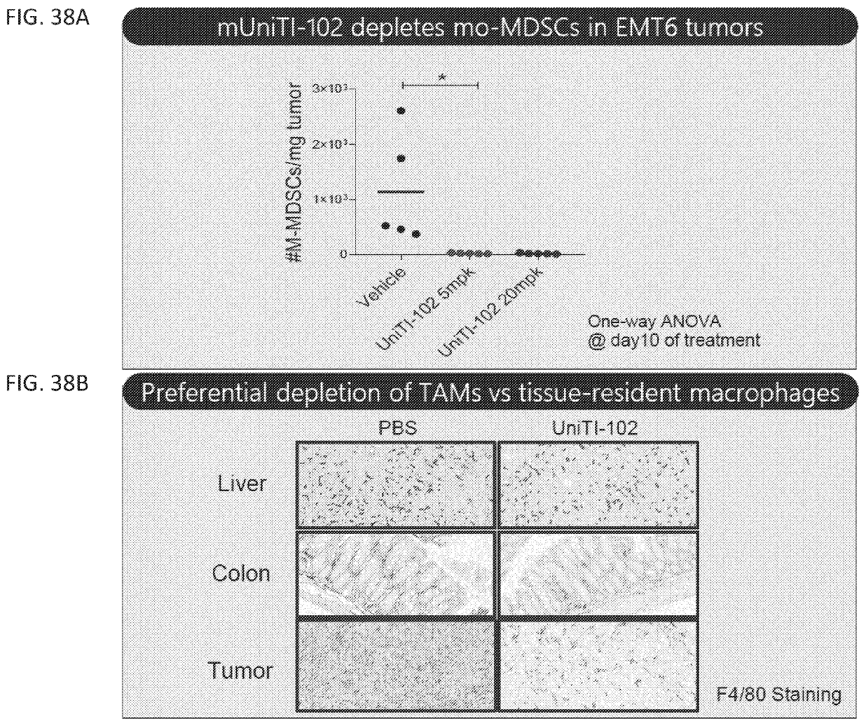

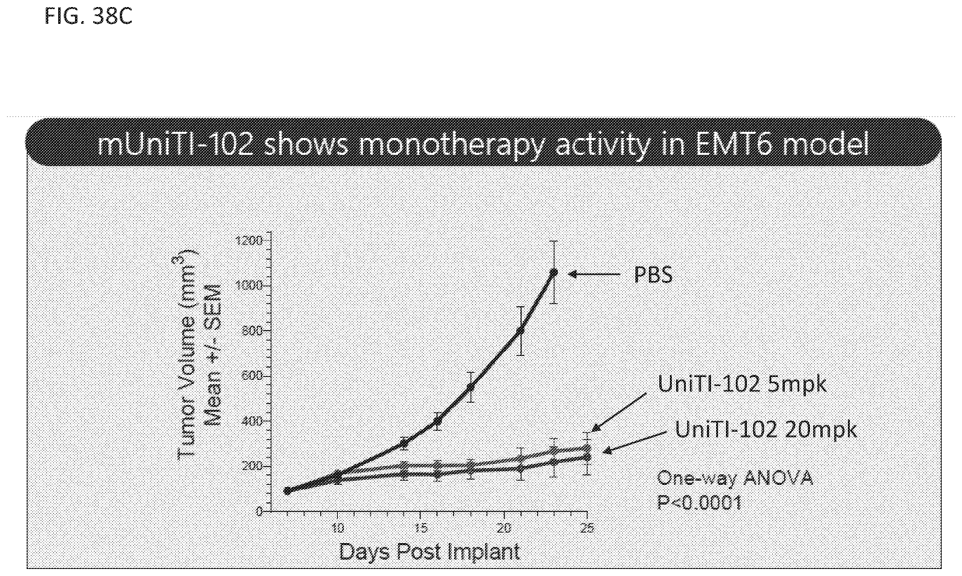

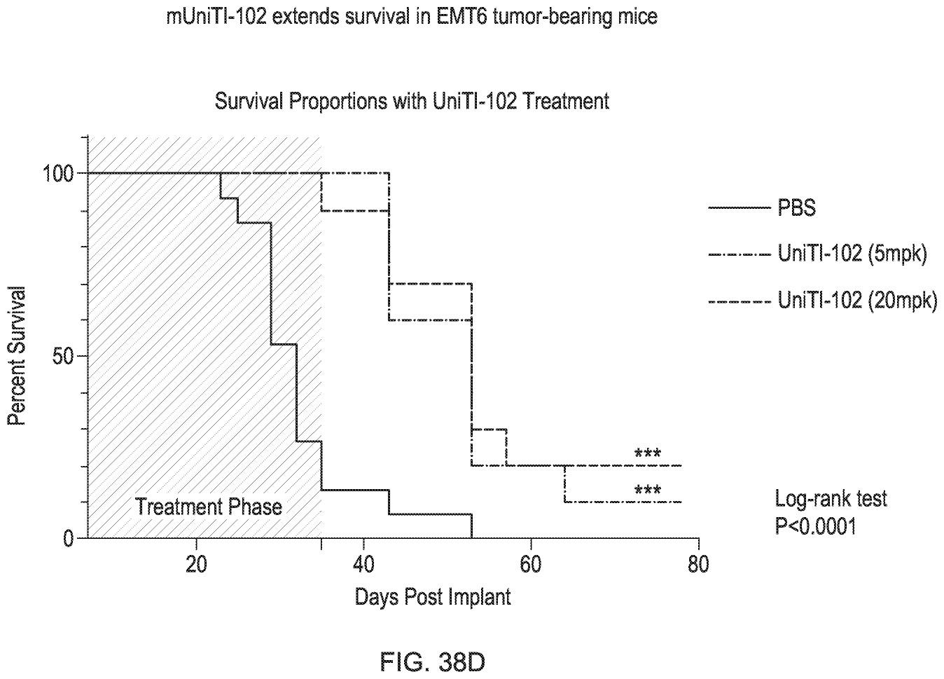

First, a mouse surrogate antibody mUniTI-102 was tested in the EMT6 breast tumor model, which is a syngeneic tumor model resistant to PD1 or TGFβ blockade therapy. mUniTI-102 was administered to EMT6 tumor-bearing B-cell deficient Jh−/− mice at 5 mpk, TIW or 20 mpk BIW. mUniTI-102 depleted mo-MDSCs in EMT6 tumors (FIG. 38A) and preferentially depleted TAMs over tissue-resident macrophages (FIG. 38B). In addition, mUniTI-102 demonstrated anti-tumor activity in this model as a monotherapy (FIGS. 38C and 38D).

The next few studies examine two UniTI-102 molecules, BIM0648 (also referred to as “0648” or “648”) and BIM0652 (also referred to as “0652” and “652”). Both BIM0648 and BIM0652 have the configuration shown in FIG. 39F. The sequence information of these two molecules is disclosed in Table 34. BIM0648 comprises the amino acid sequences of SEQ ID NO: 127, SEQ ID NO: 131, and SEQ ID NO: 373. BIM0652 comprises the amino acid sequences of SEQ ID NO: 136, SEQ ID NO: 131, and SEQ ID NO: 373.

The binding of BIM0648 and BIM0652 to whole blood was examined using flow cytometry. Briefly, peripheral blood was collected in tubes containing lithium heparin as anticoagulant from 4 healthy controls. 100 μl of blood was added to each tube and appropriate antibodies (Table 35) were added before incubation in the dark for 30 minutes at 4° C. UniTI-102 constructs were fluorescently labeled with AlexaFlour647 as per manufacturer's instructions (Molecular Probes Inc, Eugene Oreg.) and added to tubes with the antibodies. Effective labeled construct concentrations ranged from 1000 nM to 62.5 μM. Red blood cells were lysed by the addition of Ammonium Chloride (StemCell Technologies, Cambridge Mass.). After lysis, the cells were washed in PBS containing 1% Fetal Bovine Serum and fixed in 1% paraformaldehyde. Samples were run on the Beckman Coulter Cytoflex LX. Instrument settings (cytosettings; unique voltage and compensation matrices) were identical for all donors examined. Raw data was analyzed using FlowJo 10. Single stains and fluorescence minus one (FMO) control tubes were used to define positive/negative populations. All data analysis and graphical representation of the data was performed using Prism 8.1 (GraphPad Software).

TABLE 35
Antibody panel used for cell staining.

| | Marker | Channel | Clone | Vendor |
|---|---|---|---|---|
| 1 | CD3 | BV605 | SP34-2 | BioLegend |
| 2 | CD20 | BV785 | HIB19 | BioLegend |
| 3 | CD56 | PE | 5.1.H11 | BioLegend |
| 4 | CD14 | BV421 | M5E2 | BioLegend |
| 5 | CD16 | FITC | 3G8 | Ancell Corporation |
| 6 | Live/Dead | NUV450 | — | BioLegend |
| 7 | UniT102 | Alexa Fluor 647 | — | — |

The examination of binding ex vivo in blood (prior to lysis) precluded the need for Fc blocking agents. Additionally, the use of fluorescently labeled probes eliminated the need for secondary detection reagents. Leukocytes were first isolated from lysed RBCs by forward and side scatter. Live cells were gated followed by exclusion of doublets via FSC-A against FSC-H plot which was followed by gating on live single cell populations. Granulocytes were separated from lymphocytes (Low SSC) and monocytes (medium SSC). CD3 and CD56 bivariate plot allowed discrimination of T (CD3+), NK (CD56+) and NK-T (CD3+CD56+) cell populations. From the CD3/CD56 negative cells, CD14 and CD16 bivariate plot was generated for monocytes (CD14+CD16+ classical monocytes, CD14−CD16+ non classical monocytes and CD14+CD16+ intermediate monocytes). Overall, the combination of the 6-marker panel with the fluorescently labeled UniTI-102 constructs allowed examination of binding of UniTI-102 constructs as percentages of parents and grandparent populations in a total of 5 different primary cell populations: granulocyte, T, NK, B and monocytes. The protocol used enabled quantitation of monocytes and granulocytes in addition to lymphocyte populations.

Both BIM0648 and BIM0652 preferentially bound to primary human classical monocytes in whole blood (FIGS. 39B and 39D). Binding to intermediate monocytes was observed and was weaker than binding to classical monocytes. UniTI-102 molecules showed minimal to no engagement with T, B, NK or granulocytes in whole blood (data not shown).

The ability of BIM0648 and BIM0652 to neutralize TGFβ was confirmed by various in vitro functional assays described below.

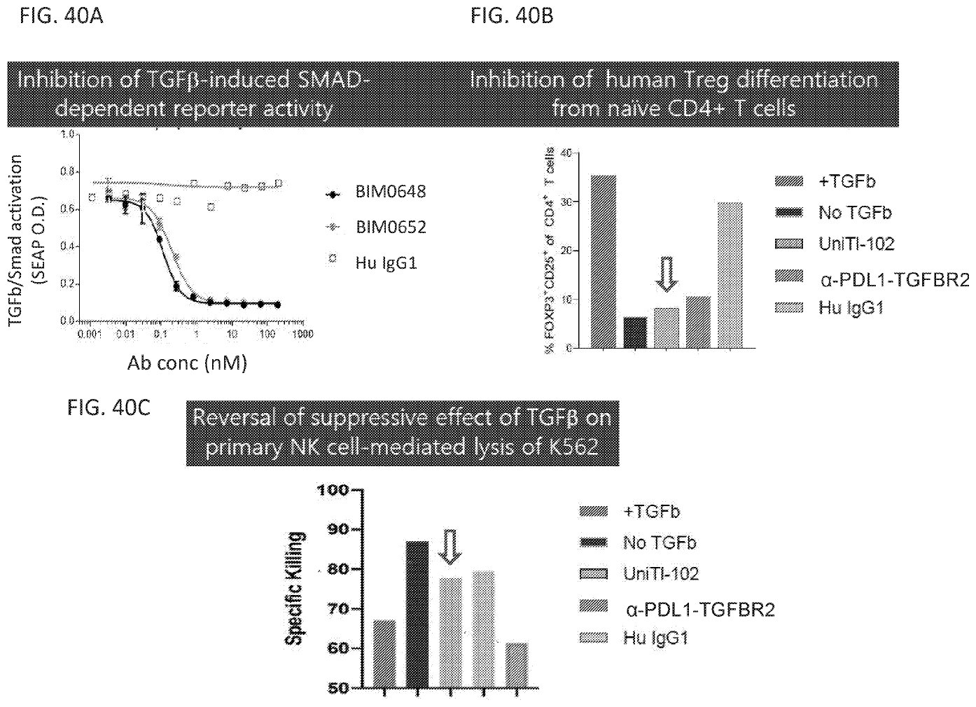

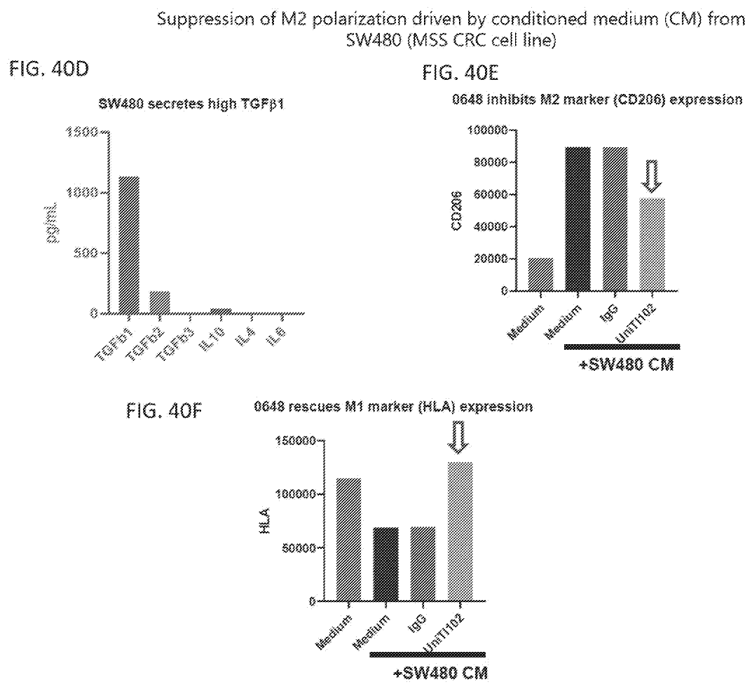

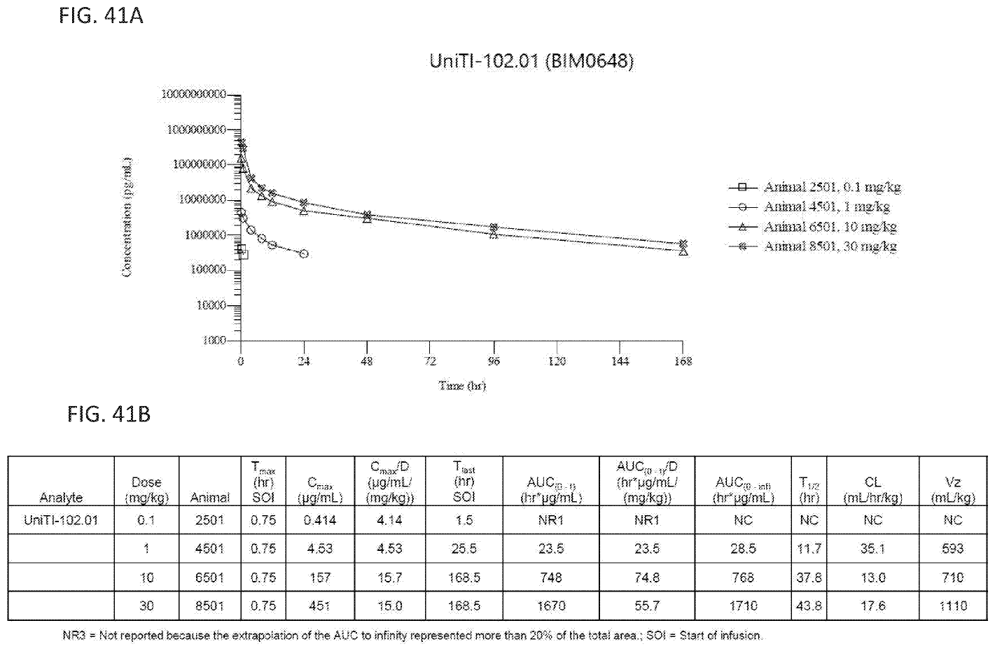

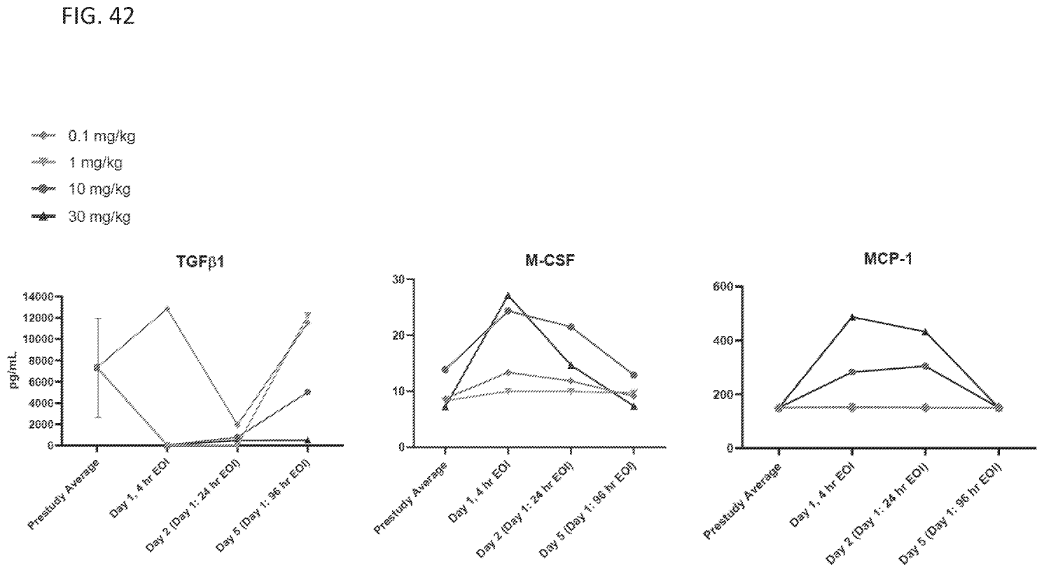

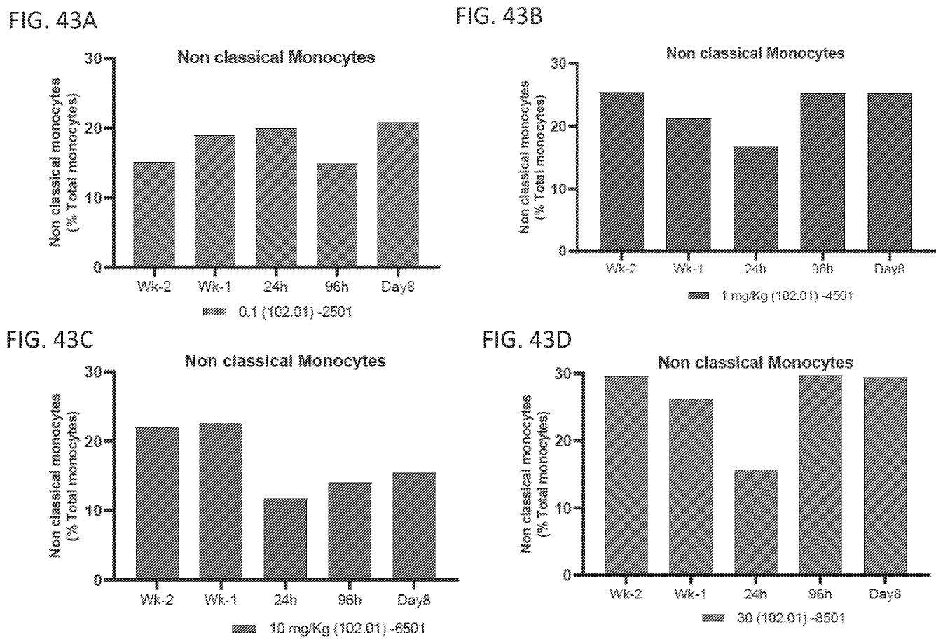

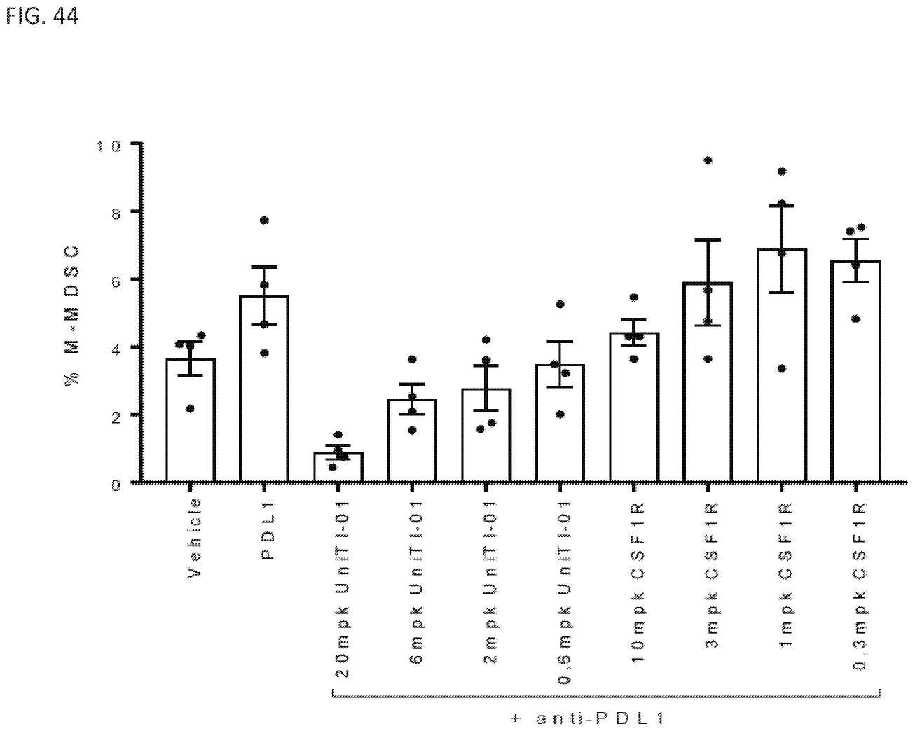

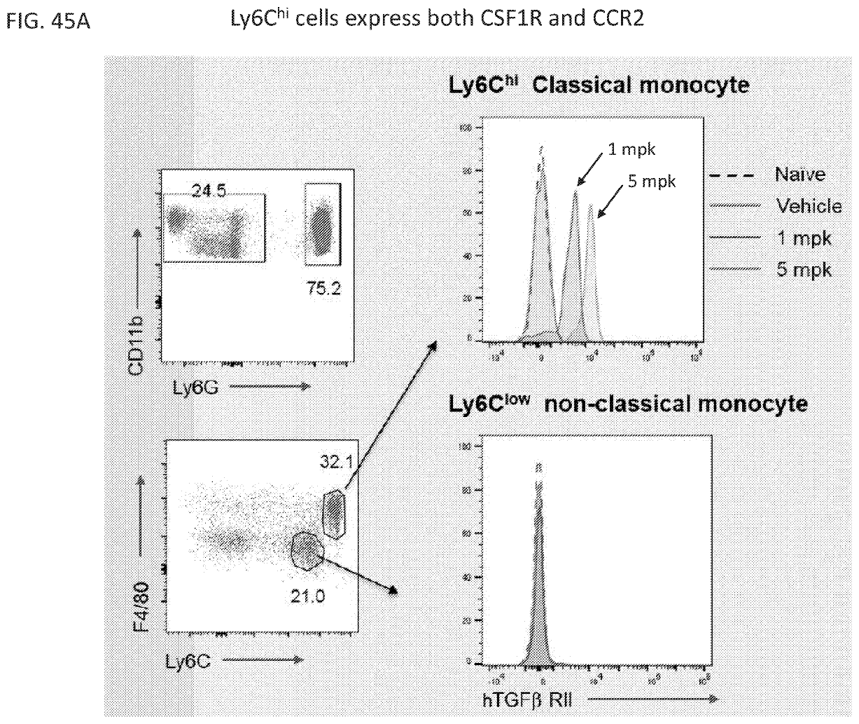

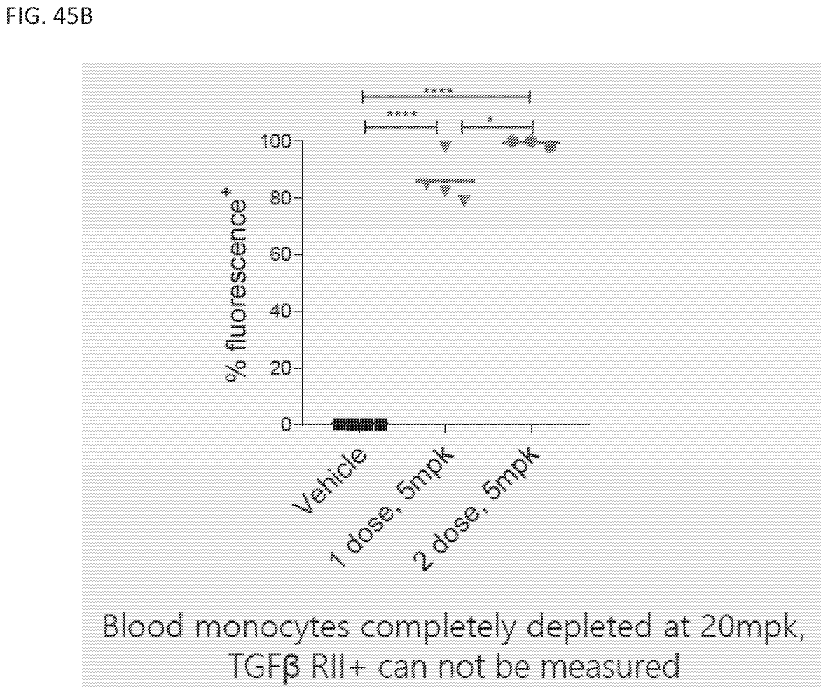

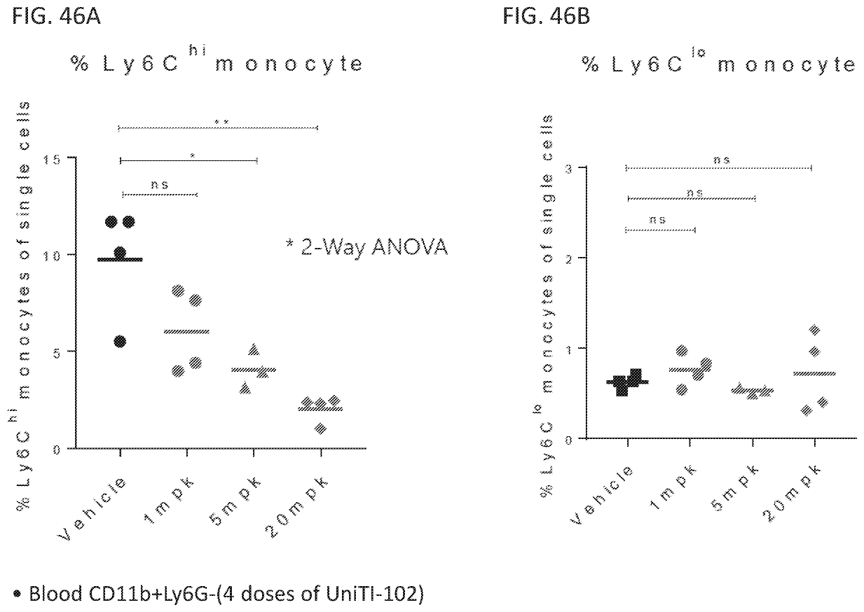

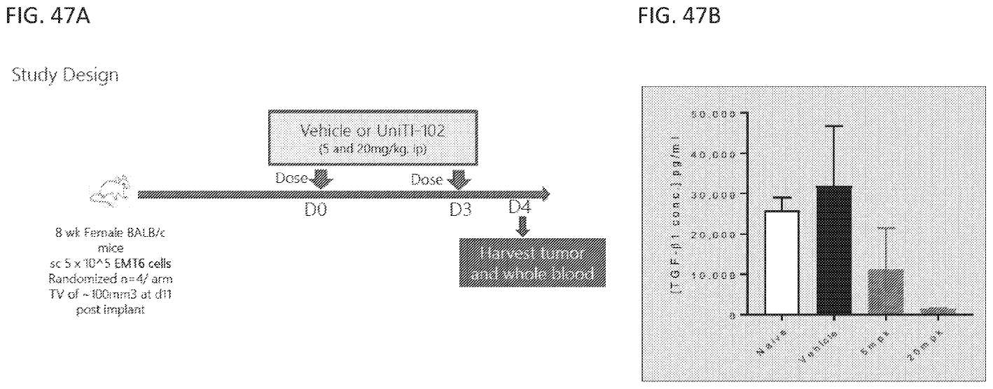

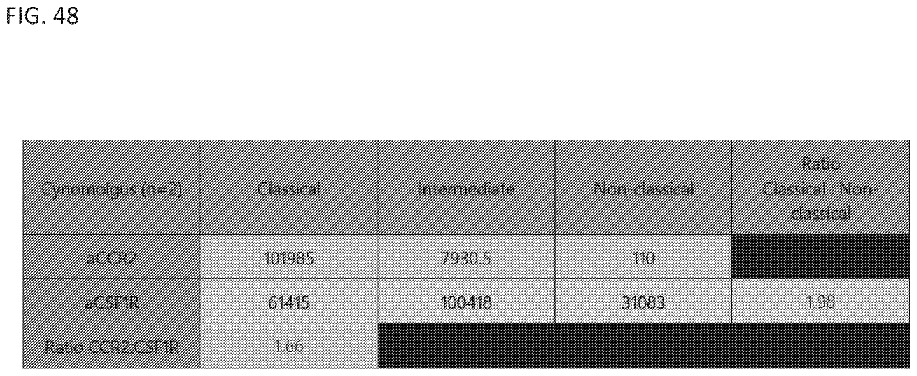

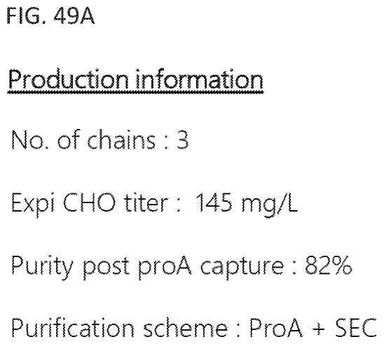

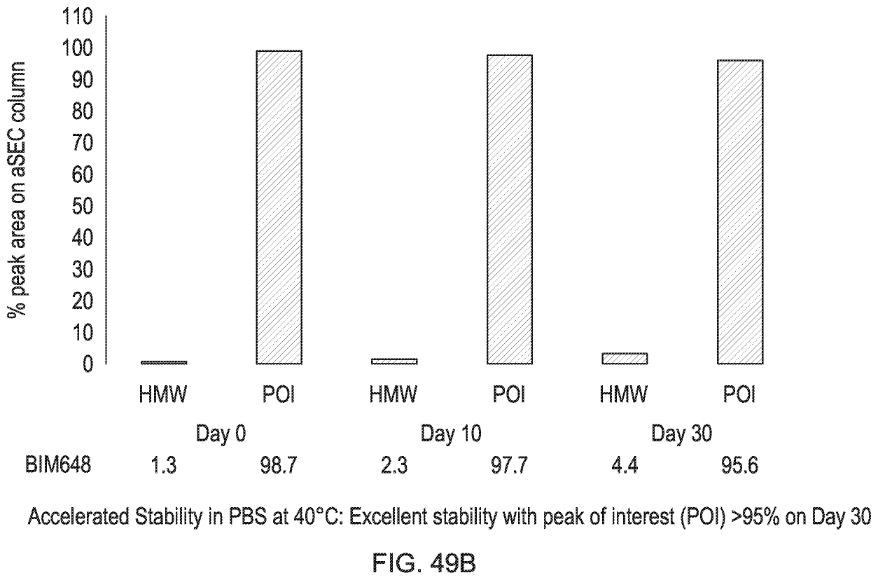

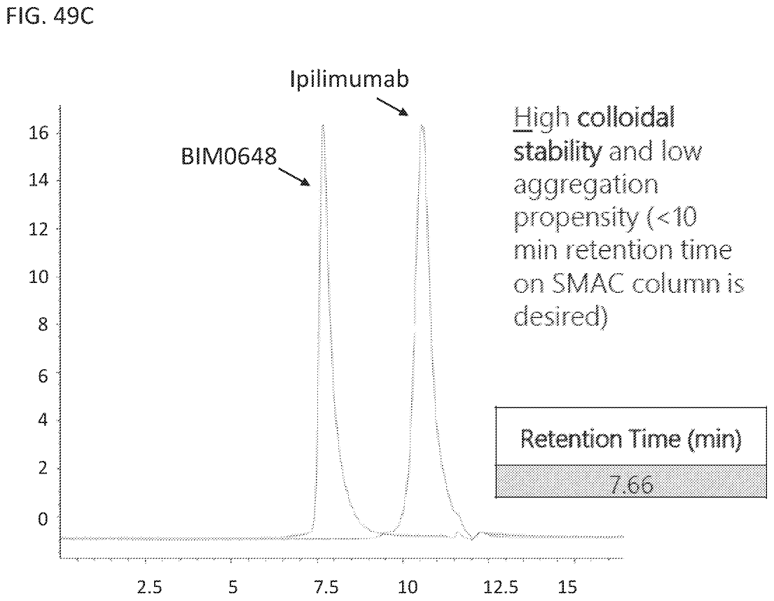

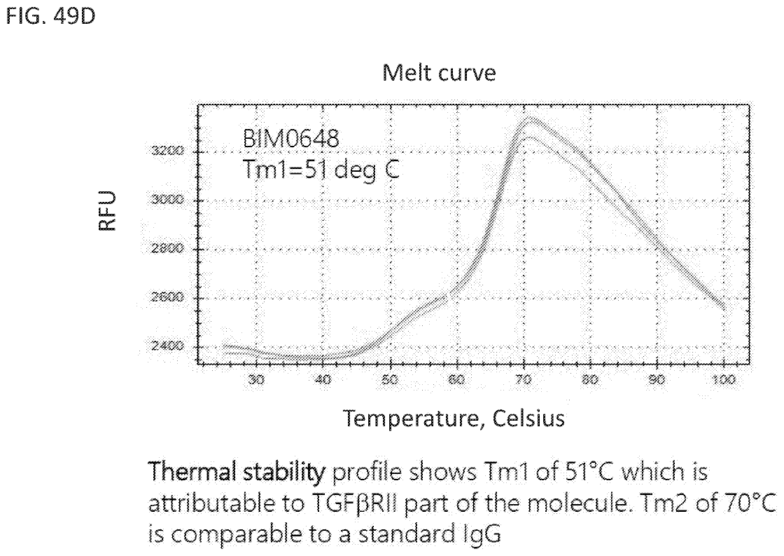

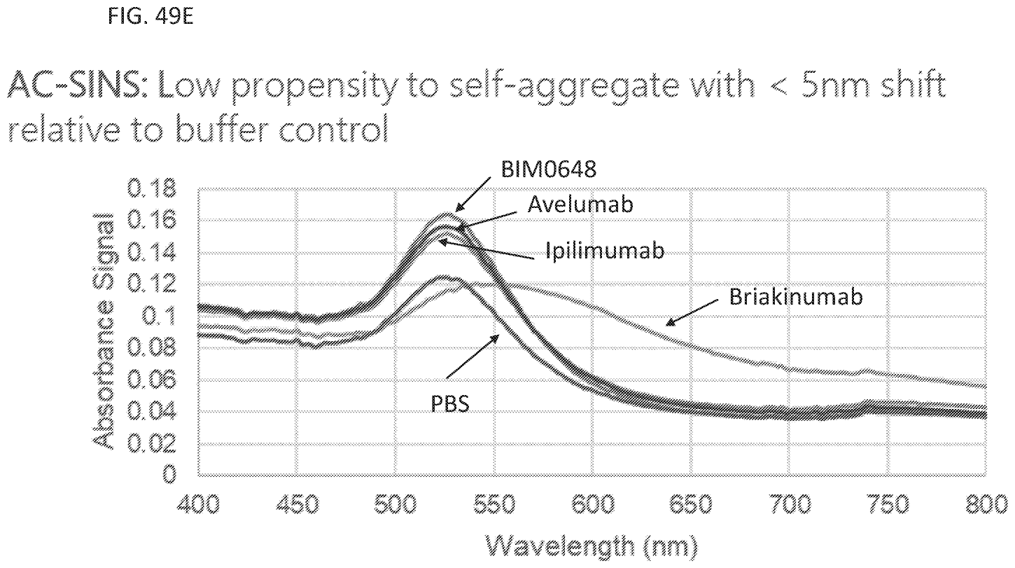

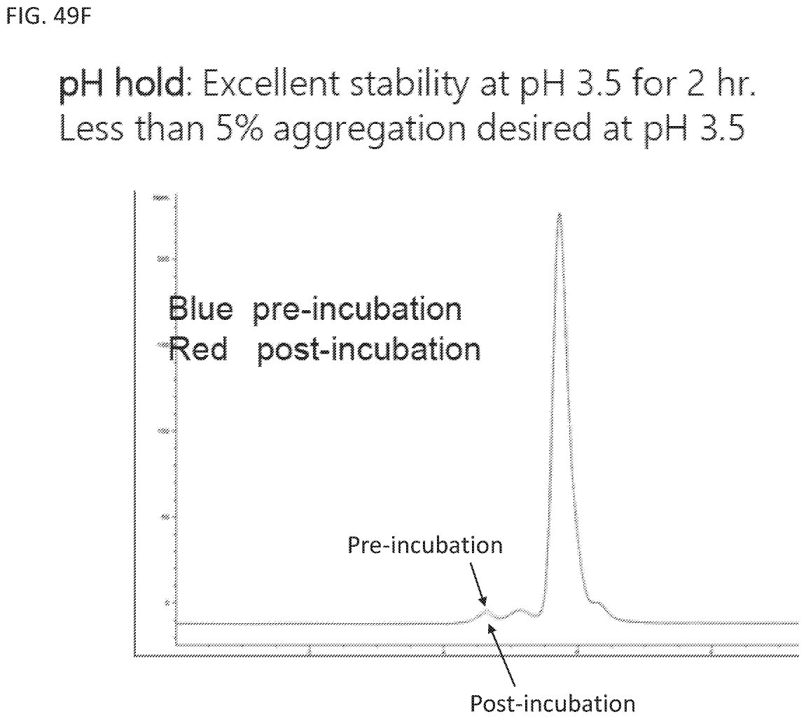

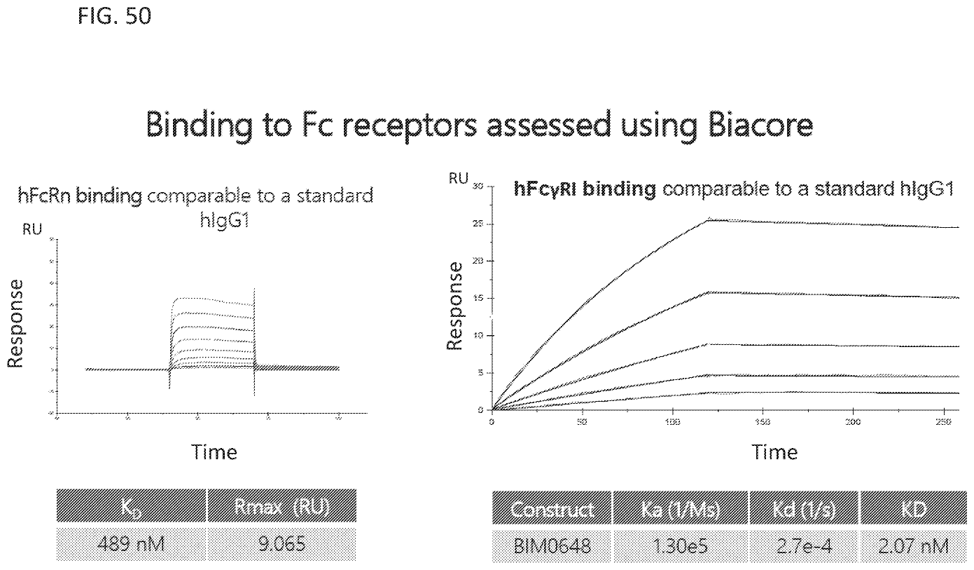

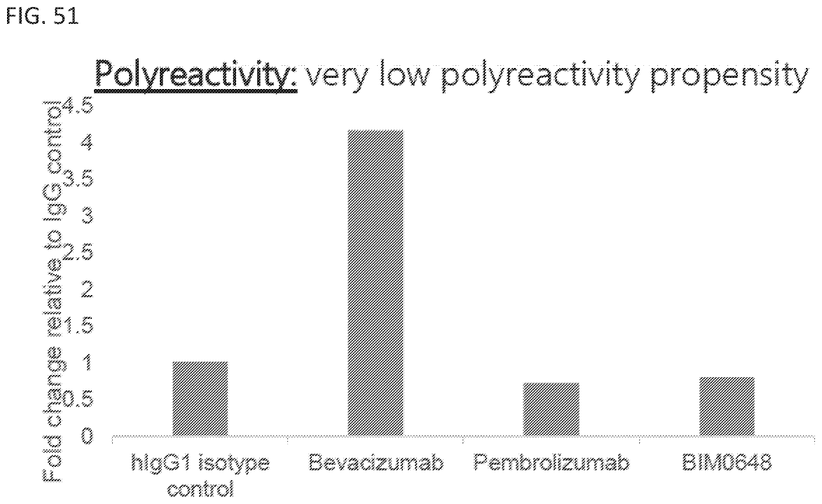

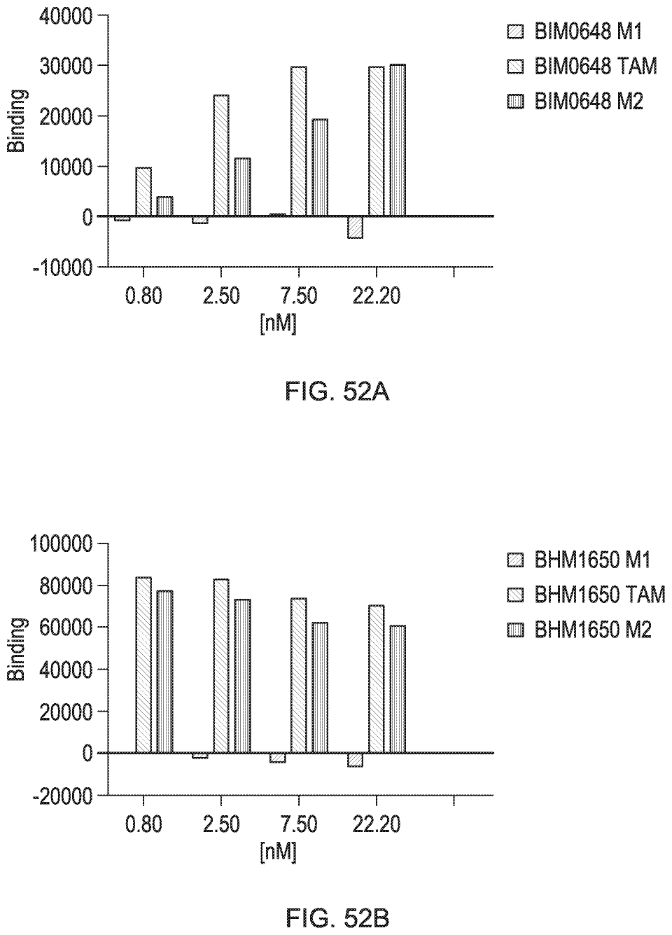

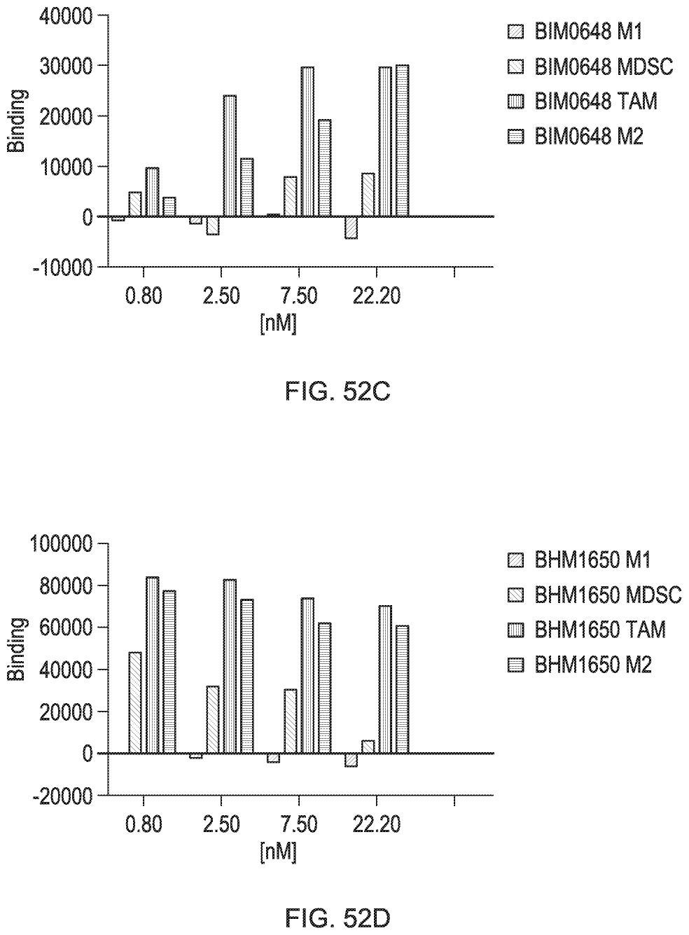

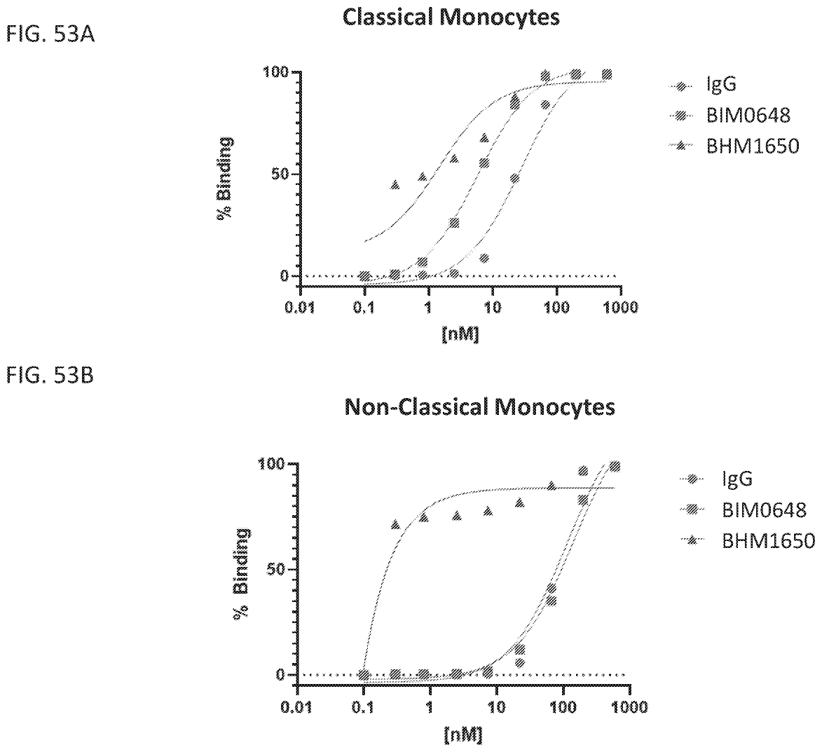

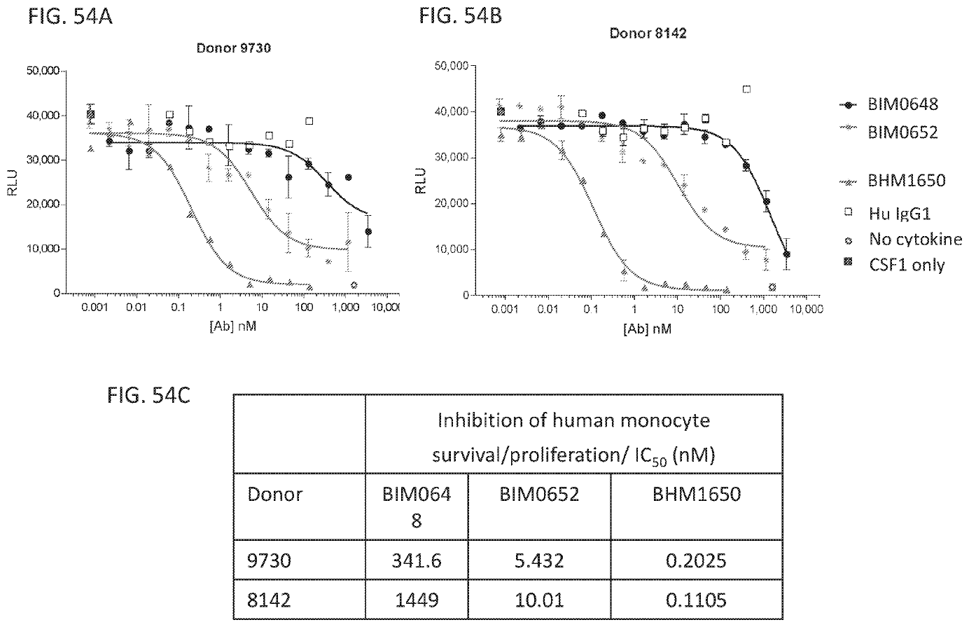

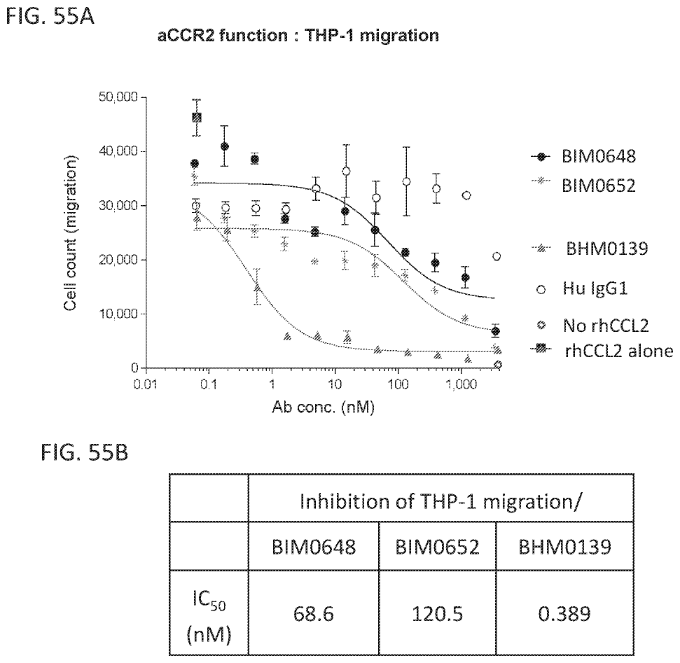

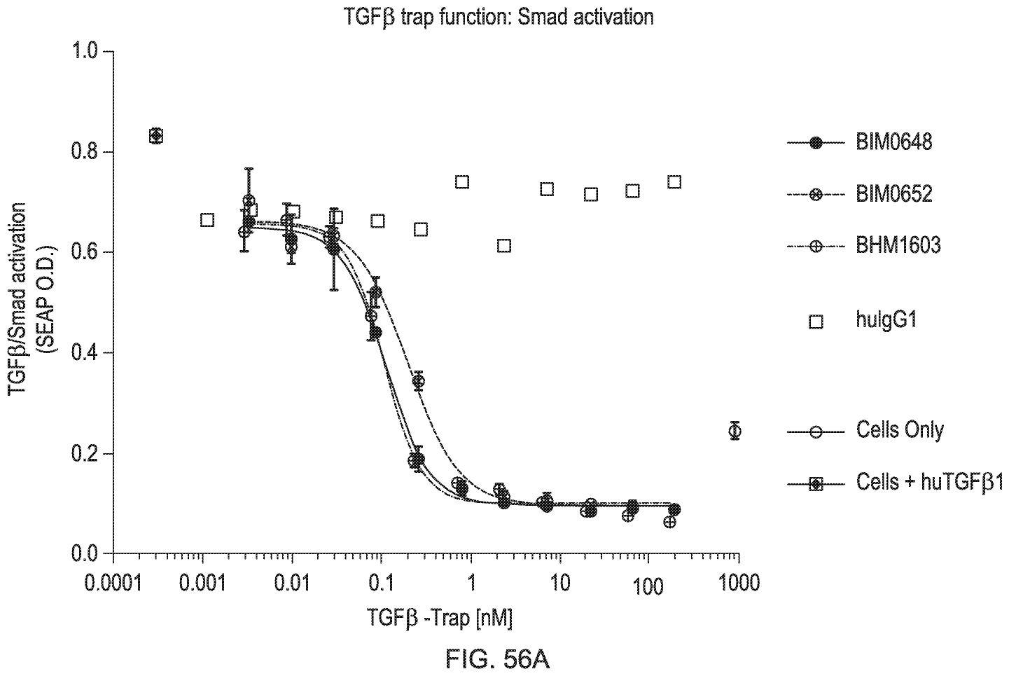

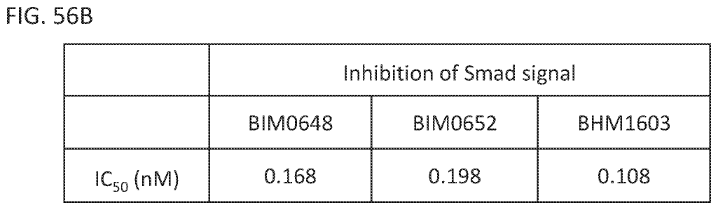

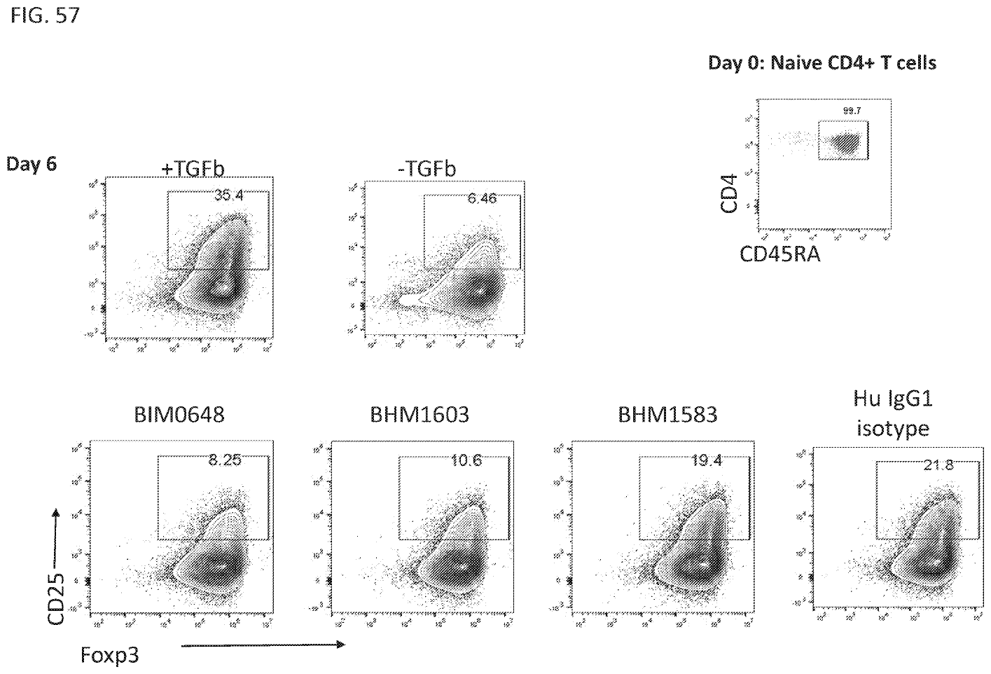

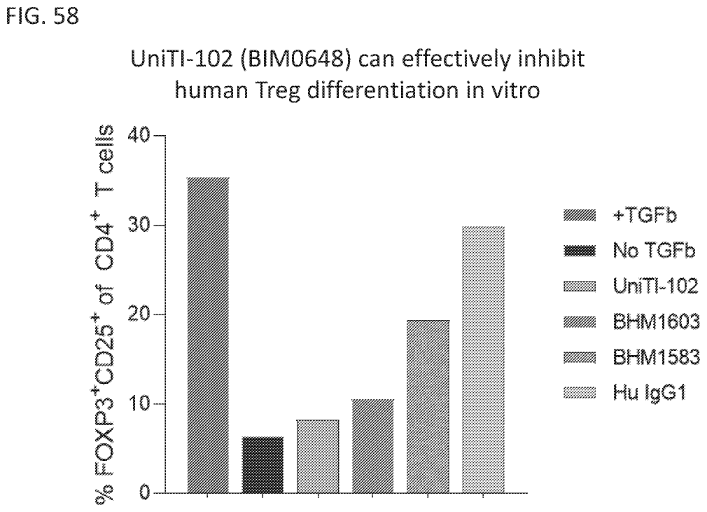

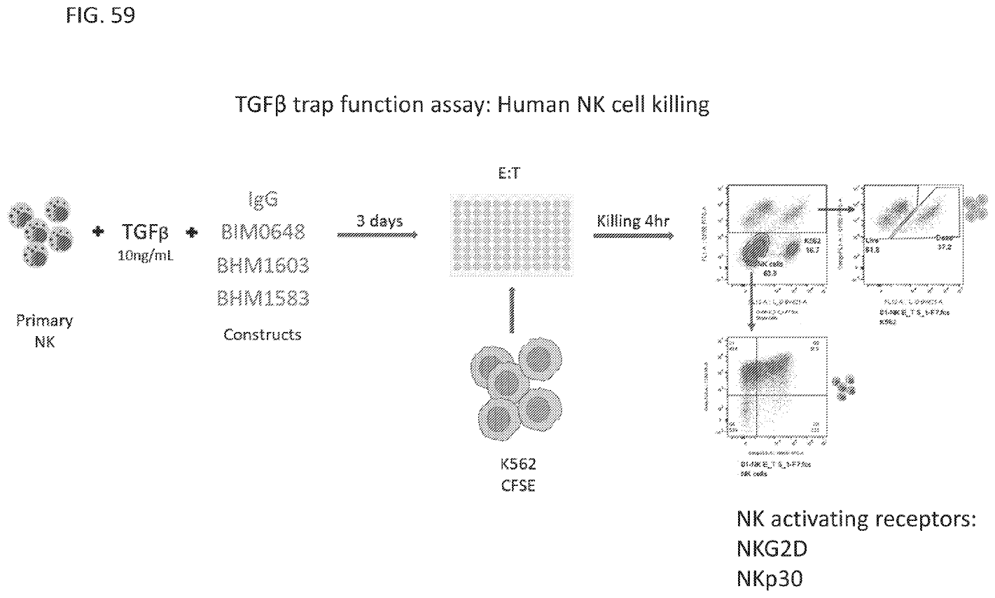

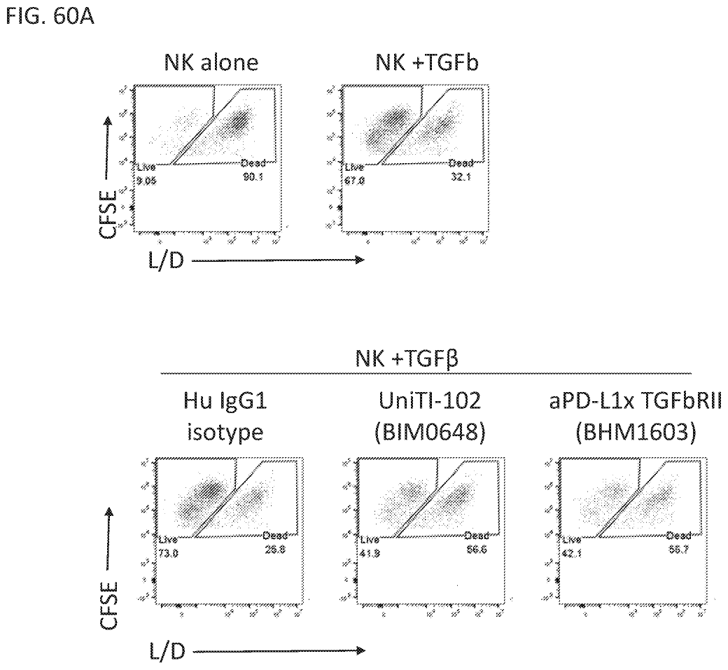

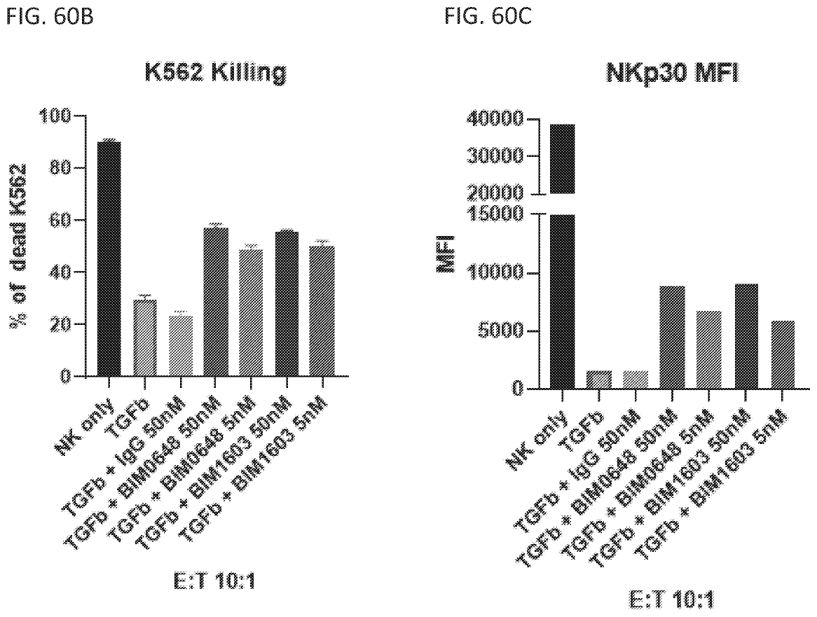

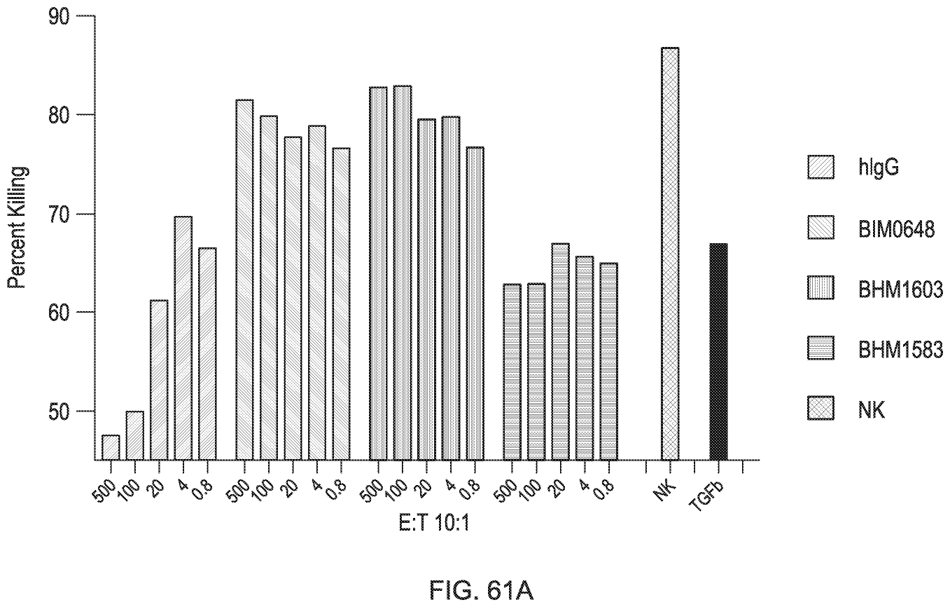

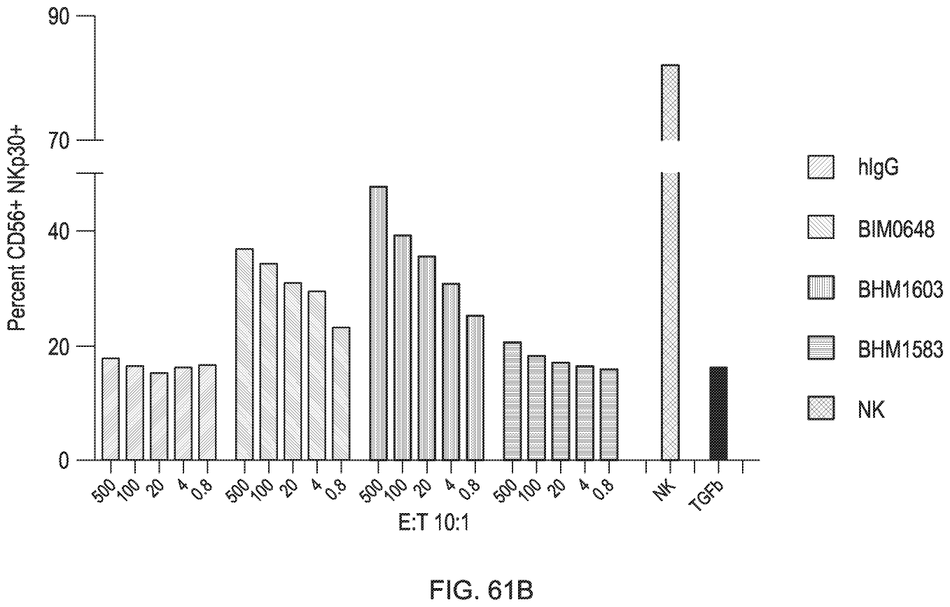

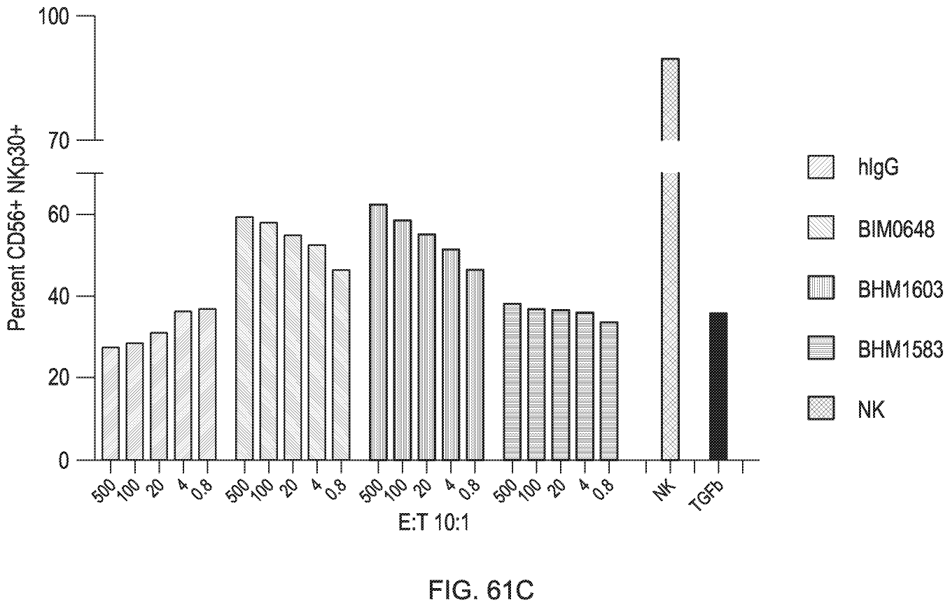

Both BIM0648 and BIM0652 inhibited TGFβ-induced SMAD-dependent reporter activity (FIG. 40A).

To test the ability of UniTI-102 to inhibit human Treg differentiation from naïve CD4+ T cells, human naïve CD4+ T cells were isolated from fresh PBMCs, and 150,000 cells/well were added to a 96-well round bottom plate pre-coated with anti-CD3 Ab (OKT3; 5 μg/mL). Cells were cultured in X-VIVO15 media with 2 mM L-glutamine, IL-2 (50 ng/mL), TGFβ (10 ng/mL), and anti-CD28 Ab (1 μg/mL) for 6-days at 37° C., with the following constructs: UniTI-102, anti-PD-L1/anti-TGFβ-Trap, or human IgG1 at 6.2 nM. Controls included cells without constructs: with TGFβ (TGFβ+) for the maximum induction of T-reg differentiation, and without TGFβ (TGFβ−) for baseline inhibition. On day 6, cells were washed and stained with viability dye followed by an extracellular antibody cocktail (anti-CD3, anti-CD4, and anti-CD25). Cells were then fixed, permeabilized, and stained for intracellular foxp3 with an anti-Foxp3 Ab (150D; 1:50 dilution). Samples were acquired on Beckman Coulter Cytoflex machine and gated on live, CD3+, CD4+, and CD25+/Foxp3+ cells. As shown in FIG. 40B, UniTI-102 inhibited TGFβ-induced human Treg differentiation from naïve CD4+ T cells.

Next, UniTI-102 was tested for its ability to reverse the suppressive effect of TGFβ on primary NK cell-mediated lysis of K562 cells. Briefly, frozen primary NK cells were thawed and rested overnight in X-VIVO15 medium supplemented with 20% FBS and 100 IU/mL recombinant human IL-2. Cells were then seeded at 1×10⁶ cells/ml and treated for 3 days with TGFβ (10 ng/mL) alone, or TGFβ with (i) UniTI-102, (ii) anti-PD-L1-TGFβ-Trap, or (iii) human IgG1 at 20 nM. On day 3, treated NK cells were counted, and incubated with CFSE-labeled K562 cells at E:T ratio of 10:1 for 4 hours. Cells were then stained with viability dye followed by an antibody cocktail (anti-CD56, anti-NKp30 and anti-NKG2). Samples were acquired on Beckman Coulter Cytoflex machine and analyzed by FlowJo software. As shown in FIG. 40C, TGFβ reduced primary NK cell-mediated lysis of K562 cells and this suppressive effect of TGFβ could be reversed by UniTI-102.

Furthermore, a study was conducted to examine the ability of UniTI-102 to suppress M2 polarization driven by conditioned medium (CM) from SW480 (MSS CRC cell line). To generate tumor-conditioned medium (TCM), SW840 colon cancer cells (Cat #CCL-228, ATCC) were seeded in 10% FBS/RPMI1640 medium and after 24 hours were switched to 0.2% FBS/RPMI1640 medium. Medium was collected after 48 hours, filtered, restored to 10% FBS, and was used fresh, or frozen at −80° C. Monocytes were isolated from healthy donor PBMCs (AllCells) by classical monocyte isolation kit (Cat #130-117-337, Miltenyi Biotec), and seeded in SW480 TCM with 50 ng/ml M-CSF (Cat #300-25, PeproTech) at 1×10⁶/ml in 2 ml/well in 6-well plates. After 3 days, the medium was replenished with 1 ml SW480 TCM/50 ng/ml M-CSF. Monocytes were treated with UniTI-102 or human IgG1 (Cat #403502, BioLegend) for 48 hours. Cells were harvested by treatment with Versene (Cat #15040066, ThermoFisher), incubated with Zombie UV viability dye (Cat #423108, BioLegend), incubated with fluorophore-conjugated CD206 and HLA antibodies (both from Biolegend), acquired on CytoFLEX (Beckman Coulter) flow cytometer, and analyzed by FlowJo software. SW840 colon cancer cells secreted high levels of TGFβ1 (FIG. 40D), which drives M2 polarization. BIM0648 suppressed SW840 CM-induced M2 polarization as evidenced by a reduction in CD206 (a M2 marker) expression (FIG. 40E) and an increase in HLA (a M1 marker) expression (FIG. 40F).

In some embodiments, UniTI-102 can be used for the treatment of microsatellite-stable (MSS) colorectal cancer. In CRC patients, increased TGFβ1 levels may predict adverse outcomes (Calon et al., Nat Genet. 2015 April; 47(4):320-9). CRC recurrence and metastasis strictly depends on high TGFβ signaling (Calon et al., Cancer Cell. 2012 Nov. 13; 22(5):571-84); and deletion of Tgfbr2 in myeloid cells significantly inhibits tumor metastasis in preclinical models (Pang et al., Cancer Discov. 2013 August; 3(8):936-51). An immune subset of MSS GI tumors, including CRC, consists of CD163+ myeloid cells with high CCR2, CSF1R and TGFβ1 expression. Majority of PD-L1 is expressed by myeloid cells in CRC MSI patients (Llosa et al., Cancer Discov. 2015 January; 5(1):43-51). MSS CRC patients express low to undetectable PD-L1. Metastasis associated TAMs maintain CCR2 and CSF1R co expression.

Without being bound by theory, UniTI-102 may induce/restore anti-tumor response in the MSS CRC via complementary and/or synergistic mechanisms. For example, neutralization of locally secreted TGFβ by UniTI-102 may reprogram the stroma to enable immune cell infiltration. Co-inhibition of CCR2 and CSF1R by UniTI-102 may prevent infiltration of new monocytes derived immunosuppressive myeloid cells due to TGFβ inhibition.

In some embodiments, UniTI-102 may be combined with chemo/radiation, anti-angiogenic or immune checkpoint therapies. Radiation therapy, chemotherapy or anti-VEGF therapies are shown to recruit TAMs and MDSCs and increase TGFβ levels, which are associated with tumor recurrence and/or resistance. Blood based biomarkers for demonstrating target engagement and relevant biological responses are useful in early clinical trials.

In summary, UniTI-102 molecules are tri-specific molecules simultaneously targeting CCR2, CSF1R and TGFβ. UniTI-102 molecules preferentially bind to CCR2 and CSF1R double positive cells: mo-MDSCs, TAMs, and classical monocytes. All three arms of UnITI-102 are capable of functionally blocking the intended targets. Cynomolgus monkey studies confirmed target engagement for all three arms of UniTI-102, with biological responses consistent with CSF1R blockade in vivo.

### INCORPORATION BY REFERENCE

All publications and patents mentioned herein are hereby incorporated by reference in their entirety as if each individual publication or patent was specifically and individually indicated to be incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1.-81. (canceled)

82. A multispecific molecule comprising:
(i) an anti-CSF1R binding moiety;
(ii) an anti-CCR2 binding moiety; and
(iii) a TGF-beta inhibitor,
wherein the anti-CSF1R binding moiety is an anti-CSF1R antibody molecule or antigen-binding fragment thereof, and the anti-CCR2 binding moiety is an anti-CCR2 antibody molecule or antigen-binding fragment thereof.

83. The multispecific molecule of claim 82, wherein the anti-CSF1R binding moiety comprises:
(a) a heavy chain variable region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3,
wherein the HCDR1, the HCDR2, the HCDR3, the LCDR1, the LCDR2, and the LCDR3 comprise the amino acid sequences of:
SEQ ID NOs: 402, 474, 475, 432, 434, and 436, respectively;
SEQ ID NOs: 402, 405, 413, 432, 434, and 436, respectively;
SEQ ID NOs: 402, 404, 413, 432, 434, and 436, respectively;
SEQ ID NOs: 402, 406, 413, 432, 434, and 436, respectively;
SEQ ID NOs: 402, 404, 414, 432, 434, and 436, respectively;
SEQ ID NOs: 402, 407, 413, 432, 434, and 436, respectively;
SEQ ID NOs: 402, 404, 415, 432, 434, and 436, respectively;
SEQ ID NOs: 402, 408, 413, 432, 434, and 436, respectively;
SEQ ID NOs: 402, 404, 416, 432, 434, and 436, respectively;
SEQ ID NOs: 402, 404, 417, 432, 434, and 436, respectively;
SEQ ID NOs: 402, 409, 413, 432, 434, and 436, respectively;
SEQ ID NOs: 402, 404, 418, 432, 434, and 436, respectively;
SEQ ID NOs: 402, 404, 419, 432, 434, and 436, respectively;
SEQ ID NOs: 402, 404, 420, 432, 434, and 436, respectively;
SEQ ID NOs: 402, 404, 421, 432, 434, and 436, respectively;
SEQ ID NOs: 403, 478, 479, 433, 435, and 437, respectively;
SEQ ID NOs: 403, 411, 422, 433, 435, and 437, respectively;
SEQ ID NOs: 403, 410, 422, 433, 435, and 437, respectively;
SEQ ID NOs: 403, 410, 423, 433, 435, and 437, respectively; or
SEQ ID NOs: 403, 412, 422, 433, 435, and 437, respectively; or
(b) a VH comprising the HCDR1, the HCDR2, and the HCDR3 of SEQ ID NOs: 402, 476, and 477, respectively.

84. The multispecific molecule of claim 82, wherein the anti-CSF1R binding moiety comprises:
(a) a VH comprising an amino acid sequence having at least 80% sequence identity to any one sequence selected from the group consisting of SEQ ID NOs: 323-336 and 339, or an amino acid sequence having at least 90% sequence identity to any one sequence selected from the group consisting of SEQ ID NOs: 337, 338, and 340;
(b) a VH comprising an amino acid sequence encoded by a nucleotide sequence having at least 80% sequence identity to the sequence of SEQ ID NO: 129 or SEQ ID NO: 138;
(c) a VL comprising an amino acid sequence having at least 80% sequence identity to the amino acid sequence of SEQ ID NO: 341 or SEQ ID NO: 139, or an amino acid sequence having at least 90% sequence identity to the sequence of SEQ ID NO: 342;
(d) a VL comprising an amino acid sequence encoded by a nucleotide sequence having at least 80% sequence identity to the sequence of SEQ ID NO: 130 or SEQ ID NO: 140; or
(e) a VH and a VL comprising the amino acid sequences of:
SEQ ID NOs: 324 and 341, respectively;
SEQ ID NOs: 323 and 341, respectively;
SEQ ID NOs: 325 and 341, respectively;
SEQ ID NOs: 326 and 341, respectively;
SEQ ID NOs: 327 and 341, respectively;
SEQ ID NOs: 328 and 341, respectively;
SEQ ID NOs: 329 and 341, respectively;
SEQ ID NOs: 330 and 341, respectively;
SEQ ID NOs: 331 and 341, respectively;
SEQ ID NOs: 332 and 341, respectively;
SEQ ID NOs: 333 and 341, respectively;
SEQ ID NOs: 334 and 341, respectively;
SEQ ID NOs: 335 and 341, respectively;
SEQ ID NOs: 336 and 341, respectively;
SEQ ID NOs: 337 and 342, respectively;
SEQ ID NOs: 338 and 342, respectively;
SEQ ID NOs: 339 and 139, respectively;
SEQ ID NOs: 339 and 342, respectively; or
SEQ ID NOs: 340 and 342, respectively.

85. The multispecific molecule of claim 82, wherein the anti-CSF1R binding moiety comprises:
(a) (i) a VH comprising a HCDR1, a HCDR2, and a HCDR3 of SEQ ID NOs: 402, 405, and 413, respectively, and a VL comprising a LCDR1, a LCDR2, and a LCDR3 of SEQ ID NOs: 432, 434, and 436, respectively; or
(ii) a VH comprising a HCDR1, a HCDR2, and a HCDR3 of SEQ ID NOs: 402, 407, 413, respectively, and a VL comprising a LCDR1, a LCDR2, and a LCDR3 of SEQ ID NOs: 433, 435, 437, respectively; or
(b) (i) a VH comprising an amino acid sequence of SEQ ID NO: 323, and a VL comprising an amino acid sequence of SEQ ID NO: 341; or
(ii) a VH comprising an amino acid sequence of SEQ ID NO: 327, and a VL comprising an amino acid sequence of SEQ ID NO: 342.

86. The multispecific molecule of claim 82, wherein the anti-CCR2 binding moiety comprises:
(a) a VH comprising a HCDR1, a HCDR2, and a HCDR3, and a VL comprising a LCDR1, a LCDR2, and a LCDR3,
wherein the HCDR1, the HCDR2, the HCDR3, the LCDR1, the LCDR2, and the LCDR3 comprise the amino acid sequences of SEQ ID NOs: 446, 447, 448, 454, 455, and 456, respectively; or
(b) (i) a VH comprising an amino acid sequence having at least 90% sequence identity to the amino acid sequence of SEQ ID NO: 343 or SEQ ID NO: 344; or an amino acid sequence encoded by a nucleotide sequence having at least 80% sequence identity to the sequence of SEQ ID NO: 133;
(ii) a VL comprising an amino acid sequence having at least 90% sequence identity to the amino acid sequence of SEQ ID NO: 345; or an amino acid sequence encoded by a nucleotide sequence having at least 80% sequence identity to the sequence of SEQ ID NOs: 272; or
(iii) a combination thereof.

87. The multispecific molecule of claim 82, wherein the anti-CCR2 binding moiety comprises a VH and a VL comprising the amino acid sequences of: SEQ ID NOs: 343 and 345, respectively, or SEQ ID NOs: 344 and 345, respectively.

88. The multispecific molecule of claim 82, wherein the anti-CCR2 binding moiety does not comprise a VH comprising the amino acid sequence of SEQ ID NO: 480, a VL comprising the amino acid sequence of SEQ ID NO: 481, or a combination thereof.

89. The multispecific molecule of claim 82, comprising an amino acid sequence having at least 80% sequence identity to any one sequence selected from the group consisting of SEQ ID NOs: 373, 136, 131, and 127, or an amino acid sequence encoded by a nucleotide sequence having at least 80% sequence identity to any one sequence selected from the group consisting of SEQ ID NOs: 134, 137, 132, and 128.

90. The multispecific molecule of claim 82, comprising:
(i) the amino acid sequence of SEQ ID NO: 127, the amino acid sequence of SEQ ID NO: 131, and the amino acid sequence of SEQ ID NO: 373; or
(ii) the amino acid sequence of SEQ ID NO: 136, the amino acid sequence of SEQ ID NO: 131, and the amino acid sequence of SEQ ID NO: 373.

91. The multispecific molecule of claim 82, wherein the TGF-beta inhibitor comprises a TGFBR1 polypeptide, a TGFBR2 polypeptide, a TGFBR3 polypeptide, or any combination thereof.

92. The multispecific molecule of claim 91, wherein:
(a) the TGFBR1 polypeptide comprises:
(i) an extracellular domain of TGFBR1 or a functional variant thereof,
(ii) a sequence having at least 80% sequence identity to an extracellular domain of SEQ ID NO: 95, 96, 97, 120, 121, or 122; or
(iii) a sequence having at least 80% sequence identity to the sequence of SEQ ID NO: 104 or SEQ ID NO: 105;
(b) the TGFBR2 polypeptide comprises:
(i) an extracellular domain of TGFBR2 or a functional variant thereof,
(ii) a sequence having at least 80% sequence identity to an extracellular domain of SEQ ID NO: 98, 99, 123, or 124; or
(iii) a sequence having at least 80% sequence identity to any one sequence selected from the group consisting of SEQ ID NOs: 100, 101, 102, and 103;
(c) a TGFBR3 polypeptide, wherein the TGFBR3 polypeptide comprises:
(i) an extracellular domain of TGFBR3 or a functional variant thereof,
(ii) a sequence having at least 80% sequence identity to an extracellular domain of SEQ ID NO: 106, 107, 125, or 126; or
(iii) a sequence having at least 80% sequence identity to the sequence of SEQ ID NO: 108; or
(d) any combination thereof.

93. The multispecific molecule of claim 91,
wherein the TGF-beta inhibitor comprises a first TGF-beta receptor polypeptide and a second TGF-beta receptor polypeptide, and
wherein the first TGF-beta receptor polypeptide and the second TGF-beta receptor polypeptide form:
(a) a homodimer, and the first TGF-beta receptor polypeptide and the second TGF-beta receptor polypeptide are:
(i) two TGFBR1 (TGF beta receptor 1) polypeptides that form a homodimer,
(ii) two TGFBR2 (TGF beta receptor 2) polypeptides that form a homodimer, or
(iii) two TGFBR3 (TGF beta receptor 3) polypeptides that form a homodimer; or
(b) a heterodimer, and the first TGF-beta receptor polypeptide and the second TGF-beta receptor polypeptide are:
(i) a TGFBR1 polypeptide and a TGFBR2 polypeptide that form a heterodimer,
(ii) a TGFBR1 polypeptide and a TGFBR3 polypeptide that form a heterodimer, or
(iii) a TGFBR2 polypeptide and a TGFBR3 polypeptide that form a heterodimer.

94. The multispecific molecule of claim 82,
wherein the TGF-beta inhibitor comprises a first TGF-beta receptor polypeptide and a second TGF-beta receptor polypeptide,
wherein the multispecific molecule comprises a first Fc region and a second Fc region, and wherein:
(i) the first TGF-beta receptor polypeptide is linked to the first Fc region, and
(ii) the second TGF-beta receptor polypeptide is linked to the second Fc region.

95. The multispecific molecule of claim 94, wherein:
(i) the first TGF-beta receptor polypeptide is linked to the C-terminus or the N-terminus of the first Fc region,
(ii) the second TGF-beta receptor polypeptide is linked to the C-terminus or the N-terminus of the second Fc region, or
(iii) a combination thereof.

96. The multispecific molecule of claim 94, wherein:
(i) the first TGF-beta receptor polypeptide is linked to the first Fc region via a linker,
(ii) the second TGF-beta receptor polypeptide is linked to the second Fc region via a linker, or
(iii) a combination thereof.

97. The multispecific molecule of claim 94, wherein the multispecific molecule comprises:
(i) the amino acid sequences of SEQ ID NO: 192 and SEQ ID NO: 193,
(ii) the amino acid sequences of SEQ ID NO: 192 and SEQ ID NO: 195,
(iii) the amino acid sequences of SEQ ID NO: 194 and SEQ ID NO: 193, or
(iv) the amino acid sequences of SEQ ID NO: 194 and SEQ ID NO: 195.

98. The multispecific molecule of claim 82,
wherein the TGF-beta inhibitor comprises a first TGF-beta receptor polypeptide and a second TGF-beta receptor polypeptide,
wherein the multispecific molecule comprises a heavy chain constant region 1 (CH1) and a light chain constant region (CL), and
wherein:
(i) the first TGF-beta receptor polypeptide is linked to the CH1, and
(ii) the second TGF-beta receptor polypeptide is linked to the CL.

99. The multispecific molecule of claim 98, wherein:
(i) the first TGF-beta receptor polypeptide is linked to the N-terminus or the C-terminus of the CH1;
(ii) the second TGF-beta receptor polypeptide is linked the N-terminus or the C-terminus of the CL, or
(iii) a combination thereof.

100. The multispecific molecule of claim 98, wherein:
(i) the first TGF-beta receptor polypeptide is linked to the CH1 via a linker,
(ii) the second TGF-beta receptor polypeptide is linked to the CL via a linker, or
(iii) a combination thereof.

101. The multispecific molecule of claim 98, wherein the multispecific molecule comprises:
(i) the amino acid sequences of SEQ ID NO: 196 and SEQ ID NO: 198,
(ii) the amino acid sequences of SEQ ID NO: 196 and SEQ ID NO: 199,
(iii) the amino acid sequences of SEQ ID NO: 197 and SEQ ID NO: 198, or
(iv) the amino acid sequences of SEQ ID NO: 197 and SEQ ID NO: 199.

102. The multispecific molecule of claim 82, wherein:
(i) the TGF inhibitor is linked to the anti-CSF1R binding moiety or the anti-CCR2 binding moiety;
(ii) the anti-CSF1R binding moiety, the anti-CCR2 binding moiety, or a combination thereof comprises an Fc region, wherein the TGF inhibitor is linked to the Fc region;
(iii) the multispecific molecule comprises a first TGF-beta inhibitor and a second TGF-beta inhibitor, wherein the first TGF-beta inhibitor is linked to the anti-CSF1R binding moiety, and the second TGF-beta inhibitor is linked to the anti-CCR2 binding moiety;
(iv) the multispecific molecule comprises a first TGF-beta inhibitor and a second TGF-beta inhibitor,
wherein the anti-CSF1R binding moiety comprises a first Fc region, and the anti-CCR2 binding moiety comprises a second Fc region, and
wherein the first TGF-beta inhibitor is linked to the first Fc region, and the second TGF-beta inhibitor is linked to the second Fc region; or
(v) the multispecific molecule comprises a first TGF-beta inhibitor and a second TGF-beta inhibitor,
wherein the anti-CSF1R binding moiety comprises a first light chain and a first heavy chain,
wherein the first heavy chain comprises a first Fc region, and
wherein the first TGF-beta inhibitor is linked to the first Fc region; and
wherein the anti-CCR2 binding moiety comprises a second light chain and a second heavy chain,
wherein the second heavy chain comprises a second Fc region, and
wherein the second TGF-beta inhibitor is linked to the second Fc region.

103. The multispecific molecule of claim 102, wherein:
(i) the TGF inhibitor is linked to the C-terminus or the N-terminus of the anti-CSF1R binding moiety or the C-terminus or the N-terminus of the anti-CCR2 binding moiety;
(ii) the TGF inhibitor is linked to the C-terminus or the N-terminus of the Fc region of the anti-CSF1R binding moiety or the C-terminus or the N-terminus of the Fc region of the anti-CCR2 binding moiety;
(iii) the first TGF-beta inhibitor is linked to the C-terminus or the N-terminus of the anti-CSF1R binding moiety, the C-terminus or the N-terminus of the second TGF-beta inhibitor is linked to the anti-CCR2 binding moiety, or a combination thereof;
(iv) the first TGF-beta inhibitor is linked to the C-terminus or the N-terminus of the first Fc region of the anti-CSF1R binding moiety, the second TGF-beta inhibitor is linked to the C-terminus or the N-terminus of the second Fc region of the anti-CCR2 binding moiety, or a combination thereof; or
(v) the first TGF-beta inhibitor is linked to the C-terminus or the N-terminus of the first Fc region of the first heavy chain, the second TGF-beta inhibitor is linked to the C-terminus or the N-terminus of the second Fc region of the second heavy chain, or a combination thereof.

104. The multispecific molecule of claim 102, wherein:
(i) the TGF inhibitor is linked to the anti-CSF1R binding moiety or the anti-CCR2 binding moiety via a linker;
(ii) the TGF inhibitor is linked to the Fc region of the anti-CSF1R binding moiety or the Fc region of the anti-CCR2 binding moiety via a linker,
(iii) the first TGF-beta inhibitor is linked to the anti-CSF1R binding moiety via a linker, the second TGF-beta inhibitor is linked to the anti-CCR2 binding moiety via a linker, or a combination thereof;
(iv) the first TGF-beta inhibitor is linked to the first Fc region of the anti-CSF1R binding moiety via a linker, the second TGF-beta inhibitor is linked to the second Fc region of the anti-CCR2 binding moiety via a linker, or a combination thereof; or
(v) the first TGF-beta inhibitor is linked to the first Fc region of the first heavy chain via a linker, the second TGF-beta inhibitor is linked to the second Fc region of the second heavy chain via a linker, or a combination thereof.

105. A recombinant polynucleotide comprising a sequence encoding the multispecific molecule of claim 82.

106. A pharmaceutical composition comprising the multispecific molecule of claim 82, and a pharmaceutically acceptable carrier, excipient, or stabilizer.

107. A method of treating a cancer in a subject in need thereof, comprising administering to the subject the pharmaceutical composition of claim 106, wherein the pharmaceutical composition is administered in an amount effective to treat the cancer.

108. An antibody molecule that binds to CSF1R, comprising:
(a) a heavy chain variable region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3,
wherein the HCDR1, the HCDR2, the HCDR3, the LCDR1, the LCDR2, and the LCDR3 comprise:
(i) the amino acid sequences of SEQ ID NOs: 402, 474, 475, 432, 434, and 436, respectively;
(ii) the amino acid sequences of SEQ ID NOs: 402, 474, 475, 433, 435, and 437, respectively;
(iii) the amino acid sequences of SEQ ID NOs: 403, 478, 479, 433, 435, and 437, respectively;
(iv) the amino acid sequences of SEQ ID NOs: 403, 478, 479, 432, 434, and 436, respectively;
(v) the amino acid sequences of SEQ ID NOs: 402, 405, 413, 432, 434, and 436, respectively; or
(vi) the amino acid sequences of SEQ ID NOs: 403, 411, 422, 433, 435, and 437, respectively; or
(b) (i) a VH comprising an amino acid sequence having at least 80% sequence identity to the sequence of SEQ ID NO: 324, and a VL comprising an amino acid sequence having at least 80% sequence identity to the sequence of SEQ ID NO: 341; or
(ii) a VH comprising an amino acid sequence having at least 80% sequence identity to the sequence of SEQ ID NO: 339, and a VL comprising an amino acid sequence having at least 80% sequence identity to the sequence of SEQ ID NOs: 139.

109. An antibody molecule that binds to CCR2, comprising:
(a) a heavy chain variable region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3,
wherein the HCDR1, the HCDR2, the HCDR3, the LCDR1, the LCDR2, and the LCDR3 comprise the amino acid sequences of SEQ ID NOs: 446, 447, 448, 454, 455, and 456, respectively, and
wherein:
(i) the VH does not comprise the amino acid sequence of SEQ ID NO: 480,
(ii) the VL does not comprise the amino acid sequence of SEQ ID NO: 481, or
(iii) a combination thereof; or
(b) a VH comprising an amino acid sequence having at least 90% sequence identity to the sequence of SEQ ID NO: 343, and a VL comprising an amino acid sequence having at least 90% sequence identity to the sequence of SEQ ID NO: 345.
